(19) United States
(12) Patent Application Publication
(10) Pub. No.: US 2023/0416262 A1
(43) Pub. Date: Dec. 28, 2023

(54) TRPML MODULATORS

(71) Applicant: Casma Therapeutics, Inc., Cambridge, MA (US)

(72) Inventors: Andrew Thomas Maynard, Winchester, MA (US); Erik Lee Meredith, Hudson, MA (US); Jeffrey Owen Saunders, Lincoln, MA (US); Jian Lin, Acton, MA (US); Jorge Garcia Fortanet, Wilmington, MA (US)

(21) Appl. No.: 18/019,638

(22) PCT Filed: Aug. 6, 2021
(86) PCT No.: PCT/US21/44911

§ 371 Date: Feb. 3, 2023

Related U.S. Application Data
(60) Provisional application No. 63/062,943, filed on Aug. 7, 2020.
(60) Provisional application No. 63/119,915, filed on Dec. 1, 2020.
(60) Provisional application No. 63/186,846, filed on May 11, 2021.

(51) Int. Cl. C07D 487/10; C07D 209/96; C07D 265/18; C07D 405/12; C07D 403/12; C07D 405/14; C07D 409/14; C07D 471/10; C07D 491/107

(52) U.S. Cl. CPC C07D 487/10; C07D 209/96; C07D 265/18; C07D 405/12; C07D 491/107; C07D 405/14; C07D 409/14; C07D 471/10; C07D 403/12

(57) ABSTRACT

The present invention provides compounds, pharmaceutically acceptable compositions thereof, and methods of using the same.

### CROSS-REFERENCE TO RELATED APPLICATIONS

The present application claims priority to U.S. Provisional Application No. 63/062,943, filed Aug. 7, 2020; U.S. Provisional Application No. 63/119,915, filed Dec. 1, 2020; and U.S. Provisional Application No. 63/186,846, filed May 11, 2021, each of which is incorporated by reference in its entirety.

### BACKGROUND

Transient Receptor Potential Mucolipin-1 (also known as TRPML1 or ML1) is a Ca²⁺ channel in the lysosome that regulates certain aspects of lysosome trafficking, including autophagy. See Wang, el al., PNAS, E1373-E1381 (Mar. 2, 2015). In particular, TRPML1 is an inwardly rectifying current channel that transports cations from the lumen of the lysosome to the cytosol. See Di Paolda, et al., Cell Calcium 69:112-121 (2018). Release of Ca²⁺ from the lysosome via TRPML1 modulates transcription factor EB activity. See Medina, et al., Nat. Cell. Biol., 17(3):288-299 (2015).

### SUMMARY

It has recently been discovered that upregulation of autophagy is beneficial to patients suffering from a number of diseases and disorders. For example, it has been reported that inducing autophagy promotes clearance of hepatotoxic alpha-1-anti-trypsin (ATZ) in the liver. See Pastore, et al., EMBO Mol. Med. 5(3): 397-412 (March 2013). Moreover, autophagy was recently found to be useful in the treatment of neurodegenerative disorders, cancer, and heart disease. See Pierzynowska, et al., Metab. Brain Dis., 33(4); 989-1008 (2018) (discussing neurodegenerative disorders); Nelson & Shacka, Curr. Pathobiol. Rep., 1(4): 239-245 (2013) (discussing cancer); Sciaretta, et al., Annual Review of Physiology, 80:1-26 (2018) (discussing heart disease); Maiuri & Kroemer, Cell Death & Differentiation, 26: 680-689 (2019) (discussing therapeutic applications of autophagy, generally).

The present disclosure provides, among other things, technologies for regulating (e.g., up-regulating) autophagy. For example, in some embodiments, the present disclosure demonstrates effectiveness of certain approaches to TRPML modulation (e.g., TRPML agonism, including agonism of TRPML1, TPRML2, and/or TRPML3) in enhancing autophagy. Thus, among other things, the present disclosure demonstrates that targeting a TRPML (e.g., TRPML1, TRPML2, and/or TRPML3) as described herein can enhance autophagy.

The present disclosure also provides certain technologies for use in medicine, and in particular for treating certain diseases, disorders or conditions and/or for identifying, characterizing, and/or manufacturing certain agents and/or compositions or that comprise or deliver them that are useful in treating such diseases, disorders or conditions.

In some embodiments, the present disclosure demonstrates that modulating (e.g., agonizing) a TRPML (e.g., TRPML1, TRPML2, and/or TRPML3) and/or otherwise enhancing autophagy is useful in the treatment of certain diseases, disorders or conditions.

It is, therefore, desirable to identify methods and modes of promoting autophagy. Given TRPML's role in autophagy, described herein are TRPML (e.g., TRPML1, TRPML2, and/or TRPML3) modulators useful for promoting autophagy and/or treating certain diseases, disorders, or conditions.

In particular, the present application provides technologies useful for modulating a TRPML (e.g., TRPML1, TRPML2, and/or TRPML3).

In some embodiments, the present disclosure provides and/or utilizes TRMPL modulators that are small molecule compounds having a chemical structure as indicated below in Formula I:

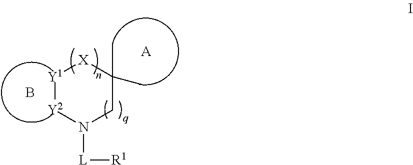

or a pharmaceutically acceptable salt thereof,
wherein
X is —NR⁵—, —C(R⁵)₂, —C(O)—, — or —O—;
each of Y¹ and Y² is independently selected from N and C;
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
B is a fused optionally substituted C₅-C₆ aryl or optionally substituted 5- to 6-membered heteroaryl comprising 1 to 3 heteroatoms selected from N, O, and S;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S; each R^{2a} and each R^{2b} are independently selected from H and an optionally substituted group selected from C₁-C₆ aliphatic, C₃-C₁₂cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
each R³ is independently halo, —S(O)₂—NR^{3a}R^{3b}, —S(O)₂—R^{3b}, —S(NR^{3c})—(O)—NR^{3a}R^{3b}, —S(O)(NR^{3c})—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, OH, and optionally substituted C₁-C₆ aliphatic;
each R⁵ is independently selected from hydrogen, halo, —CN, and optionally substituted C₁-C₆ aliphatic;
R⁸ is selected from H and optionally substituted C₁-C₆ aliphatic;
n is 0 or 1;
m is 0 to 4;
p is 0 to 4; and
q is 1 or 2.

### Definitions

Agonist. As used herein, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). In some embodiments, “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule. Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The terms “alkylene” and “alkylenyl” are used interchangeably and refer to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” or “-enyl” when appended to certain groups herein are intended to refer to a bifunctional moiety of said group. For example, “-ene” or “-enyl”, when appended to “cyclopropyl” becomes “cyclopropylene” or “cyclopropylenyl” and is intended to refer to a bifunctional cyclopropyl group, e.g.,

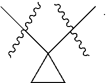 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkenylene: The term “alkenylene” and “alkenylenyl” are used interchangeably and refers to a bivalent alkenyl group. In some embodiments, “alkenylene” is a bivalent straight or branched alkenyl group.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Alkynylene: The term “alkynylene” and “alkynylenyl” are used interchangeably and refers to a bivalent alkynyl group. In some embodiments, “alkynylene” is a bivalent straight or branched alkynyl group.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Aryl: The term “aryl” refers to monocyclic and bicyclic ring systems having a total of five to fourteen ring members (e.g., C₅-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

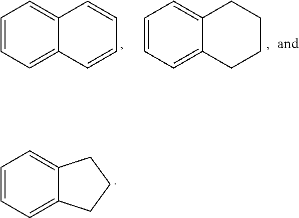

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens, feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to a to an entity (or form thereof) whose presence, or level, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, in some embodiments, a small molecule may be considered to be engineered if its structure and/or production is designed and/or implemented by the hand to man. Analogously, in some embodiments, a polynucleotide may be considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, expression products of an engineered polynucleotide, and/or progency of an engineered polynucleotide or cell are typically still referred to as “engineered” even though the actual manipulation was performed on a prior entity.

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 12 ring atoms (e.g., 5- to 6-membered monocyclic heteroaryl or 9- to 12-membered bicyclic heteroaryl); having 6, 10, or 14 n-electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 7- to 12-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, tetrahydroquinolinyl,

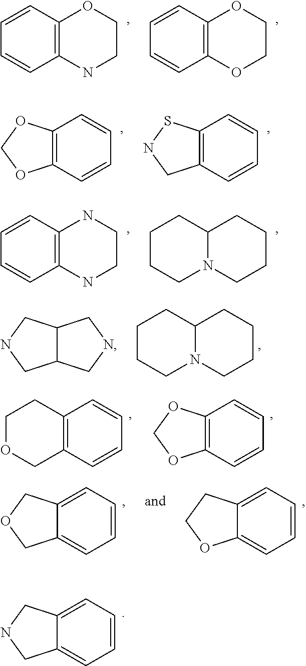

A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms. Exemplary bridged ring systems include

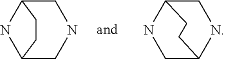

Exemplary polycyclic heterocyclic ring systems that are spirocyclic include

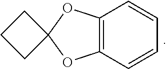 [SMILES: c1ccc2c(c1)OC1(CCC1)O2]

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polycyclic: As used herein, the term “polycyclic” refers to a saturated or unsaturated ring system having two or more rings (for example, heterocyclyl rings, heteroaryl rings, cycloalkyl rings, or aryl rings), having between 7 and 20 atoms, in which one or more carbon atoms are common to two adjacent rings. For example, in some embodiments, a polycyclic ring system refers to a saturated or unsaturated ring system having three or more rings (for example, heterocyclyl rings, heteroaryl rings, cycloalkyl rings, or aryl rings), having between 14 and 20 atoms, in which one or more carbon atoms are common to two adjacent rings. The rings in a polycyclic ring system may be fused (i.e., bicyclic or tricyclic), spirocyclic, or a combination thereof.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest. In some embodiments, a source of interest is a biological or environmental source. In some embodiments, a source of interest may be or comprise a cell, tissue, or organism, such as a microbe, a plant, or an animal (e.g., a human). In some embodiments, a source of interest is or comprises biological tissue or fluid. In some embodiments, a source of interest may be or comprise a preparation generated in a production run. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample.

Specific: The term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, a binding agent binds with higher on-rate, lower off-rate, increased affinity, decreased dissociation, and/or increased stability to its target entity as compared with the competing alternative target(s).

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

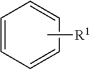

refers to at least

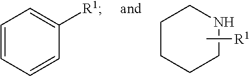

refers to at least

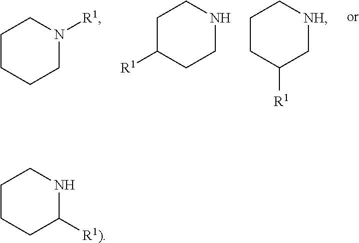

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)-4SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; C(S)R^{∘}; —CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁ Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂, —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{•} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁-Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ⁵N for ¹⁴N; ¹⁷O or ¹⁸O for 16O; ³⁶Cl for ^{35/37}C; ¹⁸F for ¹⁹F; ¹³¹I for ¹²⁷I; etc). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will appreciate that a bond designated as [symbol] in a small molecule structure, as used herein, refers to a bond that, in some embodiments, is a single (e.g., saturated) bond, and in some embodiments, is a double (e.g., unsaturated) bond. For example, the following structure:

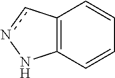

is intended to encompass both

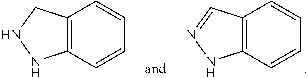

Those skilled in the art will further appreciate that, in small molecule structures, the symbol [symbol] , as used herein, refers to a point of attachment between two atoms. Additionally or alternatively, the symbol [symbol] refers to a point of attachment ring in a spirocyclic manner.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### DETAILED DESCRIPTION

### TRPML and Autophagy

Autophagy is a mechanism of the cell that degrades cytoplasmic material and organelles. There are multiple types of autophagy: (1) macroautophagy (generally referred to as autophagy); (2) microautophagy, and (3) chaperone-mediated autophagy. See Eskelinen & Saftig, Biochimica et Biophysica Acta—Mol. Cell Res., 1793(4):664-673 (2009). In macroautophagy, the autophagosome engulfs waste materials in the cytoplasm and fuses to the lysosome, where materials are delivered for degradation. The lysosome is as a subcellular organelle containing more than 50 soluble acid hydrolases useful for digesting cellular components. Fusion of the lysosome to the autophagosome is activated, in part, by release of ions through ion channels in the membrane of the lysome, including Ca²⁺. See Cao, et al., J. Bio. Chem., 292(20)8424-8435 (2017).

Transient Receptor Potential Mucolipin-1 (also known as TRPML1 or ML1) is a Ca²⁺ channel in the lysosome that regulates autophagy. See Wang, et al., PNAS, E1373-E1381 (Mar. 2, 2015). In particular, TRPML1 is an inwardly rectifying current channel that transports cations from the lumen of the lysosome to the cytosol. See Di Paolda, et al., Cell Calcium 69:112-121 (2018). Release of Ca²⁺ from the lysosome via TRPML1 modulates transcription factor EB activity via local calcineurin activation, which ultimately induces autophagy and lysosomal biogenesis. See Medina, et al., Nat. Cell. Biol., 17(3):288-299 (2015).

It has recently been discovered that upregulation of autophagy is beneficial to patients suffering from a number of diseases and disorders. For example, it has been reported that inducing autophagy promotes clearance of hepatotoxic alpha-1-anti-trypsin (ATZ) in the liver. See Pastore, et al., EMBO Mol. Med. 5(3): 397-412 (March 2013). Moreover, autophagy was recently found to be useful in the treatment of neurodegenerative disorders, cancer, and heart disease. See Pierzynowska, el al., Metab. Brain Dis., 33(4); 989-1008 (2018) (discussing neurodegenerative disorders); Nelson & Shacka, Curr. Pathobiol. Rep., 1(4): 239-245 (2013) (discussing cancer); Sciaretta, et al., Annual Review of Physiology, 80:1-26 (2018) (discussing heart disease); Maiuri & Kroemer, Cell Death & Differentiation, 26: 680-689 (2019)(discussing therapeutic applications of autophagy, generally). It is, therefore, desirable to identify methods and modes of promoting autophagy. Given TRPML's role in autophagy, described herein are TRPML1 modulators useful for promoting autophagy and/or treating certain diseases, disorders, or conditions.

The present disclosure provides the insight that TRMPL may represent a particularly desirable target that, among other things, may permit modulation (e.g., enhancement) of autophagy in certain contexts.

### TRPML Modulators

### Structure

In some embodiments, the present disclosure provides and/or utilizes TRMPL modulators (e.g., TRPML1, TPRML2, and/or TPRML3) that are small molecule compounds having a chemical structure as indicated below in Formula I:

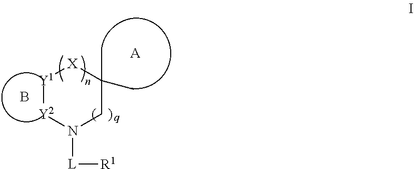

or a pharmaceutically acceptable salt thereof,
wherein
X is —NR⁵—, —C(R⁵)₂—, —C(O)—, or —O—;
each of Y¹ and Y² is independently selected from N and C;
L is an optionally substituted group selected from —C₀-C₆ alkylenyl-S(O)₂—, —S(O)₂—C₁-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
B is a fused optionally substituted C₅-C₆ aryl or optionally substituted 5- to 6-membered heteroaryl comprising 1 to 3 heteroatoms selected from N, O, and S;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR²R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
each R³ is independently halo, —S(O)₂—NR^{3a}R^{3b}, —S(O)₂—R^{3b}, —S(O)(NR)—NR^{3a}R^{3b}, —S(O)(NR^{3c}C)—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently H, OH, and optionally substituted C₁-C₆ aliphatic;
each R⁵ is independently selected from hydrogen, halo, —CN, and optionally substituted C₁-C₆ aliphatic; Rx is selected from H and optionally substituted C₁-C₆ aliphatic;
n is 0 or 1;
m is 0 to 4;
p is 0 to 4; and
q is 1 or 2.

As defined generally above, and in any formula described herein, X is —NR³—, —C(R⁵)₂—, —C(O)—, or —O—. In some embodiments, X is —NR⁵—. In some embodiments, X is —NH—. In some embodiments, X is —C(R⁵)₂—. In some embodiments, X is —CH(R⁵)—. In some embodiments, X is —CH₂—. In some embodiments, X is —CH(CH₃)—. In some embodiments, X is —O—. In some embodiments, X is —C(O)—.

As defined generally above, Y¹ and Y² are each independently N or C. In some embodiments, Y¹ and Y² are each N. In some embodiments, Y¹ and Y² are each C. In some embodiments, Y¹ is N and Y² is C. In some embodiments, Y¹ is C and Y² is N.

As defined generally above, L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl.

In some embodiments, L is optionally substituted —C₀-C₆ alkylenyl-S(O)₂—. In some embodiments, L is —S(O)₂—. In some embodiments, L is optionally substituted —C₁-C₆ alkylenyl-S(O)₂—. In some embodiments, L is-C₁-C₆ alkylenyl-S(O)₂— substituted with halogen, —(CH₂)₀₋₄R^{∘}, —(CH₂)₀₋₄OR^{∘}, or —O(CH₂)₀₋₄R^{∘}. In some embodiments, L is optionally substituted —C₁-C₃ alkylenyl-S(O)₂—. In some embodiments, L is-C₁-C₃ alkylenyl-S(O)₂— substituted with halogen, —(CH₂)₀₋₄R^{∘}, —(CH₂)₀₋₄OR^{∘}, or —O(CH₂)₀₋₄R^{∘}. In some embodiments, L is —CH₂—S(O)₂—. In some embodiments, L is —CH(CH₃)—S(O)₂—. In some embodiments, L is —CH(R^{∘})—S(O)₂—.

In some embodiments, L is optionally substituted —S(O)₂—C₁-C₆ alkylenyl. In some embodiments, L is-S(O)₂—C₁-C₆ alkylenyl substituted with halogen, —(CH₂)₀₋₄R^{∘}, —(CH₂)₀₋₄OR^{∘}, or —O(CH₂)₀₋₄R^{∘}. In some embodiments, L is optionally substituted —S(O)₂—C₁-C₃ alkylenyl. In some embodiments, L is-S(O)₂—C₁-C₃ alkylenyl substituted with halogen, —(CH₂)₀₋₄R^{∘}, —(CH₂)₀₋₄OR^{∘}, or —O(CH₂)₀₋₄R^{∘}. In some embodiments, L is —S(O)₂—CH₂—. In some embodiments, L is —S(O)₂—CH(CH₃)—. In some embodiments L is —S(O)₂—CH(R^{∘})—. In some embodiments, L is

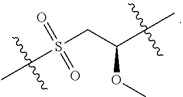 [SMILES: *[C@H](CS(*)(=O)=O)OC]

In some embodiments, L is

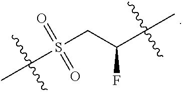 [SMILES: *[C@@H](F)CS(*)(=O)=O]

In some embodiments, L is optionally substituted —S(O)—C₁-C₆ alkylenyl. In some embodiments, L is-S(O)—C₁-C₆ alkylenyl substituted with halogen, —(CH₂)₀₋₄R^{∘}, —(CH₂)₀₋₄OR^{∘}, or —O(CH₂)₀₋₄R^{∘}. In some embodiments, L is optionally substituted —S(O)—C₁-C₃ alkylenyl. In some embodiments, L is —S(O)—C₁-C₃ alkylenyl substituted with halogen, —(CH₂)₀₋₄R^{∘}, —(CH₂)₀₋₄ OR^{∘}, or —O(CH₂)₀₋₄R^{∘}. In some embodiments, L is —S(O)—CH₂—. In some embodiments, L is —S(O)—CH(CH₃)—.

In some embodiments, L is optionally substituted —C₀-C₆ alkylenyl-S(O)—. In some embodiments, L is —S(O)—. In some embodiments, L is optionally substituted —C₁-C₆ alkylenyl-S(O)—. In some embodiments, L is-C₁-C₆ alkylenyl-S(O)— substituted with halogen, —(CH₂)₀₋₄R^{∘}, —(CH₂)₀₋₄OR^{∘}, or —O(CH₂)₀₋₄R^{∘}. In some embodiments, L is optionally substituted —C₁-C₃ alkylenyl-S(O)—. In some embodiments, L is-C₁-C₃ alkylenyl-S(O)— substituted with halogen, —(CH₂)₀₋₄R^{∘}, —(CH₂)₀₋₄OR^{∘}, or —O(CH₂))₄R^{∘}. In some embodiments, L is —CH₂—S(O)—. In some embodiments, L is —CH(CH₃)—S(O)—. In some embodiments, L is —CH(R^{∘})—S(O)—.

In some embodiments, L is optionally substituted —C(O)—C₀-C₆ alkylenyl. In some embodiments, L is —C(O)—. In some embodiments, L is-C(O)—C₁-C₆ alkylenyl substituted with halogen, —(CH₂)₀₋₄R^{∘}, —(CH₂)₀₋₄OR^{∘}, or —O(CH₂)—R^{∘}. In some embodiments, L is optionally substituted —C(O)—C₁-C₃ alkylenyl. In some embodiments, L is-C(O)—C₁-C₃ alkylenyl substituted with halogen, —(CH₂)₀₋₄R^{∘}, —(CH₂)₀₋₄OR^{∘}, or —O(CH₂)₀₋₄R^{∘}. In some embodiments, L is —C(O)—CH₂—. In some embodiments, L is —C(O)—CH(CH₃)—.

In some embodiments, L is optionally substituted —C(O)—O—C₀-C₆ alkylenyl. In some embodiments, L is —C(O)—O—.

In some embodiments, L is optionally substituted —C(O)—N(R⁸)—C₀-C₆ alkylenyl. In some embodiments, L is optionally substituted —C(O)—N(H)—C₀-C₆ alkylenyl. In some embodiments, L is optionally substituted —C(O)—N(C₁-C₆ aliphatic)-C₀-C₆ alkylenyl. In some embodiments, L is optionally substituted —C(O)—N(R⁸)—. In some embodiments, L is —C(O)—NH—. In some embodiments, L is —C(O)—N(CH₃)—.

In some embodiments, L is optionally substituted —C₁-C₆ alkylenyl. In some embodiments, L is optionally substituted —C₁-C₃ alkylenyl. In some embodiments, L is —C₁-C₆ alkylenyl. In some embodiments, L is —C₁-C₆ alkylenyl substituted with halogen, —(CH₂)₀₋₄R^{∘}, —(CH₂)₀₋₄OR^{∘}, or —O(CH₂)₀₋₄R^{∘}. In some embodiments, L is —C₁-C₃ alkylenyl substituted with halogen, —(CH₂)₀₋₄R^{∘}, —(CH₂)₀₋₄OR^{∘}, or —O(CH₂)₀₋₄R^{∘}. In some embodiments, L is —CH₂—. In some embodiments, L is —CH₂—CH₂—. In some embodiments, L is —CH(CH₃)—.

In some embodiments, L is optionally substituted C₃-C₆ cycloalkylenyl. In some embodiments, L is optionally substituted C₃ cycloalkylenyl. In some embodiments, L is optionally substituted cyclopropylenyl. In some embodiments, L is optionally substituted C₄ cycloalkylenyl. In some embodiments, L is optionally substituted cyclobutylenyl. In some embodiments, L is optionally substituted C₅ cycloalkylenyl. In some embodiments, L is optionally substituted cyclopentylenyl. In some embodiments, L is optionally substituted C₆ cycloalkylenyl. In some embodiments, L is optionally substituted cyclohexylenyl. In some embodiments, L is

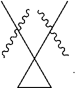 [SMILES: *C1(*)CC1]

In some embodiments, L is selected from —S(O)₂—, —S(O)₂—CH₂—, —S(O)₂—CH(CH₃)—, —CH₂—S(O)₂—,

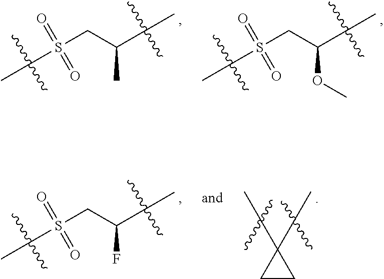

As defined generally above, A is C₃-C₁₂ cycloaliphatic or 3- to 12-membered heterocyclyl comprising 1 to 3 heteroatoms selected from N, O, and S, wherein A is substituted with (R²)ₘ. In some embodiments, A is C₃-C₁₂ cycloaliphatic. In some embodiments, A is C₃-C₁₀ cycloaliphatic. In some embodiments, A is cyclopropyl, cyclobutyl, cyclopentyl, cyclohexyl, or cycloheptyl. In some embodiments, A is cyclopentyl or cyclohexyl. In some embodiments, A is:

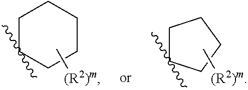

In some embodiments, A is

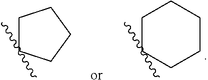

In some embodiments, A is 3- to 12-membered heterocyclyl comprising 1 to 3 heteroatoms selected from N, O, and S. In some embodiments, A is 4- to 6-membered heterocyclyl comprising 1 to 3 heteroatoms selected from N, O, and S. In some embodiments, A is 4-membered heterocyclyl comprising 1 to 3 heteroatoms selected from N, O, and S. In some embodiments, A azetidinyl. In some embodiments, A is 5-membered heterocyclyl comprising 1 to 3 heteroatoms selected from N, O, and S. In some embodiments, A is pyrrolidinyl. In some embodiments, A is 6-membered heterocyclyl comprising 1 to 3 heteroatoms selected from N, O, and S. In some embodiments, A is piperidinyl or tetrahydropyranyl. In some embodiments, A is pyrrolidinyl, piperidinyl, tetrahydropyranyl, or tetrahyrothiopyranyl. In some embodiments, A is pyrrolidinyl or piperidinyl. In some embodiments, A is

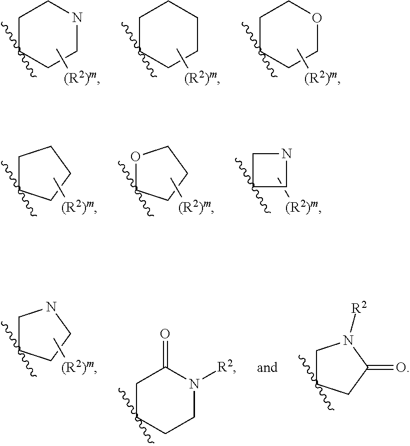

In some embodiments, A is

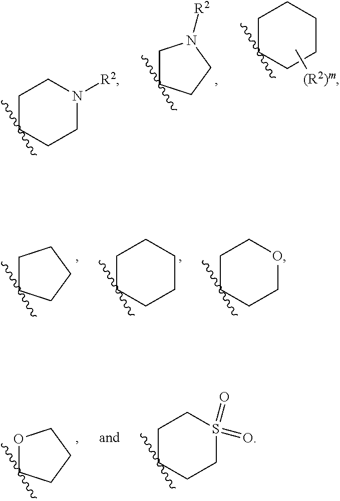

In some embodiments, A is substituted with (R²)ₘ. In some embodiments, A is substituted with 0, 1, 2, 3, or 4 instances of R². In some embodiments, A is substituted with 0 instances of R² (i.e., A is unsubstituted). In some embodiments, A is substituted with 1 R². In some embodiments, A is substituted with 2 R². In some embodiments, A is substituted with 3 R². In some embodiments, A is substituted with 4 R².

As defined generally above, B is a fused optionally substituted C₅-C₆ aryl or optionally substituted 5- to 6-membered heteroaryl comprising 1 to 3 heteroatoms selected from N, O, and S. In some embodiments, B is a fused C₅-C₆ aryl or 5- to 6-membered heteroaryl comprising 1 to 3 heteroatoms selected from N, O, and S, substituted with one or more instances of halogen, CN, —(CH₂)₀₋₄R^{∘}, —(CH₂)₀₋₄OR^{∘}, or —O(CH₂)₀₋₄R^{∘}. In some embodiments, B is a fused optionally substituted C₅-C₆ aryl. In some embodiments, B is a fused C₅-C₆ aryl. In some embodiments, B is a fused C₅-C₆ aryl substituted with one or more instances of halogen, CN, —(CH₂)₀₋₄R^{∘}, —(CH₂)₀₋₄OR^{∘}, or —O(CH₂)₀₋₄R^{∘}. In some embodiments, B is a fused C₅-C₆ aryl substituted with one or more instances of halogen, CN, or OCH₃. In some embodiments, B is a fused phenyl. In some embodiments, B is a fused phenyl substituted with one or more instances of halogen, CN, or OCH₃.

In some embodiments, B is fused optionally substituted 5- to 6-membered heteroaryl comprising 1 to 3 heteroatoms selected from N, O, and S. In some embodiments, B is fused 5- to 6-membered heteroaryl comprising 1 to 3 heteroatoms selected from N, O, and S. In some embodiments, B is fused 5- to 6-membered heteroaryl comprising 1 to 3 heteroatoms selected from N, O, and S substituted with one or more instances of halogen, CN, —(CH₂)₀₋₄R^{∘}, —(CH₂)₀₋₄OR^{∘}, or —O(CH₂)₀₋₄R^{∘}. In some embodiments, B is fused 5- to 6-membered heteroaryl comprising 1 to 3 heteroatoms selected from N, O, and S substituted with one or more instances of halogen, CN, or OCH₃. In some embodiments, B is fused thienyl. In some embodiments, B is a fused thienyl substituted with one or more instances of halogen, CN, or OCH₃. In some embodiments, B is fused pyridyl. In some embodiments, B is fused pyridyl substituted with one or more instances of, —(CH₂)₀₋₄R^{∘}. In some embodiments, B is fused pyridyl substituted with C₁-C₆aliphatic. In some embodiments, B is fused pyridyl substituted with methyl. In some embodiments, B is fused pyrazolyl. In some embodiments, B is fused pyrazolyl substituted with one or more instances of, —(CH₂)₀₋₄R^{∘}. In some embodiments, B is fused pyrazolyl substituted with C₁-C₆aliphatic. In some embodiments, B is fused pyrazolyl substituted with methyl.

As defined generally above, R¹ is selected from C₁-C₆ aliphatic, C₃-C₁₂cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ.

In some embodiments, R¹ is C₅-C₁₂ aryl substituted with (R³)ₚ, 5- to 12-membered heteroaryl comprising 1 to 3 heteroatoms selected from N, O, and S substituted with (R³)ₚ, or 3- to 12-membered heterocyclyl comprising 1 to 3 heteroatoms selected from N, O, and S substituted with (R³)ₚ.

In some embodiments, R¹ is C₁-C₆ aliphatic substituted with (R³)ₚ. In some embodiments, R¹ is C₁-C₆ aliphatic, wherein p is 0. In some embodiments, R¹ is C₁-C₆ alkyl substituted with (R³)ₚ. In some embodiments, R¹ is C₁-C₃ alkyl substituted with (R³)ₚ. In some embodiments, R¹ is methyl, ethyl, propyl (e.g., n-propyl and iso-propyl), butyl (e.g., n-butyl, iso-butyl, sec-butyl, and tert-butyl), pentyl, or hexyl, optionally substituted with (R³)ₚ.

In some embodiments, R¹ is C₃-C₁₂ cycloaliphatic substituted with (R³)ₚ. In some embodiments, R¹ is C₃-C₁₂ cycloalkyl substituted with (R³)P. In some embodiments, R¹ is C₃-C₆ cycloalkyl substituted with (R³)ₚ. In some embodiments, R¹ is cyclopropyl, cyclobutyl, cyclopentyl, or cyclohexyl substituted with (R³)ₚ.

In some embodiments, R¹ is C₅-C₁₂ aryl substituted with (R³)ₚ. In some embodiments, R¹ is C₃-C₆ aryl substituted with (R³)ₚ. In some embodiments, R¹ is phenyl substituted with (R³)ₚ.

In some embodiments, R¹ is monocyclic or bicyclic 3- to 12-membered heterocyclyl comprising 1 to 3 heteroatoms selected from N, O, and S substituted with (R³)ₚ. In some embodiments, R¹ is monocyclic 3- to 7-membered heterocyclyl comprising 1 to 3 heteroatoms selected from N, O, and S substituted with (R³)ₚ. In some embodiments, R¹ is 4- to 6-membered heterocyclyl comprising 1 to 3 heteroatoms selected from N, O, and S substituted with (R³)ₚ. In some embodiments, R¹ is 4-membered heterocyclyl comprising 1 to 3 heteroatoms selected from N, O, and S substituted with (R³)ₚ. In some embodiments, R¹ is 5-membered heterocyclyl comprising 1 to 3 heteroatoms selected from N, O, and S substituted with (R³)ₚ. In some embodiments, R¹ is 6-membered heterocyclyl comprising 1 to 3 heteroatoms selected from N, O, and S substituted with (R³)ₚ.

In some embodiments, R¹ is bicyclic 10- to 12-membered heterocyclyl comprising 1 to 3 heteroatoms selected from N, O, and S substituted with (R³)ₚ. In some embodiments, R¹ is bicyclic 10- to 12-membered heterocyclyl comprising 1 to 3 heteroatoms selected from N, O, and S, substituted with (R³)ₚ, where one ring of said bicyclic heterocyclyl is a fused aryl ring. In some embodiments, R¹ is bicyclic 10-membered heterocyclyl comprising 1 to 3 heteroatoms selected from N, O, and S substituted with (R³)ₚ. In some embodiments, R¹ is bicyclic 11-membered heterocyclyl comprising 1 to 3 heteroatoms selected from N, O, and S substituted with (R³)P. In some embodiments, R¹ is bicyclic 12-membered heterocyclyl comprising 1 to 3 heteroatoms selected from N, O, and S substituted with (R³)ₚ.

In some embodiments, R¹ is 5- to 12-membered heteroaryl comprising 1 to 3 heteroatoms selected from N, O, and S substituted with (R³)ₚ. In some embodiments, R¹ is 5- to 6-membered heteroaryl comprising 1 to 3 heteroatoms selected from N, O, and S substituted with (R³)ₚ. In some embodiments, R¹ is 5-membered heteroaryl comprising 1 to 3 heteroatoms selected from N, O, and S substituted with (R³)ₚ. In some embodiments, R¹ is 6-membered heteroaryl comprising 1 to 3 heteroatoms selected from N, O, and S substituted with (R³)ₚ. In some embodiments, R¹ is pyrrolyl, imidazolyl, pyrrazolyl, thiophenyl, pyridinyl, or pyrazinyl substituted with (R³)ₚ. In some embodiments, R¹ is pyrrazolyl or thiophenyl substituted with (R³)ₚ.

As defined generally above, R¹ is substituted with p-instances of R³ (i.e., (R³)ₚ). In some embodiments, p is 0 to 4 (i.e., 0, 1, 2, 3, or 4). In some embodiments, p is 0 (i.e., R¹ is unsubstituted). In some embodiments, p is 1. In some embodiments, p is 2. In some embodiments, p is 3. In some embodiments, p is 4.

As defined generally above, each R³ is independently halo, —S(O)₂—NR^{3a}R^{3b}, —S(O)₂—R^{3b}, —S(NR^{3c})(O)—NR^{3a}R^{3b}, —S(O)(NR^{3c})—R^{3b}, —S(O)—R^{3b}, —NR^{3a}S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S.

In some embodiments, each R³ is halo, oxo, —S(O)₂—NR^{3a}R^{3b}, or an optionally substituted group selected from C₁-C₆ aliphatic or C₃-C₁₂ cycloaliphatic.

In some embodiments, R³ is oxo.

In some embodiments, R³ is halo. In some embodiments, R³ is F, Cl, Br, or I. In some embodiments, R³ is F. In some embodiments, R³ is Cl. In some embodiments, R³ is Br. In some embodiments, R³ is I.

In some embodiments, R³ is —S(O)₂—NR^{3a}R^{3b}. In some embodiments, R³ is —S(O)₂—NH₂. In some embodiments, R; is —S(O)₂—NH(C₁-C₆ aliphatic). In some embodiments, R³ is —S(O)₂—N(C₁-C₆ aliphatic)₂. In some embodiments, R³ is —S(O)₂—N(CH₃)₂.

In some embodiments, R³ is —S(O)₂—R^{b3}. In some embodiments, R³ is —S(O)₂—C₁-C₆ aliphatic. In some embodiments, R³ is —S(O)₂—CH₃. In some embodiments, R³ is —S(O)₂—CHF₂. In some embodiments, R³ is —S(O)₂-(3- to 12-membered heterocyclyl comprising 1 to 4 heteroatoms selected from N, O, and S). In some embodiments, R³ is —S(O)₂-(azetidinyl).

In some embodiments, R³ is —S(NH)(O)—NR^{3a}R^{3b}. In some embodiments, R³ is —S(NH)(O)—N(C₁₋₆ aliphatic)₂. In some embodiments, R is —S(NH)(O)—N(CH₃)₂. In some embodiments, R³ is —S(NH)(O)—NH(CH₃).

In some embodiments, R³ is —S(O)(NH)—R^{3b}. In some embodiments, R³ is —S(O)(NH)—C₁₋₆ aliphatic. In some embodiments, R³ is —S(O)(NH)—CH₃. In some embodiments, R³ is —S(O)(NH)—CHF₂.

In some embodiments, R³ is —S(O)—R^{3b}. In some embodiments, R³ is —S(O)—C₁₋₆ aliphatic. In some embodiments, R³ is —S(O)—CH₃.

In some embodiments, R³ is —NR^{3a}—S(O)₂—R^{3b}. In some embodiments, R³ is —NH—S(O)₂—C₁-C₆ aliphatic. In some embodiments, R³ is —NH—S(O)₂—CH₃. In some embodiments, R³ is —N(C₁-C₆ aliphatic)-S(O)₂—C₁-C₆ aliphatic. In some embodiments, R³ is —N(CH₃)—S(O)₂—CH₃.

In some embodiments, R³ is —O—R^{3a}. In some embodiments, R³ is OH. In some embodiments, R³ is O—C₁-C₆ aliphatic, wherein C₁-C₆ aliphatic is optionally substituted with halo. In some embodiments, R³ is O—C₁-C₆ alkyl, wherein C₁-C₆ alkyl is optionally substituted with halo. In some embodiments, R³ is O—CH₃. In some embodiments, R³ is O—CH₂F. In some embodiments, R³ is O—CHF₂. In some embodiments, R³ is 0-CF₃.

In some embodiments, R³ is C(O)—R^{3a}. In some embodiments, R³ is C(O)—C₁-C₆ aliphatic. In some embodiments, R³ is C(O)—C₁-C₆ alkyl. In some embodiments, R³ is C(O)—CH₃.

In some embodiments, R³ is C(O)NH—R^{3a}. In some embodiments, R³ is C(O)NH—C₁-C₆ aliphatic. In some embodiments, R³ is C(O)NH—C₁-C₆ alkyl. In some embodiments, R³ is C(O)NH—CH₃.

In some embodiments, R³ is optionally substituted C₁-C₆ aliphatic. In some embodiments, R³ is C₁-C₆ aliphatic. In some embodiments, R³ is C₁-C₆ aliphatic substituted with substituted with one or more instances of halogen, —(CH₂)₀₋₄R^{∘}, —(CH₂)₀₋₄OR^{∘}, or —O(CH₂)₀₋₄R. In some embodiments, R³ is C₁-C₆ alkyl substituted with substituted with one or more instances of halogen, —(CH₂)₀₋₄R^{∘}, —(CH₂)₀₋₄OR^{∘}, or —O(CH₂)₀₋₄R^{∘}. In some embodiments, R³ is C₁-C₆ aliphatic substituted with substituted with one or more instances of halogen, —(CH₂)₀₋₄R^{∘}, —(CH₂)₀₋₄OR^{∘}, or —O(CH₂)₀₋₄R^{∘}, wherein each R^{∘} is independently, halo, C₁-C₆ aliphatic, C(O)—C₁-C₆ aliphatic, C(O)O—C₁-C₆ aliphatic or OH. In some embodiments, R³ methyl, ethyl, propyl, or butyl. In some embodiments, R³ is CH₂F. In some embodiments, R³ is CHF₂. In some embodiments, R³ is CF₃. In some embodiments, R³ is C(CH₃)₂—O—CH₃. In some embodiments, R³ is C(CH₃)₂—OH. In some embodiments, R³ is C(CH₃)₂—C(O)—CH₃.

In some embodiments, R³ is optionally substituted C₅-C₁₂ aryl. In some embodiments, R³ is C₅-C₁₂ aryl. In some embodiments, R³ is C₅-C₁₂ aryl substituted with —(CH₂)₀₋₄R^{∘}, —(CH₂)₀₋₄ OR^{∘}, or —O(CH₂)₀₋₄R^{∘}. In some embodiments, R³ is optionally substituted phenyl. In some embodiments, R³ is phenyl substituted with C₁-C₆ aliphatic. In some embodiments, R³ is phenyl substituted with CH₃.

In some embodiments, R³ is optionally substituted C₃-C₁₂ cycloaliphatic. In some embodiments, R³ is C₃-C₁₂ cycloaliphatic optionally substituted with halogen or C₁-C₆ aliphatic. In some embodiments, R³ is C₃-C₆ cycloaliphatic optionally substituted with halogen or C₁-C₆ aliphatic. In some embodiments, R³ is cyclopropyl, cyclobutyl, cyclopentyl, or cyclohexyl optionally substituted with halogen or C₁-C₆ aliphatic. In some embodiments, R³ is cyclopropyl. In some embodiments, R³ is cyclopropyl substituted with halogen.

In some embodiments, R³ is optionally substituted 5- to 12-membered heteroaryl comprising 1 to 3 heteroatoms selected from N, O, and S. In some embodiments, R³ is optionally substituted 5- to 6-membered heteroaryl. In some embodiments, R³ is 5- to 6-membered heteroaryl. In some embodiments, R³ is 5- to 6-membered heteroaryl optionally substituted with —(CH₂)₀₋₄R^{∘}, —(CH₂)₀₋₄OR^{∘}, or —O(CH₂)₀₋₄R^{∘}. In some embodiments, R³ is 5- to 6-membered heteroaryl optionally substituted with —(CH₂)₀₋₄R^{∘}, —(CH₂)₀₋₄OR^{∘}, or —O(CH₂)₀₋₄R^{∘}, wherein R^{∘} is C₁₋₆ aliphatic or a 3- to 6-membered saturated, partially unsaturated, or aryl ring having 0-4 heteroatoms independently selected from nitrogen, oxygen, or sulfur.

In some embodiments, R³ is optionally substituted 3- to 12-membered heterocyclyl comprising 1 to 3 heteroatoms selected from N, O, and S. In some embodiments, R³ is optionally substituted monocyclic 3- to 7-membered heterocyclyl comprising 1 to 3 heteroatoms selected from N, O, and S. In some embodiments, R³ is monocyclic 3- to 7-membered heterocyclyl comprising 1 to 3 heteroatoms selected from N, O, and S optionally substituted with halogen, —(CH₂)₀₋₄R^{∘}, —(CH₂)₀₋₄OR^{∘}, or —O(CH₂)₀₋₄R^{∘}. In some embodiments, R³ is monocyclic 3- to 7-membered heterocyclyl comprising 1 to 3 heteroatoms selected from N, O, and S optionally substituted with halogen, —(CH₂)₀₋₄R^{∘}, —(CH₂)₀₋₄OR^{∘}, or —O(CH₂)₀₋₄R^{∘}. In some embodiments, R³ is monocyclic 3- to 7-membered heterocyclyl comprising 1 to 3 heteroatoms selected from N, O, and S substituted with halogen, —(CH₂)₀₋₄R^{∘}, —(CH₂)₀₋₄OR^{∘}, or —O(CH₂)₀₋₄R^{∘}, where R^{∘} is C₁-C₆ aliphatic. In some embodiments, R³ is optionally substituted monocyclic 3-membered heterocyclyl comprising 1 to 3 heteroatoms selected from N, O, and S. In some embodiments, R³ is optionally substituted monocyclic 4-membered heterocyclyl comprising 1 to 3 heteroatoms selected from N, O, and S. In some embodiments, R³ is optionally substituted monocyclic 5-membered heterocyclyl comprising 1 to 3 heteroatoms selected from N, O, and S. In some embodiments, R³ is optionally substituted monocyclic 6-membered heterocyclyl comprising 1 to 3 heteroatoms selected from N, O, and S. In some embodiments, R³ is optionally substituted monocyclic 7-membered heterocyclyl comprising 1 to 3 heteroatoms selected from N, O, and S. In some embodiments, R³ is dioxolanyl.

In some embodiments, R³ is optionally substituted 10- to 12-membered bicyclic heterocyclyl comprising 1 to 3 heteroatoms selected from N, O, and S. In some embodiments, R³ is optionally substituted 10- to 12-membered bicyclic heterocyclyl comprising 1 to 3 heteroatoms selected from N, O, and S optionally substituted with halogen, oxo, —(CH₂)₀₋₄R^{∘}, —(CH₂)₀₋₄OR^{∘}, or —O(CH₂)₀₋₄R^{∘}. In some embodiments, R³ is optionally substituted 10- to 12-membered bicyclic heterocyclyl comprising 1 to 3 heteroatoms selected from N, O, and S optionally substituted with halogen, oxo, —(CH₂)₀₋₄R^{∘}, —(CH₂)₀₋₄OR^{∘}, or —O(CH₂)₀₋₄R, where R^{∘} is C₁-C₆ aliphatic. In some embodiments, R³ is optionally substituted 10-membered bicyclic heterocyclyl comprising 1 to 3 heteroatoms selected from N, O, and S optionally substituted with halogen, oxo, —(CH₂)₀₋₄R^{∘}, —(CH₂)₀₋₄OR^{∘}, or —O(CH₂)₀₋₄R^{∘}, where R^{∘} is C₁-C₆ aliphatic. In some embodiments, R³ is optionally substituted 11-membered bicyclic heterocyclyl comprising 1 to 3 heteroatoms selected from N, O, and S optionally substituted with halogen, oxo, —(CH₂)₀₋₄R^{∘}, —(CH₂)₀₋₄OR^{∘}, or —O(CH₂)₀₋₄R^{∘}, where R^{∘} is C₁-C₆ aliphatic. In some embodiments, R³ is optionally substituted 12-membered bicyclic heterocyclyl comprising 1 to 3 heteroatoms selected from N, O, and S optionally substituted with halogen, oxo, —(CH₂)₀₋₄R^{∘}, —(CH₂)₀₋₄OR^{∘}, or —O(CH₂)₀₋₄R^{∘}, where R^{∘} is C₁-C₆ aliphatic.

As defined generally above, R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S. In some embodiments, R^{3a} and R^{3b} are each independently selected from H and optionally substituted C₁-C₆ aliphatic. In some embodiments, R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S.

As defined generally above, each R^{c} is independently selected from H, OH, and optionally substituted C₁-C₆ aliphatic. In some embodiments, R^{3c} is H. In some embodiments, R^{3c} is OH. In some embodiments, R^{3c} is optionally substituted C₁-C₆ aliphatic. In some embodiments, R^{3c} is CH₃.

In some embodiments, R¹ is selected from: —CH(CH₃)₂,

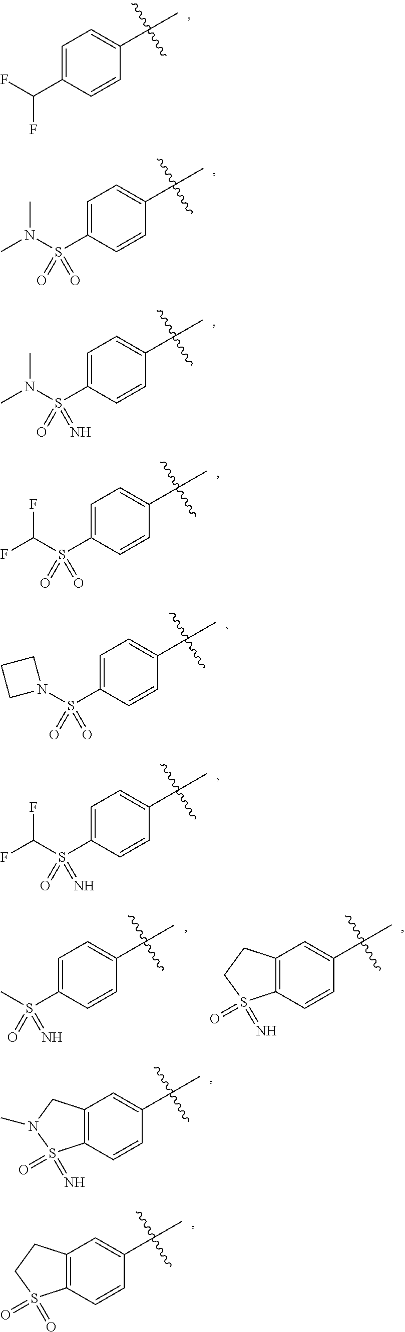

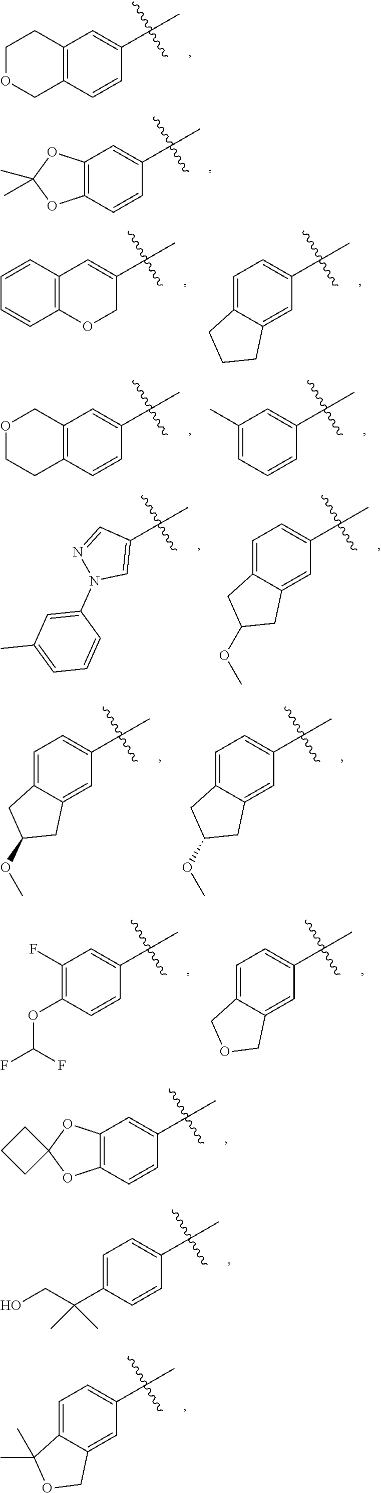

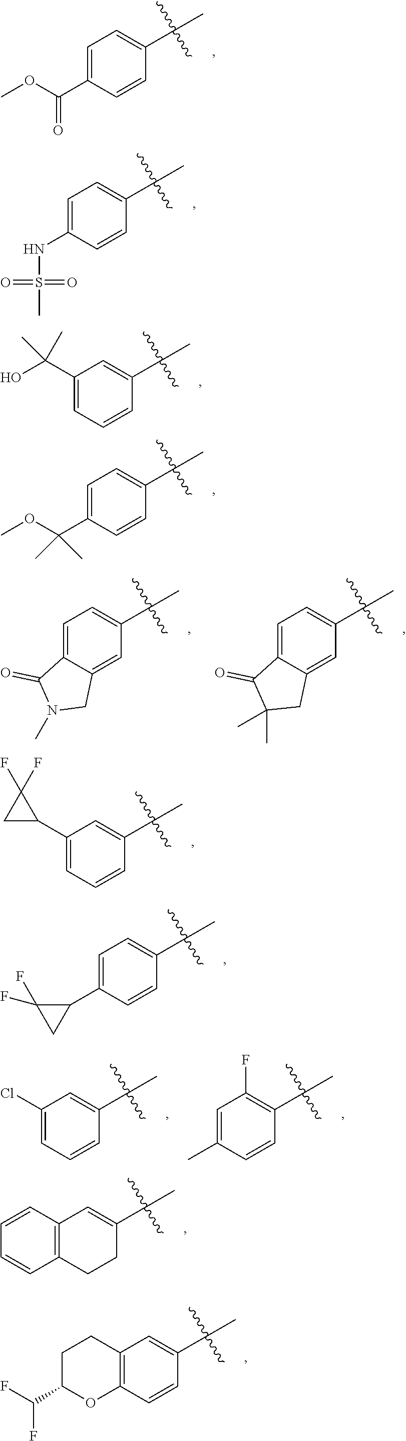

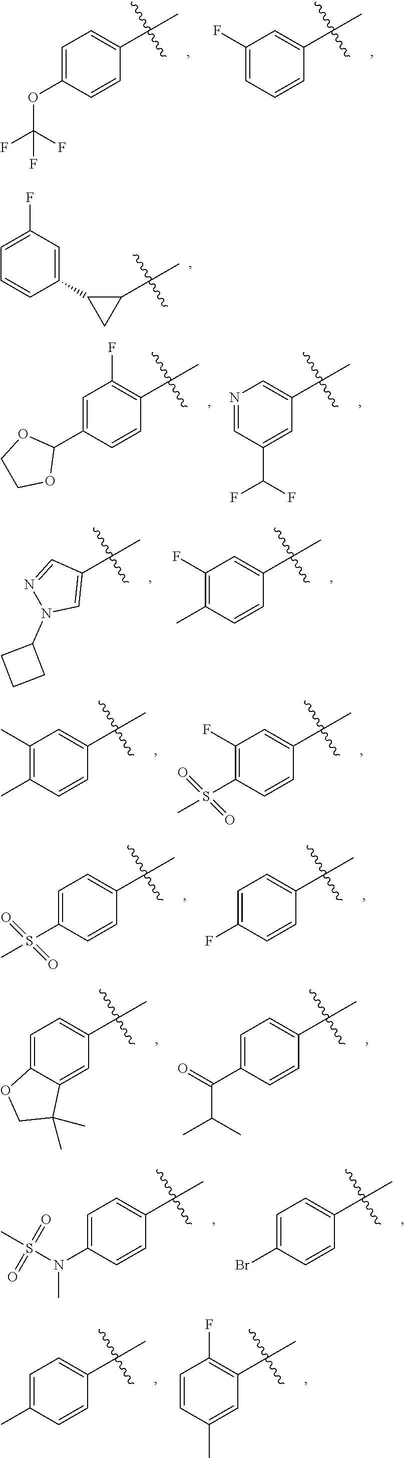

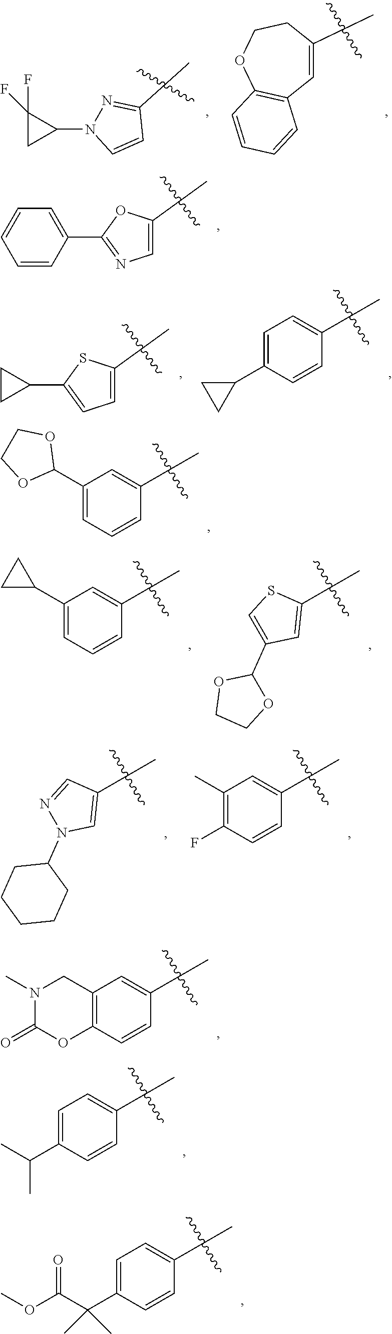

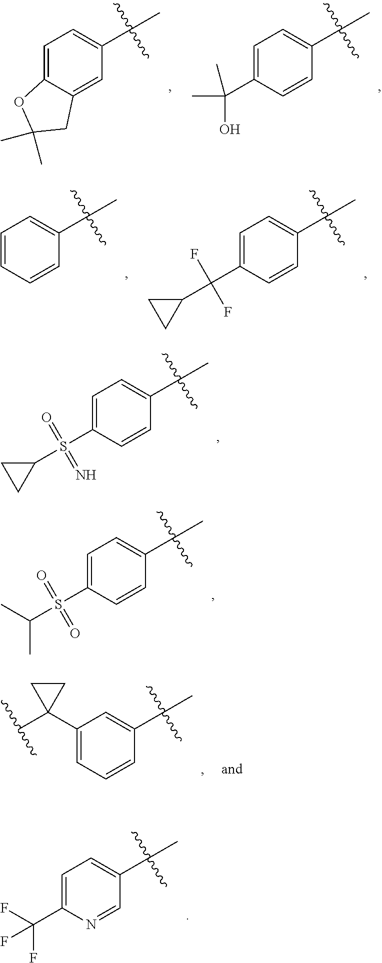

As defined generally above, each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S.

In some embodiments, each R² is independently halo, oxo, —NR^{2a}R^{2b}, —C(O)O—R^{2a}, —C(O)—NR^{2a}R^{2b}, —N(R^{2a})—C(O)—R^{2b}, —C(O)—R²³, —O—R^{2a}, or an optionally substituted group selected from C₁-C₆ aliphatic, C₅-C₁₂ aryl, and 3- to 12-membered heterocyclyl comprising 1 to 3 heteroatoms selected from N, O, and S.

In some embodiments, R² is halo. In some embodiments, R² is F, Br, Cl, or I. In some embodiments, R² is F. In some embodiments, R² is Br. In some embodiments, R² is Cl.

In some embodiments, R² is oxo.

In some embodiments, R² is —N(R^{2a})(R^{2b}). In some embodiments, R² is —NH(R^{2b}). In some embodiments, R² is —NH(R^{2a}). In some embodiments, R² is —NH₂.

In some embodiments, R² is —C(O)O—R^{2a}. In some embodiments, R² is —C(O)OH. In some embodiments, R² is —C(O)O—C₁-C₆ aliphatic. In some embodiments, R² is —C(O)O—C₁-C₆ aliphatic optionally substituted with —(CH₂)₀₋₄R^{∘}, —(CH₂)₀₋₄OR^{∘}, or —O(CH₂)₀₋₄R^{∘}. In some embodiments, R² is —C(O)O—C₁-C₆ aliphatic optionally substituted with —(CH₂)₀₋₄R^{∘}. In some embodiments R² is —C(O)O—C₁-C₆ aliphatic optionally substituted with —(CH₂)₀₋₄R^{∘}, where R^{∘} is an optionally substituted C₁₋₆ aliphatic or an optionally substituted 3- to 6-membered saturated, partially unsaturated, or aryl ring having 0-4 heteroatoms independently selected from nitrogen, oxygen, or sulfur. In some embodiments R² is —C(O)O—C₁-C₆ aliphatic optionally substituted with —(CH₂)₀₋₄R^{∘}, where R^{∘} is C₁—, aliphatic or a 3- to 6-membered saturated, partially unsaturated, or aryl ring having 0-4 heteroatoms independently selected from nitrogen, oxygen, or sulfur, where R^{∘} is substituted with —(CH₂)₀₋₂R^{•}, where R^{•} is C₁-C₆ aliphatic. In some embodiments, R² is —C(O)O—C₁-C₆ aliphatic optionally substituted with phenyl. In some embodiments, R² is —C(O)O—C₁-C₆ aliphatic optionally substituted with pyridyl. In some embodiments, R² is —C(O)O—C₁-C₆ aliphatic optionally substituted with pyrazolyl. In some embodiments, R² is —C(O)O—C₁-C₆ aliphatic optionally substituted with pyrazolyl substituted with —(CH₂)₀₋₂R^{•}. In some embodiments, R² is —C(O)O—C₁-C₆ aliphatic optionally substituted with imidazolyl. In some embodiments, R² is —C(O)O—C₁-C₆ aliphatic optionally substituted with imidazolyl substituted with —(CH₂)₀₋₂R^{•}. In some embodiments, R² is —C(O)O—C₁-C₆ aliphatic optionally substituted with isoxazolyl. In some embodiments, R² is —C(O)O—C₁-C₆ aliphatic optionally substituted with C₃-C₆ cycloalkyl. In some embodiments, R² is —C(O)O—C₁-C₆ aliphatic optionally substituted with cyclopropyl. In some embodiments, R² is —C(O)O—CH(CH₃)₂. In some embodiments, R² is —C(O)O—C(CH₃)₃. In some embodiments, R² is —C(O)O—R^{2a}, where R^{2a} is optionally substituted C₅-C₁₄ aryl. In some embodiments, R² is —C(O)O-phenyl.

In some embodiments, R² is —C(O)—N(R^{2a})(R^{2b}). In some embodiments, R² is —C(O)—N(H)(R^{2b}). In some embodiments, R² is —C(O)—N(C₁-C₆ aliphatic)(R^{2b}). In some embodiments, R² is —C(O)—N(R^{2a})(R^{2b}), where R^{2b} is optionally substituted C₁-C₆ aliphatic. In some embodiments R², is —C(O)—N(R^{2a})(R^{2b}), where R^{2b} is C₁-C₆ aliphatic optionally substituted with halogen, —(CH₂)₀₋₄R^{∘}, or —(CH₂)₀₋₄OR^{∘}. In some embodiments R² is —C(O)—N(R^{2a})(R^{2b}), where R^{2b} is C₁-C₆ aliphatic optionally substituted with —(CH₂)₀₋₄R^{∘}, where R^{∘} is selected from hydrogen and 3- to 6-membered saturated, partially unsaturated, or aryl ring having 0-4 heteroatoms independently selected from nitrogen, oxygen, or sulfur. °. In some embodiments R² is —C(O)—N(R²)(R^{2b}) where R^{b} is C₁-C₆ aliphatic optionally substituted with a group selected from cyclopropyl, phenyl, tetrahydrofuranyl, pyrazolyl, isoxazolyl, tetrahydropyranyl, and pyridyl. In some embodiments R² is —C(O)—N(R^{2a})(R^{2b}), where R^{2b} is C₁-C₆ aliphatic optionally substituted with —(CH₂)₀₋₄OR^{∘}, where R^{∘} is C₁₋₆ aliphatic. In some embodiments, R² is —C(O)—N(H)(CH₂CHF₂). In some embodiments, R² is —C(O)—N(H)(CH₂CF₃). In some embodiments, R² is —C(O)—N(H)(CH₂CH₂CF₃). In some embodiments, R² is —C(O)—N(H)(CH(CH₃)₂). In some embodiments R² is —C(O)—N(R^{2a})(R^{2b}), where R^{2b} is optionally substituted C₃-C₁₂ cycloaliphatic. In some embodiments R² is —C(O)—N(R^{2a})(R^{2b}), where R^{2b} is optionally substituted C₃-C₆ cycloaliphatic. In some embodiments of R², R^{2b} is optionally substituted cyclopropyl. In some embodiments R² is —C(O)—N(R^{2a})(R^{2b}), where R^{2b} is optionally substituted cyclobutyl. In some embodiments R² is —C(O)—N(R^{2a})(R^{2b}), where R^{2b} is optionally substituted cyclohexyl. In some embodiments R² is —C(O)—N(R^{2a})(R^{2b}), where R^{2b} is optionally substituted C₅-C₁₄ aryl. In some embodiments R² is —C(O)—N(R^{2a})(R^{2b}), where R^{2b} is phenyl optionally substituted with —(CH₂)₀₋₄R^{∘} or —(CH₂)₀₋₄OR^{∘}. In some embodiments R² is —C(O)—N(R^{2a})(R^{2b}), where R^{b} is optionally substituted 5- to 12-membered heteroaryl comprising 1 to 4 heteroatoms selected from N, O, and S. In some embodiments R² is —C(O)—N(R^{2a})(R^{2b}), where R^{2b} is optionally substituted 5- to 6-membered heteroaryl comprising 1 to 2 heteroatoms selected from N, O, and S In some embodiments R² is —C(O)—N(R^{2a})(R^{2b}), where R^{2b} is selected from pyridinyl, pyrrolyl, pyrazolyl, imidazolyl, isothiazolyl, and isoxazolyl optionally substituted with halogen, —(CH₂)₀₋₄R^{∘}, or —(CH₂)₀₋₄OR^{∘}. In some embodiments R² is —C(O)—N(R^{2a})(R^{2b}), where R^{2b} is optionally substituted 3- to 12-membered heterocyclyl comprising 1 to 3 heteroatoms selected from N, O, and S. In some embodiments R² is —C(O)—N(R^{2a})(R^{2b}), where R^{2b} is optionally substituted tetrahydrofuranyl or tetrahydropyranyl. In some embodiments R² is —C(O)—N(R^{2a})(R^{2b}), where R^{2b} is an optionally substituted 9- to 10-membered bicyclic heterocyclyl comprising 1 to 3 heteroatoms selected from N, O, and S.

In some embodiments, R² is —N(R^{2a})—C(O)R^{2b}.

In some embodiments, R² is —O—C(O)R^{2a}.

In some embodiments, R² is —S(O)₂.

In some embodiments, R² is —S(O)₂—R^{2a}.

In some embodiments, R² is —O—C(O)—NR^{2a}R^{2b}.

In some embodiments, R² is —NH—C(O)—NR^{2a}R^{2b}.

In some embodiments, R² is —NH—C(O)—OR^{2a}.

In some embodiments, R² is —C₁-C₆ alkylenyl-C(O)—NR^{2a}R^{2b}.

In some embodiments, R² is —NH—S(O)₂—R^{2a}.

In some embodiments, R² is —C(O)R^{2a}. In some embodiments, R² is —C(O)—C₁-C₆ aliphatic. In some embodiments, R² is —C(O)—C₁-C₆ aliphatic optionally substituted with halogen, —(CH₂)₀₋₄R^{∘}, —(CH₂)₀₋₄OR^{∘}, or —O(CH₂)₀₋₄R^{∘}. In some embodiments, R² is —C(O)—C₁-C₆ aliphatic optionally substituted with —(CH₂)₀₋₄R^{∘} or —(CH₂)₀₋₄OR^{∘}, where R^{∘} is 3- to 6-membered saturated, partially unsaturated, or aryl ring having 0-4 heteroatoms independently selected from nitrogen, oxygen, or sulfur. In some embodiments, R² is —C(O)—C(CH₃)₃. In some embodiments, R² is —C(O)— C₃-C₁₂ cycloaliphatic. In some embodiments, R² is —C(O)— C₃-C₁₂ cyclopropyl. In some embodiments, R² is —C(O)—C₅-C₁₄ aryl. In some embodiments, R² is —C(O)-phenyl. In some embodiments, R² is —C(O)R^{2a}, where R^{2a} is a 3- to 12-membered heterocyclyl comprising 1 to 3 heteroatoms selected from N, O, and S. In some embodiments, R² is —C(O)R^{2a}, where R² is selected from optionally substituted pyrrolidinyl, piperidinyl, and morpholinyl. In some embodiments, R² is —C(O)R^{2′}, where R^{2′} is a 7- to 11-membered spirocyclic ring system spirocyclic ring system comprising 1 to 3 heteroatoms selected from N, O, and S.

In some embodiments, R² is —O—R^{2a}. In some embodiments, R² is —OH. In some embodiments, R² is —O—C₁-C₆ aliphatic. In some embodiments, R² is —O—C₁-C₆ aliphatic, optionally substituted with —(CH₂)₀₋₄R^{∘}, —(CH₂)₀₋₄OR^{∘}, or —O(CH₂)₀₋₄R^{∘}. In some embodiments, R² is —OCH₃.

In some embodiments, R² is optionally substituted C₁-C₆ aliphatic. In some embodiments, R² is optionally substituted C₁-C₆ alkyl. In some embodiments, R² is C₁-C₆ aliphatic optionally substituted with halo, —(CH₂)₀₋₄R^{∘}, —(CH₂)₀₋₄OR^{∘}, —O(CH₂)₀₋₄R^{∘}, or —(CH₂)₀₋₄N(R^{∘})₂. In some embodiments, R² is methyl, ethyl, propyl, or butyl. In some embodiments, R² is —CH₃. In some embodiments, R² is —(CH₂)₀₋₆—CF₃. In some embodiments, R² is —CH₂—CF₃. In some embodiments, R² is C₁-C₆ aliphatic substituted with —OR^{∘}, where R^{∘} is C₁-C₆ aliphatic or a 3- to 6-membered saturated, partially unsaturated, or aryl ring having 0-4 heteroatoms independently selected from nitrogen, oxygen, or sulfur. In some embodiments, R² is —CH₂—O—CH₃. In some embodiments, R² is —CH₂—O—CH₂CH₃. In some embodiments, R² is —CH₂—CF₂—CH₃. In some embodiments, R² is C₁-C₆ aliphatic optionally substituted with —(CH₂)₀₋₄N(R^{∘})₂. In some embodiments, R² is C₁-C₆ aliphatic optionally substituted with —(CH₂)₀₋₄N(R^{∘})₂, where R^{∘} is hydrogen, C₁₋₆ aliphatic or a 3- to 6-membered saturated, partially unsaturated, or aryl ring having 0-4 heteroatoms independently selected from nitrogen, oxygen, or sulfur. In some embodiments, R² is C₁-C₆ aliphatic optionally substituted with R^{∘}, where R^{∘} is C₁₋₆ aliphatic or a 3- to 6-membered saturated, partially unsaturated, or aryl ring having 0-4 heteroatoms independently selected from nitrogen, oxygen, or sulfur. In some embodiments, R² is C₁-C₆ aliphatic optionally substituted with R^{∘}, where R^{∘} is 3- to 6-membered saturated, partially unsaturated, or aryl ring having 0-4 heteroatoms independently selected from nitrogen, oxygen, or sulfur. In some embodiments, R² is C₁-C₆ aliphatic optionally substituted with R^{∘}, where R^{∘} is phenyl.

In some embodiments, R² is optionally substituted C₅-C₁₂ aryl. In some embodiments, R² is optionally substituted C₅-C₆ aryl. In some embodiments, R² is C₅-C₆ aryl optionally substituted with halo, —(CH₂)₀₋₄R^{∘}, —(CH₂)₀₋₄OR^{∘}, or —O(CH₂)₀₋₄R^{∘}. In some embodiments, R² is C₅-C₆ aryl substituted with halo. In some embodiments, R² is phenyl. In some embodiments, R² is phenyl substituted with halo.

In some embodiments, R² is 3- to 12-membered heterocyclyl comprising 1 to 3 heteroatoms selected from N, O, and S. In some embodiments, R² is optionally substituted 3- to 12-membered heterocyclyl comprising 1 to 3 heteroatoms selected from N, O, and S. In some embodiments, R² is optionally substituted monocyclic 3- to 7-membered heterocyclyl comprising 1 to 3 heteroatoms selected from N, O, and S. In some embodiments, R² is monocyclic 3- to 7-membered heterocyclyl comprising 1 to 3 heteroatoms selected from N, O, and S optionally substituted with halogen, —(CH₂)₀₋₄R^{∘}, —(CH₂)₀₋₄OR^{∘}, or —O(CH₂)₀₋₄R^{∘}. In some embodiments, R² is monocyclic 3- to 7-membered heterocyclyl comprising 1 to 3 heteroatoms selected from N, O, and S optionally substituted with halogen, —(CH₂)₀₋₄R^{∘}, —(CH₂)₀₋₄OR^{∘}, or —O(CH₂)₀₋₄R^{∘}. In some embodiments, R² is optionally substituted monocyclic 3-membered heterocyclyl comprising 1 to 3 heteroatoms selected from N, O, and S. In some embodiments, R² is optionally substituted monocyclic 4-membered heterocyclyl comprising 1 to 3 heteroatoms selected from N, O, and S. In some embodiments, R² is optionally substituted monocyclic 5-membered heterocyclyl comprising 1 to 3 heteroatoms selected from N, O, and S. In some embodiments, R² is optionally substituted monocyclic 6-membered heterocyclyl comprising 1 to 3 heteroatoms selected from N, O, and S. In some embodiments, R² is optionally substituted monocyclic 7-membered heterocyclyl comprising 1 to 3 heteroatoms selected from N, O, and S.

In some embodiments, each R² is selected from F, Br, C₁, CN, —OCH₃, —CH₂—CF₃, —CF₃, —NH₂, —CH₂—O—CH₃, —CH₂—O—CH₂CH₃, —CH₂—CF₂—CH₃, —CH₃, —OH, oxo,

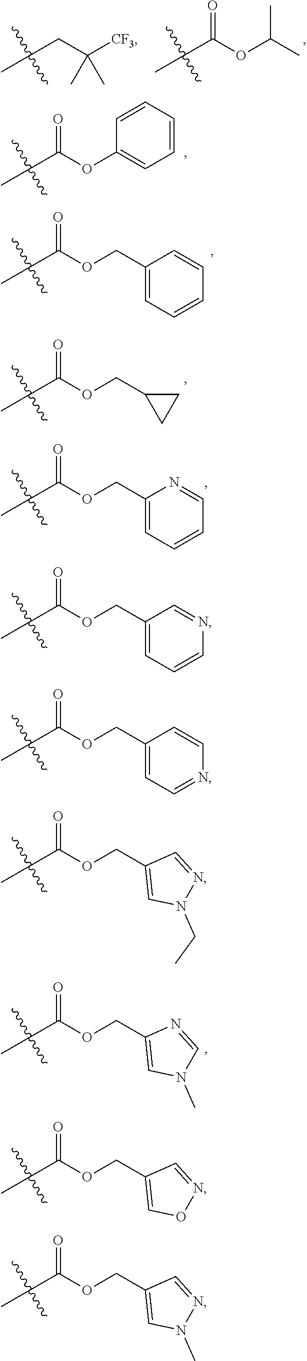

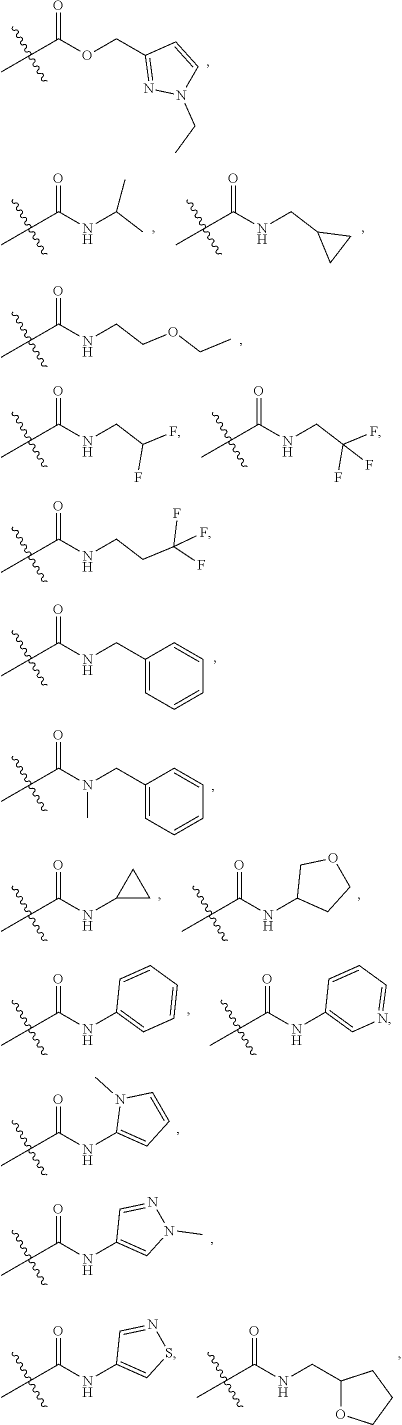

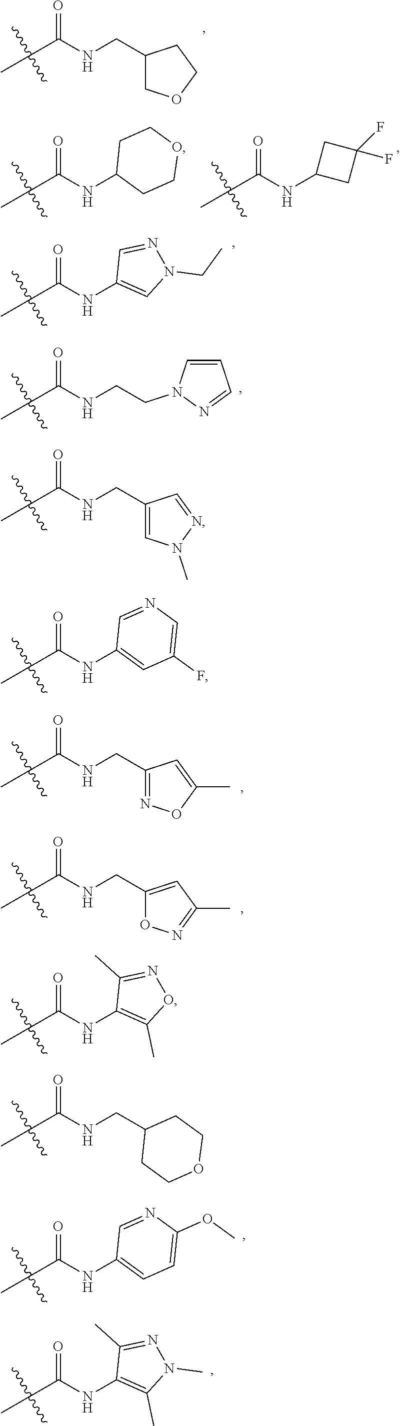

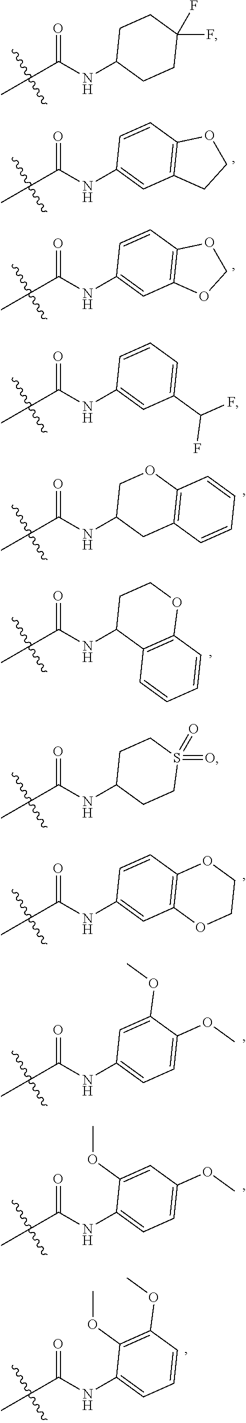

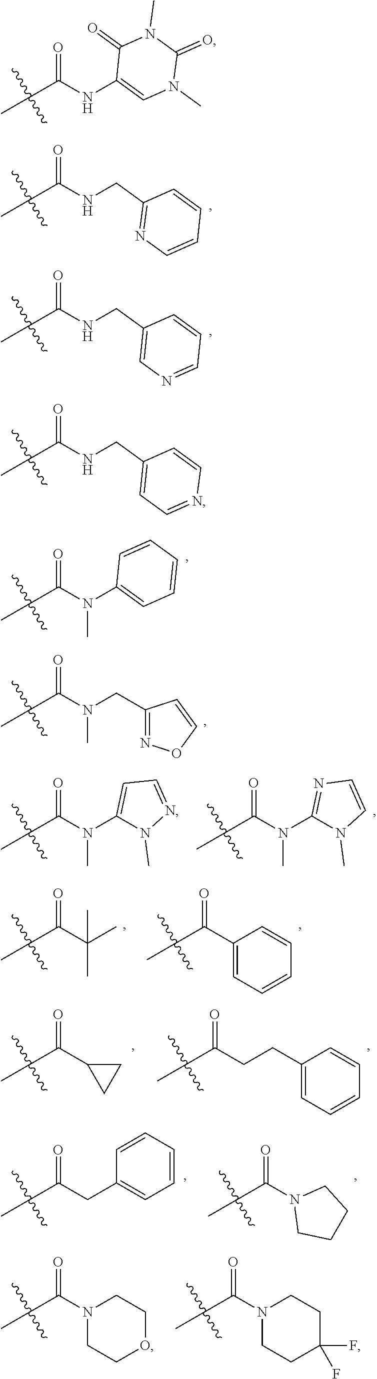

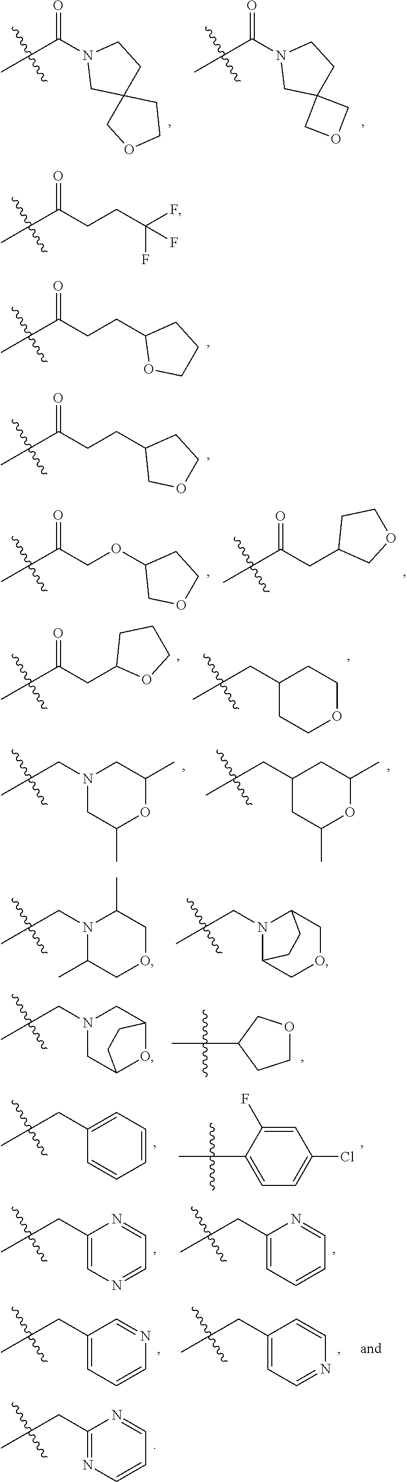

As defined generally above, each R^{2a} and each R^{2b} are independently selected from H and an optionally substituted group selected from C₁-C₆ aliphatic, C₃-C₁₂ cycloaliphatic, C₀-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S.

As defined generally above, each R⁵ is independently selected from hydrogen, halo, CN, and optionally substituted C₁-C₆ aliphatic. In some embodiments, R¹ is hydrogen. In some embodiments, R⁵ is halo. In some embodiments, R⁵ is CN. In some embodiments, R⁵ is optionally substituted C₁-C₆ aliphatic. In some embodiments, R⁸ is C₁-C₆ aliphatic optionally substituted with halogen, —(CH₂)₀₋₄R^{∘}, —(CH₂)₀₋₄OR^{∘}, or —O(CH₂)₀₋₄R^{∘}. In some embodiments, R⁵ is methyl, ethyl, propyl, or butyl. In some embodiments, R⁵ is methyl.

As defined generally above, R⁸ is selected from H and optionally substituted C₁-C₆ aliphatic. In some embodiments, R⁸ is H. In some embodiments, R⁸ is C₁-C₆ aliphatic. In some embodiments, R⁸ is methyl.

As defined generally above, n is 0 or 1. In some embodiments, n is 0. In some embodiments, n is 1.

As defined generally above, m is 0 to 4. In some embodiments, m is 0, 1, 2, 3, or 4. In some embodiments, m is 0. In some embodiments, m is 1. In some embodiments, m is 2. In some embodiments, m is 3. In some embodiments, m is 4.

As defined generally above, p is 0 to 4. In some embodiments, p is 0, 1, 2, 3, or 4. In some embodiments, p is 0. In some embodiments, p is 1. In some embodiments, p is 2. In some embodiments, p is 3. In some embodiments, p is 4.

As defined generally above, q is 1 or 2. In some embodiments, q is 1. In some embodiments, q is 2.

In some embodiments, q is 1 and n is 0 or 1. In some embodiments, q is 1 and n is 0, and a compound of formula I is:

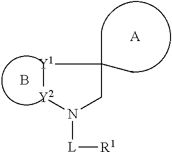

wherein R¹, L, Y¹, Y², A, and B are as defined herein and above.

In some embodiments, q is 1 and n is 1, and a compound of formula I is:

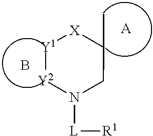

wherein R¹, L, Y¹, Y², X, A, and B are as defined herein and above.

In some embodiments, q is 2, and n is 0, and a compound of formula I is:

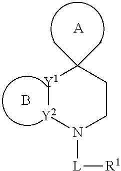

wherein R¹, L, Y¹, Y², A, and B are as defined herein and above.

As provided herein, the present disclosure provides and/or utilizes TRMPL1 modulators that are small molecule compounds having a chemical structure as indicated below in Formula Ia:

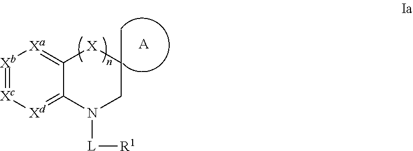

or a pharmaceutically acceptable salt thereof, wherein
X, n, A, L, and R¹ are as defined generally herein, and
each of X^{a}, X^{b}, X^{c}, and X^{d} are independently selected from N and CR⁶; and
each R⁶ is H, halo, CN, O—C₁-C₆ aliphatic, or an optionally substituted C₁-C₆ aliphatic.

In some embodiments, the present disclosure provides and/or utilizes TRMPL1 modulators that are small molecule compounds having a chemical structure as indicated below in Formula Ia:

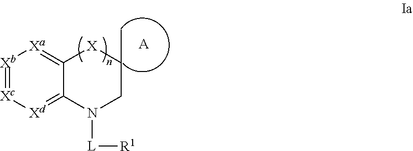

or a pharmaceutically acceptable salt thereof, wherein
X is —NR⁵—, —C(R⁵)₂—, —C(O)—, or —O—;
each of X^{a}, X^{b}, X^{c}, and X^{d} are independently selected from N and CR⁶;
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ:
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
each R³ is independently halo, —S(O)₂—NR^{3a}R^{3b}, —S(O)₂—R^{3b}, —S(O)(NR^{3c})—R^{3b}, —S(NR^{3c}O)—NR^{3a}R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, —OH, and optionally substituted C₁-C₆ aliphatic;
each R⁵ is independently selected from hydrogen, halo, —CN, and optionally substituted C₁-C₆ aliphatic;
R⁸ is selected from H and optionally substituted C₁-C₆ aliphatic;
each R⁶ is H, halo, CN, O—C₁-C₆ aliphatic, or an optionally substituted C₁-C₆ aliphatic;
n is 0 or 1,
m is 0 to 4; and
p is 0 to 4.

As described generally above, in some embodiments, each of X^{a}, X^{b}, X^{c}, and X^{d} are independently selected from N and CR⁶. In some embodiments, X^{a}, X^{b}, X^{c}, and X^{d} are each CR⁶. In some embodiments, X^{a}, X^{b}, X^{c}, and X^{d} are each CH. In some embodiments, X^{a}, X^{b}, and X^{c} are each CR⁶, and X^{d} is N. In some embodiments, X^{a}, X^{b}, and X^{d} are each CR⁶, and X^{c} is N. In some embodiments, X^{a}, X, and X^{d} are each CR⁶, and X^{b} is N. In some embodiments, X^{b}, X^{c}, and X^{d} are each CR⁶, and X^{a} is N. In some embodiments, X^{a} and X^{b}, are each CR⁶, and X^{c} and X^{d} are each N. In some embodiments, X^{a} and X^{c}, are each CR⁶, and X^{b} and X^{d} are each N. In some embodiments, X^{a} and X^{d}, are each CR⁶, and X^{b} and X^{c} are each N. In some embodiments, X^{b} and X^{c}, are each CR⁶, and X^{a} and X^{d} are each N. In some embodiments, X and X^{d}, are each CR⁶, and X^{a} and X^{b} are each N.

As described generally above, in some embodiments, each R⁶ is H, halo, CN, O—C₁-C₆ aliphatic, or an optionally substituted C₁-C₆ aliphatic. In some embodiments, R⁶ is H or halo. In some embodiments, R⁶ is H. In some embodiments, R⁶ is halo (e.g., fluoro, chloro, bromo, iodo). In some embodiments, R⁶ is CN. In some embodiments, R⁶ is O—C₁-C₆ aliphatic. In some embodiments, R⁶ is —OCH₃. In some embodiments, R⁶ is optionally substituted C₁-C₆ aliphatic. In some embodiments, R⁶ is C₁-C₆ aliphatic optionally substituted with halo. In some embodiments, R⁶ is CH₃ or CF₃. In some embodiments, R⁶ is CH₃. In some embodiments, R⁶ is CF₃.

As provided herein, the present disclosure provides and/or utilizes TRMPL1 modulators that are small molecule compounds having a chemical structure as indicated below in Formula Ib:

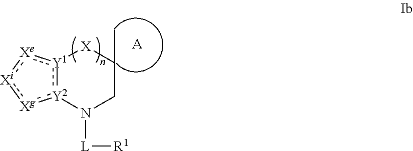

or a pharmaceutically acceptable salt thereof, wherein
X, n, A, L, R¹, Y¹, and Y² are as defined generally herein, and
each of X^{e}, X^{f}, and X^{g} are independently selected from S, N, O, and CR⁷; and
each R⁷ is H, halo, CN, O—C₁-C₆ aliphatic, or an optionally substituted group selected from C₁-C₆ aliphatic and C₃-C₆ cycloaliphatic.

In some embodiments, the present disclosure provides and/or utilizes TRMPL1 modulators that are small molecule compounds having a chemical structure as indicated below in Formula Ib:

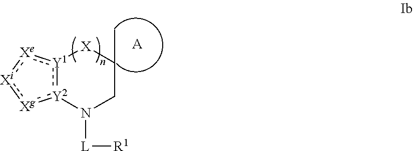

or a pharmaceutically acceptable salt thereof, wherein
X is —NR⁵—, —C(R⁵)₂—, —C(O)—, or —O—;
each of X^{e}, X^{f}, and X^{g} are independently selected from S, N, O, and CR⁷;
each of Y¹ and Y² is independently selected from N and C;
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR²R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S, each R^{2a} and each R^{2b} are independently selected from H and an optionally substituted group selected from C₁-C₆ aliphatic, C₃-C₁₂cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
each R³ is independently halo, —S(O)₂—NR^{3a}R^{3b}, —S(O)₂—R³⁵, —S(NR^{3c})(O)—NR^{3a}R^{3b}, —S(O)(NR^{3c})—R^{3b}, —S(O)—R^{3b}, —NR^{3a}, S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, —OH, and optionally substituted C₁-C₆ aliphatic;
each R⁵ is independently selected from hydrogen, halo, —CN, and optionally substituted C₁-C₆ aliphatic,
each R⁷ is H, halo, CN, O—C₁-C₆ aliphatic, or an optionally substituted group selected from C₁-C₆ aliphatic and C₃-C₆ cycloaliphatic;
R⁸ is selected from H and optionally substituted C₁-C₆ aliphatic;
n is 0 or 1;
m is 0 to 4; and p is 0 to 4.

As described generally above, in some embodiments, each of X^{e}, X^{f}, and X^{g} are independently selected from S, N, O, and CR⁷. In some embodiments, each of X^{e}, X^{f}, and X^{g} are CR⁷. In some embodiments, each of X^{e}, X^{f}, and X^{g} are independently selected from S and CR⁷. In some embodiments, each of X^{e}, X^{f}, and X^{g} are independently selected from N and CR⁷.

As described generally above, in some embodiments, each R⁷ is H, halo, CN, O—C₁-C₆ aliphatic, or an optionally substituted C₁-C₆ aliphatic. In some embodiments, R¹ is H or halo. In some embodiments, R⁷ is H. In some embodiments, R⁷ is halo (e.g., fluoro, chloro, bromo, iodo). In some embodiments, R⁷ is CN. In some embodiments, R⁷ is O—C₁-C₆ aliphatic. In some embodiments, R¹ is —OCH₃. In some embodiments, R⁷ is an optionally substituted group selected from C₁-C₆ aliphatic and C₃-C₆ cycloaliphatic. In some embodiments, R⁷ is optionally substituted C₁-C₆ aliphatic. In some embodiments, R¹ is C₁-C₆ aliphatic optionally substituted with halo. In some embodiments, R⁷ is CH₃ or CF₃. In some embodiments, R⁷ is CH₃. In some embodiments, R¹ is CF₃. In some embodiments, R⁷ is optionally substituted C₃-C₆ cycloaliphatic.

In some embodiments, the present disclosure provides compounds of Formula Ic:

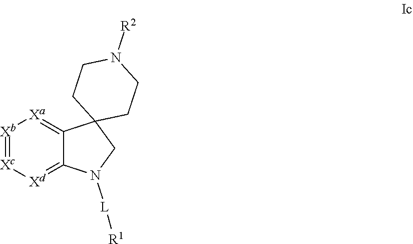

or a pharmaceutically acceptable salt thereof, wherein:
each of X^{a}, X^{b}, X^{c}, and X^{d} are independently selected from N and CR⁶; L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁵)—C₀-C₆ alkylenyl, —C₁-C₆ alkylenyl, and C₃-C₆ cycloalkylenyl;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2b}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
each R³ is independently halo, —S(O)₂—NR^{3a}R^{3b}, —S(O)₂—R³⁵, —S(O)(NR^{3c})—R^{3b}, —S(NR³)(O)—NR^{3a}R^{3b}, —S(O)—R^{3b}, —NR^{3a}, —S(O)₂—R^{3b}, —O—R^{3a}, —C(O)—R^{3a}, —C(O)NH—R^{3′}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R³ is independently selected from H, —OH, and optionally substituted C₁-C₆ aliphatic; R⁸ is selected from H and optionally substituted C₁-C₆ aliphatic;
each R⁶ is H, halo, CN, O—C₁-C₆ aliphatic, or an optionally substituted C₁-C₆ aliphatic; and
p is 0 to 4.

In some embodiments, the present disclosure provides compounds of Formula II:

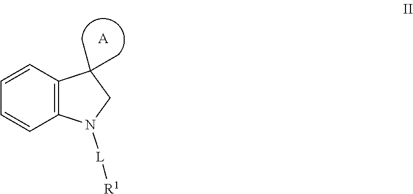

or a pharmaceutically acceptable salt thereof, wherein L, R¹, and A are as defined generally and herein.

In some embodiments, the present disclosure provides compounds of Formula IIa:

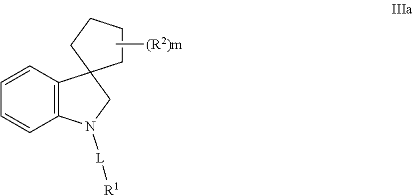

or a pharmaceutically acceptable salt thereof, wherein L, R¹, R², and m are as defined generally and herein.

In some embodiments, the present disclosure provides compounds of Formula IIIa:

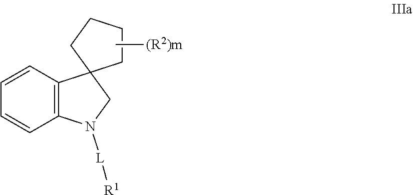

or a pharmaceutically acceptable salt thereof, wherein
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
R¹ is selected from C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R², —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3a})(O)—NR^{3a}R^{3b}, —S(O)(NR³C)—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, —OH, and optionally substituted C₁-C₆ aliphatic;
R⁸ is selected from H and optionally substituted C₁-C₆ aliphatic;
m is 0 to 4; and
p is 0 to 4.

In some embodiments, the present disclosure provides compounds of Formula IIIb:

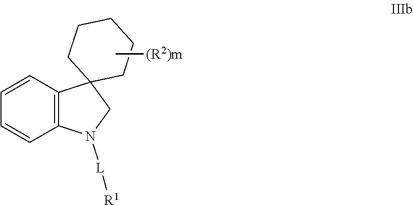

or a pharmaceutically acceptable salt thereof, wherein L, R¹, R², and m are as defined generally and herein.

In some embodiments, the present disclosure provides compounds of Formula IIIb:

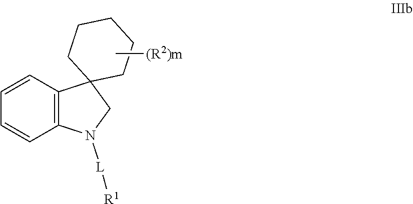

or a pharmaceutically acceptable salt thereof, wherein
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
R¹ is selected from C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3c})(O)—NR^{3a}R^{3b}, —S(O)(NR^{3c})—R^{3b}, —S(O)—R^{3b}, —NR^{3a}_S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S; R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, OH, and optionally substituted C₁-C₆ aliphatic;
R⁸ is selected from H and optionally substituted C₁-C₆ aliphatic;
m is 0 to 4; and
p is 0 to 4.

In some embodiments, the present disclosure provides compounds of Formula IVa:

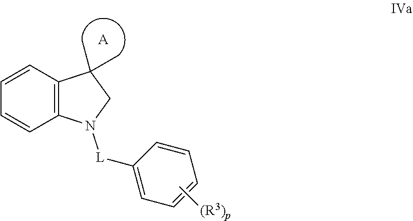

or a pharmaceutically acceptable salt thereof, wherein A, L, R³, and p are as defined generally and herein.

In some embodiments, the present disclosure provides compounds of Formula IVa:

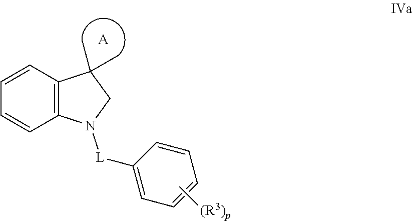

or a pharmaceutically acceptable salt thereof, wherein
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
each R² is independently halo, oxo, —NR^{2a}R^{2b}, —C(O)O—R²³, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3c})(O)—NR^{3a}R^{3b}, —S(O)(NR^{3c})—R^{3b}, —S(O)—R^{3b}, —NR^{3a}—S(O)₂—R^{3b}, —O—R^{3a}, —C(O)—R^{3a}, —C(O)NH—R 3a, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
R⁸ is selected from H and optionally substituted C₁-C₆ aliphatic;
m is 0 to 4; and
p is 0 to 4.

In some embodiments, the present disclosure provides compounds of Formula IVb:

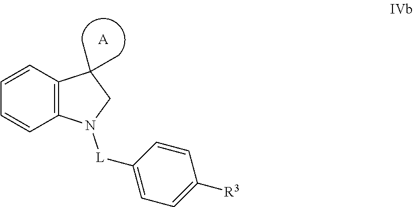

or a pharmaceutically acceptable salt thereof, wherein A, L, R³, and p are as defined generally and herein.

In some embodiments, the present disclosure provides compounds of Formula IVb:

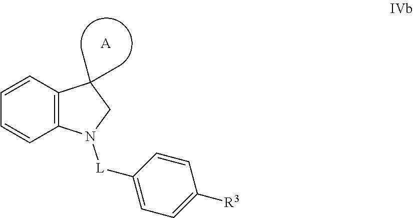

or a pharmaceutically acceptable salt thereof, wherein
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl,
A is C₃-C₁₂ cycloaliphatic or 3- to 12-membered heterocyclyl comprising 1 to 3 heteroatoms selected from N, O, and S, wherein A is substituted with (R²)ₘ;
each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R², —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3c}(O)—NR^{3a}R^{3b}, —S(O)(NR^{3c})—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
R⁸ is selected from H and optionally substituted C₁-C₆ aliphatic; and
m is 0 to 4.

In some embodiments, the present disclosure provides a compound, or a pharmaceutically acceptable salt thereof, selected from Table 1:

TABLE 1

| Compound | |
|---|---|
| No. | Structure |
| I-1 | 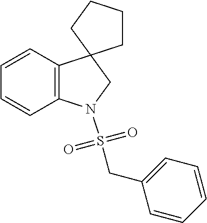 [SMILES: O=S(=O)(Cc1ccccc1)N1CC2(CCCC2)c2ccccc21] |
| I-2 | 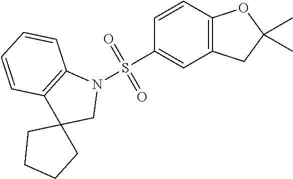 [SMILES: CC1(C)Cc2cc(S(=O)(=O)N3CC4(CCCC4)c4ccccc43)ccc2O1] |
| I-3 | 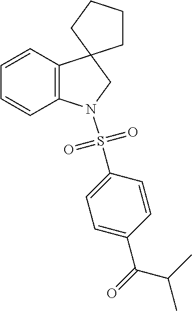 [SMILES: CC(C)C(=O)c1ccc(S(=O)(=O)N2CC3(CCCC3)c3ccccc32)cc1] |
| I-4 | 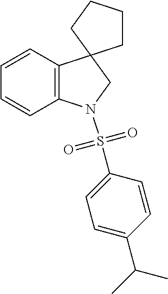 [SMILES: CC(C)c1ccc(S(=O)(=O)N2CC3(CCCC3)c3ccccc32)cc1] |
| I-5 | 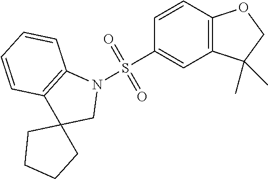 [SMILES: CC1(C)COc2ccc(S(=O)(=O)N3CC4(CCCC4)c4ccccc43)cc21] |
| I-6 | 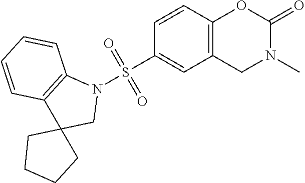 [SMILES: CN1Cc2cc(S(=O)(=O)N3CC4(CCCC4)c4ccccc43)ccc2OC1=O] |
| I-7 | 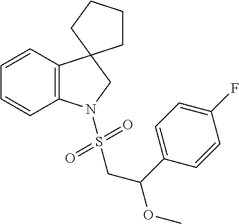 [SMILES: COC(CS(=O)(=O)N1CC2(CCCC2)c2ccccc21)c1ccc(F)cc1] |
| I-8 | 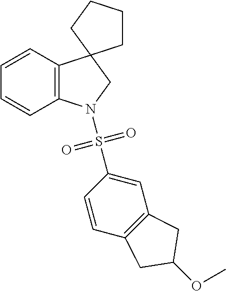 [SMILES: COC1Cc2ccc(S(=O)(=O)N3CC4(CCCC4)c4ccccc43)cc2C1] |
| I-9 | 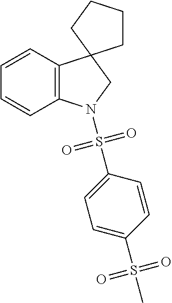 [SMILES: CS(=O)(=O)c1ccc(S(=O)(=O)N2CC3(CCCC3)c3ccccc32)cc1] |
| I-10 | 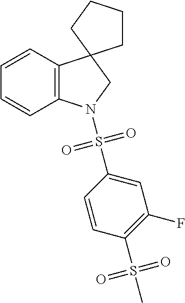 [SMILES: CS(=O)(=O)c1ccc(S(=O)(=O)N2CC3(CCCC3)c3ccccc32)cc1F] |
| I-11 | 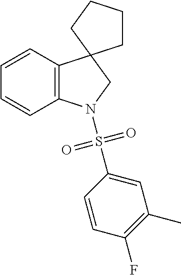 [SMILES: Cc1cc(S(=O)(=O)N2CC3(CCCC3)c3ccccc32)ccc1F] |
| I-12 | 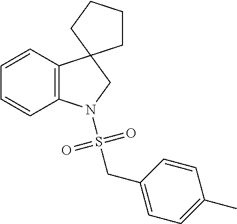 [SMILES: Cc1ccc(CS(=O)(=O)N2CC3(CCCC3)c3ccccc32)cc1] |
| I-13 | 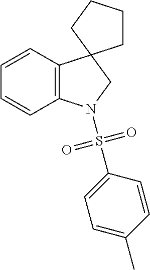 [SMILES: Cc1ccc(S(=O)(=O)N2CC3(CCCC3)c3ccccc32)cc1] |
| I-14 | 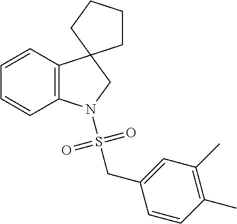 [SMILES: Cc1ccc(CS(=O)(=O)N2CC3(CCCC3)c3ccccc32)cc1C] |
| I-15 | 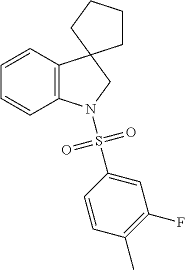 [SMILES: Cc1ccc(S(=O)(=O)N2CC3(CCCC3)c3ccccc32)cc1F] |
| I-16 | 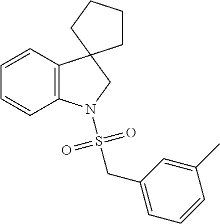 [SMILES: Cc1cccc(CS(=O)(=O)N2CC3(CCCC3)c3ccccc32)c1] |
| I-17 | 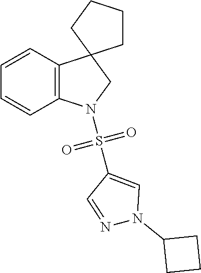 [SMILES: O=S(=O)(c1cnn(C2CCC2)c1)N1CC2(CCCC2)c2ccccc21] |
| I-18 | 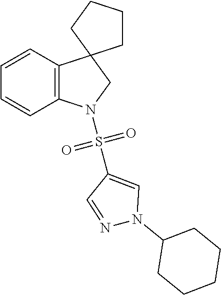 [SMILES: O=S(=O)(c1cnn(C2CCCCC2)c1)N1CC2(CCCC2)c2ccccc21] |
| I-19 | 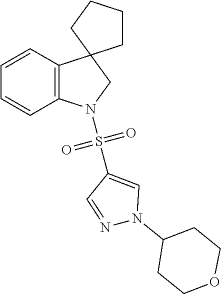 [SMILES: O=S(=O)(c1cnn(C2CCOCC2)c1)N1CC2(CCCC2)c2ccccc21] |
| I-20 | 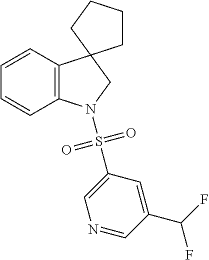 [SMILES: O=S(=O)(c1cncc(C(F)F)c1)N1CC2(CCCC2)c2ccccc21] |
| I-21 | 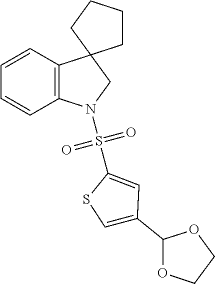 [SMILES: O=S(=O)(c1cc(C2OCCO2)cs1)N1CC2(CCCC2)c2ccccc21] |
| I-22 | 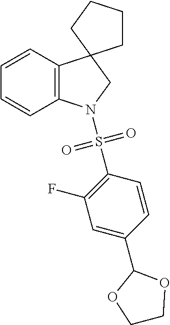 [SMILES: O=S(=O)(c1ccc(C2OCCO2)cc1F)N1CC2(CCCC2)c2ccccc21] |
| I-23 | 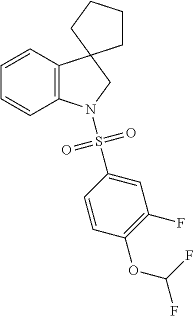 [SMILES: O=S(=O)(c1ccc(OC(F)F)c(F)c1)N1CC2(CCCC2)c2ccccc21] |
| I-24 | 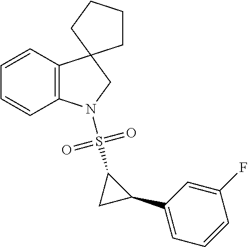 [SMILES: O=S(=O)([C@H]1C[C@@H]1c1cccc(F)c1)N1CC2(CCCC2)c2ccccc21] |
| I-25 | 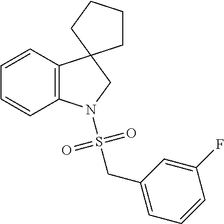 [SMILES: O=S(=O)(Cc1cccc(F)c1)N1CC2(CCCC2)c2ccccc21] |
| I-26 | 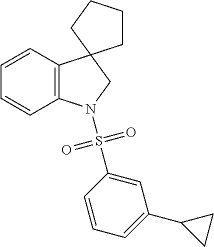 [SMILES: O=S(=O)(c1cccc(C2CC2)c1)N1CC2(CCCC2)c2ccccc21] |
| I-27 | 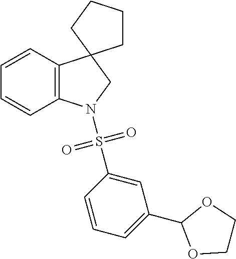 [SMILES: O=S(=O)(c1cccc(C2OCCO2)c1)N1CC2(CCCC2)c2ccccc21] |
| I-28 | 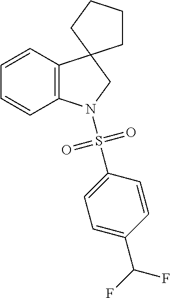 [SMILES: O=S(=O)(c1ccc(C(F)F)cc1)N1CC2(CCCC2)c2ccccc21] |
| I-29 | 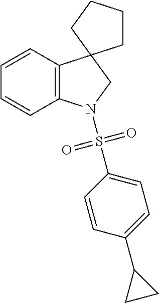 [SMILES: O=S(=O)(c1ccc(C2CC2)cc1)N1CC2(CCCC2)c2ccccc21] |
| I-30 | 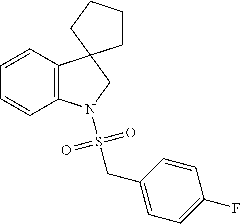 [SMILES: O=S(=O)(Cc1ccc(F)cc1)N1CC2(CCCC2)c2ccccc21] |
| I-31 | 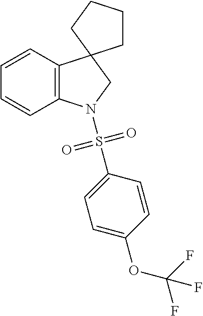 [SMILES: O=S(=O)(c1ccc(OC(F)(F)F)cc1)N1CC2(CCCC2)c2ccccc21] |
| I-32 | 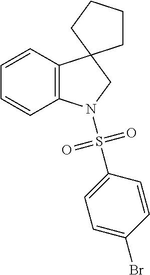 [SMILES: O=S(=O)(c1ccc(Br)cc1)N1CC2(CCCC2)c2ccccc21] |
| I-33 | 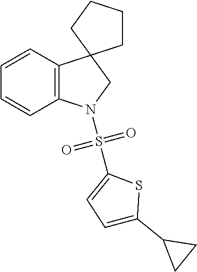 [SMILES: O=S(=O)(c1ccc(C2CC2)s1)N1CC2(CCCC2)c2ccccc21] |
| I-34 | 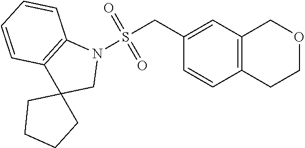 [SMILES: O=S(=O)(Cc1ccc2c(c1)COCC2)N1CC2(CCCC2)c2ccccc21] |
| I-35 | 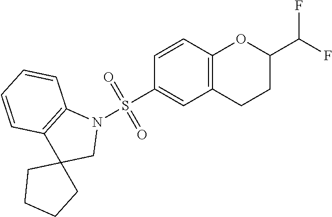 [SMILES: O=S(=O)(c1ccc2c(c1)CCC(C(F)F)O2)N1CC2(CCCC2)c2ccccc21] |
| I-36 | 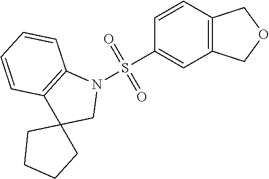 [SMILES: O=S(=O)(c1ccc2c(c1)COC2)N1CC2(CCCC2)c2ccccc21] |
| I-37 | 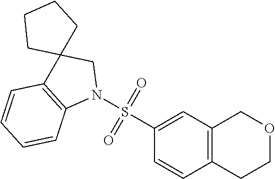 [SMILES: O=S(=O)(c1ccc2c(c1)COCC2)N1CC2(CCCC2)c2ccccc21] |
| I-38 | 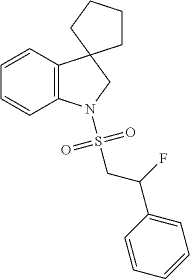 [SMILES: O=S(=O)(CC(F)c1ccccc1)N1CC2(CCCC2)c2ccccc21] |
| I-39 | 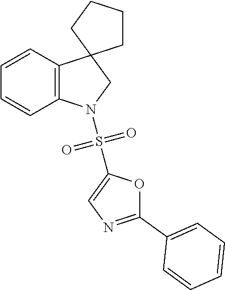 [SMILES: O=S(=O)(c1cnc(-c2ccccc2)o1)N1CC2(CCCC2)c2ccccc21] |
| I-40 | 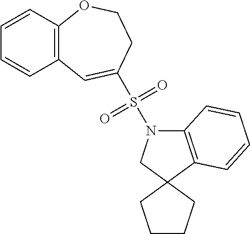 [SMILES: O=S(=O)(C1=Cc2ccccc2OCC1)N1CC2(CCCC2)c2ccccc21] |
| I-41 | 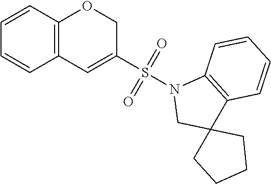 [SMILES: O=S(=O)(C1=Cc2ccccc2OC1)N1CC2(CCCC2)c2ccccc21] |
| I-42 | 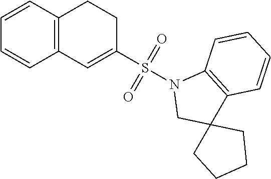 [SMILES: O=S(=O)(C1=Cc2ccccc2CC1)N1CC2(CCCC2)c2ccccc21] |
| I-43 | 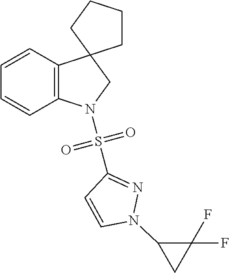 [SMILES: O=S(=O)(c1ccn(C2CC2(F)F)n1)N1CC2(CCCC2)c2ccccc21] |
| I-44 | 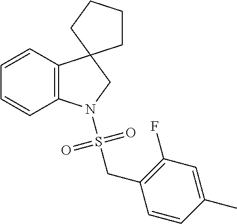 [SMILES: Cc1ccc(CS(=O)(=O)N2CC3(CCCC3)c3ccccc32)c(F)c1] |
| I-45 | 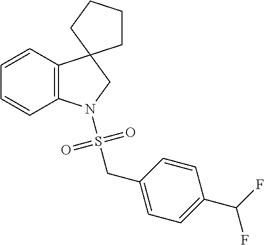 [SMILES: O=S(=O)(Cc1ccc(C(F)F)cc1)N1CC2(CCCC2)c2ccccc21] |
| I-46 | 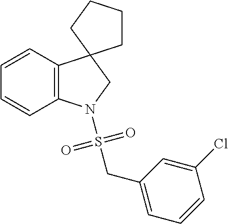 [SMILES: O=S(=O)(Cc1cccc(Cl)c1)N1CC2(CCCC2)c2ccccc21] |
| I-47 | 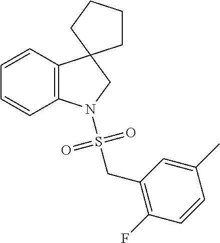 [SMILES: Cc1ccc(F)c(CS(=O)(=O)N2CC3(CCCC3)c3ccccc32)c1] |
| I-48 | 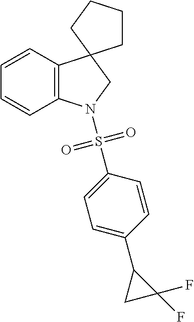 [SMILES: O=S(=O)(c1ccc(C2CC2(F)F)cc1)N1CC2(CCCC2)c2ccccc21] |
| I-49 | 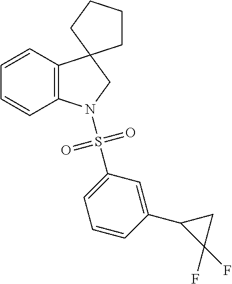 [SMILES: O=S(=O)(c1cccc(C2CC2(F)F)c1)N1CC2(CCCC2)c2ccccc21] |
| I-50 | 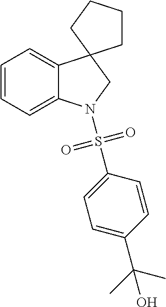 [SMILES: CC(C)(O)c1ccc(S(=O)(=O)N2CC3(CCCC3)c3ccccc32)cc1] |
| I-51 | 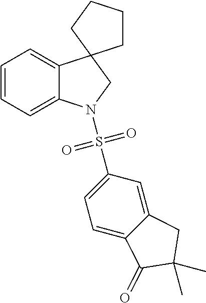 [SMILES: CC1(C)Cc2cc(S(=O)(=O)N3CC4(CCCC4)c4ccccc43)ccc2C1=O] |
| I-52 | 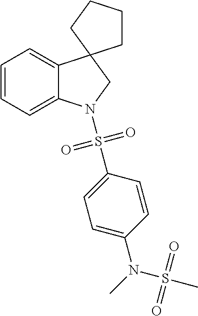 [SMILES: CN(c1ccc(S(=O)(=O)N2CC3(CCCC3)c3ccccc32)cc1)S(C)(=O)=O] |
| I-53 | 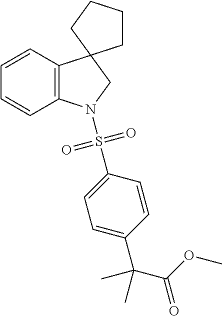 [SMILES: COC(=O)C(C)(C)c1ccc(S(=O)(=O)N2CC3(CCCC3)c3ccccc32)cc1] |
| I-54 | 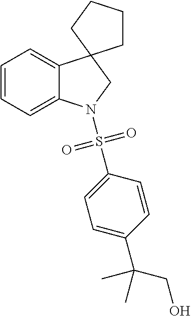 [SMILES: CC(C)(CO)c1ccc(S(=O)(=O)N2CC3(CCCC3)c3ccccc32)cc1] |
| I-55 | 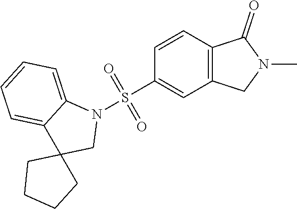 [SMILES: CN1Cc2cc(S(=O)(=O)N3CC4(CCCC4)c4ccccc43)ccc2C1=O] |
| I-56 | 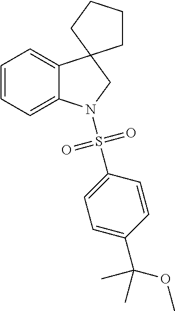 [SMILES: COC(C)(C)c1ccc(S(=O)(=O)N2CC3(CCCC3)c3ccccc32)cc1] |
| I-57 | 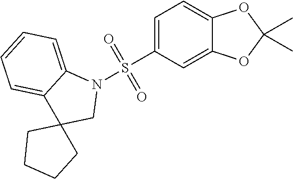 [SMILES: CC1(C)Oc2ccc(S(=O)(=O)N3CC4(CCCC4)c4ccccc43)cc2O1] |
| I-58 | 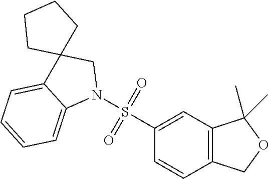 [SMILES: CC1(C)OCc2ccc(S(=O)(=O)N3CC4(CCCC4)c4ccccc43)cc21] |
| I-59 | 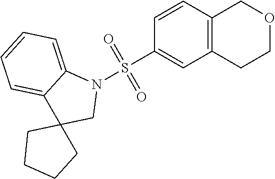 [SMILES: O=S(=O)(c1ccc2c(c1)CCOC2)N1CC2(CCCC2)c2ccccc21] |
| I-60 | 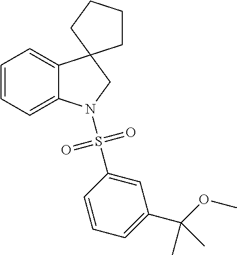 [SMILES: COC(C)(C)c1cccc(S(=O)(=O)N2CC3(CCCC3)c3ccccc32)c1] |
| I-61 | 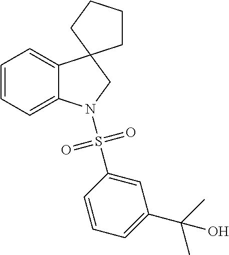 [SMILES: CC(C)(O)c1cccc(S(=O)(=O)N2CC3(CCCC3)c3ccccc32)c1] |
| I-62 | 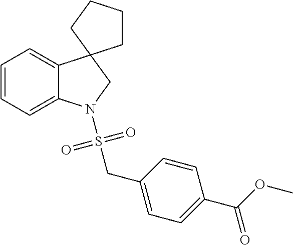 [SMILES: COC(=O)c1ccc(CS(=O)(=O)N2CC3(CCCC3)c3ccccc32)cc1] |
| I-63 | 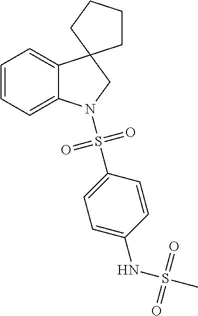 [SMILES: CS(=O)(=O)Nc1ccc(S(=O)(=O)N2CC3(CCCC3)c3ccccc32)cc1] |
| I-64 | 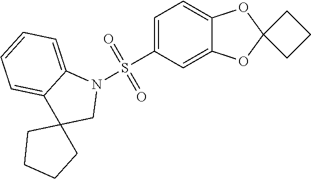 [SMILES: O=S(=O)(c1ccc2c(c1)OC1(CCC1)O2)N1CC2(CCCC2)c2ccccc21] |
| I-65 | 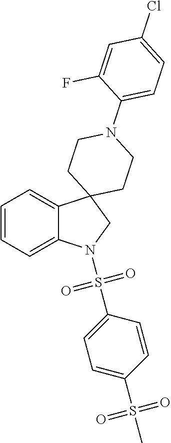 [SMILES: CS(=O)(=O)c1ccc(S(=O)(=O)N2CC3(CCN(c4ccc(Cl)cc4F)CC3)c3ccccc32)cc1] |
| I-66 | 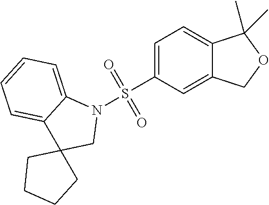 [SMILES: CC1(C)OCc2cc(S(=O)(=O)N3CC4(CCCC4)c4ccccc43)ccc21] |
| I-67 | 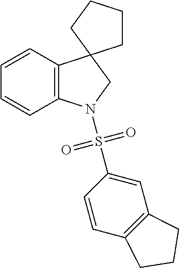 [SMILES: O=S(=O)(c1ccc2c(c1)CCC2)N1CC2(CCCC2)c2ccccc21] |
| I-68 | 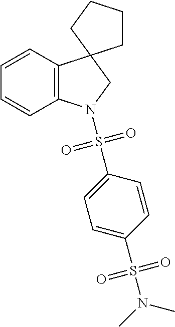 [SMILES: CN(C)S(=O)(=O)c1ccc(S(=O)(=O)N2CC3(CCCC3)c3ccccc32)cc1] |
| I-69 | 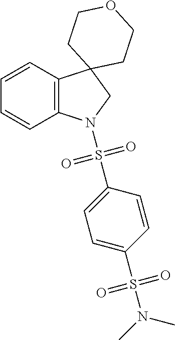 [SMILES: CN(C)S(=O)(=O)c1ccc(S(=O)(=O)N2CC3(CCOCC3)c3ccccc32)cc1] |
| I-70 | 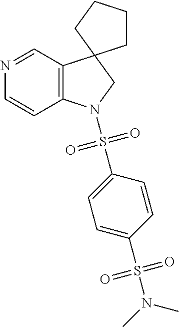 [SMILES: CN(C)S(=O)(=O)c1ccc(S(=O)(=O)N2CC3(CCCC3)c3cnccc32)cc1] |
| I-71 | 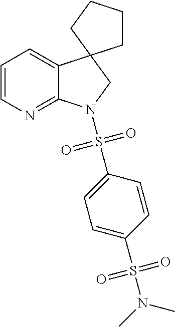 [SMILES: CN(C)S(=O)(=O)c1ccc(S(=O)(=O)N2CC3(CCCC3)c3cccnc32)cc1] |
| I-72 | 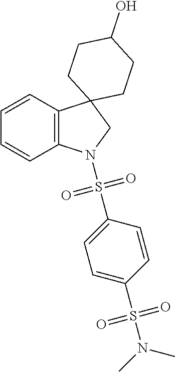 [SMILES: CN(C)S(=O)(=O)c1ccc(S(=O)(=O)N2CC3(CCC(O)CC3)c3ccccc32)cc1] |
| I-73 | 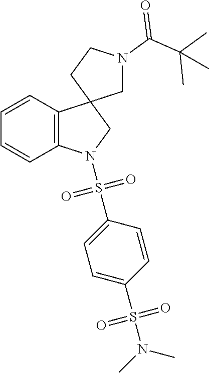 [SMILES: CN(C)S(=O)(=O)c1ccc(S(=O)(=O)N2CC3(CCN(C(=O)C(C)(C)C)C3)c3ccccc32)cc1] |
| I-74 | 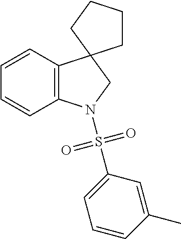 [SMILES: Cc1cccc(S(=O)(=O)N2CC3(CCCC3)c3ccccc32)c1] |
| I-75 | 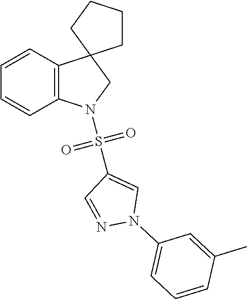 [SMILES: Cc1cccc(-n2cc(S(=O)(=O)N3CC4(CCCC4)c4ccccc43)cn2)c1] |
| I-76 | 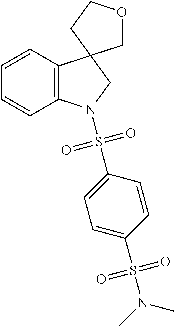 [SMILES: CN(C)S(=O)(=O)c1ccc(S(=O)(=O)N2CC3(CCOC3)c3ccccc32)cc1] |
| I-77 | 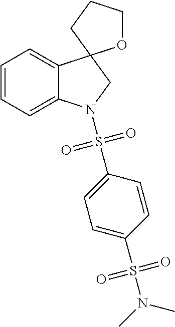 [SMILES: CN(C)S(=O)(=O)c1ccc(S(=O)(=O)N2CC3(CCCO3)c3ccccc32)cc1] |
| I-78 | 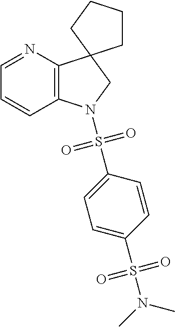 [SMILES: CN(C)S(=O)(=O)c1ccc(S(=O)(=O)N2CC3(CCCC3)c3ncccc32)cc1] |
| I-79 | 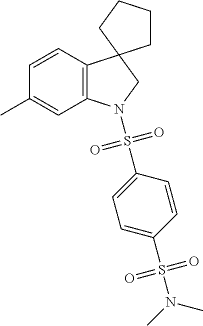 |
| I-80 | 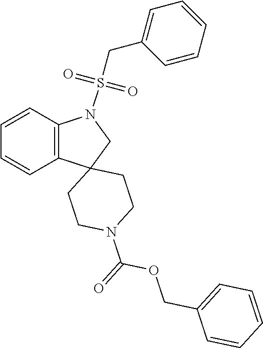 [SMILES: O=C(OCc1ccccc1)N1CCC2(CC1)CN(S(=O)(=O)Cc1ccccc1)c1ccccc12] |
| I-81 | 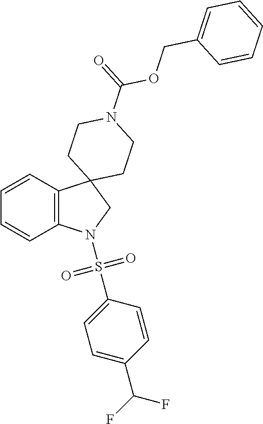 [SMILES: O=C(OCc1ccccc1)N1CCC2(CC1)CN(S(=O)(=O)c1ccc(C(F)F)cc1)c1ccccc12] |
| I-82 | 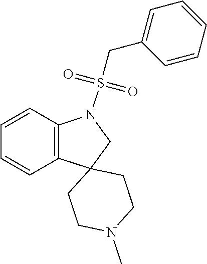 [SMILES: CN1CCC2(CC1)CN(S(=O)(=O)Cc1ccccc1)c1ccccc12] |
| I-83 | 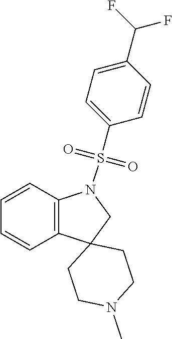 [SMILES: CN1CCC2(CC1)CN(S(=O)(=O)c1ccc(C(F)F)cc1)c1ccccc12] |
| I-84 | 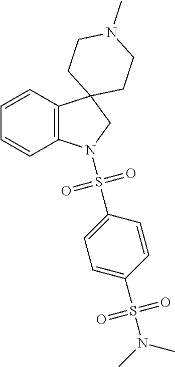 [SMILES: CN1CCC2(CC1)CN(S(=O)(=O)c1ccc(S(=O)(=O)N(C)C)cc1)c1ccccc12] |
| I-85 | 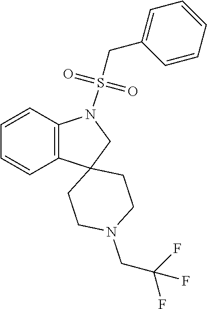 [SMILES: O=S(=O)(Cc1ccccc1)N1CC2(CCN(CC(F)(F)F)CC2)c2ccccc21] |
| I-86 | 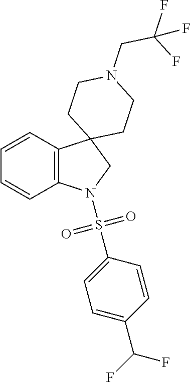 [SMILES: O=S(=O)(c1ccc(C(F)F)cc1)N1CC2(CCN(CC(F)(F)F)CC2)c2ccccc21] |
| I-87 | 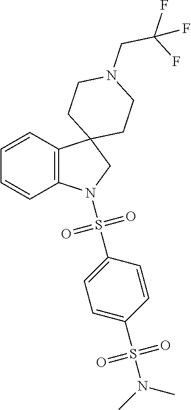 [SMILES: CN(C)S(=O)(=O)c1ccc(S(=O)(=O)N2CC3(CCN(CC(F)(F)F)CC3)c3ccccc32)cc1] |
| I-88 | 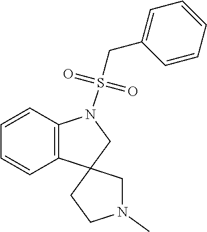 [SMILES: CN1CCC2(C1)CN(S(=O)(=O)Cc1ccccc1)c1ccccc12] |
| I-89 | 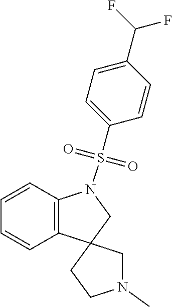 [SMILES: CN1CCC2(C1)CN(S(=O)(=O)c1ccc(C(F)F)cc1)c1ccccc12] |
| I-90 | 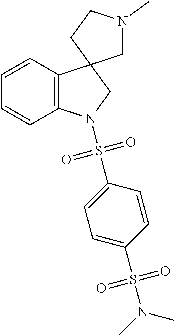 [SMILES: CN1CCC2(C1)CN(S(=O)(=O)c1ccc(S(=O)(=O)N(C)C)cc1)c1ccccc12] |
| I-91 | 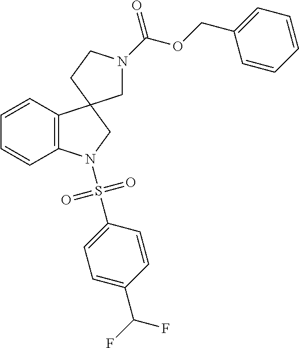 [SMILES: O=C(OCc1ccccc1)N1CCC2(C1)CN(S(=O)(=O)c1ccc(C(F)F)cc1)c1ccccc12] |
| I-92 | 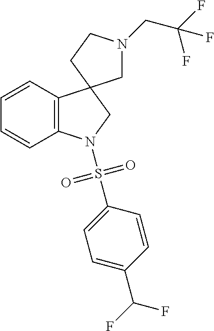 [SMILES: O=S(=O)(c1ccc(C(F)F)cc1)N1CC2(CCN(CC(F)(F)F)C2)c2ccccc21] |
| I-93 | 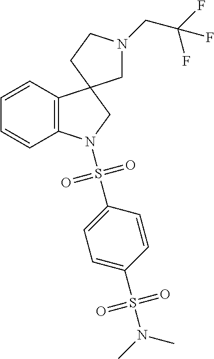 [SMILES: CN(C)S(=O)(=O)c1ccc(S(=O)(=O)N2CC3(CCN(CC(F)(F)F)C3)c3ccccc32)cc1] |
| I-94 | 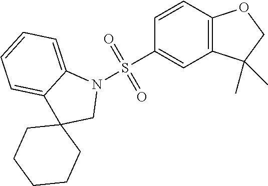 [SMILES: CC1(C)COc2ccc(S(=O)(=O)N3CC4(CCCCC4)c4ccccc43)cc21] |
| I-95 | 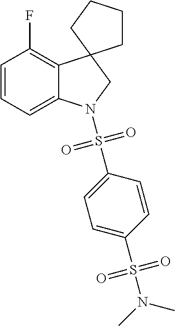 [SMILES: CN(C)S(=O)(=O)c1ccc(S(=O)(=O)N2CC3(CCCC3)c3c(F)cccc32)cc1] |
| I-96 | 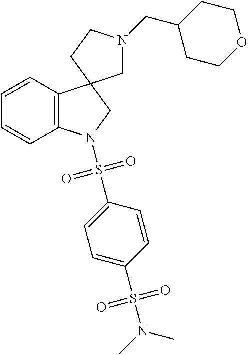 [SMILES: CN(C)S(=O)(=O)c1ccc(S(=O)(=O)N2CC3(CCN(CC4CCOCC4)C3)c3ccccc32)cc1] |
| I-97 | 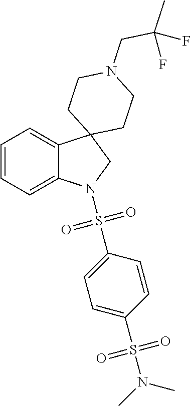 [SMILES: CN(C)S(=O)(=O)c1ccc(S(=O)(=O)N2CC3(CCN(CC(C)(F)F)CC3)c3ccccc32)cc1] |
| I-98 | 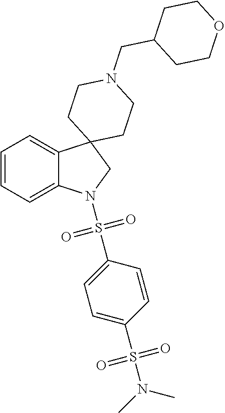 [SMILES: CN(C)S(=O)(=O)c1ccc(S(=O)(=O)N2CC3(CCN(CC4CCOCC4)CC3)c3ccccc32)cc1] |
| I-99 | 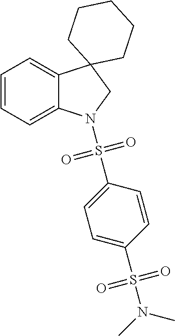 [SMILES: CN(C)S(=O)(=O)c1ccc(S(=O)(=O)N2CC3(CCCCC3)c3ccccc32)cc1] |
| I-100 | 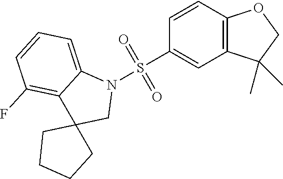 [SMILES: CC1(C)COc2ccc(S(=O)(=O)N3CC4(CCCC4)c4c(F)cccc43)cc21] |
| I-101 | 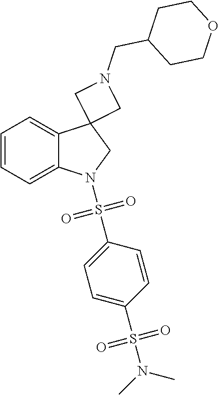 [SMILES: CN(C)S(=O)(=O)c1ccc(S(=O)(=O)N2CC3(CN(CC4CCOCC4)C3)c3ccccc32)cc1] |
| I-102 | 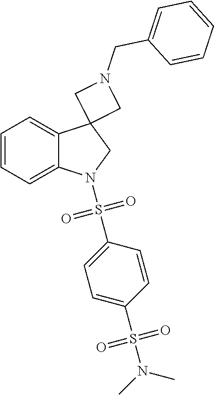 [SMILES: CN(C)S(=O)(=O)c1ccc(S(=O)(=O)N2CC3(CN(Cc4ccccc4)C3)c3ccccc32)cc1] |
| I-103 | 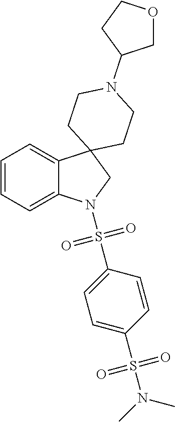 [SMILES: CN(C)S(=O)(=O)c1ccc(S(=O)(=O)N2CC3(CCN(C4CCOC4)CC3)c3ccccc32)cc1] |
| I-104 | 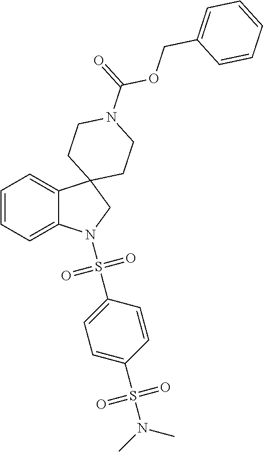 [SMILES: CN(C)S(=O)(=O)c1ccc(S(=O)(=O)N2CC3(CCN(C(=O)OCc4ccccc4)CC3)c3ccccc32)cc1] |
| I-105 | 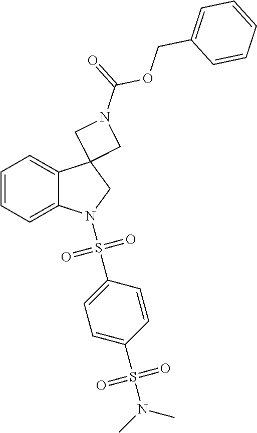 [SMILES: CN(C)S(=O)(=O)c1ccc(S(=O)(=O)N2CC3(CN(C(=O)OCc4ccccc4)C3)c3ccccc32)cc1] |
| I-106 | 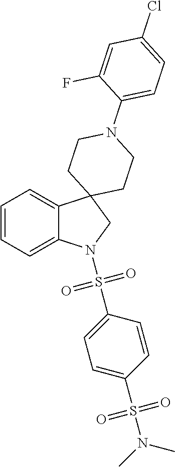 [SMILES: CN(C)S(=O)(=O)c1ccc(S(=O)(=O)N2CC3(CCN(c4ccc(Cl)cc4F)CC3)c3ccccc32)cc1] |
| I-114 | 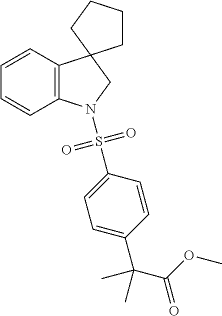 [SMILES: COC(=O)C(C)(C)c1ccc(S(=O)(=O)N2CC3(CCCC3)c3ccccc32)cc1] |
| I-115 | 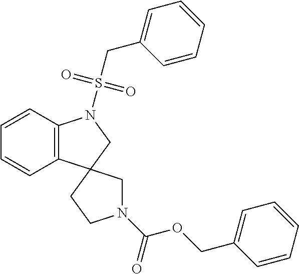 [SMILES: O=C(OCc1ccccc1)N1CCC2(C1)CN(S(=O)(=O)Cc1ccccc1)c1ccccc12] |
| I-116 | 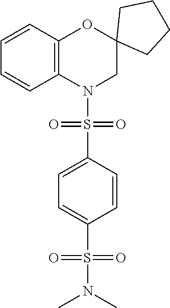 [SMILES: CN(C)S(=O)(=O)c1ccc(S(=O)(=O)N2CC3(CCCC3)Oc3ccccc32)cc1] |
| I-117 | 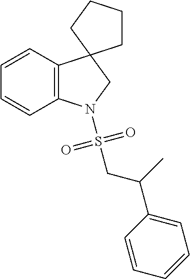 [SMILES: CC(CS(=O)(=O)N1CC2(CCCC2)c2ccccc21)c1ccccc1] |
| I-118 | 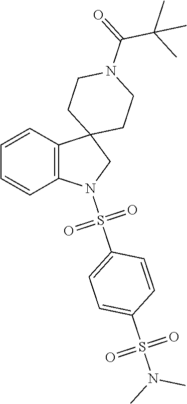 [SMILES: CN(C)S(=O)(=O)c1ccc(S(=O)(=O)N2CC3(CCN(C(=O)C(C)(C)C)CC3)c3ccccc32)cc1] |
| I-119 | 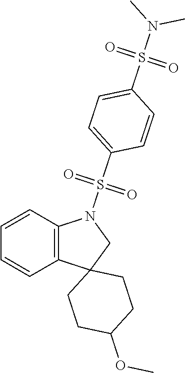 [SMILES: COC1CCC2(CC1)CN(S(=O)(=O)c1ccc(S(=O)(=O)N(C)C)cc1)c1ccccc12] |
| I-120 | 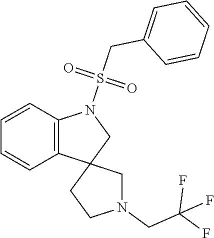 [SMILES: O=S(=O)(Cc1ccccc1)N1CC2(CCN(CC(F)(F)F)C2)c2ccccc21] |
| I-121 | 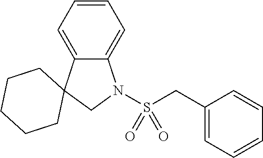 [SMILES: O=S(=O)(Cc1ccccc1)N1CC2(CCCCC2)c2ccccc21] |
| I-122 | 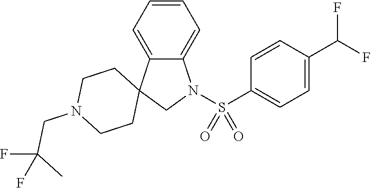 [SMILES: CC(F)(F)CN1CCC2(CC1)CN(S(=O)(=O)c1ccc(C(F)F)cc1)c1ccccc12] |
| I-123 | 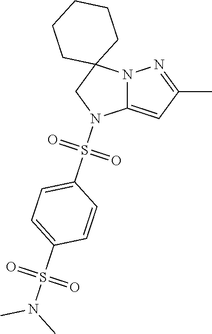 |
| I-124 | 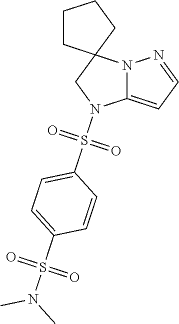 [SMILES: CN(C)S(=O)(=O)c1ccc(S(=O)(=O)N2CC3(CCCC3)n3nccc32)cc1] |
| I-125 | 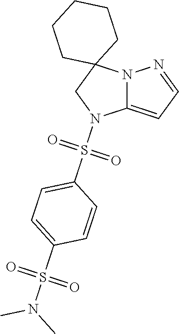 [SMILES: CN(C)S(=O)(=O)c1ccc(S(=O)(=O)N2CC3(CCCCC3)n3nccc32)cc1] |
| I-126 | 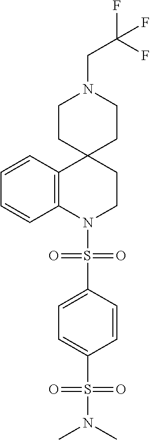 [SMILES: CN(C)S(=O)(=O)c1ccc(S(=O)(=O)N2CCC3(CCN(CC(F)(F)F)CC3)c3ccccc32)cc1] |
| I-127 | 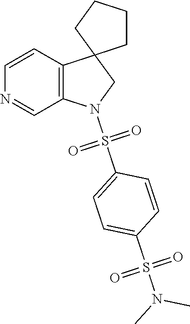 [SMILES: CN(C)S(=O)(=O)c1ccc(S(=O)(=O)N2CC3(CCCC3)c3ccncc32)cc1] |
| I-128 | 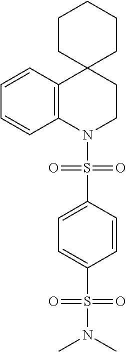 [SMILES: CN(C)S(=O)(=O)c1ccc(S(=O)(=O)N2CCC3(CCCCC3)c3ccccc32)cc1] |
| I-129 | 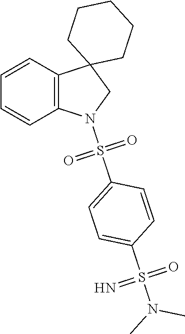 [SMILES: CN(C)S(=N)(=O)c1ccc(S(=O)(=O)N2CC3(CCCCC3)c3ccccc32)cc1] |
| I-130 | 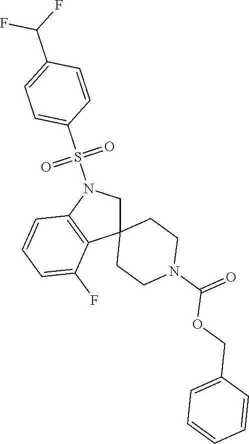 [SMILES: O=C(OCc1ccccc1)N1CCC2(CC1)CN(S(=O)(=O)c1ccc(C(F)F)cc1)c1cccc(F)c12] |
| I-131 | 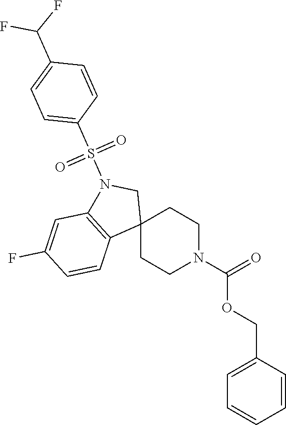 [SMILES: O=C(OCc1ccccc1)N1CCC2(CC1)CN(S(=O)(=O)c1ccc(C(F)F)cc1)c1cc(F)ccc12] |
| I-132 | 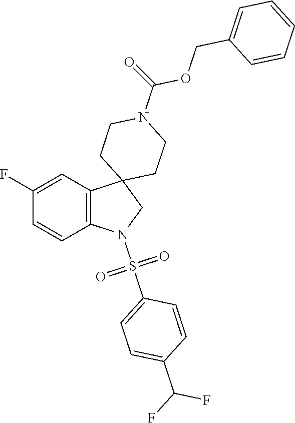 [SMILES: O=C(OCc1ccccc1)N1CCC2(CC1)CN(S(=O)(=O)c1ccc(C(F)F)cc1)c1ccc(F)cc12] |
| I-133 | 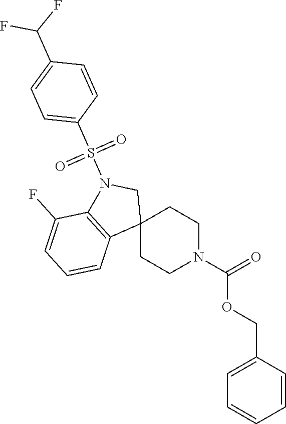 [SMILES: O=C(OCc1ccccc1)N1CCC2(CC1)CN(S(=O)(=O)c1ccc(C(F)F)cc1)c1c(F)cccc12] |
| I-134 | 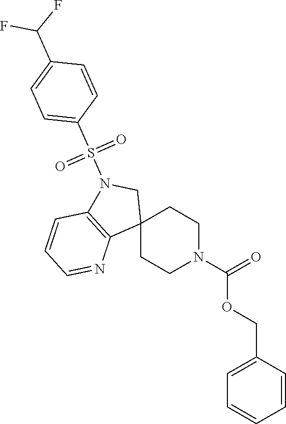 [SMILES: O=C(OCc1ccccc1)N1CCC2(CC1)CN(S(=O)(=O)c1ccc(C(F)F)cc1)c1cccnc12] |
| I-135 | 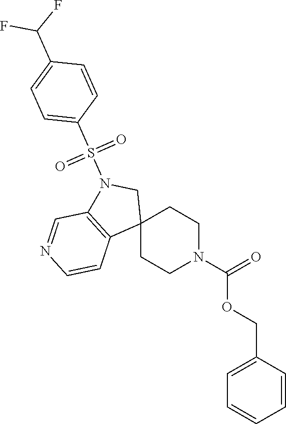 [SMILES: O=C(OCc1ccccc1)N1CCC2(CC1)CN(S(=O)(=O)c1ccc(C(F)F)cc1)c1cnccc12] |
| I-136 | 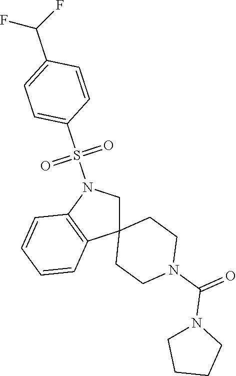 [SMILES: O=C(N1CCCC1)N1CCC2(CC1)CN(S(=O)(=O)c1ccc(C(F)F)cc1)c1ccccc12] |
| I-137 | 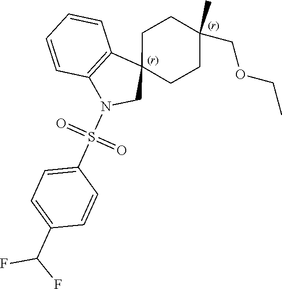 [SMILES: CCOC[C@]1(C)CC[C@]2(CC1)CN(S(=O)(=O)c1ccc(C(F)F)cc1)c1ccccc12] |
| I-138 | 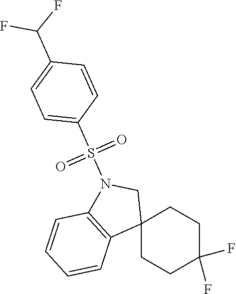 [SMILES: O=S(=O)(c1ccc(C(F)F)cc1)N1CC2(CCC(F)(F)CC2)c2ccccc21] |
| I-139 | 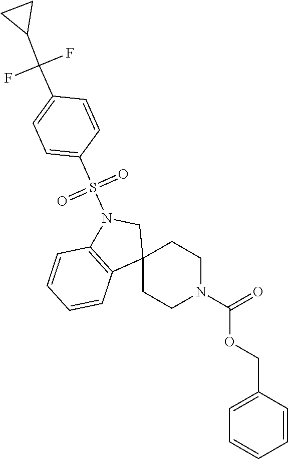 [SMILES: O=C(OCc1ccccc1)N1CCC2(CC1)CN(S(=O)(=O)c1ccc(C(F)(F)C3CC3)cc1)c1ccccc12] |
| I-140 | 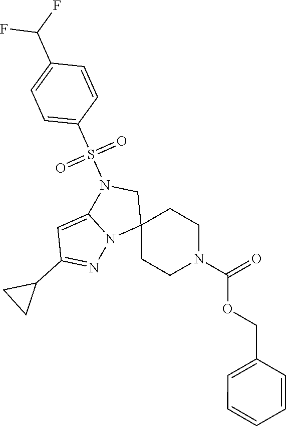 [SMILES: O=C(OCc1ccccc1)N1CCC2(CC1)CN(S(=O)(=O)c1ccc(C(F)F)cc1)c1cc(C3CC3)nn12] |
| I-141 | 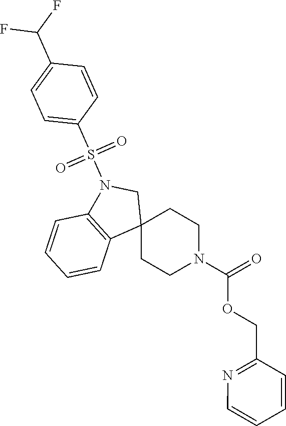 [SMILES: O=C(OCc1ccccn1)N1CCC2(CC1)CN(S(=O)(=O)c1ccc(C(F)F)cc1)c1ccccc12] |
| I-142 | 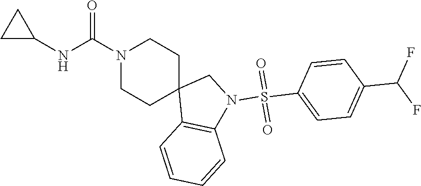 [SMILES: O=C(NC1CC1)N1CCC2(CC1)CN(S(=O)(=O)c1ccc(C(F)F)cc1)c1ccccc12] |
| I-143 | 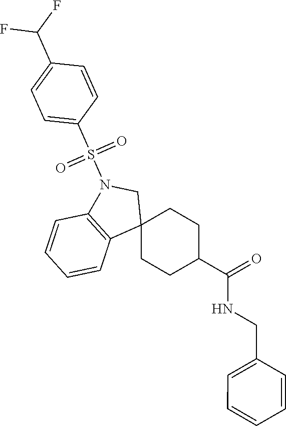 [SMILES: O=C(NCc1ccccc1)C1CCC2(CC1)CN(S(=O)(=O)c1ccc(C(F)F)cc1)c1ccccc12] |
| I-144 | 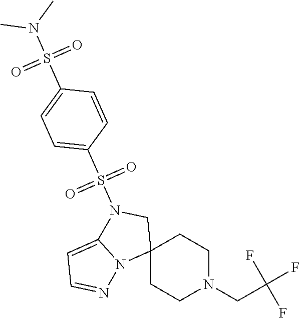 [SMILES: CN(C)S(=O)(=O)c1ccc(S(=O)(=O)N2CC3(CCN(CC(F)(F)F)CC3)n3nccc32)cc1] |
| I-145 | 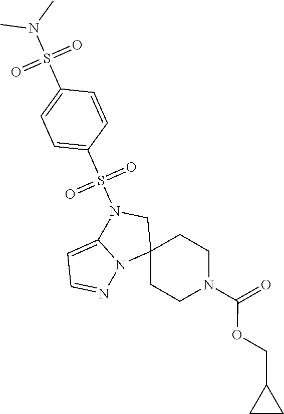 [SMILES: CN(C)S(=O)(=O)c1ccc(S(=O)(=O)N2CC3(CCN(C(=O)OCC4CC4)CC3)n3nccc32)cc1] |
| I-146 | 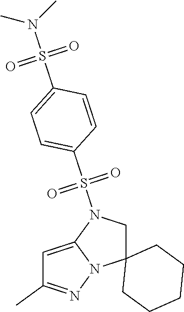 [SMILES: Cc1cc2n(n1)C1(CCCCC1)CN2S(=O)(=O)c1ccc(S(=O)(=O)N(C)C)cc1] |
| I-147 | 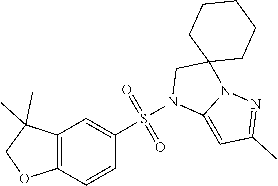 |
| I-148 | 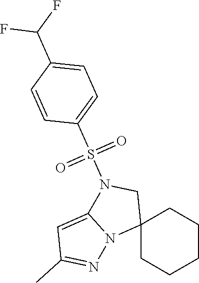 [SMILES: Cc1cc2n(n1)C1(CCCCC1)CN2S(=O)(=O)c1ccc(C(F)F)cc1] |
| I-149 | 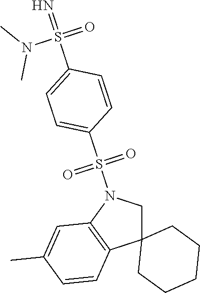 [SMILES: Cc1ccc2c(c1)N(S(=O)(=O)c1ccc(S(=N)(=O)N(C)C)cc1)CC21CCCCC1] |
| I-150 | 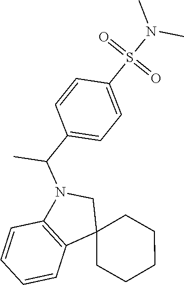 [SMILES: CC(c1ccc(S(=O)(=O)N(C)C)cc1)N1CC2(CCCCC2)c2ccccc21] |
| I-151 | 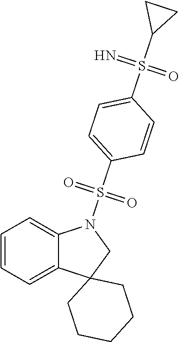 [SMILES: N=S(=O)(c1ccc(S(=O)(=O)N2CC3(CCCCC3)c3ccccc32)cc1)C1CC1] |
| I-152 | 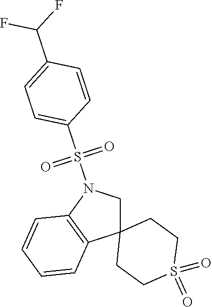 [SMILES: O=S1(=O)CCC2(CC1)CN(S(=O)(=O)c1ccc(C(F)F)cc1)c1ccccc12] |
| I-153 | 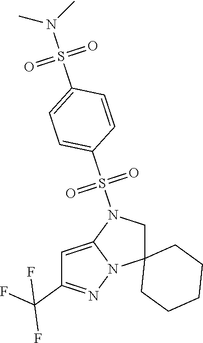 [SMILES: CN(C)S(=O)(=O)c1ccc(S(=O)(=O)N2CC3(CCCCC3)n3nc(C(F)(F)F)cc32)cc1] |
| I-154 | 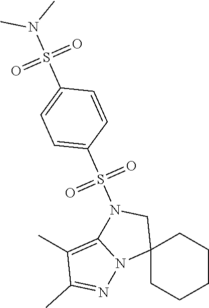 [SMILES: Cc1nn2c(c1C)N(S(=O)(=O)c1ccc(S(=O)(=O)N(C)C)cc1)CC21CCCCC1] |
| I-155 | 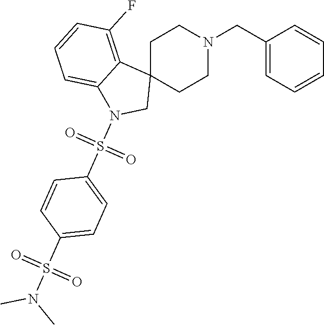 [SMILES: CN(C)S(=O)(=O)c1ccc(S(=O)(=O)N2CC3(CCN(Cc4ccccc4)CC3)c3c(F)cccc32)cc1] |
| I-157 | 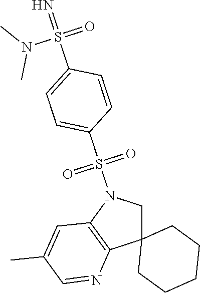 [SMILES: Cc1cnc2c(c1)N(S(=O)(=O)c1ccc(S(=N)(=O)N(C)C)cc1)CC21CCCCC1] |
| I-158 | 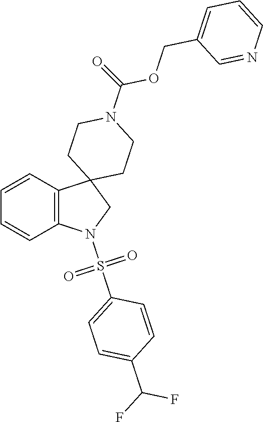 [SMILES: O=C(OCc1cccnc1)N1CCC2(CC1)CN(S(=O)(=O)c1ccc(C(F)F)cc1)c1ccccc12] |
| I-159 | 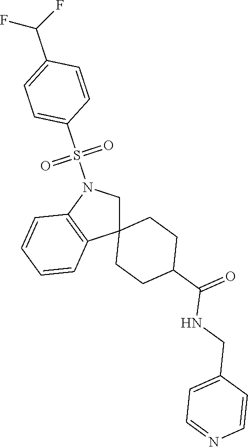 [SMILES: O=C(NCc1ccncc1)C1CCC2(CC1)CN(S(=O)(=O)c1ccc(C(F)F)cc1)c1ccccc12] |
| I-160 | 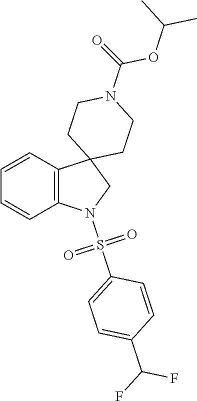 [SMILES: CC(C)OC(=O)N1CCC2(CC1)CN(S(=O)(=O)c1ccc(C(F)F)cc1)c1ccccc12] |
| I-161 | 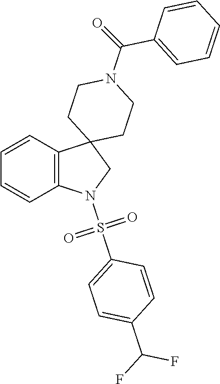 [SMILES: O=C(c1ccccc1)N1CCC2(CC1)CN(S(=O)(=O)c1ccc(C(F)F)cc1)c1ccccc12] |
| I-162 | 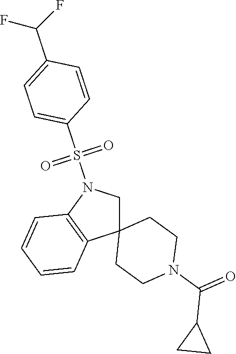 [SMILES: O=C(C1CC1)N1CCC2(CC1)CN(S(=O)(=O)c1ccc(C(F)F)cc1)c1ccccc12] |
| I-163 | 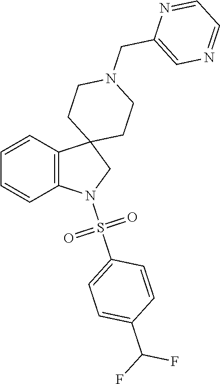 [SMILES: O=S(=O)(c1ccc(C(F)F)cc1)N1CC2(CCN(Cc3cnccn3)CC2)c2ccccc21] |
| I-164 | 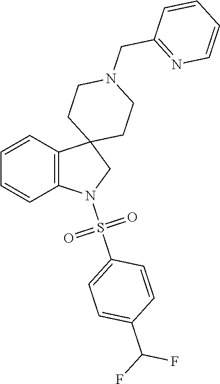 [SMILES: O=S(=O)(c1ccc(C(F)F)cc1)N1CC2(CCN(Cc3ccccn3)CC2)c2ccccc21] |
| I-165 | 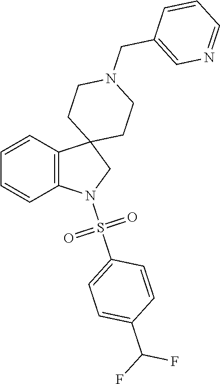 [SMILES: O=S(=O)(c1ccc(C(F)F)cc1)N1CC2(CCN(Cc3cccnc3)CC2)c2ccccc21] |
| I-166 | 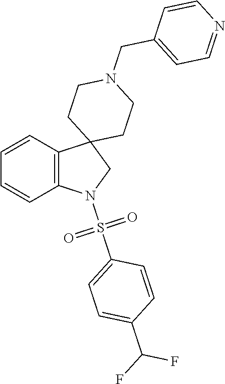 [SMILES: O=S(=O)(c1ccc(C(F)F)cc1)N1CC2(CCN(Cc3ccncc3)CC2)c2ccccc21] |
| I-167 | 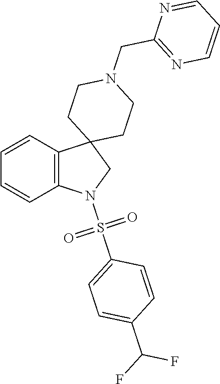 [SMILES: O=S(=O)(c1ccc(C(F)F)cc1)N1CC2(CCN(Cc3ncccn3)CC2)c2ccccc21] |
| I-168 | 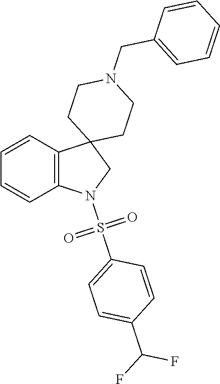 [SMILES: O=S(=O)(c1ccc(C(F)F)cc1)N1CC2(CCN(Cc3ccccc3)CC2)c2ccccc21] |
| I-169 | 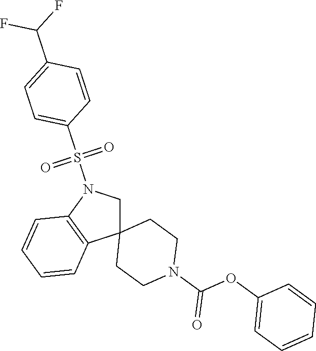 [SMILES: O=C(Oc1ccccc1)N1CCC2(CC1)CN(S(=O)(=O)c1ccc(C(F)F)cc1)c1ccccc12] |
| I-170 | 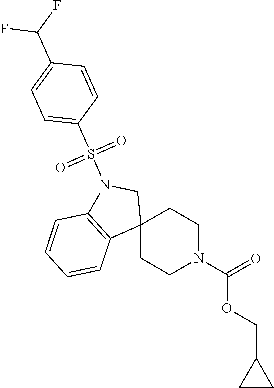 [SMILES: O=C(OCC1CC1)N1CCC2(CC1)CN(S(=O)(=O)c1ccc(C(F)F)cc1)c1ccccc12] |
| I-171 | 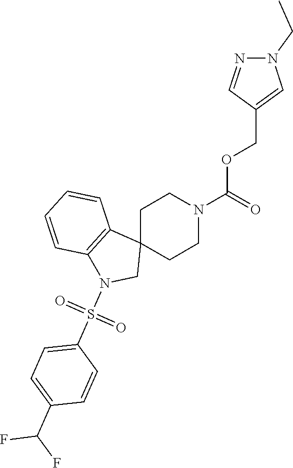 [SMILES: CCn1cc(COC(=O)N2CCC3(CC2)CN(S(=O)(=O)c2ccc(C(F)F)cc2)c2ccccc23)cn1] |
| I-172 | 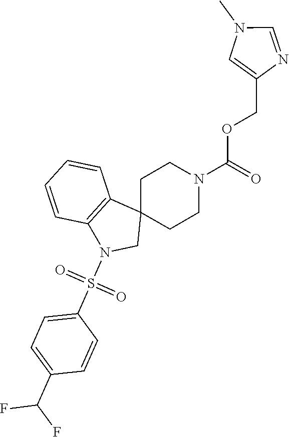 [SMILES: Cn1cnc(COC(=O)N2CCC3(CC2)CN(S(=O)(=O)c2ccc(C(F)F)cc2)c2ccccc23)c1] |
| I-173 | 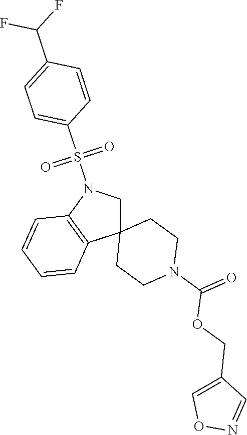 [SMILES: O=C(OCc1cnoc1)N1CCC2(CC1)CN(S(=O)(=O)c1ccc(C(F)F)cc1)c1ccccc12] |
| I-174 | 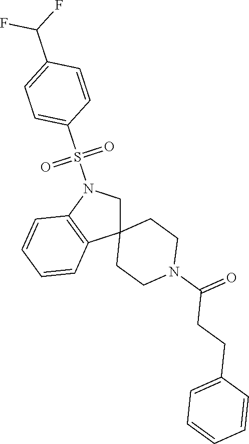 [SMILES: O=C(CCc1ccccc1)N1CCC2(CC1)CN(S(=O)(=O)c1ccc(C(F)F)cc1)c1ccccc12] |
| I-175 | 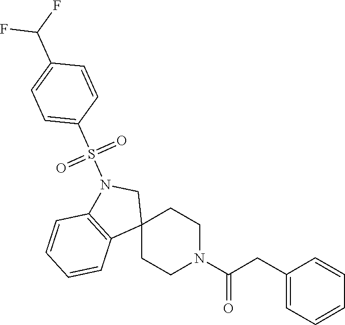 [SMILES: O=C(Cc1ccccc1)N1CCC2(CC1)CN(S(=O)(=O)c1ccc(C(F)F)cc1)c1ccccc12] |
| I-176 | 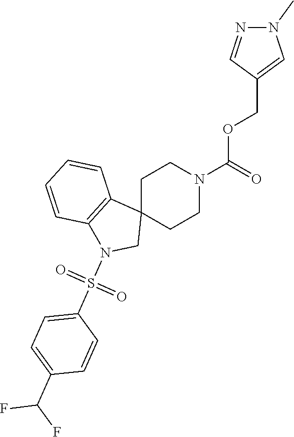 [SMILES: Cn1cc(COC(=O)N2CCC3(CC2)CN(S(=O)(=O)c2ccc(C(F)F)cc2)c2ccccc23)cn1] |
| I-177 | 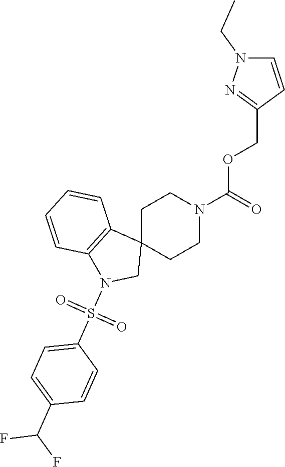 [SMILES: CCn1ccc(COC(=O)N2CCC3(CC2)CN(S(=O)(=O)c2ccc(C(F)F)cc2)c2ccccc23)n1] |
| I-178 | 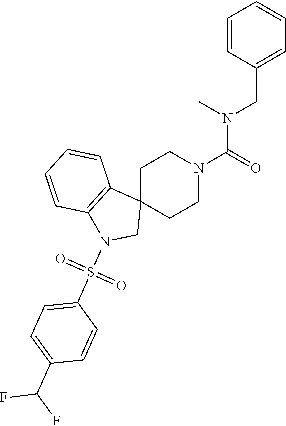 [SMILES: CN(Cc1ccccc1)C(=O)N1CCC2(CC1)CN(S(=O)(=O)c1ccc(C(F)F)cc1)c1ccccc12] |
| I-179 | 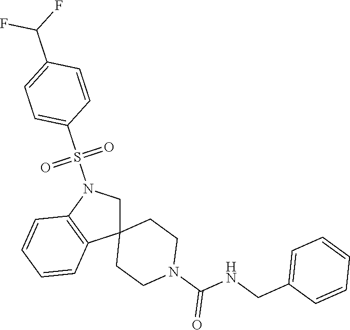 [SMILES: O=C(NCc1ccccc1)N1CCC2(CC1)CN(S(=O)(=O)c1ccc(C(F)F)cc1)c1ccccc12] |
| I-180 | 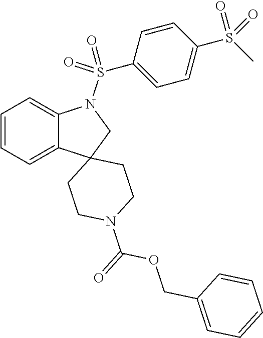 [SMILES: CS(=O)(=O)c1ccc(S(=O)(=O)N2CC3(CCN(C(=O)OCc4ccccc4)CC3)c3ccccc32)cc1] |
| I-181 | 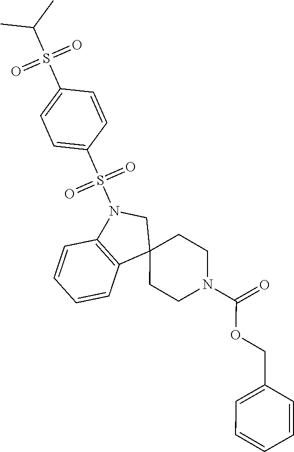 [SMILES: CC(C)S(=O)(=O)c1ccc(S(=O)(=O)N2CC3(CCN(C(=O)OCc4ccccc4)CC3)c3ccccc32)cc1] |
| I-182 | 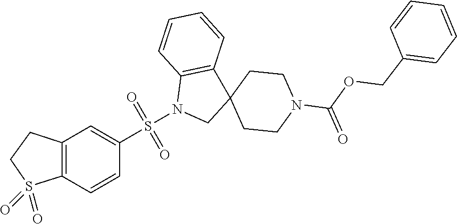 [SMILES: O=C(OCc1ccccc1)N1CCC2(CC1)CN(S(=O)(=O)c1ccc3c(c1)CCS3(=O)=O)c1ccccc12] |
| I-183 | 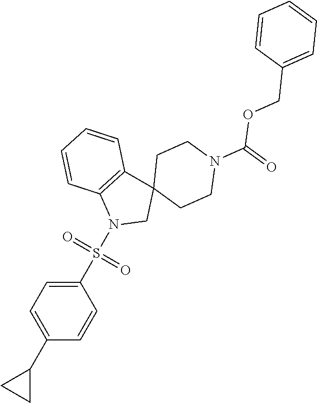 [SMILES: O=C(OCc1ccccc1)N1CCC2(CC1)CN(S(=O)(=O)c1ccc(C3CC3)cc1)c1ccccc12] |
| I-184 | 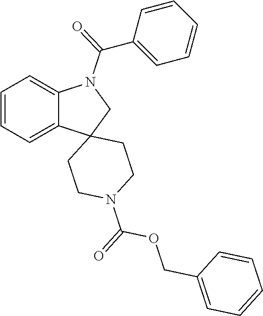 [SMILES: O=C(OCc1ccccc1)N1CCC2(CC1)CN(C(=O)c1ccccc1)c1ccccc12] |
| I-185 | 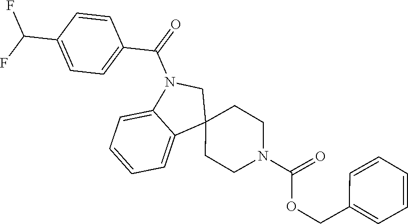 [SMILES: O=C(OCc1ccccc1)N1CCC2(CC1)CN(C(=O)c1ccc(C(F)F)cc1)c1ccccc12] |
| I-186 | 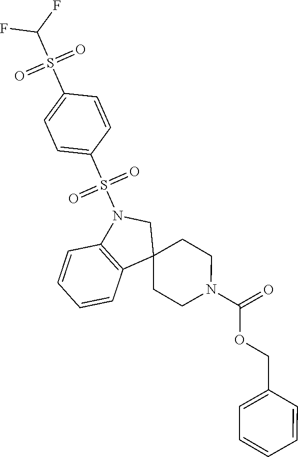 [SMILES: O=C(OCc1ccccc1)N1CCC2(CC1)CN(S(=O)(=O)c1ccc(S(=O)(=O)C(F)F)cc1)c1ccccc12] |
| I-187 | 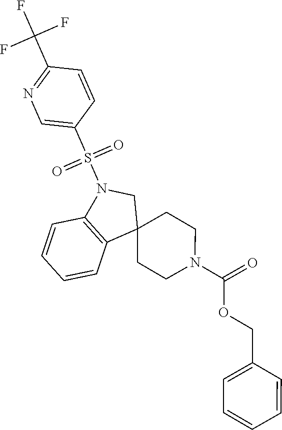 [SMILES: O=C(OCc1ccccc1)N1CCC2(CC1)CN(S(=O)(=O)c1ccc(C(F)(F)F)nc1)c1ccccc12] |
| I-188 | 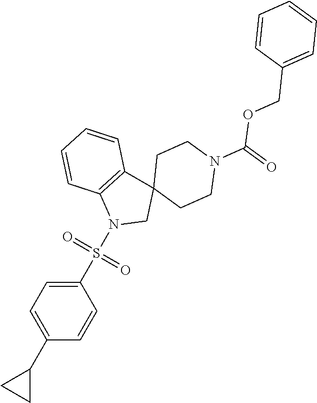 [SMILES: O=C(OCc1ccccc1)N1CCC2(CC1)CN(S(=O)(=O)c1ccc(C3CC3)cc1)c1ccccc12] |
| I-189 | 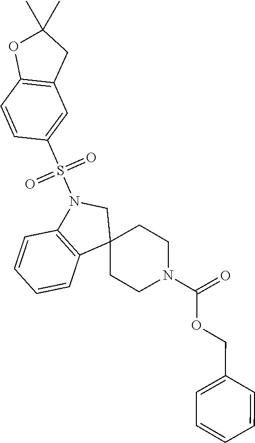 [SMILES: CC1(C)Cc2cc(S(=O)(=O)N3CC4(CCN(C(=O)OCc5ccccc5)CC4)c4ccccc43)ccc2O1] |
| I-190 | 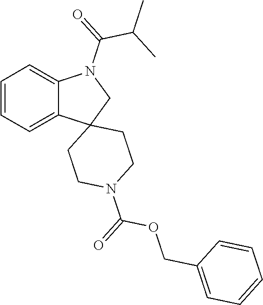 [SMILES: CC(C)C(=O)N1CC2(CCN(C(=O)OCc3ccccc3)CC2)c2ccccc21] |
| I-191 | 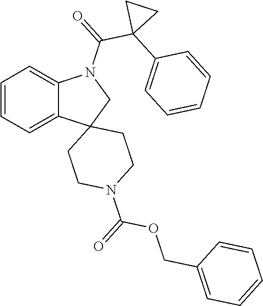 [SMILES: O=C(OCc1ccccc1)N1CCC2(CC1)CN(C(=O)C1(c3ccccc3)CC1)c1ccccc12] |
| I-192 | 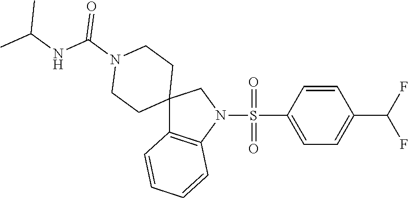 [SMILES: CC(C)NC(=O)N1CCC2(CC1)CN(S(=O)(=O)c1ccc(C(F)F)cc1)c1ccccc12] |
| I-193 | 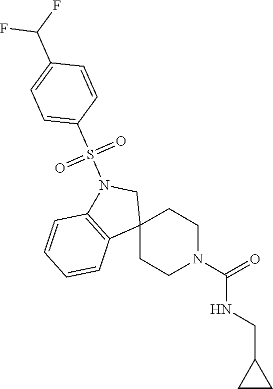 [SMILES: O=C(NCC1CC1)N1CCC2(CC1)CN(S(=O)(=O)c1ccc(C(F)F)cc1)c1ccccc12] |
| I-194 | 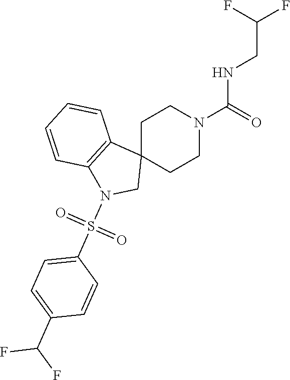 [SMILES: O=C(NCC(F)F)N1CCC2(CC1)CN(S(=O)(=O)c1ccc(C(F)F)cc1)c1ccccc12] |
| I-195 | 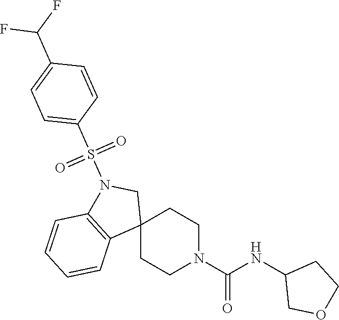 [SMILES: O=C(NC1CCOC1)N1CCC2(CC1)CN(S(=O)(=O)c1ccc(C(F)F)cc1)c1ccccc12] |
| I-196 | 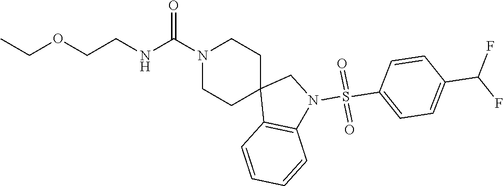 [SMILES: CCOCCNC(=O)N1CCC2(CC1)CN(S(=O)(=O)c1ccc(C(F)F)cc1)c1ccccc12] |
| I-197 | 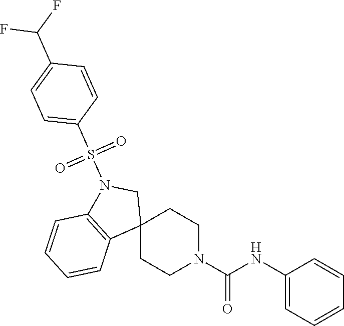 [SMILES: O=C(Nc1ccccc1)N1CCC2(CC1)CN(S(=O)(=O)c1ccc(C(F)F)cc1)c1ccccc12] |
| I-198 | 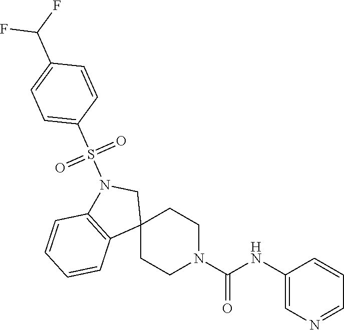 [SMILES: O=C(Nc1cccnc1)N1CCC2(CC1)CN(S(=O)(=O)c1ccc(C(F)F)cc1)c1ccccc12] |
| I-199 | 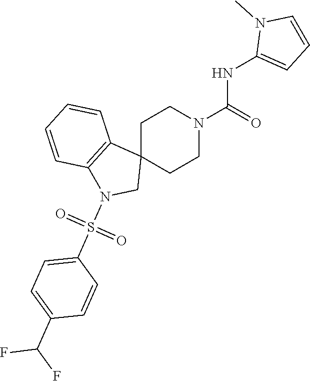 [SMILES: Cn1cccc1NC(=O)N1CCC2(CC1)CN(S(=O)(=O)c1ccc(C(F)F)cc1)c1ccccc12] |
| I-200 | 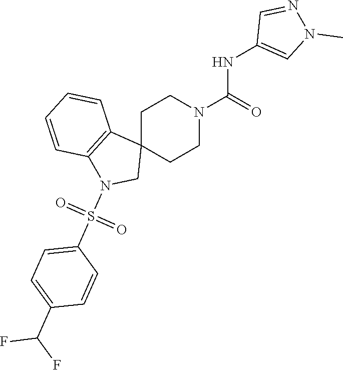 [SMILES: Cn1cc(NC(=O)N2CCC3(CC2)CN(S(=O)(=O)c2ccc(C(F)F)cc2)c2ccccc23)cn1] |
| I-201 | 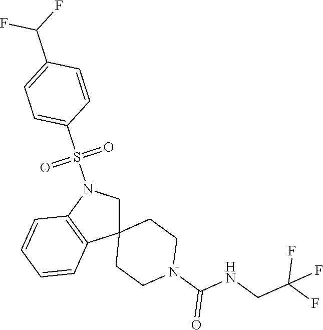 [SMILES: O=C(NCC(F)(F)F)N1CCC2(CC1)CN(S(=O)(=O)c1ccc(C(F)F)cc1)c1ccccc12] |
| I-202 | 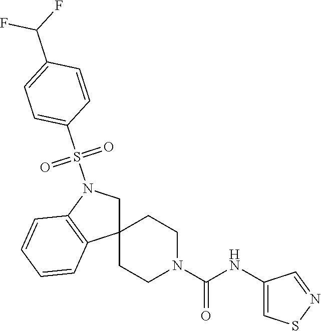 [SMILES: O=C(Nc1cnsc1)N1CCC2(CC1)CN(S(=O)(=O)c1ccc(C(F)F)cc1)c1ccccc12] |
| I-203 | 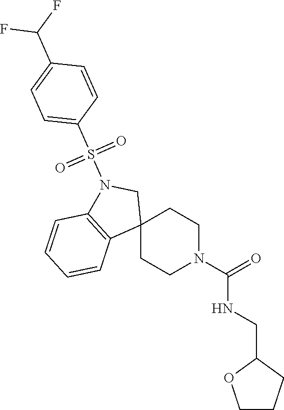 [SMILES: O=C(NCC1CCCO1)N1CCC2(CC1)CN(S(=O)(=O)c1ccc(C(F)F)cc1)c1ccccc12] |
| I-204 | 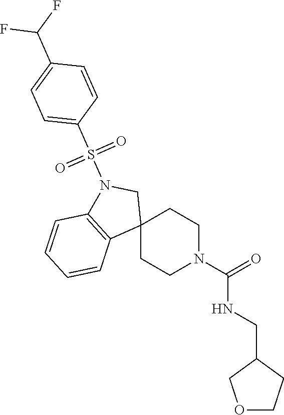 [SMILES: O=C(NCC1CCOC1)N1CCC2(CC1)CN(S(=O)(=O)c1ccc(C(F)F)cc1)c1ccccc12] |
| I-205 | 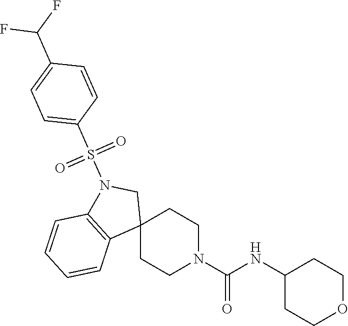 [SMILES: O=C(NC1CCOCC1)N1CCC2(CC1)CN(S(=O)(=O)c1ccc(C(F)F)cc1)c1ccccc12] |
| I-206 | 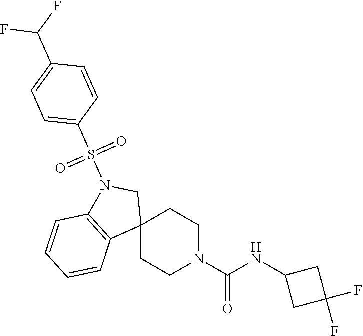 [SMILES: O=C(NC1CC(F)(F)C1)N1CCC2(CC1)CN(S(=O)(=O)c1ccc(C(F)F)cc1)c1ccccc12] |
| I-207 | 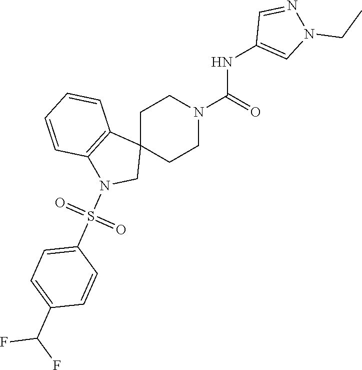 [SMILES: CCn1cc(NC(=O)N2CCC3(CC2)CN(S(=O)(=O)c2ccc(C(F)F)cc2)c2ccccc23)cn1] |
| I-208 | 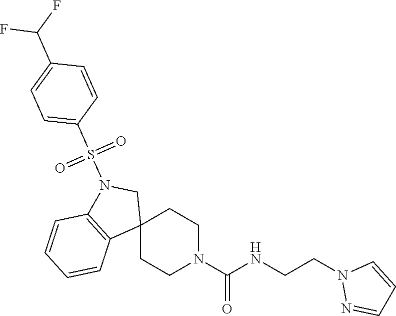 [SMILES: O=C(NCCn1cccn1)N1CCC2(CC1)CN(S(=O)(=O)c1ccc(C(F)F)cc1)c1ccccc12] |
| I-209 | 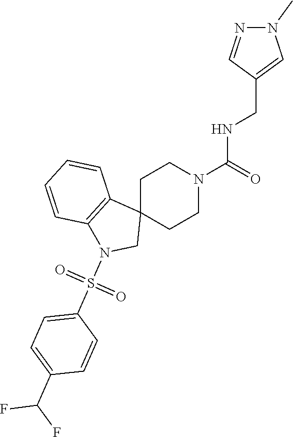 [SMILES: Cn1cc(CNC(=O)N2CCC3(CC2)CN(S(=O)(=O)c2ccc(C(F)F)cc2)c2ccccc23)cn1] |
| I-210 | 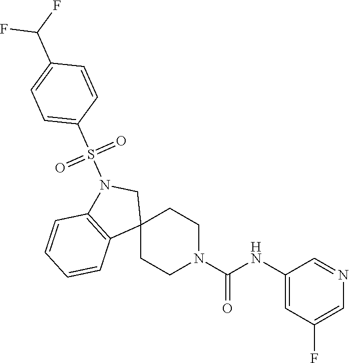 [SMILES: O=C(Nc1cncc(F)c1)N1CCC2(CC1)CN(S(=O)(=O)c1ccc(C(F)F)cc1)c1ccccc12] |
| I-211 | 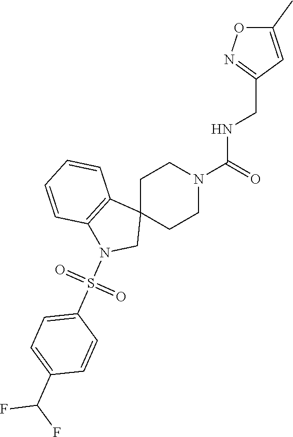 [SMILES: Cc1cc(CNC(=O)N2CCC3(CC2)CN(S(=O)(=O)c2ccc(C(F)F)cc2)c2ccccc23)no1] |
| I-212 | 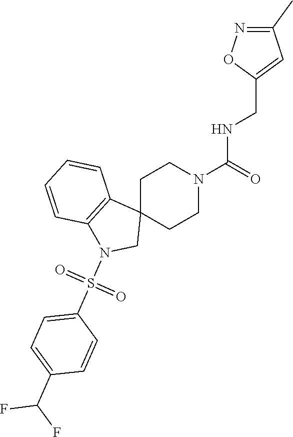 [SMILES: Cc1cc(CNC(=O)N2CCC3(CC2)CN(S(=O)(=O)c2ccc(C(F)F)cc2)c2ccccc23)on1] |
| I-213 | 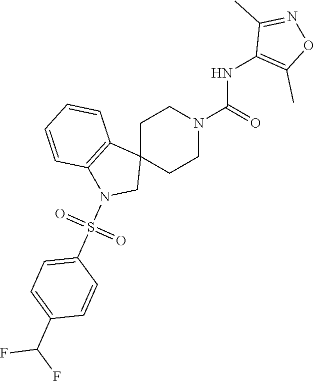 [SMILES: Cc1noc(C)c1NC(=O)N1CCC2(CC1)CN(S(=O)(=O)c1ccc(C(F)F)cc1)c1ccccc12] |
| I-214 | 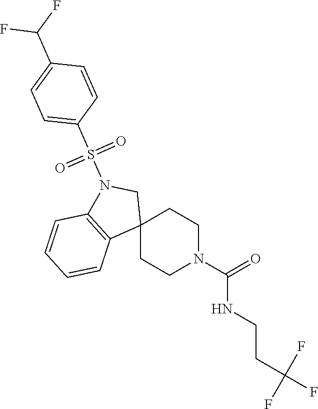 [SMILES: O=C(NCCC(F)(F)F)N1CCC2(CC1)CN(S(=O)(=O)c1ccc(C(F)F)cc1)c1ccccc12] |
| I-215 | 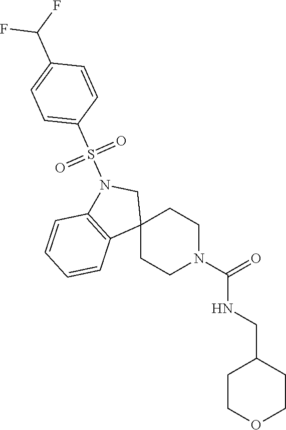 [SMILES: O=C(NCC1CCOCC1)N1CCC2(CC1)CN(S(=O)(=O)c1ccc(C(F)F)cc1)c1ccccc12] |
| I-216 | 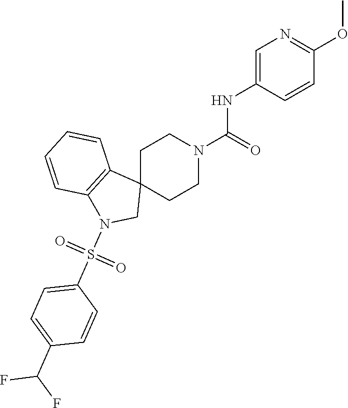 [SMILES: COc1ccc(NC(=O)N2CCC3(CC2)CN(S(=O)(=O)c2ccc(C(F)F)cc2)c2ccccc23)cn1] |
| I-217 | 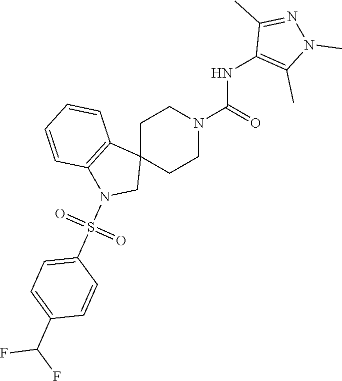 [SMILES: Cc1nn(C)c(C)c1NC(=O)N1CCC2(CC1)CN(S(=O)(=O)c1ccc(C(F)F)cc1)c1ccccc12] |
| I-218 | 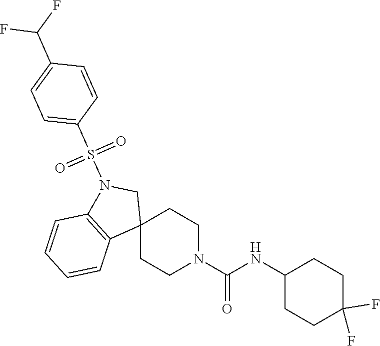 [SMILES: O=C(NC1CCC(F)(F)CC1)N1CCC2(CC1)CN(S(=O)(=O)c1ccc(C(F)F)cc1)c1ccccc12] |
| I-219 | 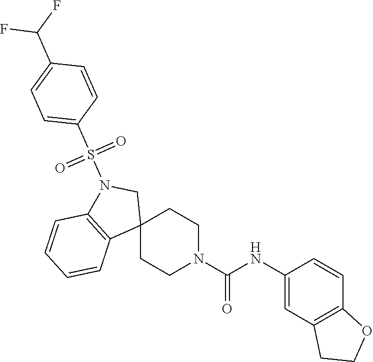 [SMILES: O=C(Nc1ccc2c(c1)CCO2)N1CCC2(CC1)CN(S(=O)(=O)c1ccc(C(F)F)cc1)c1ccccc12] |
| I-220 | 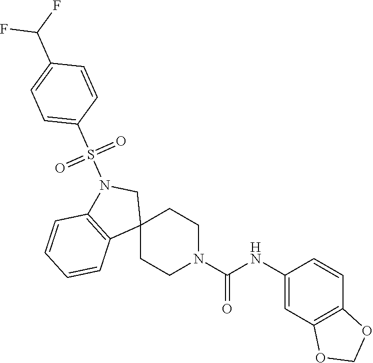 [SMILES: O=C(Nc1ccc2c(c1)OCO2)N1CCC2(CC1)CN(S(=O)(=O)c1ccc(C(F)F)cc1)c1ccccc12] |
| I-221 | 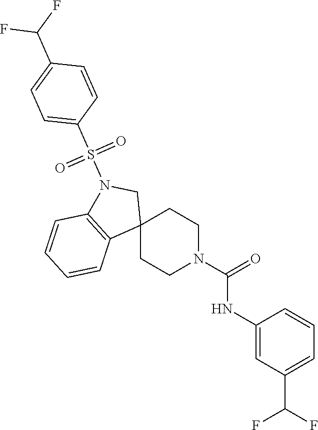 [SMILES: O=C(Nc1cccc(C(F)F)c1)N1CCC2(CC1)CN(S(=O)(=O)c1ccc(C(F)F)cc1)c1ccccc12] |
| I-222 | 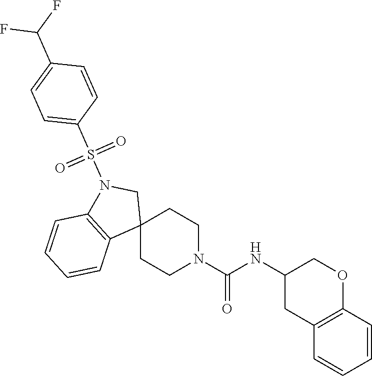 [SMILES: O=C(NC1COc2ccccc2C1)N1CCC2(CC1)CN(S(=O)(=O)c1ccc(C(F)F)cc1)c1ccccc12] |
| I-223 | 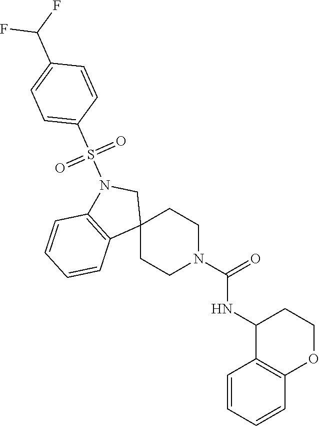 [SMILES: O=C(NC1CCOc2ccccc21)N1CCC2(CC1)CN(S(=O)(=O)c1ccc(C(F)F)cc1)c1ccccc12] |
| I-224 | 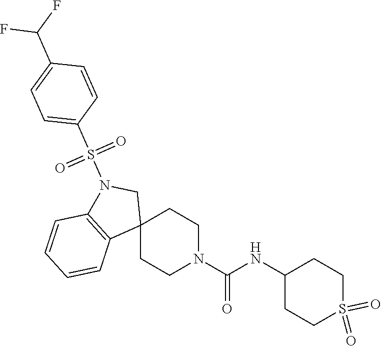 [SMILES: O=C(NC1CCS(=O)(=O)CC1)N1CCC2(CC1)CN(S(=O)(=O)c1ccc(C(F)F)cc1)c1ccccc12] |
| I-225 | 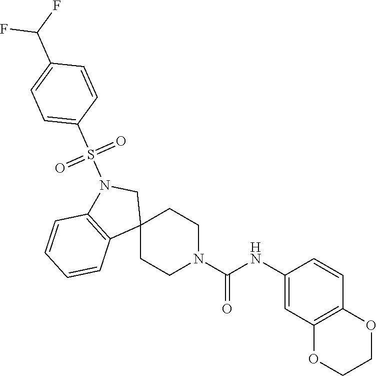 [SMILES: O=C(Nc1ccc2c(c1)OCCO2)N1CCC2(CC1)CN(S(=O)(=O)c1ccc(C(F)F)cc1)c1ccccc12] |
| I-226 | 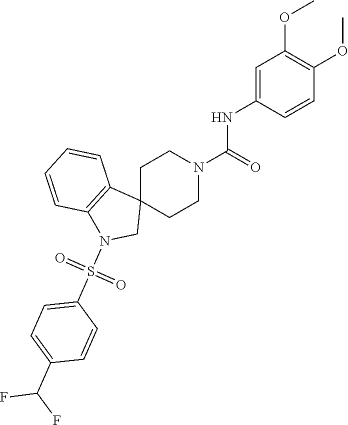 [SMILES: COc1ccc(NC(=O)N2CCC3(CC2)CN(S(=O)(=O)c2ccc(C(F)F)cc2)c2ccccc23)cc1OC] |
| I-227 | 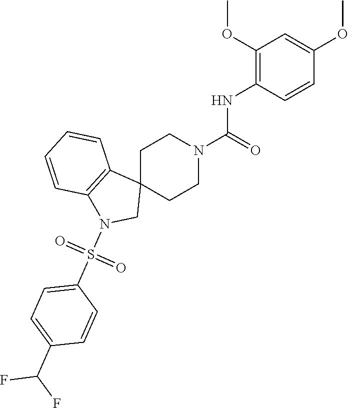 [SMILES: COc1ccc(NC(=O)N2CCC3(CC2)CN(S(=O)(=O)c2ccc(C(F)F)cc2)c2ccccc23)c(OC)c1] |
| I-228 | 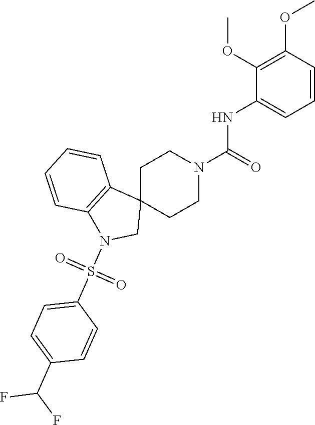 [SMILES: COc1cccc(NC(=O)N2CCC3(CC2)CN(S(=O)(=O)c2ccc(C(F)F)cc2)c2ccccc23)c1OC] |
| I-229 | 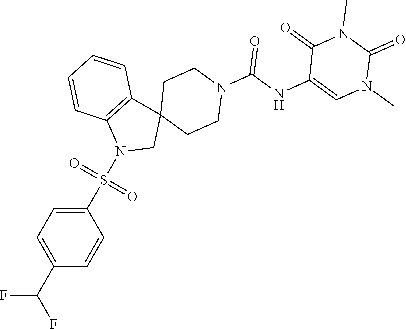 [SMILES: Cn1cc(NC(=O)N2CCC3(CC2)CN(S(=O)(=O)c2ccc(C(F)F)cc2)c2ccccc23)c(=O)n(C)c1=O] |
| I-230 | 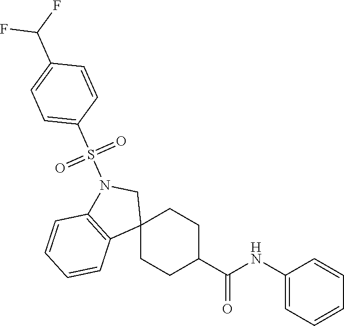 [SMILES: O=C(Nc1ccccc1)C1CCC2(CC1)CN(S(=O)(=O)c1ccc(C(F)F)cc1)c1ccccc12] |
| I-231 | 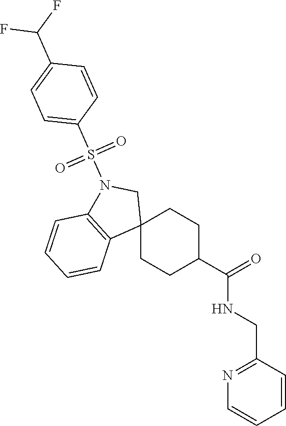 [SMILES: O=C(NCc1ccccn1)C1CCC2(CC1)CN(S(=O)(=O)c1ccc(C(F)F)cc1)c1ccccc12] |
| I-232 | 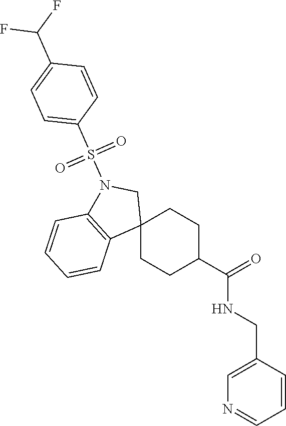 [SMILES: O=C(NCc1cccnc1)C1CCC2(CC1)CN(S(=O)(=O)c1ccc(C(F)F)cc1)c1ccccc12] |
| I-233 | 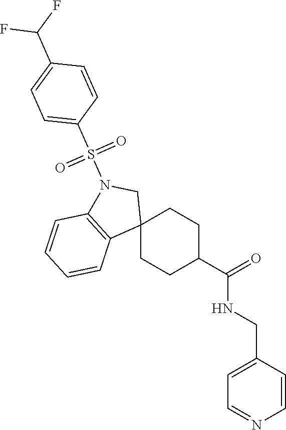 [SMILES: O=C(NCc1ccncc1)C1CCC2(CC1)CN(S(=O)(=O)c1ccc(C(F)F)cc1)c1ccccc12] |
| I-234 | 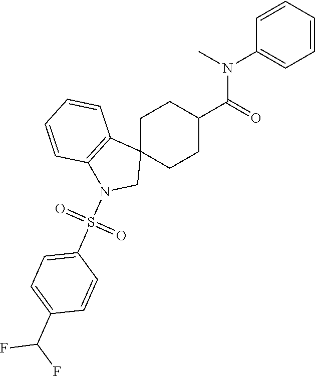 [SMILES: CN(C(=O)C1CCC2(CC1)CN(S(=O)(=O)c1ccc(C(F)F)cc1)c1ccccc12)c1ccccc1] |
| I-235 | 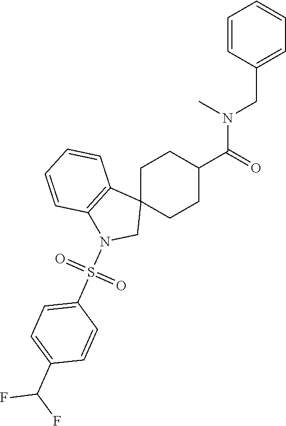 [SMILES: CN(Cc1ccccc1)C(=O)C1CCC2(CC1)CN(S(=O)(=O)c1ccc(C(F)F)cc1)c1ccccc12] |
| I-236 | 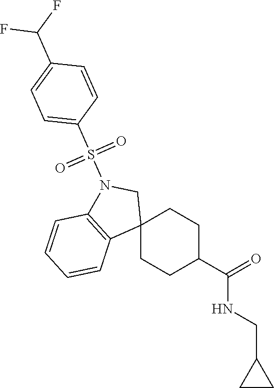 [SMILES: O=C(NCC1CC1)C1CCC2(CC1)CN(S(=O)(=O)c1ccc(C(F)F)cc1)c1ccccc12] |
| I-237 | 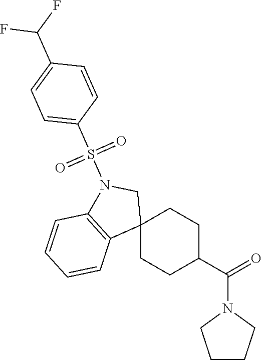 [SMILES: O=C(C1CCC2(CC1)CN(S(=O)(=O)c1ccc(C(F)F)cc1)c1ccccc12)N1CCCC1] |
| I-238 | 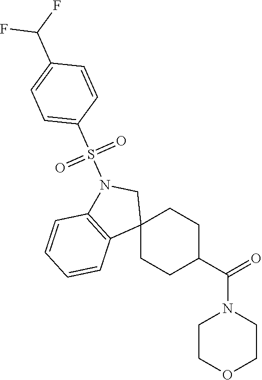 [SMILES: O=C(C1CCC2(CC1)CN(S(=O)(=O)c1ccc(C(F)F)cc1)c1ccccc12)N1CCOCC1] |
| I-239 | 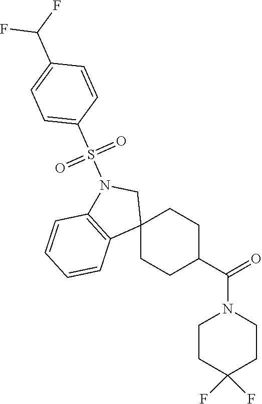 [SMILES: O=C(C1CCC2(CC1)CN(S(=O)(=O)c1ccc(C(F)F)cc1)c1ccccc12)N1CCC(F)(F)CC1] |
| I-240 | 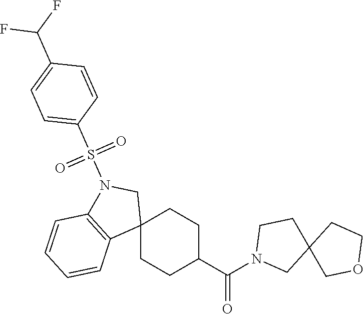 [SMILES: O=C(C1CCC2(CC1)CN(S(=O)(=O)c1ccc(C(F)F)cc1)c1ccccc12)N1CCC2(CCOC2)C1] |
| I-241 | 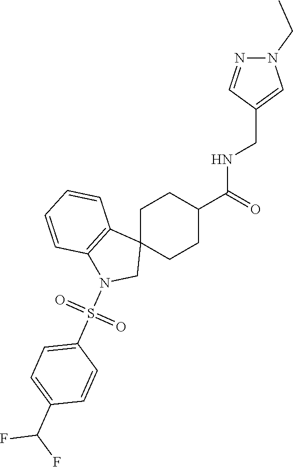 [SMILES: CCn1cc(CNC(=O)C2CCC3(CC2)CN(S(=O)(=O)c2ccc(C(F)F)cc2)c2ccccc23)cn1] |
| I-242 | 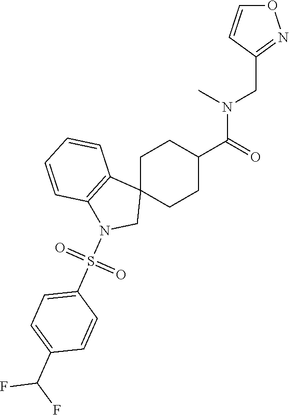 [SMILES: CN(Cc1ccon1)C(=O)C1CCC2(CC1)CN(S(=O)(=O)c1ccc(C(F)F)cc1)c1ccccc12] |
| I-243 | 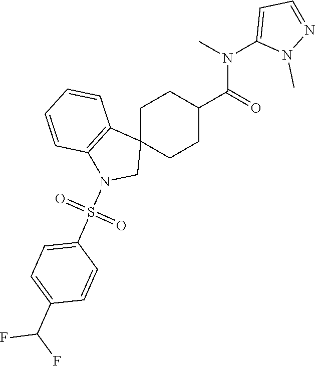 [SMILES: CN(C(=O)C1CCC2(CC1)CN(S(=O)(=O)c1ccc(C(F)F)cc1)c1ccccc12)c1ccnn1C] |
| I-244 | 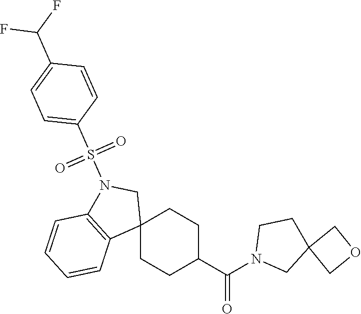 [SMILES: O=C(C1CCC2(CC1)CN(S(=O)(=O)c1ccc(C(F)F)cc1)c1ccccc12)N1CCC2(COC2)C1] |
| I-245 | 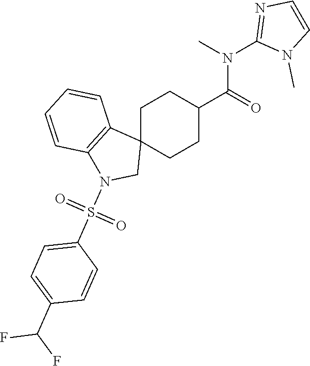 [SMILES: CN(C(=O)C1CCC2(CC1)CN(S(=O)(=O)c1ccc(C(F)F)cc1)c1ccccc12)c1nccn1C] |
| I-246 | 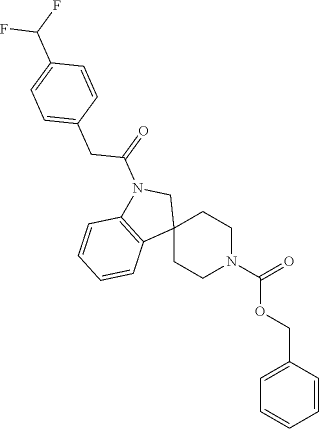 [SMILES: O=C(OCc1ccccc1)N1CCC2(CC1)CN(C(=O)Cc1ccc(C(F)F)cc1)c1ccccc12] |
| I-247 | 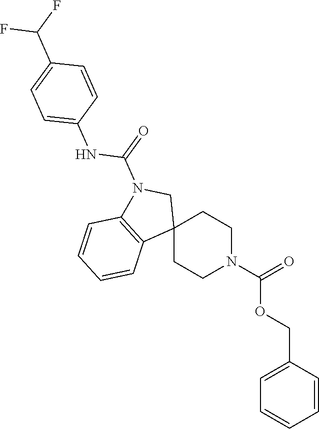 [SMILES: O=C(OCc1ccccc1)N1CCC2(CC1)CN(C(=O)Nc1ccc(C(F)F)cc1)c1ccccc12] |
| I-248 | 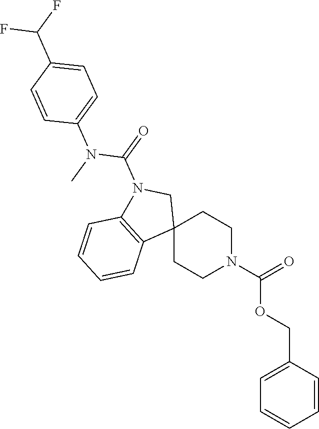 [SMILES: CN(C(=O)N1CC2(CCN(C(=O)OCc3ccccc3)CC2)c2ccccc21)c1ccc(C(F)F)cc1] |
| I-249 | 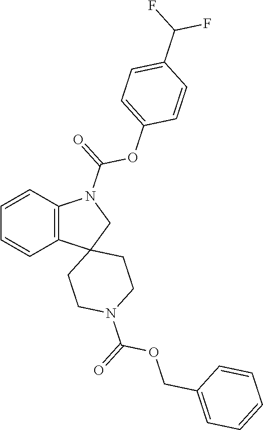 [SMILES: O=C(OCc1ccccc1)N1CCC2(CC1)CN(C(=O)Oc1ccc(C(F)F)cc1)c1ccccc12] |
| I-250 | 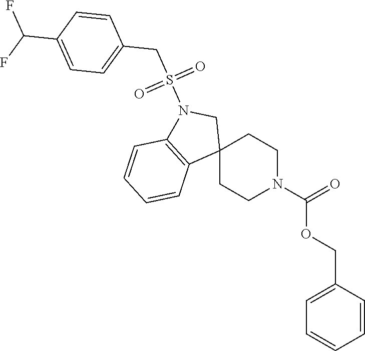 [SMILES: O=C(OCc1ccccc1)N1CCC2(CC1)CN(S(=O)(=O)Cc1ccc(C(F)F)cc1)c1ccccc12] |
| I-251 | 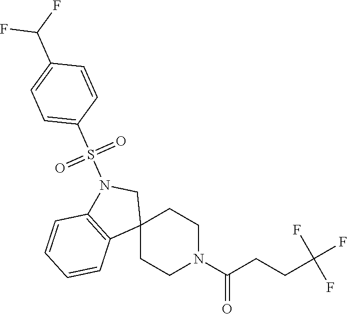 [SMILES: O=C(CCC(F)(F)F)N1CCC2(CC1)CN(S(=O)(=O)c1ccc(C(F)F)cc1)c1ccccc12] |
| I-252 | 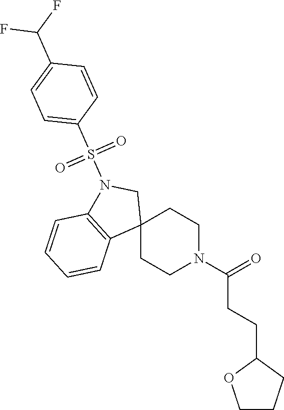 [SMILES: O=C(CCC1CCCO1)N1CCC2(CC1)CN(S(=O)(=O)c1ccc(C(F)F)cc1)c1ccccc12] |
| I-253 | 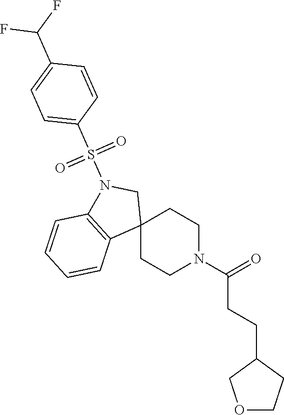 [SMILES: O=C(CCC1CCOC1)N1CCC2(CC1)CN(S(=O)(=O)c1ccc(C(F)F)cc1)c1ccccc12] |
| I-254 | 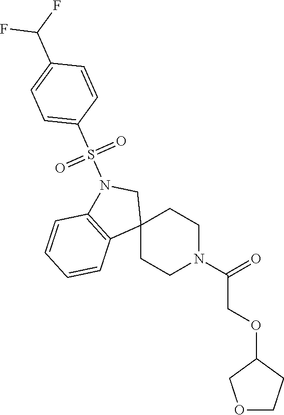 [SMILES: O=C(COC1CCOC1)N1CCC2(CC1)CN(S(=O)(=O)c1ccc(C(F)F)cc1)c1ccccc12] |
| I-255 | 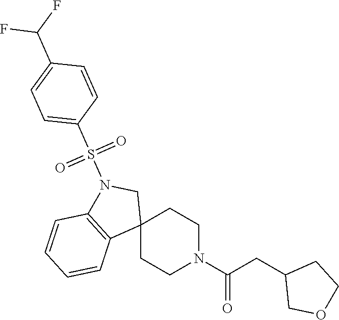 [SMILES: O=C(CC1CCOC1)N1CCC2(CC1)CN(S(=O)(=O)c1ccc(C(F)F)cc1)c1ccccc12] |
| I-256 | 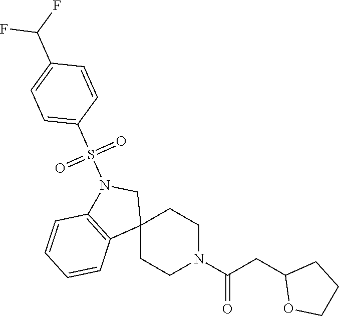 [SMILES: O=C(CC1CCCO1)N1CCC2(CC1)CN(S(=O)(=O)c1ccc(C(F)F)cc1)c1ccccc12] |
| I-257 | 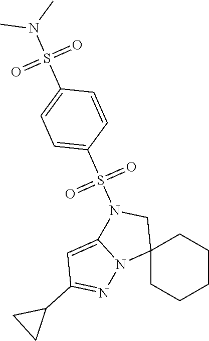 [SMILES: CN(C)S(=O)(=O)c1ccc(S(=O)(=O)N2CC3(CCCCC3)n3nc(C4CC4)cc32)cc1] |
| I-258 | 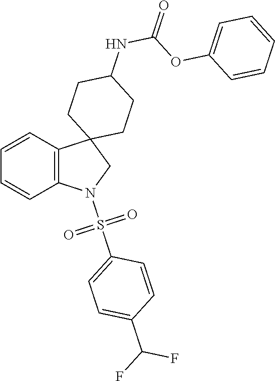 [SMILES: O=C(NC1CCC2(CC1)CN(S(=O)(=O)c1ccc(C(F)F)cc1)c1ccccc12)Oc1ccccc1] |
| I-259 | 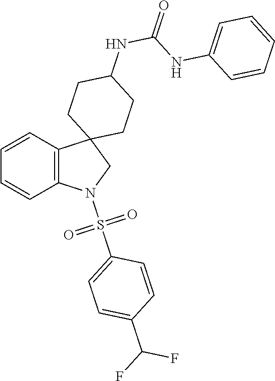 [SMILES: O=C(Nc1ccccc1)NC1CCC2(CC1)CN(S(=O)(=O)c1ccc(C(F)F)cc1)c1ccccc12] |
| I-260 | 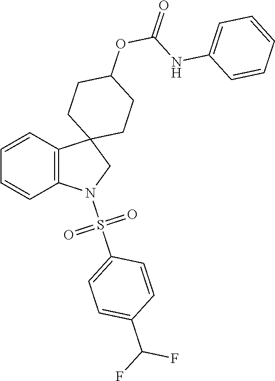 [SMILES: O=C(Nc1ccccc1)OC1CCC2(CC1)CN(S(=O)(=O)c1ccc(C(F)F)cc1)c1ccccc12] |
| I-261 | 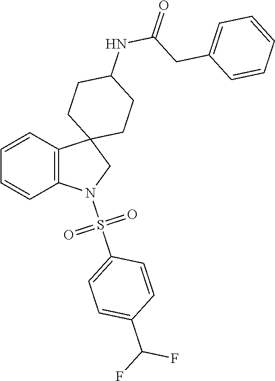 [SMILES: O=C(Cc1ccccc1)NC1CCC2(CC1)CN(S(=O)(=O)c1ccc(C(F)F)cc1)c1ccccc12] |
| I-262 | 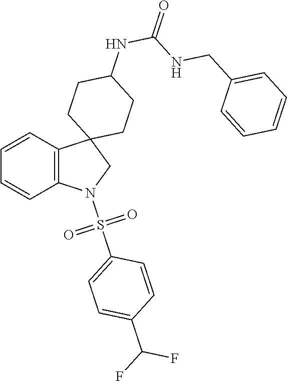 [SMILES: O=C(NCc1ccccc1)NC1CCC2(CC1)CN(S(=O)(=O)c1ccc(C(F)F)cc1)c1ccccc12] |
| I-263 | 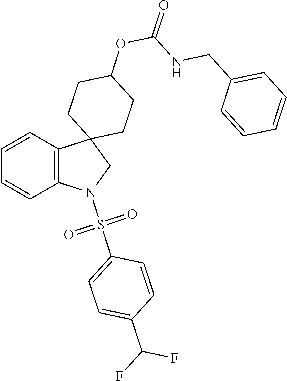 [SMILES: O=C(NCc1ccccc1)OC1CCC2(CC1)CN(S(=O)(=O)c1ccc(C(F)F)cc1)c1ccccc12] |
| I-264 | 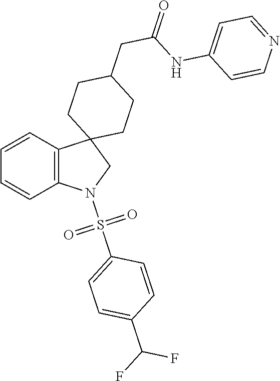 [SMILES: O=C(CC1CCC2(CC1)CN(S(=O)(=O)c1ccc(C(F)F)cc1)c1ccccc12)Nc1ccncc1] |
| I-265 | 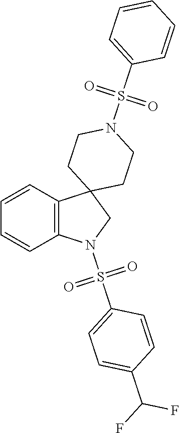 [SMILES: O=S(=O)(c1ccccc1)N1CCC2(CC1)CN(S(=O)(=O)c1ccc(C(F)F)cc1)c1ccccc12] |
| I-266 | 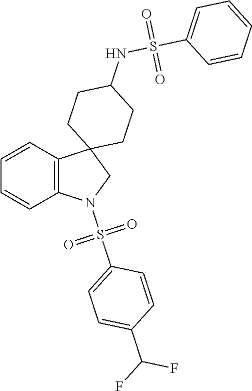 [SMILES: O=S(=O)(NC1CCC2(CC1)CN(S(=O)(=O)c1ccc(C(F)F)cc1)c1ccccc12)c1ccccc1] |
| I-267 | 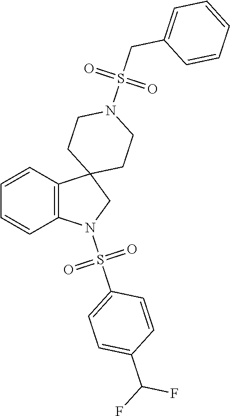 [SMILES: O=S(=O)(Cc1ccccc1)N1CCC2(CC1)CN(S(=O)(=O)c1ccc(C(F)F)cc1)c1ccccc12] |
| I-268 | 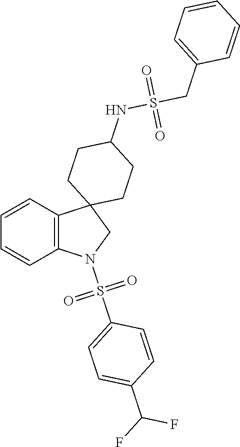 [SMILES: O=S(=O)(Cc1ccccc1)NC1CCC2(CC1)CN(S(=O)(=O)c1ccc(C(F)F)cc1)c1ccccc12] |

In some embodiments, the present disclosure provides a compound, or a pharmaceutically acceptable salt thereof, selected from Table 2:

TABLE 2

| Compound | |
|---|---|
| No. | Name |
| I-1 | 1′-phenylmethanesulfonyl-1′,2′-dihydrospiro[cyclopentane-1,3′-indole] |
| I-2 | 1′-[(2,2-dimethyl-2,3-dihydro-1-benzofuran-5-yl)sulfonyl]-1′,2′- |
| | dihydrospiro[cyclopentane-1,3′-indole] |
| I-3 | 1-[4-({1′,2′-dihydrospiro[cyclopentane-1,3′-indol]-1′-yl}sulfonyl)phenyl]-2- |
| | methylpropan-1-one |
| I-4 | 1′-[4-(propan-2-yl)benzenesulfonyl]-1′,2′-dihydrospiro[cyclopentane-1,3′-indole] |
| I-5 | 1′-[(3,3-dimethyl-2,3-dihydro-1-benzofuran-5-yl)sulfonyl]-1′,2′- |
| | dihydrospiro[cyclopentane-1,3′-indole] |
| I-6 | 6-({1′,2′-dihydrospiro[cyclopentane-1,3′-indol]-1′-yl}sulfonyl)-3-methyl-3,4- |
| | dihydro-2H-1,3-benzoxazin-2-one |
| I-7 | 1′-[2-(4-fluorophenyl)-2-methoxyethanesulfonyl]-1′,2′-dihydrospiro[cyclopentane- |
| | 1,3′-indole] |
| I-8 | 1′-[(2-methoxy-2,3-dihydro-1H-inden-5-yl)sulfonyl]-1′,2′- |
| | dihydrospiro[cyclopentane-1,3′-indole] |
| I-9 | 1′-(4-methanesulfonylbenzenesulfonyl)-1′,2′-dihydrospiro[cyclopentane-1,3′-indole] |
| I-10 | 1′-(3-fluoro-4-methanesulfonylbenzenesulfonyl)-1′,2′-dihydrospiro[cyclopentane- |
| | 1,3′-indole] |
| I-11 | 1′-(4-fluoro-3-methylbenzenesulfonyl)-1′,2′-dihydrospiro[cyclopentane-1,3′-indole] |
| I-12 | 1′-[(4-methylphenyl)methanesulfonyl]-1′,2′-dihydrospiro[cyclopentane-1,3′-indole] |
| I-13 | 1′-(4-methylbenzenesulfonyl)-1′,2′-dihydrospiro[cyclopentane-1,3′-indole] |
| I-14 | 1′-[(3,4-dimethylphenyl)methanesulfonyl]-1′,2′-dihydrospiro[cyclopentane-1,3′- |
| | indole] |
| I-15 | 1′-(3-fluoro-4-methylbenzenesulfonyl)-1′,2′-dihydrospiro[cyclopentane-1,3′-indole] |
| I-16 | 1′-[(3-methylphenyl)methanesulfonyl]-1′,2′-dihydrospiro[cyclopentane-1,3′-indole] |
| I-17 | 1′-[(1-cyclobutyl-1H-pyrazol-4-yl)sulfonyl]-1′,2′-dihydrospiro[cyclopentane-1,3′- |
| | indole] |
| I-18 | 1′-[(1-cyclohexyl-1H-pyrazol-4-y1)sulfonyl]-1′,2′-dihydrospiro[cyclopentane-1,3′ |
| | indole] |
| I-19 | 1′-{[]-(oxan-4-yl)-1H-pyrazol-4-yl]sulfonyl}-1′,2′-dihydrospiro[cyclopentane-1,3′- |
| | indole] |
| I-20 | 1′-{[S-(difluoromethyl)pyridin-3-yl]sulfonyl}-1′,2′-dihydrospiro[cyclopentane-1,3′- |
| | indole] |
| I-21 | 1′-{[4-(1,3-dioxolan-2-yl)thiophen-2-yl]sulfonyl}-1′,2′-dihydrospiro[cyclopentane- |
| | 1,3′-indole] |
| I-22 | 1′-[4-(1,3-dioxolan-2-yl)-2-fluorobenzenesulfonyl]-1′,2′-dihydrospiro[cyclopentane- |
| | 1,3′-indole] |
| I-23 | 1′-[4-(difluoromethoxy)-3-fluorobenzenesulfonyl]-1′,2′-dihydrospiro[cyclopentane- |
| | 1,3′-indole] |
| I-24 | 1′{[(1S,2R)-2-(3-fluorophenyl)cyclopropyl]sulfonyl}-1′,2′- |
| | dihydrospiro[cyclopentane-1,3′-indole] |
| I-25 | 1′-[(3-fluorophenyl)methanesulfonyl]-1′,2′-dihydrospiro[cyclopentane-1,3′-indole] |
| I-26 | 1′-(3-cyclopropylbenzenesulfonyl)-1′,2′-dihydrospiro[cyclopentane-1,3′-indole] |
| I-27 | 1′-[3-(1,3-dioxolan-2-yl)benzenesulfonyl]-1′,2′-dihydrospiro[cyclopentane-1,3′- |
| | indole] |
| I-28 | 1′-[4-(difluoromethyl)benzenesulfonyl]-1′,2′-dihydrospiro[cyclopentane-1,3′-indole] |
| I-29 | 1′-(4-cyclopropylbenzenesulfonyl)-1′,2′-dihydrospiro[cyclopentane-1,3′-indole] |
| I-30 | 1′-[(4-fluorophenyl)methanesulfonyl]-1′,2′-dihydrospiro[cyclopentane-1,3′-indole] |
| I-31 | 1′-[4-(trifluoromethoxy)benzenesulfonyl]-1′,2′-dihydrospiro[cyclopentane-1,3′- |
| | indole] |
| I-32 | 1′-(4-bromobenzenesulfonyl)-1′,2′-dihydrospiro[cyclopentane-1,3′-indole] |
| I-33 | 1′-[(5-cyclopropylthiophen-2-yl)sulfonyl]-1′,2′-dihydrospiro[cyclopentane-1,3′- |
| | indole] |
| I-34 | 1′-[(3,4-dihydro-1H-2-benzopyran-7-yl)methanesulfonyl]-1′,2′- |
| | dihydrospiro[cyclopentane-1,3′-indole] |
| I-35 | 1′-{[2-(difluoromethyl)-3,4-dihydro-2H-1-benzopyran-6-yl]sulfonyl}-1′,2′- |
| | dihydrospiro[cyclopentane-1,3′-indole] |
| I-36 | 1′-(1,3-dihydro-2-benzofuran-5-sulfonyl)-1′,2′-dihydrospiro[cyclopentane-1,3′- |
| | indole] |
| I-37 | 1′-(3,4-dihydro-1H-2-benzopyran-7-sulfonyl)-1′,2′-dihydrospiro[cyclopentane-1,3′- |
| | indolel |
| I-38 | 1′-(2-fluoro-2-phenylethanesulfonyl)-1′,2′-dihydrospiro[cyclopentane-1,3′-indole] |
| I-39 | 1′-[(2-phenyl-1,3-oxazol-5-yl)sulfonyl]-1′,2′-dihydrospiro[cyclopentane-1,3′-indole] |
| I-40 | 1′-(2,3-dihydro-1-benzoxepine-4-sulfonyl)-1′,2′-dihydrospiro[cyclopentane-1,3′- |
| | indole] |
| I-41 | 1′-(2H-chromene-3-sulfonyl)-1′,2′-dihydrospiro[cyclopentane-1,3′-indole] |
| I-42 | 1′-(3,4-dihydronaphthalene-2-sulfonyl)-1′,2′-dihydrospiro[cyclopentane-1,3′-indole] |
| I-43 | 1′-{[1-(2,2-difluorocyclopropyl)-1H-pyrazol-3-yl]sulfonyl}-1′,2′- |
| | dihydrospiro[cyclopentane-1,3′-indole] |
| I-44 | 1′-[(2-fluoro-4-methylphenyl)methanesulfonyl]-1′,2′-dihydrospiro[cyclopentane- |
| | 1,3′-indole] |
| I-45 | 1′-{[4-(difluoromethyl)phenyl]methanesulfonyl}-1′,2′-dihydrospiro[cyclopentane- |
| | 1,3′-indole] |
| I-46 | 1′-[(3-chlorophenyl)methanesulfonyl]-1′,2′-dihydrospiro[cyclopentane-1,3′-indole] |
| I-47 | 1′-[(2-fluoro-5-methylphenyl)methanesulfonyl]-1′,2′-dihydrospiro[cyclopentane- |
| | 1,3′-indole] |
| I-48 | 1′-[4-(2,2-difluorocyclopropyl)benzenesulfonyl]-1′,2′-dihydrospiro[cyclopentane- |
| | 1,3′-indole] |
| I-49 | 1′-[3-(2,2-difluorocyclopropyl)benzenesulfonyl]-1′,2′-dihydrospiro[cyclopentane- |
| | 1,3′-indole] |
| I-50 | 2-[4-({1′,2′-dihydrospiro[cyclopentane-1,3′-indol]-1′-yl}sulfonyl)phenyl]propan-2-ol |
| I-51 | 5-({1′,2′-dihydrospiro[cyclopentane-1,3′-indol]-1′-yl}sulfonyl)-2,2-dimethyl-2,3- |
| | dihydro-1H-inden-1-one |
| I-52 | N-[4-({1′,2′-dihydrospiro[cyclopentane-1,3′-indol]-1′-yl}sulfonyl)phenyl]-N- |
| | methylmethanesulfonamide |
| I-53 | methyl 2-[4-({1′,2′-dihydrospiro[cyclopentane-1,3′-indol]-1′-yl}sulfonyl)phenyl]-2- |
| | methylpropanoate |
| I-54 | 2-[4-({1′,2′-dihydrospiro[cyclopentane-1,3′-indol]-1′-yl}sulfonyl)phenyl]-2- |
| | methylpropan-1-ol |
| I-55 | 5-({1′,2′-dihydrospiro[cyclopentane-1,3′-indol]-1′-yl}sulfonyl)-2-methyl-2,3- |
| | dihydro-1H-isoindol-1-one |
| I-56 | 1′-[4-(2-methoxypropan-2-yl)benzenesulfonyl]-1′,2′-dihydrospiro[cyclopentane-1,3′- |
| | indole] |
| I-57 | 1′-[(2,2-dimethyl-2H-1,3-benzodioxol-5-yl)sulfony1]-1′,2′- |
| | dihydrospiro[cyclopentane-1,3′-indole] |
| I-58 | 1′-[(3,3-dimethyl-1,3-dihydro-2-benzofuran-5-yl)sulfonyl]-1′,2′- |
| | dihydrospiro[cyclopentane-1,3′-indole] |
| I-59 | 1′-(3,4-dihydro-1H-2-benzopyran-6-sulfonyl)-1′,2′-dihydrospiro[cyclopentane-1,3′- |
| | indole] |
| I-60 | 1′-[3-(2-methoxypropan-2-yl)benzenesulfonyl]-1′,2′-dihydrospiro[cyclopentane-1,3′- |
| | indole] |
| I-61 | 2-[3-({1′,2′-dihydrospiro[cyclopentane-1,3′-indol]-1′-yl}sulfonyl)phenyl]propan-2-ol |
| I-62 | methyl 4-[({1′,2′-dihydrospiro[cyclopentane-1,3′-indol]-1′- |
| | yl}sulfonyl)methyl]benzoate |
| I-63 | N-[4-({1′,2′-dihydrospiro[cyclopentane-1,3′-indol]-1′- |
| | yl}sulfonyl)phenyl]methanesulfonamide |
| I-64 | 1′-({spiro[1,3-benzodioxole-2,1′-cyclobutan]-6-yl}sulfonyl)-1′,2′- |
| | dihydrospiro[cyclopentane-1,3′-indole] |
| I-65 | 1′-(4-chloro-2-fluorophenyl)-1-(4-methanesulfonylbenzenesulfonyl)-1,2- |
| | dihydrospiro[indole-3,4′-piperidine] |
| I-66 | 1′-[(1,1-dimethyl-1,3-dihydro-2-benzofuran-5-yl)sulfonyl]-1′,2′- |
| | dihydrospiro[cyclopentane-1,3′-indole] |
| I-67 | 1′-(2,3-dihydro-1H-indene-5-sulfonyl)-1′,2′-dihydrospiro[cyclopentane-1,3′-indole] |
| I-68 | 4-({1′,2′-dihydrospiro[cyclopentane-1,3′-indol]-1′-yl}sulfonyl)-N,N- |
| | dimethylbenzene-1-sulfonamide |
| I-69 | 4-({1,2-dihydrospiro[indole-3,4′-oxan]-1-yl}sulfonyl)-N,N-dimethylbenzene-1- |
| | sulfonamide |
| I-70 | 4-({1′,2′-dihydrospiro[cyclopentane-1,3′-pyrrolo[3,2-c]pyridin]-1′-yl}sulfonyl)-N,N- |
| | dimethylbenzene-1-sulfonamide |
| I-71 | 4-({1′,2′-dihydrospiro[cyclopentane-1,3′-pyrrolo[2,3-b]pyridin]-1′-yl}sulfonyl)-N,N- |
| | dimethylbenzene-1-sulfonamide |
| I-72 | 4-({4-hydroxy-1′,2′-dihydrospiro[cyclohexane-1,3′-indol]-1′-yl}sulfonyl)-N,N- |
| | dimethylbenzene-1-sulfonamide |
| I-73 | 4-{[1′-(2,2-dimethylpropanoyl)-1,2-dihydrospiro[indole-3,3′-pyrrolidin]-1- |
| | yl]sulfonyl}-N,N-dimethylbenzene-1-sulfonamide |
| I-74 | 1′-(3-methylbenzenesulfonyl)-1′,2′-dihydrospiro[cyclopentane-1,3′-indole] |
| I-75 | 1′-{[1-(3-methylphenyl)-1H-pyrazol-4-yl]sulfonyl}-1′,2′-dihydrospiro[cyclopentane- |
| | 1,3′-indole] |
| I-76 | 4-({1,2-dihydrospiro[indole-3,3′-oxolan]-1-yl}sulfonyl)-N,N-dimethylbenzene-1- |
| | sulfonamide |
| I-77 | 4-({1,2-dihydrospiro[indole-3,2′-oxolan]-1-yl}sulfonyl)-N,N-dimethylbenzene-1- |
| | sulfonamide |
| I-78 | 4-({1′,2′-dihydrospiro[cyclopentane-1,3′-pyrrolo[3,2-b]pyridin]-1′-yl}sulfonyl)-N,N- |
| | dimethylbenzene-1-sulfonamide |
| I-79 | N,N-dimethyl-4-({6′-methyl-1′,2′-dihydrospiro[cyclopentane-1,3′-indol]-1′- |
| | yl}sulfonyl)benzene-1-sulfonamide |
| I-80 | benzyl 1-phenylmethanesulfonyl-1,2-dihydrospiro[indole-3,4′-piperidine]-1′- |
| | carboxylate |
| I-81 | benzyl 1-[4-(difluoromethyl)benzenesulfonyl]-1,2-dihydrospirofindole-3,4′- |
| | piperidine]-1′-carboxylate |
| I-82 | 1′-methyl-1-phenylmethanesulfonyl-1,2-dihydrospiro[indole-3,4′-piperidine] |
| I-83 | 1-[4-(difluoromethyl)benzenesulfonyl]-1′-methyl-1,2-dihydrospirofindole-3,4′- |
| | piperidine] |
| I-84 | N,N-dimethyl-4-({1′-methyl-1,2-dihydrospiro[indole-3,4′-piperidin]-1- |
| | yl}sulfonyl)benzene-1-sulfonamide |
| I-85 | 1-phenylmethanesulfonyl-1′-(2,2,2-trifluoroethyl)-1,2-dihydrospiro[indole-3,4′- |
| | piperidine] |
| I-86 | 1-[4-(difluoromethyl)benzenesulfonyl]-1′-(2,2,2-trifluoroethyl)-1,2- |
| | dihydrospiro[indole-3,4′-piperidine] |
| I-87 | N,N-dimethyl-4-{[1′-(2,2,2-trifluoroethyl)-1,2-dihydrospiro[indole-3,4′-piperidin]- |
| | 1-yl]sulfonyl}benzene-1-sulfonamide |
| I-88 | 1′-methyl-1-phenylmethanesulfonyl-1,2-dihydrospiro[indole-3,3′-pyrrolidine] |
| I-89 | 1-[4-(difluoromethyl)benzenesulfonyl]-1′-methyl-1,2-dihydrospirofindole-3,3′- |
| | pyrrolidine] |
| I-90 | N,N-dimethyl-4-({1′-methyl-1,2-dihydrospiro[indole-3,3′-pyrrolidin]-1- |
| | yl}sulfonyl)benzene-1-sulfonamide |
| I-91 | benzyl 1-[4-(difluoromethyl)benzenesulfonyl]-1,2-dihydrospirofindole-3,3′- |
| | pyrrolidine]-1′-carboxylate |
| I-92 | 1-[4-(difluoromethyl)benzenesulfonyl]-1′-(2,2,2-trifluoroethyl)-1,2- |
| | dihydrospiro[indole-3,3′-pyrrolidine] |
| I-93 | N,N-dimethyl-4-{[1′-(2,2,2-trifluoroethyl)-1,2-dihydrospiro[indole-3,3′-pyrrolidin]- |
| | 1-yl]sulfonyl }benzene-1-sulfonamide |
| I-94 | 1′-[(3,3-dimethyl-2,3-dihydro-1-benzofuran-5-yl)sulfony1]-1′,2′- |
| | dihydrospiro[cyclohexane-1,3′-indole] |
| I-95 | 4-({4′-fluoro-1′,2′-dihydrospiro[cyclopentane-1,3′-indol]-1′-yl}sulfonyl)-N,N- |
| | dimethylbenzene-1-sulfonamide |
| I-96 | N,N-dimethyl-4-({1′-[(oxan-4-yl)methyl]-1,2-dihydrospiro[indole-3,3′-pyrrolidin]- |
| | 1-yl}sulfonyl)benzene-1-sulfonamide |
| I-97 | 4-{[1′-(2,2-difluoropropyl)-1,2-dihydrospiro[indole-3,4′-piperidin]-1-yl]sulfonyl}- |
| | N,N-dimethylbenzene-1-sulfonamide |
| I-98 | N,N-dimethyl-4-({1′-[(oxan-4-yl)methyl]-1,2-dihydrospiro[indole-3,4′-piperidin]-1- |
| | yl }sulfonyl)benzene-1-sulfonamide |
| I-99 | 4-({1′,2′-dihydrospiro[cyclohexane-1,3′-indol]-1′-yl}sulfonyl)-N,N- |
| | dimethylbenzene-1-sulfonamide |
| I-100 | 1′-[(3,3-dimethyl-2,3-dihydro-1-benzofuran-5-yl)sulfonyl]-4′-fluoro-1′,2′- |
| | dihydrospiro[cyclopentane-1,3′-indole] |
| I-101 | N,N-dimethyl-4-({1-[(oxan-4-yl)methyl]-1′,2′-dihydrospiro[azetidine-3,3′-indol]-1′- |
| | yl}sulfonyl)benzene-1-sulfonamide |
| I-102 | 4-({1-benzyl-1′,2′-dihydrospiro[azetidine-3,3′-indol]-1′-yl}sulfonyl)-N,N- |
| | dimethylbenzene-1-sulfonamide |
| I-103 | N,N-dimethyl-4-{[1′-(oxolan-3-yl)-1,2-dihydrospiro[indole-3,4′-piperidin]-1- |
| | yl]sulfonyl}benzene-1-sulfonamide |
| I-104 | benzyl 1-[4-(dimethylsulfamoyl)benzenesulfonyl]-1,2-dihydrospirofindole-3,4′- |
| | piperidine]-1′-carboxylate |
| I-105 | benzyl 1′-[4-(dimethylsulfamoyl)benzenesulfonyl]-1′,2′-dihydrospiro[azetidine-3,3′- |
| | indole]-1-carboxylate |
| I-106 | 4-{[1′-(4-chloro-2-fluorophenyl)-1,2-dihydrospiro[indole-3,4′-piperidin]-1- |
| | yl]sulfonyl}-N,N-dimethylbenzene-1-sulfonamide |
| I-114 | methyl 2-[4-({1′,2′-dihydrospiro[cyclopentane-1,3′-indol]-1′-yl}sulfonyl)phenyl]-2- |
| | methylpropanoate |
| I-115 | benzyl 1-phenylmethanesulfonyl-1,2-dihydrospiro[indole-3,3′-pyrrolidine]-1′- |
| | carboxylate |
| I-116 | 4-((3,4-dihydrospiro[1,4-benzoxazine-2,1′-cyclopentan]-4-yl}sulfonyl)-N,N- |
| | dimethylbenzene-1-sulfonamide |
| I-117 | 1′-(2-phenylpropanesulfonyl)-1′,2′-dihydrospiro[cyclopentane-1,3′-indole] |
| I-118 | 4-{[1′-(2,2-dimethylpropanoyl)-1,2-dihydrospiro[indole-3,4′-piperidin]-1- |
| | yl]sulfonyl}-N,N-dimethylbenzene-1-sulfonamide |
| I-119 | 4-({4-methoxy-1′,2′-dihydrospiro[cyclohexane-1,3′-indol]-1′-yl}sulfonyl)-N,N- |
| | dimethylbenzene-1-sulfonamide |
| I-120 | 1-phenylmethanesulfonyl-1′-(2,2,2-trifluoroethyl)-1,2-dihydrospiro[indole-3,3′- |
| | pyrrolidinel |
| I-121 | 1′-(benzylsulfonyl)spiro[cyclohexane-1,3′-indoline] |
| I-122 | 1-((4-(difluoromethyl)phenyl)sulfonyl)-1′-(2,2-difluoropropyl)spiro[indoline-3,4′- |
| | piperidine] |
| I-123 | N,N-dimethyl-4-({6′-methyl-1′,2′-dihydrospiro[cyclohexane-1,3′-pyrazolo[1,5- |
| | alimidazol]-1′-yl}sulfonyl)benzene-1-sulfonamide |
| I-124 | 4-({1′,2′-dihydrospiro[cyclopentane-1,3′-pyrazolo[1,5-a]imidazol]-1′-yl}sulfonyl)- |
| | N,N-dimethylbenzene-1-sulfonamide |
| I-125 | 4-({1′,2′-dihydrospiro[cyclohexane-1,3′-pyrazolo[1,5-a]imidazol]-1′-yl}sulfonyl)- |
| | N,N-dimethylbenzene-1-sulfonamide |
| I-126 | N,N-dimethyl-4-{[1-(2,2,2-trifluoroethyl)-2′,3′-dihydro-1′H-spiro[piperidine-4,4′- |
| | quinolin]-1′-yl]sulfonyl}benzene-1-sulfonamide |
| I-127 | 4-({1′,2′-dihydrospiro[cyclopentane-1,3′-pyrrolo[2,3-c]pyridin]-1′-yl}sulfonyl)-N,N- |
| | dimethylbenzene-1-sulfonamide |
| I-128 | 4-({2′,3′-dihydro-1′H-spiro[cyclohexane-1,4′-quinolin]-1′-yl}sulfonyl)-N,N- |
| | dimethylbenzene-1-sulfonamide |
| I-129 | 4-({1′,2′-dihydrospiro[cyclohexane-1,3′-indol]-1′-yl}sulfonyl)-N,N- |
| | dimethylbenzene-1-sulfonoimidamide |
| I-130 | benzyl 1-((4-(difluoromethyl)phenyl)sulfonyl)-4-fluorospiro[indoline-3,4′- |
| | piperidine]-1′-carboxylate |
| I-131 | benzyl 1-((4-(difluoromethyl)phenyl)sulfonyl)-6-fluorospiro[indoline-3,4′- |
| | piperidine]-1′-carboxylate |
| I-132 | benzyl 1-((4-(difluoromethyl)phenyl)sulfonyl)-5-fluorospiro[indoline-3,4′- |
| | piperidine]-1′-carboxylate |
| I-133 | benzyl 1-((4-(difluoromethyl)phenyl)sulfonyl)-7-fluorospiro[indoline-3,4′- |
| | piperidine]-1′-carboxylate |
| I-134 | benzyl 1′-((4-(difluoromethyl)phenyl)sulfony1)-1′,2′-dihydrospiro[piperidine-4,3′- |
| | pyrrolo[3,2-b]pyridine]-1-carboxylate |
| I-135 | benzyl 1′-((4-(difluoromethyl)phenyl)sulfonyl)-1′,2′-dihydrospiro[piperidine-4,3′- |
| | pyrrolo[2,3-c]pyridine]-1-carboxylate |
| I-136 | (1-((4-(difluoromethyl)phenyl)sulfonyl)spiro[indoline-3,4′-piperidin]-1′- |
| | yl)(pyrrolidin-1-yl)methanone |
| I-137 | (1r,4r)-1′-((4-(difluoromethyl)phenyl)sulfonyl)-4-(ethoxymethyl)-4- |
| | methylspiro[cyclohexane-1,3′-indoline] |
| I-138 | 1′-((4-(difluoromethyl)phenyl)sulfonyl)-4,4-difluorospiro[cyclohexane-1,3′- |
| | indoline |
| I-139 | benzyl 1-((4-(cyclopropyldifluoromethyl)phenyl)sulfonyl)spiro[indoline-3,4′- |
| | piperidine]-1′-carboxylate |
| I-140 | benzyl 6-cyclopropyl-1-((4-(difluoromethyl)phenyl)sulfonyl)-1,2- |
| | dihydrospiro[imidazo[1,2-b]pyrazole-3,4′-piperidine]-1′-carboxylate |
| I-141 | pyridin-2-ylmethyl 1-((4-(difluoromethyl)phenyl)sulfonyl)spiro[indoline-3,4′- |
| | piperidine]-1′-carboxylate |
| I-142 | N-cyclopropyl-1-((4-(difluoromethyl)phenyl)sulfonyl)spiro[indoline-3,4′- |
| | piperidine]-1′-carboxamide |
| 1-143 | N-benzyl-1′-((4-(difluoromethyl)phenyl)sulfonyl)spiro[cyclohexane-1,3′-indoline]- |
| | 4-carboxamide |
| I-144 | N,N-dimethyl-4-{[1-(2,2,2-trifluoroethyl)-1′,2′-dihydrospiro[piperidine-4,3′- |
| | pyrazolo[1,5-a]imidazol]-1′-yl]sulfonyl}benzene-1-sulfonamide |
| I-145 | cyclopropylmethyl 1′-[4-(dimethylsulfamoyl)benzenesulfonyl]-1′,2′- |
| | dihydrospiro[piperidine-4,3′-pyrazolo[1,5-a]imidazole]-1-carboxylate |
| I-146 | N,N-dimethyl-4-([6′-methyl-1′,2′-dihydrospiro[cyclohexane-1,3′-pyrazolo[1,5- |
| | alimidazol]-1′-yl}sulfonyl)benzene-1-sulfonoimidamide |
| I-147 | 1′-[(3,3-dimethyl-2,3-dihydro-1-benzofuran-5-yl)sulfonyl]-6′-methyl-1′,2′- |
| | dihydrospiro[cyclohexane-1,3′-pyrazolo[1,5-a]imidazole] |
| I-148 | 1′-[4-(difluoromethyl)benzenesulfonyl]-6′-methyl-1′,2′-dihydrospiro[cyclohexane- |
| | 1,3′-pyrazolo[1,5-a]imidazole] |
| I-149 | N,N-dimethyl-4-({6′-methyl-1′,2′-dihydrospiro[cyclohexane-1,3′-indol]-1′- |
| | yl}sulfonyl)benzene-1-sulfonoimidamide |
| I-150 | 4-(1-{1′,2′-dihydrospiro[cyclohexane-1,3′-indol]-1′-yl}ethyl)-N,N-dimethylbenzene- |
| | 1-sulfonamide |
| I-151 | {cyclopropyl[4-({1′,2′-dihydrospiro[cyclohexane-1,3′-indol]-1′- |
| | yl}sulfonyl)phenyl]imino-λ⁶-sulfanyl}one |
| I-152 | 1-((4-(difluoromethyl)phenyl)sulfonyl)-2′,3′,5′,6′-tetrahydrospiro[indoline-3,4′- |
| | thiopyran] 1′,1′-dioxide |
| I-153 | N,N-dimethyl-4-{[6′-(trifluoromethyl)-1′,2′-dihydrospiro[cyclohexane-1,3′- |
| | pyrazolo[1,5-a]imidazol]-1′-yl]sulfonyl}benzene-1-sulfonamide |
| I-154 | 4-({6′,7′-dimethyl-1′,2′-dihydrospiro[cyclohexane-1,3′-pyrazolo[1,5-a]imidazol]-1′- |
| | yl}sulfonyl)-N,N-dimethylbenzene-1-sulfonamide |
| I-155 | 4-((1′-benzyl-4-fluorospiro[indoline-3,4′-piperidin]-1-yl)sulfonyl)-N,N- |
| | dimethylbenzenesulfonamide |
| I-157 | N,N-dimethyl-4-({6′-methyl-1′,2′-dihydrospiro[cyclohexane-1,3′-pyrrolo[3,2- |
| | b]pyridin]-1′-yl}sulfonyl)benzene-1-sulfonoimidamide |
| I-158 | (pyridin-3-yl)methyl 1-[4-(difluoromethyl)benzenesulfonyl]-1,2- |
| | dihydrospiro[indole-3,4′-piperidine]-1′-carboxylate |
| I-159 | pyridin-4-ylmethyl 1-((4-(difluoromethyl)phenyl)sulfonyl)spiro[indoline-3,4′- |
| | piperidine]-1′-carboxylate |
| I-160 | propan-2-yl 1-[4-(difluoromethyl)benzenesulfonyl]-1,2-dihydrospiro[indole-3,4′- |
| | piperidine]-1′-carboxylate |
| I-161 | 1′-benzoyl-1-[4-(difluoromethyl)benzenesulfonyl]-1,2-dihydrospiro[indole-3,4′- |
| | piperidine] |
| I-162 | 1′-cyclopropanecarbonyl-1-[4-(difluoromethyl)benzenesulfonyl]-1,2- |
| | dihydrospiro[indole-3,4′-piperidine] |
| I-163 | 1-[4-(difluoromethyl)benzenesulfonyl]-1′-[(pyrazin-2-yl)methyl]-1,2- |
| | dihydrospiro[indole-3,4′-piperidine] |
| I-164 | 1-[4-(difluoromethyl)benzenesulfonyl]-1′-[(pyridin-2-yl)methyl]-1,2- |
| | dihydrospiro[indole-3,4′-piperidine |
| I-165 | 1-[4-(difluoromethyl)benzenesulfonyl]-1′-[(pyridin-3-yl)methyl]-1,2- |
| | dihydrospiro[indole-3,4′-piperidine] |
| I-166 | 1-[4-(difluoromethyl)benzenesulfonyl]-1′-[(pyridin-4-yl)methyl]-1,2- |
| | dihydrospiro[indole-3,4′-piperidine] |
| I-167 | 1-[4-(difluoromethyl)benzenesulfonyl]-1′-[(pyrimidin-2-yl)methyl]-1,2- |
| | dihydrospiro[indole-3,4′-piperidine] |
| I-168 | 1′-benzyl-1-[4-(difluoromethyl)benzenesulfonyl]-1,2-dihydrospiro[indole-3,4′- |
| | piperidine] |
| I-169 | phenyl 1-((4-(difluoromethyl)phenyl)sulfonyl)spiro[indoline-3,4′-piperidine]-1′- |
| | carboxylate |
| I-170 | cyclopropylmethyl 1-((4-(difluoromethyl)phenyl)sulfonyl)spiro[indoline-3,4′- |
| | piperidine]-1′-carboxylate |
| I-171 | (1-ethyl-1H-pyrazol-4-yl)methyl 1-((4- |
| | (difluoromethyl)phenyl)sulfonyl)spirofindoline-3,4′-piperidine]-1′-carboxylate |
| I-172 | (1-methyl-1H-imidazol-4-yl)methyl 1-((4- |
| | (difluoromethyl)phenyl)sulfonyl)spiro[indoline-3,4′-piperidine]-1′-carboxylate |
| I-173 | isoxazol-4-ylmethyl 1-((4-(difluoromethyl)phenyl)sulfonyl)spiro[indoline-3,4′- |
| | piperidine]-1′-carboxylate |
| I-174 | 1-(1-((4-(difluoromethyl)phenyl)sulfonyl)spiro[indoline-3,4′-piperidin]-1′-y1)-3- |
| | phenylpropan-1-one |
| I-175 | 1-(1-((4-(difluoromethyl)phenyl)sulfonyl)spiro[indoline-3,4′-piperidin]-1′-yl)-2- |
| | phenylethan-1-one |
| I-176 | (1-methyl-1H-pyrazol-4-yl)methyl 1-((4- |
| | (difluoromethyl)phenyl)sulfonyl)spirofindoline-3,4′-piperidine]-1′-carboxylate |
| I-177 | (1-ethyl-1H-pyrazol-3-yl)methyl 1-((4- |
| | (difluoromethyl)phenyl)sulfonyl)spiro[indoline-3,4′-piperidine]-1′-carboxylate |
| 1-178 | N-benzyl-1-((4-(difluoromethyl)phenyl)sulfonyl)-N-methylspiro[indoline-3,4′- |
| | piperidine]-1′-carboxamide |
| I-179 | N-benzyl-1-((4-(difluoromethyl)phenyl)sulfonyl)spiro[indoline-3,4′-piperidine]-1′- |
| | carboxamide |
| I-180 | benzyl 1-(4-methanesulfonylbenzenesulfonyl)-1,2-dihydrospirofindole-3,4′- |
| | piperidine]-1′-carboxylate |
| I-181 | benzyl 1-[4-(propane-2-sulfonyl)benzenesulfonyl]-1,2-dihydrospiro[indole-3,4′- |
| | piperidine]-1′-carboxylate |
| I-182 | benzyl 1-((1,1-dioxido-2,3-dihydrobenzo[b]thiophen-5-yl)sulfonyl)spiro[indoline- |
| | 3,4′-piperidine]-1′-carboxylate |
| I-183 | benzyl 1-((1,3-dihydroisobenzofuran-5-yl)sulfonyl)spiro[indoline-3,4′-piperidine]- |
| | 1′-carboxylate |
| I-184 | benzyl 1-benzoyl-1,2-dihydrospiro[indole-3,4′-piperidine]-1′-carboxylate |
| I-185 | benzyl 1-(4-(difluoromethyl)benzoyl)spiro[indoline-3,4′-piperidine]-1′-carboxylate |
| I-186 | benzyl 1-((4-((difluoromethyl)sulfonyl)phenyl)sulfonyl)spiro[indoline-3,4′- |
| | piperidine]-1′-carboxylate |
| I-187 | benzyl 1-((6-(trifluoromethyl)pyridin-3-yl)sulfonyl)spiro[indoline-3,4′-piperidine]- |
| | 1′-carboxylate |
| I-188 | benzyl 1-((4-cyclopropylphenyl)sulfonyl)spiro[indoline-3,4′-piperidine]-1′- |
| | carboxylate |
| 1-189 | benzyl 1-((2,2-dimethyl-2,3-dihydrobenzofuran-5-yl)sulfonyl)spiro[indoline-3,4′- |
| | piperidine]-1′-carboxylate |
| I-190 | benzyl 1-(2-methylpropanoyl)-1,2-dihydrospiro[indole-3,4′-piperidine]-1′- |
| | carboxylate |
| I-191 | benzyl 1-(1-phenylcyclopropanecarbonyl)-1,2-dihydrospiro[indole-3,4′-piperidine]- |
| | 1′-carboxylate |
| I-192 | 1-((4-(difluoromethyl)phenyl)sulfonyl)-N-isopropylspiro[indoline-3,4′-piperidine]- |
| | 1′-carboxamide |
| I-193 | N-(cyclopropylmethyl)-1-((4-(difluoromethyl)phenyl)sulfonyl)spiro[indoline-3,4′- |
| | piperidine]-1′-carboxamide |
| I-194 | N-(2,2-difluoroethyl)-1-((4-(difluoromethyl)phenyl)sulfonyl)spiro[indoline-3,4′- |
| | piperidine]-1′-carboxamide |
| I-195 | 1-((4-(difluoromethyl)phenyl)sulfonyl)-N-(tetrahydrofuran-3-yl)spiro[indoline-3,4′- |
| | piperidine]-1′-carboxamide |
| I-196 | 1-((4-(difluoromethyl)phenyl)sulfonyl)-N-(2-ethoxyethyl)spiro[indoline-3,4′- |
| | piperidine]-1′-carboxamide |
| I-197 | 1-((4-(difluoromethyl)phenyl)sulfonyl)-N-phenylspiro[indoline-3,4′-piperidine]-1′- |
| | carboxamide |
| I-198 | 1-((4-(difluoromethyl)phenyl)sulfonyl)-N-(pyridin-3-yl)spiro[indoline-3,4′- |
| | piperidine]-1′-carboxamide |
| I-199 | 1-((4-(difluoromethyl)phenyl)sulfonyl)-N-(1-methyl-1H-pyrrol-2-yl)spiro[indoline- |
| | 3,4′-piperidine]-1′-carboxamide |
| I-200 | 1-((4-(difluoromethyl)phenyl)sulfonyl)-N-(1-methyl-1H-pyrazol-4- |
| | yl)spiro[indoline-3,4′-piperidine]-1′-carboxamide |
| I-201 | 1-((4-(difluoromethyl)phenyl)sulfonyl)-N-(2,2,2-trifluoroethyl)spiro[indoline-3,4′- |
| | piperidine]-1′-carboxamide |
| I-202 | 1-((4-(difluoromethyl)phenyl)sulfonyl)-N-(isothiazol-4-yl)spiro[indoline-3,4′- |
| | piperidine]-1′-carboxamide |
| I-203 | 1-((4-(difluoromethyl)phenyl)sulfonyl)-N-((tetrahydrofuran-2- |
| | yl)methyl)spiro[indoline-3,4′-piperidine]-1′-carboxamide |
| I-204 | 1-((4-(difluoromethyl)phenyl)sulfonyl)-N-((tetrahydrofuran-3- |
| | yl)methyl)spirofindoline-3,4′-piperidine]-1′-carboxamide |
| I-205 | 1-((4-(difluoromethyl)phenyl)sulfonyl)-N-(tetrahydro-2H-pyran-4-yl)spiro[indoline- |
| | 3,4′-piperidine]-1′-carboxamide |
| I-206 | N-(3,3-difluorocyclobutyl)-1-((4-(difluoromethyl)phenyl)sulfonyl)spiro[indoline- |
| | 3,4′-piperidine]-1′-carboxamide |
| I-207 | 1-((4-(difluoromethyl)phenyl)sulfonyl)-N-(1-ethyl-1H-pyrazol-4-yl)spiro[indoline- |
| | 3,4′-piperidine]-1′-carboxamide |
| I-208 | N-(2-(1H-pyrazol-1-yl)ethyl)-1-((4-(difluoromethyl)phenyl)sulfonyl)spiro[indoline- |
| | 3,4′-piperidine]-1′-carboxamide |
| I-209 | 1-((4-(difluoromethyl)phenyl)sulfonyl)-N-((1-methyl-1H-pyrazol-4- |
| | yl)methyl)spiro[indoline-3,4′-piperidine]-1′-carboxamide |
| I-210 | 1-((4-(difluoromethyl)phenyl)sulfonyl)-N-(5-fluoropyridin-3-yl)spiro[indoline-3,4′- |
| | piperidine]-1′-carboxamide |
| I-211 | 1-((4-(difluoromethyl)phenyl)sulfonyl)-N-((5-methylisoxazol-3- |
| | yl)methyl)spiro[indoline-3,4′-piperidine]-1′-carboxamide |
| I-212 | 1-((4-(difluoromethyl)phenyl)sulfonyl)-N-((3-methylisoxazol-5- |
| | yl)methyl)spirofindoline-3,4′-piperidine]-1′-carboxamide |
| I-213 | 1-((4-(difluoromethyl)phenyl)sulfonyl)-N-(3,5-dimethylisoxazol-4- |
| | yl)spiro[indoline-3,4′-piperidine]-1′-carboxamide |
| I-214 | 1-((4-(difluoromethyl)phenyl)sulfonyl)-N-(3,3,3-trifluoropropyl)spiro[indoline-3,4′- |
| | piperidine]-1′-carboxamide |
| I-215 | 1-((4-(difluoromethyl)phenyl)sulfonyl)-N-((tetrahydro-2H-pyran-4- |
| | yl)methyl)spiro[indoline-3,4′-piperidine]-1′-carboxamide |
| I-216 | 1-((4-(difluoromethyl)phenyl)sulfonyl)-N-(6-methoxypyridin-3-yl)spiro[indoline- |
| | 3,4′-piperidine]-1′-carboxamide |
| I-217 | 1-((4-(difluoromethyl)phenyl)sulfonyl)-N-(1,3,5-trimethyl-1H-pyrazol-4- |
| | yl)spiro[indoline-3,4′-piperidine]-1′-carboxamide |
| 1-218 | N-(4,4-difluorocyclohexyl)-1-((4-(difluoromethyl)phenyl)sulfonyl)spiro[indoline- |
| | 3,4′-piperidine]-1′-carboxamide |
| I-219 | 1-((4-(difluoromethyl)phenyl)sulfonyl)-N-(2,3-dihydrobenzofuran-5- |
| | yl)spiro[indoline-3,4′-piperidine]-1′-carboxamide |
| I-220 | N-(benzo[d][1,3]dioxol-5-yl)-1-((4-(difluoromethyl)phenyl)sulfonyl)spiro[indoline- |
| | 3,4′-piperidine]-1′-carboxamide |
| I-221 | N-(3-(difluoromethyl)phenyl)-1-((4-(difluoromethyl)phenyl)sulfonyl)spiro[indoline- |
| | 3,4′-piperidine]-1′-carboxamide |
| I-222 | N-(chroman-3-yl)-1-((4-(difluoromethyl)phenyl)sulfonyl)spiro[indoline-3,4′- |
| | piperidine]-1′-carboxamide |
| I-223 | N-(chroman-4-yl)-1-((4-(difluoromethyl)phenyl)sulfonyl)spiro[indoline-3,4′- |
| | piperidine]-1′-carboxamide |
| I-224 | 1-((4-(difluoromethyl)phenyl)sulfonyl)-N-(1,1-dioxidotetrahydro-2H-thiopyran-4- |
| | yl)spiro[indoline-3,4′-piperidine]-1′-carboxamide |
| I-225 | 1-((4-(difluoromethyl)phenyl)sulfonyl)-N-(2,3-dihydrobenzo[b][1,4]dioxin-6- |
| | yl)spiro[indoline-3,4′-piperidine]-1′-carboxamide |
| I-226 | 1-((4-(difluoromethyl)phenyl)sulfonyl)-N-(3,4-dimethoxyphenyl)spiro[indoline- |
| | 3,4′-piperidine]-1′-carboxamide |
| I-227 | 1-((4-(difluoromethyl)phenyl)sulfonyl)-N-(2,4-dimethoxyphenyl)spiro[indoline- |
| | 3,4′-piperidine]-1′-carboxamide |
| I-228 | 1-((4-(difluoromethyl)phenyl)sulfonyl)-N-(2,3-dimethoxyphenyl)spiro[indoline- |
| | 3,4′-piperidine]-1′-carboxamide |
| I-229 | 1-((4-(difluoromethyl)phenyl)sulfonyl)-N-(1,3-dimethyl-2,4-dioxo-1,2,3,4- |
| | tetrahydropyrimidin-5-yl)spiro[indoline-3,4′-piperidine]-1′-carboxamide |
| I-230 | 1′-((4-(difluoromethyl)phenyl)sulfonyl)-N-phenylspiro[cyclohexane-1,3′-indoline]- |
| | 4-carboxamide |
| I-231 | 1′-((4-(difluoromethyl)phenyl)sulfonyl)-N-(pyridin-2-ylmethyl)spiro[cyclohexane- |
| | 1,3′-indoline]-4-carboxamide |
| I-232 | 1′-((4-(difluoromethyl)phenyl)sulfonyl)-N-(pyridin-3-ylmethyl)spiro[cyclohexane- |
| | 1,3′-indoline]-4-carboxamide |
| I-233 | 1′-((4-(difluoromethyl)phenyl)sulfonyl)-N-(pyridin-4-ylmethyl)spiro[cyclohexane- |
| | 1,3′-indoline]-4-carboxamide |
| I-234 | 1′-((4-(difluoromethyl)phenyl)sulfonyl)-N-methyl-N-phenylspiro[cyclohexane-1,3′- |
| | indoline]-4-carboxamide |
| I-235 | N-benzyl-1′-((4-(difluoromethyl)phenyl)sulfonyl)-N-methylspiro[cyclohexane-1,3′- |
| | indoline]-4-carboxamide |
| I-236 | N-(cyclopropylmethyl)-1′-((4-(difluoromethyl)phenyl)sulfonyl)spiro[cyclohexane- |
| | 1,3′-indoline]-4-carboxamide |
| I-237 | (1′-((4-(difluoromethyl)phenyl)sulfonyl)spiro[cyclohexane-1,3′-indolin]-4- |
| | yl)(pyrrolidin-1-yl)methanone |
| I-238 | (1′-((4-(difluoromethyl)phenyl)sulfonyl)spiro[cyclohexane-1,3′-indolin]-4- |
| | yl)(morpholino)methanone |
| I-239 | (1′-((4-(difluoromethyl)phenyl)sulfonyl)spiro[cyclohexane-1,3′-indolin]-4-yl)(4,4- |
| | difluoropiperidin-1-yl)methanone |
| I-240 | (1′-((4-(difluoromethyl)phenyl)sulfonyl)spiro[cyclohexane-1,3′-indolin]-4-y1)(2- |
| | oxa-7-azaspiro[4.4]nonan-7-yl)methanone |
| I-241 | 1′-((4-(difluoromethyl)phenyl)sulfonyl)-N-((1-ethyl-1H-pyrazol-4- |
| | yl)methyl)spiro[cyclohexane-1,3′-indoline]-4-carboxamide |
| I-242 | 1′-((4-(difluoromethyl)phenyl)sulfonyl)-N-(isoxazol-3-ylmethyl)-N- |
| | methylspiro[cyclohexane-1,3′-indoline]-4-carboxamide |
| I-243 | 1′-((4-(difluoromethyl)phenyl)sulfonyl)-N-methyl-N-(1-methyl-1H-pyrazol-5- |
| | yl)spiro[cyclohexane-1,3′-indoline]-4-carboxamide |
| I-244 | (1′-((4-(difluoromethyl)phenyl)sulfonyl)spiro[cyclohexane-1,3′-indolin]-4-yl)(2- |
| | oxa-6-azaspiro[3.4]octan-6-yl)methanone |
| I-245 | 1′-((4-(difluoromethyl)phenyl)sulfonyl)-N-methyl-N-(1-methyl-1H-imidazol-2- |
| | yl)spiro[cyclohexane-1,3′-indoline]-4-carboxamide |
| I-246 | benzyl 1-(2-(4-(difluoromethyl)phenyl)acetyl)spiro[indoline-3,4′-piperidine]-1′- |
| | carboxylate |
| I-247 | benzyl 1-((4-(difluoromethyl)phenyl)carbamoyl)spiro[indoline-3,4′-piperidine]-1′- |
| | carboxylate |
| I-248 | benzyl 1-((4-(difluoromethyl)phenyl)(methyl)carbamoyl)spiro[indoline-3,4′- |
| | piperidine]-1′-carboxylate |
| I-249 | 1′-benzyl 1-(4-(difluoromethyl)phenyl) spiro[indoline-3,4′-piperidine]-1,1′- |
| | dicarboxylate |
| I-250 | benzyl 1-((4-(difluoromethyl)benzyl)sulfonyl)spiro[indoline-3,4′-piperidine]-1′- |
| | carboxylate |
| I-251 | 1-(1-((4-(difluoromethyl)phenyl)sulfonyl)spiro[indoline-3,4′-piperidin]-1′-y1)-4,4,4- |
| | trifluorobutan-1-one |
| I-252 | 1-(1-((4-(difluoromethyl)phenyl)sulfonyl)spiro[indoline-3,4′-piperidin]-1′-yl)-3- |
| | (tetrahydrofuran-2-yl)propan-1-one |
| I-253 | 1-(1-((4-(difluoromethyl)phenyl)sulfonyl)spiro[indoline-3,4′-piperidin]-1′-y1)-3- |
| | (tetrahydrofuran-3-yl)propan-1-one |
| I-254 | 1-(1-((4-(difluoromethyl)phenyl)sulfonyl)spiro[indoline-3,4′-piperidin]-1′-yl)-2- |
| | ((tetrahydrofuran-3-yl)oxy)ethan-1-one |
| I-255 | 1-(1-((4-(difluoromethyl)phenyl)sulfonyl)spiro[indoline-3,4′-piperidin]-1′-yl)-2- |
| | (tetrahydrofuran-3-yl)ethan-1-one |
| I-256 | 1-(1-((4-(difluoromethyl)phenyl)sulfonyl)spiro[indoline-3,4′-piperidin]-1′-y1)-2- |
| | (tetrahydrofuran-2-yl)ethan-1-one |
| I-257 | 4-({6′-cyclopropyl-1′,2′-dihydrospiro[cyclohexane-1,3′-pyrazolo[1,5-a]imidazol]-1′- |
| | yl}sulfony1)-N,N-dimethylbenzene-1-sulfonamide |
| I-258 | phenyl (1′-((4-(difluoromethyl)phenyl)sulfonyl)spiro[cyclohexane-1,3′-indolin]-4- |
| | yl)carbamate |
| I-259 | 1-(1′-((4-(difluoromethyl)phenyl)sulfonyl)spiro[cyclohexane-1,3′-indolin]-4-yl)-3- |
| | phenylurea |
| I-260 | 1′-((4-(difluoromethyl)phenyl)sulfonyl)spiro[cyclohexane-1,3′-indolin]-4-yl |
| | phenylcarbamate |
| I-261 | N-(1′-((4-(difluoromethyl)phenyl)sulfonyl)spiro[cyclohexane-1,3′-indolin]-4-y1)-2- |
| | phenylacetamide |
| I-262 | 1-benzyl-3-(1′-((4-(difluoromethyl)phenyl)sulfonyl)spiro[cyclohexane-1,3′-indolin]- |
| | 4-yl)urea |
| I-263 | 1′-((4-(difluoromethyl)phenyl)sulfonyl)spiro[cyclohexane-1,3′-indolin]-4-yl |
| | benzylcarbamate |
| I-264 | 2-(1′-((4-(difluoromethyl)phenyl)sulfonyl)spiro[cyclohexane-1,3′-indolin]-4-yl)-N- |
| | (pyridin-4-yl)acetamide |
| I-265 | 1-((4-(difluoromethyl)phenyl)sulfonyl)-1′-(phenylsulfonyl)spiro[indoline-3,4′- |
| | piperidine] |
| I-266 | N-(1′-((4-(difluoromethyl)phenyl)sulfonyl)spiro[cyclohexane-1,3′-indolin]-4- |
| | yl)benzenesulfonamide |
| I-267 | 1′-(benzylsulfonyl)-1-((4-(difluoromethyl)phenyl)sulfonyl)spiro[indoline-3,4′- |
| | piperidine] |
| I-268 | N-(1′-((4-(difluoromethyl)phenyl)sulfonyl)spiro[cyclohexane-1,3′-indolin]-4-yl)-1- |
| | phenylmethanesulfonamide |

### Characteristics

Among other things, in some embodiments, the present disclosure describes one or more characteristics of certain TRPML (e.g., TRPML1, TRPML2, and/or TRPML3) modulators provided by and/or useful in the practice of the present disclosure.

In some embodiments, the present disclosure provides technologies for assessing one or more relevant characteristics and/or for identifying, selecting, prioritizing, and/or characterizing one or more useful TRPML (e.g., TRPML1, TRPML2, and/or TRPML3) modulators.

In some embodiments, the present disclosure provides certain biological and/or chemical assays (e.g., that facilitate and/or permit assessment of one or more feature(s) of TRMPL (e.g., TRPML1, TRPML2, and/or TRPML3) expression and/or activity, and/or of impact of TRPML (e.g., TRPML1, TRPML2, and/or TRPML3) modulator(s) on such expression and/or activity. Alternatively or additionally, the present disclosure provides technologies for identifying and/or characterizing one or more aspects of biological pathway(s) (e.g., autophagy pathway(s)) involving TRMPL (e.g., TRPML1, TRPML2, and/or TRPML3), and thus permits identification and/or characterization of additional useful targets within such pathway(s) and/or of modulator(s) that impact such pathway(s) (whether or not targeting TRPML (e.g., TRPML1, TRPML2, and/or TRPML3) itself).

### Compositions

In some embodiments, the present disclosure provides and/or utilizes a composition that comprises and/or delivers a compound as described herein (e.g., together with one or more other components).

In some embodiments, the present disclosure provides compositions that comprise and/or deliver compounds reported herein (e.g., compounds of Formula I-IVb), or an intermediate, degradant, or an active metabolite thereof, e.g., when contacted with or otherwise administered to a system or environment e.g., which system or environment may include TRPML (e.g., TRPML1, TRPML2, and/or TRPML3) activity; in some embodiments, administration of such a composition to the system or environment achieves the regulation of autophagy and lysosomal biogenesis as described herein.

In some embodiments, a provided composition as described herein may be a pharmaceutical composition in that it comprises an active agent (e.g., compounds of Formula I-IVb) and one or more pharmaceutically acceptable excipients (e.g., one or more pharmaceutically acceptable adjuvants, carriers, excipients, and/or vehicles); in some such embodiments, a provided pharmaceutical composition comprises and/or delivers a compound described herein (e.g., compounds of Formula I-IVb), or an active metabolite thereof to a relevant system or environment (e.g., to a subject in need thereof) as described herein.

In some embodiments, a provided composition (e.g., a pharmaceutical composition) includes a compound (e.g., as described herein) in a salt form such as a pharmaceutically acceptable salt form.

Is some embodiments, a provided composition (e.g., a pharmaceutical composition) may be formulated for administration to a subject (e.g., a human) according to a particular route (e.g., orally, parenterally, by inhalation or nasal spray, topically (e.g., as by powders, ointments, or drops), rectally, buccally, intravaginally, intraperitoneally, intracisternally or via an implanted reservoir, etc).

In some embodiments, a provided composition (e.g., a pharmaceutical composition) comprises or delivers an amount of a compound as described herein (or an active metabolite thereof) that is effective to measurably modulate TRPML (e.g., TRPML1, TRPML2, and/or TRPML3) activity, and/or to induce autophagy and/or lysosomal biogenesis in a biological sample or in a subject, when administered in accordance with a therapeutic regimen.

In certain embodiments, a provided compound or composition is formulated for administration to a patient in need of such composition. In some embodiments, a compound or composition as described herein may be administered in a dose amount and/or by a route of administration effective for treating or lessening the severity of a disease or disorder described herein.

In some embodiments, a composition (e.g., a pharmaceutical composition) as described herein may be formulated in unit form (e.g., which may offer ease of administration and/or uniformity of dosage).

Those skilled in the art will appreciate that effective dose amounts may vary from subject to subject, for example depending on a variety of factors, including the disorder being treated and the severity of the disorder; the activity of the specific compound employed; the specific composition employed and its route of administration; the species, age, body weight, sex and diet of the patient; the general condition of the subject; the time of administration; the rate of excretion of the specific compound employed; the duration of the treatment; drugs used in combination or coincidental with the specific compound employed, and the like.

In some embodiments, an appropriate dosage level may be within a range of about 0.01 mg/kg to about 50 mg/kg, of subject body weight per day, one or more times a day, to obtain the desired therapeutic effect.

### Applications and Uses

The present application provides a variety of uses and applications for compounds and/or compositions as described herein, for example in light of their activities and/or characteristics as described herein. In some embodiments, such uses may include therapeutic and/or diagnostic uses. Alternatively, in some embodiments such uses may include research, production, and/or other technological uses.

Among other things, in some embodiments, the present disclosure provides technologies for modulating TRPML activity (e.g., TRPML1, TRPML2, TRPML3, or combinations thereof). In some embodiments, the present application relates to a method of modulating TRPML activity (e.g., TRPML1, TRPML2, TRPML3, or combinations thereof) in a subject comprising administering to the subject a provided compound, or a composition as described herein. In some embodiments, the present application relates to a method of modulating TRPML1 activity in a subject comprising administering to the subject a provided compound, or a composition as described herein. In some embodiments, the present application relates to a method of modulating TRPML2 activity in a subject comprising administering to the subject a provided compound, or a composition as described herein. In some embodiments, the present application relates to a method of modulating TRPML3 activity in a subject comprising administering to the subject a provided compound, or a composition as described herein. In some embodiments, the present application relates to a method of modulating TRPML1, TRPML2, and/or TRPML3 activity in a subject comprising administering to the subject a provided compound, or a composition as described herein.

### Diseases, Disorders, and Conditions

The present disclosure demonstrates that compounds and/or compositions as described herein may be useful in medicine (e.g., in the treatment of one or more diseases, disorders, or conditions).

Among other things, as described herein, the present disclosure provides an insight that targeting (e.g., agonizing) TRPML (e.g., TRPML1, TRPML2, TRPML3, or combinations thereof) may be a particularly effective strategy for modulating (e.g., enhancing) autophagy and/or lysosomal biogenesis.

In some embodiments, a disease, disorder or condition that may be treated as described herein may be or comprise a disease, disorder or condition associated with TRPML (e.g., TRPML1, TRPML2, TRPML3, or combinations thereof) deficiency. Furthermore, in some embodiments, the present disclosure identifies that TRMPL (e.g., TRPML1, TRPML2, TRPML3, or combinations thereof) deficiency is associated with particular diseases, disorders or conditions, some or all of which may be treated in accordance with the present disclosure.

In some embodiments, treatment provided herein involves administration of a TRMPL1 modulator as described herein in an amount effective to modulate TRMPL (e.g., TRPML1, TRPML2, TRPML3, or combinations thereof) activity in a lysosome and/or increase autophagy.

In some embodiments, a disease, disorder, or condition amenable to treatment as described herein is or comprises a liver disease, a neurodegenerative disorder, cancer, or a heart disease.

In some embodiments, a disease, disorder, or condition amenable to treatment as described herein is or comprises a lysosomal storage disease, such as Niemann-Pick C (NPC) disease, Gaucher disease, and Pompe disease.

In some embodiments, a disease, disorder, or condition amenable to treatment as described herein is an age-related common neurodegenerative disease, such as Alzheimer's Disease, Parkinson's Disease, and Huntington's Disease.

In some embodiments, a disease, disorder, or condition amenable to treatment as described herein is a type IV Mucolipidosis (ML4) neurodegenerative lysosomal storage disease caused by mutations in TRPML (e.g., TRPML1, TRPML2, TRPML3, or combinations thereof).

In some embodiments, a disease, disorder, or condition amenable to treatment as described herein is related to reactive oxygen species or oxidative stress.

In some embodiments, the present application relates to use of a compound and/or composition described herein for use in the manufacture of a medicament e.g., for modulation of TRPML (e.g., TRPML1, TRPML2, TRPML3, or combinations thereof) activity.

In some embodiments, the present application relates to use of a compound and/or composition described herein for use in the manufacture of a medicament for treating a disease, disorder or condition, e.g., through modulation of TRPML (e.g., TRPML1, TRPML2, TRPML3, or combinations thereof) activity; in some embodiments, the disease, disorder, or condition is a liver disease, a neurodegenerative disorder, cancer, or a heart disease.

In some embodiments, a disease, disorder, or condition is a muscular disease, a liver disease, a metabolic disease, an atherosclerotic disease, an inflammatory bowel disease, an atherosclerotic disease, a neurodegenerative disease, or an oncological disease. In some embodiments, a disease, disorder, or condition is a muscular disease. In some embodiments, a muscular disease is a muscular dystrophy. In some embodiments, a muscular dystrophy is Duchenne muscular dystrophy.

In some embodiments, a disease, disorder, or condition is an infectious disease. In some embodiments, an infectious disease is an infection of Helicobacter pylori or Mycobacterium tuberculosis.

In some embodiments, a disease, disorder, or condition is tuberculosis.

### EXEMPLARY EMBODIMENTS

The following numbered embodiments, while non-limiting, are exemplary of certain aspects of the disclosure:
Embodiment 1. A compound of formula I:

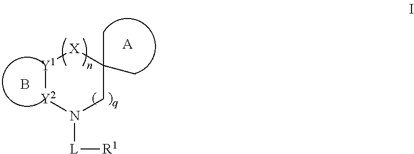

or a pharmaceutically acceptable salt thereof,
wherein
X is —NR⁵—, —C(R⁵)₂—, —C(O)—, or —O—;
each of Y¹ and Y² is independently selected from N and C;
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
B is a fused optionally substituted C₅-C₆ aryl or optionally substituted 5- to 6-membered heteroaryl comprising 1 to 3 heteroatoms selected from N, O, and S;
R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
each R² is independently halo, oxo, —NR^{2a}R^{2b}, —C(O)O—R^{2a}, —O—C(O)R², —S(O)₂, —S(O)₂—R^{2a}, —C(O)—NR^{2a}R^{2b}, —N(R^{2a})—C(O)—R^{2b}, —C(O)—R^{2a}, —O—R^{2b}, —O—C(O)—NR^{2a}R^{2b}, —NH—C(O)—NR²R^{2b}, —NH—C(O)—OR^{2a}, —NH—S(O)₂—R², —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R² and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3c})(O)—NR^{3a}R^{3b}, —S(O)(NR^{3c})—R^{3b}, —S(O)—R^{3b}, —NR^{3a}_S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{C} is independently selected from H, —OH, and optionally substituted C₁-C₆ aliphatic;
each R⁵ is independently selected from hydrogen, halo, —CN, and optionally substituted C₁-C₆ aliphatic;
R⁸ is selected from H and optionally substituted C₁-C₆ aliphatic;
n is 0 or 1;
m is 0 to 4;
p is 0 to 4; and
q is 1 or 2.
Embodiment 2. The compound of Embodiment 1, wherein n is 0.
Embodiment 3. The compound of Embodiments 1 or 2, wherein q is 1.
Embodiment 4. The compound of any one of Embodiments 1-3, wherein L is optionally substituted —S(O)₂—C₀-C₆ alkylenyl.
Embodiment 5. The compound of Embodiment 4, wherein L is —S(O)₂—.
Embodiment 6. The compound of Embodiment 4, wherein L is optionally substituted —S(O)₂—C₁-C₆ alkylenyl.
Embodiment 7. The compound of Embodiment 6, wherein L is —S(O)₂—CH₂—.
Embodiment 8. The compound of Embodiment 1, wherein L is selected from —S(O)₂—, —S(O)₂—CH₂—, —S(O)₂—CH(CH₃)—, —CH(CH₃)—S(O)₂—, —CH₂—S(O)₂—,

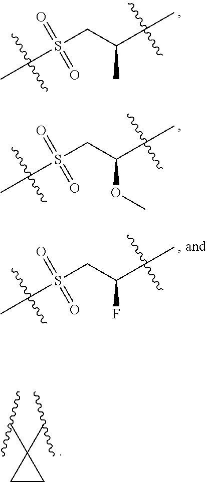

Embodiment 9. The compound of any one of Embodiments 1-8, wherein A is 3- to 12-membered heterocyclyl comprising 1 to 3 heteroatoms selected from N, O, and S.
Embodiment 10. The compound of any one of Embodiments 1-9, wherein A is 4- to 6-membered heterocyclyl comprising 1 to 3 heteroatoms selected from N, O, and S.
Embodiment 11. The compound of any one of Embodiments 1-10, wherein A is pyrrolidinyl or piperidinyl.
Embodiment 12. The compound of any one of Embodiments 1-8, wherein A is C₃-C₁₂ cycloaliphatic.
Embodiment 13. The compound of any one of Embodiments 1-8 or 12, wherein A is C₅-C₁₀ cycloaliphatic.
Embodiment 14. The compound of any one of Embodiments 1-8 or 12-13, wherein A is cyclopentyl or cyclohexyl.
Embodiment 15. The compound of Embodiment 1, wherein A is selected from:

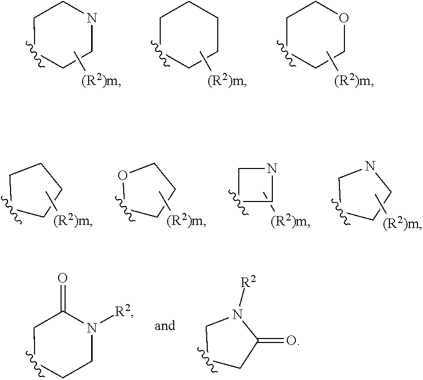

Embodiment 16. The compound of Embodiment 15, wherein A is selected from:

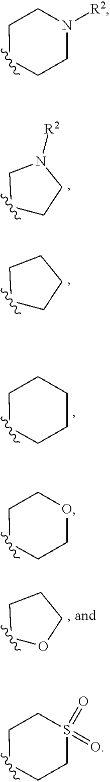

Embodiment 17. The compound of any one of Embodiments 1-16, wherein B is a fused optionally substituted C₅-C₆ aryl.
Embodiment 18. The compound of any one of Embodiments 1-16, wherein B is a fused optionally substituted 5- to 12-membered heteroaryl comprising 1 to 3 heteroatoms selected from N, O, and S.
Embodiment 19. The compound of Embodiment 18, wherein B is a fused optionally substituted 5- to 6-membered heteroaryl comprising 1 to 3 heteroatoms selected from N, O, and S.
Embodiment 20. The compound of any one of Embodiments 1-19, wherein R¹ is C₅-C₁₂ aryl substituted with (R³)ₚ, 5- to 12-membered heteroaryl comprising 1 to 3 heteroatoms selected from N, O, and S substituted with (R³)ₚ, or 3- to 12-membered heterocyclyl comprising 1 to 3 heteroatoms selected from N, O, and S substituted with (R³)ₚ.
Embodiment 21. The compound of any one of Embodiments 1-20, wherein R¹ is C₅-C₁₂ aryl substituted with (R³)P.
Embodiment 22. The compound of Embodiment 21, wherein R¹ is phenyl substituted with (R³)ₚ.
Embodiment 23. The compound of any one of Embodiments 1-22, wherein R¹ is 3- to 12-membered heterocyclyl comprising 1 to 3 heteroatoms selected from N, O, and S substituted with (R³)ₚ.
Embodiment 24. The compound of Embodiment 23, wherein R^{†} is bicyclic 10- to 12-membered heterocyclyl comprising 1 to 3 heteroatoms selected from N, O, and S substituted with (R³)ₚ.
Embodiment 25. The compound of any one of Embodiments 1-22, wherein R¹ is 5- to 12-membered heteroaryl comprising 1 to 3 heteroatoms selected from N, O, and S substituted with (R³)ₚ.
Embodiment 26. The compound of Embodiment 25, wherein R¹ is 5- to 6-membered heteroaryl comprising 1 to 3 heteroatoms selected from N, O, and S substituted with (R³)ₚ.
Embodiment 27. The compound of Embodiment 1, wherein R¹ is selected from —CH(CH₃)₂,

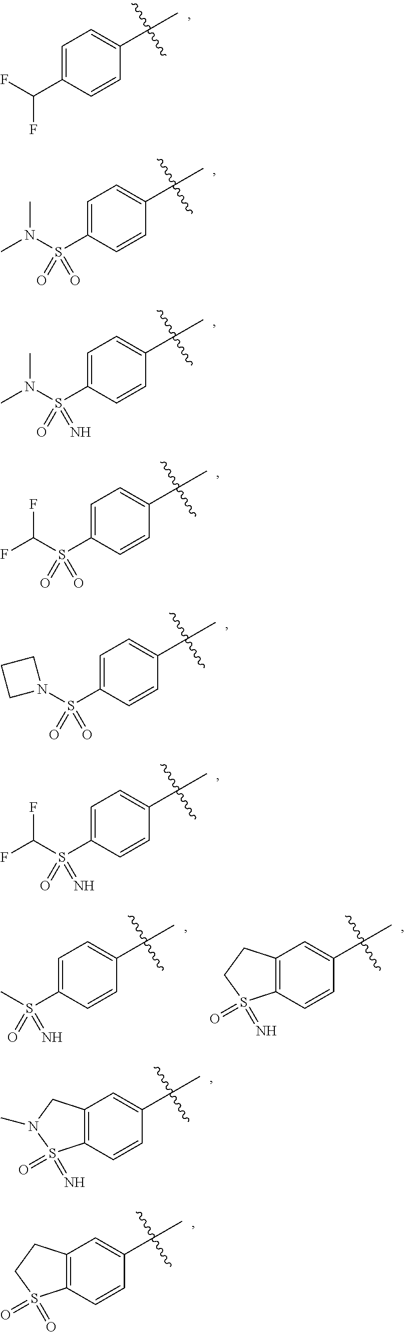

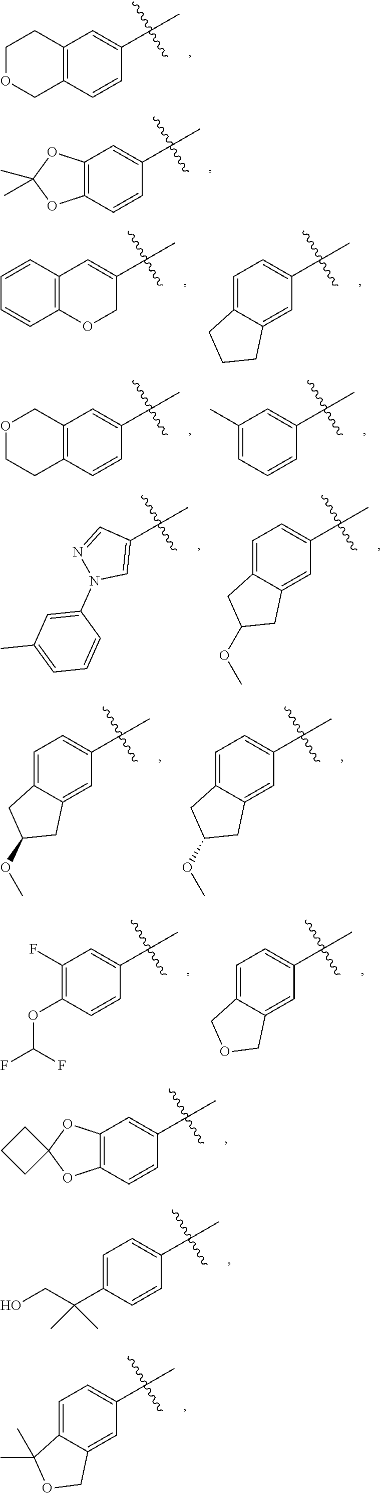

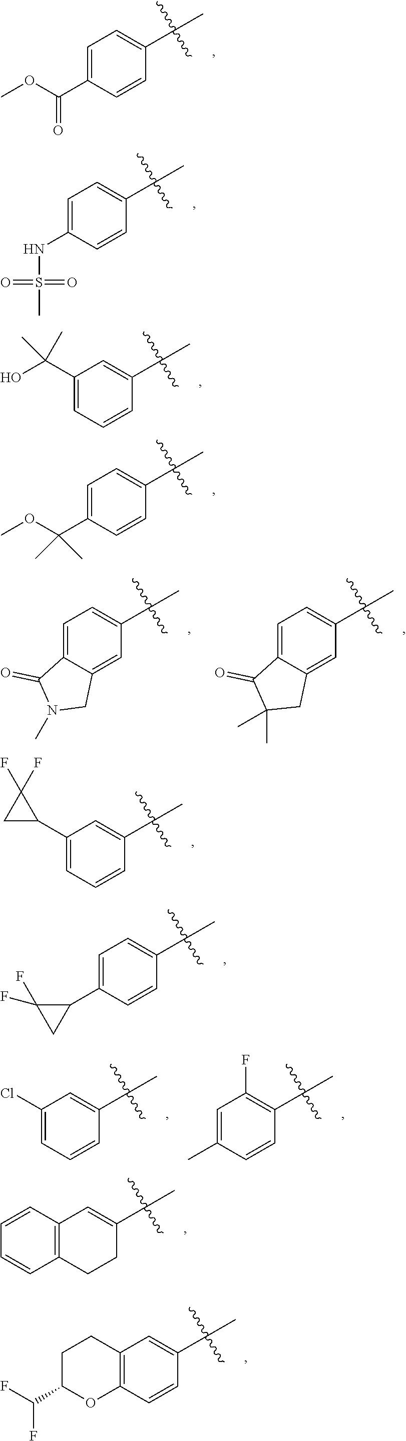

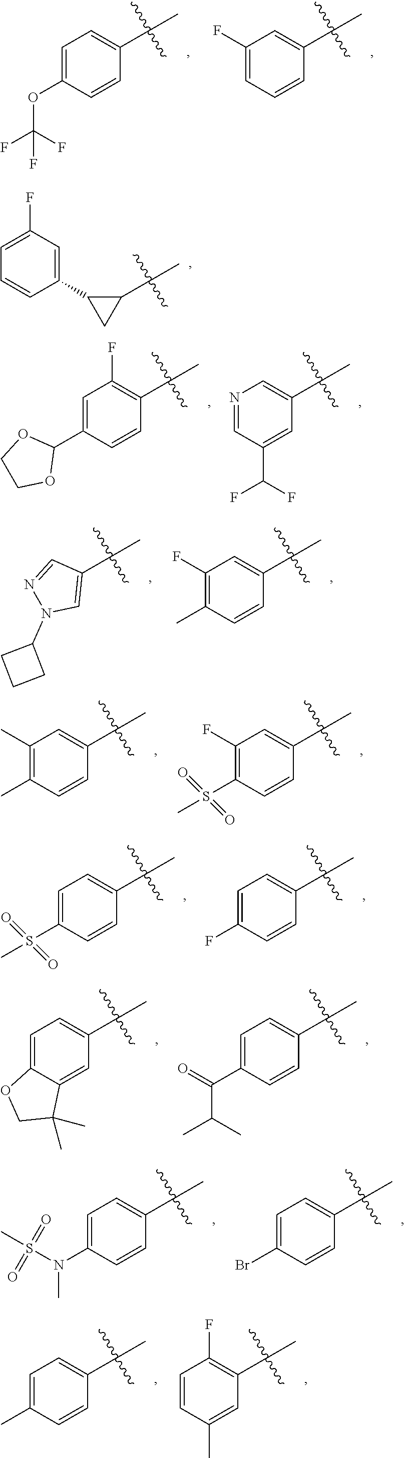

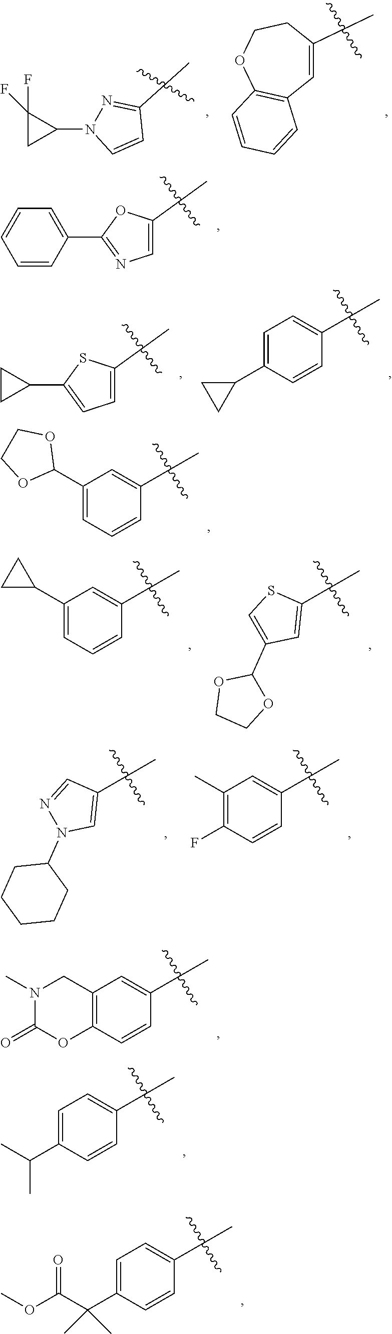

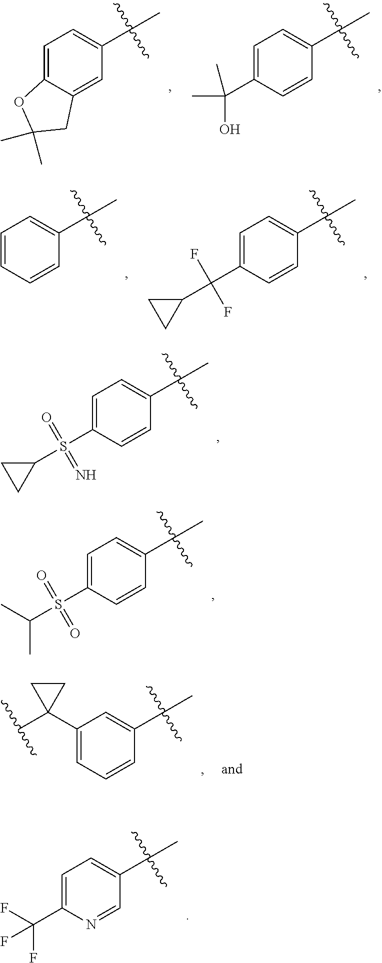

Embodiment 28. The compound of any one of Embodiments 1-27, wherein m is 0.
Embodiment 29. The compound of any one of Embodiments 1-27, wherein m is 1 or 2.
Embodiment 30. The compound of any one of Embodiments 1-27 or 29, wherein each R² is halo, —C(O)O—R² or an optionally substituted group selected from C₁-C₆ aliphatic and C₅-C₁₂ aryl.
Embodiment 31. The compound of any one of Embodiments 1-27 or 29, wherein R² is halo, —C(O)O—R² or optionally substituted C₁-C₆ aliphatic.
Embodiment 32. The compound of Embodiment 1, wherein R² is selected from F, Br, C₁, CN, —OCH₃, —CH₂—CF₃, —CF₃, —NH₂, —CH₂—O—CH₃, —CH₂—O—CH₂CH₃, —CH₂—CF₂—CH₃, —CH₃, —OH, oxo, S(O)₂,

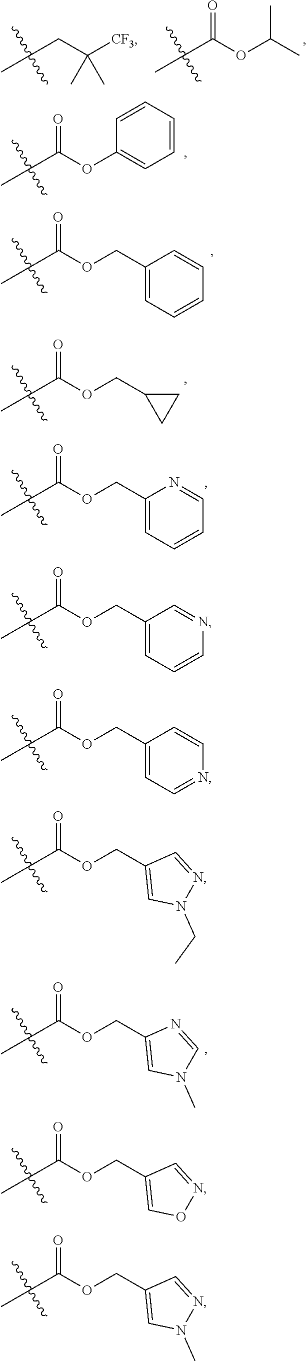

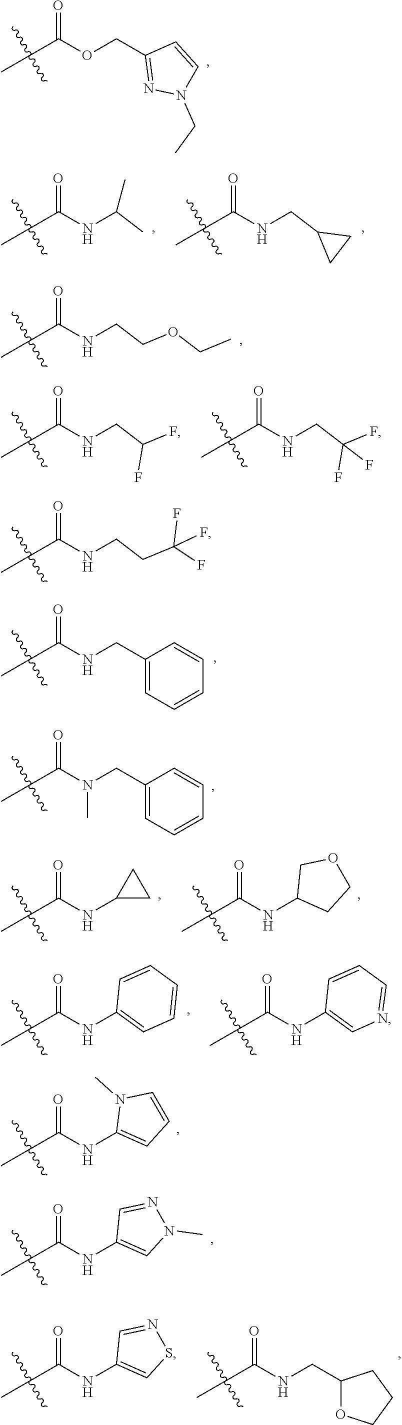

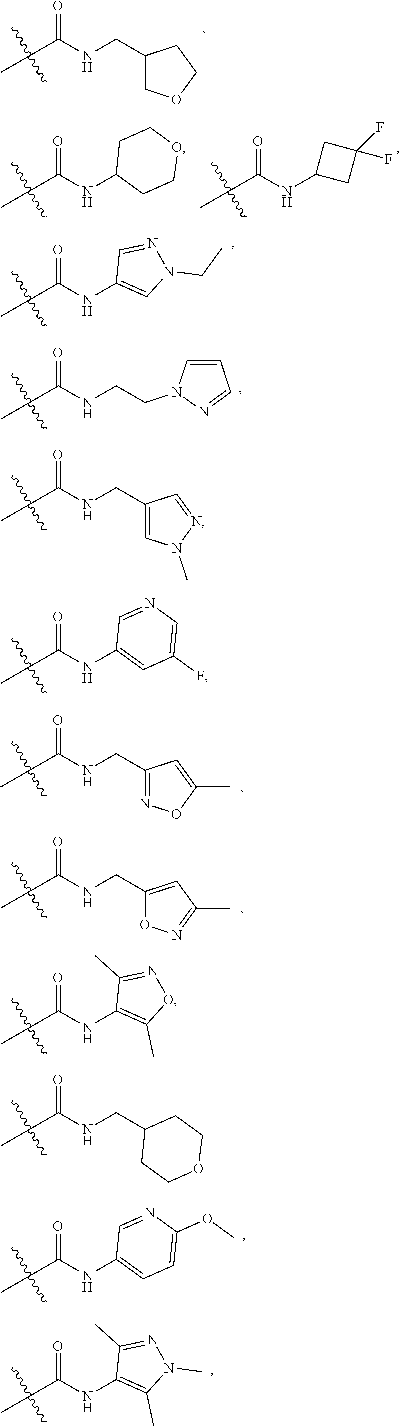

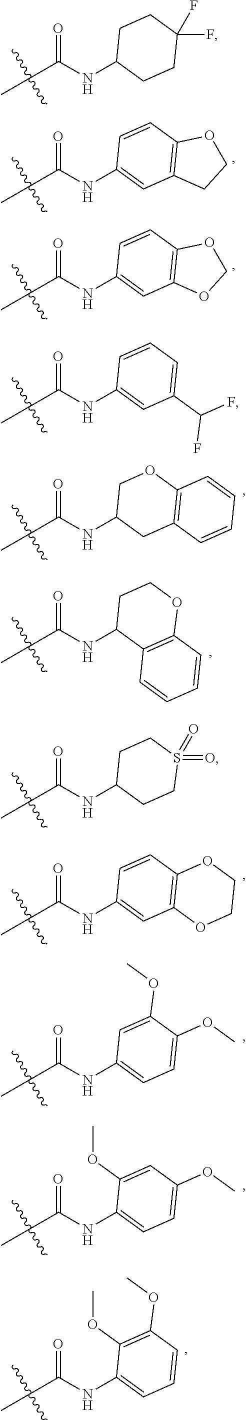

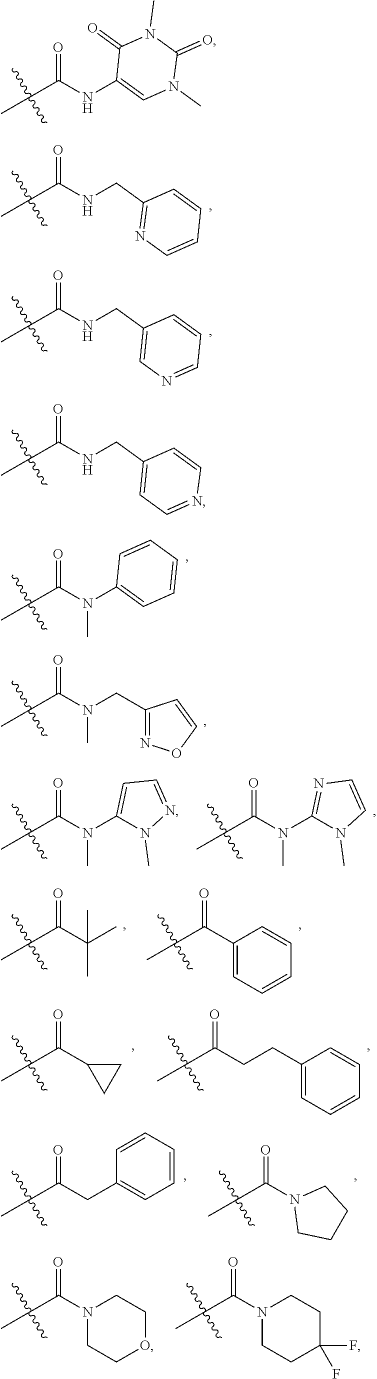

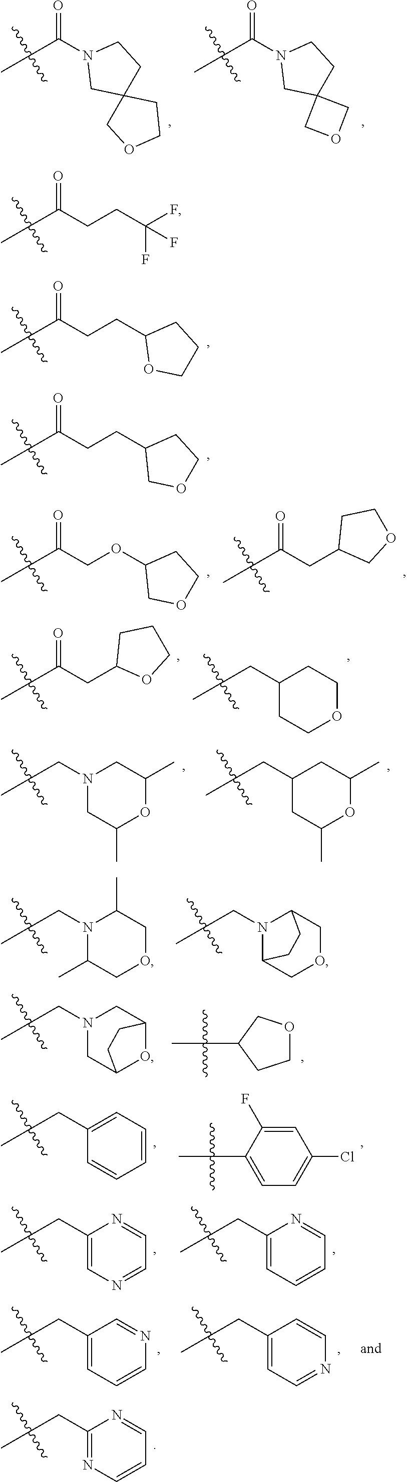

Embodiment 33. The compound of Embodiment 1, wherein the compound is of formula II:

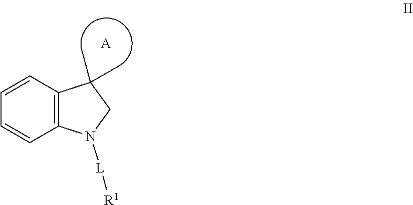

or a pharmaceutically acceptable salt thereof.
Embodiment 34. The compound of Embodiment 1, wherein the compound is of formula IIIa:

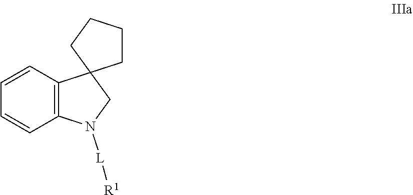

or a pharmaceutically acceptable salt thereof.
Embodiment 35. The compound of Embodiment 1, wherein the compound is of formula IIIb:

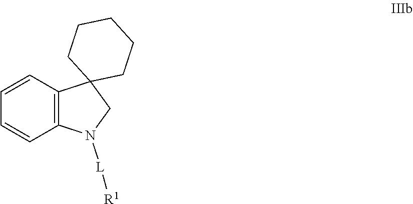

or a pharmaceutically acceptable salt thereof.
Embodiment 36. A compound of formula Ia:

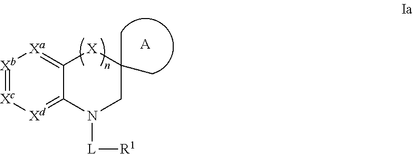

or a pharmaceutically acceptable salt thereof, wherein
   X is —NR⁵—, —C(R⁵)₂—, —C(O)—, or —O—;
   each of X^{a}, X^{b}, X, and X^{d} are independently selected from N and CR⁶;
   L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
   A is C₃-C₁₂ cycloaliphatic or 3- to 12-membered heterocyclyl comprising 1 to 3 heteroatoms selected from N, O, and S, wherein A is substituted with (R²)ₘ; R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
   each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
   each R^{2a} and each R^{2b} are independently selected from H and an optionally substituted group selected from C₁-C₆ aliphatic, C₃-C₁₂cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
   each R³ is independently halo, —S(O)₂—NR^{3a}R^{3b}, —S(O)₂—R^{3b}, —S(NR^{3c})(O)—NR^{3a}R^{3b}_, —S(O)(NR^{3c})—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
   R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
   each R^{3c} is independently selected from H, —OH, and optionally substituted C₁-C₆ aliphatic;
   each R⁵ is independently selected from hydrogen, halo, —CN, and optionally substituted C₁-C₆ aliphatic;
   each R⁶ is H, halo, CN, O—C₁-C₆ aliphatic, or an optionally substituted C₁-C₆ aliphatic;
   R⁸ is selected from H and optionally substituted C₁-C₆ aliphatic;
   n is 0 or 1,
   m is 0 to 4; and
   p is 0 to 4.
Embodiment 37. A compound of formula Ib

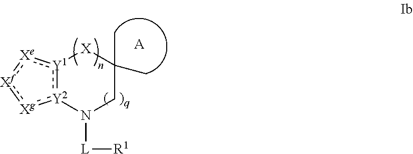

or a pharmaceutically acceptable salt thereof, wherein
   X is —NR⁵—, —C(R⁵)₂—, —C(O)—, or —O—;
   each of X^{e}, X^{f}, and X^{g} is independently selected from S, N, O, and CR⁷;
   each of Y¹ and Y² is independently selected from N and C;
   L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
   A is C₃-C₁₂ cycloaliphatic or 3- to 12-membered heterocyclyl comprising 1 to 3 heteroatoms selected from N, O, and S, wherein A is substituted with (R²)ₘ;
   R¹ is selected from C₁-C₆ aliphatic, C₃-C₁₂ cycloaliphatic, C₅-C₁₂ aryl, 5- to 12-membered heteroaryl comprising 1 to 3 heteroatoms selected from N, O, and S, and 3- to 12-membered heterocyclyl comprising 1 to 3 heteroatoms selected from N, O, and S, wherein R¹ is substituted with (R³)ₚ;
   each R² is independently halo, oxo, —NR^{2a}R^{2b}, —C(O)O—R^{2a}, —O—C(O)R^{2a}, —S(O)₂, —S(O)₂—R^{2a}, —C(O)—NR^{2a}R^{2b}, —N(R^{2a})—C(O)—R^{2b}, —C(O)—R^{2a}, —O—R^{2a}, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)—NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
   each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 3 heteroatoms selected from N, O, and S;
   each R³ is independently halo, —S(O)₂—NR^{3a}R^{3b}, —S(O)₂—R^{3b}, —S(NR^{3c})(O)—NR^{3a}R^{3b}, —S(O)(NR^{3c})—R^{3b}, —S(O)—R^{3b}, —NR^{3a}—S(O)₂—R^{3b}, —O—R^{3a}, —C(O)—R^{3a}, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
   R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
   each R^{3c} is independently selected from H, —OH, and optionally substituted C₁-C₆ aliphatic;
   each R⁵ is independently selected from hydrogen, halo, —CN, and optionally substituted C₁-C₆ aliphatic;
   each R⁷ is H, halo, CN, O—C₁-C₆ aliphatic, or an optionally substituted group selected from C₁-C₆ aliphatic and C₃-C₆ cycloaliphatic;
   R⁸ is selected from H and optionally substituted C₁-C₆ aliphatic;
   n is 0 or 1;
   m is 0 to 4; and
   p is 0 to 4.
Embodiment 38. A compound selected from Table 1.
Embodiment 39. A compound selected from Table 2.
Embodiment 40. A pharmaceutical composition comprising a compound of any one of Embodiments 1-39 and a pharmaceutically acceptable carrier or excipient.
Embodiment 41. A method of modulating a TRPML (e.g., TRPML1, TRPML2, TRPML3, or combinations thereof) comprising administering to a subject a compound of any one Embodiments 1-39, or a composition thereof.
Embodiment 42. A method of treating a disease, disorder, or condition in a subject comprising administering a compound of any one Embodiments 1-39, or a composition thereof.
Embodiment 43. The method of Embodiment 42, wherein the disease, disorder, or condition is associated with TPRML (e.g., TRPML1, TRPML2, TRPML3, or combinations thereof) modulation.
Embodiment 44. The method of Embodiments 42 or 43, wherein the disease, disorder, or condition is a lysosomal storage disorder.
Embodiment 45. The method of Embodiment 44, wherein the lysosomal storage disorder is selected from Niemann-Pick C disease, Gaucher disease, and Pompe disease.
Embodiment 46. The method of Embodiments 42 or 43, wherein the disease, disorder, or condition is age-related common neurodegenerative disease.
Embodiment 47. The method of Embodiment 46, wherein the disease, disorder, or condition is selected from Alzheimer's Disease, Parkinson's Disease, and Huntington's Disease.
Embodiment 48. The method of Embodiment 43, wherein the disease, disorder, or condition is a type IV Mucolipidosis (ML4) neurodegenerative lysosomal storage disease caused by mutations in TRPML (e.g., TRPML1, TRPML2, and/or TRPML3).
Embodiment 49. The method of Embodiment 42, wherein the disease, disorder, or condition is a muscular disease, a liver disease, a metabolic disease, an atherosclerotic disease, an inflammatory bowel disease, an atherosclerotic disease, a neurodegenerative disease, an oncological disease, or an infectious disease.
Embodiment 50. The method of Embodiment 49, wherein the disease, disorder, or condition is a muscular disease.
Embodiment 51. The method of Embodiment 50, wherein the muscular disease is a muscular dystrophy.
Embodiment 52. The method of Embodiment 51, wherein the muscular dystrophy is Duchenne muscular dystrophy.
Embodiment 53. The method of Embodiment 49, wherein the disease, disorder, or condition is an infectious disease.
Embodiment 54. The method of Embodiment 53, wherein the infectious disease is an infection of Heliobaccer pylori or Mycobacterium tuberculosis.

### EXEMPLIFICATION

The present teachings include descriptions provided in the Examples that are not intended to limit the scope of any claim. Unless specifically presented in the past tense, inclusion in the Examples is not intended to imply that the experiments were actually performed. The following non-limiting examples are provided to further illustrate the present teachings. Those of skill in the art, in light of the present application, will appreciate that many changes can be made in the specific embodiments that are provided herein and still obtain a like or similar result without departing from the spirit and scope of the present teachings.

Table of Abbreviations

| | |
|---|---|
| ACN | Acetonitrile |
| aq. | aqueous |
| Boc | tert-Butyloxycarbonyl |
| Brettphos | 2-(Dicyclohexylphosphino)3,6-dimethoxy-2′,4′,6′-triisopropyl- |
| | 1,1′-biphenyl |
| CDI | 1,1′-Carbonyldiimidazole |
| DAST | Diethylaminosulfur trifluoride |
| DCM | Dichloromethane |
| DIBAL | Diisobutylaluminum hydride |
| DIPEA | N,N-Diisopropylethylamine |
| DMAP | 4-dimethylaminopyridine |
| DMSO | Dimethylsulfoxide |
| EDC | 1-Ethyl-3-(3-dimethylaminopropyl)carbodiimide |
| eq | Equivalent |
| h | Hour or hours |
| HOBT | 1-Hydroxy-7-azabenzotriazole |
| HPLC | High pressure liquid chromatography |
| LCMS | Liquid chromatography mass spectrometry |
| LDA | Lithium diisopropylamide |
| LiHMDS | Lithium bis(trimethylsilyl)amide |
| mCPBA | meta-Chloroperoxybenzoic acid |
| MOST | Morpholinosulfur trifluoride |
| MTBE | Methyl tert-butyl ether |
| MW | Microwave |
| NMP | N-Methyl-2-pyrrolidone |
| NMR | Nuclear Magnetic Resonance |
| Pd₂(dba)₃ | Tris(dibenzylideneacetone)dipalladium(0) |
| Py | Pyridine |
| RT | Room temperature |
| TBAF | Tetrabutylammonium fluoride |
| TFA | Trifluoroacetic acid |
| THF | Tetrahydrofuran |
| TLC | Thin layer chromatography |

### Analytical Instrumentation and Purification:

NMR Instrument Details: Varian 400 MHz, Probe-1: Auto XID Probe 2: ATB.

LCMS Instrument Details: Shimadzu LCMS-2010EV system coupled to SPD-M20A PDA and ELS detectors. Softa model 400.

### LCMS Method 1—Acidic Conditions

Column: X-Select C18 CSH (3.0*50) mm 2.5μ; Make: Waters
Mobile Phase A: 0.05% formic acid in water: Acetonitrile (95:5); pH=3.5
Mobile Phase B: 0.05% formic acid in Acetonitrile
Column oven temperature: 50 C
Flow rate: 1.2 ml/minute
PDA: 210 nm Maxplot
Gradient program:

| Time(min) | A % | B % |
|---|---|---|
| 0.0 | 100 | 0 |
| 2.0 | 2 | 98 |
| 3.0 | 2 | 98 |
| 3.2 | 100 | 0 |
| 4.0 | 100 | 0 |

MS Parameters
Mode: Dual (+/−)
Detector voltage: 1.5 KV
Scan rang: 80-2000 amu
Scan speed: 2000

### LCMS Method 2—Basic Conditions

Column: X-Select C18 CSH (3.0*50) mm 2.5 μm; Make: Waters
Mobile Phase A: 5 mM Ammonium Bicarb; pH=8.8
Mobile Phase B: Acetonitrile
Column oven temperature: 50 C
Flow rate: 1.2 ml/minute
PDA: 210 nm Maxplot
Gradient program:

| Time(min) | A % | B % |
|---|---|---|
| 0.0 | 100 | 0 |
| 2.0 | 2 | 98 |
| 3.0 | 2 | 98 |
| 3.2 | 100 | 0 |
| 4.0 | 100 | 0 |

MS Parameters
Mode: Dual (+/−)
Detector voltage: 1.5 KV
Scan rang: 80-2000 amu
Scan speed: 2000

### HPLC Method 1—Acidic Conditions

Column: X-Select CSH C18 (4.6*150) mm; 5ρ; Make: Waters
Mobile Phase: A—0.1% Formic acid in water: Acetonitrile (95:05); pH=3.5
B—Acetonitrile
Flow Rate: 1.0. mL/minute
PDA: 210 nm maxplot
Gradient program:

| Time(min | A % | B % |
|---|---|---|
| 0.0 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### HPLC Method 2—Basic Conditions

Column: Xbridge C18 (4.6*150) mm, 5μ: Make: Waters
Mobile Phase A—0.1% NH3 in water; pH=9.5
B—Acetonitrile
Flow Rate: 1.2. mL/minute
PDA: 210 nm maxplot
Gradient program:

| Time(min) | A % | B % |
|---|---|---|
| 0.0 | 98 | 2 |
| 6.0 | 0 | 85 |
| 8.0 | 0 | 85 |
| 9.0 | 0 | 100 |
| 12.0 | 0 | 100 |
| 14.0 | 98 | 2 |
| 18.0 | 98 | 2 |

### SYNTHETIC EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Synthesis of Certain Intermediates

### Spiro[cyclopentane-1,3′-indoline] (Compound 1.4)

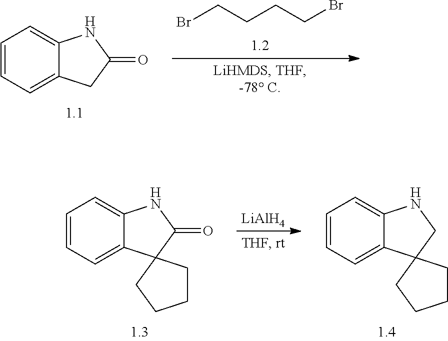

### Step-1: Procedure for Synthesis of spiro[cyclopentane-1,3′-indolin]-2′-one 3

To a solution of indolin-2-one (1.1, 20 g, 150 mmol) in THF (200 mL) cooled to −78° C., was added LiHMDS (1.0 M in THF, 300 mL, 300 mmol) dropwise. It was slowly warmed to −50° C. and stirred for 30 min, followed by cooling to −78° C. and 1,5-dibromobutane (1.2, 35.7 g, 165 mmol) was added. The reaction mixture was stirred at room temperature for 3 h. The progress of the reaction was monitored by thin layer chromatography (TLC). After completion of reaction as monitored by TLC, the reaction mixture was quenched with saturated solution of ammonium chloride and extracted with ethyl acetate. The combined organic layers were washed with water, brine, dried over anhydrous sodium sulfate and concentrated under reduced pressure to dryness to afford spiro[cyclopentane-1,3′-indolin]-2′-one 3 (1.3, 16 g, crude). This compound was used in the next step without further purification. LCMS: 188.0 [M+H]+.

### Step-2: Procedure for Synthesis of spiro[cyclopentane-1,3′-indoline 4

To a solution of spiro[cyclopentane-1,3′-indolin]-2′-one 3 (1.3, 16 g, 85.5 mmol) in THF (200 mL) cooled at 0° C. was added LiAlH4 (1.0 M in THF, 111 mL, 111.2 mmol) dropwise. The reaction mixture was stirred at room temperature for 4 h and refluxed at 80° C. for 2 hours, following which it was cooled to room temperature and quenched with saturated aq. Na₂SO₄ solution. The resulting slurry was filtered through a pad of Celite, the filtrate was washed with ethyl acetate and evaporated under reduced pressure. The crude product was purified by silica gel column chromatography to afford spiro[cyclopentane-1,3′-indoline 4 (1.3, 10 g, 71%) as a yellowish powder. LCMS: 174.10 [M+H]+.

### Generic Synthesis of Certain Compounds

Method 1

In some embodiments, compounds described herein are prepared according to the following scheme:

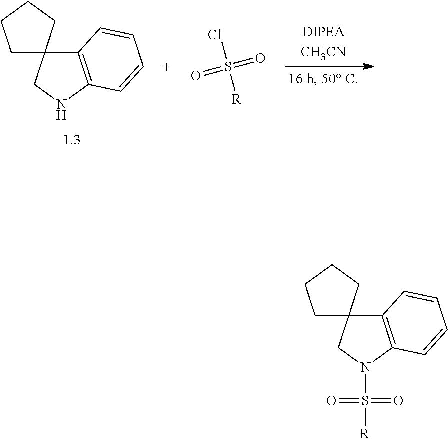

Spiro[cyclopentane-1,3′-indoline] (1.3, 1 eq.) in dry acetonitrile (0.7 mL) and diisopropylethylamine (1.5 eq.) were placed in vial and sulfonyl chloride (1.1 eq.) was added. The reaction mixture was stirred for 16 hours at 50° C. After cooling to room temperature the mixture was evaporated. The residue was dissolved in DMSO, filtered, and the solution was subjected to HPLC purification (deionized water/HPLC-grade methanol (acetonitrile)).

Method 2

In some embodiments, compounds described herein are prepared according to the following scheme:

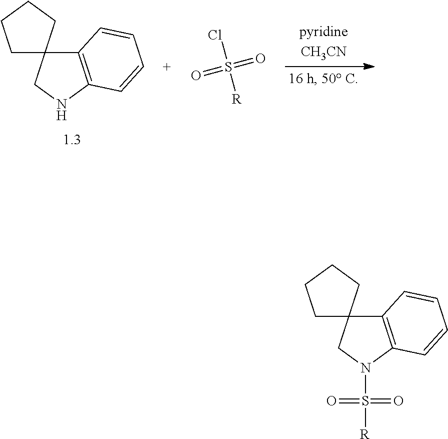

To a stirred solution of spiro[cyclopentane-1,3′-indoline 1.3 (100 mg, 0.57 mmol, 1 eq) in acetonitrile (10 mL) was added pyridine (0.09 mL, 1.1 mmol, 2 eq) at room temperature and stirred for 5 min, sulfonyl chloride (1 eq) was added and the reaction mixture was stirred at room temperature for 2 h. The progress of the reaction was monitored by TLC. After completion of the reaction, the reaction mixture was diluted with water and extracted with ethyl acetate. The combined organic layers were dried over anhydrous Na₂SO₄, filtered and concentrated under reduced pressure. The crude product was purified by reverse phase preparative HPLC.

### Example 1—Compounds Prepared According to Method 1

The following compounds were prepared according to Method 1. A person of skill in the art would understand what sulfonyl chloride compounds having certain values for variable R would be used in the generic scheme to prepare the compounds provided below, using standard chemical manipulations and procedures similar to those used for the preparation of Method 1.

| Compound | | |
|---|---|---|
| No. | Structure | Analytical data |
| I-1 | 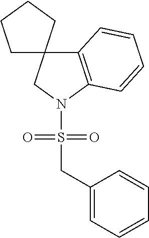 [SMILES: O=S(=O)(Cc1ccccc1)N1CC2(CCCC2)c2ccccc21] | Yield: 19.6 mg, 12.3%; Appearance: Beige solid; ¹H NMR (600 MHz, DMSO-d₆) δ 7.28 (ddt, J = 15.5, 7.8, 2.8 Hz, 5H), 7.21 (d, J = 7.5 Hz, 1H), 7.11 (d, J = 3.9 Hz, 2H), 6.98 (dq, J = 8.0, 4.3 Hz, 1H), 4.57 (s, 2H), 3.57 (s, 2H), 1.87 - 1.68 (m, 6H), 1.68 1.48 (m, 2H); HPLC purity: 98.07%; LCMS Calculated for C₁₉H₂₁NO₂S: 327.44; Observed: 327.2 [M + H]⁺. |
| I-2 | 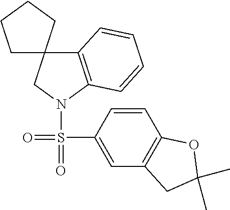 [SMILES: CC1(C)Cc2cc(S(=O)(=O)N3CC4(CCCC4)c4ccccc43)ccc2O1] | Yield: 57.0 mg, 31.4%; Appearance: Yellow solid; ¹H NMR (400 MHz, DMSO-d₆) δ 7.58 (d, J = 1.9 Hz, 1H), 7.53 (dd, J = 8.4, 2.0 Hz, 1H), 7.47 (d, J = 8.0 Hz, 1H), 7.15 (td, J = 7.8, 1.4 Hz, 1H), 7.05 (dd, J = 7.5, 1.4 Hz, 1H), 7.01 - 6.90 (m, 1H), 6.71 (d, J = 8.4 Hz, 1H), 3.60 (s, 2H), 3.03 (s, 2H), 1.86 - 1.52 (m, 8H), 1.44 (s, 6H); HPLC purity: 100%; LCMS Calculated for C₂₂H₂₅NO₃S: 383.51; Observed: 384.2 [M + H]⁺. |
| I-3 | 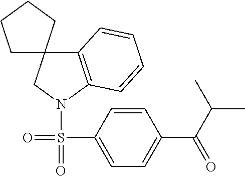 [SMILES: CC(C)C(=O)c1ccc(S(=O)(=O)N2CC3(CCCC3)c3ccccc32)cc1] | Yield: 78.2 mg, 45.2%; Appearance: Beige solid; ¹H NMR (600 MHz, DMSO-d₆) δ 8.11 - 8.02 (m, 2H), 7.96 - 7.87 (m, 2H), 7.47 (d, J = 8.1 Hz, 1H), 7.20 (ddd, J = 8.2, 7.5, 1.3 Hz, 1H), 7.14 (dd, J = 7.5, 1.3 Hz, 1H), 7.01 (td, J = 7.5, 1.1 Hz, 1H), 3.71 (s, 2H), 3.59 (p, J = 6.8 Hz, 1H), 1.68 (ddq, J = 9.8, 6.1, 2.9 Hz, 2H), 1.66 - 1.56 (m, 2H), 1.53 (dddd, J = 12.1, 7.5, 5.4, 2.8 Hz, 2H), 1.49 - 1.35 (m, 2H), 1.03 (dd, J = 69, 1.1 Hz, 6H); HPLC purity: 97.60%; LCMS Calculated for C₂₂H₂₅NO₃S: 383.51; Observed: 383.51 [M + H]⁺. |
| I-4 | 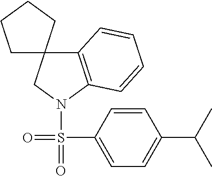 [SMILES: CC(C)c1ccc(S(=O)(=O)N2CC3(CCCC3)c3ccccc32)cc1] | Yield: 83.8 mg, 48.8%; Appearance: Grey solid; ¹H NMR (400 MHz, Chloroform-d) δ 7.75 (d, J = 8.1 Hz, 2H), 7.66 (d, J = 8.1 Hz, 1H), 7.30 (d, J = 8.0 Hz, 2H), 7.25 - 7.15 (m, 1H), 7.04 (dt, J = 14.7, 7.3 Hz, 2H), 3.69 (s, 2H), 2.94 (p, J = 6.9 Hz, 1H), 1.79 (s, 2H), 1.73 - 1.61 (m, 4H), 1.57 (s, 2H), 1.23 (d, J = 6.9 Hz, 6H); HPLC purity: 100%; LCMS Calculated for C₂₁H₂₅NO₂S: 355.50; Observed: 356.2 [M + H]⁺. |
| I-5 | 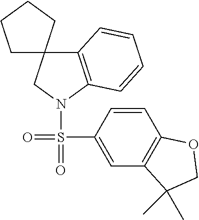 [SMILES: CC1(C)COc2ccc(S(=O)(=O)N3CC4(CCCC4)c4ccccc43)cc21] | Yield: 28.4 mg, 17.6%; Appearance: Yellow solid; ¹H NMR (600 MHz, DMSO-d₆) δ 7.61 (d, J = 2.0 Hz, 1H), 7.54 - 7.48 (m, 1H), 7.48 - 7.43 (m, 1H), 7.18 (ddt, J = 8.4, 7.6, 1.3 Hz, 1H), 7.11 (dd, J = 7.4, 1.4 Hz, 1H), 6.99 (td, J = 7.5, 1.1 Hz, 1H), 6.85 (dd, J = 8.4, 1.4 Hz, 1H), 4.27 (d, J = 1.4 Hz, 2H), 3.66 (d, J = 1.4 Hz, 2H), 1.67 (ddq, J = 9.9, 6.2, 3.1 Hz, 2H), 1.65 - 1.57 (m, 2H), 1.54 - 1.43 (m, 2H), 1.41 - 1.31 (m, 2H), 1.21 (d, J = 1.5 Hz, 6H); HPLC purity: 100%; LCMS Calculated for C₂₂H₂₅NO₃S: 383.51; Observed: 384.4 [M + H]⁺. |
| I-6 | 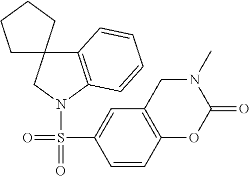 [SMILES: CN1Cc2cc(S(=O)(=O)N3CC4(CCCC4)c4ccccc43)ccc2OC1=O] | Yield: 62.8 mg, 36.1%; Appearance: Pink solid; ¹H NMR (400 MHz, DMSO-d₆) δ 7.85 - 7.75 (m, 1H), 7.69 (dd, J = 8.4, 2.2 Hz, 1H), 7.46 (d, J = 8.1 Hz, 1H), 7.16 (t, J = 7.5 Hz, 1H), 7.12 - 7.02 (m, 2H), 6.97 (t, J = 7.4 Hz, 1H), 4.53 (s, 2H), 3.65 (s, 2H), 3.01 (s, 3H), 1.91 - 1.51 (m, 8H); HPLC purity: 100%; LCMS Calculated for C₂₁H₂₂N₂O₄S: 398.48; Observed: 399.2 [M + H]⁺. |
| I-7 | 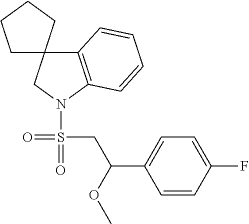 [SMILES: COC(CS(=O)(=O)N1CC2(CCCC2)c2ccccc21)c1ccc(F)cc1] | Yield: 48.9 mg, 27.4%; Appearance: Pink oil; ¹H NMR (600 MHz, DMSO-d₆) δ 7.39 - 7.30 (m, 2H), 7.26 (d, J = 8.0 Hz, 1H), 7.21 (dd, J = 7.5, 1.3 Hz, 1H), 7.18 - 7.11 (m, 3H), 6.98 (td, J = 7.4, 1.0 Hz, 1H), 4.64 (dd, J = 9.3, 3.0 Hz, 1H), 3.80 - 3.75 (m, 1H), 3.75 - 3.69 (m, 2H), 3.36 (dd, J = 14.8, 3.0 Hz, 1H), 2.84 (s, 3H), 2.00 - 1.54 (m, 8H); HPLC purity: 100%; LCMS Calculated for C₂₁H₂₄FNO₃S: 389.49; Observed: 389.49 [M + H]⁺. |
| I-8 | 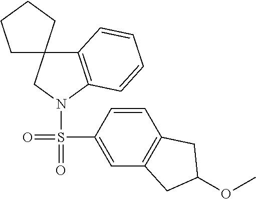 [SMILES: COC1Cc2ccc(S(=O)(=O)N3CC4(CCCC4)c4ccccc43)cc2C1] | Yield: 14.4 mg, 9.25%; Appearance: Yellow oil; ¹H NMR (600 MHz, DMSO-d₆) δ 7.66 (d, J = 1.7 Hz, 1H), 7.58 (dd, J = 7.9, 1.7 Hz, 1H), 7.42 (d, J = 8.1 Hz, 1H), 7.37 (d, J = 7.9 Hz, 1H), 7.16 (td, J = 7.8, 1.2 Hz, 1H), 7.12 (dd, J = 7.5, 1.2 Hz, 1H), 6.97 (t, J = 7.5 Hz, 1H), 4.15 (tt, J = 6.1, 3.1 Hz, 1H), 3.65 (s, 2H), 3.17 (s, 3H) , 3.09 (d, J = 6.1 Hz, 1H), 3.06 (d, J = 6.1 Hz, 1H), 2.90 (dd, J = 7.6, 3.1 Hz, 1H), 2.87 (dd, J = 8.0, 3.0 Hz, 1H), 1.69 (tt, J = 7.8, 4.0 Hz, 2H), 1.66 - 1.60 (m, 2H), 1.57 (dd, J = 12.8, 6.5 Hz, 2H), 1.51 (dq, J = 14.0, 8.4, 7.0 Hz, 2H); HPLC purity: 100%; LCMS Calculated for C₂₂H₂₅NO₃S: 383.51; Observed: 384.2 [M + H]⁺. |
| I-9 | 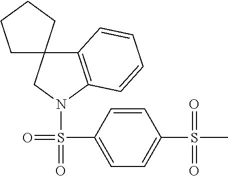 [SMILES: CS(=O)(=O)c1ccc(S(=O)(=O)N2CC3(CCCC3)c3ccccc32)cc1] | Yield: 45.8 mg, 25.4%; Appearance: Beige solid; ¹H NMR (600 MHz, DMSO-d₆) δ 8.09 (s, 4H), 7.46 (d, J = 8.1 Hz, 1H), 7.20 (t, J = 7.8 Hz, 1H), 7.16 (d, J = 7.4 Hz, 1H), 7.02 (t, J = 7.4 Hz, 1H), 3.74 (s, 2H), 3.25 (s, 3H), 1.69 (dq, J = 11.4, 4.4 Hz, 2H), 1.67 - 1.60 (m, 2H), 1.57 (dt, J = 14.1, 7.0 Hz, 2H), 1.54 - 1.40 (m, 2H); HPLC purity: 100%; LCMS Calculated for C₁₉H₂₁NO₄S₂: 391.50; Observed: 392.2 [M + H]⁺. |
| I-10 | 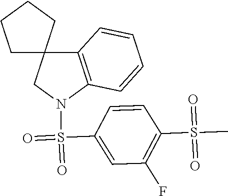 [SMILES: CS(=O)(=O)c1ccc(S(=O)(=O)N2CC3(CCCC3)c3ccccc32)cc1F] | Yield: 6.7 mg, 4.15%; Appearance: Light brown solid; ¹H NMR (600 MHz, DMSO-d₆) δ 8.12 - 7.99 (m, 2H), 7.91 (dd, J = 8.1, 1.7 Hz, 1H), 7.46 (d, J = 8.1 Hz, 1H), 7.26 - 7.13 (m, 2H), 7.04 (t, J = 7.5 Hz, 1H), 3.78 (s, 2H), 3.35 (s, 3H), 1.72 (p, J = 5.2, 4.3 Hz, 2H), 1.70 - 1.63 (m, 2H), 1.63 - 1.58 (m, 2H), 1.56 (dq, J = 13.4, 5.3 Hz, 2H); HPLC purity: 100%; LCMS Calculated for C₁₉H₂₀FNO₄S₂: 409.49; Observed: 406.0 [M − 3H]⁻. |
| I-11 | 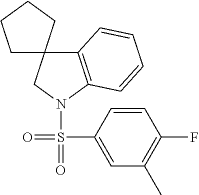 [SMILES: Cc1cc(S(=O)(=O)N2CC3(CCCC3)c3ccccc32)ccc1F] | Yield: 64.9 mg, 36.6%; Appearance: Yellow solid; ¹H NMR (400 MHz, DMSO-d₆) δ 7.74 (dd, J = 7.2, 2.3 Hz, 1H), 7.65 (ddd, J = 8.1, 4.8, 2.5 Hz, 1H), 7.49 (d, J = 8.0 Hz, 1H), 7.18 (q, J = 8.4, 7.5 Hz, 2H), 7.06 (d, J = 8.0 Hz, 1H), 6.98 (t, J = 7.4 Hz, 1H), 3.64 (s, 2H), 2.30 (d, J = 2.1 Hz, 3H), 1.92 - 1.45 (m, 8H); HPLC purity: 100%; LCMS Calculated for C₁₉H₂₀FNO₂S: 345.43; Observed: 347.0 [M + 2H]⁺. |
| I-12 | 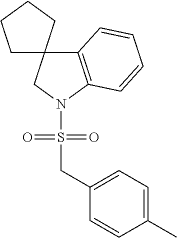 [SMILES: Cc1ccc(CS(=O)(=O)N2CC3(CCCC3)c3ccccc32)cc1] | Yield: 29.5 mg, 16.6%; Appearance: Pink solid; ¹H NMR (400 MHz, Chloroform-d) δ 7.38 - 7.25 (m, 2H), 7.20 (t, J = 7.6 Hz, 1H), 7.13 (s, 4H), 7.04 (t, J = 7.4 Hz, 1H), 4.37 (d, J = 2.9 Hz, 2H), 3.47 (s, 2H), 2.34 (d, J = 2.9 Hz, 3H), 1.76 (s, 4H), 1.73 - 1.46 (m, 4H); HPLC purity: 98.75%; LCMS Calculated for C₂₀H₂₃NO₂S: 341.47; Observed: 341.47 [M + H]⁺. |
| I-13 | 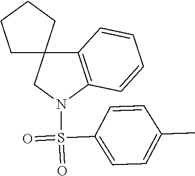 [SMILES: Cc1ccc(S(=O)(=O)N2CC3(CCCC3)c3ccccc32)cc1] | Yield: 3.2 mg, 1.75%; Appearance: Yellow solid; ¹H NMR (600 MHz, DMSO-d₆) δ 7.68 (d, J = 7.9 Hz, 2H), 7.44 (d, J = 8.1 Hz, 1H), 7.34 (d, J = 7.9 Hz, 2H), 7.17 (t, J = 7.8 Hz, 1H), 7.12 (d, J = 7.5 Hz, 1H), 6.98 (t, J = 7.5 Hz, 1H), 3.65 (d, J = 1.4 Hz, 2H), 2.31 (s, 3H), 1.69 (q, J = 7.0, 6.0 Hz, 2H), 1.67 - 1.58 (m, 2H), 1.55 (dt, J = 14.4, 7.0 Hz, 2H), 1.51 - 1.40 (m, 2H); HPLC purity: 100%; LCMS Calculated for C₁₉H₂₁NO₂S: 327.44; Observed: 328.2 [M + H]⁺. |
| I-14 | 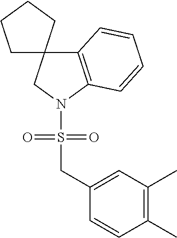 [SMILES: Cc1ccc(CS(=O)(=O)N2CC3(CCCC3)c3ccccc32)cc1C] | Yield: 21.0 mg, 11.7%; Appearance: Pink solid; ¹H NMR (600 MHz, DMSO-d₆) δ 7.20 (dt, J = 7.4, 1.0 Hz, 1H), 7.14 - 7.09 (m, 2H), 7.05 (d, J = 7.7 Hz, 1H), 6.98 (dddd, J = 8.5, 5.7, 2.7, 1.3 Hz, 2H), 6.94 (d, J = 1.8 Hz, 1H), 4.46 (s, 2H), 3.56 (s, 2H), 2.15 (s, 3H), 2.08 (s, 3H), 1.84 - 1.56 (m, 8H); HPLC purity: 100%; LCMS Calculated for C₂₁H₂₅NO₂S: 355.50; Observed: 355.50 [M − H]⁻. |
| I-15 | 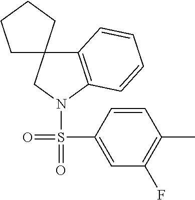 [SMILES: Cc1ccc(S(=O)(=O)N2CC3(CCCC3)c3ccccc32)cc1F] | Yield: 21.2 mg, 11.9%; Appearance: Brown solid; ¹H NMR (600 MHz, DMSO-d₆) δ 7.60 - 7.53 (m, 2H), 7.48 (t, J = 7.7 Hz, 1H), 7.44 (d, J = 8.1 Hz, 1H), 7.24 - 7.17 (m, 1H), 7.14 (dd, J = 7.6, 1.3 Hz, 1H), 7.00 (td, J = 7.5, 1.1 Hz, 1H), 3.69 (s, 2H), 2.23 (d, J = 1.8 Hz, 3H), 1.70 (dtt, J = 11.3, 8.2, 4.9 Hz, 2H), 1.64 (dt, J = 12.4, 8.0 Hz, 2H), 1.57 (dt, J = 14.0, 7.0 Hz, 2H), 1.54 - 1.45 (m, 2H); HPLC purity: 98.74%; LCMS Calculated for C₁₉H₂₀FNO₂S: 345.43; Observed: 345.43 [M + H]⁺. |
| I-16 | 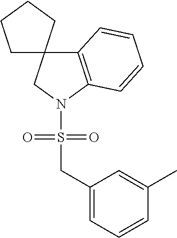 [SMILES: Cc1cccc(CS(=O)(=O)N2CC3(CCCC3)c3ccccc32)c1] | Yield: 21.4 mg, 13.6%; Appearance: Beige solid; ¹H NMR (600 MHz, DMSO-d₆) δ 7.21 (dd, J = 7.5, 1.0 Hz, 1H), 7.18 (t, J = 7.6 Hz, 1H), 7.14 - 7.10 (m, 3H), 7.07 (d, J = 7.6 Hz, 1H), 7.00 (s, 1H), 6.99 - 6.95 (m, 1H), 4.53 (s, 2H), 3.56 (s, 2H), 2.18 (s, 3H), 1.83 - 1.57 (m, 8H); HPLC purity: 95.85%; LCMS Calculated for C₂₀H₂₃NO₂S: 341.47; Observed: 341.47 [M + H]⁺. |
| I-17 | 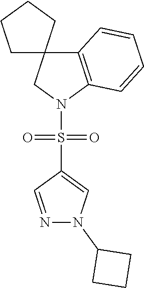 [SMILES: O=S(=O)(c1cnn(C2CCC2)c1)N1CC2(CCCC2)c2ccccc21] | Yield: 48.7 mg, 27.0%; Appearance: Beige solid; ¹H NMR (600 MHz, DMSO-d₆) δ 8.52 (s, 1H), 7.80 (d, J = 1.4 Hz, 1H), 7.38 (d, J = 8.0 Hz, 1H), 7.21 - 7.11 (m, 2H), 6.99 (t, J = 7.5 Hz, 1H), 4.81 (p, J = 8.4 Hz, 1H), 3.61 (d, J = 1.4 Hz, 2H), 2.36 (pd, J = 10.4, 9.8, 2.0 Hz, 2H), 2.32 - 2.24 (m, 2H), 1.81 - 1.43 (m, 10H); HPLC purity: 100%; LCMS Calculated for C₁₉H₂₃N₃O₂S: 357.47; Observed: 358.2 [M + H]⁺. |
| I-18 | 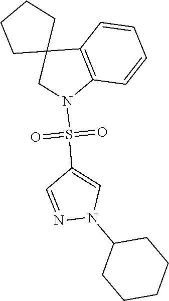 [SMILES: O=S(=O)(c1cnn(C2CCCCC2)c1)N1CC2(CCCC2)c2ccccc21] | Yield: 76.8 mg, 42.8%; Appearance: Brown oil; ¹H NMR (400 MHz, DMSO-d₆) δ 8.26 (s, 1H), 7.54 (s, 1H), 7.43 (d, J = 8.1 Hz, 1H), 7.22 - 7.12 (m, 1H), 7.07 (d, J = 7.5 Hz, 1H), 6.98 (t, J = 7.5 Hz, 1H), 4.20 - 3.99 (m, 1H), 3.59 (s, 2H), 1.98 (d, J = 11.9 Hz, 2H), 1.83 (d, J = 16.4 Hz, 5H), 1.77 - 1.54 (m, 8H), 1.39 (q, J = 13.2 Hz, 2H), 1.23 (q, J = 12.7, 12.2 Hz, 1H); HPLC purity: 97.87%; LCMS Calculated for C₂₁H₂₇N₃O₂S: 385.53; Observed: 386.2 [M + H]⁺. |
| I-19 | 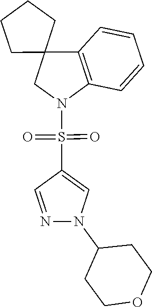 [SMILES: O=S(=O)(c1cnn(C2CCOCC2)c1)N1CC2(CCCC2)c2ccccc21] | Yield: 73.5 mg, 42.0%; Appearance: Yellow solid; ¹H NMR (400 MHz, DMSO-d₆) δ 8.37 (s, 1H), 7.59 (s, 1H), 7.44 (d, J = 8.1 Hz, 1H), 7.23 - 7.12 (m, 1H), 7.08 (d, J = 8.0 Hz, 1H), 6.98 (t, J = 7.4 Hz, 1H), 4.39 (ddd, J = 15.5, 10.0, 5.9 Hz, 1H), 3.94 (dt, J = 11.8, 3.2 Hz, 2H), 3.60 (s, 2H), 3.43 (td, J = 11.4, 3.7 Hz, 2H), 1.95 (td, J = 9.7, 8.8, 3.7 Hz, 4H), 1.88 - 1.53 (m, 8H); HPLC purity: 100%; LCMS Calculated for C₂₀H₂₅N₃O₃S: 387.50; Observed: 388.2 [M + H]⁺. |
| I-20 | 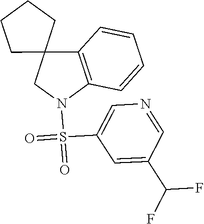 [SMILES: O=S(=O)(c1cncc(C(F)F)c1)N1CC2(CCCC2)c2ccccc21] | Yield: 94.4 mg, 54.9%; Appearance: Beige solid; ¹H NMR (600 MHz, DMSO-d₆) δ 9.14 (d, J = 2.2 Hz, 1H), 9.04 (q, J = 1.4 Hz, 1H), 8.38 (t, J = 1.6 Hz, 1H), 7.49 (d, J = 8.1 Hz, 1H), 7.22 (td, J = 7.8, 1.3 Hz, 1H), 7.20 - 7.09 (m, 2H), 7.04 (td, J = 7.5, 1.0 Hz, 1H), 3.79 (s, 2H), 1.69 (ddt, J = 9.3, 6.4, 3.7 Hz, 2H), 1.67 - 1.59 (m, 2H), 1.54 (tdd, J = 12.4, 5.5, 2.7 Hz, 2H), 1.51 - 1.41 (m, 2H); HPLC purity: 100%; LCMS Calculated for C₁₈H₁₈F₂N₂O₂S: 364.41; Observed: 365.2 [M + H]⁺. |
| I-21 | 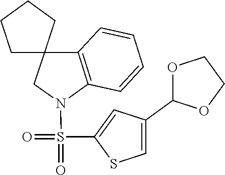 [SMILES: O=S(=O)(c1cc(C2OCCO2)cs1)N1CC2(CCCC2)c2ccccc21] | Yield: 60.8 mg, 40.8%; Appearance: Light brown solid; ¹H NMR (400 MHz, DMSO-d₆) δ 7.80 (d, J = 1.6 Hz, 1H), 7.56 (d, J = 1.5 Hz, 1H), 7.46 (d, J = 8.0 Hz, 1H), 7.25 - 7.15 (m, 1H), 7.10 (d, J = 7.5 Hz, 1H), 7.03 (t, J = 7.5 Hz, 1H), 5.75 (s, 1H), 4.14 - 3.83 (m, 4H), 3.66 (s, 2H), 2.01 - 1.44 (m, 8H); HPLC purity: 96.10%; LCMS Calculated for C₁₉H₂₁NO₄S₂: 391.50; Observed: 390.3 [M + H]⁺. |
| I-22 | 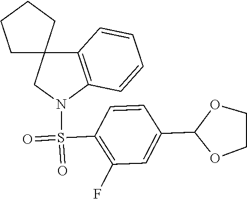 [SMILES: O=S(=O)(c1ccc(C2OCCO2)cc1F)N1CC2(CCCC2)c2ccccc21] | Yield: 49.0 mg, 31.8%; Appearance: Light brown solid; ¹H NMR (600 MHz, DMSO-d₆) δ 7.98 (ddd, J = 8.1, 6.9, 1.3 Hz, 1H), 7.49 - 7.37 (m, 2H), 7.26 (d, J = 8.1 Hz, 1H), 7.18 (dd, J = 7.5, 1.3 Hz, 1H), 7.13 (td, J = 7.8, 1.4 Hz, 1H), 7.00 (td, J = 7.5, 1.2 Hz, 1H), 5.78 (s, 1H), 4.06 - 3.96 (m, 2H), 3.96 - 3.89 (m, 2H), 3.81 (s, 2H), 1.72 (h, J = 4.7, 3.7 Hz, 2H), 1.64 (tq, J = 8.2, 5.1 Hz, 4H), 1.58 (dt, J = 8.1, 2.7 Hz, 2H); HPLC purity: 100%; LCMS Calculated for C₂₁H₂₂FNO₄S: 403.47; Observed: 404.2 [M + H]⁺. |
| I-23 | 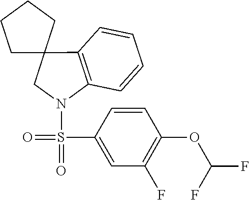 [SMILES: O=S(=O)(c1ccc(OC(F)F)c(F)c1)N1CC2(CCCC2)c2ccccc21] | Yield: 14.2 mg, 9.4%; Appearance: Beige oil; ¹H NMR (400 MHz, DMSO-d₆) δ 7.80 - 7.59 (m, 2H), 7.49 (d, J = 7.8 Hz, 2H), 7.26 - 7.13 (m, 2H), 7.09 (d, J = 7.5 Hz, 1H), 7.06 - 6.93 (m, 1H), 3.69 (s, 2H), 1.94 - 1.45 (m, 8H); HPLC purity: 98.15%; LCMS Calculated for C₁₉H₁₈F₃NO₃S: 397.41; Observed: 399.0 [M + 2H]⁺. |
| I-24 | 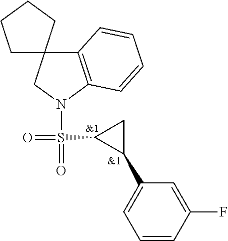 [SMILES: O=S(=O)([C@@H]1C[C@H]1c1cccc(F)c1)N1CC2(CCCC2)c2ccccc21] | Yield: 18.2 mg, 12.2%; Appearance: Beige solid; ¹H NMR (600 MHz, DMSO-d₆) δ 7.32 (d, J = 7.9 Hz, 1H), 7.25 - 7.19 (m, 2H), 7.17 (td, J = 7.8, 1.3 Hz, 1H), 7.01 (td, J = 7.5, 1.0 Hz, 1H), 6.96 (td, J = 8.6, 2.7 Hz, 1H), 6.88 (dt, J = 7.7, 1.2 Hz, 1H), 6.85 (dt, J = 10.4, 2.2 Hz, 1H), 3.84 - 3.75 (m, 2H), 3.18 (dt, J = 8.5, 5.1 Hz, 1H), 2.69 (ddd, J = 9.9, 6.5, 4.5 Hz, 1H), 1.75 - 1.69 (m, 5H), 1.65 (ddt, J = 17.7, 9.6, 5.8 Hz, 4H), 1.55 (dt, J = 8.4, 6.1 Hz, 1H); HPLC purity: 100%; LCMS Calculated for C₂₁H₂₂FNO₂S: 371.47; Observed: 372.2 [M + H]⁺. |
| I-25 | 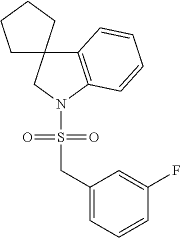 [SMILES: O=S(=O)(Cc1cccc(F)c1)N1CC2(CCCC2)c2ccccc21] | Yield: 13.4 mg, 9.0%; Appearance: Beige solid; ¹H NMR (400 MHz, DMSO-d₆) δ 7.37 (td, J = 7.9, 6.0 Hz, 1H), 7.24 (d, J = 7.5 Hz, 1H), 7.22 - 7.13 (m, 3H), 7.13 - 7.08 (m, 2H), 7.00 (ddd, J = 8.0, 5.2, 3.1 Hz, 1H), 4.66 (d, J = 1.8 Hz, 2H), 3.66 (d, J = 1.9 Hz, 2H), 1.94 - 1.52 (m, 8H); HPLC purity: 96.60%, LCMS Calculated for C₁₉H₂₀FNO₂S: 345.43; Observed: 344.2 [M − H]⁻. |
| I-26 | 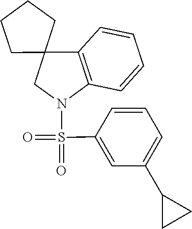 [SMILES: O=S(=O)(c1cccc(C2CC2)c1)N1CC2(CCCC2)c2ccccc21] | Yield: 99.5 mg, 63.3%; Appearance: Yellow solid; ¹H NMR (600 MHz, DMSO-d₆) δ 7.52 (dt, J = 7.7, 1.6 Hz, 1H), 7.46 (d, J = 8.1 Hz, 1H), 7.42 - 7.37 (m, 1H), 7.36 (dp, J = 4.4, 1.7 Hz, 2H), 7.20 (td, J = 7.8, 1.4 Hz, 1H), 7.13 (dd, J = 7.5, 1.4 Hz, 1H), 7.01 (td, J = 7.4, 1.1 Hz, 1H), 3.65 (d, J = 1.4 Hz, 2H), 1.96 (tt, J = 8.3, 5.0 Hz, 1H), 1.66 (dp, J = 9.3, 3.4, 2.9 Hz, 2H), 1.64 - 1.56 (m, 2H), 1.56 - 1.47 (m, 2H), 1.42 - 1.27 (m, 2H), 1.01 - 0.87 (m, 2H), 0.55 (qd, J = 5.3, 4.6, 1.4 Hz, 2H); HPLC purity: 99.00%; LCMS Calculated for C₂₁H₂₃NO₂S: 353.48; Observed: 354.2 [M + H]⁺. |
| I-27 | 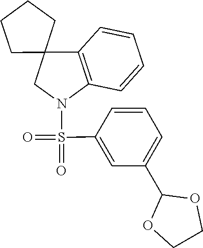 [SMILES: O=S(=O)(c1cccc(C2OCCO2)c1)N1CC2(CCCC2)c2ccccc21] | Yield: 95.1 mg, 60.8%; Appearance: Light brown oil; ¹H NMR (400 MHz, DMSO-d₆) δ 7.80 (s, 1H), 7.78 - 7.71 (m, 1H), 7.67 (d, J = 7.6 Hz, 1H), 7.53 (t, J = 7.5 Hz, 2H), 7.18 (t, J = 8.0 Hz, 1H), 7.05 (d, J = 7.9 Hz, 1H), 6.99 (t, J = 7.4 Hz, 1H), 5.78 (s, 1H), 3.97 (d, J = 1.4 Hz, 4H), 3.65 (s, 2H), 1.75 (d, J = 6.6 Hz, 2H), 1.72 - 1.66 (m, 2H), 1.66 - 1.55 (m, 2H), 1.47 (d, J = 12.2 Hz, 2H); HPLC purity: 100%; LCMS Calculated for C₂₁H₂₃NO₄S: 385.48; Observed: 386.2 [M + H]⁺. |
| I-28 | 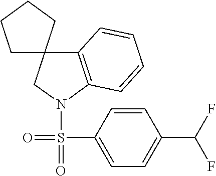 [SMILES: O=S(=O)(c1ccc(C(F)F)cc1)N1CC2(CCCC2)c2ccccc21] | Yield: 89.8 mg, 58.1%; Appearance: Beige solid; ¹H NMR (400 MHz, DMSO-d₆) δ 7.99 (d, J = 8.1 Hz, 2H), 7.78 (d, J = 8.0 Hz, 2H), 7.49 (d, J = 8.1 Hz, 1H), 7.31 - 7.20 (m, 1H), 7.17 (d, J = 7.5 Hz, 1H), 7.08 - 6.88 (m, 2H), 3.74 (s, 2H), 1.82 - 1.51 (m, 6H), 1.51 - 1.33 (m, 2H); HPLC purity: 100%; LCMS Calculated for C₁₉H₁₉F₂NO₂S: 363.42; Observed: 363.42 [M + H]⁺. |
| I-29 | 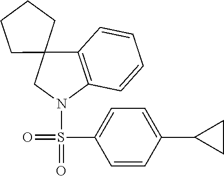 [SMILES: O=S(=O)(c1ccc(C2CC2)cc1)N1CC2(CCCC2)c2ccccc21] | Yield: 25.3 mg, 16.9%; Appearance: Beige solid; ¹H NMR (400 MHz, DMSO-d₆) δ 7.71 - 7.55 (m, 2H), 7.49 (d, J = 8.0 Hz, 1H), 7.28 - 7.09 (m, 3H), 7.05 (d, J = 7.5 Hz, 1H), 6.96 (t, J = 7.4 Hz, 1H), 3.60 (s, 2H), 1.94 (d, J = 8.2 Hz, 1H), 1.87 - 1.59 (m, 6H), 1.56 (d, J = 6.0 Hz, 2H), 1.04 (d, J = 7.9 Hz, 2H), 0.81 - 0.60 (m, 2H); HPLC purity: 100%; LCMS Calculated for C₂₁H₂₃NO₂S: 353.48; Observed: 354.1 [M + H]⁺. |
| I-30 | 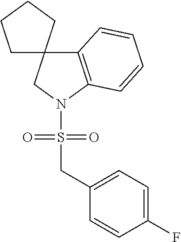 [SMILES: O=S(=O)(Cc1ccc(F)cc1)N1CC2(CCCC2)c2ccccc21] | Yield: 43.0 mg, 28.0%; Appearance: Pink solid; ¹H NMR (400 MHz, DMSO-d₆) δ 7.41 - 7.30 (m, 2H), 7.23 (d, J = 7.4 Hz, 1H), 7.16 (t, J = 8.9 Hz, 2H), 7.10 (dd, J = 7.7, 1.6 Hz, 2H), 7.03 - 6.94 (m, 1H), 4.62 (s, 2H), 3.66 (s, 2H), 1.92 - 1.54 (m, 8H); HPLC purity: 100%; LCMS Calculated for C₁₉H₂₀FNO₂S: 345.43; Observed: 345.43 [M + H]⁺. |
| I-31 | 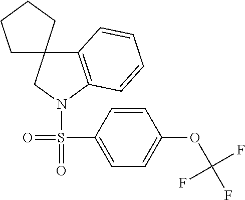 [SMILES: O=S(=O)(c1ccc(OC(F)(F)F)cc1)N1CC2(CCCC2)c2ccccc21] | Yield: 68.6 mg, 44.6%; Appearance: Beige solid; ¹H NMR (600 MHz, DMSO-d₆) δ 7.96 - 7.89 (m, 2H), 7.60 - 7.50 (m, 2H), 7.46 (d, J = 8.1 Hz, 1H), 7.20 (ddd, J = 8.3, 7.4, 1.2 Hz, 1H), 7.16 (dd, J = 7.5, 1.2 Hz, 1H), 7.02 (td, J = 7.5, 1.0 Hz, 1H), 3.70 (s, 2H), 1.67 (ddt, J = 12.3, 6.0, 3.5 Hz, 2H), 1.61 (qd, J = 8.3, 7.6, 4.3 Hz, 2H), 1.54 (dtd, J = 12.3, 7.7, 6.8, 2.9 Hz, 2H), 1.42 (ddd, J = 11.6, 6.0, 2.3 Hz, 2H); HPLC purity: 100%; LCMS Calculated for C₁₉H₁₈F₃NO₂S: 397.41; Observed: 397.41 [M − H]⁻. |
| I-32 | 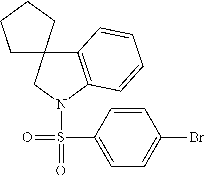 [SMILES: O=S(=O)(c1ccc(Br)cc1)N1CC2(CCCC2)c2ccccc21] | Yield: 24.3 mg, 16.4%; Appearance: Yellow solid; ¹H NMR (600 MHz, DMSO-d₆) δ 7.82 - 7.75 (m, 2H), 7.75 - 7.69 (m, 2H), 7.43 (d, J = 8.0 Hz, 1H), 7.19 (td, J = 7.8, 1.3 Hz, 1H), 7.15 (dd, J = 7.6, 1.3 Hz, 1H), 7.01 (td, J = 7.5, 1.0 Hz, 1H), 3.68 (s, 2H), 1.69 (dp, J = 9.9, 3.0 Hz, 2H), 1.67 - 1.60 (m, 2H), 1.56 (dddd, J = 14.2, 8.0, 5.8, 3.4 Hz, 2H), 1.52 - 1.42 (m, 2H); HPLC purity: 100%; LCMS Calculated for C₁₈H₁₈BrNO₂S: 392.31; Observed: 392.0 [M − H]⁻. |
| I-33 | 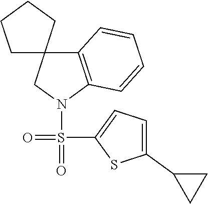 [SMILES: O=S(=O)(c1ccc(C2CC2)s1)N1CC2(CCCC2)c2ccccc21] | Yield: 47.1 mg, 30.3%; Appearance: Light brown solid; ¹H NMR (400 MHz, DMSO-d₆) δ 7.47 - 7.32 (m, 2H), 7.17 (td, J = 7.8, 1.6 Hz, 1H), 7.13 - 7.04 (m, 1H), 7.01 (t, J = 7.4 Hz, 1H), 6.78 (d, J = 3.7 Hz, 1H), 3.63 (d, J = 2.1 Hz, 2H), 2.10 (tt, J = 8.0, 4.9 Hz, 1H), 1.90 - 1.53 (m, 8H), 1.20 - 0.94 (m, 2H), 0.88 - 0.63 (m, 2H); HPLC purity: 100%; LCMS Calculated for C₁₉H₂₁NO₂S₂: 359.50; Observed: 360.0 [M + H]⁺. |
| I-34 | 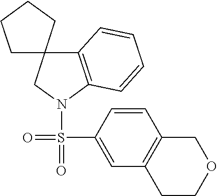 [SMILES: O=S(=O)(c1ccc2c(c1)CCOC2)N1CC2(CCCC2)c2ccccc21] | Yield: 9.8 mg, 6.4%; Appearance: Yellow solid; ¹H NMR (600 MHz, DMSO-d₆) δ 7.21 (d, J = 7.4 Hz, 1H), 7.12 - 7.03 (m, 4H), 7.00 - 6.93 (m, 1H), 6.83 (s, 1H), 4.50 (d, J = 4.1 Hz, 4H), 3.80 (t, J = 5.7 Hz, 2H), 3.59 (s, 2H), 2.71 (t, J = 5.7 Hz, 2H), 1.84 - 1.53 (m, 8H); HPLC purity: 100%; LCMS Calculated for C₂₂H₂₅NO₃S: 383.51; Observed: 383.51 [M + H]⁺. |
| I-35 | 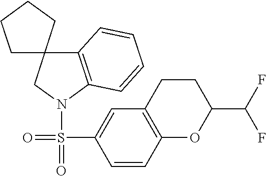 [SMILES: O=S(=O)(c1ccc2c(c1)CCC(C(F)F)O2)N1CC2(CCCC2)c2ccccc21] | Yield: 51.4 mg, 33.1%; Appearance: Yellow solid; ¹H NMR (600 MHz, DMSO-d₆) δ 7.62 (d, J = 2.1 Hz, 1H), 7.51 (dd, J = 8.7, 2.3 Hz, 1H), 7.41 (d, J = 8.1 Hz, 1H), 7.16 (tt, J = 7.9, 1.4 Hz, 1H), 7.13 (dd, J = 7.5, 1.3 Hz, 1H), 6.98 (td, J = 7.5, 1.1 Hz, 1H), 6.94 (dd, J = 8.8, 1.4 Hz, 1H), 6.20 (td, J = 54.5, 3.4 Hz, 1H), 4.44 (dt, J = 10.8, 3.1 Hz, 1H), 3.65 (d, J = 1.5 Hz, 2H), 2.87 - 2.75 (m, 2H), 2.10 - 2.01 (m, 1H), 1.77 - 1.68 (m, 3H), 1.63 (ddt, J = 12.3, 8.3, 4.5 Hz, 2H), 1.61 - 1.55 (m, 2H), 1.56 - 1.43 (m, 2H); HPLC purity: 98.77%; LCMS Calculated for |
| | | C₂₂H₂₃F₂NO₃S: 419.49; Observed: 420.0 [M + H]⁺. |
| I-36 | 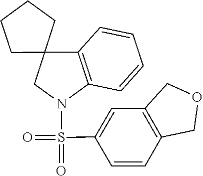 [SMILES: O=S(=O)(c1ccc2c(c1)COC2)N1CC2(CCCC2)c2ccccc21] | Yield: 17.2 mg, 11.6%; Appearance: White solid; ¹H NMR (600 MHz, DMSO-d₆) δ 7.80 (d, J = 1.6 Hz, 1H), 7.72 (dd, J = 7.9, 1.7 Hz, 1H), 7.47 (d, J = 8.0 Hz, 1H), 7.43 (d, J = 8.0 Hz, 1H), 7.17 (td, J = 7.8, 1.3 Hz, 1H), 7.13 (dd, J = 7.5, 1.3 Hz, 1H), 6.98 (td, J = 7.5, 1.0 Hz, 1H), 4.98 (s, 4H), 3.68 (s, 2H), 1.70 (qd, J = 8.5, 7.4, 3.5 Hz, 2H), 1.67 - 1.61 (m, 2H), 1.58 (ddd, J = 15.3, 8.5, 4.6 Hz, 2H), 1.51 (ddd, J = 11.7, 6.1, 2.7 Hz, 2H); HPLC purity: 100%; LCMS Calculated for C₂₀H₂₁NO₃S: 355.45; Observed: 356.1 [M + H]⁺. |
| I-37 | 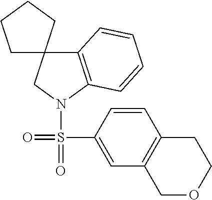 [SMILES: O=S(=O)(c1ccc2c(c1)COCC2)N1CC2(CCCC2)c2ccccc21] | Yield: 17.3 mg, 11.4%; Appearance: Brown solid; ¹H NMR (600 MHz, DMSO-d₆) δ 7.56 (d, J = 6.2 Hz, 2H), 7.41 (d, J = 8.0 Hz, 1H), 7.29 (d, J = 8.5 Hz, 1H), 7.16 (td, J = 7.8, 1.4 Hz, 1H), 7.13 (dd, J = 7.4, 1.3 Hz, 1H), 6.98 (t, J = 7.4 Hz, 1H), 4.67 (s, 2H), 3.81 (t, J = 5.7 Hz, 2H), 3.67 (s, 2H), 2.78 (t, J = 5.7 Hz, 2H), 1.76 - 1.67 (m, 2H), 1.67 - 1.61 (m, 2H), 1.58 (ddd, J = 14.4, 8.5, 4.3 Hz, 2H), 1.55 - 1.46 (m, 2H); HPLC purity: 100%; LCMS Calculated for C₂₁H₂₃NO₃S: 369.48; Observed: 369.4 [M − H]⁻. |
| I-38 | 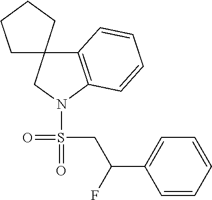 [SMILES: O=S(=O)(CC(F)c1ccccc1)N1CC2(CCCC2)c2ccccc21] | Yield: 17.0 mg, 10.9%; Appearance: Pink solid; ¹H NMR (400 MHz, DMSO-d₆) δ 7.60 (dd, J = 6.4, 3.0 Hz, 2H), 7.52 (d, J = 15.5 Hz, 1H), 7.43 - 7.24 (m, 4H), 7.22 - 7.06 (m, 3H), 6.97 (t, J = 7.2 Hz, 1H), 3.74 (s, 2H), 1.83 (d, J = 16.5 Hz, 8H); HPLC purity: 100%; LCMS Calculated for C₂₀H₂₂FNO₂S: 359.46; Observed: 359.46 [M − H]⁻. |
| I-39 | 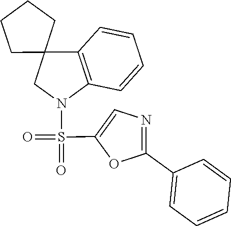 [SMILES: O=S(=O)(c1cnc(-c2ccccc2)o1)N1CC2(CCCC2)c2ccccc21] | Yield: 26.3 mg, 17.6%; Appearance: Light brown solid; ¹H NMR (600 MHz, DMSO-d₆) δ 8.17 (s, 1H), 7.86 (d, J = 7.6 Hz, 2H), 7.58 (t, J = 7.4 Hz, 1H), 7.52 (t, J = 7.5 Hz, 2H), 7.44 (d, J = 8.0 Hz, 1H), 7.25 (t, J = 7.8 Hz, 1H), 7.21 (d, J = 7.5 Hz, 1H), 7.08 (t, J = 7.5 Hz, 1H), 3.86 (s, 2H), 1.84 - 1.51 (m, 8H); HPLC purity: 100%; LCMS Calculated for C₂₁H₂₀N₂O₃S: 380.46; Observed: 381.2 [M + H]⁺. |
| I-40 | 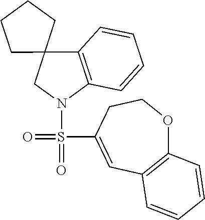 [SMILES: O=S(=O)(C1=Cc2ccccc2OCC1)N1CC2(CCCC2)c2ccccc21] | Yield: 18.7 mg, 15.2%; Appearance: Brown solid; ¹H NMR (400 MHz, DMSO-d₆) δ 7.47 (d, J = 7.7 Hz, 1H), 7.43 (s, 1H), 7.33 (d, J = 8.1 Hz, 1H), 7.27 (d, J = 7.9 Hz, 1H), 7.19 - 7.09 (m, 2H), 7.06 (t, J = 7.6 Hz, 1H), 6.98 (t, J = 7.6 Hz, 1H), 6.92 (d, J = 8.3 Hz, 1H), 4.18 (d, J = 4.7 Hz, 2H), 3.69 (d, J = 2.6 Hz, 2H), 2.78 (d, J = 5.1 Hz, 2H), 1.77 (d, J = 32.2 Hz, 8H); HPLC purity: 100%; LCMS Calculated for C₂₂H₂₃NO₃S: 381.49; Observed: 382.2 [M + H]⁺. |
| I-41 | 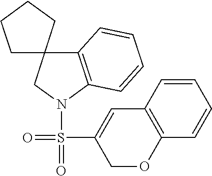 [SMILES: O=S(=O)(C1=Cc2ccccc2OC1)N1CC2(CCCC2)c2ccccc21] | Yield: 14.0 mg, 9.4%; Appearance: Beige solid; ¹H NMR (600 MHz, DMSO-d₆) δ 7.56 (s, 1H), 7.41 (dd, J = 7.6, 1.5 Hz, 1H), 7.33 - 7.25 (m, 2H), 7.24 (d, J = 7.5 Hz, 1H), 7.21 - 7.13 (m, 1H), 7.04 (ddd, J = 8.4, 7.0, 1.1 Hz, 1H), 6.98 (tt, J = 7.5, 1.0 Hz, 1H), 6.82 (d, J = 8.2 Hz, 1H), 4.73 (d, J = 1.1 Hz, 2H), 3.76 (d, J = 1.0 Hz, 2H), 1.91 - 1.48 (m, 8H), HPLC purity: 100%; LCMS Calculated for C₂₁H₂₁NO₃S: 367.46; Observed: 368.2 [M + H]⁺. |
| I-42 | 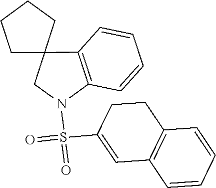 [SMILES: O=S(=O)(C1=Cc2ccccc2CC1)N1CC2(CCCC2)c2ccccc21] | Yield: 29.0 mg, 19.4%; Appearance: Beige solid; ¹H NMR (600 MHz, DMSO-d₆) δ 7.48 (s, 1H), 7.41 (dd, J = 7.4, 1.4 Hz, 1H), 7.31 (d, J = 8.1 Hz, 1H), 7.27 (td, J = 7.4, 1.5 Hz, 1H), 7.25 - 7.18 (m, 2H), 7.15 (td, J = 7.8, 1.4 Hz, 2H), 6.99 (t, J = 7.4 Hz, 1H), 3.71 (s, 2H), 2.74 (t, J = 8.3 Hz, 2H), 2.40 - 2.30 (m, 2H), 1.81 - 1.58 (m, 8H); HPLC purity: 100%; LCMS Calculated for C₂₂H₂₃NO₂S: 365.49; Observed: 366.1 [M + H]⁺. |
| I-43 | 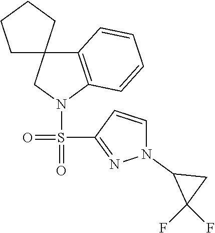 [SMILES: O=S(=O)(c1ccn(C2CC2(F)F)n1)N1CC2(CCCC2)c2ccccc21] | Yield: 89.9 mg, 59.7%; Appearance: Yellow oil; ¹H NMR (400 MHz, DMSO-d₆) δ 7.99 (d, J = 2.3 Hz, 1H), 7.40 (d, J = 8.1 Hz, 1H), 7.20 - 7.09 (m, 1H), 7.07 (d, J = 7.4 Hz, 1H), 6.96 (t, J = 7.4 Hz, 1H), 6.63 (d, J = 2.3 Hz, 1H), 4.56 (q, J = 8.5 Hz, 1H), 3.75 (s, 2H), 2.45 - 2.30 (m, 1H), 2.22 (dt, J = 17.5, 10.3 Hz, 1H), 1.95 - 1.45 (m, 8H); HPLC purity: 100%; LCMS Calculated for C₁₈H₁₉F₂N₃O₂S: 379.43; Observed: 380.2 [M + H]⁺. |
| I-44 | 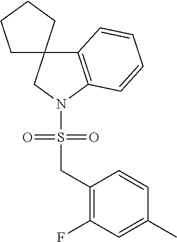 [SMILES: Cc1ccc(CS(=O)(=O)N2CC3(CCCC3)c3ccccc32)c(F)c1] | Yield: 31.9 mg, 20.9%; Appearance: Pink solid; ¹H NMR (600 MHz, DMSO-d₆) δ 7.25 (t, J = 7.9 Hz, 1H), 7.21 (dd, J = 7.4, 1.2 Hz, 1H), 7.07 (ddd, J = 8.4, 7.2, 1.3 Hz, 1H), 7.03 (dd, J = 8.1, 1.2 Hz, 1H), 7.00 - 6.93 (m, 3H), 4.53 (s, 2H), 3.65 (s, 2H), 2.26 (s, 3H), 1.75 (qd, J = 7.1, 4.3, 3.2 Hz, 6H), 1.70 - 1.57 (m, 2H); HPLC purity: 100%; LCMS Calculated for C₂₀H₂₂FNO₂S: 359.46; Observed: 359.46 [M − H]⁻. |
| I-45 | 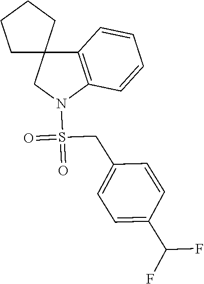 [SMILES: O=S(=O)(Cc1ccc(C(F)F)cc1)N1CC2(CCCC2)c2ccccc21] | Yield: 21.5 mg, 14.1%; Appearance: Beige solid; ¹H NMR (600 MHz, DMSO-d₆) δ 7.50 (d, J = 7.9 Hz, 2H), 7.44 (d, J = 7.9 Hz, 2H), 7.21 (d, J = 7.5 Hz, 1H), 7.13 - 7.03 (m, 2H), 7.01 - 6.87 (m, 2H), 4.65 (s, 2H), 3.63 (s, 2H), 1.74 (ddd, J = 17.8, 7.4, 4.0 Hz, 6H), 1.69 - 1.54 (m, 2H); HPLC purity: 100%; LCMS Calculated for C₂₀H₂₁F₂NO₂S: 377.45; Observed: 376.2 [M − H]⁻. |
| I-46 | 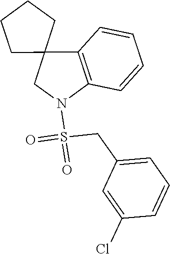 [SMILES: O=S(=O)(Cc1cccc(Cl)c1)N1CC2(CCCC2)c2ccccc21] | Yield: 15.5 mg, 10.4%; Appearance: Beige solid; ¹H NMR (600 MHz, DMSO-d₆) δ 7.37 (dt, J = 8.2, 1.6 Hz, 1H), 7.35 - 7.30 (m, 2H), 7.28 (dt, J = 7.5, 1.4 Hz, 1H), 7.21 (d, J = 7.5 Hz, 1H), 7.09 (d, J = 4.1 Hz, 2H), 7.01 - 6.94 (m, 1H), 4.63 (s, 2H), 3.64 (s, 2H), 1.88 - 1.70 (m, 6H), 1.65 (dd, J = 10.1, 6.5 Hz, 2H); HPLC purity: 100%; LCMS Calculated for C₁₉H₂₀ClNO₂S: 361.88; Observed: 361.88 [M − H]⁻. |
| I-47 | 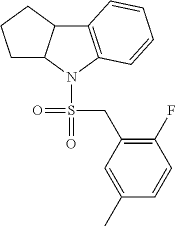 | Yield: 25.5 mg, 16.4%; Appearance: Yellow oil; ¹H NMR (400 MHz, DMSO-d₆) δ 7.12 (s, 3H), 7.06 (d, J = 6.1 Hz, 2H), 6.93 (dt, J = 16.9, 7.8 Hz, 2H), 4.44 (s, 2H), 3.65 (s, 2H), 2.27 (s, 3H), 1.78 (d, J = 35.8 Hz, 8H); HPLC purity: 100%; LCMS Calculated for C₂₀H₂₂FNO₂S: 359.46; Observed: 359.46 [M − H]⁻. |
| I-48 | 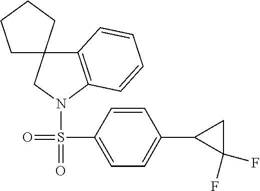 [SMILES: O=S(=O)(c1ccc(C2CC2(F)F)cc1)N1CC2(CCCC2)c2ccccc21] | Yield: 36.3 mg, 24.3%; Appearance: Beige solid; ¹H NMR (400 MHz, DMSO-d₆) δ 7.82 - 7.62 (m, 2H), 7.51 (d, J = 8.1 Hz, 1H), 7.48 - 7.35 (m, 2H), 7.17 (t, J = 7.7 Hz, 1H), 7.06 (d, J = 7.5 Hz, 1H), 6.98 (t, J = 7.4 Hz, 1H), 3.59 (s, 2H), 2.94 (dd, J = 12.3, 8.5 Hz, 1H), 2.07 - 1.82 (m, 2H), 1.82 - 1.57 (m, 6H), 1.58 - 1.44 (m, 2H); HPLC purity: 100%; LCMS Calculated for C₂₁H₂₁F₂NO₂S: 389.46; Observed: 390.2 [M + H]⁺. |
| I-49 | 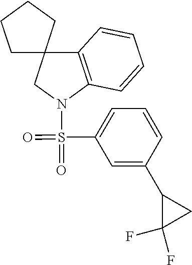 [SMILES: O=S(=O)(c1cccc(C2CC2(F)F)c1)N1CC2(CCCC2)c2ccccc21] | Yield: 63.6 mg, 42.4%; Appearance: Grey solid; ¹H NMR (500 MHz, DMSO-d₆) δ 7.69 - 7.58 (m, 2H), 7.50 (dd, J = 8.1, 4.7 Hz, 2H), 7.45 (td, J = 7.7, 1.8 Hz, 1H), 7.16 (t, J = 7.8 Hz, 1H), 7.03 (d, J = 7.6 Hz, 1H), 6.97 (t, J = 7.4 Hz, 1H), 3.63 (s, 2H), 2.95 (dd, J = 12.7, 8.4 Hz, 1H), 1.98 - 1.85 (m, 1H), 1.74 (tdd, J = 13.5, 7.2, 3.2 Hz, 3H), 1.69 - 1.63 (m, 2H), 1.58 (dq, J = 15.3, 7.7 Hz, 2H), 1.46 (td, J = 12.5, 11.4, 6.0 Hz, 2H); HPLC purity: 95.83%; LCMS Calculated for C₂₁H₂₁F₂NO₂S: 389.46; Observed: 390.2 [M + H]⁺. |

### Example 2—Compounds Prepared According to Method 2

The following compounds were prepared according to Method 2. A person of skill in the art would understand what sulfonyl chloride compounds having certain values for variable R would be used in the generic scheme to prepare the compounds provided below, using standard chemical manipulations and procedures similar to those used for the preparation of Method 2.

| Compound | | |
|---|---|---|
| No. | Structure | Analytical data |
| I-50 | 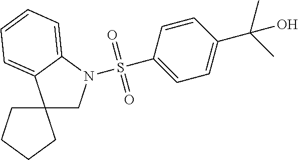 [SMILES: CC(C)(O)c1ccc(S(=O)(=O)N2CC3(CCCC3)c3ccccc32)cc1] | ¹H NMR (400 MHz, DMSO-d₆) δ 1.36 (s, 6H) 1.42-1.75 (m, 8H) 3.67 (s, 2 H) 5.19 (br s, 1 H) 6.98-7.02 (m, 1 H) 7.12-7.20 (m, 2 H) 7.45 (d, J = 7.34 Hz, 1 H) 7.62 (d, J = 6.85 Hz, 2 H) 7.73 (d, J = 6.36 Hz, 2 H); HPLC purity: 99.81%; LCMS Calculated for C₂₁H₂₅NO₃S: 371.16; Observed: 372.0 [M + H]⁺. |
| I-51 | 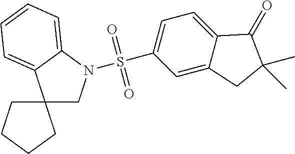 [SMILES: CC1(C)Cc2cc(S(=O)(=O)N3CC4(CCCC4)c4ccccc43)ccc2C1=O] | ¹H NMR (400 MHz, DMSO-d₆) δ 1.11 (s, 6 H) 1.43-1.76 (m, 8H) 3.06 (s, 2 H) 3.77 (s, 2 H) 7.01-7.07 (m, 1 H) 7.18 (d, J = 7.34 Hz, 1 H) 7.23 (t, J = 7.58 Hz, 1 H) 7.49 (d, J = 7.83 Hz, 1 H) 7.77-7.84 (m, 2 H) 8.08 (s, 1 H); HPLC purity: 99.63%; LCMS Calculated for C₂₃H₂₅NO₃S: 395.16; Observed: 396.0 [M + H]⁺. |
| I-52 | 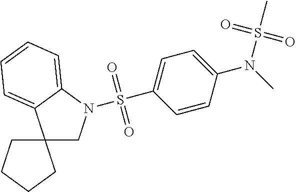 [SMILES: CN(c1ccc(S(=O)(=O)N2CC3(CCCC3)c3ccccc32)cc1)S(C)(=O)=O] | ¹H NMR (400 MHz, DMSO-d₆) δ 1.42-1.51 (m, 2 H) 1.53-1.76 (m, 6 H) 2.99 (s, 3 H) 3.25 (s, 3 H) 3.71 (s, 2 H) 7.01-7.06 (m, 1 H) 7.14- 7.25 (m, 2 H) 7.48 (d, J = 8.31 Hz, 1 H) 7.57 (d, J = 8.80 Hz, 2 H) 7.83 (d, J = 8.80 Hz, 2 H); HPLC purity: 99.85%; LCMS Calculated for C₂₀H₂₄N₂O₄S₂: 420.12; Observed: 421.0 [M + H]⁺. |
| I-53 | 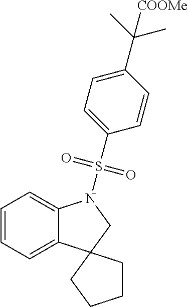 [SMILES: COC(=O)C(C)(C)c1ccc(S(=O)(=O)N2CC3(CCCC3)c3ccccc32)cc1] | ¹H NMR (400 MHz, DMSO-d₆) δ 1.48 (s, 6 H) 1.51-1.79 (m, 8H) 3.56 (s, 3 H) 3.69 (s, 2 H) 7.00-7.07 (m, 1 H) 7.16 (d, J = 7.34 Hz, 1 H) 7.21 (t, J = 7.83 Hz, 1 H) 7.45-7.53 (m, 3 H) 7.78 (d, J = 8.31 Hz, 2 H); HPLC purity: 99.67%; LCMS Calculated for C₂₃H₂₇NO₄S: 413.17; Observed: 414.01 [M + H]⁺. |
| I-54 | 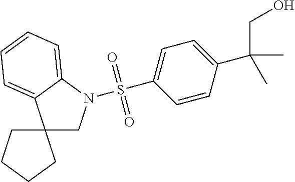 [SMILES: CC(C)(CO)c1ccc(S(=O)(=O)N2CC3(CCCC3)c3ccccc32)cc1] | ¹H NMR (400 MHz, DMSO-d₆) δ 1.18 (s, 6 H) 1.45-1.76 (m, 8H) 3.40 (d, J = 4.89 Hz, 2 H) 3.68 (s, 2 H) 4.73 (t, J = 5.38 Hz, 1 H) 7.02 (d, J = 7.34 Hz, 1 H) 7.15-7.23 (m, 2 H) 7.47 (d, J = 8.31 Hz, 1 H) 7.56 (d, J = 8.31 Hz, 2 H) 7.74 (d, J = 8.80 Hz, 2 H); HPLC purity: 99.81%; LCMS Calculated for C₂₂H₂₇NO₃S: 385.17; Observed: 385.9 [M]⁺. |
| I-55 | 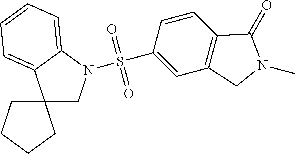 [SMILES: CN1Cc2cc(S(=O)(=O)N3CC4(CCCC4)c4ccccc43)ccc2C1=O] | ¹H NMR (400 MHz, DMSO-d₆) δ 1.41-1.74 (m, 8 H) 3.05 (s, 3 H) 3.74 (s, 2 H) 4.50 (s, 2 H) 6.99-7.05 (m, 1 H) 7.16 (d, J = 7.34 Hz, 1 H) 7.21 (t, J = 7.83 Hz, 1 H) 7.48 (d, J = 7.82 Hz, 1 H) 7.77-7.83 (m, 1 H) 7.90 (d, J = 7.83 Hz, 1 H) 8.13 (s, 1 H); HPLC purity: 99.33%; LCMS Calculated for C₂₁H₂₂N₂O₃S: 382.14; Observed: 383.30 [M + H]⁺. |
| I-56 | 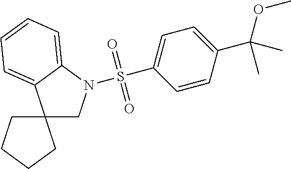 [SMILES: COC(C)(C)c1ccc(S(=O)(=O)N2CC3(CCCC3)c3ccccc32)cc1] | ¹H NMR (400 MHz, DMSO-d₆) δ 1.40 (s, 6 H) 1.48-1.74 (m, 8H) 2.94 (s, 3 H) 3.69 (s, 2 H) 7.01-7.07 (m, 1 H) 7.16 (d, J = 6.85 Hz, 1 H) 7.22 (t, J = 7.83 Hz, 1 H) 7.49 (d, J = 7.83 Hz, 1 H) 7.57 (d, J = 8.31 Hz, 2 H) 7.78 (d, J = 8.31 Hz, 2 H); HPLC purity: 96.89%; LCMS Calculated for C₂₂H₂₇NO₃S: 385.17; Observed: 385.9 [M]⁺. |
| I-57 | 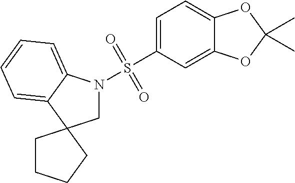 [SMILES: CC1(C)Oc2ccc(S(=O)(=O)N3CC4(CCCC4)c4ccccc43)cc2O1] | ¹H NMR (400 MHz, DMSO-d₆) δ 1.50-1.54 (m, 2 H) 1.57-1.62 (m, 2 H) 1.64 (s, 6H) 1.66-1.76 (m, 4H) 3.68 (s, 2 H) 6.98-7.05 (m, 2 H) 7.16- 7.24 (m, 3 H) 7.36 (d, J = 8.31 Hz, 1 H) 7.45 (d, J = 8.31 Hz, 1 H); HPLC purity: 99.81%; LCMS Calculated for C₂₁H₂₃NO₄S: 385.13; Observed: 386.04 [M + H]⁺. |
| I-58 | 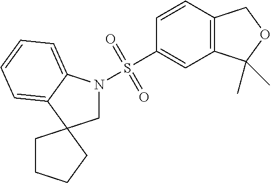 [SMILES: CC1(C)OCc2ccc(S(=O)(=O)N3CC4(CCCC4)c4ccccc43)cc21] | ¹H NMR (400 MHz, DMSO-d₆) δ 1.34-1.36 (m, 1 H) 1.38 (s, 6 H) 1.49-1.55 (m, 3H) 1.60-1.74 (m, 4 H) 3.74 (s, 2 H) 4.95 (s, 2 H) 6.98-7.06 (m, 1 H) 7.14 (d, J = 7.34 Hz, 1 H) 7.21 (t, J = 7.58 Hz, 1 H) 7.41 (d, J = 7.83 Hz, 1 H) 7.51 (d, J = 8.31 Hz, 1 H) 7.64 (d, J = 7.83 Hz, 1 H) 7.77 (s, 1 H); HPLC purity: 97.01%; LCMS Calculated for C₂₂H₂₅NO₃S: 383.16; Observed: 383.95 [M]⁺. |
| I-59 | 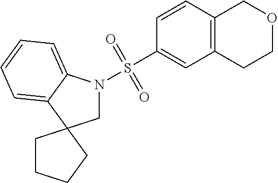 [SMILES: O=S(=O)(c1ccc2c(c1)CCOC2)N1CC2(CCCC2)c2ccccc21] | ¹H NMR (400 MHz, DMSO-d₆) δ 1.47-1.80 (m, 8H) 2.78-2.88 (m, 2 H) 3.70 (s, 2 H) 3.84 (t, J = 5.62 Hz, 2 H) 4.69 (s, 2 H) 6.97-7.05 (m, 1 H) 7.13-7.24 (m, 3 H) 7.45 (d, J = 7.82 Hz, 1 H) 7.58 (d, J = 7.82 Hz, 1 H) 7.65 (s, 1 H); HPLC purity: 99.33%; LCMS Calculated for C₂₁H₂₃NO₃S: 369.14; Observed: 369.95 [M + H]⁺. |
| I-60 | 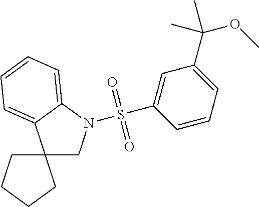 [SMILES: COC(C)(C)c1cccc(S(=O)(=O)N2CC3(CCCC3)c3ccccc32)c1] | ¹H NMR (400 MHz, DMSO-d₆) δ 1.25-1.31 (m, 2 H) 1.34 (s, 6H) 1.44-1.51 (m, 2 H) 1.56- 1.72 (m, 4 H) 2.81 (s, 3 H) 3.69 (s, 2 H) 7.03- 7.07 (m, 1 H) 7.14 (d, J = 7.83 Hz, 1 H) 7.25 (t, J = 7.58 Hz, 1 H) 7.51-7.59 (m, 2 H) 7.64-7.72 (m, 3 H); HPLC purity: 98.50%; LCMS Calculated for C₂₂H₂₃NO₃S: 385.17; Observed: 386.05 [M + H]⁺. |
| I-61 | 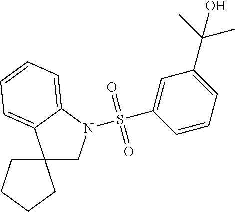 [SMILES: CC(C)(O)c1cccc(S(=O)(=O)N2CC3(CCCC3)c3ccccc32)c1] | ¹H NMR (400 MHz, DMSO-d₆) δ 1.33 (s, 6 H) 1.34-1.37 (m, 1 H) 1.45-1.56 (m, 3H) 1.57- 1.73 (m, 4 H) 3.69 (s, 2 H) 5.27 (s, 1 H) 7.01- 7.06 (m, 1 H) 7.14 (d, J = 7.34 Hz, 1 H) 7.22 (t, J = 7.58 Hz, 1 H) 7.46-7.53 (m, 2 H) 7.59 (d, J = 7.83 Hz, 1 H) 7.73 (d, J = 7.83 Hz, 1 H) 7.84 (s, 1 H); HPLC purity: 99.85%; LCMS Calculated for C₂₁H₂₅NO₃S: 371.16; Observed: 371.87 [M]⁺. |
| I-62 | 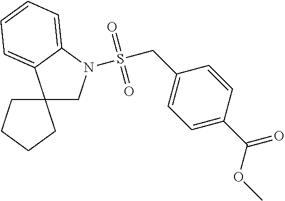 [SMILES: COC(=O)c1ccc(CS(=O)(=O)N2CC3(CCCC3)c3ccccc32)cc1] | ¹H NMR (400 MHz, CDCl₃) δ 1.65-1.74 (m, 4 H) 1.76-1.87 (m, 4 H) 3.53 (s, 2 H) 3.93 (s, 3 H) 4.43 (s, 2 H) 7.03-7.08 (m, 1 H) 7.14-7.22 (m, 2 H) 7.31 (d, J = 7.83 Hz, 1 H) 7.36 (d, J = 7.83 Hz, 2 H) 7.99 (d, J = 8.31 Hz, 2 H); HPLC purity: 99.75%; LCMS Calculated for C₂₁H₂₃NO₄S: 385.13; Observed: 403.31 [M + 18]⁺. |
| I-63 | 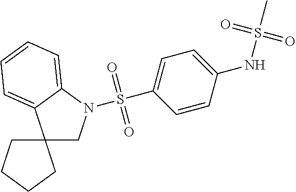 [SMILES: CS(=O)(=O)Nc1ccc(S(=O)(=O)N2CC3(CCCC3)c3ccccc32)cc1] | ¹H NMR (400 MHz, DMSO-d₆) δ 1.43-1.82 (m, 8 H) 3.09 (s, 3 H) 3.68 (s, 2 H) 6.97-7.08 (m, 1 H) 7.15-7.24 (m, 2 H) 7.29 (d, J = 8.31 Hz, 2 H) 7.45 (d, J = 7.83 Hz, 1 H) 7.78 (d, J = 8.31 Hz, 2 H) 10.44 (br s, 1 H); HPLC purity: 99.80%; LCMS Calculated for C₁₉H₂₂N₂O₄S₂: 406.10; Observed: 407.0 [M + H]⁺. |
| I-64 | 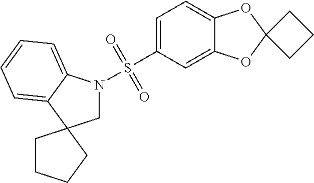 [SMILES: O=S(=O)(c1ccc2c(c1)OC1(CCC1)O2)N1CC2(CCCC2)c2ccccc21] | ¹H NMR (400 MHz, DMSO-d₆) δ 1.48-1.85 (m, 10H) 2.56-2.60 (m, 4 H) 3.67 (s, 2 H) 6.98- 7.08 (m, 2 H) 7.14-7.23 (m, 2 H) 7.28 (s, 1 H) 7.40 (d, J = 8.31 Hz, 1 H) 7.44 (d, J = 8.31 Hz, 1 H); HPLC purity: 98.95%; LCMS Calculated for C₂₂H₂₃NO₄S: 397.13; Observed: 398.25 [M + H]⁺. |

### Example 3—Synthesis of 1′-(4-chloro-2-fluorophenyl)-1-((4-(methylsulfonyl)phenyl)sulfonyl)spiro[indoline-3,4′-piperidine](I-65)

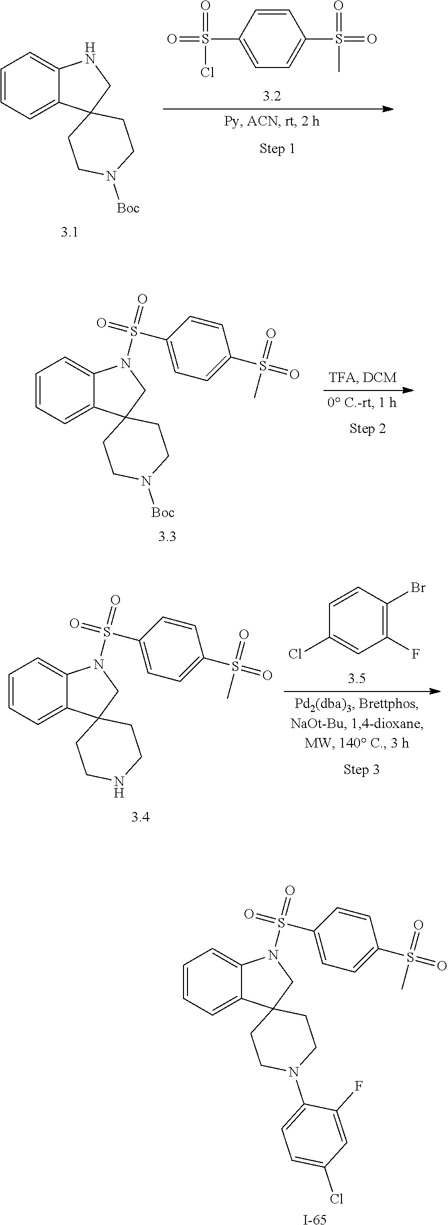

### Step-1: Procedure for Synthesis of tert-butyl(methylsulfonyl)phenyl)sulfonyl)spiro[indoline-3,4′-piperidine]-1′-carboxylate 3.3

To a stirred solution of tert-butyl spiro[indoline-3,4′-piperidine]-1′-carboxylate 3.1 (1 g, 3.46 mmol, 1 eq.) and 4-(methylsulfonyl)benzenesulfonyl chloride 3.2 (971 mg, 3.81 mmol, 1.1 eq.) in acetonitrile (10 mL), pyridine (0.83 mL, 10.4 mmol, 3 eq.) was added at room temperature and the reaction mixture was stirred for 2 h. The progress of the reaction was monitored by TLC. After completion of reaction, the reaction mixture was concentrated under reduced pressure. The residue was dissolved in ethyl acetate and washed with water. The organic layer was dried over anhydrous Na₂SO₄, filtered and concentrated under reduced pressure. The crude product was purified by column chromatography on silica gel to afford tert-butyl 1-((4-(methylsulfonyl)phenyl)sulfonyl)spiro[indoline-3,4′-piperidine]-1′-carboxylate 3.3 (850 mg, 50%). LCMS: 507.15 [M+H]⁺.

### Step-2: Procedure for Synthesis of 1-((4-(methylsulfonyl)phenyl)sulfonyl)spiro[indoline-3,4′-piperidine] 3.4

To a stirred solution of tert-butyl 1-((4-(methylsulfonyl)phenyl)sulfonyl)spiro[indoline-3,4′-piperidine]-1′-carboxylate 3.3 (850 mg, 1.67 mmol, 1 eq.) in DCM (10 mL), trifluoroacetic acid (5 mL) was added at 0° C. The reaction mixture was warmed to room temperature and stirred for 1 h. The progress of the reaction was monitored by TLC. After completion of the reaction, the reaction mixture was concentrated under reduced pressure. The residue was dissolved in saturated aqueous NaHCO₃ solution and extracted with ethyl acetate. The combined organic layers were dried over anhydrous Na₂SO₄, filtered and concentrated under reduced pressure to dryness to afford 1-((4-(methylsulfonyl)phenyl)sulfonyl)spiro[indoline-3,4′-piperidine] 3.4 (600 mg, crude). This compound was used in the next step without further purification. LCMS: 407.10 [M+H]⁺.

### Step-3: Procedure for Synthesis of 1′-(4-chloro-2-fluorophenyl)-1-((4-(methylsulfonyl)phenyl)sulfonyl)spiro[indoline-3,4′-piperidine]

A microwave tube was charged with a solution of 1-((4-(methylsulfonyl)phenyl)sulfonyl)spiro[indoline-3,4′-piperidine] 3.4 (300 mg, 0.74 mmol, 1 eq), 1-bromo-4-chloro-2-fluorobenzene 3.5 (230 mg, 1.1 mmol, 1.5 eq) and sodium tert-butoxide (141 mg, 1.47 mmol, 2 eq) in 1,4-dioxane (10 mL). The tube was sealed with a septum and the reaction mixture was purged with argon for 10 min. Tris(dibenzylideneacetone)dipalladium(0) (47 mg, 0.052 mmol, 0.07 eq) and Brettphos (59 mg, 0.11 mmol, 0.15 eq) were added to the reaction mixture under an argon atmosphere. The tube was then sealed with an aluminium cap and the reaction mixture was irradiated in a microwave reactor at 140° C. for 3 h. The progress of the reaction was monitored by TLC. After completion of the reaction, the reaction mixture was cooled to room temperature, filtered through a pad of Celite and the Celite pad was washed with ethyl acetate. The filtrate was concentrated under reduced pressure and the crude product was purified by column chromatography on silica gel followed by reverse phase preparative HPLC to afford 1′-(4-chloro-2-fluorophenyl)-1-((4-(methylsulfonyl)phenyl)sulfonyl)spiro[indoline-3,4′-piperidine]1-65.

Yield: 43 mg, 10.9%; Appearance: White solid; ¹H NMR (400 MHz, DMSO-d₆) δ 8.13 (s, 4H), 7.53 (d, J=8.0 Hz, 1H), 7.36-7.24 (m, 3H), 7.21 (d, J=8.4 Hz, 1H), 7.14-7.05 (m, 2H), 3.97 (s, 2H), 3.28 (s, 3H), 3.20 (d, J=12.0 Hz, 2H), 2.80 (t, J=11.6 Hz, 2H), 1.86 (t, J=11.2 Hz, 2H), 1.25 (d, J=12.8 Hz, 2H): HPLC purity: 96.55%; LCMS calculated for C₂₅H₂₄ClFN₂O₄S₂: 534.09; Observed: 535.20 [M+H]⁺.

### Example 4(1)—Synthesis of 1′-((1,1-dimethyl-1,3-dihydroisobenzofuran-5-yl)sulfonyl)spiro[cyclopentane-1,3′-indoline] (I-66)

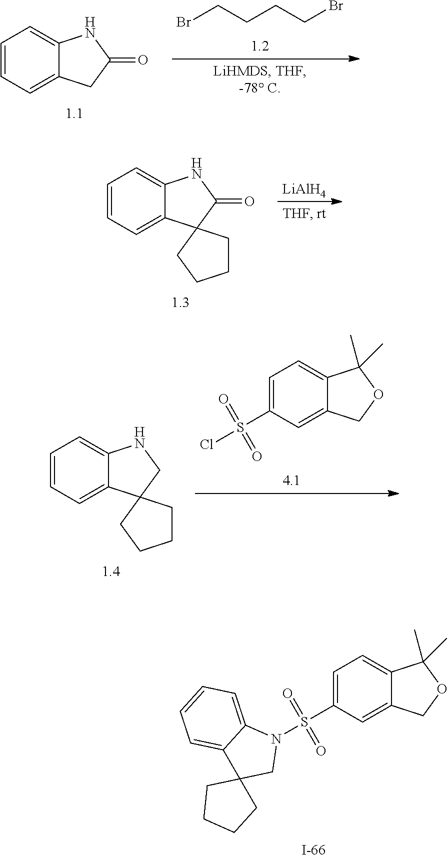

### Step-1: Procedure for Synthesis of spiro[cyclopentane-1,3′-indolin]-2′-one 1.3

To a solution of indolin-2-one 1.1 (20 g, 150 mmol) in THF (200 mL) cooled to −78° C., was added LiHMDS (1.0 M in THF, 300 mL, 300 mmol) dropwise. It was slowly warmed to −50° C. and stirred for 30 min, followed by cooling to −78° C. and 1,5-dibromobutane 1.2 (35.7 g, 165 mmol) was added. The reaction mixture was stirred at room temperature for 3 h. The progress of the reaction was monitored by TLC. After completion of reaction as monitored by TLC, the reaction mixture was quenched with saturated solution of ammonium chloride and extracted with ethyl acetate. The combined organic layers were washed with water, brine, dried over anhydrous sodium sulfate and concentrated under reduced pressure to dryness to afford spiro[cyclopentane-1,3′-indolin]-2′-one 1.3 (16 g, crude). This compound was used in the next step without further purification. LCMS: 188.0 [M+H].

### Step-2: Procedure for Synthesis of spiro[cyclopentane-1,3′-indoline 1.4

To a solution of spiro[cyclopentane-1,3′-indolin]-2′-one 1.3 (16 g, 85.5 mmol) in THF (200 mL) cooled at 0° C. was added LiAlH4 (1.0 M in THF, 111 mL, 111.2 mmol) dropwise. The reaction mixture was stirred at room temperature for 4 h and refluxed at 80° C. for 2 h, following which it was cooled to rt and carefully quenched with saturated aq. Na₂SO₄ solution. The resulting slurry was filtered through a pad of celite, the filtrate was washed with ethyl acetate and evaporated under reduced pressure. The crude product was purified by silica gel column chromatography to afford spiro[cyclopentane-1,3′-indoline 1.4 (10 g, 71%) as a yellowish powder. LCMS: 174.10 [M+H]⁺.

### Step-3; Procedure for synthesis of 1′-((1,1-dimethyl-1,3-dihydroisobenzofuran-5-yl)sulfonyl)spiro[cyclopentane-1,3′-indoline]

To a stirred solution of spiro[cyclopentane-1,3′-indoline 1.4 (100 mg, 0.57 mmol, 1 eq) in acetonitrile (10 mL) was added pyridine (0.09 mL, 1.1 mmol, 2 eq) at room temperature and stirred for 5 min, 1,1-dimethyl-1,3-dihydroisobenzofuran-5-sulfonyl chloride 4.1 (142 mg, 0.57 mmol, 1 eq) was added and the reaction mixture was stirred at room temperature for 2 h. The progress of the reaction was monitored by TLC. After completion of the reaction, the reaction mixture was diluted with water and extracted with ethyl acetate. The combined organic layers were dried over anhydrous Na₂SO₄, filtered and concentrated under reduced pressure. The crude product was purified by reverse phase preparative HPLC to afford 1′-((1,1-dimethyl-1,3-dihydroisobenzofuran-5-yl)sulfonyl)spiro[cyclopentane-1,3′-indoline] 1-66.

Yield: 25 mg, 11%; Appearance: Off-white solid; ¹H NMR (400 MHz, DMSO-d₆) δ 1.38 (s, 6H) 1.45-1.76 (m, 8H) 3.71 (s, 2H) 4.96 (s, 2H) 7.03 (d, J=7.34 Hz, 1H) 7.15-7.24 (m, 2H) 7.46 (d, J=8.31 Hz, 2H) 7.73 (d, J=7.83 Hz, 1H) 7.78 (s, 1H); HPLC purity: 99.59%; LCMS calculated for C₂₂H₂₅NO₃S: 383.16; Observed: 384.05 [M+H]⁺. Example 4(2)—Synthesis of 1′-((2,3-dihydro-1H-inden-5-yl)sulfonyl)spiro[cyclopentane-1,3′-indoline] (I-67)

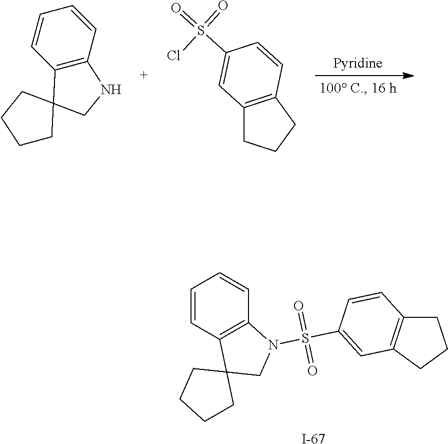

Sulfonyl chloride (1.1 eq.) was added to the vial containing aniline (1 eq.) in dry pyridine (1 mL). The reaction mixture was heated at 100° C. with stirring for 16 hours. After cooling to the room temperature, the mixture was evaporated. The residue was dissolved in DMSO (2 mL), filtered from non-soluble impurities if there were any. The resulting filtrate was subjected to HPLC purification (deionized water/HPLC-grade methanol (acetonitrile)).

Yield: 25.2 mg, 16.8%; Appearance: Pink solid; ¹H NMR (600 MHz, DMSO-d₆) δ 7.65 (s, 1H), 7.56 (dd, J=8.0, 1.8 Hz, 1H), 7.42 (d, J=8.1 Hz, 1H), 7.36 (d, J=7.9 Hz, 1H), 7.21-7.14 (m, 1H), 7.12 (d, J=7.5 Hz, 1H), 7.01-6.93 (m, 1H), 3.65 (s, 2H), 2.85 (t, J=7.5 Hz, 4H), 1.97 (p, J=7.5 Hz, 2H), 1.75-1.67 (m, 2H), 1.64 (dt, J=8.9, 6.4 Hz, 2H), 1.57 (dt, J=14.3, 7.0 Hz, 2H), 1.53-1.44 (m, 2H); HPLC purity: 100%; LCMS Calculated for C₂₁H₂₃NO₂S: 353.48; Observed: 354.2 [M+H]⁺.

### Example 5—Synthesis of 4-({1′,2′-dihydrospiro[cyclopentane-1,3′-indol]-1′-yl}sulfonyl)-N,N-dimethylbenzene-1-sulfonamide (I-68)

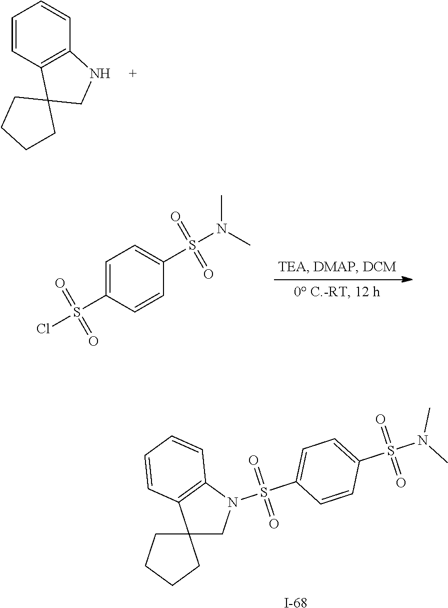

4-(dimethylsulfamoyl)benzene-1-sulfonyl chloride (0.429 g, 1.51 mmol) was added to an ice-cooled solution of 1′,2′-dihydrospiro[cyclopentane-1,3′-indole] (0.25 g, 1.44 mmol) and triethylamine (0.29 g, 2.86 mmol) in DCM (10 mL). After, DMAP (0.08 g, 0.654 mmol) was added and the reaction mixture was allowed to warm to room temperature and stir until completion (overnight, NMR control). After the reaction mixture was diluted with water (10 mL), the organic layer was separated, dried over MgSO₄ and concentrated in vacuo. The residue was subjected to HPLC purification (deionized water/HPLC-grade acetonitrile) that afforded 4-({1′,2′-dihydrospiro[cyclopentane-1,3′-indol]-1′-yl}sulfonyl)-N,N-dimethylbenzene-1-sulfonamide, I-68. Yield: 341.3 mg, 53.5%; Appearance: White solid; ¹H NMR (400 MHz, DMSO-d₆) δ 8.04 (d, J=8.6 Hz, 2H), 7.97-7.88 (m, 2H), 7.51 (d, J=8.1 Hz, 1H), 7.33-7.21 (m, 1H), 7.18 (d, J=7.5 Hz, 1H), 7.07 (t, J=7.4 Hz, 1H), 3.75 (s, 2H), 2.58 (s, 6H), 1.67 (d, J=7.9 Hz, 2H), 1.60 (dd, J=10.0, 6.0 Hz, 2H), 1.51 (p, J=6.2 Hz, 2H), 1.35 (dd, J=11.7, 6.3 Hz, 2H); HPLC purity: 100%; LCMS Calculated for C₂₀H₂₄N₂O₄S₂: 420.54; Observed: 421.2 [M+H]⁺. Example 6—Synthesis of 4-({1,2-dihydrospiro[indole-3,4′-oxan]-1-yl}sulfonyl)-N,N-dimethylbenzene-1-sulfonamide (I-69)

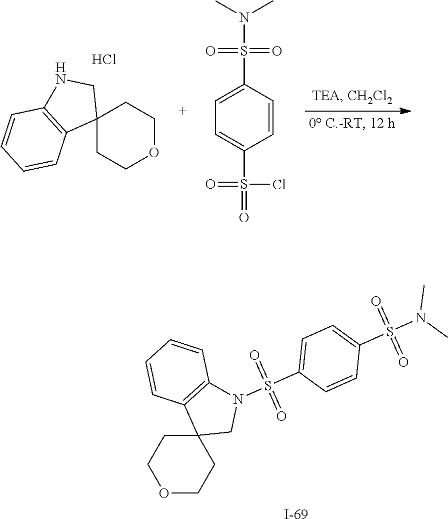

4-(dimethylsulfamoyl)benzene-1-sulfonyl chloride (0.125 g, 0.44 mmol) was added at 0° C. to a stirred solution of 1,2-dihydrospiro[indole-3,4′-oxane] hydrochloride (0.1 g, 0.44 mmol) and diisopropylethylamine (0.142 g, 1.1 mmol) in DCM (3 mL). The resulting mixture was stirred for 12 h at room temperature and poured into water (5 mL). The organic layer was separated, washed with 10% aq NaHSO₄ solution (5 mL), saturated aq NaHCO₃ solution (5 mL), brine (5 mL), dried over sodium sulfate, filtered and evaporated under reduced pressure. Crude residue was subjected to HPLC purification (deionized water/HPLC-acetonitrile, TFA) that afforded the 4-({1,2-dihydrospiro[indole-3,4′-oxan]-1-yl}sulfonyl)-N,N-dimethylbenzene-1-sulfonamide, I-69. Yield: 110.1 mg, 53.8%; Appearance: Yellow solid; ¹H NMR (400 MHz, DMSO-d₆) δ 8.07 (d, J=8.0 Hz, 2H), 7.91 (d, J=8.1 Hz, 2H), 7.52 (d, J=8.0 Hz, 1H), 7.34-7.19 (m, 2H), 7.08 (t, J=7.5 Hz, 1H), 3.97 (s, 2H), 3.79-3.62 (m, 2H), 3.41 (t, J=12.2 Hz, 2H), 2.58 (s, 6H), 1.71 (dt, J=14.2, 7.0 Hz, 2H), 0.97 (d, J=13.3 Hz, 2H); HPLC purity: 100%; LCMS Calculated for C₂₀H₂₄N₂O₅S₂: 436.54, Observed: 437.0 [M+H]⁺.

### Example 7—Synthesis of 4-({1′,2′-dihydrospiro[cyclopentane-1,3′-pyrrolo[3,2-c]pyridin]-1′-yl}sulfonyl)-N,N-dimethylbenzene-1-sulfonamide (I-70)

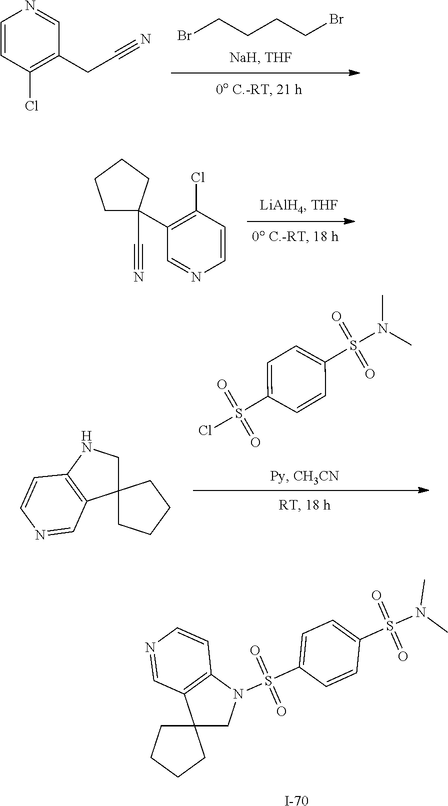

### Step 1. Synthesis of 1-(4-chloropyridin-3-yl)cyclopentane-1-carbonitrile

2-(4-chloropyridin-3-yl)acetonitrile (10 g, 65.5 mmol) was added to a slurry of sodium hydride (60% w/w; 7.83 g, 196 mmol) in anhydrous THF (300 mL) at room temperature over 30 min. The reaction mixture was stirred for 3 h and cooled to 0° C. 1,4-dibromobutane (14.1 g, 65.5 mmol) was added over 15 min. them the reaction mixture was allowed to warm up to room temperature and stir for 18 h. Then it was poured in water (300 mL) and extracted with ethyl acetate (200 mL×3). The organic layer was washed with water (500 mL) and brine (500 mL), dried over anhydrous sodium sulfate, filtered and concentrated under reduced pressure to afford 1-(4-chloropyridin-3-yl)cyclopentane-1-carbonitrile as beige oil (10.2 g, 49.3 mmol, 95% purity, 75.5% yield).

### Step 2. Synthesis of 1′,2′-dihydrospiro[cyclopentane-1,3′-pyrrolo[3,2-c]pyridine]

1-(4-chloropyridin-3-yl)cyclopentane-1-carbonitrile (2 g, 9.67 mmol) was added to a slurry of lithium aluminium hydride (0.550 g, 14.5 mmol) in anhydrous THF (150 mL) at 0° C. over 30 min and the reaction mixture was allowed to warm up and stir at room temperature for 18 h. Then, it was cooled to 0° C. and quenched with water (2 mL) and diluted with ethyl acetate (100 mL). The organic layer was separated, dried over anhydrous sodium sulfate, filtered and concentrated under reduced pressure to afford 1′,2′-dihydrospiro[cyclopentane-1,3′-pyrrolo[3,2-c]pyridine] (0.8 g, 4.59 mmol, 95% purity, 45.2% yield).

### Step 3. Synthesis of 4-({1′,2′-dihydrospiro[cyclopentane-1,3′-pyrrolo[3,2-c]pyridin]-1′-yl}sulfonyl)-N,N-dimethylbenzene-1-sulfonamide

Pyridine (0.271 g, 3.43 mmol) and 4-(dimethylsulfamoyl)benzene-1-sulfonyl chloride (0.649 g, 2.29 mmol) were added to a solution of 1′,2′-dihydrospiro[cyclopentane-1,3′-pyrrolo[3,2-c]pyridine] (0.4 g, 2.29 mmol) in acetonitrile (25 mL). The reaction mixture was stirred at room temperature for 18 h until completion (TLC control) and the solvent was removed under reduced pressure. The residue was subjected to HPLC purification (deionized water/HPLC-grade acetonitrile) that afforded the 4-({1′,2′-dihydrospiro[cyclopentane-1,3′-pyrrolo[3,2-c]pyridin]-1′-yl}sulfonyl)-N,N-dimethylbenzene-1-sulfonamide, 1-70. Yield: 85.9 mg, 8.45%; Appearance: Beige solid; ¹H NMR (400 MHz, DMSO-d₆) δ 8.50-8.32 (m, 2H), 8.14 (d, J=8.0 Hz, 2H), 7.96 (d, J=8.2 Hz, 2H), 7.44 (d, J=5.5 Hz, 1H), 3.81 (s, 2H), 2.61 (s, 6H), 1.70 (d, J=32.7 Hz, 6H), 1.55 (s, 2H); HPLC purity: 96.07%; LCMS Calculated for C₁₉H₂₃N₃O₄S₂: 421.53; Observed: 422.0 [M+H]⁺.

### Example 8—Synthesis of 4-({1′,2′-dihydrospiro[cyclopentane-1,3′-pyrrolo[2,3-b]pyridin]-1′-yl}sulfonyl)-N,N-dimethylbenzene-1-sulfonamide (I-71)

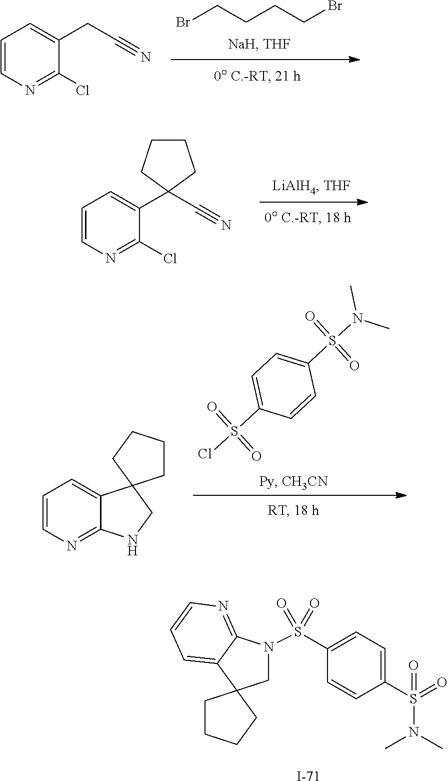

### Step 1. Synthesis of 1-(2-chloropyridin-3-yl)cyclopentane-1-carbonitrile

2-(2-chloropyridin-3-yl)acetonitrile (2 g, 13.1 mmol) was added to a slurry of sodium hydride (60% w/w; 0.314 g, 13.1 mmol) in anhydrous THF (50 m L) at room temperature over 30 min. The reaction mixture was stirred for 3 h and cooled to 0° C. 1,4-dibromobutane (2.82 g, 13.1 mmol) was added over 15 min to it. Then the reaction mixture was allowed to warm up to room temperature and stir for 18 h. After it was poured in water (50 mL) and extracted with ethyl acetate (50 mL×3). The organic layer was washed with water (50 mL) and brine (50 mL), dried over anhydrous sodium sulfate, filtered and concentrated under reduced pressure to afford 1-(2-chloropyridin-3-yl)cyclopentane-1-carbonitrile (2.69 g, 13 mmol, 95% purity, 94.4% yield).

### Step 2. Synthesis of 1′,2′-dihydrospiro[cyclopentane-1,3′-pyrrolo[2,3-b]pyridine]

1-(2-chloropyridin-3-yl)cyclopentane-1-carbonitrile (1 g, 4.83 mmol) was added to a slurry of lithium aluminium hydride (0.201 g, 5.31 mmol) in anhydrous THF (30 mL) at 0° C. over 30 min and the reaction mixture was allowed to warm up and stir at room temperature for 18 h. Then, it was cooled to 0° C. and quenched with water (5 mL) and diluted with ethyl acetate (50 mL). The organic layer was separated, dried over anhydrous sodium sulfate, filtered and concentrated under reduced pressure to afford 1′,2′-dihydrospiro[cyclopentane-1,3′-pyrrolo[2,3-b]pyridine] (0.5 g, 2.86 mmol, 85% purity, 50.5% yield) that was used in next step without further purification.

### Step 3. Synthesis of 4-({1′,2′-dihydrospiro[cyclopentane-1,3′-pyrrolo[2,3-b]pyridin]-1′-yl}sulfonyl)-N,N-dimethylbenzene-1-sulfonamide

Pyridine (2.26 g, 28.6 mmol) and 4-(dimethylsulfamoyl)benzene-1-sulfonyl chloride (0.89 g, 3.14 mmol) were added to a solution of 1′,2′-dihydrospiro[cyclopentane-1,3′-pyrrolo[2,3-b]pyridine] (0.5 g, 2.86 mmol) in acetonitrile (5 mL). The solution was stirred overnight and evaporated. The residue was subjected to HPLC purification (deionized water/HPLC-grade acetonitrile) that afforded 4-({1′,2′-dihydrospiro[cyclopentane-1,3′-pyrrolo[2,3-b]pyridin]-1′-yl}sulfonyl)-N,N-dimethylbenzene-1-sulfonamide, I-71. Yield: 100.0 mg, 7.98%, Appearance: Yellow solid; ¹H NMR (400 MHz, DMSO-d₆) δ 8.34-8.16 (m, 2H), 8.07 (dq, J=5.2, 1.7 Hz, 1H), 7.94 (dq, J=8.4, 1.6 Hz, 2H), 7.61 (dt, J=7.5, 1.7 Hz, 1H), 7.00 (tq, J=5.3, 1.6 Hz, 1H), 4.01-3.74 (m, 2H), 2.63 (q, J=1.6 Hz, 6H), 1.76 (d, J=12.8 Hz, 8H); H PLC purity: 100%; LCMS Calculated for C₁₉H₂₃N₃O₄S₂: 421.53; Observed: 422.2 [M+H]⁺.

### Example 9—Synthesis of 4-({4-hydroxy-1′,2′-dihydrospiro[cyclohexane-1,3′-indol]-1′-yl}sulfonyl)-N,N-dimethylbenzene-1-sulfonamide. (I-72)

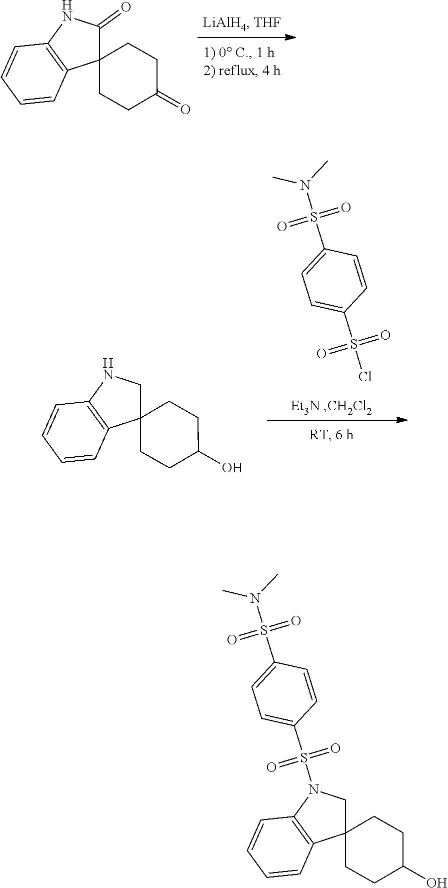 [SMILES: O=C1CCC2(CC1)C(=O)Nc1ccccc12 ; OC1CCC2(CC1)CNc1ccccc12 ; CN(C)S(=O)(=O)c1ccc(S(=O)(=O)Cl)cc1 ; CN(C)S(=O)(=O)c1ccc(S(=O)(=O)N2CC3(CCC(O)CC3)c3ccccc32)cc1]

### Step 1. Synthesis of 1′,2′-dihydrospiro[cyclohexane-1,3′-indol]-4-ol

A solution of 1′,2′-dihydrospiro[cyclohexane-1,3′-indole]-2′,4-dione (1 g, 4.64 mmol) in anhydrous THF (10 mL) was added to a solution of LiAlH4 (0.44 g, 11.57 mmol) in anhydrous THF (15 mL) at 0° C. dropwise for 1 hour. Then the reaction mixture was refluxed for 4 hours, cooled to room temperature and quenched with water (1.76 mL). The resulting mixture was filtered and the filter cake was washed with a mixture of DCM and MeOH (1/1 (v/v), 20 mL). The filtrate was concentrated in vacuum to give 1′,2′-dihydrospiro[cyclohexane-1,3′-indol]-4-ol (0.6 g, 2.95 mmol, 90% purity, 57.2% yield).

### Step 2. Synthesis of 4-({4-hydroxy-1′,2′-dihydrospiro[cyclohexane-1,3′-indol]-1′-yl}sulfonyl)-N,N-dimethylbenzene-1-sulfonamide

4-(dimethylsulfamoyl)benzene-1-sulfonyl chloride (0.278 g, 0.983 mmol) was added to a solution of 1′,2′-dihydrospiro[cyclohexane-1,3′-indol]-4-ol (0.2 g, 0.983 mmol) and pyridine (0.621 g, 7.86 mmol) in DCM (5 mL) and the reaction mixture was stirred for 6 h. After it was diluted with water (5 mL) and the product extracted with DCM (5 mL×2). The combined organic layers were washed with brine (10 mL), dried over MgSO4, filtered and concentrated under reduced pressure. The residue was subjected to HPLC purification (deionized water/HPLC-grade acetonitrile) to give 4-({4-hydroxy-1′,2′-dihydrospiro[cyclohexane-1,3′-indol]-1′-yl}sulfonyl)-N,N-dimethylbenzene-1-sulfonamide, I-72. Yield: 65.3 mg, 14.16%; Appearance: White solid; ¹H NMR (500 MHz, DMSO-d₆) δ 8.10-8.02 (m, 2H), 7.91 (dt, J=11.6, 3.5 Hz, 2H), 7.52 (d, J=7.9 Hz, 1H), 7.26 (t, J=8.0 Hz, 1H), 7.16 (d, J=7.7 Hz, 1H), 7.07 (q, J=7.8 Hz, 1H), 3.81 (s, 2H), 3.47-3.33 (m, 1H), 2.58 (d, J=1.8 Hz, 6H), 1.85-1.54 (m, 2H), 1.46 (d, J=14.0 Hz, 3H), 1.31-0.48 (m, 3H); HPLC purity: 75.86%; LCMS Calculated for C₂₁H₂₆N₂O₅S₂:450.57; Observed: 451.2 [M+H]⁺.

### Example 10—Synthesis of 4-{[1′-(2,2-dimethylpropanoyl)-1,2-dihydrospiro[indole-3,3′-pyrrolidin]-1-yl]sulfonyl}-N,N-dimethylbenzene-1-sulfonamide (I-73)

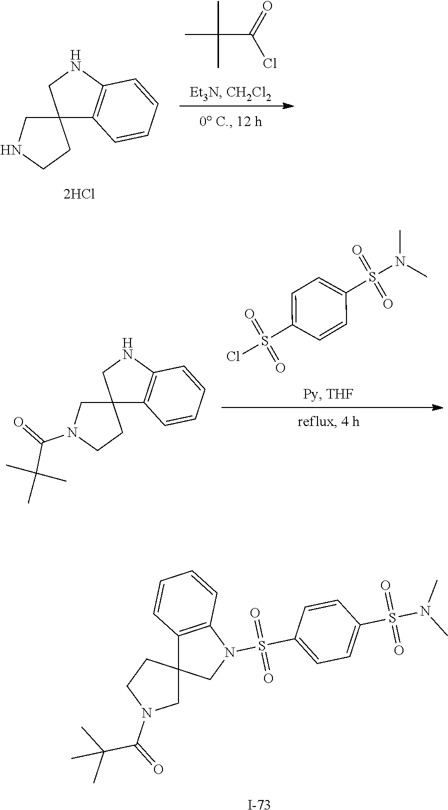

### Step 1. Synthesis of 1-{1,2-dihydrospiro[indole-3,3′-pyrrolidin]-1′-yl}-2,2-dimethylpropan-1-one

1,2-dihydrospiro[indole-3,3′-pyrrolidine] dihydrochloride (0.5 g, 2.02 mmol) and triethylamine (0.714 g, 7.06 mmol) were dissolved in dichloromethane (5 mL), the mixture was cooled to 0° C. and 2,2-dimethylpropanoyl chloride (0.243 g, 2.02 mmol) was added to it dropwise. The mixture was stirred overnight at room temperature and then washed with water (5 mL), brine (5 mL). The organic layer was dried over Na₂SO₄, filtered and the filtrate was evaporated under reduced pressure to afford 1-{1,2-dihydrospiro[indole-3,3′-pyrrolidin]-1′-yl}-2,2-dimethylpropan-1-one (0.6 g, 2.32 mmol, 79.55% purity, 91.5% yield) that was used in next step without further purification.

### Step 2. Synthesis of 4-{[1′-(2,2-dimethylpropanoyl)-1,2-dihydrospiro[indole-3,3′-pyrrolidin]-1-yl]sulfonyl}-N,N-dimethylbenzene-1-sulfonamide

4-(dimethylsulfamoyl)benzene-1-sulfonyl chloride (0.65 g, 2.29 mmol) was added to the mixture of crude 1-{1,2-dihydrospiro[indole-3,3′-pyrrolidin]-1′-yl}-2,2-dimethylpropan-1-one (0.6 g, 1.84 mmol, 79.55% purity) and pyridine (0.25 g, 3.16 mmol) in dry THE (20 mL). The reaction mixture was refluxed for 4 h and evaporated under reduced pressure. The crude material was purified by HPLC (deionized water/HPLC-grade methanol) to give 4-{[1′-(2,2-dimethylpropanoyl)-1,2-dihydrospiro[indole-3,3′-pyrrolidin]-1-yl]sulfonyl}-N,N-dimethy-lbenzene-1-sulfonamide, 1-73. Yield: 25.0 mg, 2.54%; Appearance: Pink solid; ¹H NMR (600 MHz, DMSO-d₆) δ 8.11-7.97 (m, 2H), 7.95-7.84 (m, 2H), 7.53 (d, J=8.2 Hz, 1H), 7.33-7.26 (m, 1H), 7.16 (s, 1H), 7.08 (td, J=7.6, 1.0 Hz, 1H), 3.90 (q, 0.1=11.3 Hz, 2H), 3.34 (s, 4H), 2.59 (s, 6H), 1.71 (d, J=66.5 Hz, 2H), 1.06 (s, 9H); HPLC purity: 100%; LCMS Calculated for C₂₄H₃₁N₃O₅S₂: 505.65; Observed: 506.2 [M+H]⁺.

### Example 11—Synthesis of 1′-(3-methylbenzenesulfonyl)-1′,2′-dihydrospiro[cyclopentane-1,3′-indole] (I-74)

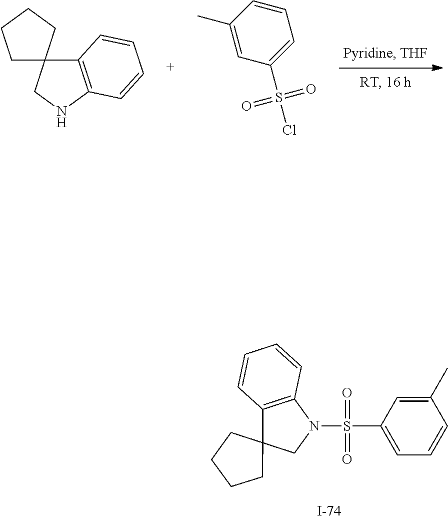

3-methylbenzene-1-sulfonyl chloride (0.5 g, 2.62 mmol) was added to the mixture of 1′,2′-dihydrospiro[cyclopentane-1,3′-indole] (0.453 g, 2.62 mmol) and pyridine (0.310 g, 3.93 mmol) in dry THF (20 mL). The reaction mixture was stirred overnight and evaporated. The residue was subjected to HPLC purification (deionized water/HPLC-grade methanol, ammonia) that afforded 1′-(3-methylbenzenesulfonyl)-1′,2′-dihydrospiro[cyclopentane-1,3′-indole], 1-74. Yield: 92.5 mg, 10.2%; Appearance: Pink solid; ¹H NMR (600 MHz, DMSO-d₆) δ 7.63 (s, 1H), 7.56 (d, J=7.8 Hz, 1H), 7.43 (dt, J=18.8, 7.9 Hz, 3H), 7.24-7.15 (m, 1H), 7.13 (dd, J=7.6, 1.2 Hz, 1H), 7.01-6.94 (m, 1H), 3.67 (s, 2H), 2.31 (s, 3H), 1.81-1.66 (m, 2H), 1.62 (dddd, J=19.2, 14.6, 9.9, 5.3 Hz, 2H), 1.58-1.51 (m, 2H), 1.50-1.33 (m, 2H); HPLC purity: 100%; LCMS Calculated for C₁₉H₂₁NO₂S: 327.44; Observed: 328.0 [M+H]⁺.

### Example 12—Synthesis of 1′-{[1-(3-methylphenyl)-1H-pyrazol-4-yl]sulfonyl}-1′,2′-dihydrospiro[cyclopentane-1,3′-indole] (I-75)

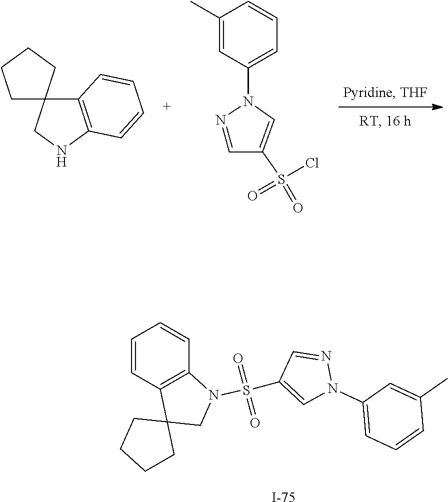

1-(3-methylphenyl)-1H-pyrazole-4-sulfonyl chloride (0.5 g, 1.94 mmol) was added to the mixture of 1′,2′-dihydrospiro[cyclopentane-1,3′-indole] (0.34 g, 1.96 mmol) and pyridine (0.23 g, 2.9 mmol) in dry THF (20 mL). The reaction mixture was stirred overnight at room temperature and evaporated. The residue was subjected to HPLC purification (deionized water/HPLC-grade methanol, ammonia) that afforded 1′-{[1-(3-methylphenyl)-1H-pyrazol-4-yl]sulfonyl}-1′,2′-dihydrospiro[cyclopentane-1,3′-indole], I-75. Yield: 64.0 mg, 8.0%; Appearance: Yellow solid; ¹H NMR (400 MHz, DMSO-d₆) δ 9.23 (s, 1H), 8.13 (s, 1H), 7.72 (s, 1H), 7.65 (d, J=8.1 Hz, 1H), 7.49 (d, J=8.0 Hz, 1H), 7.39 (t, J=7.9 Hz, 1H), 7.20 (q, J=7.0 Hz, 3H), 7.02 (t, J=7.5 Hz, 1H), 3.73 (s, 2H), 2.36 (s, 3H), 1.89-1.56 (m, 8H); HPLC purity: 100%; LCMS Calculated for C₂₂H₂₃N₃O₂S: 393.51; Observed: 394.2 [M+H]⁺.

### Example 13—Synthesis of 4-({1,2-dihydrospiro[indole-3,3′-oxolan]-1-yl}sulfonyl)-N,N-dimethylbenzene-1-sulfonamide (I-76)

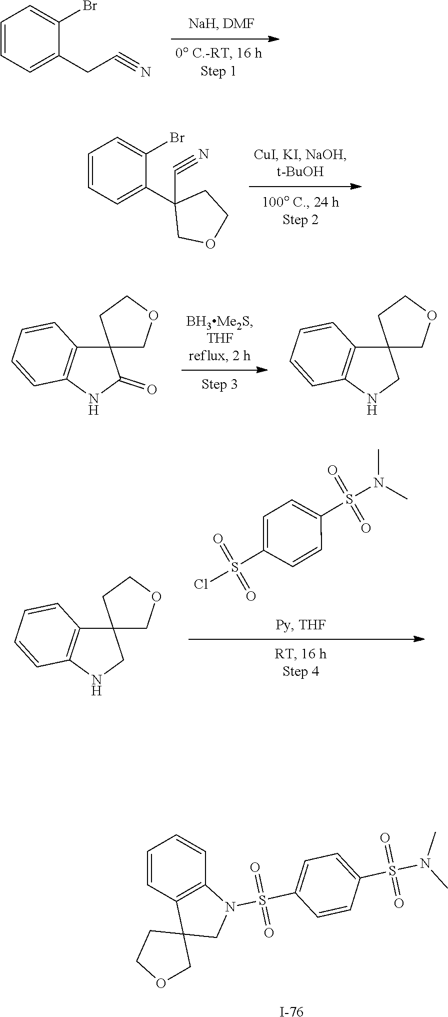

### Step 1. Synthesis of 3-(2-bromophenyl)oxolane-3-carbonitrile

2-(2-bromophenyl)acetonitrile (3 g, 15.3 mmol) was added portionwise to the suspension of sodium hydride (1.83 g, 45.9 mmol, 60 w %) in DMF (20 mL) at 0° C. The mixture was stirred 30 min at this temperature and 1-bromo-2-(chloromethoxy)ethane (2.65 g, 15.3 mmol)) was added portionwise at the same temperature. After the mixture was allowed to warm up to room temperature and stir overnight until completion. The solvent was evaporated under reduced pressure, the residue was treated with water/ethyl acetate mixture (30 mL/30 mL). The organic layer was separated, dried over sodium sulfate, filtered and evaporated under reduced pressure. The crude product was purified by flash chromatography (hexane/MTBE) to afford 3-(2-bromophenyl)oxolane-3-carbonitrile (1.6 g, 6.34 mmol, 95% purity, 39.4% yield).

### Step 2. Synthesis of 1,2-dihydrospiro[indole-3,3′-oxolan]-2-one

Potassium iodide (0.105 g, 0.634 mmol), copper iodide (0.120 g, 0.634 mmol), and N-acetylglycine (0.0742 g, 0.634 mmol) were added to a solution of 3-(2-bromophenyl)oxolane-3-carbonitrile (1.6 g, 6.34 mmol) and sodium hydroxide (0.76 g, 19 mmol) in tert-butanol (20 mL). The mixture was refluxed for 24 h, then cooled to room temperature, filtered through silica, and the filtrate was evaporated to dryness. The residue was treated with water/ethyl acetate mixture (50 mL/50 mL). The organic layer was separated, dried over sodium sulfate, filtered and evaporated under reduced pressure. The crude product was purified by flash chromatography (chloroform/MTBE) to afford 1,2-dihydrospiro[indole-3,3′-oxolan]-2-one (0.4 g, 2.11 mmol, 95% purity, 31.9% yield).

### Step 3. Synthesis of 1,2-dihydrospiro[indole-3,3′-oxolane]

10 M dimethylsulfide borane complex solution in THF (0.63 mL, 0.480 g, 6.32 mmol) was added to a solution of 1,2-dihydrospiro[indole-3,3′-oxolan]-2-one (0.4 g, 2.11 mmol) in dry THF (20 mL), the mixture was refluxed for 2 h and cooled to room temperature. Then methanol (10 mL) was added dropwise, the mixture was refluxed for 2 h, cooled to room temperature and evaporated under reduced pressure. The residue was treated with water/ethyl acetate mixture (20 mL/20 mL). The organic layer was separated, dried over sodium sulfate, filtered and evaporated under reduced pressure to afford crude 1,2-dihydrospiro[indole-3,3′-oxolane] (0.3 g, 1.71 mmol, 46.6% purity, 37.6% yield) that was used in the next step without further purification.

### Step 4. Synthesis of 4-({1,2-dihydrospiro[indole-3,3′-oxolan]-1-yl}sulfonyl)-N,N-dimethylbenzene-1-sulfonamide

4-(dimethylsulfamoyl)benzene-1-sulfonyl chloride (0.485 g, 1.71 mmol) was added to the mixture of 1,2-dihydrospiro[indole-3,3′-oxolane] (0.3 g, 1.71 mmol) and pyridine (0.202 g, 2.56 mmol) in dry THF (20 mL). The reaction mixture was stirred overnight and evaporated to dryness. The residue was subjected to HPLC purification (deionized water/HPLC-grade methanol) that afforded 4-({1,2-dihydrospiro[indole-3,3′-oxolan]-1-yl}sulfonyl)-N,N-dimethylbenzene-1-sulfonamide, I-76. Yield: 133.7 mg, 17.5%; Appearance: Yellow solid; ¹H NMR (400 MHz, DMSO-d₆) δ 8.04 (dd, J=8.4, 1.6 Hz, 2H), 7.93 (dd, J=8.4, 1.6 Hz, 2H), 7.54 (d, J=8.1 Hz, 1H), 7.31 (t, J=8.0 Hz, 1H), 7.26 (d, J=7.5 Hz, 1H), 7.11 (t, J=7.5 Hz, 1H), 3.93 (s, 2H), 3.88 (dd, J=8.5, 4.7 Hz, 1H), 3.80 (q, 1=7.8 Hz, 1H), 2.60 (d, J=1.6 Hz, 6H), 1.97-1.89 (m, 1H), 1.83 (dq, J=12.5, 6.6, 6.0 Hz, 1H); HPLC purity: 100%; LCMS Calculated for C₉H₂₂N₂O₅S₂: 422.52; Observed: 423.0 [M+H]⁺.

### Example 14—Synthesis of 4-({1,2-dihydrospiro[indole-3,2′-oxolan]-1-yl}sulfonyl)-N,N-dimethylbenzene-1-sulfonamide (I-77)

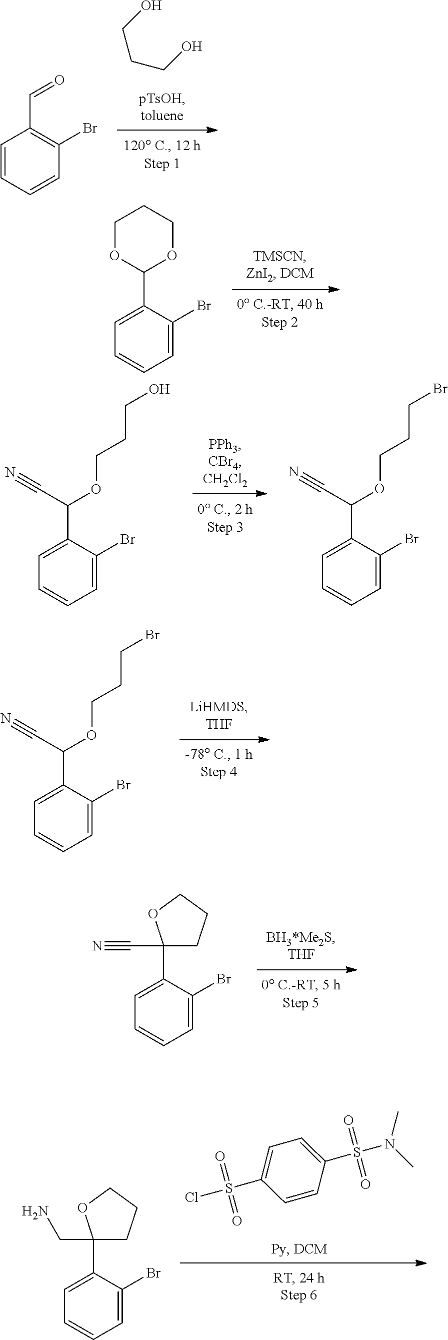

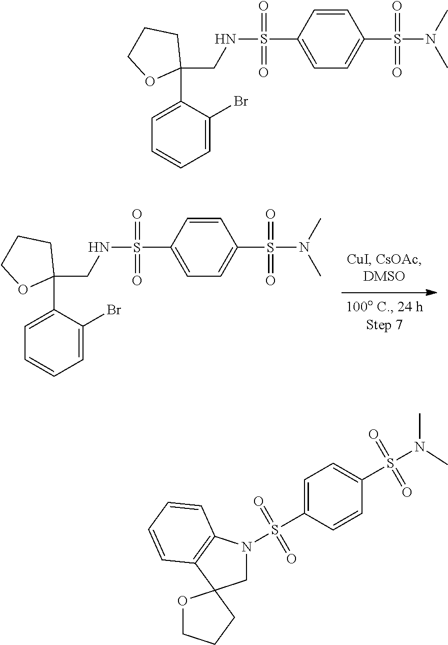 [SMILES: CN(C)S(=O)(=O)c1ccc(S(=O)(=O)NCC2(c3ccccc3Br)CCCO2)cc1 ; CN(C)S(=O)(=O)c1ccc(S(=O)(=O)NCC2(c3ccccc3Br)CCCO2)cc1 ; CN(C)S(=O)(=O)c1ccc(S(=O)(=O)N2CC3(CCCO3)c3ccccc32)cc1]

### Step 1. Synthesis of 2-(2-bromophenyl)-1,3-dioxane

Propane-1,3-diol (2.26 g, 29.7 mmol) and p-TsOH (0.464 g, 2.7 mmol) were added to a solution of 2-bromobenzaldehyde (5 g, 27 mmol) in toluene (50 mL). The mixture was stirred at 120° C. with Dean-Stark apparatus until complete water removal from reaction mixture (overnight). After cooling to room temperature, the reaction mixture was washed with brine (50 mL) and dried over magnesium sulfate. The evaporation under reduced pressure afforded 2-(2-bromophenyl)-1,3-dioxane (6 g, 24.6 mmol, 100/6 purity, 91.4% yield).

### Step 2. Synthesis of 2-(2-bromophenyl)-2-(3-hydroxypropoxy)acetonitrile

Zinc iodide (1.96 g, 6.15 mmol) was added to an ice-cold solution of 2-(2-bromophenyl)-1,3-dioxane (3 g, 12.3 mmol) in DCM (30 mL) followed by addition of trimethylsilyl cyanide (1.82 g, 18.4 mmol) over a period of 10 minutes keeping the temperature at 0° C. After cooling was removed and the reaction was stirred for 40 hours at room temperature until the completion (TLC control). The mixture was poured into water (60 mL) and extracted with DCM (60 mL×3), the combined organic layers were washed with brine (80 mL), dried over magnesium sulfate and concentrated in vacuo. The purification of residue by column chromatography (chloroform/acetonitrile) afforded 2-(2-bromophenyl)-2-(3-hydroxypropoxy)acetonitrile (0.5 g, 1.85 mmol, 95% purity, 14.3% yield).

### Step 3. Synthesis of 2-(2-bromophenyl)-2-(3-bromopropoxy)acetonitrile

A solution of 2-(2-bromophenyl)-2-(3-hydroxypropoxy)acetonitrile (0.5 g, 1.85 mmol) and tetrabromomethane (1.71 g, 5.18 mmol) in DCM (30 mL) was cooled to 0° C. and the solution of triphenylphosphine (1.35 g, 5.18 mmol) in DCM (10 mL) was added dropwise over a period of 5 minutes. The reaction was stirred for 2 hours at 0° C. and then allowed to warm to room temperature. Ether (50 mL) was added to the mixture and the cloudy solution was filtered through a plug of celite. The filtrate was concentrated in vacuo and purified by flash chromatography (MTBE/hexane) to yield 2-(2-bromophenyl)-2-(3-bromopropoxy)acetonitrile (0.55 g, 1.65 mmol, 95% purity, 84.7% yield).

### Step 4. Synthesis of 2-(2-bromophenyl)oxolane-2-carbonitrile

A solution of 2-(2-bromophenyl)-2-(3-bromopropoxy)acetonitrile (0.55 g, 1.65 mmol) in THF (60 mL) was cooled to −78° C. followed by addition of lithium bis(trimethylsilyl)amide (0.441 g, 2.64 mmol) 1M solution in THF (2.7 mL) and the resulting mixture was stirred for 1 hour at −78° C. After sat. aq. NH₄C₁ solution (10 mL) was added and the mixture was allowed to warm to room temperature. The reaction mixture was diluted with water (10 mL) and the product was extracted with ethyl acetate (15 mL×3). The combined organic layers were washed with brine (30 mL), dried over magnesium sulfate, filtered and concentrated in vacuo to give 2-(2-bromophenyl)oxolane-2-carbonitrile (0.3 g, 1.18 mmol, 95% purity, 72.2% yield).

### Step 5. Synthesis of 1-[2-(2-bromophenyl)oxolan-2-yl]methanamine

A solution of 2-(2-bromophenyl)oxolane-2-carbonitrile (0.3 g, 1.22 mmol) in THF (20 mL) was cooled to 0° C. followed by addition of BH₃ dimethyl sulfide complex (0.268 g, 3.54 mmol) solution in THF under argon atmosphere. The resulting mixture was stirred at room temperature for 5 h. Then the mixture was cooled to 0° C. and methanol (10 mL) was added to quench the reaction. The resulting mixture was stirred under reflux conditions for 0.5 h. Following concentration under reduced pressure afforded 1-[2-(2-bromophenyl)oxolan-2-yl]methanamine (0.04 g, 0.156 mmol, 95.78% purity, 12.8% yield).

### Step 6. Synthesis of N4-{[2-(2-bromophenyl)oxolan-2-yl]methyl}-N1,N1-dimethylbenzene-1,4-disulfonamide

4-(dimethylsulfamoyl)benzene-1-sulfonyl chloride (0.05 g, 0.176 mmol) was added to a solution of 1-[2-(2-bromophenyl)oxolan-2-yl]methanamine (0.04 g, 0.156 mmol) and pyridine (0.037 g, 0.468 mmol) in DCM (5 mL). The reaction mixture was stirred at room temperature for 24 h. Then NH₄Cl sat. aq. solution (10 mL) was added to the reaction mixture and the product was extracted with DCM (10 mL×2). Combined organic layers were dried over magnesium sulfate, filtered and concentrated in vacuum to give N4-{[2-(2-bromophenyl)oxolan-2-yl]methyl}-N1,N1-dimethylbenzene-1,4-disulfonamide (0.1 g, 0.168 mmol, 88% purity, 95.2% yield) that was used in next step without further purification.

### Step 7. Synthesis of 4-{[(3R)-1,2-dihydrospiro[indole-3,2′-oxolan]-1-yl]sulfonyl}-N,N-dimethylbenzene-1-sulfonamide

A mixture of CuI (0.0754 g, 0.396 mmol), CsOAc (0.264 g, 1.38 mmol) and N4-{[2-(2-bromophenyl)oxolan-2-yl]methyl}-N1,N1-dimethylbenzene-1,4-disulfonamide (0.100 g, 0.198 mmol) in a 10 mL round-bottom flask was dried under high-vacuum for 0.5 h. After the flask was purged with argon, anhydrous DMSO (2.0 mL) was added and the mixture was heated at 100° C. for 24 h under argon atmosphere. After the mixture was diluted with ethyl acetate (30 mL) and sequentially washed with NH₄Cl sat. aq. solution (10 mL) and 1 M aq HCl solution (10 mL), brine (20 mL). The organic layer was dried over magnesium sulfate and concentrated to afford crude product which was subjected to HPLC purification (deionized water/HPLC-grade methanol) to afford 4-{[1,2-dihydrospiro[indole-3,2′-oxolan]-1-yl]sulfonyl}-N,N-dimethylbenzene-1-sulfonamide, I-77. Yield: 32.8 mg, 39.2%; Appearance: Yellow solid; ¹H NMR (400 MHz, DMSO-d₆) δ 8.07-7.98 (m, 2H), 7.90 (dd, J=8.6, 1.8 Hz, 2H), 7.56 (d, J=8.1 Hz, 1H), 7.45-7.23 (m, 2H), 7.13 (t, J=7.4 Hz, 1H), 3.96-3.80 (m, 2H), 3.72 (d, J=6.3 Hz, 2H), 2.60 (d, J=1.7 Hz, 6H), 2.12-1.82 (m, 4H). HPLC purity: 95.43%; LCMS Calculated for C₁₉H₂₂N₂O₅S₂: 422.52, Observed: 423.0 [M−H]⁺.

### Example 15—Synthesis of N,N-dimethyl-4-(spiro[cyclopentane-1,3′-pyrrolo[3,2-b]pyridin]-1′(2′H)-ylsulfonyl)benzenesulfonamide (I-78)

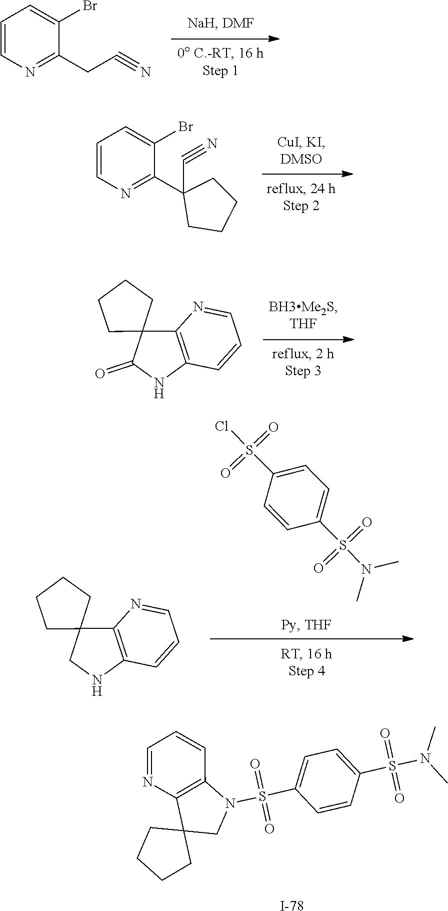

### Step 1. Synthesis of 1-(3-bromopyridin-2-yl)cyclopentanecarbonitrile

2-(3-bromopyridin-2-yl)acetonitrile (2 g, 10.1 mmol) was added dropwise to the suspension of sodium hydride (60 w %, 1.21 g, 30.3 mmol) in DMF (20 mL) at 0° C. The mixture was stirred for 30 min at this temperature and 1,4-dibromobutane (2.18 g, 10.1 mmol) was slowly added. The solution was allowed to warm up to room temperature and stir overnight. After, the solvent was evaporated under reduced pressure, the residue was treated with mixture water (30 mL)/ethyl acetate (30 mL). The organic layer was separated, the water layer was extracted with ethyl acetate (30 mL×2). The combined organic layers were dried over sodium sulfate, filtered and evaporated under reduced pressure. The residue was purified by column chromatography (hexane/MTBE) to afford 1-(3-bromopyridin-2-yl)cyclopentanecarbonitrile (1.2 g, 4.77 mmol, 95% purity, 45.0% yield).

### Step 2. Synthesis of 1′,2′-dihydrospiro[cyclopentane-1,3′-pyrrolo[3,2-b]pyridin]-2′-one

Potassium iodide (0.0791 g, 0.477 mmol), copper iodide (0.0908 g, 0.477 mmol), and 2-acetamidoacetic acid (0.0558 g, 0.477 mmol) were added to the solution of 1-(3-bromopyridin-2-yl)cyclopentane-1-carbonitrile (1.2 g, 4.77 mmol)) followed by NaOH (0.571 g, 14.3 mmol) solution in t-BuOH (20 mL). The mixture was refluxed for 24 h, then filtered through silica, evaporated under reduced pressure. Water (30 mL) was added to the residue, and the product was extracted with ethyl acetate (30 mL×3). Combined organic layers were dried over sodium sulfate, filtered and evaporated to afford crude product that was purified by column chromatography (chloroform/ethyl acetate) to give 1′,2′-dihydrospiro[cyclopentane-1,3′-pyrrolo[3,2-b]pyridin]-2′-one (0.5 g, 2.65 mmol, 95% purity, 52.9% yield).

### Step 3. Synthesis of 1′,2′-dihydrospiro[cyclopentane-1,3′-pyrrolo[3,2-b]pyridine]

Dimethylsulfide borane complex (0.603 g, 7.95 mmol, 0.795 mL of 10 M in THF solution) was added to the solution of 1′,2′-dihydrospiro[cyclopentane-1,3′-pyrrolo[3,2-b]pyridin]-2′-one (0.5 g, 2.65 mmol) in dry THF (20 mL). The reaction mixture was refluxed until completion (TLC control, 2 h). Then methanol (10 mL) was added dropwise, the solvent was evaporated, water (20 mL) was added and the product was extracted with ethyl acetate (20 mL×3). After flash chromatography (chloroform-ethyl acetate) 1′,2′-dihydrospiro[cyclopentane-1,3′-pyrrolo[3,2-b]pyridine] (0.3 g, 1.72 mmol, 95% purity, 65% yield) was obtained.

### Step 4. Synthesis of N,N-dimethyl-4-(spiro[cyclopentane-1,3′-pyrrolo[3,2-b]pyridin]-1′(2′H)-ylsulfonyl)benzenesulfonamide

4-(dimethylsulfamoyl)benzene-1-sulfonyl chloride (0.488 g, 1.72 mmol) was added to the mixture of 1′,2′-dihydrospiro[cyclopentane-1,3′-pyrrolo[3,2-b]pyridine] (0.3 g, 1.72 mmol) and pyridine (0.203 g, 2.57 mmol) in dry THF (20 mL). The reaction mixture was stirred overnight and evaporated. The residue was subjected to HPLC purification (deionized water/HPLC-grade methanol) that afforded N,N-dimethyl-4-(spiro[cyclopentane-1,3′-pyrrolo[3,2-b]pyridin]-1′(2′H)-ylsulfonyl)benzenesulfonamide, 1-78. Yield: 168.4 mg, 21.9%; Appearance: Beige solid; ¹H NMR (400 MHz, DMSO-d₆) δ 8.21 (dd, 1=4.9, 1.4 Hz, 1H), 8.07 (d, J=8.3 Hz, 2H), 7.94 (d, J=8.3 Hz, 2H), 7.81 (dd, J=8.1, 1.4 Hz, 1H), 7.30-7.20 (m, 1H), 3.84 (s, 2H), 2.59 (d, J=1.8 Hz, 6H), 1.79-1.55 (m, 6H), 1.37 (s, 2H). HPLC purity: 100%; LCMS Calculated for C₁₉H₂₃N₃O₄S₂: 421.53; Observed: 422.2 [M−H]⁺.

### Example 16—Synthesis of N,N-dimethyl-4-((6′-methylspiro[cyclopentane-1,3′-indolin]-1′-yl)sulfonyl)benzenesulfonamide (I-79)

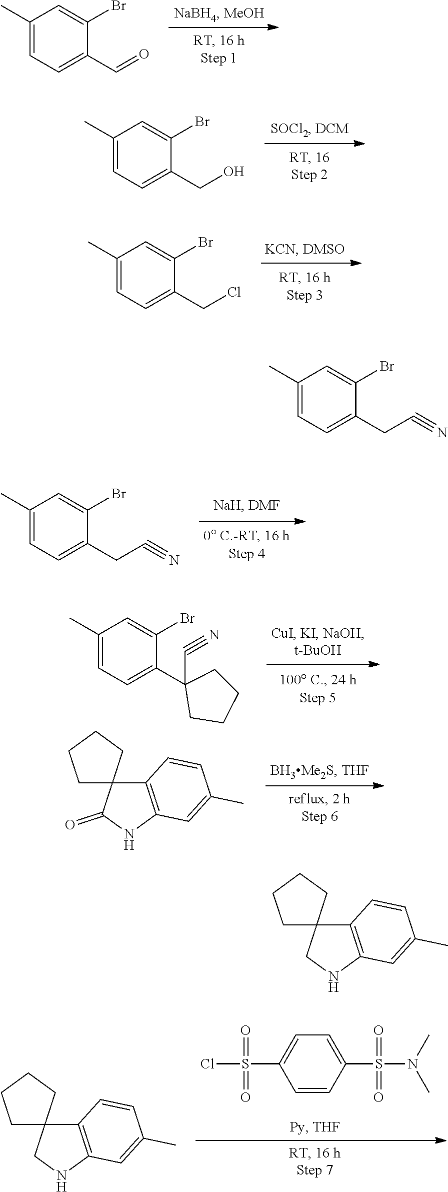

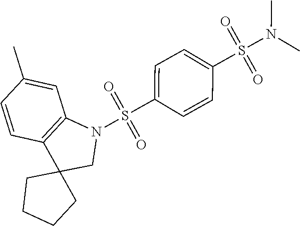 [SMILES: Cc1ccc2c(c1)N(S(=O)(=O)c1ccc(S(=O)(=O)N(C)C)cc1)CC21CCCC1]

### Step 1. Synthesis of (2-bromo-4-methylphenyl)methanol

Sodium borohydride (0.283 g, 7.45 mmol) was added portionwise to a solution of 2-bromo-4-methylbenzaldehyde (3 g, 15 mmol) in methanol (30 mL). The reaction mixture was stirred overnight at room temperature and the solvent was evaporated under reduced pressure. The residue was partitioned between water/ethyl acetate (50 mL/50 mL). The organic layer was separated, dried over sodium sulfate, filtered and evaporated under reduced pressure to afford (2-bromo-4-methylphenyl)methanol (2.8 g, 13.9 mmol, 90% purity, 83.7% yield) that was used for the next step without further purification.

### Step 2. Synthesis of 2-bromo-1-(chloromethyl)-4-methylbenzene

Thionyl chloride (1.97 g, 16.6 mmol) was added dropwise to a solution of (2-bromo-4-methylphenyl)methanol (2.8 g, 13.9 mmol) in dichloromethane (30 mL) and the mixture was stirred overnight at room temperature. After reaction completion (NMR control) the mixture was washed with water (30 mL×3) and concentrated in vacuum to afford 2-bromo-1-(chloromethyl)-4-methylbenzene (2.3 g, 10.4 mmol, 85% purity, 63.9% yield) that was used in next step without further purification.

### Step 3. Synthesis of 2-(2-bromo-4-methylphenyl)acetonitrile

Potassium cyanide (1.01 g, 15.6 mmol) was added to a solution of 2-bromo-1-(chloromethyl)-4-methylbenzene (2.3 g, 10.4 mmol) in DMSO (20 mL), the mixture was stirred overnight and diluted with water (40 mL). The product was extracted with ethyl acetate (50 mL×2), combined organic layers were dried over sodium sulfate, filtered and evaporated under reduced pressure to afford 2-(2-bromo-4-methylphenyl)acetonitrile (1.7 g, 8.09 mmol, 90% purity, 70.1% yield) that was used in next step without further purification.

### Step 4. Synthesis of 1-(2-bromo-4-methylphenyl)cyclopentane-1-carbonitrile

2-(2-bromo-4-methylphenyl)acetonitrile (1.7 g, 8.09 mmol) was added portionwise to the suspension of sodium hydride (0.966 g, 24.2 mmol, 60 w %) in DMF (20 mL) at 0° C. The mixture was stirred 30 min at this temperature and 1,4-dibromobutane (1.74 g, 8.09 mmol) was added portionwise at the same temperature. After the mixture was allowed to warm up to room temperature and stir overnight until completion. The solvent was evaporated under reduced pressure, the residue was treated with water/ethyl acetate mixture (30 mL/30 mL). The organic layer was separated, dried over sodium sulfate, filtered and evaporated under reduced pressure. The crude product was purified by flash chromatography (hexane/MTBE) to afford 1-(2-bromo-4-methylphenyl)cyclopentane-1-carbonitrile (0.6 g, 2.27 mmol, 95% purity, 26.7% yield).

### Step 5. Synthesis of 6′-methyl-1′,2′-dihydrospiro[cyclopentane-1,3′-indol]-2′-one

Potassium iodide (0.038 g, 0.228 mmol), copper iodide (0.043 g, 0.225 mmol), and N-acetylglycine (0.027 g, 0.230 mmol) were added to a solution of 1-(2-bromo-4-methylphenyl)cyclopentane-1-carbonitrile (0.6 g, 2.27 mmol) and sodium hydroxide (0.272 g, 6.81 mmol) in tert-butanol (20 mL). The mixture was refluxed for 24 h, then cooled to room temperature, filtered through silica, and the filtrate was evaporated to dryness. The residue was treated with water/ethyl acetate mixture (50 mL/50 mL). The organic layer was separated, dried over sodium sulfate, filtered and evaporated under reduced pressure. The crude product was purified by flash chromatography (chloroform/MTBE) to afford 6′-methyl-1′,2′-dihydrospiro[cyclopentane-1,3′-indol]-2′-one (0.3 g, 1.49 mmol, 90% purity, 59.2% yield) that was used in next step without further purification.

### Step 6. Synthesis of 6′-methyl-1′,2′-dihydrospiro[cyclopentane-1,3′-indole]

10 M dimethylsulfide borane complex solution in THF (0.45 mL, 0.339 g, 4.47 mmol) was added to a solution of 6′-methyl-1′,2′-dihydrospiro[cyclopentane-1,3′-indol]-2′-one (0.3 g, 1.49 mmol) in dry THF (20 mL), the mixture was refluxed for 2 h and cooled to room temperature. Then methanol (10 mL) was added dropwise, the mixture was refluxed for 2 h, cooled to room temperature and evaporated under reduced pressure. The residue was treated with water/ethyl acetate mixture (20 mL/20 mL). The organic layer was separated, dried over sodium sulfate, filtered and evaporated under reduced pressure to afford crude 6′-methyl-1′,2′-dihydrospiro[cyclopentane-1,3′-indole] (0.25 g, 1.33 mmol, 75% purity, 67% yield) that was used in the next step without further purification.

### Step 7. Synthesis of N,N-dimethyl-4-({6′-methyl-1′,2′-dihydrospiro[cyclopentane-1,3′-indol]-1′-yl}sulfonyl)benzene-1-sulfonamide

4-(dimethylsulfamoyl)benzene-1-sulfonyl chloride (0.377 g, 1.33 mmol) was added to the mixture of 6′-methylspiro[cyclopentane-1,3′-indoline] (0.25 g, 1.33 mmol) and pyridine (0.157 g, 1.99 mmol) in dry tetrahydrofuran (20 mL). The reaction mixture was stirred overnight and evaporated to dryness. The residue was subjected to HPLC purification (deionized water/HPLC-grade methanol) that afforded the product as pink solid (0.0596 g, 0.137 mmol, 95% purity, 9.8% yield). The analytical data provided for this compound provisionally supports the proposed structure for N,N-dimethyl-4-((6′-methylspiro[cyclopentane-1,3′-indolin]-1′-yl)sulfonyl)benzenesulfonamide, I-79. Yield: 59.6 mg, 9.8%; Appearance Pink solid; ¹H NMR (400 MHz, DMSO-d₆) δ 8.04 (d, J=8.5 Hz, 2H), 7.97-7.88 (m, 2H), 7.34 (s, 1H), 7.05 (d, J=7.7 Hz, 1H), 6.89 (d, J=7.8 Hz, 1H), 3.73 (s, 2H), 2.33 (s, 3H), 1.73-1.54 (m, 4H), 1.48 (dd, J=12.8, 6.1 Hz, 2H), 1.31 (dd, J=11.8, 6.5 Hz, 2H). HPLC purity: 100%; LCMS Calculated for C₂₁H₂₆N₂O₄S₂: 434.57; Observed 435.2 [M−H]⁺.

### Example 17—Synthesis of benzyl 1-(benzylsulfonyl)spiro[indoline-3,4′-piperidine]-1′-carboxylate (I-80)

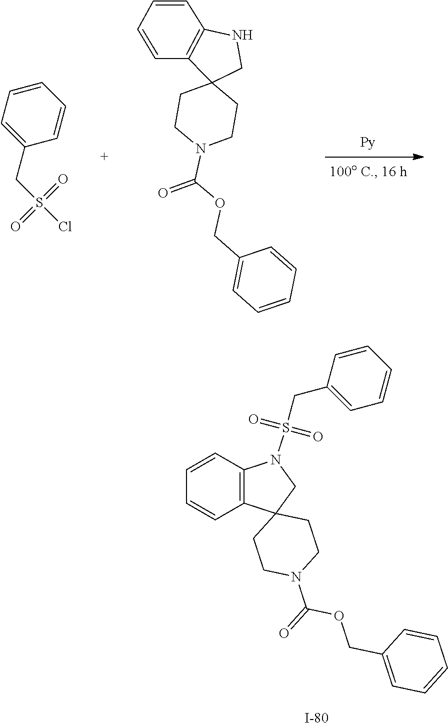

Phenylmethanesulfonyl chloride (0.066 g, 0.346 mmol) was added to the vial containing benzyl 1,2-dihydrospiro[indole-3,4′-piperidine]-1′-carboxylate (0.101 g, 0.313 mmol) in dry pyridine (1 mL). The reaction mixture was heated at 100° C. with stirring for 16 h. After cooling to the room temperature the mixture was evaporated. The residue was dissolved in DMSO (2 mL), filtered from non-soluble impurities. The resulting filtrate was subjected to HPLC purification (deionized water/HPLC-grade acetonitrile) that afforded benzyl 1-(benzylsulfonyl)spiro[indoline-3,4′-piperidine]-1′-carboxylate, I-80. Yield: 28.8 mg, 16.6%; Appearance: Pink solid; ¹H NMR (400 MHz, DMSO-d₆) δ 7.42-7.23 (m, 10H), 7.13 (dd, 1=15.1, 7.6 Hz, 3H), 7.03-6.88 (m, 1H), 5.06 (s, 2H), 4.50 (s, 2H), 4.02 (d, J=13.7 Hz, 2H), 3.68 (s, 2H), 1.71 (td, J=13.0, 4.5 Hz, 2H), 1.54 (d, J=13.5 Hz, 2H); HPLC purity: 100%; LCMS Calculated for C₂₇H₂₈N₂O₄S₂: 476.59; Observed: 475.0 [M−H]⁻.

### Example 18

The following example compound was prepared using standard chemical manipulations and procedures similar to those used in Example 17.

| Compound | | |
|---|---|---|
| No. | Structure | Analytical data |
| I-81 | 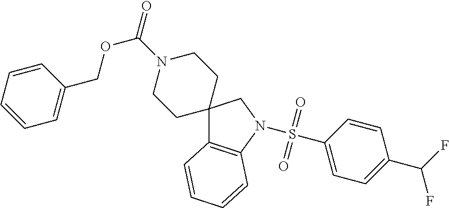 [SMILES: O=C(OCc1ccccc1)N1CCC2(CC1)CN(S(=O)(=O)c1ccc(C(F)F)cc1)c1ccccc12] | Yield: 54.3 mg, 31.2%; Appearance: Pink solid; ¹H NMR (600 MHz, DMSO-d₆) δ 7.96 (d, J = 8.1 Hz, 2H), 7.74 (d, J = 8.0 Hz, 2H), 7.47 (d, J = 8.1 Hz, 1H), 7.37-7.29 (m, 4H), 7.23-6.97 (m, 4H), 5.05 (d, J = 7.7 Hz, 2H), 3.90 (s, 2H), 3.86 (d, J = 13.7 Hz, 2H), 2.92 (d, J = 50.7 Hz, 2H), 1.55 (td, J = 13.1, 4.5 Hz, 2H), 1.07 (d, J = 13.2 Hz, 2H); HPLC purity: 100%; LCMS Calculated for C₂₇H₂₆F₂N₂O₄S: 512.57; Observed: 513.2 [M + H]⁺. |

### Example 19—Synthesis of 1′-methyl-1-phenylmethanesulfonyl-1,2-dihydrospiro[indole-3,4′-piperidine] (I-82)

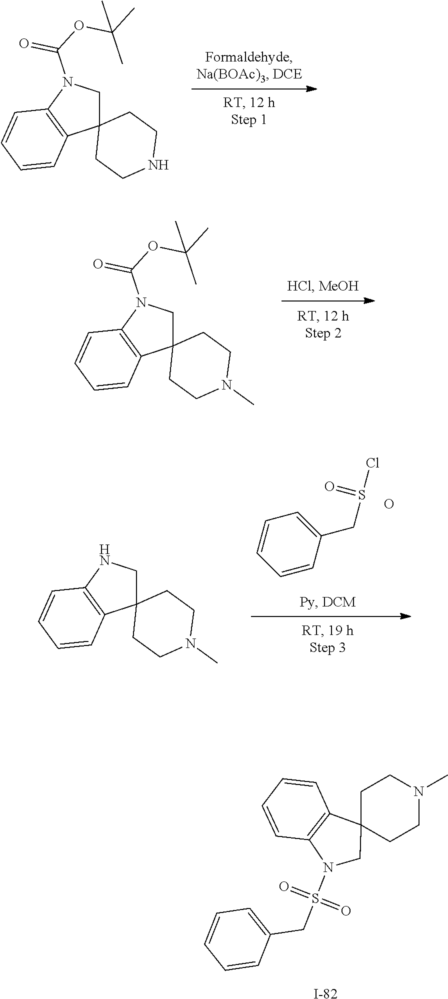

### Step 1. Synthesis of tert-butyl 1′-methyl-1,2-dihydrospiro[indole-3,4′-piperidine]-1-carboxylate

A mixture of tert-butyl 1,2-dihydrospiro[indole-3,4′-piperidine]-1-carboxylate (5 g, 17.3 mmol) and formaldehyde 37% aq. solution (5.83 mL, 78.9 mmol) in 1,2-dichloroethane (50 mL) was stirred at room temperature for 20 min followed by addition of sodium triacetoxyborohydride (8.14 g, 38.5 mmol). The reaction mixture was stirred for 15 h at room temperature and diluted with 1M sodium hydroxide aq. solution (100 mL). The product was extracted with dichloromethane (100 mL×2). The combined organic layers were dried over sodium sulfate, filtered and evaporated to dryness to afford tert-butyl 1′-methyl-1,2-dihydrospiro[indole-3,4′-piperidine]-1-carboxylate (6 g, 19.8 mmol, 80% purity, 91.7% yield) that was used in next step without further purification.

### Step 2. Synthesis of 1′-methyl-1,2-dihydrospiro[indole-3,4′-piperidine] dihydrochloride

tert-butyl 1′-methyl-1,2-dihydrospiro[indole-3,4′-piperidine]-1-carboxylate (4 g, 13.2 mmol) was dissolved in 1M HCl solution in methanol (100 mL). The reaction mixture was stirred for 1 h at room temperature, evaporated to dryness. Obtained residue was treated with ether (100 mL), formed solid was filtered-off, washed with ether (100 mL) and dried on air to give 1′-methyl-1,2-dihydrospiro[indole-3,4′-piperidine] dihydrochloride as yellow solid (3.5 g, 12.7 mmol, 95% purity, 91.4% yield).

### Step 3. Synthesis of 1′-methyl-1-phenylmethanesulfonyl-1,2-dihydrospiro[indole-3,4′-piperidine]

Phenylmethanesulfonyl chloride (0.434 g, 2.28 mmol) was added to a solution of 1′-methyl-1,2-dihydrospiro[indole-3,4′-piperidine] dihydrochloride (0.6 g, 2.18 mmol) and pyridine (0.517 g, 6.54 mmol) in dichloromethane (10 mL), the reaction mixture was stirred for 16 h at room temperature and diluted with water (10 mL). The organic layer was separated, dried over sodium sulfate, filtered and evaporated under reduced pressure. The residue was purified by HPLC purification (deionized water/HPLC-grade methanol, ammonia) to give 1′-methyl-1-phenylmethanesulfonyl-1,2-dihydrospiro[indole-3,4′-piperidine], I-82. Yield: 135.0 mg, 16.4%; Appearance: Beige solid; ¹H NMR (600 MHz, DMSO-d₆) δ 7.29 (dq, J=7.3, 4.8, 3.7 Hz, 5H), 7.21 (d, J=7.5 Hz, 1H), 7.15-7.06 (m, 2H), 6.96 (ddd, J=7.9, 6.2, 2.2 Hz, 1H), 4.61 (s, 2H), 3.61 (s, 2H), 2.67 (d, J=10.8 Hz, 2H), 2.14 (s, 3H), 1.87-1.69 (m, 4H), 1.53-1.39 (m, 2H). HPLC purity: 100%; LCMS Calculated for CH₂₄N₂O₂S: 356.48; Observed: 357.4 [M−H]⁺.

### Example 20

The following compounds were prepared using standard chemical manipulations and procedures similar to those used for the preparation in Example 19.

| Compound | | |
|---|---|---|
| No. | Structure | Analytical data |
| I-83 | 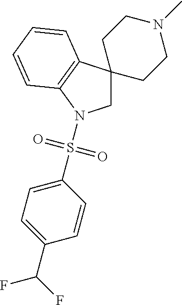 [SMILES: CN1CCC2(CC1)CN(S(=O)(=O)c1ccc(C(F)F)cc1)c1ccccc12] | Yield: 305.7 mg, 33.9%; Appearance: Beige solid; ¹H NMR (600 MHz, DMSO-d₆) δ 7.95 (d, J = 8.1 Hz, 2H), 7.74 (d, J = 8.0 Hz, 2H), 7.47 (d, J = 8.0 Hz, 1H), 7.23- 6.97 (m, 4H), 3.78 (s, 2H), 2.58 (dt, J = 12.3, 3.2 Hz, 2H), 2.14 (s, 3H), 1.92-1.83 (m, 2H), 1.64 (td, J = 13.0, 4.0 Hz, 2H), 1.05-0.95 (m, 2H); HPLC purity: 100%; LCMS Calculated for C₂₀H₂₂F₂N₂O₂S: 392.46; Observed: 393.2 [M + H]⁺. |
| I-84 | 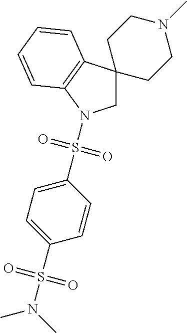 | Yield: 250.9 mg, 24.2%; Appearance: Beige solid; ¹H NMR (400 MHz, DMSO-d₆) δ 8.05 (dd, J = 8.5, 1.7 Hz, 2H), 7.94-7.87 (m, 2H), 7.53 (d, J = 8.1 Hz, 1H), 7.31- 7.23 (m, 1H), 7.22-7.17 (m, 1H), 7.08 (td, J = 7.5, 1.2 Hz, 1H), 3.82 (s, 2H), 2.58 (d, J = 1.4 Hz, 8H), 2.16 (d, J = 1.4 Hz, 3H), 1.89 (t, J = 12.0 Hz, 2H), 1.72-1.62 (m, 2H), 0.97 (d, J = 12.8 Hz, 2H); HPLC purity: 100%; LCMS Calculated for C₂₁H₂₇N₃O₄S₂: 449.59; Observed: 450.2 [M + H]⁺. |

### Example 21—Synthesis of 1-phenylmethanesulfonyl-1′-(2,2,2-trifluoroethyl)-1,2-dihydrospiro[indole-3,4′-piperidine] (I-85)

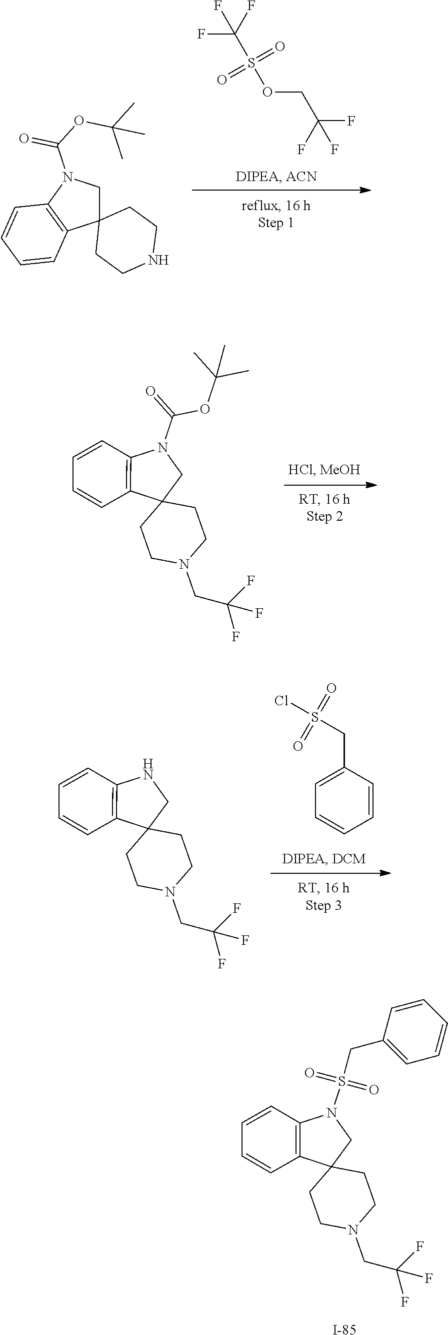

### Step 1. Synthesis of tert-butyl 1′-(2,2,2-trifluoroethyl)-1,2-dihydrospiro[indole-3,4′-piperidine]-1-carboxylate

2,2,2-trifluoroethyl trifluoromethanesulfonate (1.71 g, 7.37 mmol) was added to the mixture of tert-butyl spiro[indoline-3,4′-piperidine]-1-carboxylate hydrochloride (2 g, 6.15 mmol) and ethylbis(propan-2-yl)amine (2.37 g, 18.4 mmol) in dry acetonitrile (25 mL). The reaction mixture was refluxed overnight, cooled to room temperature, poured into NaHCO₃ sat. aq. solution (15 mL) and extracted with ethyl acetate (50 mL×2). Combined organic layers were dried over anhydrous sodium sulfate, filtered and evaporated under reduced pressure to afford tert-butyl 1′-(2,2,2-trifluoroethyl)-1,2-dihydrospiro[indole-3,4′-piperidine]-1-carboxylate (1.9 g, 5.12 mmol, 91% purity, 75.7% yield) that was used in the next step without further purification.

### Step 2. Synthesis of 1′-(2,2,2-trifluoroethyl)-1,2-dihydrospiro[indole-3,4′-piperidine]

tert-butyl 1′-(2,2,2-trifluoroethyl)spiro[indoline-3,4′-piperidine]-1-carboxylate (1.9 g, 5.12 mmol) was added to a stirred 1.5 M HCl solution in MeOH (20 mL), the resulting mixture was stirred overnight and evaporated. Crude residue was treated with NaHCO₃ sat aq. solution to reach pH=8 and the product was extracted with dichlorimethane (20 mL×3). Organic layers were combined, dried over sodium sulfate, filtered and evaporated under reduced pressure to give 1′-(2,2,2-trifluoroethyl)-1,2-dihydrospiro[indole-3,4′-piperidine] (1.1 g, 4.06 mmol, 95% purity, 75.3% yield).

### Step 3. Synthesis of 1-phenylmethanesulfonyl-1′-(2,2,2-trifluoroethyl)-1,2-dihydrospiro[indole-3,4′-piperidine]

Phenylmethanesulfonyl chloride (0.192 g, 1.01 mmol) was added to the mixture of 1′-(2,2,2-trifluoroethyl)spiro[indoline-3,4′-piperidine] (0.25 g, 0.924 mmol) and ethylbis(propan-2-yl)amine (0.178 g, 1.38 mmol) in dry dichloromethane (5 mL). The reaction mixture was stirred overnight, poured into NaHCO₃ aq. solution (15 mL) and extracted with dichloromethane (20 mL). Combined organic layers were dried over anhydrous sodium sulfate, filtered and evaporated under reduced pressure. Resulting solid was purified by HPLC (deionized water/HPLC-grade acetonitrile) to afford 1-phenylmethanesulfonyl-1′-(2,2,2-trifluoroethyl)-1,2-dihydrospiro[indole-3,4′-piperidine], I-85. Yield: 137.2 mg, 33.1%; Appearance: White solid; ¹H NMR (400 MHz, DMSO-d₆) δ 7.37-7.19 (m, 6H), 7.14-7.03 (m, 2H), 6.95 (ddt, J=7.7, 5.6, 2.6 Hz, 1H), 4.60 (d, J=2.3 Hz, 2H), 3.63 (s, 2H), 3.14 (q, J=10.4 Hz, 2H), 2.82 (d, J=11.7 Hz, 2H), 2.31 (t, J=12.1 Hz, 2H), 1.78 (td, J=12.9, 4.0 Hz, 2H), 1.46 (d, J=13.1 Hz, 2H). HPLC purity: 100%; LCMS Calculated for C₂₁H₂₃F₃N₂O₂S: 424.48; Observed: 425.2 [M+H]⁺.

### Example 22

The following compounds were prepared using standard chemical manipulations and procedures similar to those used for the preparation of Example 21.

| Compound | | |
|---|---|---|
| No. | Structure | Analytical data |
| I-86 | 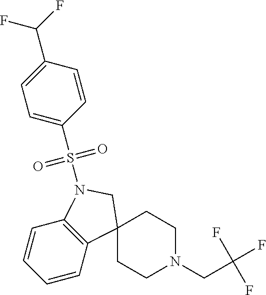 [SMILES: O=S(=O)(c1ccc(C(F)F)cc1)N1CC2(CCN(CC(F)(F)F)CC2)c2ccccc21] | Yield: 80.8 mg, 18.0%; Appearance: White solid; ¹H NMR (400 MHz, DMSO-d₆) δ 7.94 (d, J = 8.1 Hz, 2H), 7.72 (d, J = 8.0 Hz, 2H), 7.48-7.41 (m, 1H), 7.21 (dq, J = 7.3, 4.6, 3.2 Hz, 2H), 7.13-6.87 (m, 2H), 3.80 (d, J = 2.5 Hz, 2H), 3.13 (q, J = 10.7, 10.3 Hz, 2H), 2.74 (d, J = 11.8 Hz, 2H), 2.37 (t, J = 12.1 Hz, 2H), 1.66 (t, J = 12.8 Hz, 2H), 1.04 (d, J = 13.1 Hz, 2H). HPLC purity: 100%; LCMS Calculated for C₂₁H₂₁F₅N₂O₂S: 460.46; Observed: 461.2 [M + H]⁺. |
| I-87 | 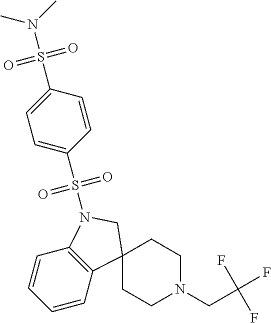 [SMILES: CN(C)S(=O)(=O)c1ccc(S(=O)(=O)N2CC3(CCN(CC(F)(F)F)CC3)c3ccccc32)cc1] | Yield: 77.2 mg, 15.3%; Appearance: White solid; ¹H NMR (500 MHz, DMSO-d₆) δ 8.04 (d, J = 8.2 Hz, 2H), 7.90 (d. J = 8.2 Hz, 2H), 7.51 (d, J = 8.2 Hz, 1H), 7.25 (d, J = 7.5 Hz, 2H), 7.06 (t, J = 7.5 Hz, 1H), 3.84 (s, 2H), 3.15 (q, J = 10.2 Hz, 2H), 2.74 (d, J = 11.9 Hz, 2H), 2.61 (s, 6H), 2.37 (t, J = 12.2 Hz, 2H), 1.67 (td, J = 13.0, 4.1 Hz, 2H), 1.00 (d, J = 13.0 Hz, 2H). HPLC purity: 100%; LCMS Calculated for C₂₂H₂₆F₃N₃O₄S₂: 517.58; Observed: 518.2 [M + H]⁺. |

### Example 23—Synthesis of 1′-methyl-1-phenylmethanesulfonyl-1,2-dihydrospiro[indole-3,3′-pyrrolidine] (I-88)

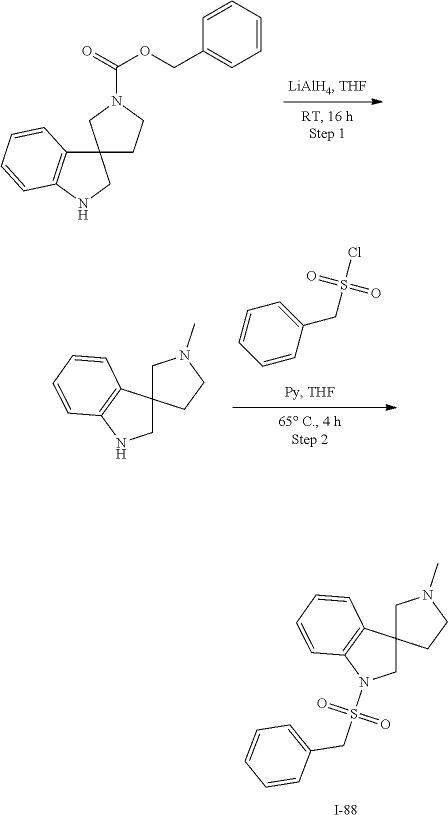

### Step 1. Synthesis of 1′-methyl-1,2-dihydrospiro[indole-3,3′-pyrrolidine]

Lithium aluminum hydride (0.75 g, 19.7 mmol) was suspended in anhydrous THF (20 mL). The solution of benzyl 1,2-dihydrospiro[indole-3,3′-pyrrolidine]-1′-carboxylate (1.3 g, 4.21 mmol) in anhydrous THF (5 mL) was added dropwise to this suspension, and the reaction mixture was stirred at room temperature overnight. The reaction was quenched with 20% aqueous solution of sodium hydroxide (25 mL). Solids were removed by filtration and washed with ethyl acetate (20 mL×2). The combined organic phase was washed with brine (20 mL), dried over sodium sulfate, filtered and evaporated under reduced pressure. The crude product was purified by HPLC (deionized water/HPLC-grade methanol, ammonia) to give 1′-methyl-1,2-dihydrospiro[indole-3,3′-pyrrolidine] (0.34 g, 1.8 mmol, 95% purity, 40.7% yield).

### Step 2. Synthesis of for 1′-methyl-1-phenylmethanesulfonyl-1,2-dihydrospiro[indole-3,3′-pyrrolidine]

phenylmethanesulfonyl chloride (0.125 g, 0.655 mmol) was added to the mixture of 1′-methyl-1,2-dihydrospiro[indole-3,3′-pyrrolidine] (0.12 g, 0.637 mmol) and pyridine (0.1 g, 1.27 mmol) in dry THF (3 mL). The reaction mixture was refluxed for 4 h, cooled to room temperature and evaporated to dryness. The crude residue was purified by HPLC (deionized water/HPLC-grade acetonitrile, ammonia) to give 1′-methyl-1-phenylmethanesulfonyl-1,2-dihydrospiro[indole-3,3′-pyrrolidine], I-88. Yield: 56.8 mg, 24.78%; Appearance: Yellow oil; ¹H NMR (400 MHz, DMSO-d₆) δ 7.35-7.27 (m, 6H), 7.14 (d, J=9.5 Hz, 2H), 7.02 (t, J=7.1 Hz, 1H), 4.61 (s, 2H), 3.83-3.63 (m, 2H), 2.70 (s, 1H), 2.58 (d, J=9.1 Hz, 2H), 2.38 (d, J=9.0 Hz, 1H), 2.26 (s, 3H), 2.08-1.87 (m, 2H); HPLC purity: 100/o; LCMS Calculated for C₁₉H₂₂N₂O₂S: 342.46; Observed: 343.2 [M+H]⁺.

### Example 24

The following compounds were prepared using standard chemical manipulations and procedures similar to those used for the preparation of Example 23.

| Compound | | |
|---|---|---|
| No. | Structure | Analytical data |
| I-89 | 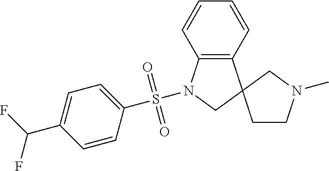 [SMILES: CN1CCC2(C1)CN(S(=O)(=O)c1ccc(C(F)F)cc1)c1ccccc12] | Yield: 141 mg, 55.1%; Appearance: Pink oil; ¹H NMR (400 MHz, DMSO-d₆) δ 7.96 (d, J = 8.0 Hz, 2H), 7.78 (d, J = 8.1 Hz, 2H), 7.48 (d, J = 8.1 Hz, 1H), 7.25 (t, J = 6.8 Hz, 2H), 7.14-6.91 (m, 2H), 3.90 (d, J = 10.8 Hz, 1H), 3.79 (d, J = 11.0 Hz, 1H), 2.65 (d, J = 7.5 Hz, 1H), 2.45 (d, J = 7.0 Hz, 1H), 2.24 (d, J = 9.4 Hz, 1H), 2.18 (d, J = 1.7 Hz, 3H), 2.15-2.06 (m, 1H), 1.92-1.80 (m, 1H), 1.79 (t, J = 7.1 Hz, 1H); HPLC purity: 100%; LCMS Calculated for C₁₉H₂₀F₂N₂O₂S: 378.44; Observed: 379.4 [M + H]⁺. |
| I-90 | 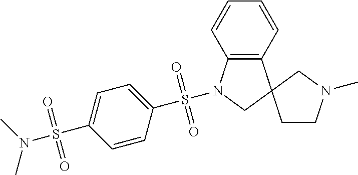 | Yield: 37.2 mg, 12.5%; Appearance: Beige solid; ¹H NMR (600 MHz, DMSO-d₆) δ 8.00 (d, J = 8.5 Hz, 2H), 7.90 (d, J = 8.3 Hz, 2H), 7.48 (d, J = 8.0 Hz, 1H), 7.25 (t, J = 7.9 Hz, 2H), 7.07 (t, J = 7.5 Hz, 1H), 3.89 (d, J = 11.1 Hz, 1H), 3.80 (d, J = 11.1 Hz, 1H), 2.57 (s, 7H), 2.42 (dt, J = 8.9, 4.4 Hz, 1H), 2.21- 2.10 (m, 5H), 1.78 (ddd, J = 14.1, 8.8, 5.6 Hz, 1H), 1.66 (ddd, J = 13.5, 8.2, 5.9 Hz, 1H); HPLC purity: 100%; LCMS Calculated for C₂₀H₂₅N₃O₄S₂: 435.56; Observed: 436.2 [M + H]⁺. |

### Example 25—Synthesis of benzyl 1-[4-(difluoromethyl)benzenesulfonyl]-1,2-dihydrospiro[indole-3,3′-pyrrolidine]-1′-carboxylate (I-91)

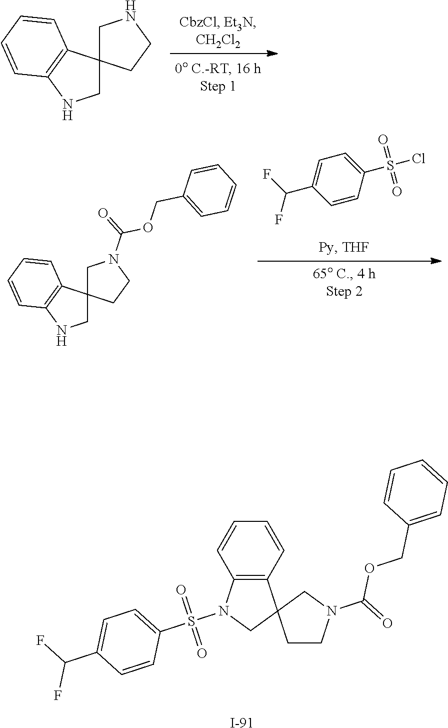

### Step 1. Synthesis of benzyl 1,2-dihydrospiro[indole-3,3′-pyrrolidine]-1′-carboxylate

1,2-dihydrospiro[indole-3,3′-pyrrolidine] dihydrochloride (1.5 g, 6.06 mmol) and triethylamine (2.14 g, 21.2 mmol) were dissolved in dichloromethane (50 mL), the mixture was stirred for 30 min, cooled to 0° C. and benzyl carbonochloridate (1.03 g, 6.06 mmol) was added dropwise to it. The mixture was warmed to room temperature and stirred overnight, then washed with water (50 mL) and brine (50 mL). The organic layer was dried over sodium sulfate, filtered and evaporated under reduced pressure to afford benzyl 1,2-dihydrospiro[indole-3,3′-pyrrolidine]-1′-carboxylate (1.8 g, 5.83 mmol, 85% purity, 82.2% yield) that was used in next step without further purification.

### Step 2. Synthesis of benzyl 1-[4-(difluoromethyl)benzenesulfonyl]-1,2-dihydrospiro[indole-3,3′-pyrrolidine]-1′-carboxylate

4-(difluoromethyl)benzene-1-sulfonyl chloride (0.22 g, 0.972 mmol) was added to the mixture of benzyl 1,2-dihydrospiro[indole-3,3′-pyrrolidine]-1′-carboxylate (0.3 g, 0.972 mmol) and pyridine (0.230 g, 2.91 mmol) in dry THF (10 mL). The reaction mixture was refluxed for 4 h, cooled to room temperature and evaporated to dryness. The crude residue was purified by HPLC (deionized water/HPLC-grade acetonitrile) to give benzyl 1-[4-(difluoromethyl)benzenesulfonyl]-1,2-dihydrospiro[indole-3,3′-pyrrolidine]-1′-carboxylate, 1-91. Yield: 88.5 mg, 17.3%; Appearance: Pink oil; ¹H NMR (400 MHz, DMSO-d₆) δ 7.98 (d, J=7.8 Hz, 2H), 7.75 (s, 2H), 7.52 (d, J=8.2 Hz, 1H), 7.45-7.28 (m, 5H), 7.23 (d, J=7.4 Hz, 1H), 7.15-6.98 (m, 2H), 5.08 (d, J=9.5 Hz, 2H), 3.93 (s, 2H), 3.30-3.24 (m, 2H), 1.93 (d, J=11.7 Hz, 1H), 1.77 (s, 1H)); HPLC purity: 100%; LCMS Calculated for C₂₉H₂₅N₃O₄S₂: 498.54; Observed: 499.2[M+H]⁺.

### Example 26—Synthesis of 1-[4-(difluoromethyl)benzenesulfonyl]-1′-(2,2,2-trifluoroethyl)-1,2-dihydrospiro[indole-3,3′-pyrrolidine] (I-92)

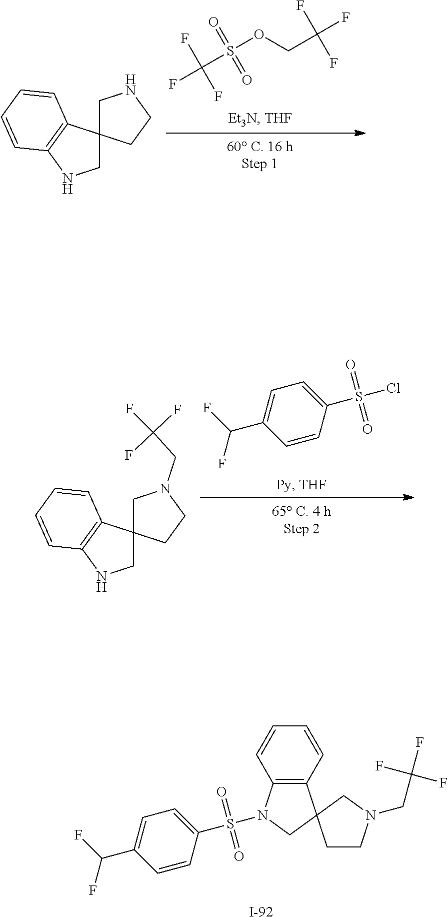

### Step 1. Synthesis of 1′-(2,2,2-trifluoroethyl)-1,2-dihydrospiro[indole-3,3′-pyrrolidine]

2,2,2-trifluoroethyl trifluoromethanesulfonate (1.87 g, 8.09 mmol) was added dropwise to a mixture of 1,2-dihydrospiro[indole-3,3′-pyrrolidine] dihydrochloride (2 g, 8.09 mmol) and triethylamine (3.26 g, 32.3 mmol) in THF (30 mL). The mixture was stirred at 60° C. overnight, cooled to room temperature and concentrated under the reduced pressure. The residue was dissolved in ethyl acetate (50 mL), the solution was washed with water (50 mL), brine (50 mL), dried over sodium sulfate, filtered and concentrated under reduced pressure to give 1′-(2,2,2-trifluoroethyl)-1,2-dihydrospiro[indole-3,3′-pyrrolidine] (1.65 g, 6.43 mmol, 87% purity, 69% yield) that was used in next step without further purification.

### Step 2. Synthesis of 1-[4-(difluoromethyl)benzenesulfonyl]-1′-(2,2,2-trifluoroethyl)-1,2-dihydrospiro-[indole-3,3′-pyrrolidine]

4-(difluoromethyl)benzene-1-sulfonyl chloride (0.2 g, 0.882 mmol) was added to the mixture of 1′-(2,2,2-trifluoroethyl)-1,2-dihydrospiro[indole-3,3′-pyrrolidine] (0.22 g, 0.858 mmol) and pyridine (0.203 g, 2.57 mmol) in dry THF (3 mL). The reaction mixture was refluxed for 4 h, cooled to room temperature and evaporated to dryness. The crude residue was purified by HPLC (deionized water/HPLC-grade acetonitrile) to give 1-[4-(difluoromethyl)benzenesulfonyl]-1′-(2,2,2-trifluoroethyl)-1,2-dihydrospiro[indole-3,3′-pyrrolidine], 1-92. Yield: 172 mg, 42.5%; Appearance: Pink solid; ¹H NMR (600 MHz, DMSO-d₆) δ 7.93 (d, J=8.1 Hz, 2H), 7.74 (d, J=8.1 Hz, 2H), 7.47 (d, J=8.0 Hz, 1H), 7.27-6.95 (m, 4H), 3.93-3.76 (m, 2H), 3.23 (qq, J=10.5, 5.0 Hz, 2H), 2.88 (td, J=8.5, 6.4 Hz, 1H), 2.78 (td, J=9.0, 5.3 Hz, 1H), 2.49 (s, 2H), 1.85 (ddd, J=13.1, 8.8, 6.4 Hz, 1H), 1.74 (ddd, J=13.2, 8.2, 5.4 Hz, 1H). HPLC purity: 100%; LCMS Calculated for C₂₀H₁₉F₅N₂O₂S: 446.43; Observed: 447.2 [M+H]⁺.

### Example 27

The following compound was prepared using standard chemical manipulations and procedures similar to those used for the preparation of Example 26.

| Compound | | |
|---|---|---|
| No. | Structure | Analytical data |
| I-93 | 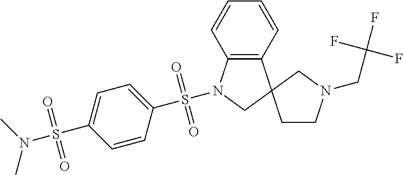 [SMILES: CN(C)S(=O)(=O)c1ccc(S(=O)(=O)N2CC3(CCN(CC(F)(F)F)C3)c3ccccc32)cc1] | Yield: 71.7 mg, 15.7%; Appearance: White solid; ¹H NMR (600 MHz, DMSO-d₆) δ 8.02 (d, J = 8.4 Hz, 2H), 7.89 (d, J = 8.3 Hz, 2H), 7.49 (d, J = 8.0 Hz, 1H), 7.26 (t, J = 7.8 Hz, 2H), 7.08 (t, J = 7.5 Hz, 1H), 3.91-3.80 (m, 2H), 3.23 (ddt, J = 14.8, 10.8, 4.5 Hz, 2H), 2.91-2.83 (m, 1H), 2.78 (td, J = 9.0, 5.3 Hz, 1H), 2.57 (s, 6H), 2.57-2.52 (m, 2H), 1.77 (ddd, J = 13.0, 8.8, 6.4 Hz, 1H), 1.66 (ddd, J = 13.2, 8.1, 5.3 Hz, 1H); HPLC purity: 100%; LCMS Calculated for C₂₁H₂₄F₃N₃O₄S₂: 503.56; Observed: 504.2 [M + H]⁺. |

### Example 28—Synthesis of 1′-[(3,3-dimethyl-2,3-dihydro-1-benzofuran-5-yl)sulfonyl]-1′,2′-dihydrospiro[cyclohexane-1,3′-indole] (I-94)

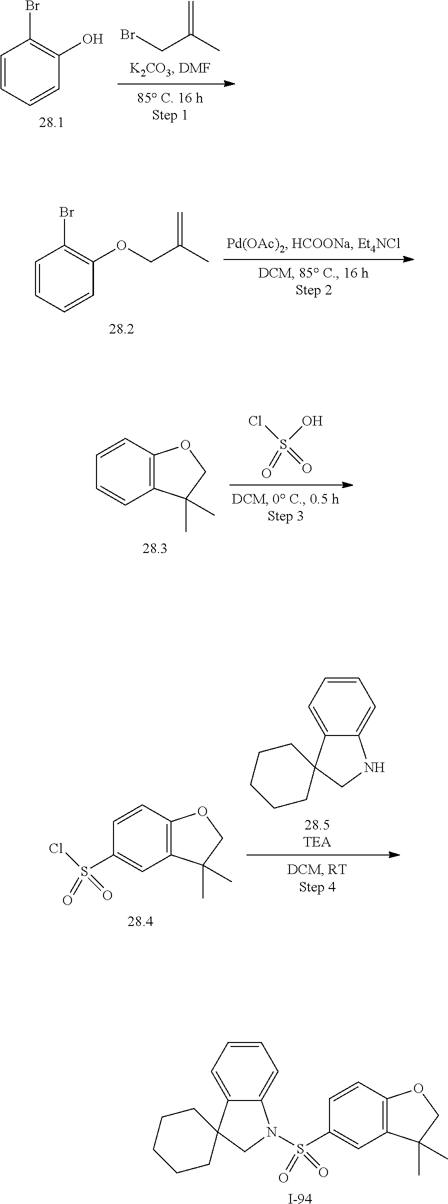

### Step-1: Procedure for Synthesis of 1-bromo-2-((2-methylallyl)oxy)benzene 28.2

To a stirred solution of 2-bromophenole 28.1 (5.0 g, 28.90 mmol, 1.0 eq.) in acetonitrile (50 mL) was added 3-bromo-2-methylprop (4.27 g, 31.70 mmol, 1.1 eq.) and potassium carbonate (9.97 g, 72.20 mmol, 2.5 eq.) at room temperature. The reaction mixture was heated to 85° C. and stirred for 16 h. The progress of the reaction was monitored by LCMS. The residue was filtered and concentrated under reduced pressure to get the crude. The crude product was purified by column chromatography (0-10%, EA in PE) on silica gel to afford 1-bromo-2-((2-methylallyl)oxy)benzene 28.2 (6.6 g, 100%) as colorless oil. LCMS: 227.1 [M+H]⁺.

### Step-2: Procedure for Synthesis of 3,3-dimethyl-2,3-dihydrobenzofuran 28.3

To a stirred solution of 1-bromo-2-((2-methylallyl)oxy)benzene 28.2 (3.0 g, 13.20 mmol, 1.0 eq) in N,N-Dimethylformamide (60 mL) was added Sodium Formate (1.07 g, 15.80 mmol, 1.2 eq.), sodium acetate (2.7 g, 33.0 mmol, 2.5 eq), Tetraethyl ammonium chloride (2.61 g, 15.80 mmol, 1.2 eq.) and palladium acetate (296 mg, 1.32 mmol, 0.1 eq.) at room temperature under N₂. The reaction mixture was heated to 85° C. and stirred for 16 h under N₂. The progress of the reaction was monitored by LCMS. After completion of reaction, the residue was added water and extracted with ethyl acetate. The organic layer was dried over anhydrous Na₂SO₄, filtered and concentrated under reduced pressure. The crude product was purified by column chromatography (PE) on silica gel to afford 3,3-dimethyl-2,3-dihydrobenzofuran 28.3 (1.34 g, 68.7%) as a colourless oil. LCMS: 149.1 [M+H]+.

### Step-3: Procedure for Synthesis of spiro[cyclopentane-1,3′-indoline 28.4

To the mixture of 3,3-dimethyl-2,3-dihydro-1-benzofuran 28.3 (188 mg, 1.26 mmol, 1.0 eq.) in DCM (8 mL) was added sulfurochloridic acid (220 mg, 1.89 mmol, 1.5 eq.) drop-wise at 0° C. The resulting mixture was stirred for 20 min at this temperature. When TLC show the reaction was completely. The resulting mixture was poured into ice-water (100 mL) and extracted with DCM (100 mL×3). The combined organic phase washed with brine, dried over Na₂SO₄ and concentrated in vacuum. The residue was purified by column chromatography (0-10/0 EA in PE) to give 3,3-dimethyl-2,3-dihydro-1-benzofuran-5-sulfonyl chloride 28.4 (69.3 mg, 281 μmol) as an off-white solid. LCMS: 243.3 [M-Cl+MeOH]⁺.

¹H NMR (400 MHz, DMSO-d₆) 7.39 (d, J=1.5 Hz, 1H), 7.36 (dd, J=8.2, 1.8 Hz, 1H), 6.66 (d, J=8.2 Hz, 1H), 1.28 (s, 6H).

### Step-4. Procedure for synthesis of 1′-[(3,3-dimethyl-2,3-dihydro-1-benzofuran-5-yl)sulfonyl]-1′,2′-dihydrospiro[cyclohexane-1,3′-indole]

To the mixture of 1′,2′-dihydrospiro[cyclohexane-1,3′-indole] (30 mg, 160 μmol, 1.0 eq), TEA (32.3 mg, 320 μmol, 2.0 eq.) in DCM (5 mL) was added 3,3-dimethyl-2,3-dihydro-1-benzofuran-5-sulfonyl chloride (39.4 mg, 160 μmol, 1.0 eq.) portion-wise at 20° C. The resulting mixture was stirred for 16 h at this temperature. Then the solution was poured into water and extracted with DCM (50 mL×3). The combined organic phase washed with brine, dried over Na₂SO₄ and concentrated in vacuum. The residue was purified by column chromatography (0-30% EA in PE) to give 1′-[(3,3-dimethyl-2,3-dihydro-1-benzofuran-5-yl)sulfonyl]-1′,2′-dihydrospiro[cyclohexane-1,3′-indole], I-94.

Yield: 26.5 mg, 41.1%; Appearance: Off-white solid; ¹H NMR (400 MHz, CDCl3) δ 7.65 (d, J=8.0 Hz, 1H), 7.57 (dd, J=8.4, 2.0 Hz, 1H), 7.49 (d, J=1.6 Hz, 1H), 7.17-7.22 (m, 1H), 6.99-7.01 (m, 2H), 6.73 (d, J=8.4 Hz, 1H), 4.27 (s, 2H) 3.75 (s, 2H), 1.59-1.70 (m, 3H) 1.35-1.40 (m, 2H), 1.17-1.32 (m, 11H); HPLC purity: 99.06%; LCMS calculated for C₂₃H₂₇NO₃S: 397.17; Observed: 398.4 [M+H]⁺.

### Example 29—Synthesis of 4-({4′-fluoro-1′,2′-dihydrospiro[cyclopentane-1,3′-indol]-1′-yl}sulfonyl)-N,N-dimethylbenzene-1-sulfonamide (I-95)

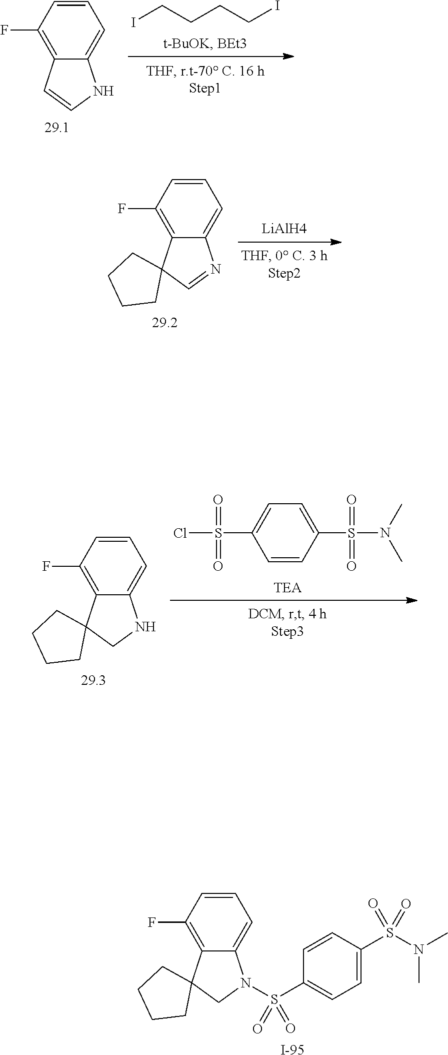

### Step-1: Procedure for Synthesis of 4′-fluorospiro[cyclopentane-1,3′-indole] 29.2

To a solution of 4-fluoro-1H-indole 29.1 (500 mg, 3.69 mmol, 1.0 eq.) in THF (10 mL) was added t-BuOK (910 mg, 8.11 mmol, 2.2 eq.), the reaction mixture was stirred at room temperature for 30 min under N₂. BEt₃ (7.38 mL, 7.38 mmol, 2.0 eq.) was added to the mixture, the mixture continue stirred 30 min, then 1,4-diiodobutane (1.25 g, 4.05 mmol, 1.1 eq.) was added to the mixture. The mixture was stirred at 70° C. for 16 h. The mixture was cooled to r.t, diluted with H₂O (20 mL), extracted with EA (30 mL×3). The organic layer was combined, dried over anhydrous Na₂SO₄, filtered and concentrated. The residue was purified with flash column chromatography (EA in PE, from 0/6 to 17%) to afford 4′-fluorospiro[cyclopentane-1,3′-indole]29.2 (300 mg, 43.0%) as a yellow solid. LCMS: 190.2 [M+H]⁺.

### Step-2: Procedure for Synthesis of 4′-fluorospiro[cyclopentane-1,3′-indoline] 29.3

To a solution of 4′-fluorospiro[cyclopentane-1,3′-indole] 29.2 (300 mg, 1.59 mmol, 1.0 eq.) in THF (5 mL) was slowly added LiAlH₄ (120.8 mg, 3.18 mmol, 2.0 eq.) at 0° C. The reaction mixture was stirred 0° C. for 3 h. The mixture was quenched with NH₄Cl (5 mL), extracted with EA (15 mL×3). The organic layer was combined, dried over anhydrous Na₂SO₄, filtered and concentrated. The residue was purified with flash column chromatography (EA in PE, from 0% to 20%) to afford 4′-fluorospiro[cyclopentane-1,3′-indoline] 29.3 (100 mg, 33%) as a yellow oil. LCMS: 192.2 [M+H]⁺.

### Step-3: Procedure for Synthesis of 4-((4′-fluorospiro[cyclopentane-1,3′-indolin]-1′-yl)sulfonyl)-N,N-dimethylbenzenesulfonamide

To a solution of 4′-fluoro-1′,2′-dihydrospiro[cyclopentane-1,3′-indole] (100 mg, 522 μmol, 1.0 eq.) in DCM (2 mL) were added 4-(dimethylsulfamoyl)benzene-1-sulfonyl chloride (148 mg, 522 μmol, 1.0 eq.) and TEA (157 mg, 1.56 mmol, 3.0 eq.). The reaction mixture was stirred at r.t for 4 h. Then the mixture was diluted with H₂O (10 mL), extracted with DCM (15 mL×3). The organic layer was combined, dried over anhydrous Na₂SO₄, filtered and concentrated. The residue was purified by prep-HPLC to afford 4-({4′-fluoro-1′,2′-dihydrospiro[cyclopentane-1,3′-indol]-1′-yl}sulfonyl)-N,N-dimethylbenzene-1-sulfonamide (I-95)

Yield: 45.9 mg, 20.0%; Appearance: White solid; ¹H NMR (400 MHz, CDCl3) δ 7.98-7.95 (m, 2H), 7.88-7.55 (m, 2H), 7.44 (dd, J=8.0 Hz, 0.8 Hz, 1H), 7.21-7.16 (m, 1H), 6.75-6.70 (m, 1H), 3.69 (s, 2H), 2.72 (s, 6H), 2.02-1.94 (m, 2H), 1.84-1.75 (m, 2H), 1.63-1.60 (m, 2H), 1.45-1.40 (m, 2H); HPLC purity: 95.43%; LCMS calculated for C₂₀H₂₃FN₂O₄S₂: 438.11; Observed: 439.1[M+H]⁺.

### Example 30—Synthesis of N,N-dimethyl-4-({1-[(oxan-4-yl)methyl]-1′,2′-dihydrospiro[azetidine-3,3′-indol-1′-yl}sulfonyl)benzene-1-sulfonamide (I-96)

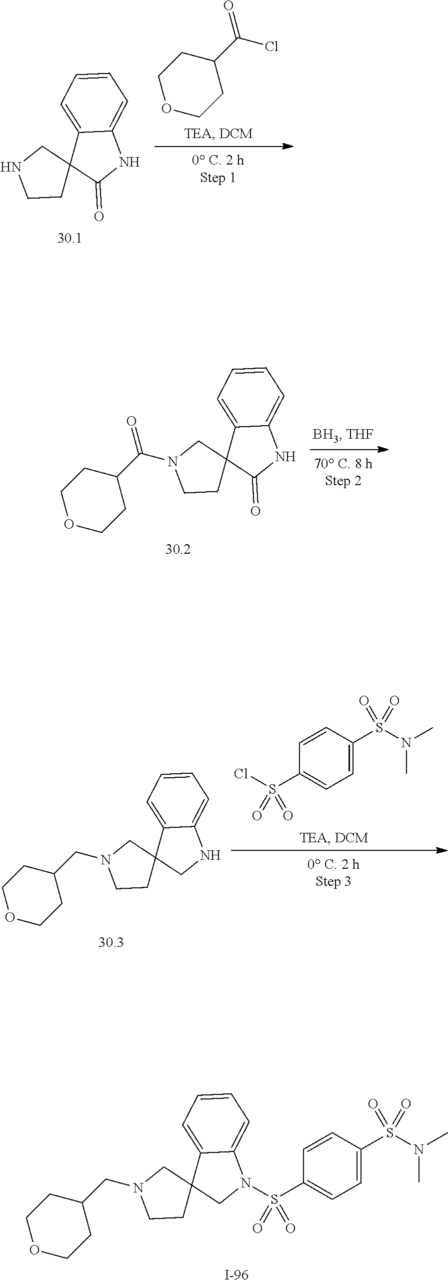

### Step-1: Procedure for Synthesis of 1′-(tetrahydro-2H-pyran-4-carbonyl)spiro[indoline-3,3′-pyrrolidin]-2-one 30.2

A 50 mL round-bottomed flask was charged with spiro[indoline-3,3′-pyrrolidin]-2-one 30.1 (100 mg, 531 μmol, 1 eq.), oxane-4-carbonyl chloride (86.7 mg, 584 μmol, 1.1 eq.), triethylamine (53.7 mg, 531 μmol, 1 eq.) and DCM (20 mL) at 0° C. under N₂. The reaction mixture was stirred for 2 h. LCMS indicated the SM was consumed, the reaction was clear. The reaction mixture was poured into water (20 mL). The aqueous layer was extracted with DCM (20 mL) 3 times. The combined organic layer was washed with brine and dried over Na₂SO₄. The product did not require further purification. The product 1′-(tetrahydro-2H-pyran-4-carbonyl)spiro[indoline-3,3′-pyrrolidin]-2-one 30.2 (230 mg, 719 μmol) was obtained. LCMS: 301.1 [M+H]⁺.

### Step-2: Procedure for Synthesis of 1′-((tetrahydro-2H-pyran-4-yl)methyl)spiro[indoline-3,3′-pyrrolidine] 30.3

A 50 mL round-bottomed flask was charged with 1′-(tetrahydro-2H-pyran-4-carbonyl)spiro[indoline-3,3′-pyrrolidin]-2-one 30.2 200 mg, 665 μmol, 1 eq.) and 1 M trifluoro(oxolan-1-ium-1-yl)boranuide (20 mL, 20.0 mmol, 30 eq.) under N₂. The reaction mixture was stirred at 70° C. for 8 h. LCMS indicated the SM was consumed. The reaction was quenched with MeOH (1 mL). The reaction mixture was poured into water (20 mL). The pH was adjusted to 8-9 with solid NaHCO₃. The aqueous layer was extracted with EA (20 mL) 3 times. The combined organic layer was washed with brine and dried over Na₂SO₄. The solvent was removed under vacuum. The residue was purified by flash column chromatography (MeOH in DCM from 2% to 10%) to afford 1′-((tetrahydro-2H-pyran-4-yl)methyl)spiro[indoline-3,3′-pyrrolidine] 30.3 (95.0 mg, 348 μmol) of the product as a pale-yellow solid. LCMS: 273.0 [M+H]⁺.

### Step-3: Procedure for Synthesis of N,N-dimethyl-4-((1′-((tetrahydro-2H-pyran-4-yl)methyl)spiro[indoline-3,3′-pyrrolidin]-1-yl)sulfonyl)benzenesulfonamide

A 50 mL round-bottomed flask was charged with 1′-((tetrahydro-2H-pyran-4-yl)methyl)spiro[indoline-3,3′-pyrrolidine] 30.3 (80 mg, 293 μmol, 1 eq.), 4-(dimethylsulfamoyl)benzene-1-sulfonyl chloride (83.1 mg, 293 μmol, 1 eq.), triethylamine (59.2 mg, 586 μmol, 2 eq.) and DCM (10 mL) at room temperature. The reaction mixture was stirred for 2 h when LCMS revealed the starting material completely consumed. The reaction mixture was poured into H₂O (20 mL). The aqueous layer was extracted with DCM (25 mL) 3 times. The organic layers were combined and washed with brine and dried over Na₂SO₄. The crude product was purified by prep-HPLC to give N,N-dimethyl-4-({1′-[(oxan-4-yl)methyl]-1,2-dihydrospiro[indole-3,3′-pyrrolidin]-1-yl}sulfonyl)benzene-1-sulfonamide, I-96.

Yield: 31.4 mg, 20.5%; Appearance: White solid; ¹H NMR (400 MHz, CDCl3) δ 7.96 (d, J=8.3 Hz, 2H), 7.85 (d, J=8.4 Hz, 2H), 7.64 (d, J=8.1 Hz, 1H), 7.24 (d, J=7.8 Hz, 1H), 7.20 (d, J=7.0 Hz, 1H), 7.08 (d, J=7.4 Hz, 1H), 4.04-3.89 (m, 3H), 3.72 (d, J=10.6 Hz, 1H), 3.38 (q, J=10.2 Hz, 2H), 2.73 (s, 6H), 2.57 (s, 1H), 2.36 (s, 2H), 2.27 (s, 2H), 1.94 (s, 4H), 1.67 (m, 2H); HPLC purity: 100.00%; LCMS calculated for C₂₅H₃₃N₃O₅S₂: 519.19; Observed: 520.2 [M+H]⁺.

### Example 31—Synthesis of 4-{[1′-(2,2-difluoropropyl)-1,2-dihydrospiro[indole-3,4′-piperidin]-1-yl]sulfonyl}-N,N-dimethylbenzene-1-sulfonamide (I-97)

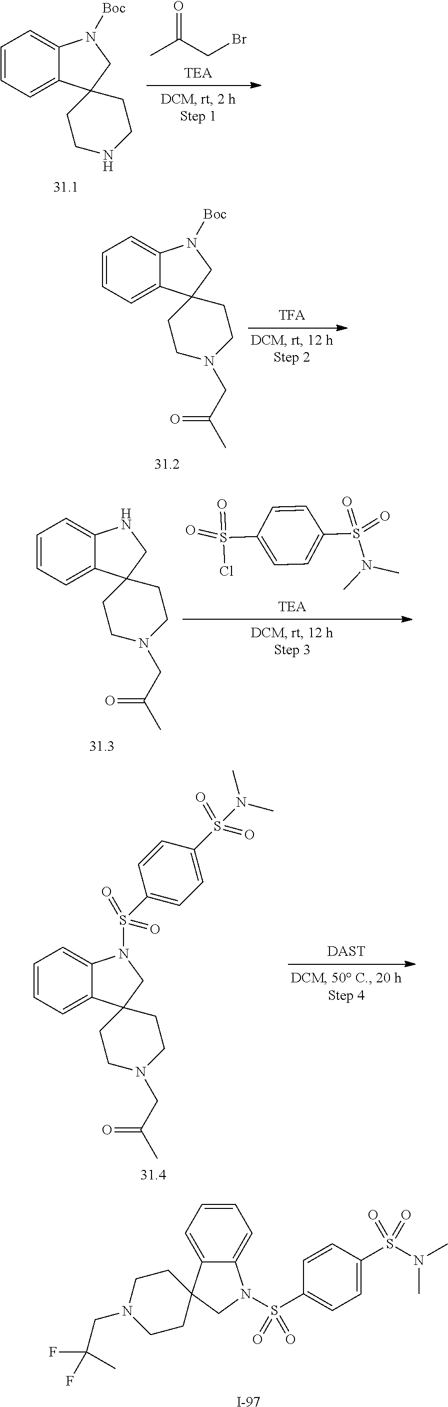

### Step-1: Procedure for Synthesis of tert-butyl 1′-(2-oxopropyl)spiro[indoline-3,4′-piperidine]-1-carboxylate 31.2

To the mixture of tert-butyl 1,2-dihydrospiro[indole-3,4′-piperidine]-1-carboxylate 31.1 (HCl salt, 650 mg, 1.85 mmol, 1.0 eq.), TEA (935 mg, 9.25 mmol, 5.0 eq.) in DCM (10 mL) was added 1-bromopropan-2-one (260 mg, 1.90 mmol, 1.0 eq.) drop-wise at 25° C. The resulting mixture was stirred at 70° C. for 1.5 h before poured into water (30 mL). The mixture was extracted with EA (50 mL×3). The combined organic phase was dried over Na₂SO₄ and concentrated in vacuo to get crude product tert-butyl 1′-(2-oxopropyl)-1,2-dihydrospiro[indole-3,4′-piperidine]-1-carboxylate 31.2 (598 mg, 1.73 mmol, 91.4%) as a yellow sticky oil. LCMS: 345.4 [M+H]⁺.

### Step-2: Procedure for Synthesis of 1-(spiro[indoline-3,4′-piperidin]-1′-yl)propan-2-one 31.3

A mixture of tert-butyl 1′-(2-oxopropyl)spiro[indoline-3,4′-piperidine]-1-carboxylate 31.2 (598 mg, 1.73 mmol, 1.0 eq.), 2,2,2-trifluoroacetaldehyde (55.2 mg, 564 umol, 0.32 eq.) in DCM (10 mL) at room temperature for 2 h. The reaction solution was concentrated to get crude product 1-(spiro[indoline-3,4′-piperidin]-1′-yl)propan-2-one 31.3 (1.2 g, TFA salt, purity: 49.3%) as a brown oil. LCMS: 246.2 [M+H]⁺.

### Step-3: Procedure for Synthesis of N,N-dimethyl-4-((1′-(2-oxopropyl)spiro[indoline-3,4′-piperidin]-1-yl)sulfonyl)benzenesulfonamide 31.4

The mixture of 1-{1,2-dihydrospiro[indole-3,4′-piperidin]-1′-yl}propan-2-one 31.3 (406 mg, TFA salt, purity: 49.3%, 818 umol, 1.0 eq.), 4-(dimethylsulfamoyl)benzene-1-sulfonyl chloride (232 mg, 818 umol, 1.0 eq.) and TEA (247 mg, 2.45 mmol, 3.0 eq.) in DCM (10 mL) was stirred at 25° C. for 2 h. LCMS indicated that the reaction was completed. Then, the reaction was quenched with saturated sodium bicarbonate, extracted with DCM (3×10 mL). The combined organic layer was washed by saturated brine, dried over Na₂SO₄, filtered, concentrated to dryness. The resulting residue was purified by column chromatography on silica gel (PE/EA=2/1) to afford N,N-dimethyl-4-((1′-(2-oxopropyl)spiro[indoline-3,4′-piperidin]-1-yl)sulfonyl)benzenesulfonamide 31.4 (110 mg, purity: 90%, 223 μmol) as a white solid. LCMS: 492.3 [M+H]⁺.

### Step-4: Procedure for Synthesis of 4-((1′-(2,2-difluoropropyl)spiro[indoline-3,4′-piperidin]-1-yl)sulfonyl)-N,N-dimethylbenzenesulfonamide

To the mixture of N,N-dimethyl-4-({[1′-(2-oxopropyl)-1,2-dihydrospiro[indole-3,4′-piperidin]-1-yl]sulfonyl}benzene-1-sulfonamide 31.4 (110 mg, 223 μmol) in DCM (5 mL) add DAST (178 mg, 1.11 mmol, 5.0 eq.) at 50° C. for 20 h. Then, the reaction was added water (20 mL), extracted with DCM (3×30 mL), combined organic layers, washed by saturated brine, dried over Na₂SO₄, filtered, concentrated to dryness. The resulting residue was purified by Pre-HPLC to afford product 4-({[1′-(2,2-difluoropropyl)-1,2-dihydrospiro[indole-3,4′-piperidin]-1-yl]sulfonyl}-N,N-dimethylbenzene-1 sulfonamide, I-97.

Yield: 20.4 mg, 17.8%; Appearance: White solid; ¹H NMR (400 MHz, CDCl3) δ 7.98 (d, J=8.4 Hz, 2H), 7.85 (d, J=8.4 Hz, 2H), 7.65 (d, J=8.1 Hz, 1H), 7.26-7.19 (m, 1H), 7.14-7.02 (m, 2H), 3.77 (s, 2H), 2.88 (d, J=7.0 Hz, 2H), 2.69 (d, J=20.5 Hz, 8H), 2.32-2.16 (m, 2H), 1.83 (t, J=12.3 Hz, 2H), 1.66 (d, J=18.7 Hz, 3H), 1.24 (d, J=13.2 Hz, 2H); HPLC purity: 100%; LCMS calculated for C₂₃H₂₉F₂N₃O₄S₂: 513.16; Observed: 514.1 [M+H]⁺.

### Example 32—Synthesis of N,N-dimethyl-4-({1′-[(oxan-4-yl)methyl]-1,2-dihydrospiro[indole-3,4′-piperidin]-1-yl}sulfonyl)benzene-1-sulfonamide (I-98)

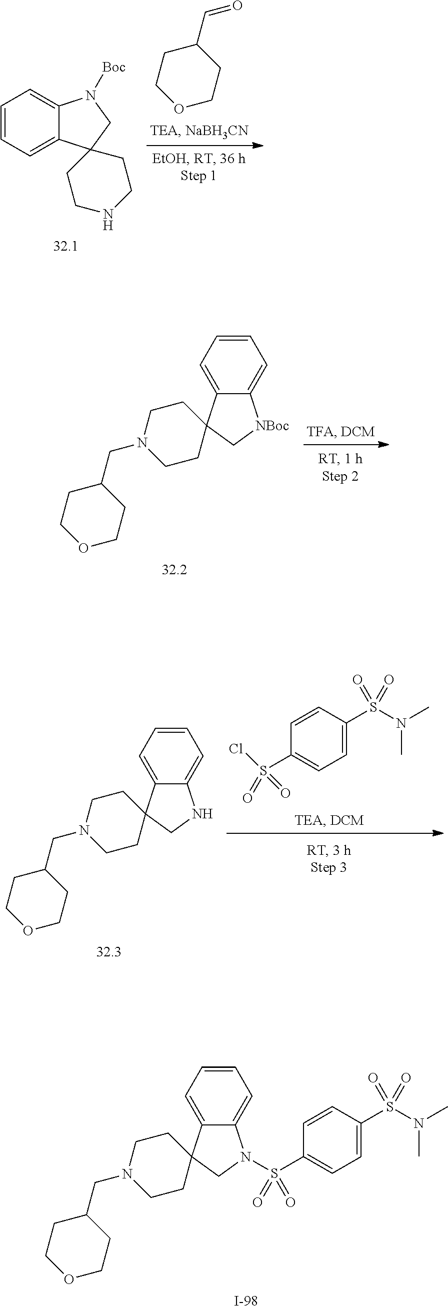

### Step-1: Procedure for Synthesis of tert-butyl 1′-((tetrahydro-2H-pyran-4-yl)methyl)spiro[indoline-3,4′-piperidine]-1-carboxylate 32.2

To a solution of tert-butyl 1,2-dihydrospiro[indole-3,4′-piperidine]-1-carboxylate 32.1 (200 mg, 693 μmol, 1 eq.) and oxane-4-carbaldehyde (157 mg, 1.38 mmol, 2 eq.) in ethyl alcohol (10 mL) was triethylamine (70.1 mg, 693 μmol, 1 eq.). After the mixture was stirred at RT for 6 hours, boron (3⁺) sodium iminomethanide trihydride (217 mg, 3.46 mmol, 5 eq.) was added and the resulting mixture was stirred for 12 h. The mixture was poured into water (20 mL). The aqueous layer was extracted with EA (20 mL) 3 times. The combined organic layer was washed with brine and dried over Na₂SO₄. The solvent was removed in vacuo. The product tert-butyl 1′-[(oxan-4-yl)methyl]-1,2-dihydrospiro[indole-3,4′-piperidine]-1-carboxylate (338 mg, 875 μmol) 32.2 was obtained without further purification. LCMS: 387.3 [M+H]⁺.

### Step-2: Procedure for Synthesis of 1′-((tetrahydro-2H-pyran-4-yl)methyl)spiro[indoline-3,4′-piperidine] 32.3

A 50 mL round-bottomed flask was charged with tert-butyl 1′-[(oxan-4-yl)methyl]-1,2-dihydrospiro[indole-3,4′-piperidine]-1-carboxylate 32.2 (268 mg, 693 μmol, 1 eq.), trifluoroacetic acid (790 mg, 6.93 mmol, 10 eq.) and methylene chloride (15 mL). The reaction mixture was stirred at room temperature for 1 h. LCMS indicated the SM was completely consumed. The reaction mixture was concentrated to dryness. The pH of mixture was adjusted to 8-9 with aqueous NaHCO₃. The aqueous layer was extracted with DCM (20 mL) 3 times. The combined organic layer was washed with brine and dried over Na₂SO₄. No further purification was required. The product 1′-((tetrahydro-2H-pyran-4-yl)methyl)spiro[indoline-3,4′-piperidine] 32.3 (149 mg, 520 μmol) was obtained. LCMS: 287.2 [M+H]⁺.

### Step-3: Procedure for Synthesis of N,N-dimethyl-4-((1′-((tetrahydro-2H-pyran-4-yl)methyl)spiro[indoline-3,4′-piperidin]-1-yl)sulfonyl)benzenesulfonamide

A 50 mL round-bottomed flask was charged with 1′-[(oxan-4-yl)methyl]-1,2-dihydrospiro[indole-3,4′-piperidine] (181.9 mg, 631 μmol), 4-(dimethylsulfamoyl)benzene-1-sulfonyl chloride 32.3 (196 mg, 694 μmol, 1 eq.), triethylamine (127 mg, 1.26 mmol, 1.8 eq.) and methylene chloride (15 mL) at RT. The reaction mixture was stirred for 3 h. LCMS indicated the SM was consumed, the reaction was clear. The reaction mixture was poured into water (20 mL). The aqueous layer was extracted with DCM (20 mL) 3 times. The combined organic layer was washed with brine and dried over Na₂SO₄. The crude product was purified by prep-HPLC to give N,N-dimethyl-4-({1′-[(oxan-4-yl)methyl]-1,2-dihydrospiro[indole-3,4′-piperidin]-1-yl}sulfonyl)benzene-1-sulfonamide, I-98.

Yield: 39.3 mg, 11.6%; Appearance: white solid; ¹H NMR (400 MHz, DMSO-d₆) δ 8.06 (d, J=8.6 Hz, 2H), 7.92 (d, J=8.6 Hz, 2H), 7.53 (d, J=8.0 Hz, 1H), 7.35-7.24 (m, 1H), 7.24-7.19 (m, 1H), 7.07 (td, J₁=7.5 Hz, J₂=0.8 Hz, 1H), 3.83 (s, 2H), 3.80 (d, J=2.6 Hz, 2H), 3.31-3.10 (m, 2H), 2.65 (d, J=12.1 Hz, 2H), 2.59 (s, 6H), 2.13 (d, J=7.3 Hz, 2H), 1.91 (t, J=11.5 Hz, 2H), 1.79-1.70 (m, 1H), 1.68-1.62 (m, 2H), 1.58 (d, J=13.1 Hz, 2H), 1.22-1.05 (m, 2H), 1.01 (d, J=12.7 Hz, 2H); HPLC purity: 99.28%; LCMS calculated for C₂₆H₃₅N₃O₅S₂: 533.20; Observed: 534.3 [M+H]⁺.

### Example 33

The following compounds were prepared using standard chemical manipulations and procedures similar to those used for the preparation of Example 32.

| Compound | | |
|---|---|---|
| No. | Structure | Analytical data |
| I-99 | 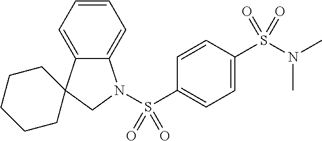 [SMILES: CN(C)S(=O)(=O)c1ccc(S(=O)(=O)N2CC3(CCCCC3)c3ccccc32)cc1] | ¹H NMR (400 MHz, CDCl₃) δ 7.97 (d, J = 8.7 Hz, 2H), 7.83 (d, J = 8.6 Hz, 2H), 7.65 (d, J = 8.1 Hz, 1H), 7.25-7.20 (m, 1H), 7.05 (d, J = 4.2 Hz, 2H), 3.78 (s, 2H), 2.70 (s, 6H), 1.62 (m, 3H), 1.48-1.37 (m, 3H), 1.19-1.28 (m, 5H); HPLC purity: 99.00%; LCMS calculated for C21H26N2O4S2: 434.13; Observed: 435.3 [M + H]⁺. |
| I-121 | 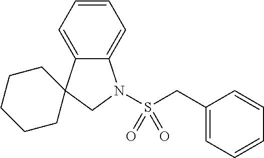 [SMILES: O=S(=O)(Cc1ccccc1)N1CC2(CCCCC2)c2ccccc21] | ¹H NMR (400 MHz, DMSO-d₆) δ 7.30-7.35 (m, 5H), 7.23 (d, J = 7.4 Hz, 1H), 7.09-7.15 (m, 2H), 7.02-6.93 (m, 1H), 4.62 (s, 2H), 3.64 (s, 2H), 1.80-1.38 (m, 7H), 1.15-1.30 (m, 3H); HPLC purity: 99.39%; LCMS calculated for C20H23NO2S: 341.14; Observed: 342.2 [M + H]⁺. |
| I-100 | 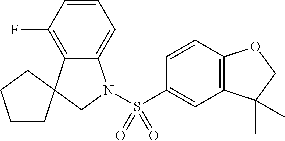 [SMILES: CC1(C)COc2ccc(S(=O)(=O)N3CC4(CCCC4)c4c(F)cccc43)cc21] | ¹H NMR (400 MHz, CDCl₃) δ 7.58 (dd, J = 8.4 Hz, 2.0 Hz, IH), 7.48 (d, J = 1.6 Hz, 1H), 7.44 (dd, J = 8.4 Hz, J = 0.8 Hz, 1H), 7.18-7.12 (m, 1H), 6.78 (d, J = 8.4 Hz, 1H), 6.69-6.64 (m, 1H), 4.29 (s, 2H), 3.65 (s, 2H), 1.98-1.91 (m, 2H), 1.81-1.73 (m, 2H), 1.62-1.59 (m, 2H), 1.44-1.37 (m, 2H), 1.28 (s, 6H); HPLC purity: 100%; LCMS Calculated for C22H24FNO3S: 401.15; Observed: 402.4 [M + H]⁺. |
| I-101 | 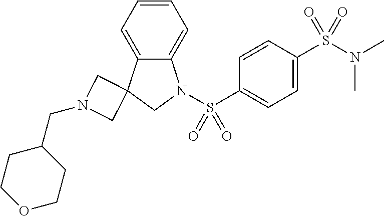 [SMILES: CN(C)S(=O)(=O)c1ccc(S(=O)(=O)N2CC3(CN(CC4CCOCC4)C3)c3ccccc32)cc1] | ¹H NMR (400 MHz, DMSO-d₆) δ 7.96-7.88 (m, 4H), 7.53 (d, J = 6.7 Hz, 1H), 7.49 (d, J = 8.0 Hz, 1H), 7.32 (dd, J = 7.9 Hz, 1.2 Hz, 1H), 7.18 (dd, J = 7.5 Hz, 0.8 Hz, 1H), 4.11 (s, 2H), 3.79 (dd, J = 11.3 Hz, 2.8 Hz, 2H), 3.22 (m, 2H), 3.01 (d, J = 7.0 Hz, 2H), 2.92 (d, J = 7.0 Hz, 2H), 2.60 (s, 6H), 2.22 (d, J = 6.9 Hz, 2H), 1.52 (d, J = 12.8 Hz, 2H), 1.47-1.26 (m, 1H), 1.15-1.04 (m, 2H); HPLC purity: 99.17%; LCMS Calculated for C24H31N3O5S2: 505.17; Observed: 506.1 [M + H]⁺. |
| I-102 | 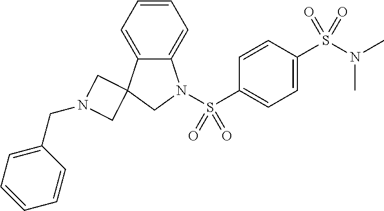 [SMILES: CN(C)S(=O)(=O)c1ccc(S(=O)(=O)N2CC3(CN(Cc4ccccc4)C3)c3ccccc32)cc1] | ¹H NMR (400 MHz, CDCl₃) δ 7.96-7.86 (m, 2H), 7.84-7.77 (m, 2H), 7.64 (d, J = 8.1 Hz, 1H), 7.40-7.28 (m, 7H), 7.15 (d, J = 7.5 Hz, 0.7 Hz, 1H), 4.12 (s, 2H), 3.70 (s, 2H), 3.29 (s, 4H), 2.64 (s, 6H); HPLC purity: 99.45%; LCMS Calculated for C25H27N3O4S2: 497.14; Observed: 498.1 [M + H]⁺. |
| I-122 | 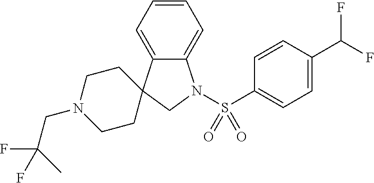 [SMILES: CC(F)(F)CN1CCC2(CC1)CN(S(=O)(=O)c1ccc(C(F)F)cc1)c1ccccc12] | ¹H NMR (400 MHz, CDCl₃) δ 7.92 (d, J = 8.3 Hz, 2H), 7.65 (d, J = 8.1 Hz, 1H), 7.60 (d, J = 8.2 Hz, 2H), 7.23 (d, J = 7.2 Hz, 1H), 7.06 (dd, J = 15.8, 8.4 Hz, 2H), 6.65 (t, J = 55.9 Hz, 1H), 3.76 (s, 2H), 2.86 (s, 2H), 2.68 (s, 2H), 2.24 (s, 2H), 1.81 (s, 2H), 1.67 (t, J = 18.4 Hz, 3H), 1.25 (d, J = 12.9 Hz, 2H); HPLC purity: 99.56 %; LCMS Calculated for C22H24F4N2O2S: 456.15; Observed: 457.1 [M + H]⁺. |
| I-103 | 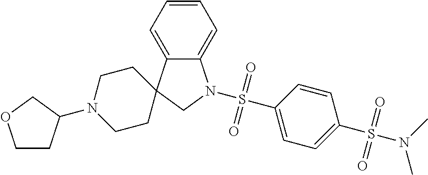 [SMILES: CN(C)S(=O)(=O)c1ccc(S(=O)(=O)N2CC3(CCN(C4CCOC4)CC3)c3ccccc32)cc1] | ¹H NMR (400 MHz, DMSO-d₆) δ 8.06 (d, J = 8.5 Hz, 2H), 7.92 (d, J = 8.5 Hz, 2H), 7.52 (d, J = 8.0 Hz, 1H), 7.34-7.15 (m, 2H), 7.07 (t, J = 8.0 Hz, 1H), 3.84 (s, 2H), 3.81-3.69 (m, 2H), 3.64 (q, J = 7.9 Hz, IH), 3.46 (dd, J = 8.4 Hz, 6.8 Hz, 1H), 2.97-2.85 (m, 1H), 2.72 (d, J = 11.2 Hz, 1H), 2.59 (s, 6H), 2.55 (s, 1H), 2.11-1.86 (m, 3H), 1.76-1.63 (m, 3H), 1.03 (d, J = 13.3 Hz, 2H); HPLC purity: 96.24%; LCMS calculated for C24H31N3O5S2: 505.17; Observed: 506.1 [M + H]⁺. |
| I-104 | 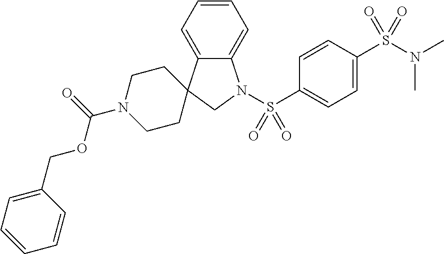 [SMILES: CN(C)S(=O)(=O)c1ccc(S(=O)(=O)N2CC3(CCN(C(=O)OCc4ccccc4)CC3)c3ccccc32)cc1] | 1H NMR (400 MHz, CDCl3) δ 8.00-7.97 (m, 2H), 7.87-7.84 (m, 2H), 7.65 (d, J = 8.0 Hz, 1H), 7.37-7.32 (m, 5H), 7.28-7.24 (m, 1H), 7.09-7.02 (m, 2H), 5.14 (s, 2H), 4.20-4.07 (m, 2H), 3.81 (s, 2H) 2.88-2.80 (m, 2H), 2.70 (s, 6H), 1.77-1.64 (m, 2H), 1.35-1.27 (m, 2H); HPLC purity: 100%; LCMS Calculated for C28H31N3O6S2: 569.17; Observed: 570.1 [M + H]+. |
| I-105 | 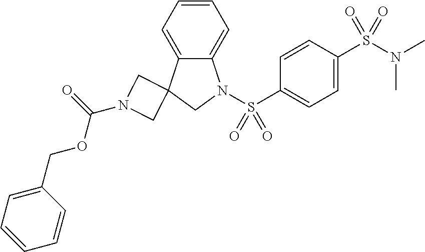 [SMILES: CN(C)S(=O)(=O)c1ccc(S(=O)(=O)N2CC3(CN(C(=O)OCc4ccccc4)C3)c3ccccc32)cc1] | 1H NMR (400 MHz, DMSO-d6) δ 8.03-7.75 (m, 4H), 7.52 (t, J = 6.8 Hz, 2H), 7.43-7.29 (m, 6H), 7.22 (t, J = 7.5 Hz, 1H), 5.03 (s, 2H), 4.24 (s, 2H), 3.84 (s, 2H), 3.67 (s, 2H), 2.55 (s, 6H); HPLC purity: 100.00%; LCMS Calculated for C26H27N3O6S2: 541.13; Observed: 542.0 [M + H]+. |

### Example 34—Synthesis of N,N-dimethyl-4-({6′-methyl-1′,2′-dihydrospiro[cyclohexane-1,3′-pyrazolo[1,5-a]imidazol]-1′-yl}sulfonyl)benzene-1-sulfonamide (I-123)

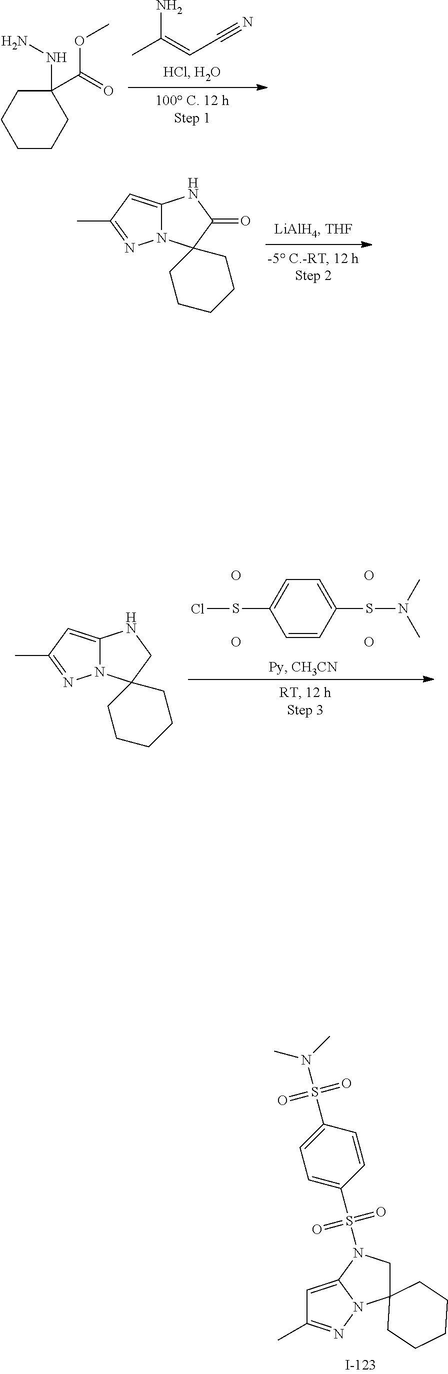

### Step 1. Synthesis of 6′-methylspiro[cyclohexane-1,3′-imidazo[1,2-b]pyrazol]-2′(1′H)-one

(2Z)-3-aminobut-2-enenitrile (0.35 g, 4.25 mmol) was added to a suspension of the methyl 1-hydrazinylcyclohexane-1-carboxylate dihydrochloride (1.04 g, 4.25 mmol) in a 3M HCl/H₂O mixture (⅓, 12 mL), and the resulting mixture was heated under reflux for 12 hours. Then it was cooled to room temperature and neutralized with 2.5M sodium hydroxide aq. solution. The suspension was extracted with dichloromethane (30 mL×3). The combined organic layers were washed with brine (20 mL), dried over sodium sulfate and evaporated in vacuo to give 6′-methylspiro[cyclohexane-1,3′-imidazo[1,2-b]pyrazol]-2′(1′H)-one as a white solid (0.35 g, 1.70 mmol, 100% purity, 40% yield).

### Step 2. Synthesis of 6′-methyl-1′,2′-dihydrospiro[cyclohexane-1,3′-pyrazolo[1,5-a]imidazole]

A solution of 6′-methyl-1′,2′-dihydrospiro[cyclohexane-1,3′-pyrazolo[1,5-a]imidazol]-2′-one (0.35 g, 1.70 mmol) in tetrahydrofuran (25 mL) was added dropwise at −5° C. to a suspension of lithium aluminum hydride (0.079 g, 2.03 mmol) in tetrahydrofuran (5 mL). After addition, the solution was warmed to room temperature and stirred for 12 hours. The solution was quenched with a mixture of water/tetrahydrofuran (1/1, 5 mL). The resulting mixture was filtered and the filtrate evaporated under reduce pressure to give 6′-methyl-1′,2′-dihydrospiro[cyclohexane-1,3′-pyrazolo[1,5-a]imidazole] (0.11 g, 0.575 mmol, 100% purity, 33.8% yield).

### Step 3. Synthesis of N,N-dimethyl-4-({6′-methyl-1′,2′-dihydrospiro[cyclohexane-1,3′-pyrazolo[1,5-a]imidazol]-1′-yl}sulfonyl)benzene-1-sulfonamide

4-(dimethylsulfamoyl)benzene-1-sulfonyl chloride (0.179 g, 0.630 mmol) was added to the mixture of 6′-methyl-1′,2′-dihydrospiro[cyclohexane-1,3′-pyrazolo[1,5-a]imidazole] (0.11 g, 0.575 mmol) and pyridine (0.454 g, 5.75 mmol) in dry acetonitrile (25 mL). The reaction mixture was stirred at room temperature overnight and evaporated. The residue was subjected to HPLC purification (deionized water/HPLC-grade acetonitrile) to afford N,N-dimethyl-4-((6′-methyl-1′,2′-dihydrospiro[cyclohexane-1,3′-pyrazolo[1,5-a]imidazol]-1′-yl sulfonyl)benzene-1-sulfonamide, 1-123. Yield: 33.1 mg, 12.4%; Appearance: White solid; ¹H NMR (600 MHz, DMSO-d₆) δ 8.09 (dd, J=8.6, 2.1 Hz, 2H), 8.01-7.94 (m, 2H), 5.77 (d, J=2.0 Hz, 1H), 4.11 (d, J=2.1 Hz, 2H), 2.60 (d, J=2.0 Hz, 6H), 2.11 (d, J=2.1 Hz, 3H), 1.60 (dt, J=13.8, 4.2 Hz, 2H), 1.51 (ddd, J=15.2, 11.6, 3.8 Hz, 2H), 1.44 (d, J=12.5 Hz, 1H), 1.30-1.21 (m, 2H), 1.16 (d, J=11.5 Hz, 1H), 1.11 (d, J=13.0 Hz, 2H); HPLC purity: 100%; LCMS Calculated for C₁₉H₂₆N₄O₄S₂: 438.56; Observed: 439.2[M+H]⁺.

### Example 35—Synthesis of 4-({1,2′-dihydrospiro[cyclohexane-1,3′-pyrazolo[1,5-a]imidazol]-1′-yl}sulfonyl)-N,N-dimethylbenzene-1-sulfonamide (I-125)

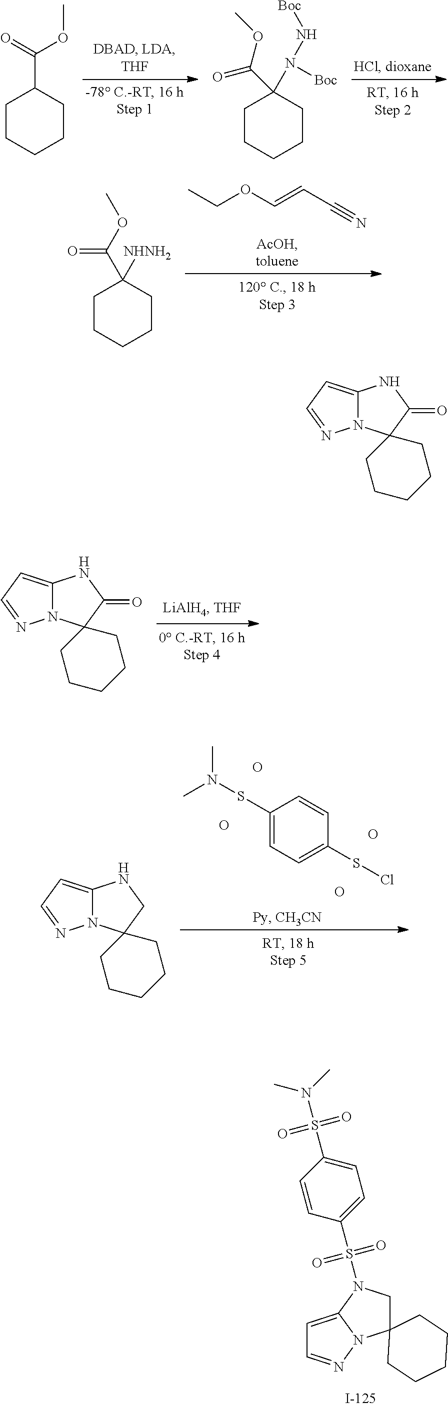

### Step 1. Synthesis of methyl 1-({[(tert-butoxy)carbonyl](([(tert-butoxy)carbonyl]amino))amino}-cyclohexane-1-carboxylate

2.5M n-butyllithium (3.14 g, 49.1 mmol) solution in hexane (19.6 mL) was added dropwise at −10° C. to a stirred solution of bis(propan-2-yl)amine (5.32 g, 52.6 mmol) in dry tetrahydrofuran (500 mL) under argon atmosphere and the reaction mixture was stirred at 0° C. for 0.5 h. Then, the solution of methyl cyclohexanecarboxylate (5 g, 35.1 mmol) in dry tetrahydrofuran (50 mL) was added at −78° C., and the reaction mixture was stirred at −70° C. for 1.5 h. Then, the solution of (E)-N-{[(tert-butoxy)carbonyl]imino}(tert-butoxy)formamide (12.1 g, 52.6 mmol) in dry tetrahydrofuran (50 mL) was added at −78° C., after the reaction mixture was allowed to warm up and stir overnight at room temperature. Then, it was poured in water (500 mL) and extracted with ethyl acetate (250 mL×3). The organic layer was washed with water (250 mL), brine (250 mL), dried over anhydrous sodium sulfate, filtered and concentrated under reduced pressure. Methyl 1-([(tert-butoxy)carbonyl]({[(tert-butoxy)carbonyl]amino)amino}cyclohexane-1-carboxylate was obtained as beige oil (10 g, 26.8 mmol, 80% purity, 61.5% yield) and used in the next step without further purification.

### Step 2. Synthesis of methyl 1-hydrazinylcyclohexane-1-carboxylate dihydrochloride

Methyl 1-({[(tert-butoxy)carbonyl]({[(tert-butoxy)carbonyl]amino})amino}cyclohexane-1-carboxylate (10 g, 21.4 mmol) was added to a stirred HCl sat. solution in dry dioxane (200 mL). The reaction mixture was stirred at room temperature for 18 h. The formed solid was filtered and washed with MTBE (50 mL×3) that afforded methyl 1-hydrazinylcyclohexane-1-carboxylate dihydrochloride as white solid (3 g, 12.2 mmol, 95% purity, 54.3% yield).

### Step 3. Synthesis of 1′,2′-dihydrospiro[cyclohexane-1,3′-pyrazolo[1,5-a]imidazol]-2′-one

Methyl 1-hydrazinylcyclohexane-1-carboxylate dihydrochloride (1 g, 4.07 mmol) and (2E)-3-ethoxyprop-2-enenitrile (0.473 g, 4.07 mmol) were added to acetic acid (50 mL). The reaction mixture was stirred at 120° C. for 18 h, cooled to room temperature, concentrated under reduced pressure. The residue was poured in 10% aq. solution of potassium carbonate (100 mL) and extracted with ethyl acetate (100 mL×3). The organic layer was washed with water (300 mL), brine (300 mL), dried over anhydrous sodium sulfate, filtered and concentrated under reduced pressure. The residue was subjected to HPLC purification (deionized water/HPLC-grade acetonitrile) to afford 1′,2′-dihydrospiro[cyclohexane-1,3′-pyrazolo[1,5-a]imidazol]-2′-one as beige solid (0.2455 g, 1.28 mmol, 95% purity, 29.9% yield).

### Step 4. Synthesis of 1′,2′-dihydrospiro[cyclohexane-1,3′-pyrazolo[1,5-a]imidazole]

1′,2′-dihydrospiro[cyclohexane-1,3′-pyrazolo[1,5-a]imidazol]-2′-one (0.2455 g, 1.28 mmol) was added to slurry of lithium aluminium hydride (0.0651 g, 0.754 mmol) in anhydrous THF (50 mL) at 0° C. and the reaction mixture was allowed to warm up and stir at room temperature for 16 h. Then, it was poured in 10% aq. solution of sodium hydroxide (100 mL) and extracted with ethyl acetate (100 mL×3). The organic layer was washed with water (100 mL), brine (100 mL), dried over anhydrous sodium sulfate, filtered and concentrated under reduced pressure to afford 1′,2′-dihydrospiro[cyclohexane-1,3′-pyrazolo[1,5-a]imidazole] as an beige oil (0.18 g, 0.944 mmol, 100% purity, 79.6% yield).

### Step 5. Synthesis of 4-({1′,2′-dihydrospiro[cyclohexane-1,3′-pyrazolo[1,5-a]imidazol]-1′-yl}sulfonyl)-N,N-dimethylbenzene-1-sulfonamide

Pyridine (0.149 g, 1.88 mmol) and 4-(dimethylsulfamoyl)benzene-1-sulfonyl chloride (0.149 g, 0.525 mmol) were added to a solution of 1′,2′-dihydrospiro[cyclohexane-1,3′-pyrazolo[1,5-a]imidazole] (0.078 g, 0.440 mmol) in acetonitrile (10 mL). The reaction mixture was stirred at room temperature for 18 h. The solvent was removed under reduced pressure and the residue was subjected to HPLC purification (deionized water/HPLC-grade acetonitrile) to afford 4-({1′,2′-dihydrospiro[cyclohexane-1,3′-pyrazolo[1,5-a]imidazol]-1′-yl}sulfonyl)-N,N-dimethylbenzene-1-sulfonamide as a white solid (0.0401 g, 0.0944 mmol, 95% purity, 20.4% yield). The analytical data provided for this compound provisionally supports the proposed structure for 4-({1′,2′-dihydrospiro[cyclohexane-1,3′-pyrazolo[1,5-a]imidazol]-1′-yl}sulfonyl)-N,N-dimethylbenzene-1-sulfonamide, 1-125. Yield: 40.1 mg, 20.4%; Appearance: White solid; ¹H NMR (400 MHz, DMSO-d₆) δ 8.12 (dd, J=8.5, 1.8 Hz, 2H), 8.02-7.92 (m, 2H), 7.41 (d, J=1.9 Hz, 1H), 5.98 (d, J=1.9 Hz, 1H), 4.21 (s, 2H), 2.60 (d, J=1.8 Hz, 6H), 1.54 (tt, J=24.6, 9.5 Hz, 5H), 1.40-1.05 (m, 5H); HPLC purity: 96.75%; LCMS Calculated for C₁₈H₂₄N₄O₄S₂: 424.54; Observed: 425.2[M+H]⁺.

The following example was prepared using standard chemical manipulations and procedures similar to those used for the preparation of the previous example as indicated in the table below:

| Compound | | |
|---|---|---|
| No. | Structure | Analytical Data |
| I-124 | 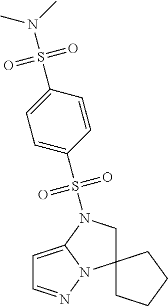 [SMILES: CN(C)S(=O)(=O)c1ccc(S(=O)(=O)N2CC3(CCCC3)n3nccc32)cc1] | Yield: 293.8 mg, 23.3%; Appearance: Orange solid; ¹H NMR (400 MHz, DMSO- d₆) δ 8.08 (d, J = 8.0 Hz, 2H), 7.97 (d, J = 8.3 Hz, 2H), 7.42 (d, J = 1.8 Hz, 1H), 5.99 (d, J = 1.8 Hz, 1H), 4.32 (s, 2H), 2.60 (d, J = 1.6 Hz, 6H), 1.71 (s, 2H), 1.55 (d, J = 14.4 Hz, 6H); HPLC purity: 97.3%; LCMS Calculated for C₁₇H₂₂N₄O₄S₂: 410.51; Observed: 411.2[M + H]⁺. |

### Example 36—Synthesis of N,N-dimethyl-4-((1-(2,2,2-trifluoroethyl)-2′,3′-dihydro-1′H-spiro[piperidine-4,4′-quinolin]-1′-yl)sulfonyl)benzenesulfonamide (I-126)

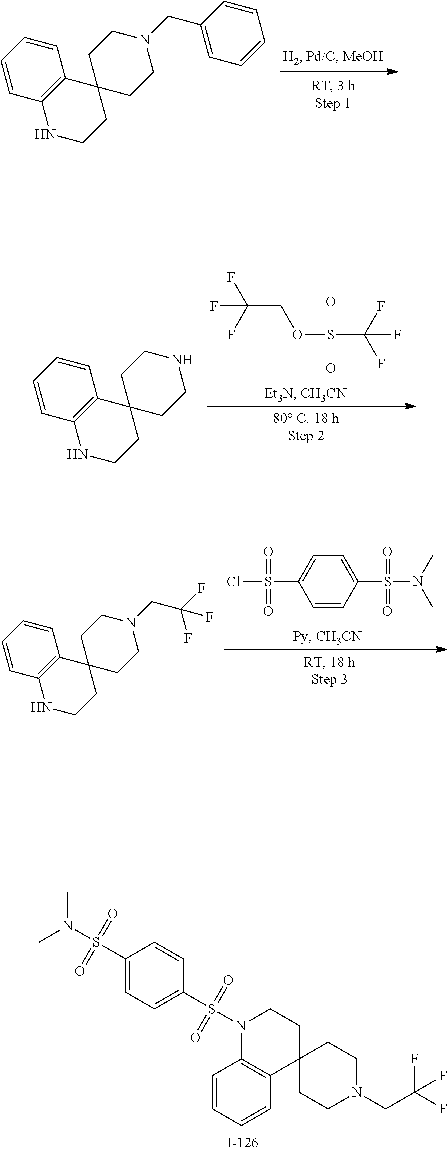

### Step 1. Synthesis of 2′,3′-dihydro-1′H-spiro[piperidine-4,4′-quinoline]

10% Pd/C (0.2 g) was added to the solution of 1-benzyl-2′,3′-dihydro-1′H-spiro[piperidine-4,4′-quinoline] (0.8 g, 2.73 mmol) in methanol (50 mL) and the reaction mixture was hydrogenated at ambient pressure and room temperature until reaction completion (3 h). After the mixture was filtered, formed precipitate washed with methanol (50 mL). Combined filtrates were concentrated under reduced pressure to afford 2′,3′-dihydro-1′H-spiro[piperidine-4,4′-quinoline] as colorless oil (0.453 g, 2.3 mmol, 91% purity, 74.6% yield) that was used in next step without further purification.

### Step 2. Synthesis of 1-(2,2,2-trifluoroethyl)-2′,3′-dihydro-1′H-spiro[piperidine-4,4′-quinoline]

2,2,2-trifluoroethyl trifluoromethanesulfonate (0.568 g, 2.45 mmol) was added to a solution of 2′,3′-dihydro-1′H-spiro[piperidine-4,4′-quinoline] (0.453 g, 2.23 mmol) and triethylamine (0.337 g, 3.34 mmol) in acetotitrile (15 mL). The mixture was stirred at 80° C. for 18 h, cooled to room temperature, and filtered. The filtrate was evaporated under reduced pressure to give 1-(2,2,2-trifluoroethyl)-2′,3′-dihydro-1′H-spiro[piperidine-4,4′-quinoline] (0.58 g, 2.03 mmol, 91% purity, 83.1% yield). The analytical data provided for this compound provisionally supports the proposed structure for 1-(2,2,2-trifluoroethyl)-2′,3′-dihydro-1′H-spiro[piperidine-4,4′-quinoline].

### Step 3. Synthesis of N,N-dimethyl-4-((1-(2,2,2-trifluoroethyl)-2′,3′-dihydro-1′H-spiro[piperidine-4,4′-quinolin]-1′-yl)sulfonyl)benzenesulfonamide

Pyridine (192 mg, 2.43 mmol) and 4-(dimethylsulfamoyl)benzene-1-sulfonyl chloride (632 mg, 2.23 mmol) were added to a solution of 1-(2,2,2-trifluoroethyl)-2′,3′-dihydro-1′H-spiro[piperidine-4,4′-quinoline] (0.58 g, 2.03 mmol) in acetonitrile (25 mL). The reaction mixture was stirred at room temperature for 18 h until completion (TLC control) and the solvent was removed under reduced pressure. The residue was subjected to HPLC purification (deionized water/HPLC-grade acetonitrile) that afforded N,N-dimethyl-4-((1-(2,2,2-trifluoroethyl)-2′,3′-dihydro-1′H-spiro[piperidine-4,4′-quinolin]-1′-yl)sulfonyl)benzenesulfonamide, I-126. Yield: 46.8 mg, 4.37%; Appearance: Yellow solid; ¹H NMR (400 MHz, DMSO-d₆) δ 7.90 (d, J=8.3 Hz, 2H), 7.77 (d, J=8.2 Hz, 2H), 7.69-7.56 (m, 1H), 7.50-7.38 (m, 1H), 7.25 (hept, J=5.5 Hz, 2H), 3.77 (dd, J=7.6, 3.9 Hz, 2H), 3.14 (q, J=10.3 Hz, 2H), 2.56 (d, J=15.5 Hz, 8H), 2.38 (t, J=12.0 Hz, 2H), 1.81 (td, J=13.1, 4.4 Hz, 2H), 1.42 (t, J=5.8 Hz, 2H), 0.93 (d, J=13.2 Hz, 2H); HPLC purity: 100%: LCMS Calculated for C₂₃H₂₈F₃N₃O₄S₂: 431.61; Observed: 432.0[M+H]⁺.

### Example 37—Synthesis of 4-({1′,2′-dihydrospiro[cyclopentane-1,3′-pyrrolo[2,3-c]pyridin]-1′-yl}sulfonyl)-N,N-dimethylbenzene-1-sulfonamide (I-127)

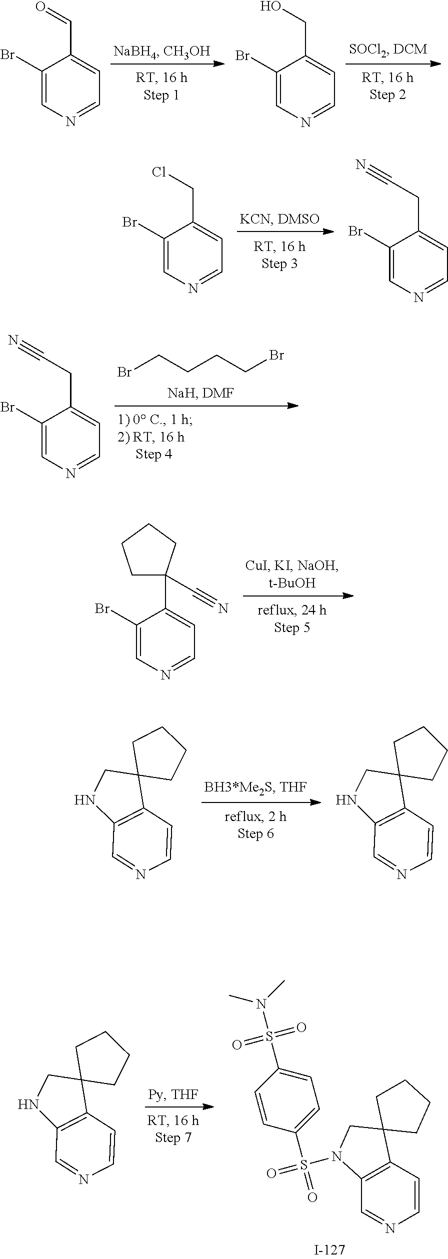

### Step 1. Synthesis of (3-bromopyridin-4-yl)methanol

Sodium borohydride (2.53 g, 67 mmol) was added portionwise to a solution of 3-bromopyridine-4-carbaldehyde (25 g, 134 mmol) in methanol (200 mL). The reaction mixture was stirred overnight at room temperature and the solvent was evaporated under reduced pressure. The residue was partitioned between water/ethyl acetate (200 mL/200 mL). The organic layer was separated, dried over sodium sulfate, filtered and evaporated under reduced pressure to afford (3-bromopyridin-4-yl)methanol (17 g, 90.4 mmol, 95% purity, 64.1% yield).

### Step 2. Synthesis of 3-bromo-4-(chloromethyl)pyridine

Thionyl chloride (12.8 g, 108 mmol) was added dropwise to a solution of (3-bromopyridin-4-yl)methanol (17 g, 90.4 mmol) in dichloromethane (30 mL) and the mixture was stirred overnight at room temperature. After reaction completion (NMR control) the mixture was washed with NaHCO₃ aq. sat. solution, concentrated in vacuum to afford 3-bromo-4-(chloromethyl)pyridine (12 g, 58.1 mmol, 64.5% yield) that was used in the next step immediately without further purification.

### Step 3. Synthesis of 2-(3-bromopyridin-4-yl)acetonitrile

Potassium cyanide (5.67 g, 87.1 mmol) was added to a solution of 3-bromo-4-(chloromethyl)pyridine (12 g, 58.1 mmol) in DMSO (50 mL), the mixture was stirred overnight and diluted with water (100 mL). The product was extracted with ethylacetate (50 mL×2), combined organic layers were dried over sodium sulfate, filtered and evaporated under reduced pressure to afford crude product which was purified by flash chromatography (chloroform/MTBE) to give 2-(3-bromopyridin-4-yl)acetonitrile (8 g, 40.6 mmol, 95% purity, 66.6% yield).

### Step 4. Synthesis of 1-(3-bromopyridin-4-yl)cyclopentane-1-carbonitrile

2-(3-bromopyridin-4-yl)acetonitrile (8 g, 40.6 mmol) was added portionwise to the suspension of sodium hydride (1.62 g, 40.6 mmol, 60 w %) in DMF (20 mL) at 0° C. The mixture was stirred 30 min at this temperature and 1,4-dibromobutane (8.76 g, 40.6 mmol) was added portionwise at the same temperature. After the mixture was allowed to warm up to room temperature and stirred overnight until completion. The solvent was evaporated under reduced pressure, the residue was treated with water/ethyl acetate mixture (30 mL/30 mL). The organic layer was separated, dried over sodium sulfate, filtered and evaporated under reduced pressure. The crude product was purified by flash chromatography (hexane/MTBE) to afford 1-(3-bromopyridin-4-yl)cyclopentanecarbonitrile (4 g, 15.9 mmol, 95% purity, 37.6% yield).

### Step 5. Synthesis of spiro[cyclopentane-1,3′-pyrrolo[2,3-c]pyridin]-2′(1′H)-one

Potassium iodide (0.066 g, 0.398 mmol), copper iodide (0.076 g, 0.398 mmol), and N-acetylglycine (0.047 g, 0.398 mmol) were added to a solution of 1-(3-bromopyridin-4-yl)cyclopentanecarbonitrile (1 g, 3.98 mmol) and sodium hydroxide (0.475 g, 11.9 mmol) in tert-butanol (20 mL). The mixture was refluxed for 24 h, then cooled to room temperature, filtered through silica, and the filtrate was evaporated to dryness. The residue was treated with water/ethyl acetate mixture (50 mL/50 mL). The organic layer was separated, dried over sodium sulfate, filtered and evaporated under reduced pressure. The crude product was purified by flash chromatography (chloroform/MTBE) to afford spiro[cyclopentane-1,3′-pyrrolo[2,3-c]pyridin]-2′(1′H)-one (0.4 g, 2.12 mmol, 95% purity, 50.7% yield).

### Step 6. Synthesis of 1′,2′-dihydrospiro[cyclopentane-1,3′-pyrrolo[2,3-c]pyridine

10 M dimethylsulfide borane complex solution in THF (0.636 mL, 0.483 g, 6.36 mmol) was added to a solution of spiro[cyclopentane-1,3′-pyrrolo[2,3-c]pyridin]-2′(1′H)-one (0.4 g, 2.12 mmol) in dry THF (20 mL), the mixture was refluxed for 2 h and cooled to room temperature. Then methanol (10 mL) was added dropwise, the mixture was refluxed for 2 h, cooled to room temperature and evaporated under reduced pressure. The residue was treated with water/ethyl acetate mixture (20 mL/20 mL). The organic layer was separated, dried over sodium sulfate, filtered and evaporated under reduced pressure to afford crude 1′,2′-dihydrospiro[cyclopentane-1,3′-pyrrolo[2,3-c]pyridine] (0.2 g, 1.14 mmol, 50% purity, 27.1% yield) that was used in the next step without further purification.

### Step 7. Synthesis of 4-({1′,2′-dihydrospiro[cyclopentane-1,3′-pyrrolo[2,3-c]pyridin]-1′-yl}sulfonyl)-N,N-dimethylbenzene-1-sulfonamide

4-(N,N-dimethylsulfamoyl)benzene-1-sulfonyl chloride (0.323 g, 1.14 mmol) was added to the mixture of 1′,2′-dihydrospiro[cyclopentane-1,3′-pyrrolo[2,3-c]pyridine] (0.2 g, 1.14 mmol) and pyridine (0.135 g, 1.71 mmol) in dry THF (20 mL). The reaction mixture was stirred overnight and evaporated. The residue was subjected to HPLC purification (deionized water/HPLC-grade acetonitrile) that afforded 4-({1′,2′-dihydrospiro[cyclopentane-1,3′-pyrrolo[2,3-c]pyridin]-1′-yl}sulfonyl)-N,N-dimethylbenzene-1-sulfonamide, 1-127. Yield: 21.5 mg, 4.24%; Appearance: Yellow solid; ¹H NMR (400 MHz, DMSO-d₆) δ 8.70 (s, 1H), 8.30 (d, J=4.9 Hz, 1H), 8.09 (dd, J=8.5, 1.9 Hz, 2H), 7.98-7.89 (m, 2H), 7.30 (d, J=4.9 Hz, 1H), 3.80 (s, 2H), 2.59 (s, 6H), 1.81-1.58 (m, 4H), 1.54 (q, J=5.8, 5.0 Hz, 2H), 1.41 (dd, J=11.4, 6.2 Hz, 2H); HPLC purity: 100%; LCMS Calculated for C₁₉H₂₃N₃O₄S₂: 421.53; Observed: 422.2[M+H]⁺. 1%

### Example 38—Synthesis of 4-({2′,3′-dihydro-1′H-spiro[cyclohexane-1,4′-quinolin]-1′-yl}sulfonyl)-N,N-dimethylbenzene-1-sulfonamide (I-128)

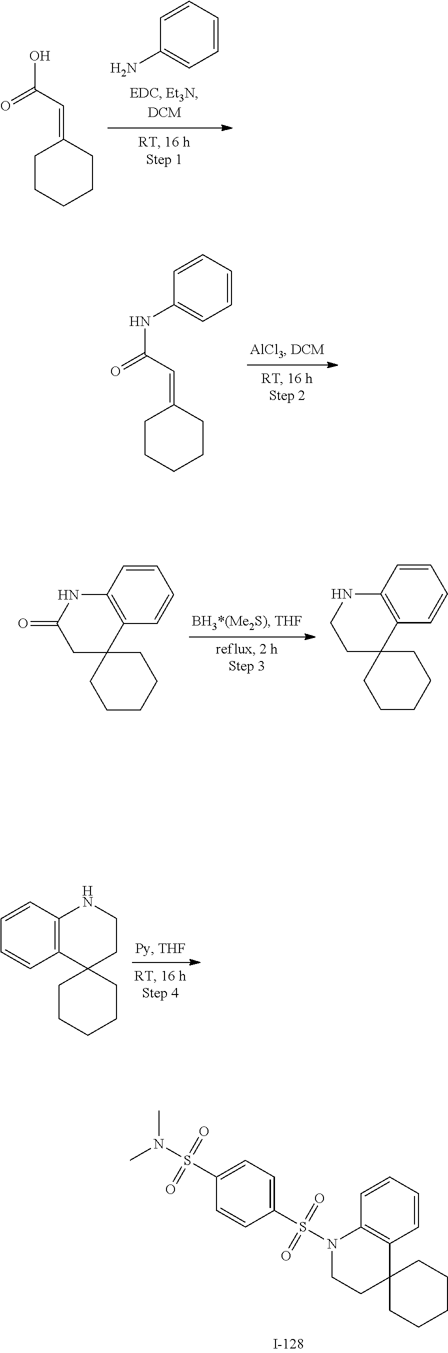

### Step 1. Synthesis of 2-cyclohexylidene-N-phenylacetamide

Triethylamine (5.41 g, 53.5 mmol) was added dropwise at 5° C. to the suspension of 2-cyclohexylideneacetic acid (3 g, 21.4 mmol), aniline (1.99 g, 21.4 mmol), EDC hydrochloride (4.5 g, 23.5 mmol), 1H-1,2,3-benzotriazol-1-ol (3.17 g, 23.5 mmol) in dichloromethane (30 mL). The mixture was stirred overnight, then diluted with water (100 mL). The organic layer was separated, concentrated under reduced pressure. The residue was subjected to flash chromatography (hexane/ethyl acetate) to give 2-cyclohexylidene-N-phenylacetamide (2.1 g, 9.75 mmol, 95% purity, 43.2% yield).

### Step 2. Synthesis of 2′,3′-dihydro-1′H-spiro[cyclohexane-1,4′-quinolin]-2′-one

Trichloroalumane (3.89 g, 29.2 mmol) was added at room temperature to a solution of 2-cyclohexylidene-N-phenylacetamide (2.1 g, 9.75 mmol) in dichloromethane (20 mL), the mixture was stirred overnight. Then water (50 mL) was added to it, the organic layer was separated and concentrated under reduced pressure. Flash chromatography (chloroform/MTBE) of residue resulted in 2′,3′-dihydro-1′H-spiro[cyclohexane-1,4′-quinolin]-2′-one (2.1 g, 9.75 mmol, 90% purity, 90.4% yield).

### Step 3. Synthesis of 2′,3′-dihydro-1′H-spiro[cyclohexane-1,4′-quinoline]

10 M dimethylsulfide borane complex solution in THF (5.1 mL, 3.86 g, 50.9 mmol) was added to a solution of 2′,3′-dihydro-1′H-spiro[cyclohexane-1,4′-quinolin]-2′-one (1.1 g, 5.1 mmol) in dry THF (20 mL). The mixture was refluxed for 2 h and cooled to room temperature. Then methanol (20 mL) was added dropwise, the mixture was refluxed for 2 h, cooled to room temperature and evaporated under reduced pressure. The residue was treated with water/ethyl acetate mixture (20 mL/20 mL). The organic layer was separated, dried over sodium sulfate, filtered and evaporated under reduced pressure to afford crude 2′,3′-dihydro-1′H-spiro[cyclohexane-1,4′-quinoline] (0.8 g, 3.97 mmol, 90% purity, 70.5% yield) that was used in the next step without further purification.

### Step 4. Synthesis of 4-({2′,3′-dihydro-1′H-spiro[cyclohexane-1,4′-quinolin]-1′-yl}sulfonyl)-N,N-dimethylbenzene-1-sulfonamide

4-(N,N-dimethylsulfamoyl)benzene-1-sulfonyl chloride (1.12 g, 3.97 mmol) was added to the mixture of 2′,3′-dihydro-1′H-spiro[cyclohexane-1,4′-quinoline] (0.8 g, 3.97 mmol) and pyridine (0.47 g, 5.95 mmol) in dry THF (20 m|L. The reaction mixture was stirred overnight and evaporated. The residue was subjected to HPLC purification (deionized water/HPLC-grade methanol) that afforded 4-({2′,3′-dihydro-1′H-spiro[cyclohexane-1,4′-quinolin]-1′-yl}sulfonyl)-N,N-dimethylbenzene-1-sulfonamide, I-128. Yield: 110.8 mg, 5.89%; Appearance: Beige solid; ¹H NMR (400 MHz, DMSO-d₆) δ 7.89 (d, J=8.0 Hz, 2H), 7.75 (d, J=8.0 Hz, 2H), 7.68-7.59 (m, 1H), 7.49-7.40 (m, 1H), 7.29-7.14 (m, 2H), 3.76 (t, J=5.5 Hz, 2H), 2.57 (d, J=1.5 Hz, 6H), 1.52 (dd, J=19.5, 7.9 Hz, 3H), 1.37 (t, J=5.7 Hz, 4H), 1.22 (q, J=11.3, 8.9 Hz, 3H), 0.95 (d, J=13.0 Hz, 2H); HPLC purity: 100%; LCMS Calculated for C₂₂H₂₈N₂O₄S₂: 448.6; Observed: 449.0[M+H]⁺.

### Example 39—Synthesis of N,N-dimethyl-4-(spiro[cyclohexane-1,3′-indolin]-1′-ylsulfonyl)benzenesulfonimidamide (I-129)

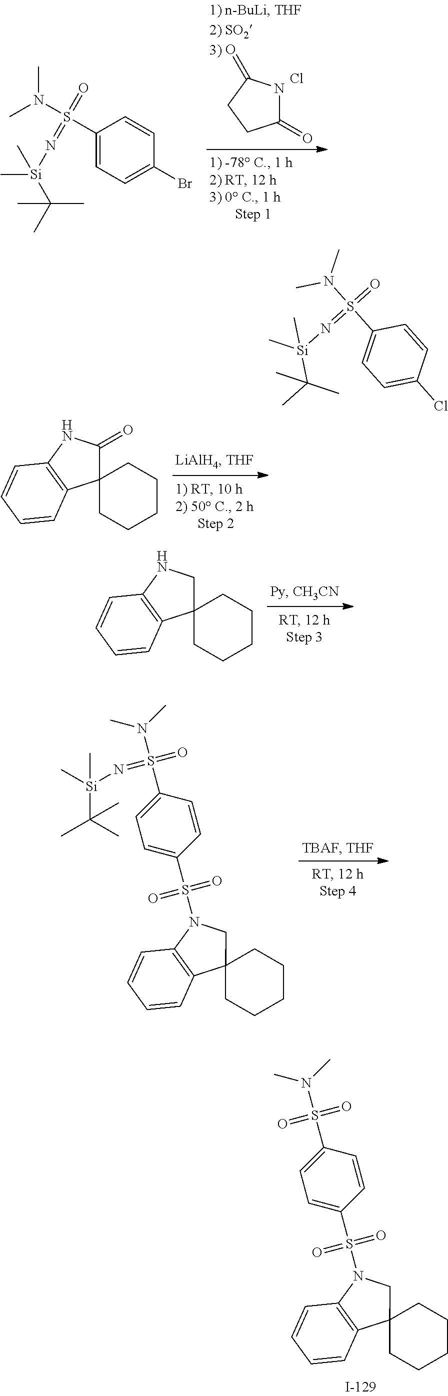

### Step 1. Synthesis of 4-[(tert-butyldimethylsilyl)dimethyl-S-aminosulfonimidoyl]benzene-1-sulfonyl chloride

2.5M n-butyllithium (0.382 g, 5.97 mmol) in hexane (2.38 mL) was added to a solution of 4-bromo-N′-(tert-butyldimethylsilyl)-N,N-dimethylbenzenesulfonimidamide (1.88 g, 4.98 mmol) in anhydrous tetrahydrofuran (20 mL) at −78° C. under argon atmosphere and the mixture was stirred for 1 h at −78° C. After the solution of SO₂ (0.954 g, 14.9 mmol) in tetrahydrofuran (20 mL) was added to the resulting mixture at the same temperature. Then the cooling bath was removed and the mixture was allowed to warm to room temperature and stir for 12 h. The solution was evaporated in vacuo, the residue was dissolved in dichloromethane (20 mL) and N-chlorosuccinimide (0.797 g, 5.97 mmol) was added maintaining the reaction mixture temperature at 0° C. The mixture was stirred for 30 minutes, diluted with ethyl acetate (20 mL) and water (20 mL). The organic layer was separated, dried over sodium sulfate, filtered and the filtrate was evaporated in vacuo to give 4-[(tert-butyldimethylsilyl)dimethyl-S-aminosulfonimidoyl]benzene-1-sulfonyl chloride as a dark resin (1.76 g, 4.43 mmol, 58.66% purity, 52.2% yield) that was used in the next step without further purification.

### Step 2. Synthesis of spiro[cyclohexane-1,3′-indoline]

Lithium aluminium hydride (0.751 g, 19.8 mmol) was added portionwise to a solution of spiro[cyclohexane-1,3′-indolin]-2′-one (2 g, 9.93 mmol) in dry tetrahydrofuran (20 mL). The reaction mixture was stirred at room temperature for 10 h. Then it was heated to 50° C. for 2 h, after cooled to room temperature and quenched with water (2 mL) and 6N NaOH aq. solution (2 mL). The aqueous layer was extracted with ethyl acetate (20 mL-3). The combined organic layers were washed with brine (50 mL), dried over sodium sulfate, filtered and evaporated under reduced pressure to afford spiro[cyclohexane-1,3′-indoline] as white solid (1.8 g, 9.61 mmol, 100% purity, 97.2% yield).

### Step 3. Synthesis of N′-(tert-butyldimethylsilyl)-N,N-dimethyl-4-(spiro[cyclohexane-1,3′-indolin]-1′-ylsulfonyl)benzenesulfonimidamide

4-[(tert-butyldimethylsilyl)dimethyl-S-aminosulfonimidoyl]benzene-1-sulfonyl chloride (1.69 g, 4.27 mmol) was added to the mixture of 1′,2′-dihydrospiro[cyclohexane-1,3′-indole] (0.8 g, 4.27 mmol) and pyridine (0.982 g, 12.4 mmol) in dry acetonitrile (20 mL). The reaction mixture was stirred for 12 h and evaporated in vacuo to give N′-(tert-butyldimethylsilyl)-N,N-dimethyl-4-(spiro[cyclohexane-1,3′-indolin]-1′-ylsulfonyl)benzenesulfonimidamide (1.8 g, approx. 20% purity by NMR, 0.657 mmol, 15.4% yield) as a dark resin, which was used in the next step without purification.

### Step 4. Synthesis of N,N-dimethyl-4-(spiro[cyclohexane-1,3′-indolin]-1′-ylsulfonyl)benzenesulfonimidamide

1M solution TBAF in THF (0.857 g, 3.28 mmol, 3.28 mL) was added to a solution of N-(tert-butyldimethylsilyl)-4-({1′,2′-dihydrospiro[cyclohexane-1,3′-indol]-1′-yl}sulfonyl)-N,N-dimethylbenzene-1-sulfonoimidamide (1.8 g, 3.28 mmol) in dry tetrahydrofuran (50 mL) under argon atmosphere. The reaction mixture was stirred for 12 h at room temperature and evaporated in vacuo. The residue was subjected to HPLC purification (deionized water/HPLC-grade acetonitrile) that afforded N,N-dimethyl-4-(spiro[cyclohexane-1,3′-indolin]-1′-ylsulfonyl)benzenesulfonimidamide, 1-129. Yield: 70.5 mg, 4.71%; Appearance: Orange solid; ¹H NMR (400 MHz, DMSO-d₆) δ 8.00 (d, J=8.4 Hz, 2H), 7.91 (d, J=8.0 Hz, 2H), 7.52 (d, J=8.0 Hz, 1H), 7.25 (t, J=7.7 Hz, 1H), 7.17 (d, J=7.5 Hz, 1H), 7.06 (t, J=7.5 Hz, 1H), 4.70 (s, 1H), 3.81 (s, 2H), 2.50 (s, 6H), 1.35 (dddt, J=70.4, 45.3, 24.2, 12.0 Hz, 8H), 0.96 (t, J=12.4 Hz, 2H); HPLC purity: 100%; LCMS Calculated for C₂₁H₂₆N₃O₃S₂: 433.59; Observed: 434.2[M+H]⁺.

### Example 40—Synthesis of benzyl 1-[4-(difluoromethyl)benzenesulfonyl]-4-fluoro-1,2-dihydrospiro[indole-3,4′-piperidine]-1′-carboxylate (I-130)

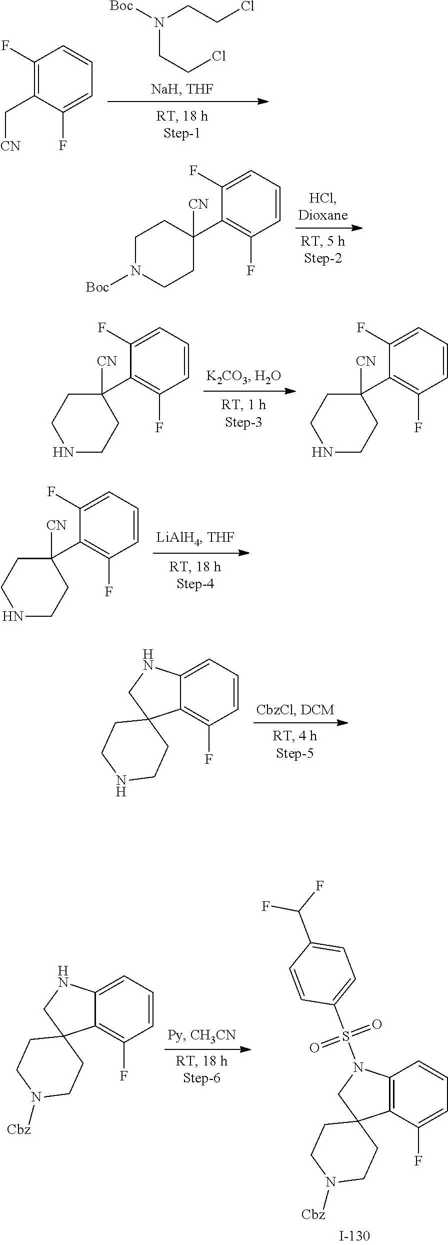

Step-1. Synthesis of tert-butyl 4-cyano-4-(2,6-difluorophenyl)piperidine-1-carboxylate:

2-(2,6-difluorophenyl)acetonitrile (15 g, 97.9 mmol) was added to a slurry of sodium hydride (60% w/w; 9.75 g, 244 mmol) in anhydrous tetrahydrofuran (500 mL) at room temperature over 30 min. The reaction mixture was stirred for 1 h, tert-butyl N,N-bis(2-chloroethyl)carbamate (25.9 g, 107 mmol) was added over 15 min, and the reaction mixture was stirred at room temperature for 18 h. Then, it was poured into water (1000 mL) and extracted with ethyl acetate (250 mL×3). The organic layer was washed with water (300 mL) and brine (300 mL), dried over anhydrous sodium sulfate, filtered, and concentrated under reduced pressure. The residue was subjected to silica gel chromatography purification (hexane/MTBE) to afford tert-butyl 4-cyano-4-(2,6-difluorophenyl)piperidine-1-carboxylate as colorless oil (12 g, 37.2 mmol, 95% purity, 38% yield).

### Step-2. Synthesis of 4-(2,6-difluorophenyl)piperidine-4-carbonitrile hydrochloride

tert-butyl 4-cyano-4-(2,6-difluorophenyl)piperidine-1-carboxylate (5.3 g, 14.6 mmol) was added to a stirred solution of sat. hydrogen chloride solution in dry dioxane (250 mL). The reaction mixture was stirred at room temperature for 18 h. The solid was filtered and washed repeatedly with MTBE, and then air-dried to afford 4-(2,6-difluorophenyl)piperidine-4-carbonitrile hydrochloride as white solid (3.8 g, 14.6 mmol, 100% purity, 89.6% yield).

### Step-3. Synthesis of 4-(2,6-difluorophenyl)piperidine-4-carbonitrile

Potassium carbonate (2.1 g, 14.6 mmol) was added to a stirred solution of 4-(2,6-difluorophenyl)piperidine-4-carbonitrile hydrochloride (3.8 g, 14.6 mmol) in water (250 mL) at 15° C. The mixture was extracted with ethyl acetate (150 mL×3). The organic layer was washed with water (200 mL) and brine (200 mL), dried over anhydrous sodium sulfate, filtered, and concentrated under reduced pressure to give 4-(2,6-difluorophenyl)piperidine-4-carbonitrile as white solid (2.8 g, 12.5 mmol, 95% purity, 82% yield).

### Step-4. Synthesis of 4-fluorospiro[indoline-3,4′-piperidine]

4-(2,6-difluorophenyl)piperidine-4-carbonitrile (1.8 g, 8.09 mmol) was added to a slurry of lithium aluminium hydride (0.820 g, 24.2 mmol) in anhydrous tetrahydrofuran (100 mL) at 0° C. portionwise, and the reaction mixture was warmed to 50° C. and stirred for 18 hours. Then, the reaction mixture was cooled to 0° C., quenched with water (40 mL), and diluted with ethyl acetate (100 mL). The organic layer was separated, dried over anhydrous sodium sulfate, filtered, and concentrated under reduced pressure to afford 4-fluorospiro[indoline-3,4′-piperidine] (0.8 g, 3.29 mmol, 85% purity, 40.9% yield) that was used in the next step without further purification.

### Step-5. Synthesis of benzyl 4-fluoro-1,2-dihydrospiro[indole-3,4′-piperidine]-1′-carboxylate

Triethylamine (0.809 mL, 5.80 mmol) and benzyl carbonochloridate (0.725 g, 4.25 mmol) were added to a solution of 4-fluorospiro[indoline-3,4′-piperidine] (0.8 g, 3.87 mmol) in dichloromethane (50 mL) at room temperature. The mixture was stirred at room temperature for 5 h until completion (TLC control). The reaction mixture was then evaporated under reduced pressure to give benzyl 4-fluoro-1,2-dihydrospiro[indole-3,4′-piperidine]-1′-carboxylate as colorless oil (1.2 g, 3.27 mmol, 93% purity, 84.7% yield) that was used in the next step without further purification.

### Step-6. Synthesis of benzyl 1-[4-(difluoromethyl)benzenesulfonyl]-4-fluoro-1,2-dihydrospiro[indole-3,4′-piperidine]-1′-carboxylate

Pyridine (0.417 g, 5.28 mmol) and 4-(difluoromethyl)benzene-1-sulfonyl chloride (0.417 g, 3.52 mmol) were added to a solution of benzyl 4-fluoro-1,2-dihydrospiro[indole-3,4′-piperidine]-1′-carboxylate (1.2 g, 3.52 mmol) in acetonitrile (100 mL). The reaction mixture was stirred at room temperature for 18 h. The solvent was removed under reduced pressure and the residue was subjected to HPLC purification (deionized water/HPLC-grade acetonitrile) to afford benzyl 1-[4-(difluoromethyl)benzenesulfonyl]-4-fluoro-1,2-dihydrospiro[indole-3,4′-piperidine]-1′-carboxylate (I-130). Yield: 210 mg, 10.6%; Appearance: Beige solid; ¹H NMR (400 MHz, DMSO-d₆) δ 8.02 (d, J=7.4 Hz, 2H), 7.92-7.71 (m, 2H), 7.50-7.13 (m, 8H), 6.93-6.82 (m, 1H), 5.08 (s, 2H), 4.02 (s, 2H), 3.94-3.82 (m, 2H), 3.06-2.82 (m, 2H), 1.89-1.71 (m, 2H), 1.36-1.08 (m, 2H); HPLC purity: 100%; LCMS Calculated for C₂₇H₂₅F₃N₂O₄S: 530.56; Observed: 532.2[M+H]⁺.

### Example 41

The following compound was prepared using standard chemical manipulations and procedures similar to those used for the preparation of the previous example.

| Compound No. | Structure | Analytical Data |
|---|---|---|
| I-131 | 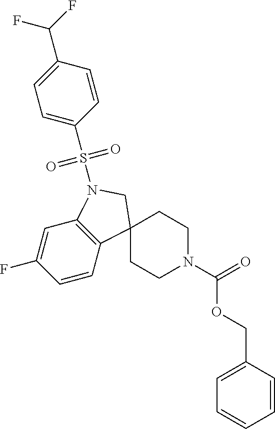 [SMILES: O=C(OCc1ccccc1)N1CCC2(CC1)CN(S(=O)(=O)c1ccc(C(F)F)cc1)c1cc(F)ccc12] | Yield: 19 mg, 4%; Appearance: White solid; ¹H NMR (600 MHz, DMSO-d₆) δ 8.02 (d, J = 8.1 Hz, 2H), 7.78 (d, J = 8.0 Hz, 2H), 7.38-7.27 (m, 5H), 7.27-7.21 (m, 2H), 7.21-7.01 (m, 1H), 6.89-6.82 (m, 1H), 5.12-4.96 (m, 2H), 3.95 (s, 2H), 3.86 (d, J = 13.6 Hz, 2H), 3.04-2.82 (m, 2H), 1.61-1.51 (m, 2H), 1.08 (d, J = 13.2 Hz, 2H).; HPLC purity: 100%; LCMS Calculated for C₂₇H₂₅F₃N₂O₄S: 530.56; Observed: 531.2[M + H]⁺. |

### Example 42—Synthesis of 1-[4-(difluoromethyl)benzenesulfonyl]-5-fluoro-1,2-dihydrospiro[indole-3,4′-piperidine]-1′-carboxylate (I-132)

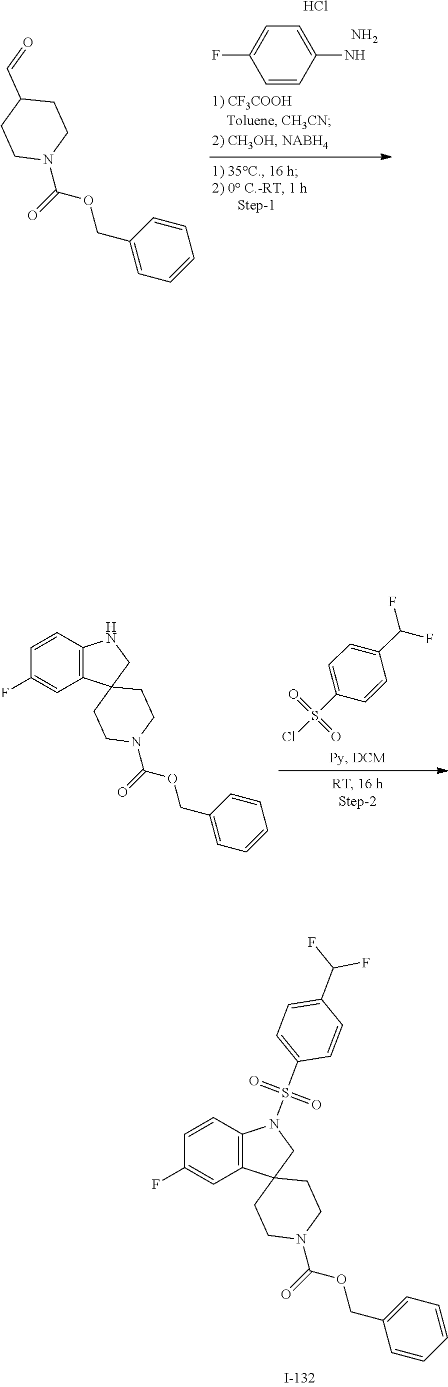

### Step-1. Synthesis of benzyl 5-fluoro-1,2-dihydrospiro[indole-3,4′-piperidine]-1′-carboxylate

A solution of (4-fluorophenyl)hydrazine (3 g, 23.7 mmol) and trifluoroacetic acid (6.0 mL) in a toluene/acetonitrile mixture (49/1, 100 mL) was heated to 35° C., followed by addition dropwise of solution of 4-formyl-piperidine-1-carboxylic acid benzyl ester (5.31 g, 21.5 mmol) in toluene/acetonitrile mixture (49/1, 40 mL). The mixture was stirred at 35° C. for 16 h, then cooled to 0° C. and diluted with cold methanol (10 mL). NaBH₄ (1.22 g, 32.3 mmol) was added slowly to the mixture, and it was stirred for additional 45 min. After, the reaction mixture was washed with aqueous NH₄OH (6%, 50 mL) and brine (100 mL), then dried over sodium sulfate, filtered, and evaporated to dryness. The crude residue was purified by column chromatography (silica gel, ethyl acetate/hexane) to give benzyl 5-fluoro-1,2-dihydrospiro[indole-3,4′-piperidine]-1′-carboxylate as yellow solid (1.75 g, 5.14 mmol, 95% purity, 23.9% yield).

### Step-2. Synthesis of 1-[4-(difluoromethyl)benzenesulfonyl]-5-fluoro-1,2-dihydrospiro[indole-3,4′-piperidine]-1′-carboxylate

4-(Difluoromethyl)benzene-1-sulfonyl chloride (0.0729 g, 322 μmol) was added to a mixture of benzyl 5-fluoro-1,2-dihydrospiro[indole-3,4′-piperidine]-1′-carboxylate (0.1 g, 0.293 mmol) and pyridine (0.0463 g, 0.586 mmol) in dry dichloromethane (25 mL). The reaction mixture was stirred at room temperature overnight and evaporated. The residue was subjected to HPLC purification (deionized water/HPLC-grade acetonitrile) to afford 1-[4-(difluoromethyl)benzenesulfonyl]-5-fluoro-1,2-dihydrospiro[indole-3,4′-piperidine]-1′-carboxylate (I-132). Yield: 82.9 mg, 50.7%; Appearance: Yellow oil solid; ¹H NMR (400 MHz, DMSO-d₆) δ 7.98-7.92 (m, 2H), 7.76 (d, J=8.1 Hz, 2H), 7.52-7.45 (m, 1H), 7.39-7.26 (m, 5H), 7.20-6.99 (m, 3H), 5.05 (d, J=11.4 Hz, 2H), 3.94 (s, 2H), 3.88-3.76 (m, 2H), 2.90 (d, J=53.0 Hz, 2H), 1.64-1.51 (m, 2H), 1.00 (d, J=13.3 Hz, 2H); H PLC purity: 100%; LCMS Calculated for C₂₇H₂₅F₃N₂O₄S: 530.56; Observed: 531.0[M+H]⁺.

### Example 43

The following compound was prepared using standard chemical manipulations and procedures similar to those used for the preparation of the previous example.

| Compound No. | Structure | Analytical Data |
|---|---|---|
| I-133 | 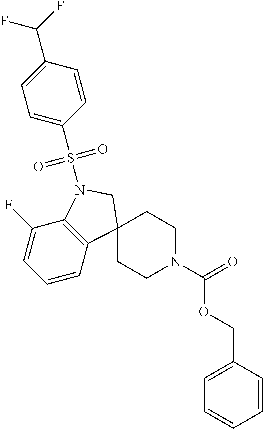 [SMILES: O=C(OCc1ccccc1)N1CCC2(CC1)CN(S(=O)(=O)c1ccc(C(F)F)cc1)c1c(F)cccc12] | Yield: 285.3 mg, 58%; Appearance: Brown oil; ¹H NMR (600 MHz, DMSO-d₆) δ 8.01 (d, J = 8.1 Hz, 2H), 7.80 (d, J = 8.1 Hz, 2H), 7.41-7.27 (m, 5H), 7.24-7.02 (m, 4H), 5.07 (d, J = 7.2 Hz, 2H), 4.18 (s, 2H), 3.93 (d, J = 13.6 Hz, 2H), 3.07- 2.85 (m, 2H), 1.72-1.60 (m, 2H), 1.28 (d, J = 13.2 Hz, 2H).; HPLC purity: 100%; LCMS Calculated for C₂₇H₂₅F₃N₂O₄S: 530.56; Observed: [M + H]⁺. |

### Example 44—Synthesis of benzyl 1′-[4-(difluoromethyl)benzenesulfonyl]-1′,2′-dihydrospiro[piperidine-4,3′-pyrrolo[3,2-b]pyridine]-1-carboxylate (I-134)

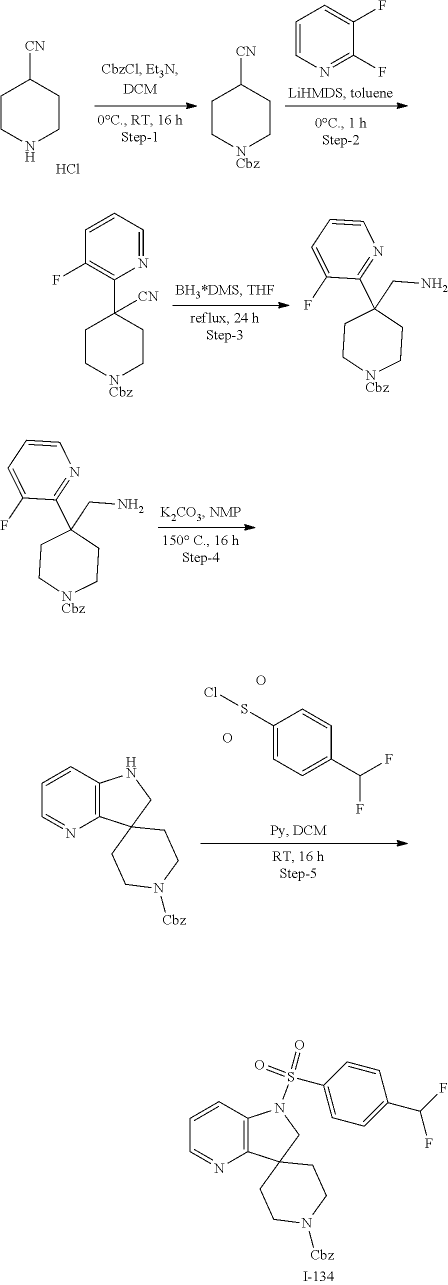

### Step-1. Synthesis of benzyl 4-cyanopiperidine-1-carboxylate

A solution of 4-cyanopiperidine hydrochloride (7.00 g, 47.7 mmol) and triethylamine (6.63 g, 47.7 mmol) in dichloromethane (100 mL) was cooled to 0° C., and benzyl chloroformate (8.52 g, 50.0 mmol) was added dropwise to it. The resulting mixture was warmed to room temperature and stirred for 16 hours. After, the mixture was diluted with water (100 mL) and extracted with dichloromethane (150 mL×2). The combined organic layers were washed with citric acid aq. sat. solution (150 mL) and sodium hydrogen carbonate aq. sat. solution (150 mL), ten dried over sodium sulfate, filtered, and evaporated under reduced pressure to give benzyl 4-cyanopiperidine-1-carboxylate (10 g, 38.8 mmol, 95% purity, 81.8% yield).

### Step-2. Synthesis of benzyl 4-cyano-4-(3-fluoropyridin-2-yl)piperidine-1-carboxylate

Potassium hexamethyldisilazane (37.5 mL, 40.9 mmol, 1.05 M solution in toluene) was slowly added to a solution of 2,3-difluoropyridine (4.70 g, 40.9 mmol) and benzyl 4-cyanopiperidine-1-carboxylate (10 g, 40.9 mmol) in toluene (200 mL) at 0° C. under argon atmosphere. The mixture was stirred at 0° C. for 1 hour. After, the reaction mixture was quenched with 1N HCl aq. solution (100 mL). The mixture was extracted with ethyl acetate (200 mL×2). The combined organic layers were washed with water (200 mL), dried over anhydrous sodium sulfate, filtered, and concentrated in vacuo to give the crude product, which was used without purification.

### Step-3. Synthesis of benzyl 4-(aminomethyl)-4-(3-fluoropyridin-2-yl)piperidine-1-carboxylate

10 M Borane dimethyl sulfide complex solution in tetrahydrofuran (17.2 mL, 172 mmol) was added to a solution of benzyl 4-cyano-4-(3-fluoropyridin-2-yl)piperidine-1-carboxylate (8.4 g, 17.3 mmol) in tetrahydrofuran (100 mL), and the mixture was stirred at reflux for 24 hours. After, the reaction mixture was diluted with methanol (10 mL) and ethyl acetate (200 mL). The organic layer was separated, washed with citric acid aq. sat. solution (100 mL) and sodium bicarbonate aq. sat. solution (100 mL), then dried over sodium sulfate, filtered, and evaporated under reduced pressure to give crude benzyl 4-(aminomethyl)-4-(3-fluoropyridin-2-yl)piperidine-1-carboxylate (8.8 g, 8.84 mmol, 34.5% purity, 51% yield), which was used without further purification.

### Step-4. Synthesis of benzyl 1′,2′-dihydrospiro[piperidine-4,3′-pyrrolo[3,2-b]pyridine]-1-carboxylate

Benzyl 4-(aminomethyl)-4-(3-fluoropyridin-2-yl)piperidine-1-carboxylate (8.8 g, 8.82 mmol) was dissolved in NMP (150 mL). Dipotassium carbonate (12.1 g, 88.2 mmol) was added, and the mixture was heated at 150° C. overnight. After, the reaction mixture was cooled to room temperature. The reaction mixture was washed with citric acid sat. aq. solution (100 mL) and sodium bicarbonate sat. aq. solution (100 mL), then dried over sodium sulfate, filtered, and evaporated under reduced pressure to afford benzyl 1′,2′-dihydrospiro[piperidine-4,3′-pyrrolo[3,2-b]pyridine]-1-carboxylate (9 g, 7.68 mmol, 25% purity, 78.9% yield). Flash chromatography (chloroform/acetonitrile) of this crude product afforded benzyl 1′,2′-dihydrospiro[piperidine-4,3′-pyrrolo[3,2-b]pyridine]-1-carboxylate (3 g, 4.63 mmol, 50% purity, 52.6% yield), which was used in the next step without further purification.

### Step-5. Synthesis of benzyl 1′-[4-(difluoromethyl)benzenesulfonyl]-1′,2′-dihydrospiro[piperidine-4,3′-pyrrolo[3,2-b]pyridine]-1-carboxylate

4-(Difluoromethyl)benzene-1-sulfonyl chloride (0.768 g, 3.39 mmol) was added to a mixture of benzyl 1′,2′-dihydrospiro[piperidine-4,3′-pyrrolo[3,2-b]pyridine]-1-carboxylate (2 g, 3.09 mmol, 50% purity) and pyridine (0.488 g, 6.18 mmol) in dry dichloromethane (25 mL). The reaction mixture was stirred at room temperature overnight and evaporated. The residue was subjected to HPLC purification (deionized water/HPLC-grade acetonitrile) to afford benzyl 1′-[4-(difluoromethyl)benzenesulfonyl]-1′,2′-dihydrospiro[piperidine-4,3′-pyrrolo[3,2-b]pyridine]-1-carboxylate (I-134). Yield: 251 mg, 15%; Appearance: Beige solid; ¹H NMR (400 MHz, DMSO-d₆) δ 8.17 (dd, J=4.0, 2.5 Hz, 1H), 8.03 (d, J=8.1 Hz, 2H), 7.86-7.72 (m, 3H), 7.41-7.29 (m, 5H), 7.29-6.93 (m, 2H), 5.08 (s, 2H), 3.96 (d, J=1.7 Hz, 2H), 3.86 (d, J=13.6 Hz, 2H), 3.20-2.96 (m, 2H), 1.60 (t, J=12.0 Hz, 2H), 1.21 (d, J=13.5 Hz, 2H); HPLC purity: %; LCMS Calculated for C₂₆H₂₅F₂N₃O₄S: 513.56; Observed: 514[M+H]+.

### Example 45—Synthesis of benzyl 1′-[4-(difluoromethyl)benzenesulfonyl]-1′,2′-dihydrospiro[piperidine-4,3′-pyrrolo[2,3-c]pyridine]-1-carboxylate (I-135)

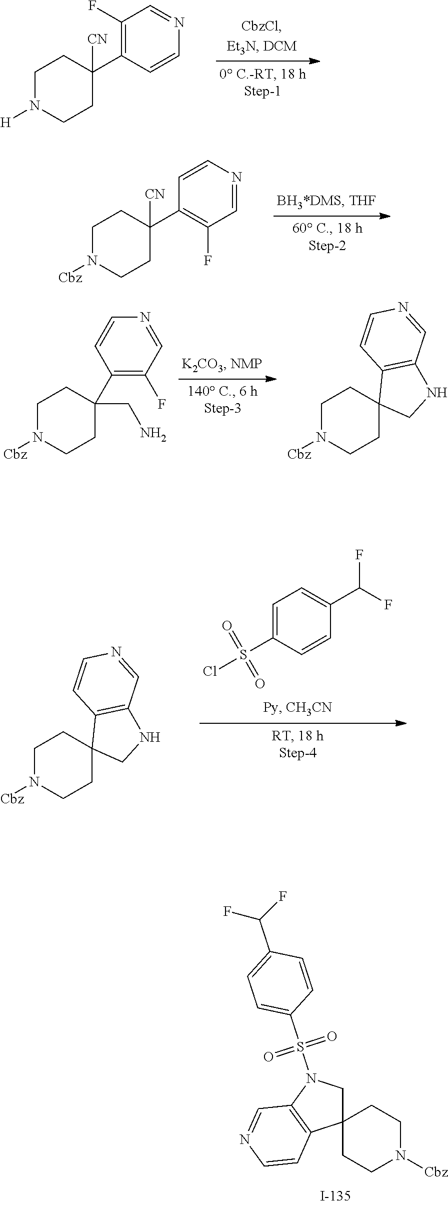

### Step-1. Synthesis of 1-benzyl 4-methyl piperidine-1,4-dicarboxylate

Triethylamine (1.74 mL, 12.6 mmol) and benzyl carbonochloridate (1.97 g, 11.6 mmol) were added to a solution of 4-(3-fluoropyridin-4-yl)piperidine-4-carbonitrile (2 g, 9.74 mmol) in dichloromethane (50 mL) at 0° C. The mixture was stirred at room temperature for 18 h until completion (TLC control). After, the reaction mixture was washed with water (50 mL) and brine (50 mL), dried over anhydrous sodium sulfate, and filtered through silica. The silica was washed with dichloromethane (250 mL), and the combined filtrates were evaporated under reduced pressure to give 1-benzyl 4-methyl piperidine-1,4-dicarboxylate as a colorless oil (2.7 g, 6.52 mmol, 82% purity, 66.9% yield) that was used in the next step without further purification.

### Step-2. Synthesis of benzyl 4-(aminomethyl)-4-(3-fluoropyridin-4-yl)piperidine-1-carboxylate

10 M (Methylsulfanyl)methane borane solution in tetrahydrofuran (3.23 mL, 32.4 mmol) was added to a solution of benzyl 4-cyano-4-(3-fluoropyridin-4-yl)piperidine-1-carboxylate (2.2 g, 5.30 mmol, 82% purity) in anhydrous tetrahydrofuran (150 mL) at room temperature. The reaction mixture was allowed to warm up and stir at 60° C. for 18 hours. Then, it was cooled to 15° C. and quenched with methanol (50 mL). The mixture was stirred for additional 1 h. After, it was concentrated under reduced pressure to afford benzyl 4-(aminomethyl)-4-(3-fluoropyridin-4-yl)piperidine-1-carboxylate as a colorless oil (2.5 g, 5.82 mmol, 80% purity, 90% yield) that was used in the next step without further purification.

### Step-3. Synthesis of benzyl 1′,2′-dihydrospiro[piperidine-4,3′-pyrrolo[2,3-c]pyridine]-1-carboxylate

Benzyl 4-(aminomethyl)-4-(3-fluoropyridin-4-yl)piperidine-1-carboxylate (2.5 g, 5.82 mmol, 80% purity) was added to a stirred solution of dipotassium carbonate (1.60 g, 11.6 mmol) in dry NMP (50 mL). The mixture was stirred at 140° C. for 6 hours. Then, it was cooled to room temperature, poured into water (100 mL), and extracted with ethyl acetate (50 mL×3). The organic layer was washed with water (100 mL) and brine (50 mL), dried over anhydrous sodium sulfate, filtered, and concentrated under reduced pressure. The residue was subjected to HPLC purification (deionized water/HPLC-grade acetonitrile) to give benzyl 1′,2′-dihydrospiro[piperidine-4,3′-pyrrolo[2,3-c]pyridine]-1-carboxylate as beige oil (0.0339 g, 89.1 μmol, 85% purity, 1.53% yield) that was used in the next step without further purification.

### Step-4. Synthesis of benzyl 1′-[4-(difluoromethyl)benzenesulfonyl]-1′,2′-dihydrospiro[piperidine-4,3′-pyrrolo[2,3-c]pyridine]-1-carboxylate

Pyridine (0.0123 g, 0.156 mmol) and 4-(difluoromethyl)benzene-1-sulfonyl chloride (0.0281 g, 0.124 mmol) were added to a solution of benzyl 1′,2′-dihydrospiro[piperidine-4,3′-pyrrolo[2,3-c]pyridine]-1-carboxylate (0.0339 g, 0.104 mmol) in acetonitrile (10 mL). The reaction mixture was stirred at room temperature for 18 h. The solvent was removed under reduced pressure and the residue was subjected to HPLC purification (deionized water/HPLC-grade acetonitrile) to afford benzyl 1′-[4-(difluoromethyl)benzenesulfonyl]-1′,2′-dihydrospiro[piperidine-4,3′-pyrrolo[2,3-c]pyridine]-1-carboxylate, (difluoromethyl)benzenesulfonyl]-1′,2′-dihydrospiro[piperidine-4,3′-pyrrolo[2,3-c]pyridine]-1-carboxylate (I-135). Yield: 19 mg, 4%; Appearance: Yellow solid; ¹H NMR (400 MHz, Chloroform-d) δ 8.93 (s, 1H), 8.35 (d, J=4.9 Hz, 1H), 7.97 (d, J=8.1 Hz, 2H), 7.66 (d, J=8.1 Hz, 2H), 7.42-7.31 (m, 5H), 7.01 (d, J=4.9 Hz, 1H), 6.83-6.49 (m, 1H), 5.16 (s, 2H), 4.27-4.00 (m, 2H), 3.82 (s, 2H), 2.98-2.79 (m, 2H), 1.79-1.58 (m, 3H), 1.36 (d, J=13.6 Hz, 2H); HPLC purity: 100%; LCMS Calculated for C₂₆H₂₅F₂N₃O₄S: 513.56; Observed: 514.2[M+H]⁺.

### Example 46—Synthesis of (1-((4-(difluoromethyl)phenyl)sulfonyl)spiro[indoline-3,4′-piperidin]-1′-yl)(pyrrolidin-1-yl)methanone (I-136)

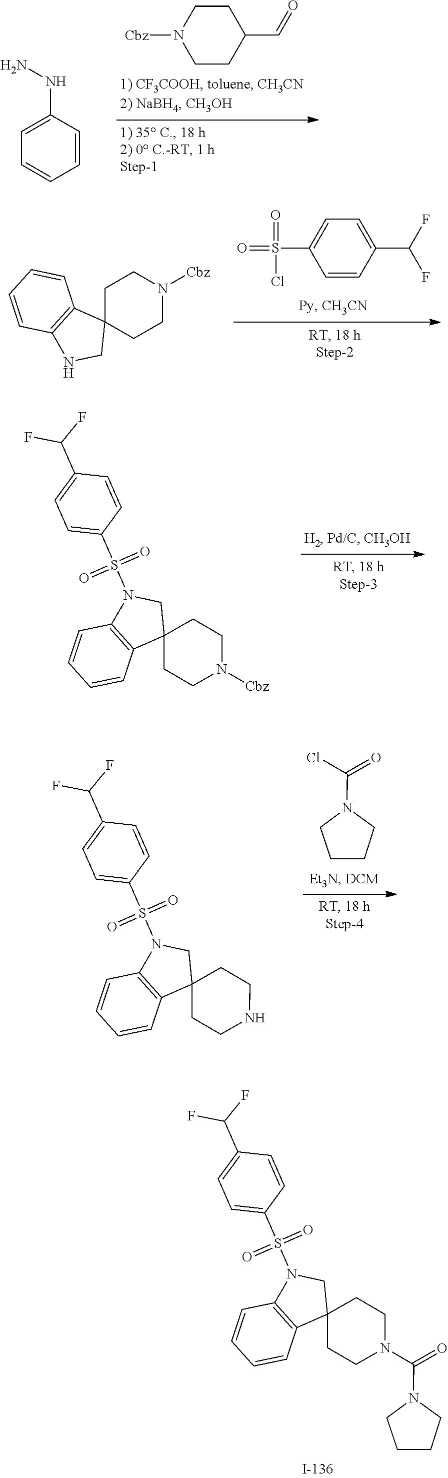

### Step-1. Synthesis of benzyl 1,2-dihydrospiro[indole-3,4′-piperidine]-1′-carboxylate

A solution of benzyl 4-formylpiperidine-1-carboxylate (27 g, 109 mmol) in toluene/acetonitrile (49/1, 100 mL) was added dropwise to a mixture of phenyl hydrazine (12.9 g, 102 mmol) and trifluoroacetic acid (30 mL) in a toluene/acetonitrile (49/1, 500 mL) at 35° C. The reaction mixture was stirred at 35° C. overnight. Then, the reaction mixture was cooled to 0° C. and diluted with methanol (50 mL). Sodium boranuide (6.19 g, 163 mmol) was added slowly to the reaction mixture. The mixture was stirred for 45 min, then washed with 6% NH₄OH aq. solution (250 mL) and brine (300 mL), dried over sodium sulfate, filtered, and evaporated. The residue was purified by flash chromatography (chloroform/MTBE) to afford benzyl 1,2-dihydrospiro[indole-3,4′-piperidine]-1′-carboxylate as white solid (22 g, 64.8 mmol, 95% purity, 63.7% yield).

### Step-2. Synthesis of benzyl 1-[4-(difluoromethyl)benzenesulfonyl]-1,2-dihydrospiro[indole-3,4′-piperidine]-1′-carboxylate

Pyridine (2.57 g, 32.5 mmol) and 4-(difluoromethyl)benzene-1-sulfonyl chloride (5.89 g, 26.0 mmol) were added to a solution of benzyl 1,2-dihydrospiro[indole-3,4′-piperidine]-1′-carboxylate (7 g, 21.7 mmol) in acetonitrile (200 mL). The reaction mixture was stirred at room temperature for 18 h. The solvent was evaporated under reduced pressure. Flash chromatography (hexane/chloroform/MTBE) purification of the residue afforded benzyl 1-[4-(difluoromethyl)benzenesulfonyl]-1,2-dihydrospiro[indole-3,4′-piperidine]-1′-carboxylate as beige solid (10.5 g, 20.4 mmol, 100% purity, 94.5% yield).

### Step-3. Synthesis of 1-[4-(difluoromethyl)benzenesulfonyl]-1,2-dihydrospiro[indole-3,4′-piperidine]

Benzyl 1-[4-(difluoromethyl)benzenesulfonyl]-1,2-dihydrospiro[indole-3,4 (7.5 g, 14.6 mmol) was dissolved in a MeOH/tetrahydrofuran mixture (1/1, 300 mL). Pd/C (20%, 3 g) was added to the solution, and then the black suspension was hydrogenated at ambient pressure and room temperature for 18 h. Then, the mixture was filtered, the precipitate was washed with tetrahydrofuran (200 mL), and the combined filtrates were concentrated under reduced pressure to afford 1-[4-(difluoromethyl)benzenesulfonyl]-1,2-dihydrospiro[indole-3,4′-piperidine] as beige oil (3.65 g, 9.25 mmol, 96% purity, 63.4% yield).

### Step-4. Synthesis of (1-((4-(difluoromethyl)phenyl)sulfonyl)spiro[indoline-3,4′-piperidin]-1′-yl)(pyrrolidin-1-yl)methanone]

Triethylamine (0.217 μL, 1.57 mmol) and pyrrolidine-1-carbonyl chloride (0.168 g, 1.26 mmol) were added to a solution of 1-[4-(difluoromethyl)benzenesulfonyl]-1,2-dihydrospiro[indole-3,4 (0.4 g, 1.05 mmol) in dichloromethane (50 mL). The reaction mixture was stirred at room temperature for 18 h. The solvent was removed under reduced pressure, and the residue was subjected to HPLC purification (deionized water/HPLC-grade acetonitrile) to afford (1-((4-(difluoromethyl)phenyl)sulfonyl)spiro[indoline-3,4′-piperidin]-1′-yl)(pyrrolidin-1-yl)methanone] (I-136). Yield: 166 mg, 31.4%; Appearance: White solid; ¹H NMR (400 MHz, DMSO-d₆) δ 8.00 (d, J=8.1 Hz, 2H), 7.78 (d, J=8.1 Hz, 2H), 7.51 (d, J=8.1 Hz, 1H), 7.28-6.95 (m, 4H), 3.92 (s, 2H), 3.52 (d, J=13.4 Hz, 2H), 3.25 (d, J=13.0 Hz, 4H), 2.78 (t, J=12.9 Hz, 2H), 1.79-1.68 (m, 4H), 1.61 (td, J=12.9, 4.0 Hz, 2H), 1.12-1.02 (m, 2H); HPLC purity: 100%; LCMS Calculated for C₂₄H₂₇F₂N₃O₃S: 475.55; Observed: 476.2[M+H]⁺.

### Example 47—Synthesis of (1r,4r)-1′-[4-(difluoromethyl)benzenesulfonyl]-4-(ethoxymethyl)-4-methyl-1,2′-dihydrospiro[cyclohexane-1,3′-indole] (I-137)

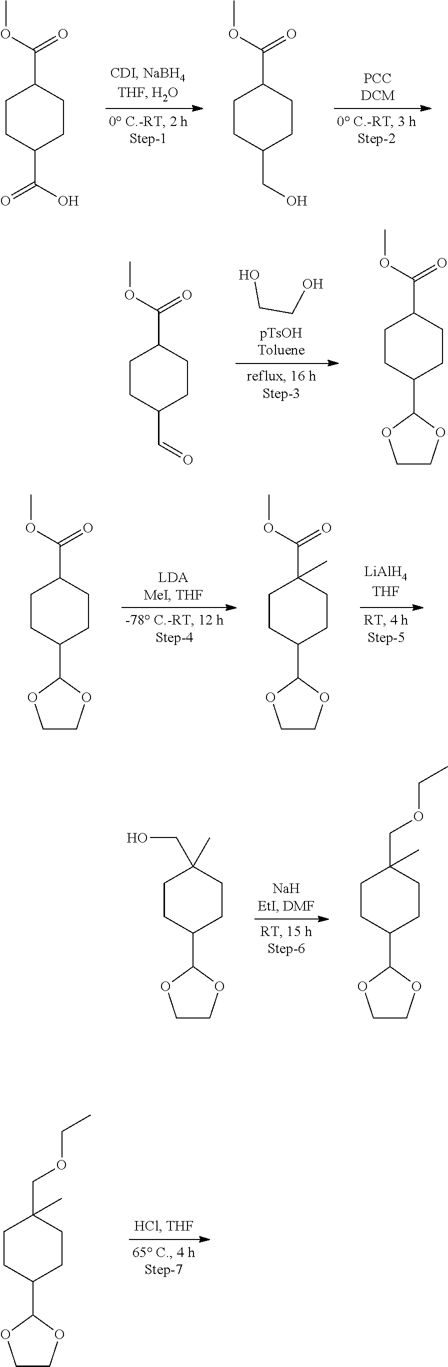

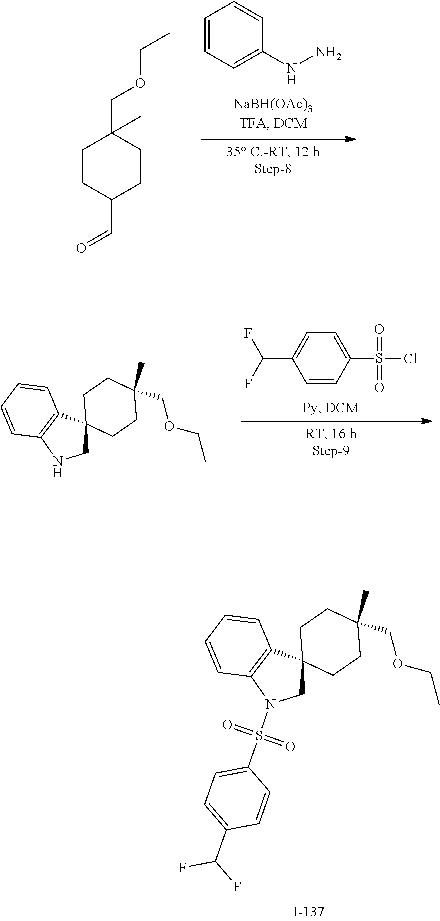

### Step-1. Synthesis of methyl 4-(hydroxymethyl)cyclohexane-1-carboxylate

1-(1H-imidazole-1-carbonyl)-1H-imidazole (5.20 g, 32.1 mmol) was added at 0° C. to a stirred solution of 4-(methoxycarbonyl)cyclohexane-1-carboxylic acid (5 g, 26.8 mmol) in tetrahydrofuran (50 mL). The reaction mixture was stirred for 1 h at 0° C., and then sodium borohydride (1.51 g, 40.1 mmol) was added. The reaction mixture was stirred at room temperature for 1 h. The progress of the reaction was monitored by TLC. After completion, the reaction mixture was diluted with water (50 mL) and extracted with ethyl acetate (50 mL×2). The combined organic extracts were washed with citric acid (50 mL), sodium bicarbonate sat. aq. solution (50 mL), and brine (50 mL), dried over anhydrous sodium sulfate, filtered, and concentrated under reduced pressure to provide methyl 4-(hydroxymethyl)cyclohexane-1-carboxylate (3.5 g, 12.2 mmol, 90% purity, 68% yield) that was used in the next step without further purification.

### Step-2. Synthesis of methyl 4-formylcyclohexane-1-carboxylate

Methyl 4-(hydroxymethyl)cyclohexane-1-carboxylate (3.3 g, 19.1 mmol) was dissolved in dichloromethane (30 mL). The solution was cooled to 0° C., and chlorochromiumoylol; pyridine (5.34 g, 24.8 mmol) was added. The reaction mixture was warmed to room temperature, stirred for 3 h until completion (TLC control), and filtered through a Celite pad. The filtrate was evaporated under reduced pressure. The crude residue was dissolved in diethyl ether (100 mL) and filtered through Celite. The filtrate was evaporated to provide methyl 4-formylcyclohexane-1-carboxylate (2.4 g, 12.6 mmol, 90°/% purity, 66.4% yield) that was used in the next step without further purification.

### Step-3. Synthesis of methyl 4-(1,3-dioxolan-2-yl)cyclohexane-1-carboxylate

A solution of 4-(methoxycarbonyl)-cyclohexanecarboxaldehyde (4 g, 23.5 mmol), 1,2-ethanediol (2.91 g, 47.0 mmol), and p-toluenesulfonic acid monohydrate (0.201 g, 1.17 mmol) in benzene (100 mL) was refluxed under a Dean-Stark trap overnight. The mixture was cooled to room temperature, diluted with ether (100 mL), and washed with water (100 mL×3), followed by washing with NaHCO₃ sat. aq. solution (100 mL). The organic phase was dried over magnesium sulfate, filtered, and concentrated to give methyl 4-(1,3-dioxolan-2-yl)cyclohexane-1-carboxylate (3.8 g, 14.1 mmol, 80% purity, 60.4% yield) that was used in the next step without further purification.

### Step-4. Synthesis of methyl 4-(1,3-dioxolan-2-yl)-1-methylcyclohexane-1-carboxylate

A 2.5M n-BuLi solution in hexane (2.7 mL, 6.71 mmol) was added at 0° C. to a solution of bis(propan-2-yl)amine (1.02 mL, 7.28 mmol) in 20 mL of anhydrous tetrahydrofuran. The mixture was stirred at 0° C. for 20 min. A solution of 4-(1,3-dioxolan-2-yl)cyclohexane-1-carboxylate (1.2 g, 5.60 mmol) in tetrahydrofuran (10 mL) was added dropwise after. The mixture was then stirred at 0° C. for 1 h. Then, the mixture was cooled to −78° C., and iodomethane (1.58 g, 1.2 mmol) was added dropwise. After the addition was completed, the mixture was stirred at −78° C. for an additional 1 h, then warmed to 15° C. and stirred for 12 h. The resulting mixture was poured into saturated aq. NH₄Cl solution (50 mL) and extracted with ethyl acetate (30 mL×3). The combined organic layers were washed with brine (100 mL×2), dried over anhydrous sodium sulfate, filtered, and concentrated in vacuo to afford methyl 4-(1,3-dioxolan-2-yl)-1-methylcyclohexane-1-carboxylate (1.2 g, 4.20 mmol, 80% purity, 75.5% yield) that was used in the next step without further purification.

### Step-5. Synthesis of [4-(1,3-dioxolan-2-yl)-1-methylcyclohexyl]methanol

Methyl 4-(1,3-dioxolan-2-yl)-1-methylcyclohexane-1-carboxylate (1.2 g, 5.25 mmol) solution in tetrahydrofuran (3 mL) was added to a solution of LiAlH₄ (0.196 g, 5.8 mmol) in tetrahydrofuran (10 mL). The reaction mixture was stirred at room temperature for 4 h. Then, it was quenched with water (0.8 mL) and 6N NaOH aq. solution (0.2 mL). The mixture was filtered, and the filter cake was washed with tetrahydrofuran (10 mL×2). The combined filtrates were evaporated under reduced pressure to afford [4-(1,3-dioxolan-2-yl)-1-methylcyclohexyl]methanol (1.05 g, 4.71 mmol, 90% purity, 90% yield) that was used in the next step without further purification.

### Step-6. Synthesis of 2-[4-(ethoxymethyl)-4-methylcyclohexyl]-1,3-dioxolane

Sodium hydride (0.230 g, 5.76 mmol, 60 w % dispersion in mineral oil) was added to an ice-cooled solution of [4-(1,3-dioxolan-2-yl)-1-methylcyclohexyl]methanol (1.05 g, 5.24 mmol) in tetrahydrofuran (15 mL). The reaction mixture was warmed to room temperature. After 3 h, iodoethane (1.22 g, 7.86 mmol) was added, and the reaction mixture was stirred at room temperature for 12 h. The excess sodium hydride was quenched with methanol (5 mL), and the reaction mixture was concentrated under reduced pressure to give 2-[4-(ethoxymethyl)-4-methylcyclohexyl]-1,3-dioxolane (1.2 g, 4.20 mmol, 80% purity, 80.6% yield) that was used in the next step without further purification.

### Step-7. Synthesis of 4-(ethoxymethyl)-4-methylcyclohexane-1-carbaldehyde

A solution of 2-[4-(ethoxymethyl)-4-methylcyclohexyl]-1,3-dioxolane (1.2 g, 4.2 mmol) and 1N aq. HCl (14 mL) in tetrahydrofuran (45 mL) was heated at 65° C. for 4 h. The reaction mixture was cooled to room temperature, diluted with water (40 mL), and extracted with ethyl acetate (80 mL×2). The combined organic extracts were washed with water (30 mL) and brine (30 mL), dried over anhydrous sodium sulfate, filtered, and concentrated under reduced pressure to provide 4-(ethoxymethyl)-4-methylcyclohexane-1-carbaldehyde (0.8 g, 3.03 mmol, 70% purity, 57.9% yield) that was used in the next step without further purification.

### Step-8. Synthesis of 4-(ethoxymethyl)-4-methyl-1′,2′-dihydrospiro[cyclohexane-1,3′-indole]

A solution of 4-(ethoxymethyl)-4-methylcyclohexane-1-carbaldehyde (0.8 g, 4.34 mmol) in dichloromethane (5 mL) was added dropwise to a mixture of phenyl hydrazine (0.469 g, 4.34 mmol) and trifluoroacetic acid (4.94 g, 43.4 mmol) in dichloromethane (10 mL). The mixture was stirred at 35° C. overnight. The mixture was then cooled to 0° C., and sodium triacetoxyborohydride (2.75 g, 13.0 mmol) was added slowly, followed by stirring at room temperature for 4 hours. The mixture was then washed with 6% NH₄OH aq. solution (25 mL) and brine (30 mL), dried over sodium sulfate, filtered, and evaporated to dryness. The crude residue was purified by flash chromatography (hexane/MTBE) to provide 4-(ethoxymethyl)-4-methyl-1′,2′-dihydrospiro[cyclohexane-1,3′-indole] (0.17 g, 0.589 mmol, 90% purity, 13.6% yield).

### Step-9. Synthesis of (1r,4r)-1′-[4-(difluoromethyl)benzenesulfonyl]-4-(ethoxymethyl)-4-methyl-1′,2′-dihydrospiro[cyclohexane-1,3′-indole]

4-(Difluoromethyl)benzene-1-sulfonyl chloride (0.178 g, 0.786 mmol) was added to an ice-cooled solution of 4-(ethoxymethyl)-4-methyl-1′,2′-dihydrospiro[cyclohexane-1,3′-indole](0.17 g, 0.655 mmol) and pyridine (0.258 g, 3.27 mmol) in dichloromethane (10 mL). The reaction mixture was allowed to warm to room temperature and stir until completion (overnight, NMR control). The reaction mixture was then diluted with water (10 mL), and the organic layer was separated, dried over magnesium sulfate, filtered, and concentrated in vacuo. The residue was subjected to HPLC purification (deionized water/HPLC-grade acetonitrile) to give (1r,4r)-1′-[4-(difluoromethyl)benzenesulfonyl]-4-(ethoxymethyl)-4-methyl-1′,2′-dihydrospiro[cyclohexane-1,3′-indole] (I-137). Yield: 95.6 mg, 30.8%; Appearance: Pink solid; ¹H NMR (600 MHz, DMSO-d₆) δ 7.96 (d, J=8.1 Hz, 2H), 7.74 (d, J=8.1 Hz, 2H), 7.48 (d, J=8.0 Hz, 1H), 7.27-7.18 (m, 2H), 7.17-6.97 (m, 2H), 3.78 (s, 2H), 3.41 (q, J=7.0 Hz, 2H), 3.25 (s, 2H), 1.58-1.48 (m, 2H), 1.46-1.38 (m, 2H), 1.19-1.11 (m, 2H), 1.08 (t, J=7.0 Hz, 3H), 0.91 (d, J=13.5 Hz, 2H), 0.87 (s, 3H), HPLC purity: 97.86%; LCMS Calculated for C₂₄H₂₉F₂NO₃S: 449.55; Observed: 450.2[M+H]⁺.

### Example 48—Synthesis of 1′-[4-(difluoromethyl)benzenesulfonyl]-4,4-difluoro-1′,2′-dihydrospiro[cyclohexane-1,3′-indole] (I-138)

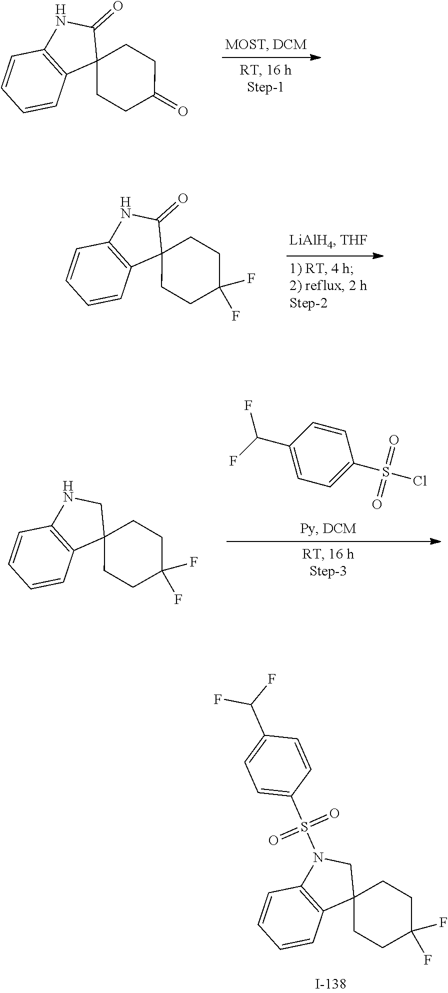 [SMILES: O=C1CCC2(CC1)C(=O)Nc1ccccc12 ; O=C1Nc2ccccc2C12CCC(F)(F)CC2 ; FC1(F)CCC2(CC1)CNc1ccccc12 ; O=S(=O)(Cl)c1ccc(C(F)F)cc1 ; O=S(=O)(c1ccc(C(F)F)cc1)N1CC2(CCC(F)(F)CC2)c2ccccc21]

### Step-1. Synthesis of 4,4-difluoro-1′,2′-dihydrospiro[cyclohexane-1,3′-indol]-2′-one

Morpholinosulfur trifluoride (12.1 g, 69.6 mmol)) was added to a stirred solution of 1′,2′-dihydrospiro[cyclohexane-1,3′-indole]-2′,4-dione (5 g, 23.2 mmol) in dichloromethane (50 mL). The resulting mixture was stirred overnight and evaporated to dryness. The crude residue was treated with sat. sodium carbonate aq. Solution, and the mixture was extracted with dichloromethane (30 mL×2). The organic layers were combined, dried over anhydrous sodium sulfate, filtered, and concentrated under reduced pressure. The crude residue was purified by flash chromatography (hexane/ethyl acetate) to provide 4,4-difluoro-1′,2′-dihydrospiro[cyclohexane-1,3′-indol]-2′-one (0.92 g, 3.87 mmol, 95% purity, 15.8% yield).

### Step-2. Synthesis of 4,4-difluoro-1′,2′-dihydrospiro[cyclohexane-1,3′-indole]

A solution of 4,4-difluoro-1′,2′-dihydrospiro[cyclohexane-1,3′-indol]-2′-one (0.92 g, 3.87 mmol) in tetrahydrofuran (3 mL) was added to a solution of LiAlH₄ (0.196 g, 5.8 mmol) in tetrahydrofuran (10 mL). The reaction mixture was stirred at room temperature for 4 h. It was then refluxed for 2 h, cooled to room temperature, and quenched with water (0.8 mL) and 6N NaOH aq. solution (0.2 mL). The mixture was filtered, and the filter cake was washed with tetrahydrofuran (10 mL×2). The combined filtrates were evaporated under reduced pressure to afford 4,4-difluoro-1′,2′-dihydrospiro[cyclohexane-1,3′-indole] (0.8 g, 3.58 mmol, 95% purity, 87.9% yield).

### Step-3. Synthesis of 1′-[4-(difluoromethyl)benzenesulfonyl]-4,4-difluoro-1′,2′-dihydrospiro[cyclohexane-1,3′-indole]

4-(difluoromethyl)benzene-1-sulfonyl chloride (0.222 g, 0.984 mmol) was added to an ice-cooled solution of 4,4-difluoro-1′,2′-dihydrospiro[cyclohexane-1,3′-indole] (0.2 g, 0.895 mmol) and pyridine (0.353 g, 4.47 mmol) in dichloromethane (10 mL). The reaction mixture was allowed to warm to room temperature and stir until completion (overnight, NMR control). Afterwards, the reaction mixture was diluted with water (10 mL), and the organic layer was separated, dried over magnesium sulfate, filtered, and concentrated in vacuo. The residue was subjected to HPLC purification (deionized water/HPLC-grade methanol) to give 1′-[4-(difluoromethyl)benzenesulfonyl]-4,4-difluoro-1′,2′-dihydrospiro[cyclohexane-1,3′-indole] (I-138). Yield: 22.7 mg, 5.81%; Appearance: Pink solid; ¹H NMR (600 MHz, DMSO-d) S 8.00 (d, J=8.1 Hz, 2H), 7.75 (d, J=8.1 Hz, 2H), 7.50 (d, J=8.1 Hz, 1H), 7.28-7.22 (m, 1H), 7.18 (d, J=7.4 Hz, 1H), 7.04 (dd, J=31.4, 24.0 Hz, 2H), 3.94 (s, 2H), 2.00 (t, J=14.6 Hz, 1H), 1.96-1.83 (m, 3H), 1.66 (td, J=13.6, 4.0 Hz, 2H), 1.18 (d, J=13.6 Hz, 2H); HPLC purity. 100%; LCMS Calculated for C₂₀H₁₉F₄NO₂S: 413.3; Observed: 414.0[M+H]⁺.

### Example 49—Synthesis of benzyl 1-((4-(cyclopropyldifluoromethyl)phenyl)sulfonyl)spiro[indoline-3,4′-piperidine]-1′-carboxylate (I-139)

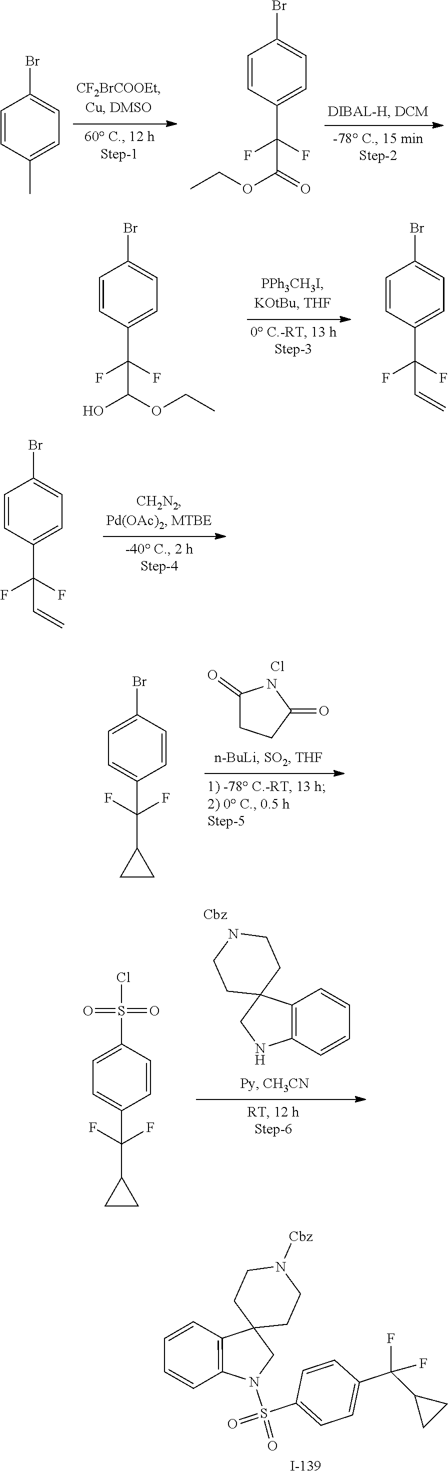

### Step-1. Synthesis of ethyl 2-(4-bromophenyl)-2,2-difluoroacetate

1-bromo-4-iodobenzene (20 g, 70.6 mmol) and ethyl 2-bromo-2,2-difluoroacetate (15.7 g, 77.6 mmol) were added under argon atmosphere to a suspension of activated copper powder (11.6 g, 183 mmol) in DMSO (200 mL), and the mixture was stirred at 60° C. for 12 h. Afterwards, the mixture was poured into a mixture of ice (400 g) and NH₄Cl sat. aq. solution (200 mL), and the product was extracted with MTBE (200 mL×3). The combined MTBE layers were washed with NH₄Cl saturated aq. solution (400 mL) and brine (400 mL), dried over sodium sulfate, filtered, and evaporated under reduced pressure. The residue was purified by column chromatography (hexane/chloroform) to afford ethyl 2-(4-bromophenyl)-2,2-difluoroacetate as a light-yellow oil (14.2 g, 48.3 mmol, 95% purity, 68% yield).

### Step-2. Synthesis of 2-(4-bromophenyl)-1-ethoxy-2,2-difluoroethanol

A 1 M solution of DIBAL (8.66 g, 60.9 mmol) in cyclohexane (60.9 mL) was added dropwise at −78° C. under argon atmosphere to a solution of ethyl 2-(4-bromophenyl)-2,2-difluoroacetate (14.2 g, 50.8 mmol) in dry dichloromethane (150 mL). The reaction mixture was stirred at −78° C. for 15 min, then poured into 10% HCl aq. solution (150 mL). The mixture was extracted with dichloromethane (150 mL×2). The combined organic layers were washed with brine (150 mL), dried over sodium sulfate, filtered, and evaporated under reduced pressure to obtain 2-(4-bromophenyl)-1-ethoxy-2,2-difluoroethanol as a white solid (11.9 g, 42.3 mmol, 97% purity, 80.9% yield).

### Step-3. Synthesis of 1-bromo-4-(1,1-difluoroallyl)benzene

Methyltriphenylphosphoniumbromide (59.8 g, 148 mmol) was suspended in dry tetrahydrofuran (200 mL) under argon atmosphere, and (tert-butoxy)potassium (16.6 g, 148 mmol) was added at 0° C. over 30 min. The mixture was stirred at 0° C. for 1 h. Then, 2-(4-bromophenyl)-1-ethoxy-2,2-difluoroethanol (11.9 g, 42.3 mmol) was added, and the mixture was stirred at room temperature for 12 h. Afterwards, the reaction was diluted with water (200 mL). The product was extracted with MTBE (200 mL×2). The combined organic layers were washed with brine (200 mL), dried over sodium sulfate, filtered, and evaporated in vacuo. The crude product was purified by flash chromatography (hexane/chloroform) to give 1-bromo-4-(1,1-difluoroallyl)benzene as a colorless oil (6.85 g, 27.9 mmol, 95% purity, 65.9% yield).

### Step-4. Synthesis of 1-bromo-4-(cyclopropyldifluoromethyl)benzene

A 0.8 M solution of diazomethane (6.13 g, 146 mmol) in MTBE (182 mL) was added at −40° C. to a mixture of 1-bromo-4-(1,1-difluoroallyl)benzene (6.85 g, 29.3 mmol) and Pd(OAc)₂ (0.327 g, 1.46 mmol) in dry MTBE (200 mL). The mixture was stirred at −40° C. until the evolution of gas ceased (for 2 h). The mixture was then filtered, and the filtrate was concentrated in vacuo. The residue was purified by flash chromatography (hexane/MTBE) to give 1-bromo-4-(cyclopropyldifluoromethyl)benzene as a colorless oil (3.5 g, 13.6 mmol, 96.39% purity, 46.6% yield).

### Step-5. Synthesis of -(cyclopropyldifluoromethyl)benzene-1-sulfonyl chloride

2 M n-Butyllithium (384 mg, 6 mmol) in hexane (2.4 mL) was added to a solution of 1-bromo-4-(cyclopropyldifluoromethyl)benzene (1.23 g, 5 mmol) in anhydrous tetrahydrofuran (20 mL) at −78° C. under argon atmosphere, and the mixture was stirred for 1 h at this temperature. A solution of SO₂ (0.96 g, 15 mmol) in tetrahydrofuran (10 mL) was added to the resulting mixture at −78° C. The mixture was allowed to warm to room temperature and stirred for 12 h. The solution was evaporated in vacuo. The residue was dissolved in dichloromethane (20 mL) and N-chlorosuccinimide (0.801 g, 6 mmol) was added portionwise, maintaining the mixture temperature at 0° C. The mixture was stirred for 30 minutes, diluted with water (50 mL), and extracted with ethyl acetate (50 mL×2). The organic layer was dried over sodium sulfate and filtered, and the filtrate was evaporated in vacuo. The residue was purified by flash chromatography (hexane/CHCl₃) to give 4-(cyclopropyldifluoromethyl)benzene-1-sulfonyl chloride as a colorless oil (0.18 g, 0.674 mmol, 91% purity, 12.2% yield).

### Step-6. Synthesis of benzyl 1-((4-(cyclopropyldifluoromethyl)phenyl)sulfonyl)spiro[indoline-3,4′-piperidine]-1′-carboxylate

4-(Cyclopropyldifluoromethyl)benzene-1-sulfonyl chloride (0.18 g, 0.674 mmol) was added to a mixture of benzyl spiro[indoline-3,4′-piperidine]-1′-carboxylate (0.217 g, 0.674 mmol) and pyridine (0.266 g, 3.37 mmol) in dry acetonitrile (20 mL). The reaction mixture was stirred for 12 h and evaporated in vacuo. The residue was subjected to HPLC purification (deionized water/HPLC-grade acetonitrile) to afford benzyl 1-((4-(cyclopropyldifluoromethyl)phenyl)sulfonyl)spiro[indoline-3,4′-piperidine]-1′-carboxylate (I-139). Yield: 45.1 mg, 11.5%; Appearance: Pink solid; ¹H NMR (400 MHz, DMSO-d) S 7.94 (d, J=8.1 Hz, 2H), 7.76 (d, J=8.1 Hz, 2H), 7.51 (d, J=8.1 Hz, 1H), 7.36 (s, 6H), 7.31-7.19 (m, 3H), 7.05 (t, J=7.3 Hz, 1H), 5.07 (s, 2H), 3.93 (s, 2H), 3.86 (d, J=14.3 Hz, 2H), 2.95 (s, 2H), 1.67 (s, 1H), 1.56 (s, 2H), 1.03 (s, 2H), 0.63 (d, J=11.6 Hz, 4H); HPLC purity: 100%; LCMS Calculated for C₃₀H₃₀F₂N₂OS: 552.64; Observed: 554.2 [M+H]⁺.

### Example 50—Synthesis of benzyl 1′-[4-(difluoromethyl)benzenesulfonyl]-1′,2′-dihydrospiro[piperidine-4,3-pyrrolo[3,2-b]pyridine]-1-carboxylate (I-140)

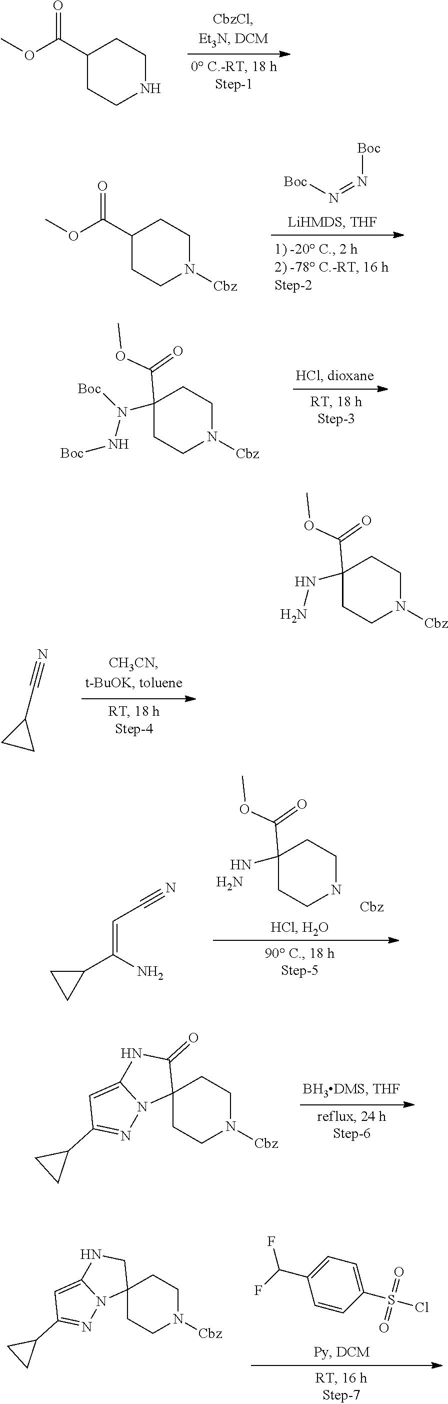

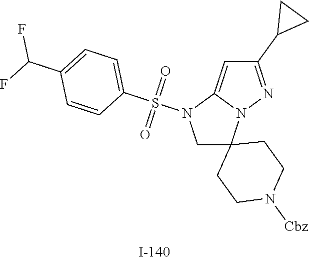 [SMILES: O=C(OCc1ccccc1)N1CCC2(CC1)CN(S(=O)(=O)c1ccc(C(F)F)cc1)c1cc(C3CC3)nn12]

### Step-1. Synthesis of 1-benzyl 4-methyl piperidine-1,4-dicarboxylate

Triethylamine (15.7 g, 156 mmol) and benzyl carbonochloridate (23.0 g, 135 mmol) were added to a solution of methyl piperidine-4-carboxylate (15 g, 104 mmol) in dichloromethane (500 mL) at 0° C. The mixture was stirred at room temperature for 18 h until completion (TLC control). Afterwards, the reaction mixture was washed with water (500 mL) and brine (500 mL), dried over anhydrous sodium sulfate, and filtered through silica. The silica was washed with dichloromethane (250 mL), and the combined filtrates were evaporated under reduced pressure. The residue was purified by flash chromatography (hexane/MTBE) that afforded 1-benzyl 4-methyl piperidine-1,4-dicarboxylate as colorless oil (28.8 g, 103 mmol, 95% purity, 91.3% yield).

### Step-2. Synthesis of 1-benzyl 4-methyl 4-{[(tert-butoxy)carbonyl]({[(tert-butoxy)carbonyl]-amino})amino}piperidine-1,4-dicarboxylate

A 1 M solution of lithium bis(trimethylsilyl)amide in tetrahydrofuran (21.6 mL, 21.6 mmol) was added dropwise at −30° C. to a stirred solution of 1-benzyl 4-methyl piperidine-1,4-dicarboxylate (5 g, 18.0 mmol) in dry tetrahydrofuran (100 mL) under argon atmosphere, and the reaction mixture was stirred at −20° C. for 2 h. Then, a solution of (E)-N-{[(tert-butoxy)carbonyl]imino}(tert-butoxy)formamide (5.38 g, 23.4 mmol) in dry tetrahydrofuran (50 mL) was added at −78° C. Afterward, the reaction mixture was allowed to warm up and stir overnight at room temperature. Then, it was poured in water (250 mL) and extracted with ethyl acetate (100 mL×3). The organic layer was washed with water (100 mL) and brine (100 mL), dried over anhydrous sodium sulfate, filtered, and concentrated under reduced pressure to give 1-benzyl 4-methyl 4-({[(tert-butoxy)carbonyl](([(tert-butoxy)carbonyl]amino))amino}piperidine-1,4-dicarboxylate as a beige oil (10.2 g, 13.6 mmol, 68% purity, 76.9% yield) that was used in the next step without further purification.

### Step-3. Synthesis of 1-benzyl 4-methyl 4-hydrazinylpiperidine-1,4-dicarboxylate dihydrochloride

1-Benzyl 4-methyl 4-{[(tert-butoxy)carbonyl](([(tert-butoxy)carbonyl]amino))amino}piperidine-1,4-dicarboxylate (10.2 g, 13.6 mmol, 68% purity) was added to a stirred HCl aq. sat. solution in dry dioxane (100 mL). The reaction mixture was stirred at room temperature for 18 h. The residue was concentrated under reduced pressure to afford 1-benzyl 4-methyl 4-hydrazinylpiperidine-1,4-dicarboxylate dihydrochloride as a beige oil (7.3 g, 5.75 mmol, 30% purity, 42.3% yield) that was used in the next step without further purification.

### Step-4. Synthesis of (2Z)-3-amino-3-cyclopropylprop-2-enenitrile

(tert-Butoxy)potassium (25 g, 223 mmol) was added to a stirred solution of cyclopropanecarbonitrile (10 g, 149 mmol) and acetonitrile (9.15 g, 223 mmol) in dry toluene (300 mL). The mixture was stirred at room temperature for 18 h. Afterward, the reaction mixture was poured into water (500 mL) and extracted with ethyl acetate (250 mL×3). The organic layer was washed with water (500 mL) and brine (500 mL), dried over anhydrous sodium sulfate, filtered, and concentrated under reduced pressure to give (2Z)-3-amino-3-cyclopropylprop-2-enenitrile as white oil (4.5 g, 20.8 mmol, 50% purity, 13.9% yield) that was used in the next step without further purification.

### Step-5. Synthesis of benzyl 6′-cyclopropyl-2′-oxo-1′,2′-dihydrospiro[piperidine-4,3′-pyrazolo[1,5-a]imidazole]-1-carboxylate

1-Benzyl 4-methyl 4-hydrazinylpiperidine-1,4-dicarboxylate dihydrochloride (7.3 g, 5.75 mmol. 30% purity) and (2Z)-3-amino-3-cyclopropylprop-2-enenitrile (1.86 g, 8.62 mmol, 30% purity) were added to a 10% HCl aq. solution (100 mL). The reaction mixture was stirred at 90° C. for 18 h, cooled to room temperature, and concentrated under reduced pressure. The residue was poured into a 10% aq. solution of potassium carbonate (200 mL) and extracted with ethyl acetate (100 mL×3). The organic layer was washed with water (300 mL) and brine (300 mL), dried over anhydrous sodium sulfate, filtered, and concentrated under reduced pressure. The residue was subjected to flash chromatography (hexane/MTBE) purification to afford benzyl 6′-cyclopropyl-2′-oxo-1′,2′-dihydrospiro[piperidine-4,3′-pyrazolo[1,5-a]imidazole]-1-carboxylate as beige solid (0.2 g, 316 μmol, 53% purity, 5.04% yield) that was used in the next step without further purification.

### Step-6. Synthesis of benzyl 6′-cyclopropyl-1′,2′-dihydrospiro[piperidine-4,3′-pyrazolo[1,5-a]imidazole]-1-carboxylate

10 M Borane dimethyl sulfide complex in tetrahydrofuran (0.1 mL, 1.09 mmol) was added to a solution of benzyl 6′-cyclopropyl-2′-oxo-1′,2′-dihydrospiro[piperidine-4,3′-pyrazolo[1,5-a]imidazole]-1-carboxylate (0.2 g, 0.289 mmol, 53% purity) in tetrahydrofuran (10 mL), and the mixture was refluxed for 24 h. Afterwards, it was diluted with methanol (10 mL) and extracted with ethyl acetate (50 mL×2). The combined organic layers were washed with citric acid aq. sat. solution (50 mL), sodium hydrogen carbonate aq. sat. solution (50 mL), dried over sodium sulfate, filtered and evaporated under reduced pressure to give benzyl 6′-cyclopropyl-1′,2′-dihydrospiro[piperidine-4,3′-pyrazolo[1,5-a]imidazole]-1-carboxylate (0.13 g, 0.265 mmol, 72% purity, 92.6% yield) that was used in the next step without further purification.

### Step-7. Synthesis of benzyl 1′-[4-(difluoromethyl)benzenesulfonyl]-1′,2′-dihydrospiro[piperidine-4,3′-pyrrolo[3,2-b]pyridine]-1-carboxylate

4-(Difluoromethyl)benzene-1-sulfonyl chloride (0.06 g, 0.268 mmol) was added to a mixture of benzyl 6′-cyclopropyl-1′,2′-dihydrospiro[piperidine-4,3′-pyrazolo[1,5-a]imidazole]-1-carboxylate (0.13 g, 0.265 mmol, 72% purity) and pyridine (0.2 g, 2.68 mmol) in dry dichloromethane (25 mL). The reaction mixture was stirred at room temperature overnight and evaporated. The residue was subjected to HPLC purification (deionized water/HPLC-grade acetonitrile) to afford benzyl 6′-cyclopropyl-1′-[4-(difluoromethyl)benzenesulfonyl]-1′,2′-dihydrospiro[piperidine-4,3′-pyrazolo[1,5-a]imidazole]-1-carboxylate (I-140). Yield: 31.8 mg, 20.8%; Appearance: White solid; ¹H NMR (400 MHz, DMSO-d₆) δ 8.02 (d, J=8.1 Hz, 2H), 7.84 (d, J=8.1 Hz, 2H), 7.42-7.30 (m, 5H), 7.30-6.98 (m, 1H), 5.69 (s, 1H), 5.07 (s, 2H), 4.16 (s, 2H), 3.77-3.63 (m, 2H), 3.29-3.13 (m, 2H), 1.84-1.74 (m, 1H), 1.63-1.52 (m, 2H), 1.46-1.37 (m, 2H), 0.88-0.77 (m, 2H), 0.70-0.59 (m, 2H); HPLC purity: 100%; LCMS Calculated for C₂₇H₂₅F₂N₄O₄S: 542.6; Observed: 543.0[M+H]⁺.

### Example 51—Synthesis of pyridin-2-ylmethyl 1-((4-(difluoromethyl)phenyl)sulfonyl)spiro[indoline-3,4′-piperidine]-1′-carboxylate (I-141)

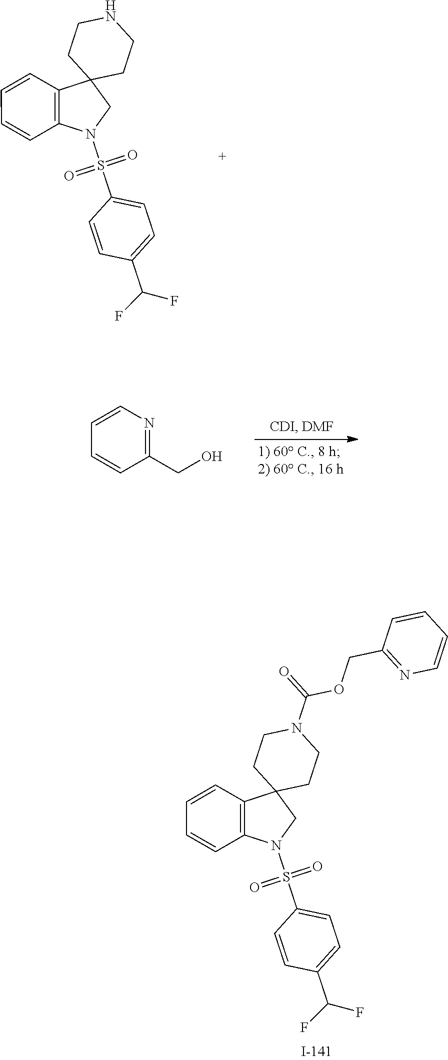 [SMILES: O=S(=O)(c1ccc(C(F)F)cc1)N1CC2(CCNCC2)c2ccccc21 ; OCc1ccccn1 ; O=C(OCc1ccccn1)N1CCC2(CC1)CN(S(=O)(=O)c1ccc(C(F)F)cc1)c1ccccc12]

(Pyridin-2-yl)methanol (0.028 g, 1.1 eq.) and 1,1′-carbonyldiimidazole (0.053 g, 1.4 eq.) were mixed in dry dimethylformamide (1 mL). The reaction mixture was sealed and heated for 8 hours at 60° C. Then, the mixture was cooled to the ambient temperature and 1-[4-(difluoromethyl)benzenesulfonyl]-1,2-dihydrospiro[indole-3,4′-piperidine] (0.088 g, 1 eq.) was added in one portion. The reaction mixture was stirred for 16 hours at 60° C., and cooled to ambient temperature. The solvent was evaporated under reduced pressure and the residue was dissolved in DMSO (1 mL). The solution was filtered, and subjected to HPLC purification (deionized water/HPLC-grade acetonitrile) to give pyridin-2-ylmethyl 1-((4-(difluoromethyl)phenyl)sulfonyl)spiro[indoline-3,4′-piperidine]-1′-carboxylate (I-141). Yield: 42.9 mg, 34.2%; Appearance: Beige solid; ¹H NMR (400 MHz, DMSO-d₆) δ 8.50 (d, J=4.8 Hz, 1H), 7.96 (d, J=8.0 Hz, 2H), 7.78-7.66 (m, 3H), 7.54 (d, J=8.1 Hz, 1H), 7.33 (d, J=7.8 Hz, 1H), 7.28-6.77 (m, 5H), 5.13 (s, 2H), 4.15-3.94 (m, 2H), 3.91 (s, 2H), 3.00-2.84 (m, 2H), 1.75-1.58 (m, 2H), 1.22 (d, J=13.4 Hz, 2H): HPLC purity: 100%; LCMS Calculated for C₂₆H₂₅F₂N₃O₄S: 513.56; Observed: 514.2[M+H]⁺.

### Example 52—Synthesis of N-cyclopropyl-1-[4-(difluoromethyl)benzenesulfonyl]-1,2-dihydrospiro[indole-3,4′-piperidine]-1′-carboxamide (I-142)

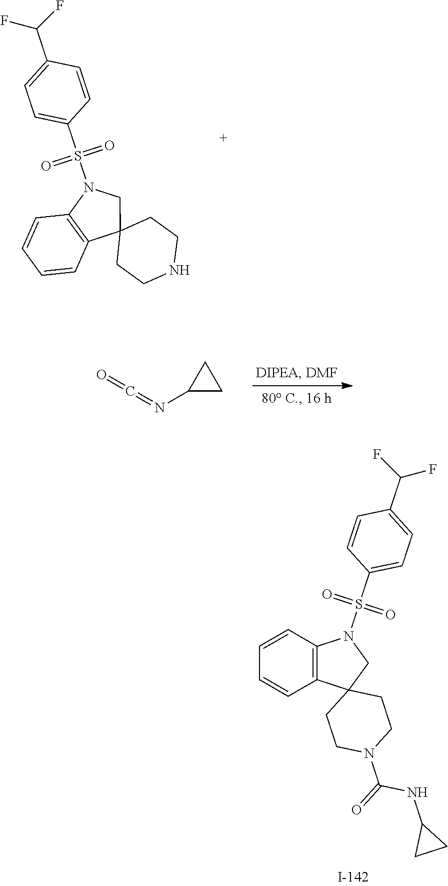 [SMILES: O=S(=O)(c1ccc(C(F)F)cc1)N1CC2(CCNCC2)c2ccccc21 ; O=C=NC1CC1 ; O=C(NC1CC1)N1CCC2(CC1)CN(S(=O)(=O)c1ccc(C(F)F)cc1)c1ccccc12]

1-[4-(difluoromethyl)benzenesulfonyl]-1,2-dihydrospiro[indole-3,4′-piperidine] (0.098 g, 1 eq.), isocyanatocyclopropane (0.024 g, 1.1 eq.), and N,N-diisopropylethylamine (0.067 g, 2 eq.) were mixed in dry N,N-dimethylformamide (1 mL), and the mixture was heated for 16 hours at 80° C. Afterwards, it was cooled to ambient temperature, and the solvent was evaporated under reduced pressure. The residue was dissolved in the DMSO (1 mL), and the solution was filtered and subjected to HPLC purification (deionized water/HPLC-grade methanol) to give N-cyclopropyl-1-[4-(difluoromethyl)benzenesulfonyl]-1,2-dihydrospiro[indole-3,4′-piperidine]-1′-carboxamide (I-142). Yield: 56.6 mg, 45.1%, Appearance: Yellow solid; ¹H NMR (600 MHz, DMSO-d₆) δ 7.98 (d, J=8.4 Hz, 2H), 7.75 (d, J=7.8 Hz, 2H), 7.48 (d, J=8.1 Hz, 1H), 7.26-7.20 (m, 1H), 7.20-6.98 (m, 3H), 6.55 (s, 1H), 3.82-3.77 (m, 2H), 2.73-2.66 (m, 2H), 1.53-1.42 (m, 2H), 1.07-0.99 (m, 2H), 0.53-0.47 (m, 2H), 0.36-0.30 (m, 2H); HPLC purity: 95.03%; LCMS Calculated for C₂₃H₂₅F₂N₃O₃S: 461.53; Observed: 462.2[M+H]⁺.

### Example 53—Synthesis of N-benzyl-1′-((4-(difluoromethyl)phenyl)sulfonyl)spiro[cyclohexane-1,3′-indoline]-4-carboxamide (I-143)

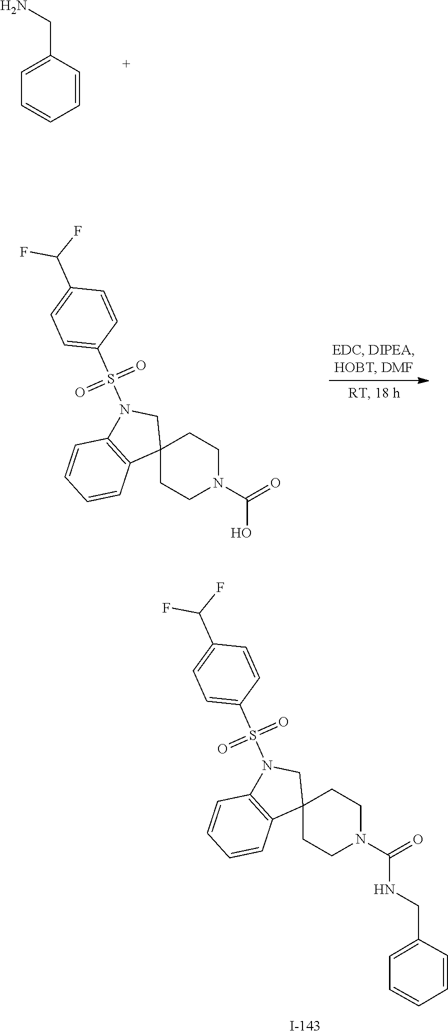 [SMILES: NCc1ccccc1 ; O=C(O)N1CCC2(CC1)CN(S(=O)(=O)c1ccc(C(F)F)cc1)c1ccccc12 ; O=C(NCc1ccccc1)N1CCC2(CC1)CN(S(=O)(=O)c1ccc(C(F)F)cc1)c1ccccc12]

1-Phenylmethanamine (0.02 g, 1.1 eq.), 1′-((4-(difluoromethyl)phenyl)sulfonyl)spiro[cyclohexane-1,3′-indoline]-4-carboxylic acid (0.072 g, 1 eq.), N,N-diisopropylethylamine (0.055 g, 2.5 eq.), 1-ethyl-3-(3-dimethylaminopropyl)carbodiimide (0.029 g, 1.05 eq.), and 1-hydroxy-7-azabenzotriazole (0.024 g, 1.1 eq.) were mixed in dry DMF (1 mL). The reaction mixture was stirred at ambient temperature for 18 hours. Then, the solvent was evaporated under reduced pressure and the residue was dissolved in DMSO (1 mL). The solution was filtered, analyzed by LCMS, and subjected to HPLC purification (deionized water/HPLC-grade methanol) to give N-benzyl-1′-((4-(difluoromethyl)phenyl)sulfonyl)spiro[cyclohexane-1,3′-indoline]-4-carboxamide (I-143). Yield: 30.3 mg, 30.2%; Appearance: Yellow solid; ¹H NMR (400 MHz, DMSO-d₆) δ 8.14-8.06 (m, 1H), 7.98-7.86 (m, 2H), 7.72 (d, J=8.0 Hz, 2H), 7.53 (d, J=8.2 Hz, 1H), 7.34-7.18 (m, 6H), 7.18-6.79 (m, 3H), 4.32-4.23 (m, 2H), 3.83-3.65 (m, 2H), 2.41-2.12 (m, 1H), 1.94-1.82 (m, 1H), 1.77-1.42 (m, 5H), 1.32-1.15 (m, 2H); HPLC purity: 100%; LCMS Calculated for C₂₈H₂₈F₂N₂O₃S: 510.6; Observed: 511.2[M+H]⁺.

### Example 54—Synthesis of N,N-dimethyl-4-{[1-(2,2,2-trifluoroethyl)-1′,2′-dihydrospiro[piperidine-4,3′-pyrazolo[1,5-alimidazol]-1′-yl]sulfonyl}benzene-1-sulfonamide (I-144)

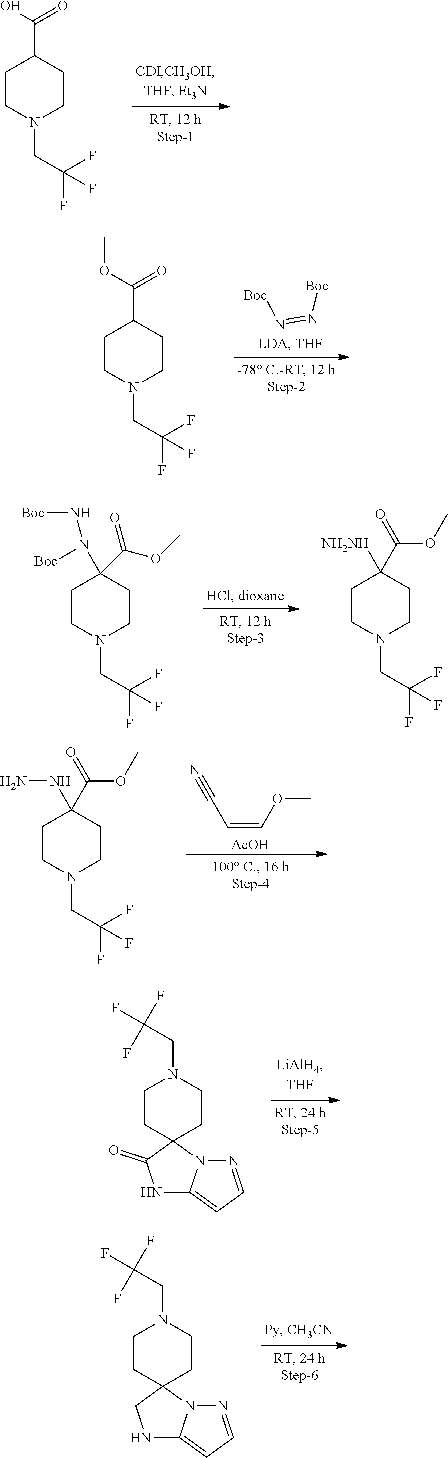

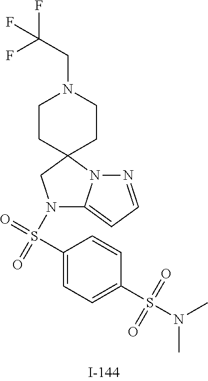 [SMILES: CN(C)S(=O)(=O)c1ccc(S(=O)(=O)N2CC3(CCN(CC(F)(F)F)CC3)n3nccc32)cc1]

### Step-1. Synthesis of methyl 1-(2,2,2-trifluoroethyl)piperidine-4-carboxylate

Carbonyldiimidazole (3.58 g, 22.1 mmol) was added to a solution of triethylamine (6.10 g, 60.3 mmol) and 1-(2,2,2-trifluoroethyl)piperidine-4-carboxylic acid hydrochloride (5 g, 20.1 mmol) in dry THF (100 mL). The mixture was refluxed for 2 hours. Afterwards, methanol (6.44 g, 201 mmol) was added to the boiling mixture and refluxing was continued for additional 6 hours. The mixture was then concentrated under reduced pressure. The residue was dissolved in ethyl acetate (150 mL) and filtered. The filtrate was washed with water (150 mL), dried over anhydrous sodium sulfate, filtered, and concentrated under reduced pressure to afford methyl 1-(2,2,2-trifluoroethyl)piperidine-4-carboxylate (4 g, 17.7 mmol, 99.98% purity, 88.2% yield).

### Step-2. Synthesis of methyl 4-{[(tert-butoxy)carbonyl]({[(tert-butoxy)carbonyl]amino})amino}-1-(2,2,2-tri fluoroethyl)piperidine-4-carboxylate

2.5 M n-BuLi solution in hexane (7.76 mL, 19.4 mmol) was added at −78° C. to a solution of diisopropylamine (1.96 g, 19.4 mmol) in dry THF (100 mL). The mixture was stirred for 10 min, and a solution of methyl 1-(2,2,2-trifluoroethyl) piperidine-4-carboxylate (4 g, 17.7 mmol) in dry THF (20 mL) was added dropwise to it at the same temperature. The mixture was stirred for 3 h at −78° C. Afterwards, a solution of di-tert-butyl azodicarboxylate (4.46 g, 19.4 mmol) in dry THF (50 mL) was added. The reaction mixture was warmed to room temperature, stirred overnight, and concentrated under reduced pressure. The residue was dissolved in ethyl acetate (150 mL) and filtered. The filtrate was washed with water (150 mL), dried over anhydrous sodium sulfate, filtered, and concentrated under reduced pressure to afford methyl 4-{[(tert-butoxy)carbonyl]({[(tert-butoxy)carbonyl]amino})amino}-1-(2,2,2-trifluoroethyl)piperidine-4-carboxylate (7 g, 15.3 mmol, 80% purity, 69.4% yield) that was used in the next step without further purification.

### Step-3. Synthesis of methyl 4-hydrazinyl-1-(2,2,2-trifluoroethyl)piperidine-4-carboxylate trishydrochloride

A 4 M solution of HCl in dioxane (31 mL) was added to a solution of methyl 4-{[(tert-butoxy)carbonyl]({[(tert-butoxy)carbonyl]amino})amino}-1-(2,2,2-trifluoroethyl)piperidine-4-carboxylate (7 g, 12.2 mmol) in dioxane (50 mL), and the mixture was stirred for 24 h at room temperature. Afterwards, it was concentrated under reduced pressure to afford methyl 4-hydrazinyl-1-(2,2,2-trifluoroethyl)piperidine-4-carboxylate trishydrochloride (4 g, 10.9 mmol, 75% purity, 96.4% yield) that was used in the next step without further purification.

### Step-4. Synthesis of 1-(2,2,2-trifluoroethyl)-1′,2′-dihydrospiro[piperidine-4,3′-pyrazolo[1,5-a]imidazol]-2′-one

(2E)-3-methoxyprop-2-enenitrile (0.972 g, 11.7 mmol) was added to a suspension of methyl 4-hydrazinyl-1-(2,2,2-trifluoroethyl)piperidine-4-carboxylate trishydrochloride (4 g, 11.7 mmol) in acetic acid (50 mL), and the resulting mixture was heated under reflux for 12 hours. Afterwards, the reaction mixture was cooled to room temperature and evaporated to dryness in vacuo. The crude product was purified by flash chromatography (chloroform/acetonitrile) to give 1-(2,2,2-trifluoroethyl)-1′,2′-dihydrospiro[piperidine-4,3′-pyrazolo[1,5-a]imidazol]-2′-one as a white solid (0.4 g, 1.45 mmol, 90,% purity, 11.2% yield).

### Step-5. Synthesis of 1-(2,2,2-trifluoroethyl)-1′,2′-dihydrospiro[piperidine-4,3′-pyrazolo[1,5-a]imidazole]

A solution of 1-(2,2,2-trifluoroethyl)-1′,2′-dihydrospiro[piperidine-4,3′-pyrazolo[1,5-a]imidazol]-2′-one (0.4 g, 1.31 mmol) in tetrahydrofuran (25 mL) was added dropwise at −5° C. to a suspension of lithium aluminum hydride (0.099 g, 2.62 mmol) in tetrahydrofuran (5 mL). After addition, the solution was warmed to room temperature and stirred for 12 hours. The solution was quenched with a mixture of water/tetrahydrofuran (1/1.5 mL). The resulting mixture was filtered, and the filtrate was evaporated under reduced pressure to give 1-(2,2,2-trifluoroethyl)-1′,2′-dihydrospiro[piperidine-4,3′-pyrazolo[1,5-a]imidazole] (0.35 g, 1.34 mmol, 91.35% purity, 93.8% yield).

### Step-6. Synthesis of N,N-dimethyl-4-([1-(2,2,2-trifluoroethyl)-1′,2′-dihydrospiro[piperidine-4,3′-pyrazolo[1,5-a]imidazol]-1′-yl]sulfonyl)benzene-1-sulfonamide

4-(Dimethylsulfamoyl)benzene-1-sulfonyl chloride (0.417 g, 1.47 mmol) was added to a mixture of 1-(2,2,2-trifluoroethyl)-1′,2′-dihydrospiro[piperidine-4,3′-pyrazolo[1,5-a]imidazole](0.35 g, 1.22 mmol) and pyridine (0.965 g, 12.2 mmol) in dry acetonitrile (50 mL). The reaction mixture was stirred at room temperature overnight and evaporated. The residue was subjected to HPLC purification (deionized water/HPLC-grade acetonitrile) to afford the product N,N-dimethyl-4-{[1-(2,2,2-trifluoroethyl)-1′,2′-dihydrospiro[piperidine-4,3′-pyrazolo[1,5-a]imidazol]-1′-yl]sulfonyl}benzene-1-sulfonamide (I-144). Yield: 40.6 mg, 6.21%; Appearance: Beige solid; ¹H NMR (600 MHz, DMSO-d₆) δ 12.21 (s, 1H), 7.39-7.36 (m, 2H), 7.33 (d, J=3.2 Hz, 1H), 7.15-7.12 (m, 2H), 3.32 (s, 3H), 3.28 (d, J=6.7 Hz, 2H), 1.87 (t, J 6.8 Hz, 2H), 1.25 (s, 9H); HPLC purity: 100%; LCMS Calculated for C₁₉H₂₄F₃N₅O₄S₂: 507.55; Observed: 508.2[M+H]⁺.

### Example 55—Synthesis of cyclopropylmethyl 1′-[4-(dimethylsulfamoyl)benzenesulfonyl]-1′,2′-dihydrospiro[pipridine-4,3′-pyrazolo[1,5-a]imidazole]-1-carboxylate (I-145)

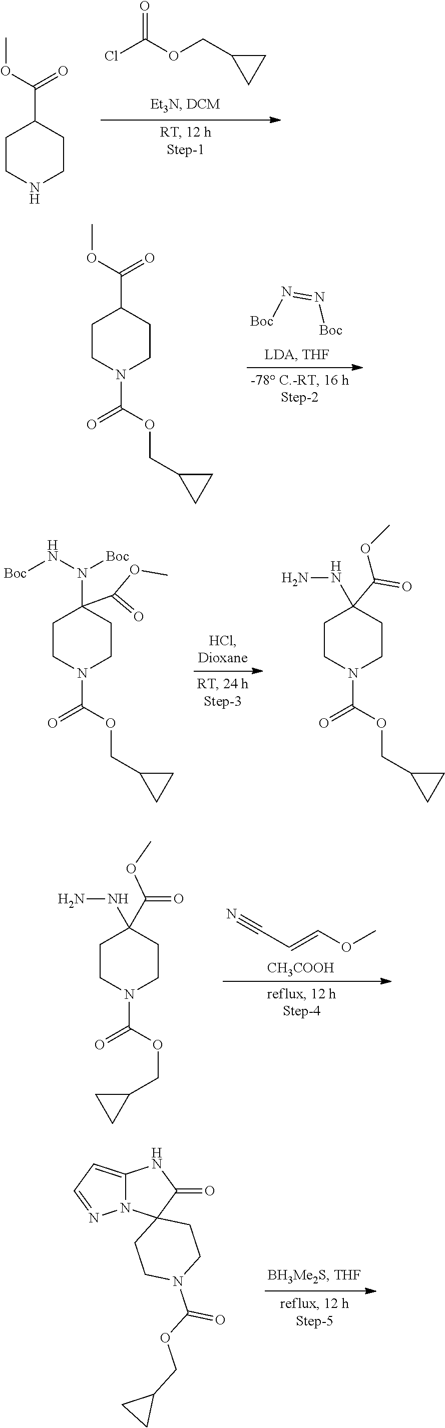

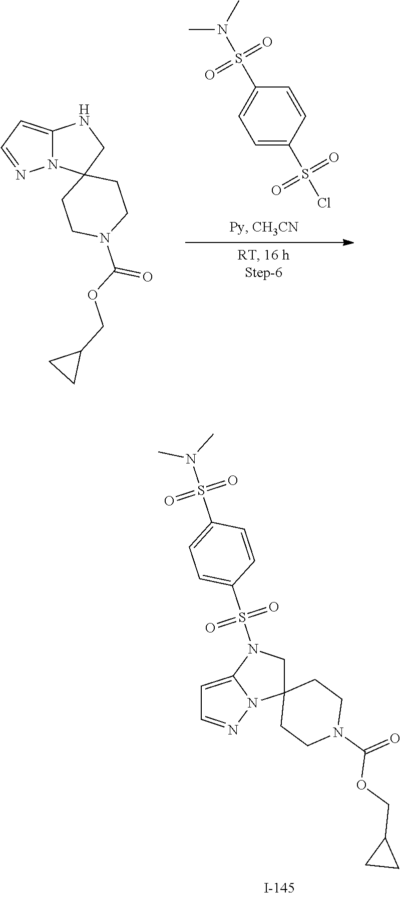 [SMILES: O=C(OCC1CC1)N1CCC2(CC1)CNc1ccnn12 ; CN(C)S(=O)(=O)c1ccc(S(=O)(=O)Cl)cc1 ; CN(C)S(=O)(=O)c1ccc(S(=O)(=O)N2CC3(CCN(C(=O)OCC4CC4)CC3)n3nccc32)cc1]

### Step-1. Synthesis of 1-cyclopropylmethyl 4-methyl piperidine-1,4-dicarboxylate

Cyclopropylmethyl carbonochloridate (5.15 g, 38.3 mmol) was added to a solution of triethylamine (3.87 g, 38.3 mmol) and methyl piperidine-4-carboxylate (5 g, 34.9 mmol) in dry dichloromethane (100 mL). The mixture was stirred for 12 hours at room temperature. Afterwards, the mixture was concentrated under reduced pressure. The residue was dissolved in ethyl acetate (150 mL) and filtered, and the filtrate was washed with water (150 mL), dried over anhydrous sodium sulfate, filtered, and concentrated under reduced pressure to afford 1-cyclopropylmethyl 4-methyl piperidine-1,4-dicarboxylate (8 g, 33.1 mmol, 100% purity, 95.0% yield).

### Step-2. Synthesis of 4-methyl 4-{[(tert-butoxy)carbonyl]({[(tert-butoxy)carbonyl]amino})amino}piperidine-1,4-dicarboxylate

A 2.5 M n-BuLi solution in hexane (14.5 mL, 36.4 mmol) was added at −78° C. to a solution of diisopropylamine (3.68 g, 36.4 mmol) in dry THF (100 mL). The mixture was stirred for 10 min, and a solution of 1-cyclopropylmethyl 4-methyl piperidine-1,4-dicarboxylate (8 g, 33.1 mmol) in dry THF (20 mL) was added dropwise to it at the same temperature. The mixture was stirred for 3 h at −78° C. Then, a solution of di-tert-butyl azodicarboxylate (7.99 g, 34.7 mmol) in dry THF (50 mL) was added. The reaction mixture was warmed to room temperature, stirred overnight, and concentrated under reduced pressure. The residue was dissolved in ethyl acetate (150 mL) and filtered, and the filtrate was washed with water (150 mL), dried over anhydrous sodium sulfate, filtered, and concentrated under reduced pressure to afford 1-cyclopropylmethyl 4-methyl 4-{[(tert-butoxy)carbonyl]({[(tert-butoxy)carbonyl]amino})amino}piperidine-1,4-dicarboxylate (12 g, 25.4 mmol, 90% purity, 69.2% yield) that was used in the next step without further purification.

### Step-3. Synthesis of 4-methyl 4-hydrazinylpiperidine-1,4-dicarboxylate bishydrochloride

A 4 M solution of HCl in dioxane (50 mL) was added to a solution of 1-cyclopropylmethyl 4-methyl 4-{[(tert-butoxy)carbonyl]({[(tert-butoxy)carbonyl]amino})amino}piperidine-1,4-dicarboxylate (12 g, 22.9 mmol) in dioxane (100 mL), and the mixture was stirred for 24 h at room temperature. Then, it was concentrated under the reduced pressure to afford 1-cyclopropylmethyl 4-methyl 4-hydrazinylpiperidine-1,4-dicarboxylate bishydrochloride (7 g, 12.2 mmol, 60% purity, 53.3% yield) that was used in the next step without further purification.

### Step-4. Synthesis of cyclopropylmethyl 2′-oxo-1′,2′-dihydrospiro[piperidine-4,3′-pyrazolo[1,5-a]imidazole]-1-carboxylate

(2E)-3-methoxyprop-2-enenitrile (1.27 g, 15.4 mmol) was added to a suspension of 1-cyclopropylmethyl 4-methyl 4-hydrazinylpiperidine-1,4-dicarboxylate (7 g, 15.4 mmol) in an acetic acid (100 mL), and the resulting mixture was heated under reflux for 12 hours. Then, it was cooled to room temperature and evaporated to dryness in vacuo. The crude product was purified by flash chromatography (chloroform/acetonitrile) to give cyclopropylmethyl 2′-oxo-1′,2′-dihydrospiro[piperidine-4,3′-pyrazolo[1,5-a]imidazole]-1-carboxylate as a white solid (0.8 g, 15.4 mmol, 95% purity, 17.0% yield).

### Step-5. Synthesis of cyclopropylmethyl 1′,2′-dihydrospiro[piperidine-4,3′-pyrazolo[1,5-a]imidazole]-1-carboxylate

A solution of cyclopropylmethyl 2′-oxo-1′,2′-dihydrospiro[piperidine-4,3′-pyrazolo[1,5-a]imidazole]-1-carboxylate (0.8 g, 2.61 mmol) in tetrahydrofuran (25 mL) was added dropwise at −5° C. to borane dimethyl sulfide complex (0.587 g, 7.83 mmol). After addition, the solution was warmed to room temperature and refluxed for 12 hours. The solution was cooled to room temperature, quenched with methanol (5 mL), and filtered, and the filtrate was evaporated under reduced pressure to give cyclopropylmethyl 1′,2′-dihydrospiro[piperidine-4,3′-pyrazolo[1,5-a]imidazole]-1-carboxylate (0.3 g, 1.08 mmol, 100% purity, 41.6% yield).

### Step-6. Synthesis of cyclopropylmethyl 1′-[4-(dimethylsulfamoyl)benzenesulfonyl]-1′,2′-dihydrospiro[piperidine-4,3′-pyrazolo[1,5-a]imidazole]-1-carboxylate

4-(Dimethylsulfamoyl)benzene-1-sulfonyl chloride (0.169 g, 0.596 mmol) was added to the mixture of cyclopropylmethyl 1′,2′-dihydrospiro[piperidine-4,3′-pyrazolo[1,5-a]imidazole]-1-carboxylate (0.15 g, 0.542 mmol) and pyridine (0.213 g, 2.7 mmol) in dry acetonitrile (50 mL). The reaction mixture was stirred at room temperature overnight and evaporated. The residue was subjected to HPLC purification (deionized water/HPLC-grade acetonitrile) to afford the product cyclopropylmethyl 1′-[4-(dimethylsulfamoyl)benzenesulfonyl]-1′,2′-dihydrospiro[piperidine-4,3′-pyrazolo[1,5-a]imidazole]-1-carboxylate (I-145). Yield: 21.6 mg, 7.24%; Appearance: Colorless oil, ¹H NMR (400 MHz, DMSO-d₆) δ 8.11 (d, J=8.2 Hz, 2H), 7.99 (d, J=8.2 Hz, 2H), 7.45 (d, J=1.9 Hz, 1H), 6.01 (d, J=1.9 Hz, 1H), 4.29 (s, 2H), 3.83 (d, J=7.2 Hz, 2H), 3.67 (d, J=14.7 Hz, 2H), 3.30 (s, 2H), 2.63 (s, 6H), 1.54 (dd, J=9.0, 4.0 Hz, 2H), 1.43 (s, 2H), 1.06 (q, J=6.2, 5.0 Hz, 1H), 0.52-0.42 (m, 2H), 0.23 (dt, J=6.3, 3.1 Hz, 2H); HPLC purity: 100%; LCMS Calculated for C₂₂H₂₉N₅O₆S₂: 523.63; Observed: 524.4 [M+H]⁺.

### Example 56—Synthesis of N,N-dimethyl-4-({6′-methyl-1′,2′-dihydrospiro[cyclohexane-1,3′-pyrazolo[1,5-a]imidazol]-1′-yl}sulfonyl)benzene-1-sulfonoimidamide (I-146)

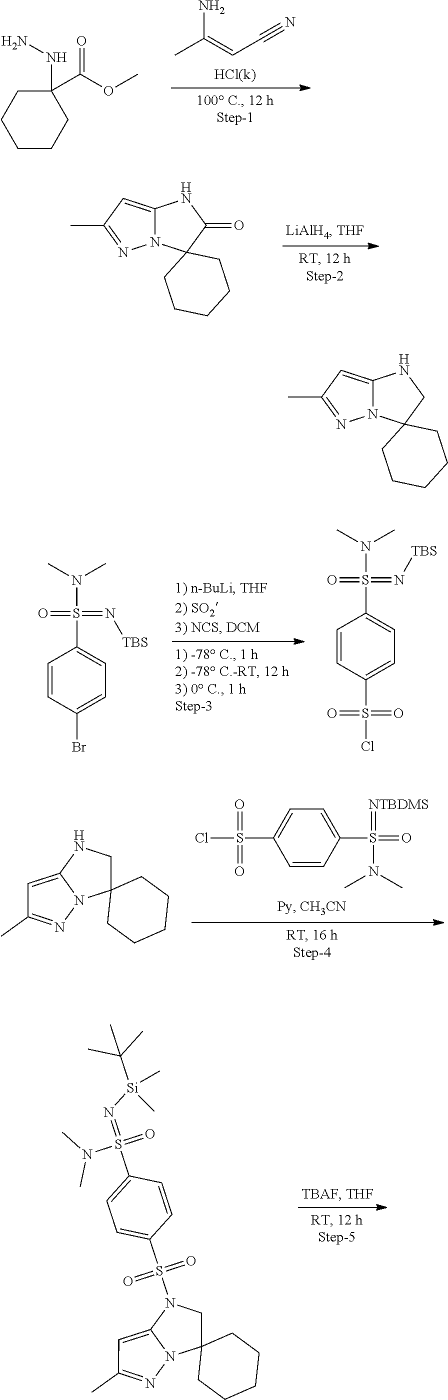

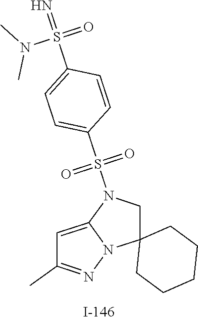 [SMILES: Cc1cc2n(n1)C1(CCCCC1)CN2S(=O)(=O)c1ccc(S(=N)(=O)N(C)C)cc1]

### Step-1. Synthesis of 6′-methylspiro[cyclohexane-1,3′-imidazo[1,2-b]pyrazol]-2′(1′H)-one

(2Z)-3-aminobut-2-enenitrile (1.05 g, 12.75 mmol) was added to a suspension of methyl 1-hydrazinylcyclohexane-1-carboxylate dihydrochloride (3.12 g, 12.75 mmol) in a 3 M HCl/H₂O mixture (⅓, 36 mL), and the resulting mixture was heated under reflux for 12 hours. Then it was cooled to room temperature and neutralized with 2.5M sodium hydroxide aq. solution. The suspension was extracted with dichloromethane (30 mL×3). The combined organic layers were washed with brine (20 mL), dried over sodium sulfate, and evaporated in vacuo to give 6′-methylspiro[cyclohexane-1,3′-imidazo[1,2-b]pyrazol]-2′(1′H)-one as a white solid (1.05 g, 5.10 mmol, 100% purity, 40% yield).

### Step-2. Synthesis of 6′-methyl-1′,2′-dihydrospiro[cyclohexane-1,3′-pyrazolo[1,5-a]imidazole]

A solution of 6′-methyl-1′,2′-dihydrospiro[cyclohexane-1,3′-pyrazolo[1,5-a]imidazol]-2′-one (1.05 g, 5.10 mmol) in tetrahydrofuran (75 mL) was added dropwise at −5° C. to a suspension of lithium aluminum hydride (0.237 g, 6.09 mmol) in tetrahydrofuran (15 mL). After addition, the solution was warmed to room temperature and stirred for 12 hours. The solution was quenched with a mixture of water/tetrahydrofuran (3/4.5 mL). The resulting mixture was filtered, and the filtrate was evaporated under reduced pressure to give 6′-methyl-1′,2′-dihydrospiro[cyclohexane-1,3′-pyrazolo[1,5-a]imidazole] (0.33 g, 1.725 mmol, 100% purity, 33.8% yield).

### Step-3. Synthesis of 4-[(tert-butyldimethylsilyl)dimethyl-S-aminosulfonimidoyl]benzene-1-sulfonyl chloride

A 2.5 M solution of n-butyllithium (0.382 g, 5.97 mmol) in hexane (2.38 mL) was added to a solution of 4-bromo-N′-(tert-butyldimethylsilyl)-N,N-dimethylbenzenesulfonimidamide (1.88 g, 4.98 mmol) in anhydrous tetrahydrofuran (20 mL) at −78° C. under argon atmosphere, and the mixture was stirred for 1 h at −78° C. Afterwards, a solution of SO₂ (0.954 g, 14.9 mmol) in tetrahydrofuran (20 mL) was added to the resulting mixture at the same temperature. Then, the cooling bath was removed, and the mixture was allowed to warm to room temperature and stir for 12 h. The solution was then evaporated in vacuo. The residue was dissolved in dichloromethane (20 mL), and N-chlorosuccinimide (0.797 g, 5.97 mmol) was added, maintaining the reaction mixture temperature at 0° C. The mixture was stirred for 30 minutes, and then diluted with ethyl acetate (20 mL) and water (20 mL). The organic layer was separated, dried over sodium sulfate, and filtered, and the filtrate was evaporated in vacuo to give 4-[(tert-butyldimethylsilyl)dimethyl-S-aminosulfonimidoyl]benzene-1-sulfonyl chloride as a dark resin (1.76 g, 4.43 mmol, 58.66% purity, 52.2% yield) that was used in the next step without further purification.

### Step-4. Synthesis of N-(tert-butyldimethylsilyl)-N,N-dimethyl-4-({6′-methyl-1′,2′-dihydrospiro[cyclohexane-1,3′-pyrazolo[1,5-a]imidazol]-1′-yl}sulfonyl)benzene-1-sulfonoimidamide

4-(Difluoromethyl)benzene-1-sulfonyl chloride (0.387 g, 1.71 mmol) was added to a mixture of 6′-methyl-1′,2′-dihydrospiro[cyclohexane-1,3′-pyrazolo[1,5-a]imidazole] (0.3 g, 1.56 mmol) and pyridine (0.6 g, 7.80 mmol) in dry acetonitrile (25 mL). The reaction mixture was stirred at room temperature overnight and evaporated. The residue was subjected to HPLC purification (deionized water/HPLC-grade acetonitrile) to afford 1′-[4-(difluoromethyl)benzenesulfonyl]-6′-methyl-1′,2′-dihydrospiro[cyclohexane-1,3′-pyrazolo[1,5-a]imidazole] as white solid (0.2882 g, 0.755 mmol, 95% purity, 45.8% yield). The analytical data provided for this compound provisionally supports the proposed structure for 1′-[4-(difluoromethyl)benzenesulfonyl]-6′-methyl-1′,2′-dihydrospiro[cyclohexane-1,3′-pyrazolo[1,5-a]imidazole].

### Step-5. Synthesis of N,N-dimethyl-4-((6′-methylspiro[cyclohexane-1,3′-imidazo[1,2-b]pyrazol]-1′(2′H)-yl)sulfonyl)benzenesulfonimidamide

A 1 M solution of TBAF in THF (1.55 mL, 0.405 g, 1.55 mmol) was added to a solution of N′-(tert-butyldimethylsilyl)-N,N-dimethyl-4-((6′-methylspiro[cyclohexane-1,3′-imidazo[1,2-b]pyrazol]-1′(2′H)-yl)sulfonyl)benzenesulfonimidamide (0.86 g, 1.55 mmol) in dry THF (20 mL) under argon atmosphere. The reaction mixture was stirred for 12 h and evaporated in vacuo. The residue was subjected to HPLC purification (deionized water/HPLC-grade acetonitrile) to afford N,N-dimethyl-4-({6′-methyl-1′,2′-dihydrospiro[cyclohexane-1,3′-pyrazolo[1,5-a]imidazol]-1′-yl}sulfonyl)benzene-1-sulfonoimidamide (I-146). Yield: 81 mg, 11.3%; Appearance: Yellow solid; 1H NMR (400 MHz, DMSO-d₆) δ 8.07 (d, J=8.2 Hz, 2H), 7.98 (d, J=8.3 Hz, 2H), 5.78 (s, 1H), 4.76 (s, 1H), 4.12 (s, 2H), 2.54 (s, 6H), 2.12 (s, 3H), 1.62 (d, J=11.5 Hz, 2H), 1.50 (q, J=18.7, 15.1 Hz, 3H), 1.20 (dq, J=54.3, 13.2, 12.4 Hz, 5H); HPLC purity: 100%; LCMS Calculated for C₁₉H₂₇N₅O₃S₂: 437.58; Observed: 438.2 [M+H]⁺.

### Example 57

The following compounds were prepared using standard chemical manipulations and procedures similar to those used for the preparation of the previous example.

| Compound No. | Structure | Analytical Data |
|---|---|---|
| I-147 | 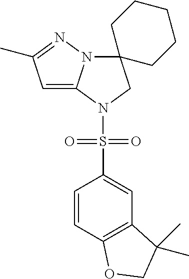 | Yield: 108.6 mg, 16.4%; Appearance: Yellow solid; ¹H NMR (600 MHz, DMSO-d₆) δ 7.72 (d, J = 2.1 Hz, 1H), 7.56 (dd, J = 8.5, 2.0 Hz, 1H), 6.93 (d, J = 8.5 Hz, 1H), 5.72 (s, 1H), 4.32 (s, 2H), 4.06 (s, 2H), 2.08 (s, 3H), 1.60 (dt, J = 13.5, 4.0 Hz, 2H), 1.50 (td, J = 12.4, 3.9 Hz, 2H), 1.46- 1.42 (m, 1H), 1.25 (s, 8H), 1.15 (t, J = 11.8 Hz, 1H), 1.07 (d, J = 12.9 Hz, 2H); HPLC purity: 100%; LCMS Calculated for C₂₁H₂₇N₃O₃S: 401.21; Observed: 402.2 [M + H]⁺. |
| I-148 | 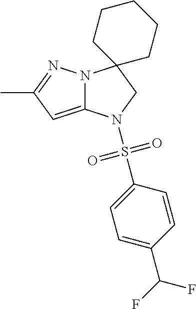 [SMILES: Cc1cc2n(n1)C1(CCCCC1)CN2S(=O)(=O)c1ccc(C(F)F)cc1] | Yield: 288.2 mg, 45.8%; Appearance: White solid; ¹H NMR (600 MHz, DMSO-d₆) δ 8.01 (d, J = 8.1 Hz, 2H), 7.81 (d, J = 8.1 Hz, 2H), 7.13 (t, J = 55.3 Hz, 1H), 5.74 (s, 1H), 2.09 (s, 3H), 1.61 (dt, J = 13.9, 4.3 Hz, 2H), 1.52 (td, J = 12.3, 3.9 Hz, 2H), 1.47-1.42 (m, 1H), 1.31-1.21 (m, 2H), 1.15 (d, J = 13.4 Hz, 3H); HPLC purity: 100%; LCMS Calculated for C₁₈H₂₁F₂N₃O₂S: 381.44; Observed: 382.2 [M + H]⁺. |
| I-149 | 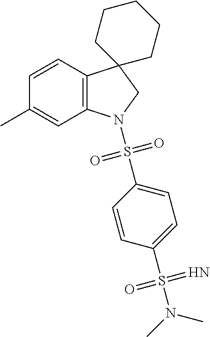 | Yield: 356.7 mg, 36.6%; Appearance: Yellow solid; ¹H NMR (400 MHz, DMSO-d₆) δ 8.06- 7.86 (m, 4H), 7.35 (s, 1H), 7.04 (d, J = 7.7 Hz, 1H), 6.87 (d, J = 7.6 Hz, 1H), 4.69 (s, 1H), 3.78 (s, 2H), 2.50 (s, 6H), 2.33 (s, 3H), 1.59 (d, J = 12.2 Hz, 1H), 1.53-1.43 (m, 2H), 1.41-1.06 (m, 5H), 0.94 (t, J = 12.4 Hz, 2H); HPLC purity: 100%; LCMS Calculated for C₂₂H₂₉N₃O₃S₂: 447.61; Observed: 448.2 [M + H]⁺. |

### Example 58—Synthesis of 4-(1-{1′,2′-dihydrospiro[cyclohexane-1,3′-indol]-1′-yl}ethyl)-N,N-dimethylbenzene-1-sulfonamide (I-150)

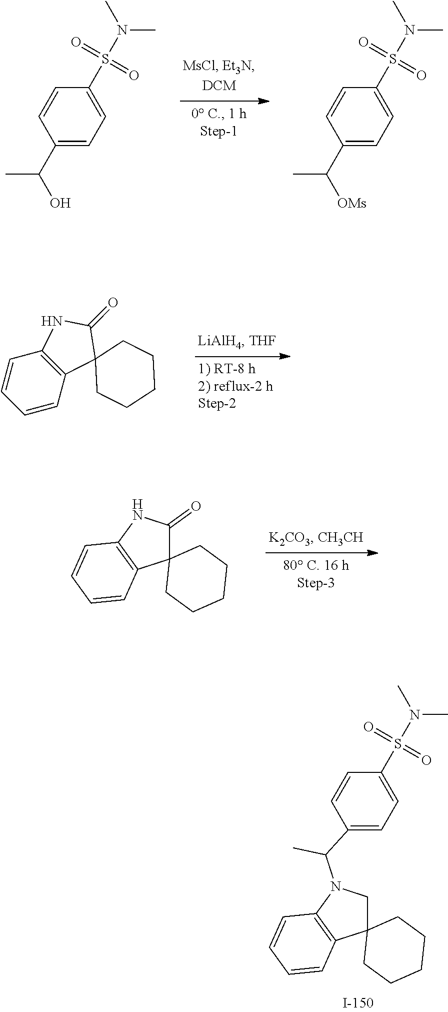 [SMILES: CC(O)c1ccc(S(=O)(=O)N(C)C)cc1 ; CC(OS(C)(=O)=O)c1ccc(S(=O)(=O)N(C)C)cc1 ; O=C1Nc2ccccc2C12CCCCC2 ; O=C1Nc2ccccc2C12CCCCC2 ; CC(c1ccc(S(=O)(=O)N(C)C)cc1)N1CC2(CCCCC2)c2ccccc21]

### Step-1. Synthesis of 1-[4-(dimethylsulfamoyl)phenyl]ethyl methanesulfonate

Triethylamine (0.164 g, 1.63 mmol) was added to a solution of 4-(1-hydroxyethyl)-N,N-dimethylbenzenesulfonamide (0.250 g, 1.09 mmol) in dichloromethane (10 mL). The solution was cooled in an ice-water bath and methanesulfonyl chloride (0.148 g, 1.30 mmol) was added dropwise. The mixture was stirred for 1 h at 0° C. and poured into water (20 mL). The layers were separated and the aqueous phase was extracted with dichloromethane (10 mL). The combined organic extracts were washed successively with saturated NaHCO₃ aq. solution (10 mL) and brine (10 mL), then dried over sodium sulfate, filtered, and evaporated under reduced pressure to afford 1-[4-(dimethylsulfamoyl)phenyl]ethyl methanesulfonate as light-yellow solid (0.325 g, 1.05 mmol, 95% purity, 91.9% yield).

### Step-2. Synthesis of 1′,2′-dihydrospiro[cyclohexane-1,3′-indole]

A solution of 1′,2′-dihydrospiro[cyclohexane-1,3′-indol]-2′-one (0.55 g, 2.73 mmol) in THF (1 mL) was added to a suspension of LiAlH₄ (0.117 g, 3.09 mmol) in THF (10 mL). The reaction mixture was stirred at room temperature for 8 h, and then refluxed for 2 h. The mixture was cooled to room temperature and carefully quenched with H₂O (0.12 mL), then with 6 N NaOH aq. solution (0.12 mL). The mixture was then diluted with water (5 mL). The resulting slurry was filtered; the filtrate was concentrated under reduced pressure and dissolved in ethyl acetate (20 mL). The organic layer was washed with brine (10 mL), dried over sodium sulfate, filtered, and concentrated under reduced pressure to afford 1′,2′-dihydrospiro[cyclohexane-1,3′-indole] (0.51 g, 2.58 mmol, 95% purity, 94.7% yield).

### Step-3. Synthesis of 4-[(3-cyclohexyl-4-fluoro-1H-indol-1-yl)sulfonyl]-N,N-dimethylbenzene-1-sulfonamide

Dipotassium carbonate (0.439 g, 3.18 mmol) was added to a solution of 1′,2′-dihydrospiro[cyclohexane-1,3′-indole] (0.2 g, 1.06 mmol) and 1-[4-(dimethylsulfamoyl)phenyl]ethyl methanesulfonate (0.325 g, 1.06 mmol) in acetonitrile (10 mL). The reaction mixture was stirred at 80° C. for 16 h. After completion (TLC control), the reaction mixture was diluted with water (10 mL) and extracted with ethyl acetate (20 mL×2). The combined organic extracts were washed with water (10 mL) and brine (10 mL), dried over anhydrous sodium sulfate, filtered, and concentrated under reduced pressure. The crude product was purified by HPLC (deionized water/HPLC-grade acetonitrile, ammonia) to provide 4-(1-{1′,2′-dihydrospiro[cyclohexane-1,3′-indol]-1′-yl}ethyl)-N,N-dimethylbenzene-1-sulfonamide (I-150). Yield: 67.6 mg, 15.2%; Appearance: Yellow solid; ¹H NMR (600 MHz, DMSO-d₆) δ 7.70 (dd, J=9.1, 7.4 Hz, 2H), 7.62 (d, J=8.3 Hz, 2H), 6.96 (dd, J=7.3, 1.3 Hz, 1H), 6.88 (td, J=7.6, 1.3 Hz, 1H), 6.55-6.47 (m, 1H), 6.29 (d, J=7.8 Hz, 1H), 4.84 (q, J=6.9 Hz, 1H), 3.30-3.19 (m, 2H), 2.57 (s, 6H), 1.66-1.53 (m, 6H), 1.51 (d, J=6.9 Hz, 3H), 1.44 (tt, J=13.0, 6.3 Hz, 1H), 1.36-1.18 (m, 3H); HPLC purity: 100%; LCMS Calculated for C₂₃H₁₀N₂O₂S: 398.56; Observed: 399.4 [M+H]⁺.

### Example 59—Synthesis of cyclopropyl(imino)(4-(spiro[cyclohexane-1,3′-indolin]-1′-ylsulfonyl)phenyl)-)λ⁶-sulfanone (I-151)

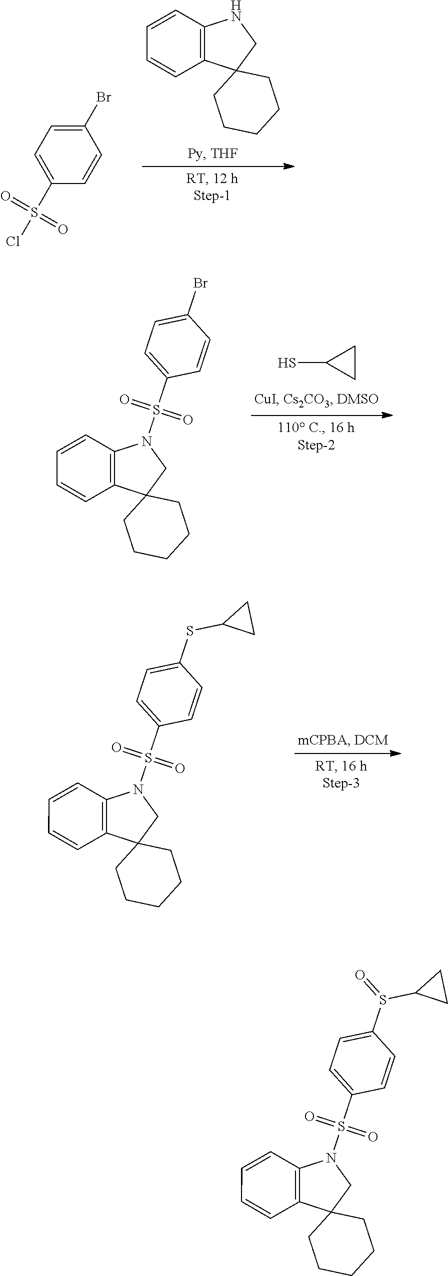

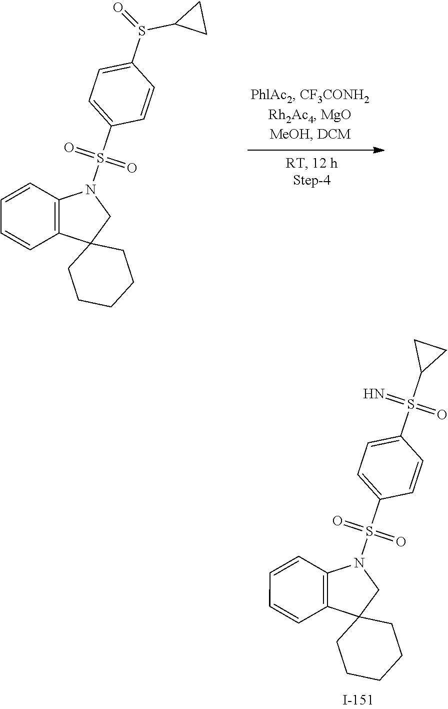 [SMILES: O=S(c1ccc(S(=O)(=O)N2CC3(CCCCC3)c3ccccc32)cc1)C1CC1 ; N=S(=O)(c1ccc(S(=O)(=O)N2CC3(CCCCC3)c3ccccc32)cc1)C1CC1]

### Step-1. Synthesis of benzyl 1′-(4-bromobenzenesulfonyl)-1′,2′-dihydrospiro[cyclohexane-1,3′-indole]

Pyridine (0.471 mL, 5.84 mmol) and 4-bromobenzene-1-sulfonyl chloride (1 g, 3.91 mmol) were added to a solution of benzyl 1′,2′-dihydrospiro[cyclohexane-1,3′-indole] (0.805 g, 4.3 mmol) in tetrahydrofuran (50 mL). The reaction mixture was stirred at room temperature for 12 hours. The solvent was evaporated under reduced pressure. Flash chromatography purification (hexane/chloroform/MTBE) of the residue afforded benzyl 1′-(4-bromobenzenesulfonyl)-1′,2′-dihydrospiro[cyclohexane-1,3′-indole] (1.4 g, 3.1 mmol, 90% purity, 79.7% yield).

### Step-2. Synthesis of 1′-((4-(cyclopropylthio)phenyl)sulfonyl)spiro[cyclohexane-1,3′-indoline]

A mixture of 1′-(4-bromobenzenesulfonyl)-1′,2′-dihydrospiro[cyclohexane-1,3′-indole](0.5 g, 1.23 mmol), cyclopropanethiol (0.136 g, 1.84 mmol), dicaesium (1+) carbonate (0.8 g, 2.46 mmol), and copper iodide (0.0584 g, 0.307 mmol) in DMSO (20 mL) was stirred at 110° C. overnight. The reaction mixture was then cooled to room temperature, diluted with water (40 mL), and extracted with ethyl acetate (50 mL×2). Combined layers were dried over sodium sulfate, filtered, and evaporated under reduced pressure. The residue was subjected to HPLC purification (deionized water/HPLC-grade acetonitrile) that afforded 1′-((4-(cyclopropylthio)phenyl)sulfonyl)spiro[cyclohexane-1,3′-indoline] (0.2 g, 0.5 mmol, 100% purity, 26% yield).

### Step-3. Synthesis of 1′-[4-(cyclopropanesulfinyl)benzenesulfonyl]-1′,2′-dihydrospiro[cyclohexane-1,3′-indole]

3-Chlorobenzene-1-carboperoxoic acid (0.0862 g, 0.5 mmol) was added to a solution of 1′-[4-(cyclopropylsulfanyl)benzenesulfonyl]-1′,2′-dihydrospiro[cyclohexane-1,3′-indole] (0.2 g, 0.5 mmol) in dichloromethane (20 mL). The mixture was stirred overnight, then washed with sodium bicarbonate sat. aq. solution (20 mL) and evaporated to dryness. The residue was subjected to HPLC purification (deionized water/HPLC-grade acetonitrile) to afford 1′-[4-(cyclopropanesulfinyl)benzenesulfonyl]-1′,2′-dihydrospiro[cyclohexane-1,3′-indole] (0.074 g, 0.178 mmol, 100% purity, 35.8% yield).

### Step-4. Synthesis of (cyclopropanesulfonimidoyl)phenyl)sulfonyl)spiro[cyclohexane-1,3′-indoline]

1′-((4-(cyclopropylsulfinyl)phenyl)sulfonyl)spiro[cyclohexane-1,3′-indoline] (0.0743 g, 0.178 mmol), trifluoroacetamide (0.0441 g, 0.391 mmol), iodobenzene diacetate (0.0943 g, 0.293 mmol), and magnesium oxide (0.0315 g, 0.783 mmol) were mixed in a methanol/dichloromethane mixture (1/1, 30 mL). The mixture was stirred for 5 minutes, treated with rhodium(II) acetate dimer (0.0039 g, 0.0089 mmol), and stirred at room temperature for 12 hours. The, the suspension was diluted with methanol (25 mL), treated with saturated solution of potassium carbonate (30 mL), and stirred at room temperature for 4 hours. Afterwards, the mixture was treated with water (50 mL), and the organic phase was separated and filtered through Celite. The aqueous phase was extracted with dichloromethane (30 mL×3). The solvent was evaporated under reduced pressure and the residue was subjected to HPLC purification (deionized water/HPLC-grade acetonitrile) to afford 1′-((4-(cyclopropanesulfonimidoyl)phenyl)sulfonyl)spiro[cyclohexane-1,3′-indoline] (I-151). Yield: 6.7 mg, 8.3%; Appearance: Colorless oil; ¹H NMR (600 MHz, CDCl₃) δ 8.08-7.96 (m, 4H), 7.64 (d, J=8.1 Hz, 1H), 7.22 (ddd, J=8.3, 6.4, 2.4 Hz, 1H), 7.08-7.03 (m, 2H), 3.77 (s, 2H), 2.55 (tt, J=7.9, 4.7 Hz, 1H), 1.70 (d, J=11.3 Hz, 1H), 1.68-1.62 (m, 2H), 1.49-1.38 (m, 3H), 1.32-1.22 (m, 5H), 1.20-1.14 (m, 1H), 1.13-1.07 (m, 1H), 0.95 (dtd, J=8.9, 7.5, 5.2 Hz, 1H); HPLC purity: 100%; LCMS Calculated for C₂₂H₂₆N₂O₃S₂: 430.58; Observed: 431.2 [M+H]⁺.

### Example 60—Synthesis of 1-((4-(difluoromethyl)phenyl)sulfonyl)-2′,3′,5′,6′-tetrahydrospiro[indoline-3,4′-thiopyran] 1′,1′-dioxide (I-152)

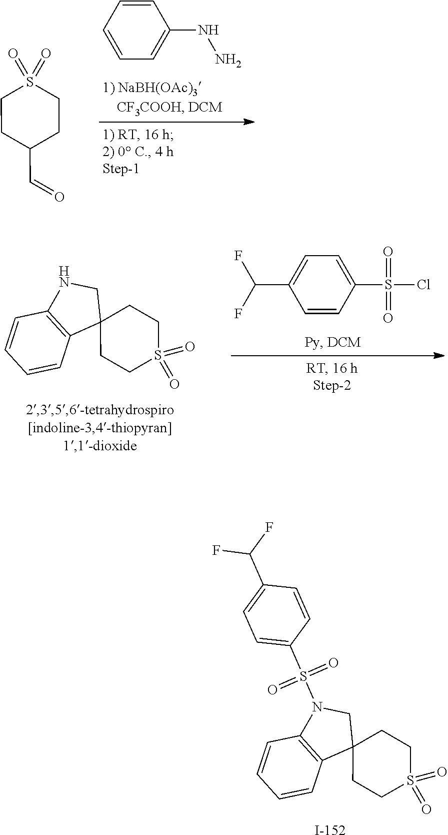 [SMILES: O=CC1CCS(=O)(=O)CC1 ; NNc1ccccc1 ; O=S1(=O)CCC2(CC1)CNc1ccccc12 ; O=S(=O)(Cl)c1ccc(C(F)F)cc1 ; O=S1(=O)CCC2(CC1)CN(S(=O)(=O)c1ccc(C(F)F)cc1)c1ccccc12 ; "I-152"]

### Step-1. Synthesis of 1,2-dihydrospiro[indole-3,4′-[1λ⁶]thiane]-1′,1′-dione

A solution of 1,1-dioxo-[1λ⁶-thiane-4-carbaldehyde] (1 g, 6.16 mmol) in dichloromethane (5 mL) was added dropwise to a mixture of phenyl hydrazine (0.9 g, 8.31 mmol) and trifluoroacetic acid (7 g, 61.6 mmol) in dichloromethane (20 mL). The mixture was stirred at 35° C. overnight. The reaction mixture was then cooled to 0° C., and sodium triacetoxyborohydride (3.89 g, 18.4 mmol) was added slowly to the solution, followed by stirring at RT for 4 hours. Afterwards, the mixture was washed with 6% NH₄OH aq. solution (25 mL) and brine (30 mL), dried over sodium sulfate, filtered, and evaporated to dryness to give 1,2-dihydrospiro[indole-3,4′-[1λ⁶]thiane]-1′,1′-dione (1.3 g, 5.47 mmol, 100% purity, 89% yield).

### Step-2. Synthesis of 1-[4-(difluoromethyl)benzenesulfonyl]-1,2-dihydrospiro[indole-3,4′-[1λ⁶]thiane]-1′,1′-dione

4-(difluoromethyl)benzene-1-sulfonyl chloride (0.251 g, 1.11 mmol) was added to an ice-cooled solution of 1,2-dihydrospiro[indole-3,4′-[1λ⁶]thiane]-1′,1′-dione (0.24 g, 1.01 mmol) and pyridine (0.399 g, 5.05 mmol) in dichloromethane (10 mL). The reaction mixture was allowed to warm to room temperature and stir until completion (overnight, NMR control). Afterwards, the reaction mixture was diluted with water (10 mL), and the organic layer was separated, dried over magnesium sulfate, filtered, and concentrated in vacuo. The residue was subjected to HPLC purification (deionized water/HPLC-grade acetonitrile) to give 1-[4-(difluoromethyl)benzenesulfonyl]-1,2-dihydrospiro[indole-3,4′-[1λ⁶]thiane]-1′,1′-dione (I-152). Yield: 192.3 mg, 42.2%; Appearance: Pink solid; ¹H NMR (400 MHz, DMSO-d₆) δ 8.02 (d, J=8.1 Hz, 2H), 7.77 (d, J=8.2 Hz, 2H), 7.52 (d, J=8.1 Hz, J H), 7.29 (t, J=7.8 Hz, 1H), 7.25-6.92 (m, 3H), 4.09 (s, 2H), 3.35 (t, J=13.3 Hz, 2H), 2.94 (d, J=14.1 Hz, 2H), 2.12 (t, J=13.5 Hz, 2H), 1.48 (d, J=14.2 Hz, 2H); HPLC purity: 100%; LCMS Calculated for C₁₉H₁₉F₂NO₄S₂: 427.48; Observed: 428.0 [M+H]⁺.

### Example 61

The following compounds were prepared according to the methods provided in the examples above, using routine modifications known and understood to a person of skill in the art.

| Compound No. | Structure | Analytical Data |
|---|---|---|
| I-153 | 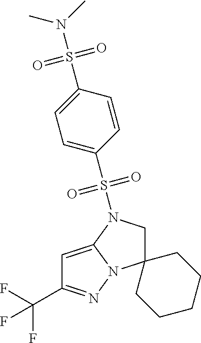 [SMILES: CN(C)S(=O)(=O)c1ccc(S(=O)(=O)N2CC3(CCCCC3)n3nc(C(F)(F)F)cc32)cc1] | Yield: 673.2 mg, 64.8%; Appearance: Beige solid; ¹H NMR (400 MHz, DMSO-d₆) δ 8.23- 8.15 (m, 2H), 8.05-7.94 (m, 2H), 6.45 (s, 1H), 4.29 (s, 2H), 2.62 (s, 6H), 1.69-1.55 (m, 4H), 1.47 (s, 1H), 1.31 (h, J = 12.4, 11.5 Hz, 5H); HPLC purity: 100%; LCMS Calculated for C₁₉H₂₃F₃N₄O₄S₂: 492.53; Observed: 493.0[M + H]⁺. |
| I-154 | 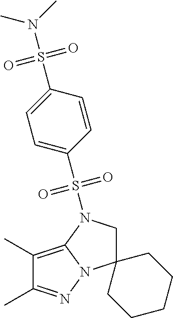 [SMILES: Cc1nn2c(c1C)N(S(=O)(=O)c1ccc(S(=O)(=O)N(C)C)cc1)CC21CCCCC1] | Yield: 702.7 mg, 73.7%; Appearance: Pink solid; ¹H NMR (400 MHz, DMSO-d₆) δ 8.07- 7.94 (m, 4H), 4.18 (s, 2H), 2.60 (s, 6H), 2.14- 2.06 (m, 6H), 1.60-1.50 (m, 2H), 1.49-1.36 (m, 3H), 1.31-1.06 (m, 3H), 0.89 (d, J = 12.9 Hz, 2H).; HPLC purity: 100%; LCMS Calculated for C₂₀H₂₈N₄O₄S₂: 452.59; Observed: 453.4[M + H]⁺. |
| I-155 | 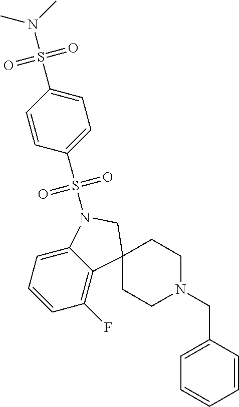 [SMILES: CN(C)S(=O)(=O)c1ccc(S(=O)(=O)N2CC3(CCN(Cc4ccccc4)CC3)c3c(F)cccc32)cc1] | Yield: 1170 mg, 53.4%; Appearance: Beige solid; ¹H NMR (600 MHz, DMSO-d₆) δ 8.05 (d, J = 8.4 Hz, 2H), 7.91 (d, J = 8.4 Hz, 2H), 7.36 (d, J = 8.1 Hz, 1H), 7.34-7.24 (m, 5H), 7.24-7.20 (m, 1H), 6.91-6.83 (m, 1H), 3.88 (s, 2H), 3.44 (s, 2H), 2.65-2.61 (m, 2H), 1.98-1.86 (m, 4H), 1.12-1.02 (m, 2H).; HPLC purity: 100%; LCMS Calculated for C₂₇H₃₀FN₃O₄S₂: 543.67; Observed: 544.2[M + H]⁺. |
| I-157 | 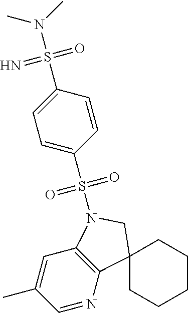 [SMILES: Cc1cnc2c(c1)N(S(=O)(=O)c1ccc(S(=N)(=O)N(C)C)cc1)CC21CCCCC1] | Yield: 157.2 mg, 22.6%; Appearance: Orange solid; ¹H NMR (400 MHz, DMSO-d₆) δ 8.08- 7.98 (m, 3H), 7.94 (d, J = 8.2 Hz, 2H), 7.65 (d, J = 1.8 Hz, 1H), 4.72 (s, 1H), 3.82 (s, 2H), 2.50 (s, 6H), 2.32 (s, 3H), 1.62-1.49 (m, 3H), 1.49-1.40 (m, 2H), 1.34-1.10 (m, 3H), 0.96 (m, 2H).; HPLC purity: 100%; LCMS Calculated for C₂₁H₂₈N₄O₃S₂: 448.6; Observed: 449.4[M + H]⁺. |
| I-158 | 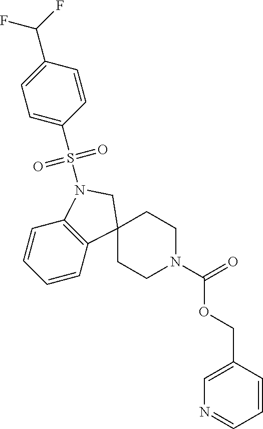 [SMILES: O=C(OCc1cccnc1)N1CCC2(CC1)CN(S(=O)(=O)c1ccc(C(F)F)cc1)c1ccccc12] | Yield: 37.5 mg, 29.9%; Appearance: Beige solid; ¹H NMR (400 MHz, DMSO-d₆) δ 8.54 (s, 1H), 8.51-8.40 (m, 1H), 7.95 (d, J = 8.0 Hz, 2H), 7.77-7.67 (m, 3H), 7.53 (d, J = 8.1 Hz, 1H), 7.33 (dd, J = 7.8, 4.8 Hz, 1H), 7.24-7.15 (m, 1H), 7.13-6.77 (m, 3H), 5.11 (s, 2H), 3.96 (d, J = 13.7 Hz, 2H), 3.89 (s, 2H), 3.00-2.78 (m, 2H), 1.71- 1.52 (m, 2H), 1.20 (d, J = 13.3 Hz, 2H).; HPLC purity: 100%; LCMS Calculated for C₂₆H₂₅F₂N₃O₄S: 513.56; Observed: 514.2[M + H]⁺. |
| I-159 | 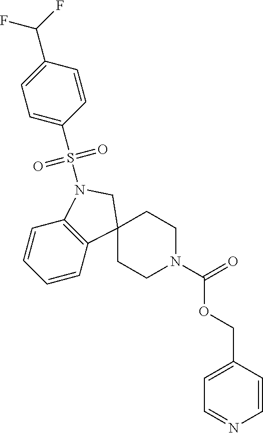 [SMILES: O=C(OCc1ccncc1)N1CCC2(CC1)CN(S(=O)(=O)c1ccc(C(F)F)cc1)c1ccccc12] | Yield: 38.9 mg, 31%; Appearance: White solid; ¹H NMR (400 MHz, DMSO-d₆) δ 8.51 (d, J = 5.9 Hz, 2H), 7.96 (d, J = 7.9 Hz, 2H), 7.73 (d, J = 8.1 Hz, 2H), 7.53 (d, J = 8.0 Hz, 1H), 7.27 (d, ) = 4.4 Hz, 2H), 7.24-7.16 (m, 1H), 7.18-6.74 (m, 3H), 5.11 (s, 2H), 4.08-3.93 (m, 2H), 3.91 (s, 2H), 2.99-2.83 (m, 2H), 1.71-1.59 (m, 2H), 1.23 (d, J = 13.3 Hz, 2H).; HPLC purity: 100%; LCMS Calculated for C₂₆H₂₅F₂N₃O₄S: 513.56; Observed: 514.0[M + H]⁺. |
| I-160 | 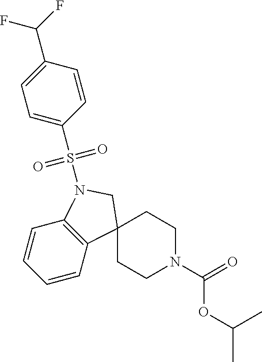 [SMILES: CC(C)OC(=O)N1CCC2(CC1)CN(S(=O)(=O)c1ccc(C(F)F)cc1)c1ccccc12] | Yield: 38.7 mg, 30.8%; Appearance: White solid; ¹H NMR (600 MHz, DMSO-d₆) δ 7.97 (d, J = 8.1 Hz, 2H), 7.76 (d, J = 8.0 Hz, 2H), 7.48 (d, J = 8.1 Hz, 1H), 7.25-7.07 (m, 3H), 7.07-6.96 (m, 1H), 4.77-4.67 (m, 1H), 3.90 (s, 2H), 3.87- 3.70 (m, 2H), 3.00-2.70 (m, 2H), 1.58-1.46 (m, 2H), 1.16 (d, J = 6.3 Hz, 6H), 1.07 (d, J = 13.2 Hz, 2H).; HPLC purity: 100%; LCMS Calculated C₂₃H₂₆F₂N₂O₄S: 464.53; Observed: 465.2[M + H]⁺. |
| I-161 | 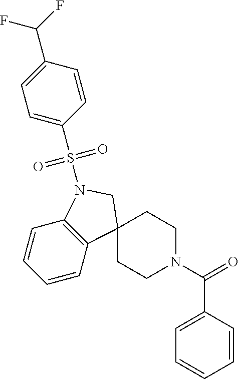 [SMILES: O=C(c1ccccc1)N1CCC2(CC1)CN(S(=O)(=O)c1ccc(C(F)F)cc1)c1ccccc12] | Yield: 32.8 mg, 26.1%; Appearance: Light brown solid; ¹H NMR (600 MHz, DMSO-d₆) δ 7.98 (d, J = 8.2 Hz, 2H), 7.75 (d, J = 8.1 Hz, 2H), 7.48 (d, J = 8.1 Hz, 1H), 7.46-7.38 (m, 5H), 7.30 (d, J = 7.5 Hz, 1H), 7.26-7.22 (m, 1H), 7.17- 6.96 (m, 2H), 4.35 (s, 1H), 3.96 (d, J = 18.7 Hz, 2H), 3.43 (s, 1H), 3.22-3.11 (m, 1H), 2.88 (s, 1H), 1.72-1.61 (m, 2H), 1.21-1.08 (m, 2H).; HPLC purity: 97.44%; LCMS Calculated for C₂₆H₂₄F₂N₂O₃S: 482.55; Observed: 483.2[M + H]⁺. |
| I-162 | 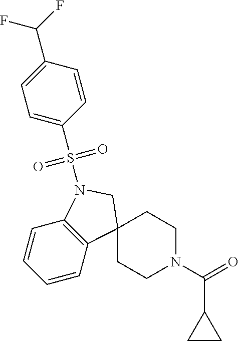 [SMILES: O=C(C1CC1)N1CCC2(CC1)CN(S(=O)(=O)c1ccc(C(F)F)cc1)c1ccccc12] | Yield: 33.3 mg, 26.7%; Appearance: Yellow solid; ¹H NMR (400 MHz, DMSO-d₆) δ 7.97 (d, J = 8.0 Hz, 2H), 7.73 (d, J = 8.1 Hz, 2H), 7.53 (d, J = 8.1 Hz, 1H), 7.24-7.15 (m, 1H), 7.14-6.79 (m, 3H), 4.31 (s, 1H), 4.14 (s, 1H), 4.06-3.84 (m, 2H), 3.84-3.72 (m, 1H), 3.33-3.21 (m, 1H), 3.19 (d, J = 5.1 Hz, 2H), 2.67 (s, 1H), 1.86 (s, 1H), 1.73-1.46 (m, 2H), 1.37-1.09 (m, 2H), 0.79 (s, 2H), 0.74-0.63 (m, 2H).; HPLC purity: 100%; LCMS Calculated for C₂₃H₂₄F₂N₂O₃S: 446.51; Observed: 447.2[M + H]⁺. |
| I-163 | 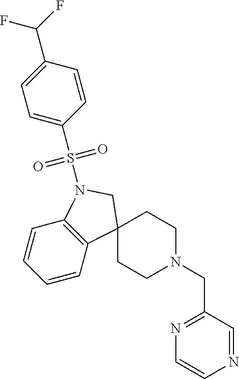 [SMILES: O=S(=O)(c1ccc(C(F)F)cc1)N1CC2(CCN(Cc3cnccn3)CC2)c2ccccc21] | Yield: 28.6 mg, 22.7%; Appearance: Yellow oil; ¹H NMR (600 MHz, DMSO-d₆) δ 8.67 (d, J = 1.5 Hz, 1H), 8.56 (dd, J = 2.6, 1.5 Hz, 1H), 8.52 (d, J = 2.6 Hz, 1H), 7.97 (d, J = 8.2 Hz, 2H), 7.74 (d, J = 8.2 Hz, 2H), 7.48 (d, J = 8.0 Hz, 1H), 7.27- 6.95 (m, 4H), 3.82 (s, 2H), 3.66 (s, 2H), 2.69 (d, J = 11.7 Hz, 2H), 2.18-2.08 (m, 2H), 1.74-1.64 (m, 2H), 1.09 (d, J = 12.5 Hz, 2H).; HPLC purity: 97.4%; LCMS Calculated for C₂₄H₂₄F₂N₄O₂S: 470.54; Observed: 471.2[M + H]⁺. |
| I-164 | 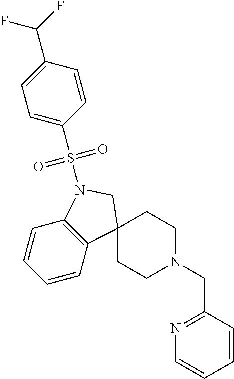 [SMILES: O=S(=O)(c1ccc(C(F)F)cc1)N1CC2(CCN(Cc3ccccn3)CC2)c2ccccc21] | Yield: 48.8 mg, 38.9%; Appearance: Light brown solid; ¹H NMR (600 MHz, DMSO-d₆) δ 8.52-8.41 (m, 1H), 7.97 (d, J = 8.2 Hz, 2H), 7.77- 7.70 (m, 3H), 7.48 (d, J = 8.1 Hz, 1H), 7.42 (d, J = 7.8 Hz, 1H), 7.25-6.93 (m, 5H), 3.82 (s, 2H), 3.59 (s, 2H), 2.68 (d, J = 12.4 Hz, 2H), 2.09 (t, J = 12.3 Hz, 2H), 1.75-1.63 (m, 2H), 1.14-1.04 (m, 2H).; HPLC purity: 100%; LCMS Calculated for C₂₅H₂₅F₂N₃O₂S: 469.55; Observed: 470.0[M + H]⁺. |
| I-165 | 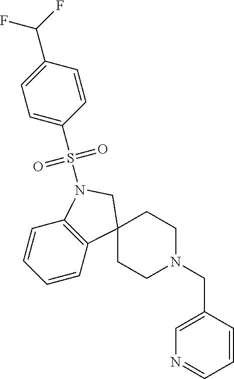 [SMILES: O=S(=O)(c1ccc(C(F)F)cc1)N1CC2(CCN(Cc3cccnc3)CC2)c2ccccc21] | Yield: 30.9 mg, 24.6%; Appearance: Yellow solid; ¹H NMR (600 MHz, DMSO-d₆) δ 8.48 (d, J = 2.1 Hz, 1H), 8.45 (dd, J = 4.8, 1.6 Hz, 1H), 7.96 (d, J = 8.2 Hz, 2H), 7.74 (d, J = 8.1 Hz, 2H), 7.72-7.59 (m, 1H), 7.47 (d, J = 8.0 Hz, 1H), 7.34 (dd, J = 7.8, 4.8 Hz, 1H), 7.27-7.19 (m, 2H), 7.19-6.90 (m, 2H), 3.81 (s, 2H), 3.50 (s, 2H), 2.64 (d, J = 11.7 Hz, 2H), 2.08-1.96 (m, 2H), 1.72-1.59 (m, 2H), 1.10 (d, J = 13.0 Hz, 2H).; HPLC purity: 100%; LCMS Calculated for C₂₅H₂₅F₂N₃O₂S: 469.55; Observed: 470.0[M + H]⁺. |
| I-166 | 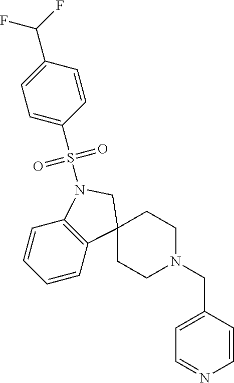 [SMILES: O=S(=O)(c1ccc(C(F)F)cc1)N1CC2(CCN(Cc3ccncc3)CC2)c2ccccc21] | Yield: 35.7 mg, 28.4%; Appearance: Yellow oil; ¹H NMR (600 MHz, DMSO-d₆) δ 8.53-8.45 (m, 2H), 7.96 (d, J = 8.0 Hz, 2H), 7.74 (d, J = 8.0 Hz, 2H), 7.48 (d, J = 7.9 Hz, 1H), 7.33-7.29 (m, 2H), 7.25-7.18 (m, 2H), 7.17-6.95 (m, 2H), 3.81 (s, 2H), 3.51 (s, 2H), 2.64 (d, J = 11.8 Hz, 2H), 2.05 (t, J = 12.0 Hz, 2H), 1.75-1.63 (m, 2H), 1.10 (d, J = 12.9 Hz, 2H).; HPLC purity: 100%; LCMS Calculated for C₂₅H₂₅F₂N₃O₂S: 469.55; Observed: 470.0[M + H]⁺. |
| I-167 | 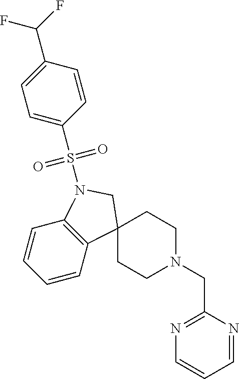 [SMILES: O=S(=O)(c1ccc(C(F)F)cc1)N1CC2(CCN(Cc3ncccn3)CC2)c2ccccc21] | Yield: mg, %; Appearance: Yellow solid; ¹H NMR (400 MHz, DMSO-d₆) δ 8.78 (d, J = 4.9 Hz, 2H), 7.98 (d, J = 8.0 Hz, 2H), 7.76 (d, J = 8.0 Hz, 2H), 7.49 (d, J = 8.0 Hz, 1H), 7.43-7.35 (m, 1H), 7.26-6.92 (m, 4H), 3.82 (s, 2H), 3.71 (s, 2H), 2.78 (d, J = 11.8 Hz, 2H), 2.18 (t, J = 12.0 Hz, 2H), 1.75-1.61 (m, 2H), 1.09 (d, J = 12.9 Hz, 2H).; HPLC purity: 96.39%; LCMS Calculated for C₂₄H₂₄F₂N₄O₂S: 470.54; Observed: 471.2[M + H]⁺. |
| I-168 | 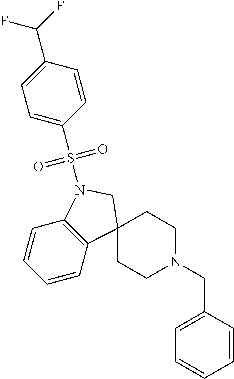 [SMILES: O=S(=O)(c1ccc(C(F)F)cc1)N1CC2(CCN(Cc3ccccc3)CC2)c2ccccc21] | Yield: 26.8 mg, 21.3%; Appearance: Yellow solid; ¹H NMR (600 MHz, DMSO-d₆) δ 7.96 (d, J = 8.1 Hz, 2H), 7.74 (d, J = 8.1 Hz, 2H), 7.47 (d, J = 8.0 Hz, 1H), 7.33-7.27 (m, 4H), 7.25-7.19 (m, 3H), 7.16-6.96 (m, 2H), 3.80 (s, 2H), 3.46 (s, 2H), 2.65 (d, J = 12.4 Hz, 2H), 2.03-1.94 (m, 2H), 1.71-1.61 (m, 2H), 1.09 (d, J = 12.9 Hz, 2H).; HPLC purity: 100%; LCMS Calculated for C₂₆H₂₆F₂N₂O₂S: 468.56; Observed: 469.0[M + H]⁺. |
| I-169 | 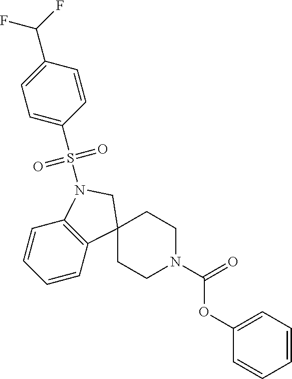 [SMILES: O=C(Oc1ccccc1)N1CCC2(CC1)CN(S(=O)(=O)c1ccc(C(F)F)cc1)c1ccccc12] | Yield: 156.2 mg, 75.1%; Appearance: Yellow solid; ¹H NMR (500 MHz, DMSO-d₆) δ 7.99 (d, J = 8.0 Hz, 2H), 7.77 (d, J = 8.0 Hz, 2H), 7.50 (d, J = 8.1 Hz, 1H), 7.40-7.32 (m, 2H), 7.29-7.23 (m, 2H), 7.22-6.96 (m, 5H), 4.07-3.99 (m, 1H), 3.97 (s, 2H), 3.94-3.83 (m, 1H), 3.20-3.08 (m, 1H), 3.05-2.93 (m, 1H), 1.81-1.61 (m, 2H), 1.16 (d, J = 13.0 Hz, 2H).; HPLC purity: 98.27%; LCMS Calculated for C₂₆H₂₄F₂N₂O₄S: 498.54; Observed: 499.2[M + H]⁺. |
| I-170 | 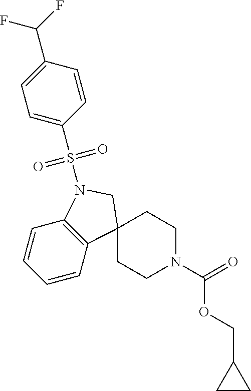 [SMILES: O=C(OCC1CC1)N1CCC2(CC1)CN(S(=O)(=O)c1ccc(C(F)F)cc1)c1ccccc12] | Yield: 58.9 mg, 46.9%; Appearance: White solid; ¹H NMR (400 MHz, DMSO-d₆) δ 7.95 (d, J = 8.0 Hz, 2H), 7.73 (d, J = 8.0 Hz, 2H), 7.53 (d, J = 8.1 Hz, 1H), 7.27-7.13 (m, 1H), 7.12-6.79 (m, 3H), 3.95 (d, J = 13.7 Hz, 2H), 3.88 (s, 2H), 3.83 (d, J = 7.0 Hz, 2H), 2.98-2.78 (m, 2H), 1.68- 1.54 (m, 2H), 1.29-1.16 (m, 2H), 1.15-1.02 (m, 1H), 0.59-0.45 (m, 2H), 0.32-0.22 (m, 2H).; HPLC purity: 100%; LCMS Calculated for C₂₄H₂₆F₂N₂O₄S: 476.54; Observed: 477.2[M + H]⁺. |
| I-171 | 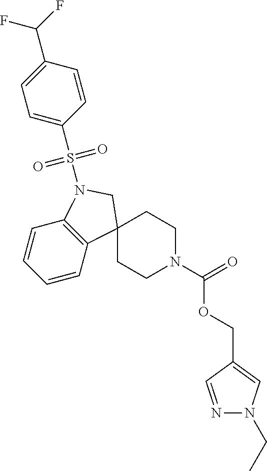 [SMILES: CCn1cc(COC(=O)N2CCC3(CC2)CN(S(=O)(=O)c2ccc(C(F)F)cc2)c2ccccc23)cn1] | Yield: 17.5 mg, 16.6%; Appearance: White solid; ¹H NMR (600 MHz, DMSO-d₆) δ 7.96 (d, J = 8.1 Hz, 2H), 7.79-7.70 (m, 3H), 7.48 (d, J = 8.2 Hz, 1H), 7.42 (s, 1H), 7.27-7.21 (m, 1H), 7.20-6.94 (m, 3H), 4.90 (s, 2H), 4.08 (q, J = 7.3 Hz, 2H), 3.90 (s, 2H), 3.81 (d, J = 34.4 Hz, 2H), 2.98-2.74 (m, 2H), 1.57-1.46 (m, 2H), 1.33 (t, J = 7.3 Hz, 3H), 1.05 (d, J = 13.1 Hz, 2H).; HPLC purity: 100%; LCMS Calculated for C₂₆H₂₈F₂N₄O₄S: 530.59; Observed: 531.2[M + H]⁺. |
| I-172 | 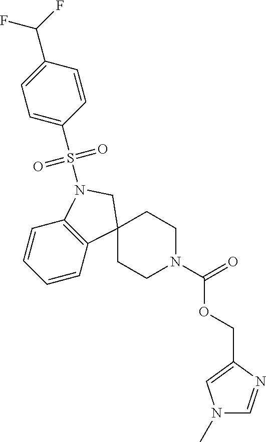 [SMILES: Cn1cnc(COC(=O)N2CCC3(CC2)CN(S(=O)(=O)c2ccc(C(F)F)cc2)c2ccccc23)c1] | Yield: 72.6 mg, 62.6%; Appearance: Yellow solid; ¹H NMR (400 MHz, DMSO-d₆) δ 7.98 (d, J = 8.1 Hz, 2H), 7.77 (d, J = 8.1 Hz, 2H), 7.53 (d, J = 1.3 Hz, 1H), 7.50 (d, J = 8.1 Hz, 1H), 7.32- 6.95 (m, 5H), 4.87 (s, 2H), 3.92 (s, 2H), 3.90- 3.72 (m, 2H), 3.00-2.78 (m, 2H), 1.62-1.49 (m, 2H), 1.07 (d, J = 13.2 Hz, 2H).; HPLC purity: 100%; LCMS Calculated for C₂₅H₂₆F₂N₄O₄S: 516.56; Observed: 517.0[M + H]⁺. |
| I-173 | 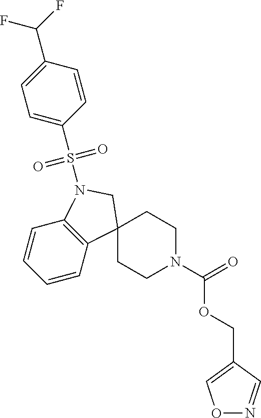 [SMILES: O=C(OCc1cnoc1)N1CCC2(CC1)CN(S(=O)(=O)c1ccc(C(F)F)cc1)c1ccccc12] | Yield: 41.6 mg, 20.2%; Appearance: Yellow oil; ¹H NMR (400 MHz, DMSO-d₆) δ 9.01 (s, 1H), 8.66 (s, 1H), 7.99 (d, J = 8.0 Hz, 2H), 7.77 (d, J = 8.0 Hz, 2H), 7.50 (d, J = 8.1 Hz, 1H), 7.31-6.94 (m, 4H), 4.98 (s, 2H), 3.92 (s, 2H), 3.89-3.76 (m, 2H), 3.06-2.76 (m, 2H), 1.65-1.51 (m, 2H), 1.08 (d, J = 13.3 Hz, 2H).; HPLC purity: 100%; LCMS Calculated for C₂₄H₂₃F₂N₃O₅S: 504.52; Observed: 505.2[M + H]⁺. |
| I-174 | 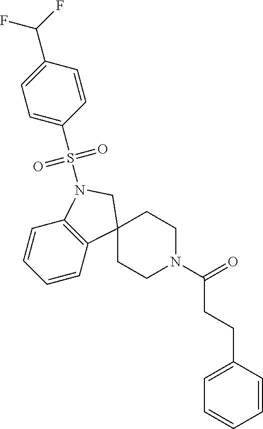 [SMILES: O=C(CCc1ccccc1)N1CCC2(CC1)CN(S(=O)(=O)c1ccc(C(F)F)cc1)c1ccccc12] | Yield: 57.6 mg, 54.9%; Appearance: White solid; ¹H NMR (400 MHz, DMSO-d₆) δ 7.95 (d, J = 8.1 Hz, 2H), 7.72 (d, J = 8.1 Hz, 2H), 7.52 (d, J = 8.1 Hz, 1H), 7.26-7.12 (m, 6H), 7.10-6.80 (m, 3H), 4.36 (d, J = 13.7 Hz, 1H), 3.93-3.78 (m, 2H), 3.73 (d, J = 13.9 Hz, 1H), 3.09 (t, J = 13.4 Hz, 1H), 2.85 (t, J = 7.6 Hz, 2H), 2.70-2.55 (m, 3H), 1.47 (q, J = 13.5 Hz, 2H), 1.17 (t, J = 11.8 Hz, 2H).; HPLC purity: 100%; LCMS Calculated for C₂₈H₂₈F₂N₂O₃S: 510.6; Observed: 511.2[M + H]⁺. |
| I-175 | 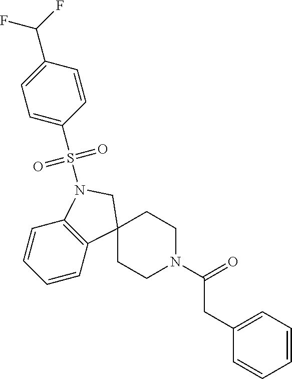 [SMILES: O=C(Cc1ccccc1)N1CCC2(CC1)CN(S(=O)(=O)c1ccc(C(F)F)cc1)c1ccccc12] | Yield: 24.1 mg, 22.8%; Appearance: Yellow solid; ¹H NMR (600 MHz, DMSO-d₆) δ 7.97 (d, J = 8.2 Hz, 2H), 7.75 (d, J = 8.0 Hz, 2H), 7.47 (d, J = 8.1 Hz, 1H), 7.29 (dd, J = 8.4, 6.9 Hz, 2H), 7.27-7.18 (m, 4H), 7.11-6.93 (m, 3H), 4.26 (d, J = 13.7 Hz, 1H), 3.95-3.86 (m, 2H), 3.82 (d, J = 14.2 Hz, 1H), 3.77-3.60 (m, 2H), 3.07 (t, J = 13.6 Hz, 1H), 2.66 (t, J = 13.1 Hz, 1H), 1.48- 1.34 (m, 2H), 1.12-0.95 (m, 2H).; HPLC purity: 100%; LCMS Calculated for C₂₇H₂₆F₂N₂O₃S; 496.57; Observed: 497.0[M + H]⁺. |
| I-176 | 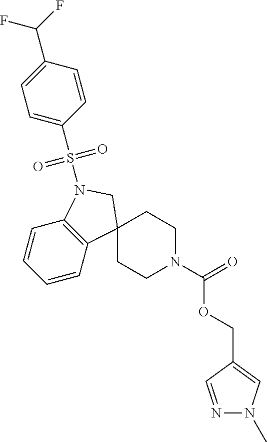 [SMILES: Cn1cc(COC(=O)N2CCC3(CC2)CN(S(=O)(=O)c2ccc(C(F)F)cc2)c2ccccc23)cn1] | Yield: 16 mg, 15.2%; Appearance: Beige solid; ¹H NMR (600 MHz, DMSO-d₆) δ 7.96 (d, J = 8.2 Hz, 2H), 7.75 (d, J = 8.1 Hz, 2H), 7.70 (s, 1H), 7.48 (d, J = 8.1 Hz, 1H), 7.41 (s, 1H), 7.28-7.20 (m, 1H), 7.20-6.99 (m, 3H), 4.89 (d, J = 9.1 Hz, 2H), 3.89 (s, 2H), 3.88-3.79 (m, 2H), 3.79 (s, 3H), 2.98-2.78 (m, 2H), 1.58-1.44 (m, 2H), 1.05 (d, J = 12.8 Hz, 2H).; HPLC purity: 100%; LCMS Calculated for C₂₅H₂₆F₂N₄O₄S: 516.56; Observed: 517.0[M + H]⁺. |
| I-177 | 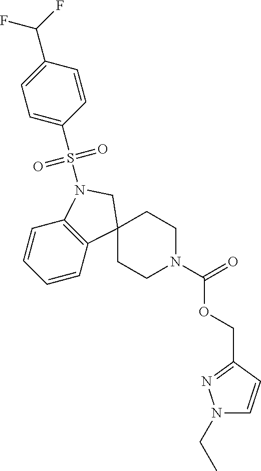 [SMILES: CCn1ccc(COC(=O)N2CCC3(CC2)CN(S(=O)(=O)c2ccc(C(F)F)cc2)c2ccccc23)n1] | Yield: 92.4 mg, 41.1%; Appearance: Beige solid; ¹H NMR (400 MHz, DMSO-d₆) δ 7.99 (d, J = 8.1 Hz, 2H), 7.77 (d, J = 8.0 Hz, 2H), 7.69 (d, J = 2.2 Hz, 1H), 7.50 (d, J = 8.1 Hz, 1H), 7.31- 6.92 (m, 4H), 6.22 (d, J = 2.2 Hz, 1H), 4.97 (s, 2H), 4.09 (q, J = 7.3 Hz, 2H), 3.92 (s, 2H), 3.89 (s, 0H), 3.83 (s, 2H), 1.63-1.51 (m, 2H), 1.35 (t, J = 7.3 Hz, 3H), 1.07 (d, J = 13.2 Hz, 2H)., HPLC purity: 97.29%; LCMS Calculated for C₂₆H₂₈F₂N₄O₄S: 530.59; Observed: 531.2[M + H]⁺. |
| I-178 | 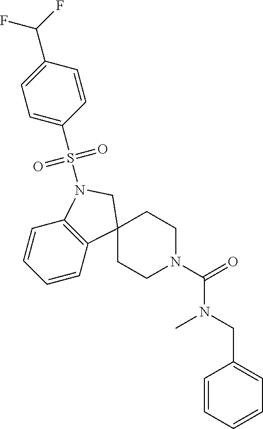 [SMILES: CN(Cc1ccccc1)C(=O)N1CCC2(CC1)CN(S(=O)(=O)c1ccc(C(F)F)cc1)c1ccccc12] | Yield: 8.5 mg, 6.16%; Appearance: Yellow solid; ¹H NMR (600 MHz, DMSO-d₆) δ 7.98 (d, J = 8.5 Hz, 2H), 7.75 (d, J = 8.1 Hz, 2H), 7.48 (d, J = 8.1 Hz, 1H), 7.36-7.27 (m, 2H), 7.27 - 6.94 (m, 7H), 4.30 (s, 2H), 3.91 (s, 2H), 3.44 (d, J = 13.5 Hz, 2H), 3.15 (dd, J = 5.2, 2.1 Hz, 1H), 2.83 (t, J = 13.4 Hz, 2H), 2.66 (s, 3H), 1.72-1.55 (m, 2H), 1.08 (d, J = 13.0 Hz, 2H).; HPLC purity: 100%; LCMS Calculated for C₂₈H₂₉F₂N₃O₃S: 525.61; Observed: 526.4[M + H]⁺. |
| I-179 | 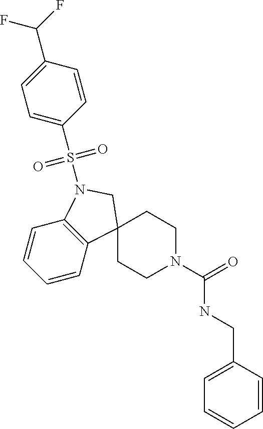 [SMILES: O=C(NCc1ccccc1)N1CCC2(CC1)CN(S(=O)(=O)c1ccc(C(F)F)cc1)c1ccccc12] | Yield: 208.6 mg, 48.8%; Appearance: White solid; ¹H NMR (400 MHz, DMSO-d₆) δ 8.00 (d, J = 8.1 Hz, 2H), 7.77 (d, J = 8.1 Hz, 2H), 7.51 (d, J = 8.0 Hz, 1H), 7.32-7.17 (m, 7H), 7.12-7.02 (m, 2H), 4.23 (d, J = 5.7 Hz, 2H), 3.94 (s, 2H), 3.89 (d, J = 13.7 Hz, 2H), 2.80 (t, J = 12.9 Hz, 2H), 1.53 (td, J = 13.0, 4.3 Hz, 2H), 1.07 (d, J = 13.0 Hz, 2H); HPLC purity: 100%; LCMS Calculated for C₂₇H₂₇F₂N₃O₃S: 511.59; Observed: 512.2[M + H]⁺. |
| I-180 | 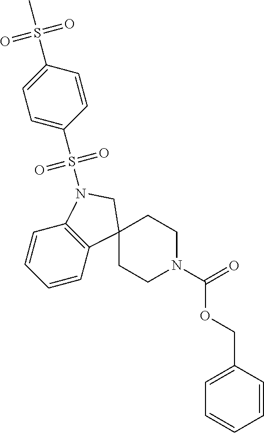 [SMILES: CS(=O)(=O)c1ccc(S(=O)(=O)N2CC3(CCN(C(=O)OCc4ccccc4)CC3)c3ccccc32)cc1] | Yield: 50.1 mg, 31.6%; Appearance: Yellow solid; ¹H NMR (600 MHz, DMSO-d₆) δ 8.11 (s, 4H), 7.49 (d, J = 8.0 Hz, 1H), 7.39-7.29 (m, 5H), 7.27-7.20 (m, 2H), 7.08-6.99 (m, 1H), 5.07 (s, 2H), 3.95 (s, 2H), 3.89 (d, J = 13.6 Hz, 2H), 3.26 (s, 3H), 3.07-2.79 (m, 2H), 2.05 (s, 2H), 1.64- 1.51 (m, 2H), 1.15 (d, J = 13.2 Hz, 2H).; HPLC purity: 98%; LCMS Calculated for C₂₇H₂₈N₂O₆S₂: 540.65; Observed: 541.2[M + H]⁺. |
| I-181 | 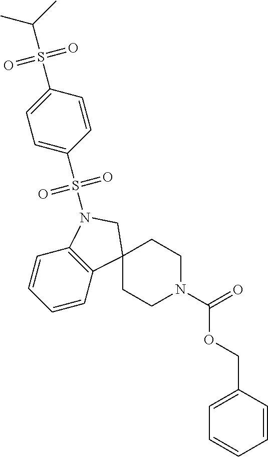 [SMILES: CC(C)S(=O)(=O)c1ccc(S(=O)(=O)N2CC3(CCN(C(=O)OCc4ccccc4)CC3)c3ccccc32)cc1] | Yield: 63.8 mg, 18.9%; Appearance: White solid; ¹H NMR (400 MHz, DMSO-d₆) δ 8.07 (q, J = 8.3 Hz, 4H), 7.53 (d, J = 8.1 Hz, 1H), 7.42- 7.31 (m, 5H), 7.28 (t, J = 7.8 Hz, 1H), 7.23 (d, J = 7.5 Hz, 1H), 7.07 (t, J = 7.5 Hz, 1H), 5.06 (s, 2H), 3.96 (s, 2H), 3.85 (s, 2H), 3.51 (p, J = 6.6 Hz, 1H), 2.94 (s, 2H), 1.56 (s, 2H), 1.08 (d, J = 6.8 Hz, 6H), 1.04 (s, 2H); HPLC purity: 100%; LCMS Calculated for C₂₉H₃₂N₂O₆S₂: 569.7; Observed: 570.0[M + H]⁺. |
| I-182 | 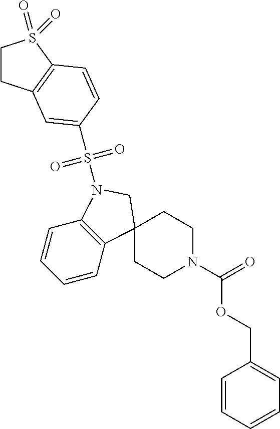 [SMILES: O=C(OCc1ccccc1)N1CCC2(CC1)CN(S(=O)(=O)c1ccc3c(c1)CCS3(=O)=O)c1ccccc12] | Yield: 332.3 mg, 30.5%; Appearance: White solid; ¹H NMR (600 MHz, DMSO-d₆) δ 8.12 (s, 1H), 7.95-7.90 (m, 2H), 7.47 (d, J = 8.0 Hz, 1H), 7.36 (h, J = 5.9 Hz, 4H), 7.31 (td, J = 6.5, 6.0, 2.5 Hz, 1H), 7.25-7.20 (m, 2H), 7.02 (t, J = 7.5 Hz, 1H), 5.07 (s, 2H), 3.95 (s, 2H), 3.91 (d, J = 14.1 Hz, 2H), 3.64 (t, J = 6.9 Hz, 2H), 3.39 (t, J = 6.9 Hz, 2H), 2.97 (d, J = 42.7 Hz, 2H), 1.60 (td, J = 13.2, 4.5 Hz, 2H), 1.19 (d, J = 13.3 Hz, 2H); HPLC purity: 100%; LCMS Calculated for C₂₈H₂₈N₂O₆S₂: 553.66; Observed: 554.0[M + H]⁺. |
| I-183 | 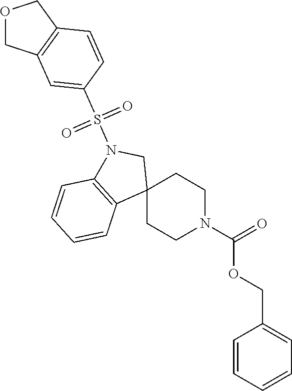 [SMILES: O=C(OCc1ccccc1)N1CCC2(CC1)CN(S(=O)(=O)c1ccc3c(c1)COC3)c1ccccc12] | Yield: 89.1 mg, 56.7%; Appearance: Yellow solid; ¹H NMR (400 MHz, DMSO-d₆) δ 7.79 (s, 1H), 7.72 (d, J = 7.9 Hz, 1H), 7.51 (d, J = 8.1 Hz, 1H), 7.47-7.39 (m, 1H), 7.38-7.31 (m, 4H), 7.31-7.23 (m, 1H), 7.25-7.14 (m, 1H), 7.08 (d, J = 7.4 Hz, 1H), 7.02-6.92 (m, 1H), 5.07 (s, 2H), 5.03 (s, 4H), 3.99 (d, J = 13.6 Hz, 2H), 3.87 (s, 2H), 3.01-2.80 (m, 2H), 1.72-1.54 (m, 2H), 1.25 (d, J = 13.2 Hz, 2H).; HPLC purity: 99%; LCMS Calculated for C₂₈H₂₈N₂O₅S: 504.6; Observed: 505.2[M + H]⁺. |
| I-184 | 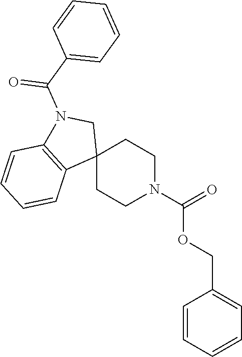 [SMILES: O=C(OCc1ccccc1)N1CCC2(CC1)CN(C(=O)c1ccccc1)c1ccccc12] | Yield: 41.8 mg, 28.6%; Appearance: Beige solid; ¹H NMR (600 MHz, DMSO-d₆) δ 7.61- 7.54 (m, 2H), 7.54-7.50 (m, 1H), 7.51-7.46 (m, 2H), 7.39-7.33 (m, 4H), 7.31 (d, J = 7.3 Hz, 2H), 7.24-6.94 (m, 2H), 5.07 (s, 2H), 3.96 (s, 4H), 3.01-2.72 (m, 2H), 1.79-1.69 (m, 2H), 1.65 (d, J = 13.3 Hz, 2H).; HPLC purity: 100%; LCMS Calculated for C₂₇H₂₆N₂O₃: 426.52; Observed: 427.2[M + H]⁺. |
| I-185 | 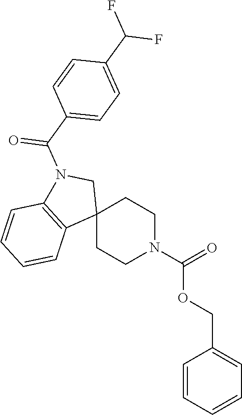 [SMILES: O=C(OCc1ccccc1)N1CCC2(CC1)CN(C(=O)c1ccc(C(F)F)cc1)c1ccccc12] | Yield: 72.3 mg, 46%; Appearance: Yellow solid; ¹H NMR (600 MHz, DMSO-d₆) δ 7.73 (s, 2H), 7.68 (d, J = 7.7 Hz, 2H), 7.38-7.28 (m, 6H), 7.25-6.98 (m, 3H), 5.07 (s, 2H), 4.06-3.85 (m, 4H), 3.02-2.71 (m, 2H), 1.80-1.69 (m, 2H), 1.69-1.60 (m, 2H).; HPLC purity: 100%; LCMS Calculated for C₂₈H₂₆F₂N₂O₃: 476.52; Observed: 477.2[M + H]⁺. |
| I-186 | 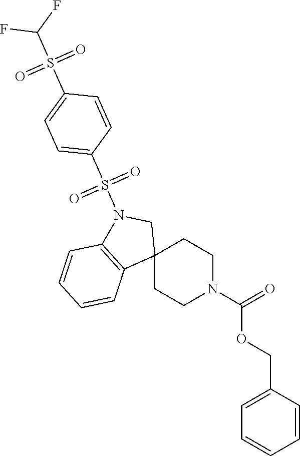 [SMILES: O=C(OCc1ccccc1)N1CCC2(CC1)CN(S(=O)(=O)c1ccc(S(=O)(=O)C(F)F)cc1)c1ccccc12] | Yield: 87.3 mg, 55.6%; Appearance: Orange solid; ¹H NMR (600 MHz, DMSO-d₆) δ 8.21- 8.13 (m, 4H), 7.51 (d, J = 8.1 Hz, 1H), 7.48-7.18 (m, 8H), 7.09-7.01 (m, 1H), 5.05 (s, 2H), 3.95 (s, 2H), 3.84 (d, J = 13.9 Hz, 2H), 3.01-2.79 (m, 2H), 1.60-1.50 (m, 2H), 0.98 (s, 2H).; HPLC purity: 100%; LCMS Calculated for C₂₇H₂₆F₂N₂O₆S₂: 577.63; Observed: 578.8[M + H]⁺. |
| I-187 | 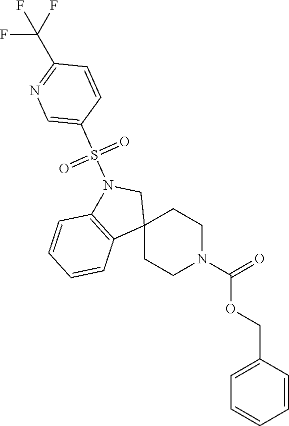 [SMILES: O=C(OCc1ccccc1)N1CCC2(CC1)CN(S(=O)(=O)c1ccc(C(F)(F)F)nc1)c1ccccc12] | Yield: 83.7 mg, 53.3%; Appearance: Orange solid; ¹H NMR (600 MHz, DMSO-d₆) δ 9.24 (d, J = 2.1 Hz, 1H), 8.55 (dd, J = 8.3, 2.2 Hz, 1H), 8.13 (d, J = 8.3 Hz, 1H), 7.52 (d, J = 8.0 Hz, 1H), 7.40-7.33 (m, 4H), 7.32-7.28 (m, 1H), 7.27- 7.23 (m, 2H), 7.10-6.99 (m, 1H), 5.07 (s, 2H), 3.99 (s, 2H), 3.91 (d, J = 13.6 Hz, 2H), 3.10-2.82 (m, 2H), 1.70-1.55 (m, 2H), 1.24 (d, J = 13.2 Hz, 2H).; HPLC purity: 100%; LCMS Calculated for C₂₆H₂₄F₃N₃O₄S: 531.55; Observed: 532.0[M + H]⁺. |
| I-188 | 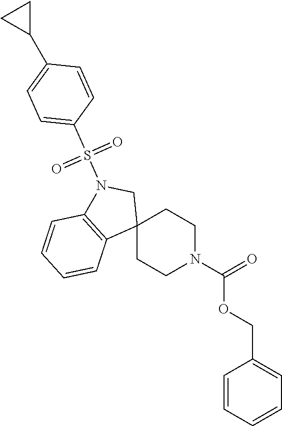 [SMILES: O=C(OCc1ccccc1)N1CCC2(CC1)CN(S(=O)(=O)c1ccc(C3CC3)cc1)c1ccccc12] | Yield: 77.9 mg, 49.6%; Appearance: Orange solid; ¹H NMR (400 MHz, DMSO-d₆) δ 7.65 (d, J = 8.0 Hz, 2H), 7.51 (d, J = 8.1 Hz, 1H), 7.40- 7.24 (m, 5H), 7.24-7.10 (m, 3H), 7.06 (d, J = 7.5 Hz, 1H), 7.01-6.91 (m, 1H), 5.07 (s, 2H), 3.97 (d, J = 13.6 Hz, 2H), 3.81 (s, 2H), 2.98-2.80 (m, 2H), 1.99-1.86 (m, 1H), 1.66-1.51 (m, 2H), 1.19 (d, J = 13.4 Hz, 2H), 1.12-0.97 (m, 2H), 0.80-0.65 (m, 2H).; HPLC purity: 99%; LCMS Calculated for C₂₉H₃₀N₂O₄S: 502.63; Observed: 503.2[M + H]⁺. |
| I-189 | 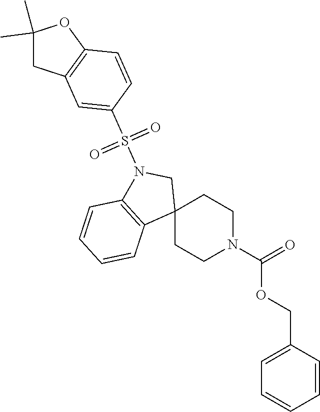 [SMILES: CC1(C)Cc2cc(S(=O)(=O)N3CC4(CCN(C(=O)OCc5ccccc5)CC4)c4ccccc43)ccc2O1] | Yield: 90.4 mg, 57.5%; Appearance: Yellow solid; ¹H NMR (400 MHz, DMSO-d₆) δ 7.67- 7.59 (m, 1H), 7.55-7.47 (m, 2H), 7.38-7.24 (m, 5H), 7.23-7.14 (m, 1H), 7.06 (d, J = 7.5 Hz, 1H), 7.01-6.88 (m, 1H), 6.70 (d, J = 8.6 Hz, 1H), 5.06 (s, 2H), 3.99 (d, J = 13.6 Hz, 2H), 3.81 (s, 2H), 2.98-2.85 (m, 2H), 1.71-1.53 (m, 2H), 1.44 (s, 6H), 1.22 (d, J = 13.4 Hz, 2H).; HPLC purity: 100%; LCMS Calculated for C₃₀H₃₂N₂O₅S: 532.66; Observed: 533.2[M + H]⁺. |
| I-190 | 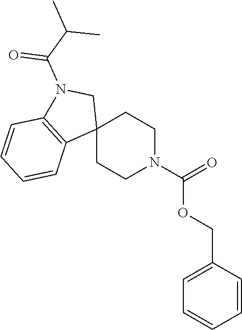 [SMILES: CC(C)C(=O)N1CC2(CCN(C(=O)OCc3ccccc3)CC2)c2ccccc21] | Yield: 88.9 mg, 56.6%; Appearance: Light brown solid; ¹H NMR (600 MHz, DMSO-d₆) δ 8.08 (d, J = 8.1 Hz, 1H), 7.40-7.35 (m, 4H), 7.35- 7.28 (m, 1H), 7.25 (d, J = 7.5 Hz, 1H), 7.18- 7.11 (m, 1H), 7.03-6.93 (m, 1H), 5.10 (s, 2H), 4.08 (s, 2H), 4.01 (d, J = 13.5 Hz, 2H), 3.13-2.97 (m, 2H), 2.97-2.87 (m, 1H), 1.78-1.67 (m, 2H), 1.61 (d, J = 13.3 Hz, 2H), 1.09 (d, J = 6.7 Hz, 6H).; HPLC purity: 100%; LCMS Calculated for C₂₄H₂₈N₂O₃: 392.5; Observed: 393.2[M + H]⁺. |
| I-191 | 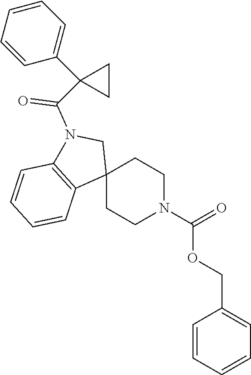 [SMILES: O=C(OCc1ccccc1)N1CCC2(CC1)CN(C(=O)C1(c3ccccc3)CC1)c1ccccc12] | Yield: 14.2 mg, 9.05%; Appearance: Yellow solid; ¹H NMR (600 MHz, DMSO-d₆) δ 8.02 (s, 1H), 7.40-7.27 (m, 7H), 7.27-7.12 (m, 5H), 7.06-6.94 (m, 1H), 5.05 (s, 2H), 3.83 (d, J = 13.6 Hz, 2H), 3.78 (s, 2H), 2.64-2.50 (m, 2H), 1.67- 1.54 (m, 2H), 1.44 (s, 2H), 1.33 (d, J = 13.3 Hz, 2H), 1.26 (s, 2H).; HPLC purity: 100%, LCMS Calculated for C₃₀H₃₀N₂O₃: 466.58; Observed: 467.1[M + H]⁺. |
| I-192 | 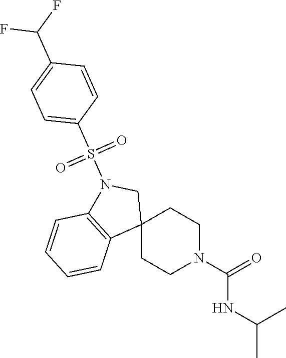 [SMILES: CC(C)NC(=O)N1CCC2(CC1)CN(S(=O)(=O)c1ccc(C(F)F)cc1)c1ccccc12] | Yield: 43.2 mg, 34.4%; Appearance: Yellow solid; ¹H NMR (400 MHz, DMSO-d₆) δ 7.95 (d, J = 8.1 Hz, 2H), 7.73 (d, J = 8.0 Hz, 2H), 7.53 (d, J = 8.1 Hz, 1H), 7.24-7.17 (m, 1H), 7.12-6.81 (m, 3H), 5.95 (d, J = 7.7 Hz, 1H), 3.89 (d, J = 13.2 Hz, 2H), 3.85 (s, 2H), 3.81-3.71 (m, 1H), 2.72 (t, J = 13.3 Hz, 2H), 1.64-1.49 (m, 2H), 1.17 (d, J = 13.3 Hz, 2H), 1.12-0.99 (m, 6H); HPLC purity: 100%; LCMS Calculated for C₂₃H₂₇F₂N₃O₃S: 463.54; Observed: 464.2[M + H]⁺. |
| I-193 | 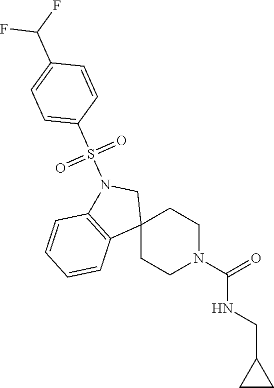 [SMILES: O=C(NCC1CC1)N1CCC2(CC1)CN(S(=O)(=O)c1ccc(C(F)F)cc1)c1ccccc12] | Yield: 37.9 mg, 30.2%; Appearance: Yellow solid; ¹H NMR (600 MHz, DMSO-d₆) δ 7.98 (d, J = 8.1 Hz, 2H), 7.76 (d, J = 8.1 Hz, 2H), 7.49 (d, J = 8.1 Hz, 1H), 7.25-7.21 (m, 1H), 7.19-6.97 (m, 3H), 6.54 (t, J = 5.6 Hz, 1H), 3.90 (s, 2H), 3.83 (d, J = 13.6 Hz, 2H), 2.87 (t, J = 6.1 Hz, 2H), 2.73 (t, J = 13.4, 12.9 Hz, 2H), 1.54-1.45 (m, 2H), 1.05 (d, J = 13.1 Hz, 2H), 0.93-0.84 (m, 1H), 0.39-0.30 (m, 2H), 0.15-0.06 (m, 2H).; HPLC purity: 100%; LCMS Calculated for C₂₄H₂₇F₂N₃O₃S: 475.55; Observed: 476.2[M + H]⁺. |
| I-194 | 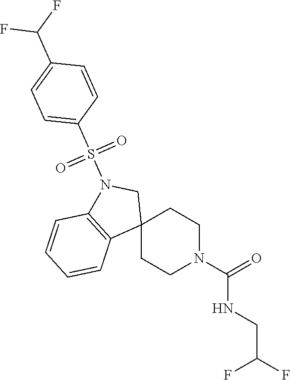 [SMILES: O=C(NCC(F)F)N1CCC2(CC1)CN(S(=O)(=O)c1ccc(C(F)F)cc1)c1ccccc12] | Yield: 33.1 mg, 26.3%; Appearance: Yellow solid; ¹H NMR (400 MHz, DMSO-d₆) δ 7.95 (d, J = 8.1 Hz, 2H), 7.73 (d, J = 8.1 Hz, 2H), 7.53 (d, J = 8.1 Hz, 1H), 7.24-7.15 (m, 1H), 7.09 (d, J = 6.7 Hz, 1H), 7.04-6.78 (m, 3H), 6.03-5.67 (m, 1H), 3.91 (d, J = 13.4 Hz, 2H), 3.87 (s, 2H), 3.42- 3.30 (m, 2H), 2.81 (t, J = 13.0 Hz, 2H), 1.65- 1.54 (m, 2H), 1.18 (d, J = 13.2 Hz, 2H).; HPLC purity: 96.3%; LCMS Calculated for C₂₂H₂₃F₄N₃O₃S: 485.5; Observed: 486.0[M + H]⁺. |
| I-195 | 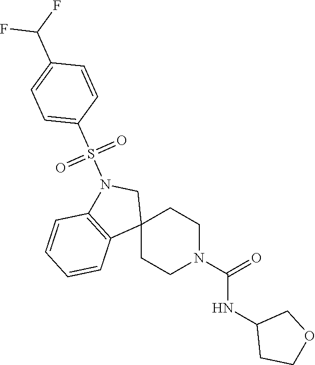 [SMILES: O=C(NC1CCOC1)N1CCC2(CC1)CN(S(=O)(=O)c1ccc(C(F)F)cc1)c1ccccc12] | Yield: 7.8 mg, 6.79%; Appearance: Beige solid; ¹H NMR (600 MHz, DMSO-d₆) δ 7.98 (d, J = 8.2 Hz, 2H), 7.76 (d, J = 8.1 Hz, 2H), 7.49 (d, J = 8.1 Hz, 1H), 7.27-7.20 (m, 1H), 7.20-6.98 (m, 3H), 6.45 (d, J = 6.1 Hz, 1H), 4.16-4.07 (m, 1H), 3.90 (s, 2H), 3.86 (d, J = 12.5 Hz, 2H), 3.78-3.71 (m, 2H), 3.66-3.59 (m, 1H), 3.40 (dd, J = 8.7, 4.7 Hz, 1H), 2.73 (t, J = 13.1 Hz, 2H), 2.05-1.95 (m, 1H), 1.79-1.70 (m, 1H), 1.57-1.41 (m, 2H), 1.06 (d, J = 12.9 Hz, 2H).; HPLC purity: 100%; LCMS Calculated for C₂₄H₂₇F₂N₃O₄S: 491.55; Observed: 492.2[M + H]⁺. |
| I-196 | 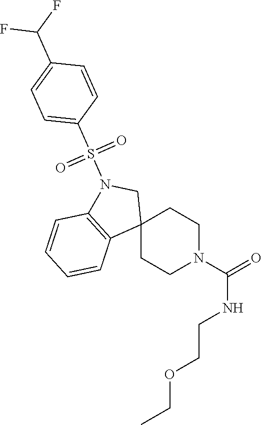 [SMILES: CCOCCNC(=O)N1CCC2(CC1)CN(S(=O)(=O)c1ccc(C(F)F)cc1)c1ccccc12] | Yield: 59.1 mg, 47.1%; Appearance: Yellow solid; ¹H NMR (400 MHz, DMSO-d₆) δ 7.95 (d, J = 8.1 Hz, 2H), 7.73 (d, J = 8.0 Hz, 2H), 7.53 (d, J = 8.0 Hz, 1H), 7.26-7.15 (m, 1H), 7.12-6.80 (m, 3H), 6.34 (t, J = 5.5 Hz, 1H), 3.94-3.88 (m, 1H), 3.88-3.82 (m, 3H), 3.45 (q, J = 7.0 Hz, 2H), 3.40-3.29 (m, 2H), 3.21-3.13 (m, 2H), 2.76 (t, J = 13.0 Hz, 2H), 1.65-1.52 (m, 2H), 1.22-1.12 (m, 5H).; HPLC purity: 100%; LCMS Calculated for C₂₄H₂₉F₂N₃O₄S: 493.57; Observed: 494.2[M + H]⁺. |
| I-197 | 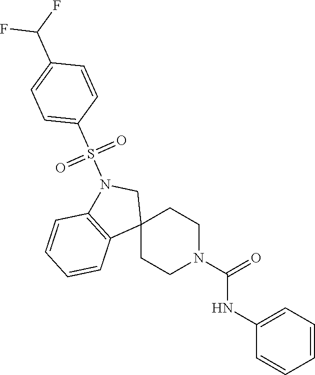 [SMILES: O=C(Nc1ccccc1)N1CCC2(CC1)CN(S(=O)(=O)c1ccc(C(F)F)cc1)c1ccccc12] | Yield: 12.4 mg, 9.83%; Appearance: Brown oil; ¹H NMR (600 MHz, DMSO-d₆) δ 8.48 (s, 1H), 8.00 (d, J = 8.1 Hz, 2H), 7.77 (d, J = 8.1 Hz, 2H), 7.50 (d, J = 8.1 Hz, 1H), 7.47-7.41 (m, 2H), 7.29- 7.08 (m, 5H), 7.07-7.00 (m, 1H), 6.90 (dd, J = 8.0, 6.7 Hz, 1H), 4.02 (d, J = 13.8 Hz, 2H), 3.96 (s, 2H), 2.89 (t, J = 12.9 Hz, 2H), 1.68-1.55 (m, 2H), 1.14 (d, J = 13.0 Hz, 2H).; HPLC purity: 98.48%; LCMS Calculated for C₂₆H₂₅F₂N₃O₃S: 497.56; Observed: 498.2[M + H]⁺. |
| I-198 | 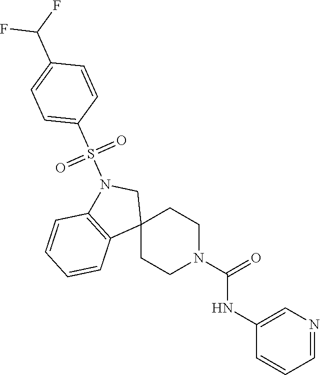 [SMILES: O=C(Nc1cccnc1)N1CCC2(CC1)CN(S(=O)(=O)c1ccc(C(F)F)cc1)c1ccccc12] | Yield: 16.7 mg, 14.4%; Appearance: Yellow solid; ¹H NMR (600 MHz, DMSO-d₆) δ 9.12 (s, 1H), 8.85 (d, J = 2.5 Hz, 1H), 8.31-8.23 (m, 1H), 8.19-8.14 (m, 1H), 8.00 (d, J = 8.2 Hz, 2H), 7.77 (d, J = 8.1 Hz, 2H), 7.56 (dd, J = 8.5, 5.1 Hz, 1H), 7.50 (d, J = 8.1 Hz, 1H), 7.28-6.98 (m, 4H), 4.09- 4.01 (m, 2H), 3.97 (s, 2H), 3.00-2.91 (m, 2H), 1.68-1.57 (m, 2H), 1.17 (d, J = 13.2 Hz, 2H).; HPLC purity: 100%; LCMS Calculated for C₂₅H₂₄F₂N₄O₃S: 498.55; Observed: 499.0[M + H]⁺. |
| I-199 | 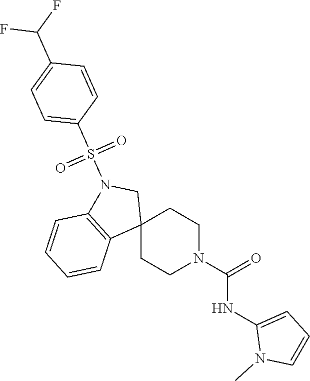 [SMILES: Cn1cccc1NC(=O)N1CCC2(CC1)CN(S(=O)(=O)c1ccc(C(F)F)cc1)c1ccccc12] | Yield: 23.5 mg, 10.9%; Appearance: Pink solid; ¹H NMR (600 MHz, DMSO-d₆) δ 8.08 (s, 1H), 7.99 (d, J = 8.2 Hz, 2H), 7.76 (d, J = 8.2 Hz, 2H), 7.50 (d, J = 8.1 Hz, 1H), 7.24 (td, J = 7.8, 1.3 Hz, 1H), 7.22-6.98 (m, 3H), 6.53 (t, J = 2.4 Hz, 1H), 5.84 (t, J = 3.3 Hz, 1H), 5.66 (dd, J = 3.6, 1.9 Hz, 1H), 3.95 (s, 2H), 3.94-3.89 (m, 2H), 3.33 (s, 3H), 2.93-2.79 (m, 2H), 1.57 (td, J = 13.1, 4.3 Hz, 2H), 1.09 (d, J = 12.9 Hz, 2H); HPLC purity: 97.6%; LCMS Calculated for C₂₅H₂₆F₂N₄O₃S: 500.56; Observed: 501.2[M + H]⁺. |
| I-200 | 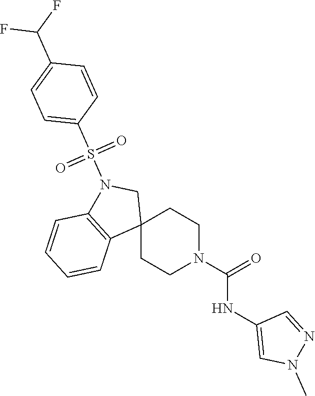 [SMILES: Cn1cc(NC(=O)N2CCC3(CC2)CN(S(=O)(=O)c2ccc(C(F)F)cc2)c2ccccc23)cn1] | Yield: 124.1 mg, 57.6%; Appearance: White solid; ¹H NMR (400 MHz, DMSO-d₆) δ 8.50 (s, 1H), 8.01 (d, J = 8.1 Hz, 2H), 7.78 (d, J = 8.0 Hz, 2H), 7.65 (s, 1H), 7.51 (d, J = 8.1 Hz, 1H), 7.31 (s, 1H), 7.28-7.18 (m, 2H), 7.03 (t, J = 7.5 Hz, 1H), 3.96 (s, 4H), 3.74 (s, 3H), 2.87 (t, J = 13.0 Hz, 2H), 1.57 (t, J = 9.8 Hz, 2H), 1.13 (d, J = 13.1 Hz, 2H); HPLC purity: 100%; LCMS Calculated for C₂₄H₂₅F₂N₅O₃S: 501.55; Observed: 502.0[M + H]⁺. |
| I-201 | 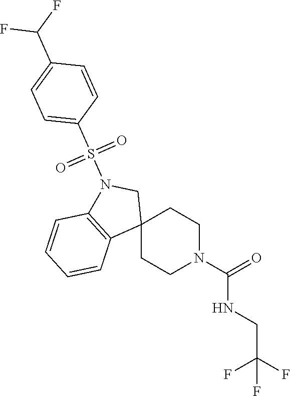 [SMILES: O=C(NCC(F)(F)F)N1CCC2(CC1)CN(S(=O)(=O)c1ccc(C(F)F)cc1)c1ccccc12] | Yield: 99.1 mg, 46.1%; Appearance: White solid; ¹H NMR (400 MHz, DMSO-d₆) δ 8.00 (d, J = 8.0 Hz, 2H), 7.78 (d, J = 8.0 Hz, 2H), 7.51 (d, J = 8.0 Hz, 1H), 7.29-6.94 (m, 5H), 3.94 (s, 2H), 3.92-3.74 (m, 4H), 2.84 (t, J = 13.2 Hz, 2H), 1.68-1.40 (m, 2H), 1.09 (d, J = 13.2 Hz, 2H); HPLC purity: 100%; LCMS Calculated for C₂₂H₂₂F₅N₃O₃S: 503.49; Observed: 504.0[M + H]⁺. |
| I-202 | 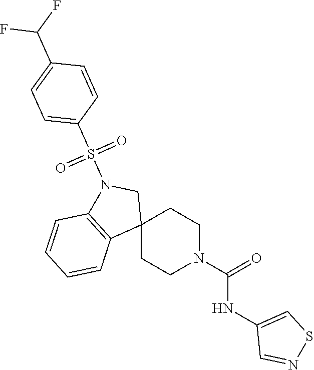 [SMILES: O=C(Nc1cnsc1)N1CCC2(CC1)CN(S(=O)(=O)c1ccc(C(F)F)cc1)c1ccccc12] | Yield: 76.1 mg, 37.3%; Appearance: Beige solid; ¹H NMR (400 MHz, DMSO-d₆) δ 9.22 (s, 1H), 8.59 (d, J = 7.0 Hz, 2H), 8.02 (d, J = 8.1 Hz, 2H), 7.79 (d, J = 8.0 Hz, 2H), 7.52 (d, J = 8.0 Hz, 1H), 7.30-7.20 (m, 2H), 7.04 (t, J = 7.4 Hz, 1H), 4.08-3.90 (m, 4H), 2.94 (t, J = 13.1 Hz, 2H), 1.69-1.53 (m, 2H), 1.17 (d, J = 13.2 Hz, 2H); HPLC purity: 100%; LCMS Calculated for C₂₃H₂₂F₂N₄O₃S₂: 504.57; Observed: 505.2[M + H]⁺. |
| I-203 | 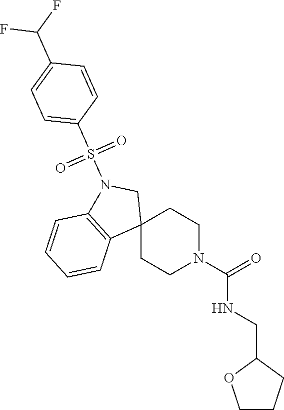 [SMILES: O=C(NCC1CCCO1)N1CCC2(CC1)CN(S(=O)(=O)c1ccc(C(F)F)cc1)c1ccccc12] | Yield: 55.7 mg, 48.5%; Appearance: Yellow solid; ¹H NMR (600 MHz, DMSO-d₆) δ 7.98 (d, J = 8.0 Hz, 2H), 7.76 (d, J = 8.0 Hz, 2H), 7.49 (d, J = 8.1 Hz, 1H), 7.27-7.21 (m, 1H), 7.21-6.97 (m, 3H), 6.51 (t, J = 5.7 Hz, 1H), 3.90 (d, J = 1.6 Hz, 2H), 3.87-3.77 (m, 3H), 3.71 (q, J = 6.8 Hz, 1H), 3.57 (q, J = 7.1 Hz, 1H), 3.03 (q, J = 5.8, 5.4 Hz, 2H), 2.74 (t, J = 13.0 Hz, 2H), 1.87-1.69 (m, 3H), 1.54-1.44 (m, 3H), 1.04 (d, J = 13.1 Hz, 2H).; HPLC purity: 100%; LCMS Calculated for C₂₅H₂₉F₂N₃O₄S: 506.58; Observed: 506.2[M + H]⁺. |
| I-204 | 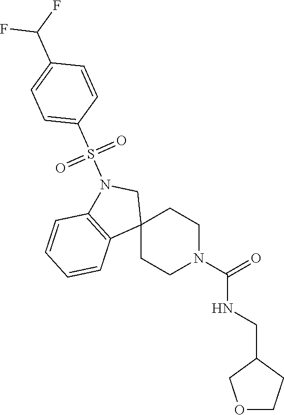 [SMILES: O=C(NCC1CCOC1)N1CCC2(CC1)CN(S(=O)(=O)c1ccc(C(F)F)cc1)c1ccccc12] | Yield: 64.1 mg, 51%; Appearance: Orange solid; ¹H NMR (400 MHz, DMSO-d₆) δ 7.95 (d, J = 8.1 Hz, 2H), 7.73 (d, J = 8.1 Hz, 2H), 7.53 (d, J = 8.1 Hz, 1H), 7.25-7.14 (m, 1H), 7.12-6.79 (m, 3H), 6.54-6.38 (m, 1H), 3.95-3.82 (m, 4H), 3.78-3.67 (m, 1H), 3.67-3.55 (m, 2H), 3.45 (dd, J = 8.6, 5.0 Hz, 1H), 3.12-3.04 (m, 1H), 3.00- 2.87 (m, 1H), 2.75 (t, J = 13.1 Hz, 2H), 2.43- 2.33 (m, 1H), 1.97-1.85 (m, 1H), 1.67-1.50 (m, 3H), 1.17 (d, J = 13.3 Hz, 2H).; HPLC purity: 100%; LCMS Calculated for C₂₅H₂₉F₂N₃O₄S: 506.58; Observed: 506.0[M + H]⁺. |
| I-205 | 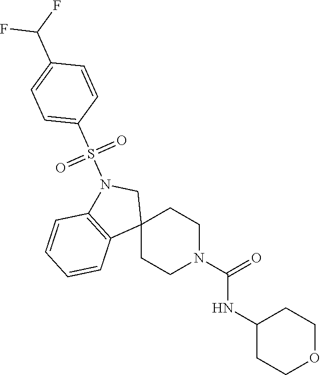 [SMILES: O=C(NC1CCOCC1)N1CCC2(CC1)CN(S(=O)(=O)c1ccc(C(F)F)cc1)c1ccccc12] | Yield: 52.2 mg, 41.5%; Appearance: Light brown solid; ¹H NMR (600 MHz, DMSO-d₆) δ 7.98 (d, J = 8.1 Hz, 2H), 7.76 (d, J = 8.1 Hz, 2H), 7.49 (d, J = 8.1 Hz, 1H), 7.27-7.20 (m, 1H), 7.20- 6.97 (m, 3H), 6.26 (d, J = 7.5 Hz, 1H), 3.90 (s, 2H), 3.83 (dd, J = 27.8, 12.2 Hz, 4H), 3.67-3.55 (m, 1H), 3.29-3.24 (m, 2H), 2.72 (t, J = 13.1 Hz, 2H), 1.68-1.57 (m, 2H), 1.55-1.45 (m, 2H), 1.45-1.36 (m, 2H), 1.06 (d, J = 13.1 Hz, 2H).; HPLC purity: 100%, LCMS Calculated for C₂₅H₂₉F₂N₃O₄S: 506.58; Observed: 506.0[M + H]⁺. |
| I-206 | 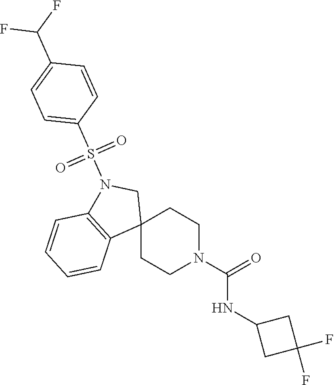 [SMILES: O=C(NC1CC(F)(F)C1)N1CCC2(CC1)CN(S(=O)(=O)c1ccc(C(F)F)cc1)c1ccccc12] | Yield: 46.3 mg, 36.8%; Appearance: Yellow solid; ¹H NMR (600 MHz, DMSO-d₆) δ 7.98 (d, J = 8.1 Hz, 2H), 7.76 (d, J = 8.1 Hz, 2H), 7.49 (d, J = 8.1 Hz, 1H), 7.26-7.20 (m, 1H), 7.20-6.98 (m, 3H), 6.82 (d, J = 6.6 Hz, 1H), 3.99-3.92 (m, 1H), 3.91 (s, 2H), 3.83 (d, J = 13.8 Hz, 2H), 2.83- 2.73 (m, 4H), 2.62-2.52 (m, 2H), 1.56-1.43 (m, 2H), 1.07 (d, J = 13.1 Hz, 2H); HPLC purity: 100%; LCMS Calculated for C₂₄H₂₅F₄N₃O₃S: 511.54; Observed: 512.2[M + H]⁺. |
| I-207 | 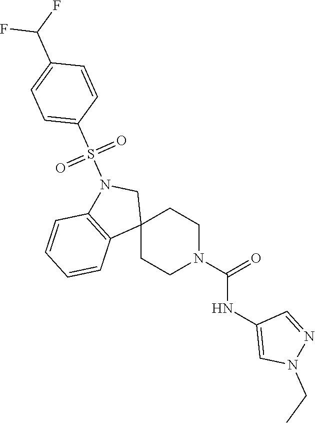 [SMILES: CCn1cc(NC(=O)N2CCC3(CC2)CN(S(=O)(=O)c2ccc(C(F)F)cc2)c2ccccc23)cn1] | Yield: 3.2 mg, 2.55%; Appearance: Yellow solid; ¹H NMR (600 MHz, DMSO-d₆) δ 8.48 (s, 1H), 7.99 (d, J = 8.1 Hz, 2H), 7.77 (d, J = 8.1 Hz, 2H), 7.66 (s, 1H), 7.49 (d, J = 8.1 Hz, 1H), 7.31 (s, 1H), 7.27-7.22 (m, 1H), 7.21-7.00 (m, 3H), 4.01 (q, J = 7.3 Hz, 2H), 3.97-3.85 (m, 4H), 2.85 (t, J = 12.9 Hz, 2H), 1.61-1.50 (m, 2H), 1.29 (t, J = 7.3 Hz, 3H), 1.11 (d, J = 13.2 Hz, 2H).; HPLC purity: 100%; LCMS Calculated for C₂₅H₂₇F₂N₅O₃S: 515.58; Observed: 516.4[M + H]⁺. |
| I-208 | 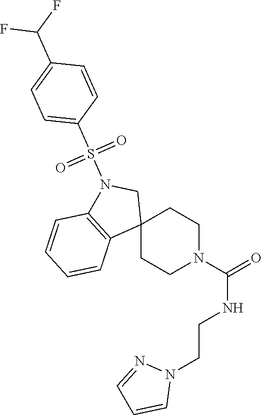 [SMILES: O=C(NCCn1cccn1)N1CCC2(CC1)CN(S(=O)(=O)c1ccc(C(F)F)cc1)c1ccccc12] | Yield: 40.4 mg, 32.1%; Appearance: Yellow solid; ¹H NMR (400 MHz, DMSO-d₆) δ 7.99- 7.90 (m, 2H), 7.73 (d, J = 8.1 Hz, 2H), 7.53 (d, J = 7.4 Hz, 2H), 7.35 (s, 1H), 7.23-7.14 (m, 1H), 7.13-6.78 (m, 3H), 6.58-6.43 (m, 1H), 6.22- 6.09 (m, 1H), 4.22-4.12 (m, 2H), 3.89-3.79 (m, 4H), 3.40 (q, J = 6.0 Hz, 2H), 2.77 (t, J = 13.1 Hz, 2H), 1.64-1.51 (m, 2H), 1.15 (d, J = 13.2 Hz, 2H).; HPLC purity: 100%; LCMS Calculated for C₂₅H₂₇F₂N₅O₃S: 515.58; Observed: 516.0[M + H]⁺. |
| I-209 | 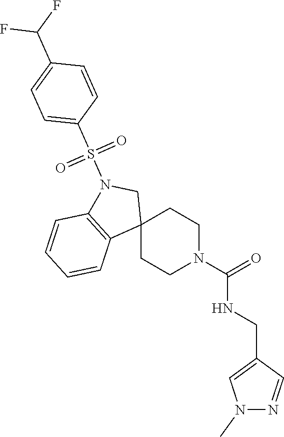 [SMILES: Cn1cc(CNC(=O)N2CCC3(CC2)CN(S(=O)(=O)c2ccc(C(F)F)cc2)c2ccccc23)cn1] | Yield: 47.5 mg, 13.6%; Appearance: Yellow solid; ¹H NMR (600 MHz, DMSO-d₆) δ 7.97 (d, J = 8.1 Hz, 2H), 7.75 (d, J = 8.1 Hz, 2H), 7.51- 7.45 (m, 2H), 7.27 (s, 1H), 7.25-7.21 (m, 1H), 7.19-7.09 (m, 2H), 7.04-6.98 (m, 1H), 4.01 (s, 2H), 3.90 (s, 2H), 3.87-3.80 (m, 2H), 3.75 (s, 3H, 2.77-2.69 (m, 2H), 1.53-1.42 (m, 2H), 1.04 (d, J = 13.5 Hz, 2H).; HPLC purity: 97.92%; LCMS Calculated for C₂₅H₂₇F₂N₅O₃S: 515.58; Observed: 516.0[M + H]⁺. |
| I-210 | 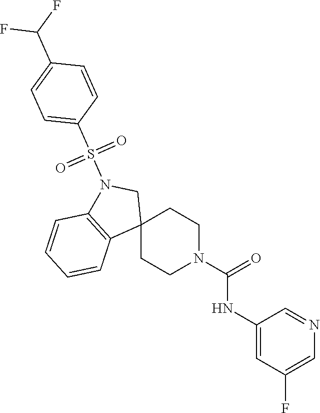 [SMILES: O=C(Nc1cncc(F)c1)N1CCC2(CC1)CN(S(=O)(=O)c1ccc(C(F)F)cc1)c1ccccc12] | Yield: 91.2 mg, 41.4%; Appearance: White solid; ¹H NMR (400 MHz, DMSO-d₆) δ 8.00 (d, J = 8.0 Hz, 2H), 7.78 (d, J = 8.0 Hz, 2H), 7.51 (d, J = 8.0 Hz, 1H), 7.28-6.97 (m, 5H), 3.94 (d, J = 1.9 Hz, 2H), 3.92-3.75 (m, 4H), 2.84 (t, J = 13.2 Hz, 2H), 1.61-1.45 (m, 2H), 1.09 (d, J = 13.2 Hz, 2H); HPLC purity: 100%; LCMS Calculated for C₂₅H₂₃F₃N₄O₃S: 516.54; Observed: 517.2[M + H]⁺. |
| I-211 | 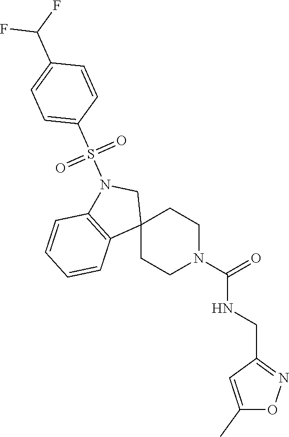 [SMILES: Cc1cc(CNC(=O)N2CCC3(CC2)CN(S(=O)(=O)c2ccc(C(F)F)cc2)c2ccccc23)no1] | Yield: 16.9 mg, 4.87%; Appearance: Yellow solid; ¹H NMR (600 MHz, DMSO-d₆) δ 7.98 (d, J = 8.2 Hz, 2H), 7.75 (d, J = 8.1 Hz, 2H), 7.49 (d, J = 8.1 Hz, 1H), 7.25-7.19 (m, 1H), 7.20-6.97 (m, 4H), 6.06 (s, 1H), 4.17 (d, J = 5.7 Hz, 2H), 3.91 (s, 2H), 3.83 (d, J = 13.8 Hz, 2H), 2.83-2.73 (m, 2H), 2.34 (s, 3H), 1.57-1.44 (m, 2H), 1.05 (d, J = 13.3 Hz, 2H).; HPLC purity: 100%; LCMS Calculated for C₂₅H₂₆F₂N₄O₄S: 516.56; Observed: 517.0[M + H]⁺. |
| I-212 | 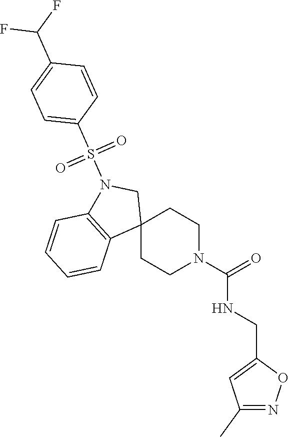 [SMILES: Cc1cc(CNC(=O)N2CCC3(CC2)CN(S(=O)(=O)c2ccc(C(F)F)cc2)c2ccccc23)on1] | Yield: 28.5 mg, 20.6%; Appearance: Yellow solid; ¹H NMR (600 MHz, DMSO-d₆) δ 7.98 (d, J = 8.1 Hz, 2H), 7.76 (d, J = 8.1 Hz, 2H), 7.49 (d, J = 8.1 Hz, 1H), 7.26-7.20 (m, 1H), 7.19-6.99 (m, 4H), 6.08 (s, 1H), 4.26 (s, 2H), 3.92 (s, 2H), 3.84 (d, J = 13.8 Hz, 2H), 2.83-2.74 (m, 2H), 2.17 (s, 3H), 1.56-1.48 (m, 2H), 1.06 (d, J = 13.1 Hz, 2H).; HPLC purity: 100%; LCMS Calculated for C₂₅H₂₆F₂N₄O₄S: 516.56; Observed: 517.2[M + H]⁺. |
| I-213 | 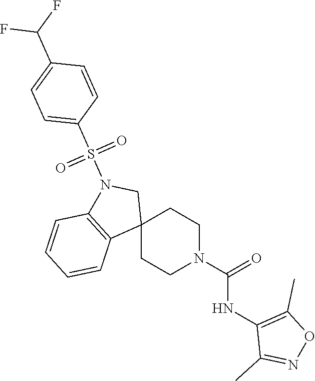 [SMILES: Cc1noc(C)c1NC(=O)N1CCC2(CC1)CN(S(=O)(=O)c1ccc(C(F)F)cc1)c1ccccc12] | Yield: 5.8 mg, 4.51%; Appearance: Yellow solid; ¹H NMR (600 MHz, DMSO-d₆) δ 7.99 (d, J = 8.2 Hz, 2H), 7.90 (s, 1H), 7.76 (d, J = 8.1 Hz, 2H), 7.50 (d, J = 8.1 Hz, 1H), 7.28-7.22 (m, 1H), 7.22-7.00 (m, 3H), 3.95 (s, 2H), 3.91 (d, J = 14.0 Hz, 2H), 2.90 (t, J = 13.1 Hz, 2H), 2.20 (s, 3H), 2.04 (s, 3H), 1.65-1.54 (m, 2H), 1.12 (d, J = 12.9 Hz, 2H).; HPLC purity: 95.14%; LCMS Calculated for C₂₅H₂₆F₂N₄O₄S: 516.56; Observed: 517.0[M + H]⁺. |
| I-214 | 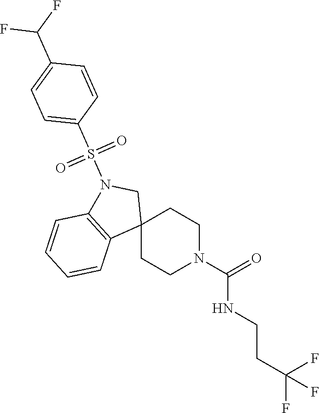 [SMILES: O=C(NCCC(F)(F)F)N1CCC2(CC1)CN(S(=O)(=O)c1ccc(C(F)F)cc1)c1ccccc12] | Yield: 50.5 mg, 39.2%; Appearance: Yellow solid; ¹H NMR (400 MHz, DMSO-d₆) δ 7.95 (d, J = 8.1 Hz, 2H), 7.73 (d, J = 8.1 Hz, 2H), 7.53 (d, J = 8.1 Hz, 1H), 7.25-7.16 (m, 1H), 7.12-6.78 (m, 3H), 6.60 (t, J = 5.5 Hz, 1H), 3,93-3.80 (m, 2H), 3.25 (q, J = 6.7 Hz, 2H), 2.78 (t, J = 12.9 Hz, 2H), 2.43-2.29 (m, 2H), 1.66-1.51 (m, 2H), 1.18 (d, J = 13.3 Hz, 2H).; HPLC purity: 100%; LCMS Calculated for C₂₃H₂₄F₅N₃O₃S: 517.52; Observed: 518.0[M + H]⁺. |
| I-215 | 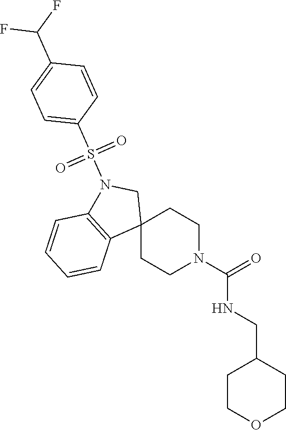 [SMILES: O=C(NCC1CCOCC1)N1CCC2(CC1)CN(S(=O)(=O)c1ccc(C(F)F)cc1)c1ccccc12] | Yield: 53.5 mg, 42.3%; Appearance: Light brown solid; ¹H NMR (400 MHz, DMSO-d₆) δ 7.95 (d, J = 8.1 Hz, 2H), 7.73 (d, J = 8.1 Hz, 2H), 7.53 (d, J = 8.1 Hz, 1H), 7.23-7.14 (m, 1H), 7.13- 6.81 (m, 3H), 6.36 (t, J = 5.7 Hz, 1H), 3.93- 3.75 (m, 7H), 3.26 (t, J = 11.7 Hz, 2H), 3.19 (d, J = 5.3 Hz, 1H), 2.91 (t, J = 6.2 Hz, 2H), 2.74 (t, J = 12.9 Hz, 2H), 1.72-1.61 (m, 1H), 1.57 (d, J = 12.8 Hz, 4H), 1.22-1.07 (m, 4H).; HPLC purity: 100%; LCMS Calculated for C₂₆H₃₁F₂N₃O₄S: 519.61; Observed: 520.0[M + H]⁺. |
| I-216 | 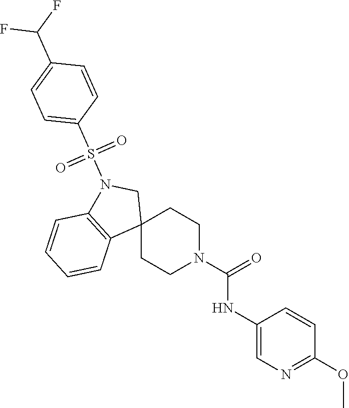 [SMILES: COc1ccc(NC(=O)N2CCC3(CC2)CN(S(=O)(=O)c2ccc(C(F)F)cc2)c2ccccc23)cn1] | Yield: 33.5 mg, 28.6%; Appearance: Yellow solid; ¹H NMR (600 MHz, DMSO-d₆) δ 8.48 (s, 1H), 8.15 (d, J = 2.7 Hz, 1H), 8.00 (d, J = 8.1 Hz, 2H), 7.77 (d, J = 8.1 Hz, 2H), 7.73 (dd, J = 8.9, 2.7 Hz, 1H), 7.50 (d, J = 8.1 Hz, 1H), 7.27-7.00 (m, 4H), 6.71 (d, J = 8.8 Hz, 1H), 4.00 (d, J = 13.8 Hz, 2H), 3.96 (s, 2H), 2.90 (t, J = 13.3 Hz, 2H), 1.65-1.54 (m, 2H), 1.14 (d, J = 13.1 Hz, 2H).; HPLC purity: 100%; LCMS Calculated for C₂₆H₂₆F₂N₄O₄S: 528.57; Observed: 529.0[M + H]⁺. |
| I-217 | 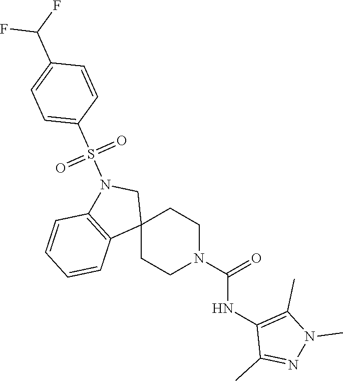 [SMILES: Cc1nn(C)c(C)c1NC(=O)N1CCC2(CC1)CN(S(=O)(=O)c1ccc(C(F)F)cc1)c1ccccc12] | Yield: 90 mg, 39.7%; Appearance: White solid; ¹H NMR (400 MHz, DMSO-d₆) δ 8.01 (d, J = 8.1 Hz, 2H), 7.78 (d, J = 8.1 Hz, 2H), 7.62 (s, 1H), 7.52 (d, J = 8.0 Hz, 1H), 7.31-7.23 (m, 1H), 7.19 (d, J = 7.6, 1.4 Hz, 1H), 7.14-6.95 (m, 2H), 3,92 (s, 4H), 3.60 (s, 3H), 2.93-2.80 (m, 2H), 2.01 (s, 3H), 1.93 (s, 3H), 1.64-1.52 (m, 2H), 1.11 (d, J = 13.1 Hz, 2H); HPLC purity: 100%; LCMS Calculated for C₂₆H₂₉F₂N₅O₃S: 529.61; Observed: 530.0[M + H]⁺. |
| I-218 | 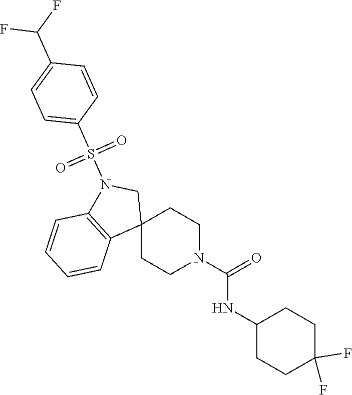 [SMILES: O=C(NC1CCC(F)(F)CC1)N1CCC2(CC1)CN(S(=O)(=O)c1ccc(C(F)F)cc1)c1ccccc12] | Yield: 48 mg, 41%; Appearance: Yellow solid; ¹H NMR (600 MHz, DMSO-d₆) δ 7.98 (d, J = 8.5 Hz, 2H), 7.76 (d, J = 8.1 Hz, 2H), 7.49 (d, J = 8.2 Hz, 1H), 7.26-6.96 (m, 4H), 6.25 (d, J = 7.7 Hz, 1H), 3.90 (s, 2H), 3.84 (d, J = 13.7 Hz, 2H), 3.66-3.51 (m, 1H), 2.72 (t, J = 13.4 Hz, 2H), 2.02-1.91 (m, 2H), 1.91-1.72 (m, 4H), 1.55- 1.41 (m, 4H), 1.06 (d, J = 13.1 Hz, 2H); HPLC purity: 100%; LCMS Calculated for C₂₆H₂₉F₄N₃O₃S: 539.59; Observed: 540.0[M + H]⁺. |
| I-219 | 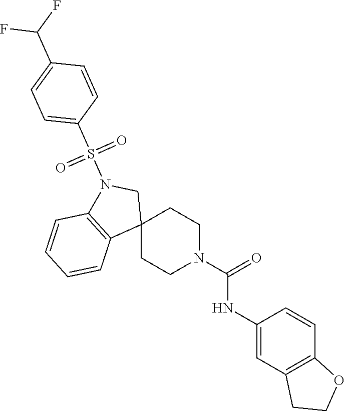 [SMILES: O=C(Nc1ccc2c(c1)CCO2)N1CCC2(CC1)CN(S(=O)(=O)c1ccc(C(F)F)cc1)c1ccccc12] | Yield: 38.3 mg, 14.2%; Appearance: Light brown solid; ¹H NMR (400 MHz, DMSO-d₆) δ 8.14 (s, 1H), 7.97 (d, J = 8.1 Hz, 2H), 7.74 (d, J = 7.9 Hz, 2H), 7.54 (d, J = 8.1 Hz, 1H), 7.30 (s, 1H), 7.27-7.16 (m, 1H), 7.17-6.81 (m, 4H), 6.55 (d, J = 8.5 Hz, 1H), 4.48 (dd, J = 9.9, 7.3 Hz, 2H), 4.05 (d, J = 13.8 Hz, 2H), 3.90 (s, 2H), 3.22-3.11 (m, 2H), 2.87 (t, J = 13.1 Hz, 2H), 1.73-1.60 (m, 2H), 1.23 (d, J = 13.0 Hz, 2H).; HPLC purity: 97.98%; LCMS Calculated for C₂₈H₂₇F₂N₃O₄S: 539.6; Observed: 540.2[M + H]⁺. |
| I-220 | 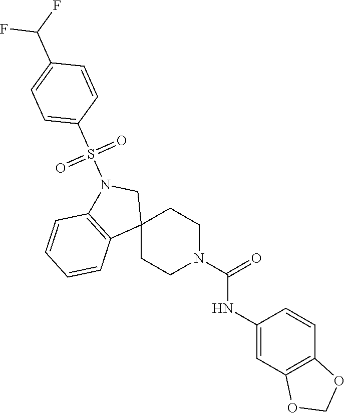 [SMILES: O=C(Nc1ccc2c(c1)OCO2)N1CCC2(CC1)CN(S(=O)(=O)c1ccc(C(F)F)cc1)c1ccccc12] | Yield: 29.3 mg, 27.2%; Appearance: Yellow solid; ¹H NMR (600 MHz, DMSO-d₆) δ 8.37 (s, 1H), 7.99 (d, J = 8.2 Hz, 2H), 7.77 (d, J = 8.1 Hz, 2H), 7.50 (d, J = 8.1 Hz, 1H), 7.27-7.00 (m, 5H), 6.80 (dd, J = 8.4, 2.1 Hz, 1H), 6.75 (d, J = 8.4 Hz, 1H), 5.92 (s, 2H), 3.98 (d, J = 13.8 Hz, 2H), 3.95 (s, 2H), 2.87 (t, J = 12.8 Hz, 2H), 1.63-1.54 (m, 2H), 1.12 (d, J = 13.0 Hz, 2H).; HPLC purity: 98.07%; LCMS Calculated for C₂₇H₂₅F₂N₃O₅S: 541.57; Observed: 542.0[M + H]⁺. |
| I-221 | 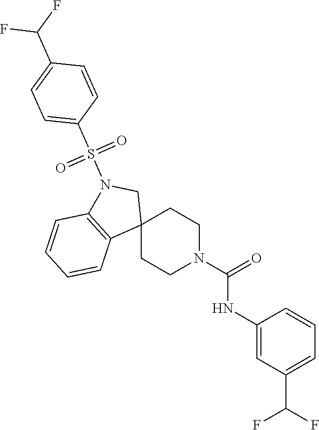 [SMILES: O=C(Nc1cccc(C(F)F)c1)N1CCC2(CC1)CN(S(=O)(=O)c1ccc(C(F)F)cc1)c1ccccc12] | Yield: 8.8 mg, 8.78%; Appearance: Yellow solid; ¹H NMR (600 MHz, DMSO-d₆) δ 8.71 (s, 1H), 8.00 (d, J = 8.1 Hz, 2H), 7.77 (d, J = 8.1 Hz, 2H), 7.73 (s, 1H), 7.59 (d, J = 8.3 Hz, 1H), 7.50 (d, J = 8.2 Hz, 1H), 7.37-7.32 (m, 1H), 7.26- 7.08 (m, 4H), 7.04-6.85 (m, 2H), 4.03 (d, J = 13.8 Hz, 2H), 3.96 (s, 2H), 2.91 (t, J = 13.1 Hz, 2H), 1.66-1.55 (m, 2H), 1.15 (d, J = 13.1 Hz, 2H).; HPLC purity: 96.42%; LCMS Calculated for C₂₇H₂₅F₄N₃O₃S: 547.57; Observed: 548.2[M + H]⁺. |
| I-222 | 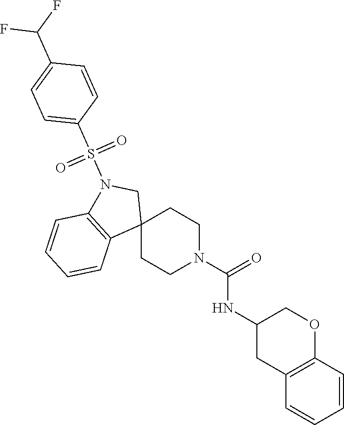 [SMILES: O=C(NC1COc2ccccc2C1)N1CCC2(CC1)CN(S(=O)(=O)c1ccc(C(F)F)cc1)c1ccccc12] | Yield: 32.2 mg, 28.7%; Appearance: Light brown solid; ¹H NMR (400 MHz, DMSO-d₆) δ 7.96 (d, J = 8.0 Hz, 2H), 7.73 (d, J = 8.1 Hz, 2H), 7.53 (d, J = 8.1 Hz, 1H), 7.25-7.14 (m, 1H), 7.14- 6.93 (m, 5H), 6.89-6.74 (m, 1H), 6.74-6.67 (m, 1H), 6.27 (d, J = 7.1 Hz, 1H), 4.22-4.10 (m, 1H), 4.10-3.99 (m, 1H), 3.93 (d, J = 13.8 Hz, 2H), 3.87 (s, 2H), 3.82-3.66 (m, 2H), 3.20 (d, J = 5.2 Hz, 2H), 2.91 (dd, J = 16.3, 5.5 Hz, 1H), 2.85-2.70 (m, 3H), 1.70-1.51 (m, 2H), 1.19 (d, J = 13.3 Hz, 2H).; HPLC purity: 100%; LCMS Calculated for C₂₉H₂₉F₂N₃O₄S: 53.62; Observed: 554.2[M + H]⁺. |
| I-223 | 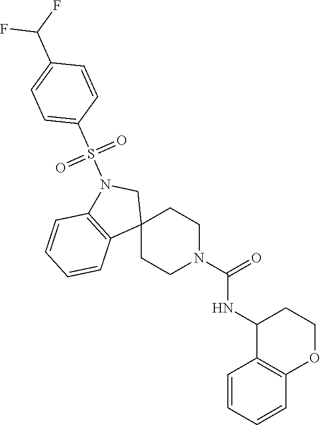 [SMILES: O=C(NC1CCOc2ccccc21)N1CCC2(CC1)CN(S(=O)(=O)c1ccc(C(F)F)cc1)c1ccccc12] | Yield: 37.3 mg, 31%; Appearance: Light brown solid; ¹H NMR (400 MHz, DMSO-d₆) δ 7.95 (d, J = 8.1 Hz, 2H), 7.73 (d, J = 8.1 Hz, 2H), 7.53 (d, J = 8.1 Hz, 1H), 7.25-7.16 (m, 2H), 7.14-6.79 (m, 5H), 6.76 (d, J = 8.2 Hz, 1H), 6.70 (d, J = 8.3 Hz, 1H), 4.93 (q, J = 6.7 Hz, 1H), 4.32-4.13 (m, 2H), 3.97 (d, J = 13.7 Hz, 2H), 2.79 (t, J = 13.0 Hz, 2H), 2.12-1.91 (m, 2H), 1.69-1.54 (m, 2H), 1.19 (d, J = 13.3 Hz, 2H).; HPLC purity: 100%; LCMS Calculated for C₂₉H₂₉F₂N₃O₄S: 554.62; Observed: 554.0[M + H]⁺. |
| I-224 | 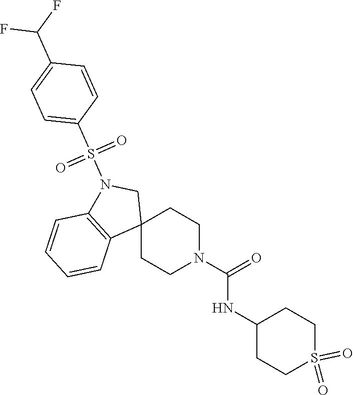 [SMILES: O=C(NC1CCS(=O)(=O)CC1)N1CCC2(CC1)CN(S(=O)(=O)c1ccc(C(F)F)cc1)c1ccccc12] | Yield: 32 mg, 27.8%; Appearance: Light brown solid; ¹H NMR (400 MHz, DMSO-d₆) δ 7.95 (d, J = 8.1 Hz, 2H), 7.73 (d, J = 8.0 Hz, 2H), 7.53 (d, J = 8.0 Hz, 1H), 7.25-7.16 (m, 1H), 7.14-6.79 (m, 3H), 6.33 (d, J = 7.8 Hz, 1H), 3.92 (d, J = 14.4 Hz, 2H), 3.86 (s, 2H), 3.22-3.08 (m, 2H), 3.02- 2.97 (m, 2H), 2.75 (t, J = 13.0 Hz, 2H), 2.13- 1.95 (m, 4H), 1.63-1.50 (m, 2H), 1.18 (d, J = 13.2 Hz, 2H).; HPLC purity: 100%; LCMS Calculated for C₂₅H₂₉F₂N₃O₅S₂: 553.64; Observed: 554.0[M + H]⁺. |
| I-225 | 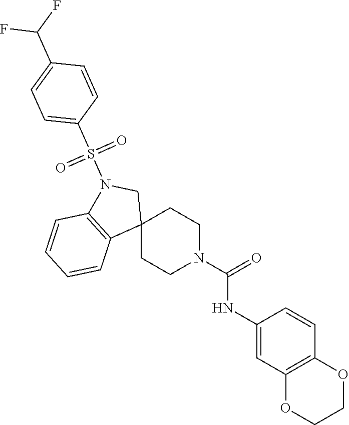 [SMILES: O=C(Nc1ccc2c(c1)OCCO2)N1CCC2(CC1)CN(S(=O)(=O)c1ccc(C(F)F)cc1)c1ccccc12] | Yield: 0.0441 mg, 37.3%; Appearance: Light brown solid; ¹H NMR (400 MHz, DMSO-d₆) δ 8.17 (s, 1H), 7.97 (d, J = 8.1 Hz, 2H), 7.74 (d, J = 8.1 Hz, 2H), 7.54 (d, J = 8.1 Hz, 1H), 7.24-7.17 (m, 1H), 7.16-6.79 (m, 5H), 6.62 (d, J = 8.7 Hz, 1H), 4.26-4.14 (m, 4H), 4.05 (d, J = 13.9 Hz, 2H), 3.90 (s, 2H), 2.87 (t, J = 13.0 Hz, 2H), 1.66 (t, J = 12.1 Hz, 2H), 1.23 (d, J = 13.3 Hz, 2H); HPLC purity: 96%; LCMS Calculated for C₂₈H₂₇F₂N₃O₅S: 555.6; Observed: 556.2[M + H]⁺. |
| I-226 | 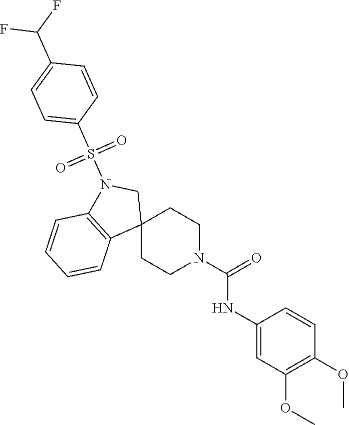 [SMILES: COc1ccc(NC(=O)N2CCC3(CC2)CN(S(=O)(=O)c2ccc(C(F)F)cc2)c2ccccc23)cc1OC] | Yield: 44.5 mg, 39%; Appearance: Light brown solid; ¹H NMR (600 MHz, DMSO-d₆) δ 8.31 (d, J = 15.6 Hz, 1H), 8.00 (d, J = 8.1 Hz, 2H), 7.77 (d, J = 8.1 Hz, 2H), 7.50 (d, J = 8.1 Hz, 1H), 7.29- 7.08 (m, 4H), 7.05-7.00 (m, 1H), 6.95 (dd, J = 8.7, 2.4 Hz, 1H), 6.79 (d, J = 8.7 Hz, 1H), 4.00 (d, J = 13.7 Hz, 2H), 3.95 (s, 2H), 3.68 (d, J = 6.5 Hz, 6H), 2.87 (t, J = 12.7 Hz, 2H), 1.65-1.53 (m, 2H), 1.13 (d, J = 13.1 Hz, 2H).; HPLC purity: 100%; LCMS Calculated for C₂₈H₂₉F₂N₃O₅S: 557.61; Observed: 558.0[M + H]⁺. |
| I-227 | 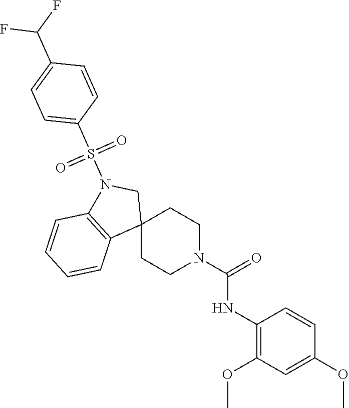 [SMILES: COc1ccc(NC(=O)N2CCC3(CC2)CN(S(=O)(=O)c2ccc(C(F)F)cc2)c2ccccc23)c(OC)c1] | Yield: 5.4 mg, 4.66%; Appearance: Yellow solid; ¹H NMR (600 MHz, DMSO-d₆) δ 7.99 (d, J = 8.2 Hz, 2H), 7.77 (d, J = 8.1 Hz, 2H), 7.54 (s, 1H), 7.50 (d, J = 8.1 Hz, 1H), 7.29 (d, J = 8.6 Hz, 1H), 7.27-7.22 (m, 1H), 7.22-6.99 (m, 3H), 6.55 (d, J = 2.6 Hz, 1H), 6.43 (dd, J = 8.7, 2.7 Hz, 1H), 3.94 (s, 2H), 3.92 (d, J = 13.5 Hz, 2H), 3.75 (s, 3H), 3.72 (s, 3H), 2.87 (t, J = 13.0 Hz, 2H), 1.64-1.51 (m, 2H), 1.10 (d, J = 13.2 Hz, 2H); HPLC purity: 100%; LCMS Calculated for C₂₈H₂₉F₂N₃O₅S: 557.61; Observed: 558.2[M + H]⁺. |
| I-228 | 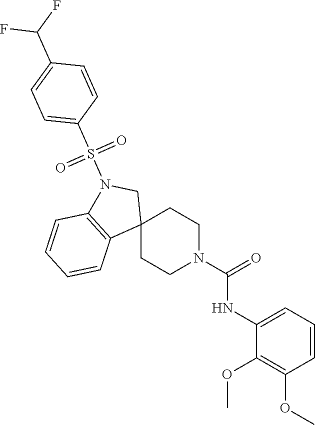 [SMILES: COc1cccc(NC(=O)N2CCC3(CC2)CN(S(=O)(=O)c2ccc(C(F)F)cc2)c2ccccc23)c1OC] | Yield: 45.5 mg, 40.3%; Appearance: Beige solid: ¹H NMR (400 MHz, DMSO-d₆) δ 7.97 (d, J = 8.1 Hz, 2H), 7.74 (d, J = 8.1 Hz, 2H), 7.59 (s, 1H), 7.54 (d, J = 8.1 Hz, 1H), 7.40 (d, J = 8.2 Hz, 1H), 7.26-7.19 (m, 1H), 7.15-6.82 (m, 4H), 6.61 (d, J = 8.5 Hz, 1H), 3.97 (d, J = 13.6 Hz, 2H), 3.91 (s, 2H), 3.83 (s, 3H), 3.78 (s, 3H), 2.96 (d, J = 13.0 Hz, 2H), 1.79-1.62 (m, 2H), 1.24 (d, J = 13.2 Hz, 2H).; HPLC purity: 100%; LCMS Calculated for C₂₈H₂₉F₂N₃O₅S: 557.61; Observed: 558.2[M + H]⁺. |
| I-229 | 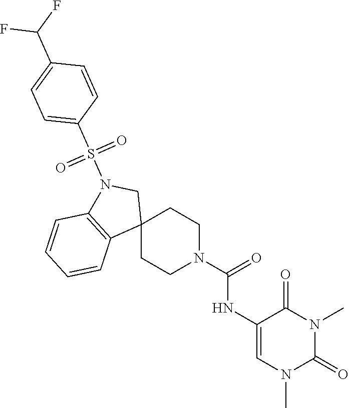 [SMILES: Cn1cc(NC(=O)N2CCC3(CC2)CN(S(=O)(=O)c2ccc(C(F)F)cc2)c2ccccc23)c(=O)n(C)c1=O] | Yield: 1.9 mg, 1.85%; Appearance: White solid; ¹H NMR (600 MHz, DMSO-d₆) δ 7.99 (d, J = 8.2 Hz, 2H), 7.87 (s, 1H), 7.76 (d, J = 8.2 Hz, 2H), 7.58 (s, 1H), 7.49 (d, J = 8.1 Hz, 1H), 7.26-7.21 (m, 1H), 7.20-6.99 (m, 3H), 3.94 (s, 2H), 3.92- 3.83 (m, 2H), 3.30 (s, 3H), 3.19 (s, 3H), 2.89 (t, J = 12.9 Hz, 2H), 1.63-1.51 (m, 2H), 1.12 (d, J = 13.0 Hz, 2H).; HPLC purity: 100%; LCMS Calculated for C₂₆H₂₇F₂N₅O₅S: 559.59; Observed: 560.2[M + H]⁺. |
| I-230 | 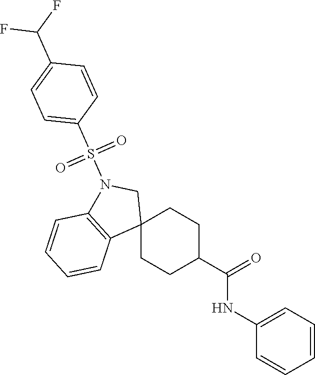 [SMILES: O=C(Nc1ccccc1)C1CCC2(CC1)CN(S(=O)(=O)c1ccc(C(F)F)cc1)c1ccccc12] | Yield: 14.1 mg, 11.5%; Appearance: Brown oil; ¹H NMR (600 MHz, DMSO-d₆) δ 9.81 (d, J = 6.9 Hz, 1H), 8.03-7.95 (m, 2H), 7.80-7.72 (m, 2H), 7.63-7.57 (m, 2H), 7.50 (d, J = 8.1 Hz, 1H), 7.30-7.23 (m, 3H), 7.18 (d, J = 7.0 Hz, 1H), 7.11- 6.97 (m, 3H), 3.88-3.73 (m, 2H), 2.44-2.27 (m, 1H), 1.89-1.62 (m, 3H), 1.58-1.43 (m, 3H), 1.28-1.10 (m, 2H).; HPLC purity: 100%; LCMS Calculated for C₂₇H₂₆F₂N₂O₃S: 496.57; Observed: 497.0[M + H]⁺. |
| I-231 | 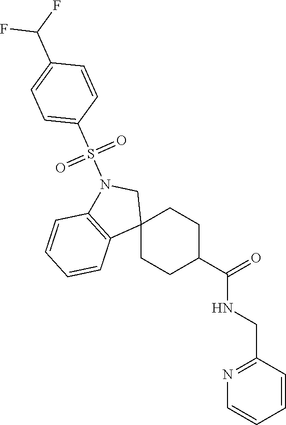 [SMILES: O=C(NCc1ccccn1)C1CCC2(CC1)CN(S(=O)(=O)c1ccc(C(F)F)cc1)c1ccccc12] | Yield: 19.1 mg, 15.2%; Appearance: Yellow solid; ¹H NMR (600 MHz, DMSO-d₆) δ 8.47 (dd, J = 11.1, 4.6 Hz, 1H), 8.42-8.31 (m, 1H), 7.97 (dd, J = 8.3, 4.1 Hz, 2H), 7.80-7.69 (m, 3H), 7.49 (dd, J = 8.0, 2.3 Hz, 1H), 7.28-7.19 (m, 3H), 7.19-6.97 (m, 3H), 4.34 (d, J = 6.0 Hz, 2H), 3.85- 3.67 (m, 2H), 2.43-2.21 (m, 0H), 2.30-2.15 (m, 1H), 1.86-1.76 (m, 1H), 1.68-1.63 (m, 1H), 1.63-1.43 (m, 4H), 1.21-1.08 (m, 2H).; HPLC purity: 100%; LCMS Calculated for C₂₇H₂₇F₂N₃O₃S: 511.59; Observed: 512.2[M + H]⁺. |
| I-232 | 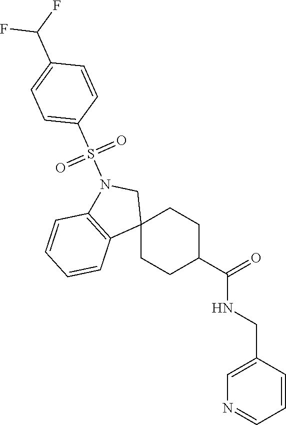 [SMILES: O=C(NCc1cccnc1)C1CCC2(CC1)CN(S(=O)(=O)c1ccc(C(F)F)cc1)c1ccccc12] | Yield: 16.9 mg, 17.7%; Appearance: Beige solid; ¹H NMR (600 MHz, DMSO-d₆) δ 8.47- 8.40 (m, 2H), 8.38-8.32 (m, 1H), 7.97 (dd, J = 8.3, 5.6 Hz, 2H), 7.75 (dd, J = 8.5, 3.3 Hz, 2H), 7.63-7.59 (m, 1H), 7.49 (dd, J = 8.1, 3.0 Hz, 1H), 7.36-7.30 (m, 1H), 7.24-7.19 (m, 1H), 7.18- 6.97 (m, 3H), 4.33-4.24 (m, 2H), 3.84-3.71 (m, 2H), 2.24-2.13 (m, 1H), 1.84-1.43 (m, 6H), 1.19-1.08 (m, 2H).; HPLC purity: 100%; LCMS Calculated for C₂₇H₂₇F₂N₃O₃S: 511.59; Observed: 512.2[M + H]⁺. |
| I-233 | 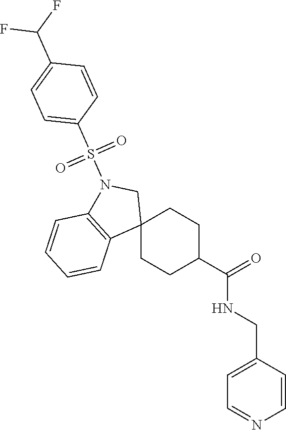 [SMILES: O=C(NCc1ccncc1)C1CCC2(CC1)CN(S(=O)(=O)c1ccc(C(F)F)cc1)c1ccccc12] | Yield: 15 mg, 19.5%; Appearance: Yellow solid; ¹H NMR (600 MHz, DMSO-d₆) δ 8.51- 8.44 (m, 2H), 8.42-8.36 (m, 1H), 7.97 (dd, J = 8.1, 5.4 Hz, 2H), 7.75 (dd, J = 8.3, 4.1 Hz, 2H), 7.49 (dd, J = 8.1, 3.0 Hz, 1H), 7.25-7.19 (m, 3H), 7.18-6.96 (m, 3H), 4.27 (t, J = 5.8 Hz, 2H), 3.86- 3.71 (m, 2H), 2.41-2.19 (m, 1H), 1.86-1.76 (m, 1H), 1.68-1.63 (m, 1H), 1.61-1.43 (m, 4H), 1.21-1.08 (m, 2H).; HPLC purity: 97.6%; LCMS Calculated for C₂₇H₂₇F₂N₃O₃S: 511.59; Observed: 512.2[M + H]⁺. |
| I-234 | 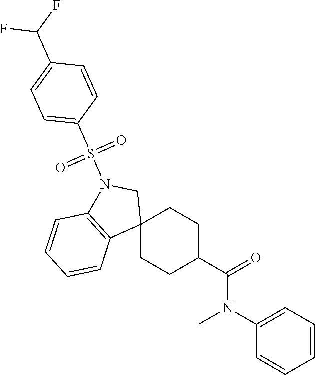 [SMILES: CN(C(=O)C1CCC2(CC1)CN(S(=O)(=O)c1ccc(C(F)F)cc1)c1ccccc12)c1ccccc1] | Yield: 5.8 mg, 5.51%; Appearance: Yellow solid; ¹H NMR (600 MHz, DMSO-d₆) δ 8.00- 7.88 (m, 2H), 7.73 (d, J = 7.7 Hz, 2H), 7.50-7.41 (m, 3H), 7.37 (s, 1H), 7.30 (d, J = 7.6 Hz, 2H), 7.26-6.89 (m, 4H), 3.83-3.55 (m, 2H), 3.12 (s, 3H), 2.35-2.10 (m, 1H), 1.73-1.32 (m, 5H), 1.23-1.08 (m, 2H), 1.07-0.96 (m, 2H).; HPLC purity: 100%; LCMS Calculated for C₂₈H₂₈F₂N₂O₃S: 510.6; Observed: 511.2[M + H]⁺. |
| I-235 | 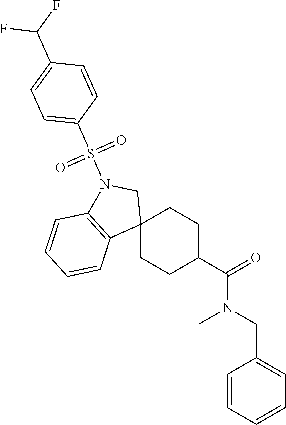 [SMILES: CN(Cc1ccccc1)C(=O)C1CCC2(CC1)CN(S(=O)(=O)c1ccc(C(F)F)cc1)c1ccccc12] | Yield: 43 mg, 39.6%; Appearance: Yellow solid; ¹H NMR (600 MHz, DMSO-d₆) δ 8.02- 7.92 (m, 2H), 7.78-7.72 (m, 2H), 7.52-7.45 (m, 1H), 7.38-7.34 (m, 1H), 7.34-7.26 (m, 2H), 7.26-7.13 (m, 4H), 7.12-6.97 (m, 2H), 4.65- 4.47 (m, 2H), 3.86-3.64 (m, 2H), 3.15 (dd, J = 5.3, 2.3 Hz, 1H), 2.93 (d, J = 8.7 Hz, 2H), 2.86- 2.64 (m, 2H), 1.79-1.66 (m, 1H), 1.63-1.46 (m, 4H), 1.34-1.01 (m, 2H).; HPLC purity: 100%; LCMS Calculated for C₂₉H₃₀F₂N₂O₃S: 524.63; Observed: 525.2[M + H]⁺. |
| I-236 | 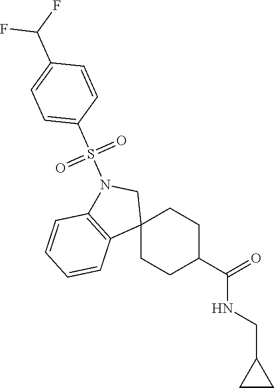 [SMILES: O=C(NCC1CC1)C1CCC2(CC1)CN(S(=O)(=O)c1ccc(C(F)F)cc1)c1ccccc12] | Yield: 33.9 mg, 34.6%; Appearance: White solid; ¹H NMR (400 MHz, DMSO-d₆) δ 7.96- 7.87 (m, 2H), 7.71 (d, J = 8.1 Hz, 2H), 7.60-7.47 (m, 2H), 7.26-6.87 (m, 4H), 3.84-3.64 (m, 2H), 2.95 (t, J = 6.1 Hz, 2H), 2.34-2.01 (m, 1H), 1.88- 1.40 (m, 6H), 1.27-1.16 (m, 2H), 0.97-0.83 (m, 1H), 0.45-0.32 (m, 2H), 0.17 (d, J = 5.2 Hz, 2H).; HPLC purity: 100%; LCMS Calculated for C₂₅H₂₈F₂N₂O₃S: 474.57; Observed: 475.2[M + H]⁺. |
| I-237 | 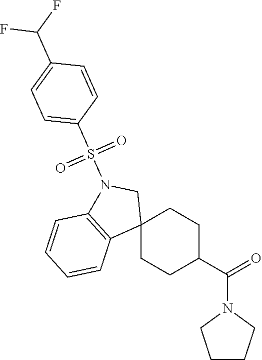 [SMILES: O=C(C1CCC2(CC1)CN(S(=O)(=O)c1ccc(C(F)F)cc1)c1ccccc12)N1CCCC1] | Yield: 58 mg, 38%; Appearance: Yellow solid; ¹H NMR (400 MHz, DMSO-d₆) δ 8.01-7.88 (m, 2H), 7.75-7.66 (m, 2H), 7.53 (d, J = 8.1 Hz, 1H), 7.34-6.80 (m, 4H), 3.82-3.62 (m, 3H), 3.46 (q, J = 6.5 Hz, 2H), 3.32 (t, J = 6.8 Hz, 2H), 3.20 (d, J = 5.3 Hz, 2H), 2.43-2.26 (m, 1H), 2.00-1.89 (m, 2H), 1.89-1.73 (m, 3H), 1.65-1.42 (m, 5H), 1.25 (d, J = 10.7 Hz, 2H).; HPLC purity: 100%; LCMS Calculated for C₂₅H₂₈F₂N₂O₃S: 474.57; Observed: 475.0[M + H]⁺. |
| I-238 | 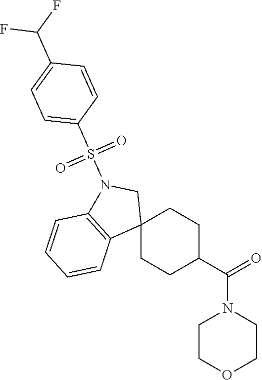 [SMILES: O=C(C1CCC2(CC1)CN(S(=O)(=O)c1ccc(C(F)F)cc1)c1ccccc12)N1CCOCC1] | Yield: 42.3 mg, 46.1%; Appearance: Yellow solid; ¹H NMR (600 MHz, DMSO-d₆) δ 8.02- 7.93 (m, 2H), 7.79-7.71 (m, 2H), 7.53-7.44 (m, 1H), 7.26-6.94 (m, 4H), 3.83-3.66 (m, 2H), 3.58-3.39 (m, 8H), 2.79-2.58 (m, 1H), 1.76- 1.36 (m, 6H), 1.28-1.06 (m, 2H); HPLC purity: 100%; LCMS Calculated for C₂₅H₂₈F₂N₂O₄S: 490.57; Observed: 491.0[M + H]⁺. |
| I-239 | 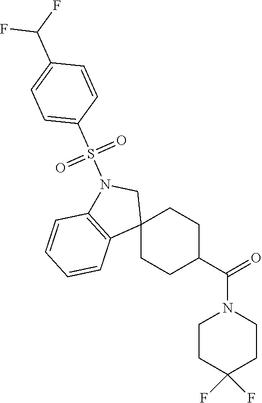 [SMILES: O=C(C1CCC2(CC1)CN(S(=O)(=O)c1ccc(C(F)F)cc1)c1ccccc12)N1CCC(F)(F)CC1] | Yield: 14.8 mg, 15%; Appearance: Yellow solid; ¹H NMR (600 MHz, DMSO-d₆) δ 7.98 (dd, J = 8.0, 5.8 Hz, 2H), 7.79-7.71 (m, 2H), 7.49 (dd, J = 8.2, 3.2 Hz, 1H), 7.26-6.97 (m, 4H), 3.83-3.65 (m, 2H), 3.64-3.50 (m, 4H), 2.86- 2.63 (m, 1H), 1.93 (d, J = 54.4 Hz, 4H), 1.72- 1.36 (m, 6H), 1.31-1.03 (m, 2H).; HPLC purity: 100%; LCMS Calculated for C₂₆H₂₈F₄N₂O₃S: 524.57; Observed: 525.2[M + H]⁺. |
| I-240 | 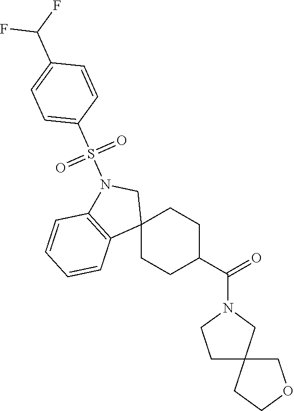 [SMILES: O=C(C1CCC2(CC1)CN(S(=O)(=O)c1ccc(C(F)F)cc1)c1ccccc12)N1CCC2(CCOC2)C1] | Yield: 47.3 mg, 49.2%; Appearance: Beige solid; ¹H NMR (400 MHz, DMSO-d₆) δ 8.00- 7.89 (m, 2H), 7.71 (d, J = 8.1 Hz, 2H), 7.53 (d, J = 8.2 Hz, 1H), 7.35-7.14 (m, 1H), 7.10-6.75 (m, 3H), 3.84-3.74 (m, 4H), 3.68-3.22 (m, 7H), 2.38 (sm, 1H), 2.01-1.71 (m, 5H), 1.67-1.44 (m, 5H), 1.25 (d, J = 9.8 Hz, 2H).; HPLC purity: 100%; LCMS Calculated for C₂₈H₃₂F₂N₂O₄S: 530.63; Observed: 531.2[M + H]⁺. |
| I-241 | 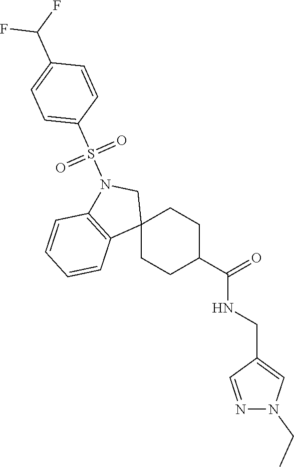 [SMILES: CCn1cc(CNC(=O)C2CCC3(CC2)CN(S(=O)(=O)c2ccc(C(F)F)cc2)c2ccccc23)cn1] | Yield: 49.7 mg, 47.2%; Appearance: Yellow oil; ¹H NMR (400 MHz, DMSO-d₆) δ 7.98-7.88 (m, 2H), 7.84 (s, 1H), 7.71 (d, J = 8.1 Hz, 2H), 7.53 (d, J = 8.1 Hz, 1H), 7.44 (d, J = 5.1 Hz, 1H), 7.26- 6.95 (m, 4H), 4.15-4.05 (m, 4H), 3.83-3.65 (m, 3H), 3.20 (dd, J = 5.3, 1.3 Hz, 2H), 2.36- 2.06 (m, 1H), 1.76-1.36 (m, 8H), 1.29-1.15 (m, 2H).; HPLC purity: 100%; LCMS Calculated for C₂₇H₃₀F₂N₄O₃S: 528.62; Observed: 529.2[M + H]⁺. |
| I-242 | 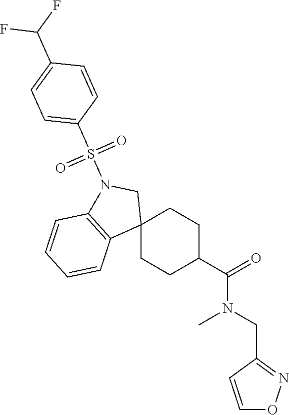 [SMILES: CN(Cc1ccon1)C(=O)C1CCC2(CC1)CN(S(=O)(=O)c1ccc(C(F)F)cc1)c1ccccc12] | Yield: 50 mg, 45%; Appearance: Yellow solid; ¹H NMR (400 MHz, DMSO-d₆) δ 8.77-8.54 (m, 1H), 7.99-7.87 (m, 2H), 7.71 (d, J = 8.1 Hz, 2H), 7.53 (d, J = 8.1 Hz, 1H), 7.36-6.76 (m, 4H), 6.52- 6.26 (m, 1H), 4.69-4.50 (m, 2H), 3.87-3.60 (m, 2H), 3.04 (d, J = 5.4 Hz, 2H), 2.88 (s, 1H), 2.82-2.59 (m, 1H), 1.88-1.44 (m, 6H), 1.39- 1.15 (m, 2H).; HPLC purity: 100%; LCMS Calculated for C₂₆H₂₇F₂N₃O₄S: 515.58; Observed: 516.0[M + H]⁺. |
| I-243 | 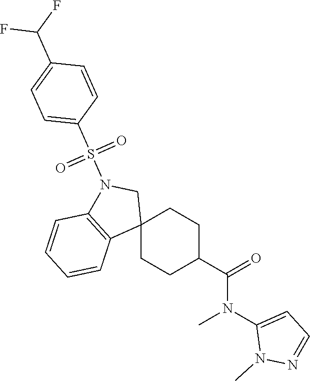 [SMILES: CN(C(=O)C1CCC2(CC1)CN(S(=O)(=O)c1ccc(C(F)F)cc1)c1ccccc12)c1ccnn1C] | Yield: 15.6 mg, 13.9%; Appearance: Yellow solid; ¹H NMR (600 MHz, DMSO-d₆) δ 7.95 (dd, J = 31.2, 8.1 Hz, 2H), 7.74 (d, J = 8.1 Hz, 2H), 7.52-7.42 (m, 2H), 7.30-6.91 (m, 4H), 6.29 (s, 1H), 3.78 (s, 2H), 3.62 (d, J = 6.1 Hz, 3H), 3.05 (d, J = 7.3 Hz, 3H), 2.20-1.95 (m, 1H), 1.69- 1.20 (m, 6H), 1.07-0.97 (m, 2H).; HPLC purity: 100%; LCMS Calculated for C₂₆H₂₈F₂N₄O₃S: 514.59; Observed: 515.0[M + H]⁺. |
| I-244 | 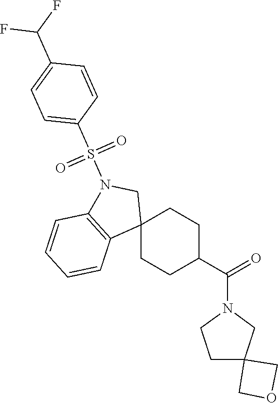 [SMILES: O=C(C1CCC2(CC1)CN(S(=O)(=O)c1ccc(C(F)F)cc1)c1ccccc12)N1CCC2(COC2)C1] | Yield: 6.7 mg, 6.65%; Appearance: Beige solid; ¹H NMR (600 MHz, DMSO-d₆) δ 8.03-7.92 (m, 2H), 7.79-7.70 (m, 2H), 7.53-7.43 (m, 1H), 7.29-7.13 (m, 2H), 7.11-6.91 (m, 2H), 4.53- 4.46 (m, 2H), 4.46-4.38 (m, 2H), 3.79 (d, J = 3.3 Hz, 1H), 3.70 (dd, J = 11.0, 6.3 Hz, 2H), 3.49 (s, 1H), 3.45 (q, J = 7.2 Hz, 1H), 3.26 (t, J = 7.1 Hz, 1H), 2.19-2.10 (m, 1H), 2.04 (q, J = 6.6 Hz, 1H), 1.71-1.61 (m, 2H), 1.59-1.48 (m, 3H), 1.44- 1.34 (m, 1H), 1.28-1.21 (m, 1H), 1.16-1.05 (m, 1H).; HPLC purity: 100%; LCMS Calculated for C₂₇H₃₀F₂N₂O₄S: 516.6; Observed: 517.2[M + H]⁺. |
| I-245 | 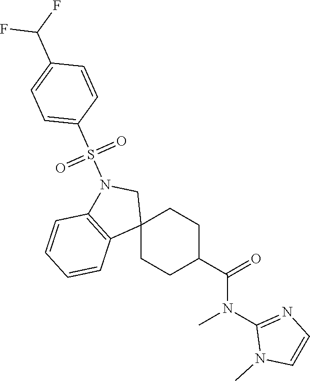 [SMILES: CN(C(=O)C1CCC2(CC1)CN(S(=O)(=O)c1ccc(C(F)F)cc1)c1ccccc12)c1nccn1C] | Yield: 29.5 mg, 27.7%; Appearance: Yellow solid; ¹H NMR (400 MHz, DMSO-d₆) δ 8.06- 7.87 (m, 2H), 7.75 (d, J = 8.1 Hz, 2H), 7.53-7.38 (m, 1H), 7.22-6.81 (m, 5H), 3.83-3.62 (m, 2H), 3.49 (s, 3H), 3.03 (d, J = 3.9 Hz, 3H), 2.04-0.91 (m, 9H).; HPLC purity: 100%; LCMS Calculated for C₂₆H₂₈F₂N₄O₃S: 514.59; Observed: 515.0[M + H]⁺. |
| I-246 | 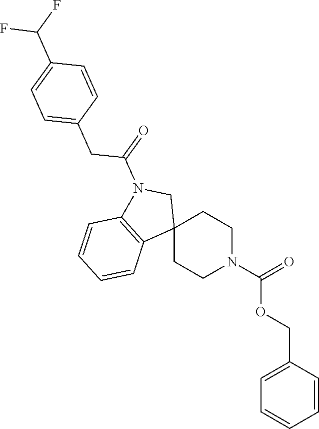 [SMILES: O=C(OCc1ccccc1)N1CCC2(CC1)CN(C(=O)Cc1ccc(C(F)F)cc1)c1ccccc12] | Yield: 33.2 mg, 21.1%; Appearance: Beige solid; ¹H NMR (600 MHz, DMSO-d₆) δ 8.02 (d, J = 8.1 Hz, 1H), 7.52 (d, J = 7.8 Hz, 2H), 7.43 (d, J = 7.8 Hz, 2H), 7.37 (d, J = 4.4 Hz, 4H), 7.34- 7.30 (m, 1H), 7.27 (d, J = 7.5 Hz, 1H), 7.18-7.13 (m, 1H), 7.11-6.88 (m, 2H), 5.10 (s, 2H), 4.13 (s, 2H), 4.02 (d, J = 13.4 Hz, 2H), 3.97 (s, 2H), 3.13-2.89 (m, 2H), 1.81-1.70 (m, 2H), 1.59 (d, J = 13.2 Hz, 2H).; HPLC purity: 100%; LCMS Calculated for C₂₉H₂₈F₂N₂O₃: 490.55; Observed: 491.0[M + H]⁺. |
| I-247 | 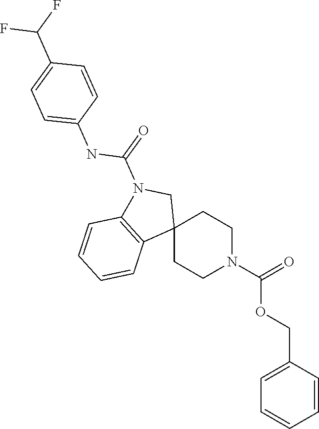 [SMILES: O=C(OCc1ccccc1)N1CCC2(CC1)CN(C(=O)Nc1ccc(C(F)F)cc1)c1ccccc12] | Yield: 30.3 mg, 19.2%; Appearance: Yellow solid; ¹H NMR (600 MHz, DMSO-d₆) δ 8.02 (d, J = 8.1 Hz, 1H), 7.52 (d, J = 7.8 Hz, 2H), 7.43 (d, J = 7.8 Hz, 2H), 7.37 (d, J = 4.4 Hz, 4H), 7.34- 7.30 (m, 1H), 7.27 (d, J = 7.5 Hz, 1H), 7.18-7.13 (m, 1H), 7.11-6.88 (m, 2H), 5.10 (s, 2H), 4.13 (s, 2H), 4.02 (d, J = 13.4 Hz, 2H), 3.97 (s, 2H), 3.13-2.89 (m, 2H), 1.81-1.70 (m, 2H), 1.59 (d, J = 13.2 Hz, 2H).; HPLC purity: 100%; LCMS Calculated for C₂₈H₂₇F₂N₃O₃: 491.54; Observed: 492.2[M + H]⁺. |
| I-248 | 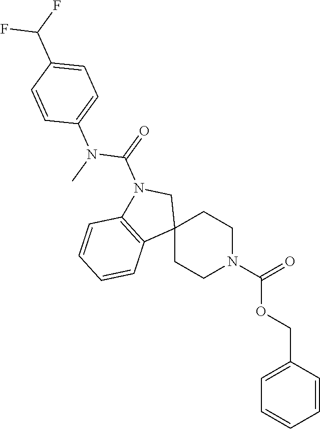 [SMILES: CN(C(=O)N1CC2(CCN(C(=O)OCc3ccccc3)CC2)c2ccccc21)c1ccc(C(F)F)cc1] | Yield: 159.3 mg, 92%; Appearance: Yellow oil; ¹H NMR (400 MHz, DMSO-d6) δ 7.54 (d, J = 8.2 Hz, 2H), 7.47-7.27 (m, 8H), 7.16 (d, J = 7.6 Hz, 1H), 7.14-6.84 (m, 3H), 5.07 (s, 2H), 3.89 (d, J = 13.5 Hz, 2H), 3.49 (s, 2H), 3.26 (s, 3H), 2.73-2.57 (m, 2H), 2.09-2.02 (m, 2H), 1.69- 1.52 (m, 2H), 1.40 (d, J = 13.1 Hz, 2H); HPLC purity: 100%; LCMS Calculated for C₂₉H₂₉F₂N₃O₃: 505.57; Observed: 507.2[M + H]⁺. |
| I-249 | 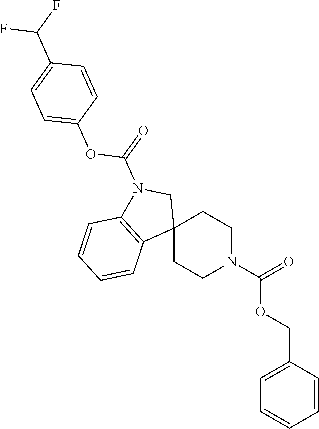 [SMILES: O=C(OCc1ccccc1)N1CCC2(CC1)CN(C(=O)Oc1ccc(C(F)F)cc1)c1ccccc12] | Yield: mg, %; Appearance: Yellow solid; ¹H NMR (400 MHz, DMSO-d₆) δ 7.75-7.69 (m, 1H), 7.66 (d, J = 8.1 Hz, 2H), 7.43 (d, J = 8.1 Hz, 2H), 7.39 (d, J = 4.4 Hz, 4H), 7.37-7.31 (m, 2H), 7.24 (dd, J = 13.8, 6.2 Hz, 1H), 7.10-6.91 (m, 2H), 5.11 (s, 2H), 4.19 (s, 2H), 4.05 (d, J = 13.8 Hz, 2H), 3.18-2.96 (m, 2H), 1.85-1.64 (m, 4H).; HPLC purity: 100%; LCMS Calculated for C₂₈H₂₆F₂N₂O₄: 492.52; Observed: 493.0[M + H]⁺. |
| I-250 | 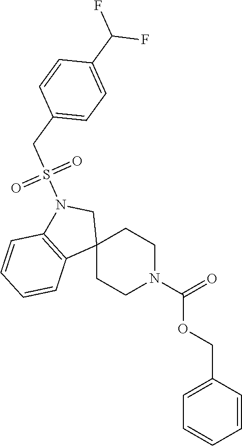 [SMILES: O=C(OCc1ccccc1)N1CCC2(CC1)CN(S(=O)(=O)Cc1ccc(C(F)F)cc1)c1ccccc12] | Yield: 24.3 mg, 15.4%; Appearance: Light brown solid; ¹H NMR (600 MHz, DMSO-d₆) δ 7.52 (d, J = 7.9 Hz, 2H), 7.46 (d, J = 7.9 Hz, 2H), 7.39-7.35 (m, 4H), 7.34-7.28 (m, 1H), 7.26 (d, J = 7.5 Hz, 1H), 7.12 (d, J = 4.1 Hz, 2H), 7.11- 6.91 (m, 2H), 5.08 (s, 2H), 4.71 (s, 2H), 3.97 (d, J = 13.4 Hz, 2H), 3.80 (s, 2H), 3.06-2.78 (m, 2H), 1.76-1.65 (m, 2H), 1.54 (d, J = 13.1 Hz, 2H).; HPLC purity: 100%; LCMS Calculated for C₂₈H₂₈F₂N₂O₄S: 526.6; Observed: 527.2[M + H]⁺. |
| I-251 | 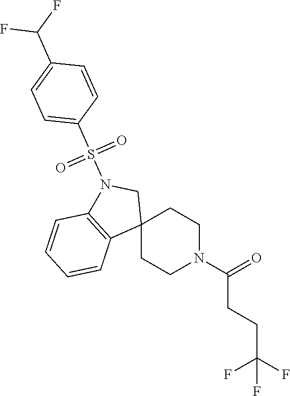 [SMILES: O=C(CCC(F)(F)F)N1CCC2(CC1)CN(S(=O)(=O)c1ccc(C(F)F)cc1)c1ccccc12] | Yield: 47.9 mg, 45.5%; Appearance: White solid; ¹H NMR (400 MHz, DMSO-d₆) δ 7.96 (d, J = 8.1 Hz, 2H), 7.73 (d, J = 8.1 Hz, 2H), 7.53 (d, J = 8.1 Hz, 1H), 7.21 (t, J = 7.6 Hz, 1H), 7.12- 7.08 (m, 1H), 7.04-6.79 (m, 2H), 4.35 (d, J = 13.7 Hz, 1H), 3.98-3.84 (m, 2H), 3.77 (d, J = 14.1 Hz, 1H), 3.17 (t, J = 13.2 Hz, 1H), 2.69 (t, J = 13.1 Hz, 1H), 2.61-2.55 (m, 2H), 2.48-2.36 (m, 2H), 1.70 (t, J = 13.0 Hz, 1H), 1.62-1.46 (m, 1H), 1.24 (dd, J = 41.4, 13.5 Hz, 2H).; HPLC purity: 100%; LCMS Calculated for C₂₃H₂₃F₅N₂O₃S: 502.5; Observed: 503.0[M + H]⁺. |
| I-252 | 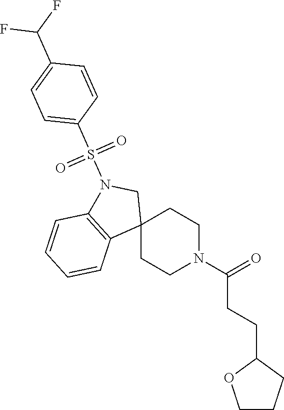 [SMILES: O=C(CCC1CCCO1)N1CCC2(CC1)CN(S(=O)(=O)c1ccc(C(F)F)cc1)c1ccccc12] | Yield: 52.6 mg, 50.2%; Appearance: White solid; ¹H NMR (400 MHz, DMSO-d₆) δ 7.96 (d, J = 8.0 Hz, 2H), 7.73 (d, J = 8.1 Hz, 2H), 7.53 (d, J = 8.1 Hz, 1H), 7.24-7.17 (m, 1H), 7.13-6.78 (m, 3H), 4.35 (d, J = 13.6 Hz, 1H), 3.98-3.85 (m, 2H), 3.84-3.66 (m, 3H), 3.60 (q, J = 7.4 Hz, 1H), 3.23-3.08 (m, 1H), 2.63 (t, J = 13.0 Hz, 1H), 2.43-2.25 (m, 2H), 2.02-1.89 (m, 1H), 1.85 (q, J = 6.6 Hz, 2H), 1.78-1.36 (m, 5H), 1.29 (d, J = 13.7 Hz, 1H), 1.18 (d, J = 13.4 Hz, 1H).; HPLC purity: 100%; LCMS Calculated for C₂₆H₃₀F₂N₂O₄S: 504.59; Observed: 505.2[M + H]⁺. |
| I-253 | 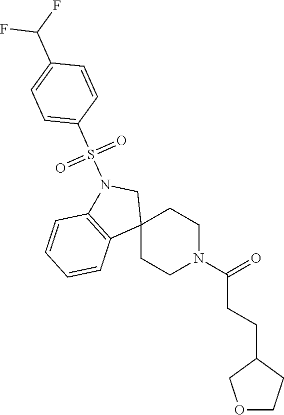 [SMILES: O=C(CCC1CCOC1)N1CCC2(CC1)CN(S(=O)(=O)c1ccc(C(F)F)cc1)c1ccccc12] | Yield: 52.5 mg, 50.1%; Appearance: White solid; ¹H NMR (400 MHz, DMSO-d₆) δ 7.96 (d, J = 8.0 Hz, 2H), 7.73 (d, J = 8.0 Hz, 2H), 7.53 (d, J = 8.1 Hz, 1H), 7.21 (t, J = 7.9 Hz, 1H), 7.13- 6.78 (m, 3H), 4.34 (d, J = 13.7 Hz, 1H), 4.00- 3.84 (m, 2H), 3.82-3.68 (m, 3H), 3.63 (q, J = 7.6 Hz, 1H), 3.25 (t, J = 7.6 Hz, 1H), 3.15 (t, J = 13.6 Hz, 1H), 2.63 (t, J = 13.3 Hz, 1H), 2.30 (t, J = 7.9 Hz, 2H), 2.22-2.08 (m, 1H), 2.08-1.95 (m, 1H), 1.69-1.42 (m, 5H), 1.29 (d, J = 13.6 Hz, 1H), 1.18 (d, J = 13.6 Hz, 1H).; HPLC purity: 99.17%; LCMS Calculated for C₂₆H₃₀F₂N₂O₄S: 504.59; Observed: 505.2[M + H]⁺. |
| I-254 | 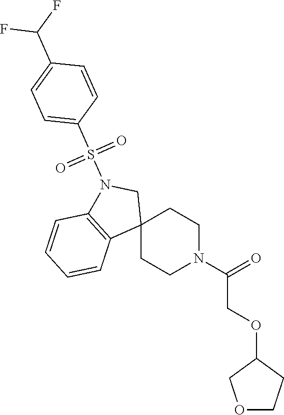 [SMILES: O=C(COC1CCOC1)N1CCC2(CC1)CN(S(=O)(=O)c1ccc(C(F)F)cc1)c1ccccc12] | Yield: 52.6 mg, 48.9%; Appearance: White solid; ¹H NMR (400 MHz, DMSO-d₆) δ 7.96 (d, J = 8.0 Hz, 2H), 7.73 (d, J = 8.1 Hz, 2H), 7.53 (d, J = 8.1 Hz, 1H), 7.21 (t, J = 7.7 Hz, 1H), 7.13- 6.78 (m, 3H), 4.28 (d, J = 13.5 Hz, 1H), 4.22 4.00 (m, 3H), 3.91 (s, 2H), 3.81-3.63 (m, 5H), 3.13 (t, J = 13.0 Hz, 1H), 2.69 (t, J = 13.0 Hz, 1H), 2.01-1.89 (m, 2H), 1.75-1.63 (m, 1H), 1.62- 1.48 (m, 1H), 1.22 (dd, J = 23.6, 13.1 Hz, 2H).; HPLC purity: 100%; LCMS Calculated for C₂₅H₂₈F₂N₂O₅S: 506.56; Observed: 507.1[M + H]⁺. |
| I-255 | 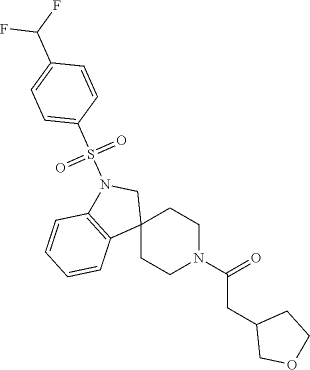 [SMILES: O=C(CC1CCOC1)N1CCC2(CC1)CN(S(=O)(=O)c1ccc(C(F)F)cc1)c1ccccc12] | Yield: 61.9 mg, 59%; Appearance: White solid; ¹H NMR (400 MHz, DMSO-d₆) δ 7.96 (d, J = 8.0 Hz, 2H), 7.73 (d, J = 8.0 Hz, 2H), 7.53 (d, J = 8.0 Hz, 1H), 7.26-7.16 (m, 1H), 7.15-6.79 (m, 3H), 4.34 (d, J = 13.6 Hz, 1H), 3.99-3.86 (m, 2H), 3.86-3.69 (m, 3H), 3.63 (q, J = 7.7 Hz, 1H), 3.32-3.21 (m, 1H), 3.15 (t, J = 13.8 Hz, 1H), 2.73-2.57 (m, 1H), 2.46-2.28 (m, 2H), 2.12- 1.97 (m, 1H), 1.70-1.59 (m, 1H), 1.59-1.44 (m, 2H), 1.29 (d, J = 13.5 Hz, IH), 1.18 (d, J = 13.5 Hz, 1H).; HPLC purity: 100%; LCMS Calculated for C₂₅H₂₈F₂N₂O₄S: 490.57; Observed: 491.2[M + H]⁺. |
| I-256 | 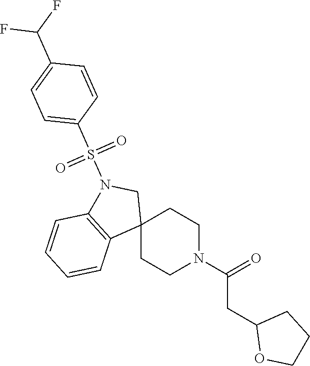 [SMILES: O=C(CC1CCCO1)N1CCC2(CC1)CN(S(=O)(=O)c1ccc(C(F)F)cc1)c1ccccc12] | Yield: 52.8 mg, 50.3%; Appearance: White solid; ¹H NMR (400 MHz, DMSO-d₆) δ 7.96 (d, J = 8.1 Hz, 2H), 7.73 (d, J = 8.1 Hz, 2H), 7.53 (d, J = 7.9 Hz, 1H), 7.27-7.18 (m, 1H), 7.12-6.77 (m, 3H), 4.35 (d, J = 13.6 Hz, 1H), 4.14-4.04 (m, 1H), 3.97-3.86 (m, 2H), 3.86-3.71 (m, 2H), 3.67-3.58 (m, 1H), 3.15 (t, J = 13.2 Hz, 1H), 2.70-2.57 (m, 2H), 2.41-2.29 (m, 1H), 2.12- 2.01 (m, 1H), 1.93-1.80 (m, 2H), 1.73-1.60 (m, 1H), 1.60-1.42 (m, 2H), 1.28 (t, J = 12.7 Hz, 1H), 1.18 (d, J = 13.6 Hz, 1H).; HPLC purity: 100%; LCMS Calculated for C₂₅H₂₈F₂N₂O₄S: 490.57; Observed: 491.2[M + H]⁺. |
| I-257 | 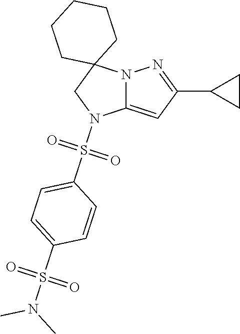 [SMILES: CN(C)S(=O)(=O)c1ccc(S(=O)(=O)N2CC3(CCCCC3)n3nc(C4CC4)cc32)cc1] | Yield: 160.5 mg, 17.7%; Appearance: Yellow solid; ¹H NMR (400 MHz, DMSO-d₆) δ 8.11 (d, J = 8.2 Hz, 2H), 7.98 (d, J = 8.2 Hz, 2H), 5.67 (s, 1H), 4.11 (s, 2H), 2.61 (s, 6H), 1.80 (tt, J = 8.5, 5.0 Hz, 1H), 1.61 (d, J = 11.9 Hz, 2H), 1.57-1.48 (m, 2H), 1.46 (d, J = 9.2 Hz, 1H), 1.36-1.16 (m, 3H), 1.12 (d, J = 13.1 Hz, 2H), 0.83 (dt, J = 8.4, 3.2 Hz, 2H), 0.65 (dt, J = 5.0, 3.0 Hz, 2H); HPLC purity: 100%; LCMS Calculated for C₂₁H₂₈N₄O₄S₂: 464.6; Observed: 465.2 [M + H]⁺. |

### Example 62—Biological Activity

### Example 62a

For the TFEB nuclear translocation assay, HeLa wt or HeLa TRPML1 KO cells were plated at 2700 cells/well into black-walled, 384-well Cell carrier Ultra tissue culture treated plates in complete media and incubated overnight. The next day, cells are treated for 2 hrs with compounds and incubated at 37° C. Cells were then fixed for 30 minutes at room temperature in 4% final PFA and washed five times with 90 μL PBS. PBS is aspirated from the wells and the cells are blocked with 7.5 μL blocking buffer (1.1 PBS/Odyssey block buffer containing 0.1% triton x-100 and 1% goat serum). After 30-60 minutes of block, 7.5 μL of primary anti-TFEB (rabbit) antibody is added for a final dilution of 1:200 antibody in 15 μL blocking buffer. Plates are incubated overnight at 4° C. The following day, plates are washed again into PBS, 90 μL with 5 washes, all PBS is aspirated from the wells and the cells are incubated for 1 hr in 1:1000 goat-anti rabbit Alexa 488 secondary antibody, also containing 10 μg/mL Hoechst 33342. After the 1 hr RT incubation, plates are washed a final time into PBS, sealed with foil and imaged with an automated epifluorescence microscopy (PerkinElmer Operetta CLS). Four different fields were imaged per well using ×20 magnification for DAPI and FITC filter sets. Images were quantified using PerkinElmer Harmony software, briefly: apply flatfield correction (basic/advanced) for input images. Use the Find Nuclei building block with channel set at Hoechst to find the nuclei. Use the Find cytoplasm building block with channel set to Alexa 488 to find the cytoplasm. Use select cell region with Channel set at Alexa 488 and region of interest as Nuclei and define outer border at 0 μm and inner at 45 μm to cover complete nuclei. Use select cell region with Channel set at Alexa 488 and region of interest as ring region and define outer border at −5 μm and inner at 0 μm to define a ring around the nucleus. Use the find calculate intensity parameter to calculate intensity of the nuclear region and the ring region. Define results as Number of nuclei and ratio of A/B where A is Intensity of Nuclei and B is intensity of the ring region.

Table 3 shows the activity of selected compounds of this invention in TFEB assays. Compounds having an activity designated as “++++” provided an EC₅₀ of ≤2.00 μM; compounds having an activity designated as “+++” provided an EC₅₀ of 2.01-8.00 μM; compounds having an activity designated as “++” provided an EC₅₀ of 8.01-9.99 μM; and compounds having an activity designated as “+” provided an EC₅₀ of ≥10.00 μM.

TABLE 3

| Compound No. | TFEB EC₅₀ (μM) |
|---|---|
| I-1 | ++ |
| I-2 | +++ |
| I-3 | +++ |
| I-4 | ++ |
| I-5 | ++++ |
| I-6 | + |
| I-7 | + |
| I-8 | + |
| I-9 | +++ |
| I-10 | +++ |
| I-11 | + |
| I-12 | + |
| I-13 | + |
| I-14 | +++ |
| I-15 | ++ |
| I-16 | + |
| I-17 | + |
| I-18 | +++ |
| I-19 | + |
| I-20 | + |
| I-21 | + |
| I-22 | + |
| I-23 | + |
| I-24 | + |
| I-25 | +++ |
| I-26 | +++ |
| I-27 | +++ |
| I-28 | + |
| I-29 | +++ |
| I-30 | + |
| I-31 | +++ |
| I-32 | + |
| I-33 | + |
| I-34 | +++ |
| I-35 | +++ |
| I-36 | + |
| I-37 | +++ |
| I-38 | + |
| I-39 | + |
| I-40 | +++ |
| I-41 | ++ |
| I-42 | +++ |
| I-43 | + |
| I-44 | +++ |
| I-45 | +++ |
| I-46 | +++ |
| I-47 | +++ |
| I-48 | +++ |
| I-49 | +++ |
| I-50 | + |
| I-51 | ++ |
| I-52 | + |
| I-53 | + |
| I-54 | + |
| I-55 | + |
| I-56 | + |
| I-57 | +++ |
| I-58 | +++ |
| I-59 | + |
| I-60 | ++ |
| I-61 | + |
| I-62 | + |
| I-63 | + |
| I-64 | +++ |
| I-65 | ++++ |
| I-66 | +++ |
| I-67 | +++ |
| I-68 | ++++ |
| I-69 | ++++ |
| I-70 | + |
| I-71 | + |
| I-72 | +++ |
| I-73 | +++ |
| I-74 | +++ |
| I-75 | ++ |
| I-76 | +++ |
| I-77 | ++++ |
| I-78 | +++ |
| I-79 | ++++ |
| I-80 | ++++ |
| I-81 | ++++ |
| I-82 | + |
| I-83 | + |
| I-84 | + |
| I-85 | + |
| I-86 | ++++ |
| I-87 | ++++ |
| I-88 | + |
| I-89 | + |
| I-90 | + |
| I-91 | +++ |
| I-92 | +++ |
| I-93 | ++++ |
| I-94 | ++++ |
| I-95 | ++++ |
| I-96 | +++ |
| I-97 | ++++ |
| I-98 | ++++ |
| I-99 | ++++ |
| I-100 | ++++ |
| I-101 | +++ |
| I-102 | +++ |
| I-103 | +++ |
| I-104 | ++++ |
| I-105 | ++++ |
| I-106 | ++++ |
| I-115 | +++ |
| I-116 | ++++ |
| I-117 | + |
| I-118 | ++++ |
| I-119 | ++++ |
| I-120 | +++ |
| I-121 | ++++ |
| I-122 | ++++ |
| I-123 | +++ |
| I-124 | + |
| I-125 | + |
| I-126 | ++++ |
| I-127 | +++ |
| I-128 | ++++ |
| I-129 | ++++ |
| I-130 | ++++ |
| I-131 | ++++ |
| I-132 | ++++ |
| I-133 | ++++ |
| I-134 | ++++ |
| I-137 | ++++ |
| I-139 | ++++ |
| I-144 | + |
| I-145 | + |
| I-146 | + |
| I-147 | + |
| I-148 | + |
| I-149 | ++++ |
| I-150 | + |
| I-151 | + |
| I-153 | ++++ |
| I-154 | + |
| I-157 | ++++ |
| I-158 | ++++ |
| I-160 | ++++ |
| I-161 | +++ |
| I-162 | + |
| I-163 | ++++ |
| I-164 | ++++ |
| I-165 | +++ |
| I-166 | ++++ |
| I-167 | ++ |
| I-168 | ++++ |
| I-169 | ++++ |
| I-170 | ++++ |
| I-174 | ++++ |
| I-178 | +++ |
| I-180 | ++++ |
| I-181 | ++++ |
| I-182 | ++++ |
| I-184 | + |
| I-186 | ++++ |
| I-187 | ++++ |
| I-188 | ++++ |
| I-189 | ++++ |
| I-190 | + |
| I-191 | +++ |
| I-219 | + |
| I-220 | +++ |
| I-221 | +++ |
| I-222 | ++++ |
| I-223 | ++++ |
| I-225 | + |
| I-226 | + |
| I-227 | +++ |
| I-228 | ++++ |
| I-230 | ++++ |
| I-234 | ++++ |
| I-235 | ++++ |
| I-246 | + |
| I-247 | + |
| I-248 | + |
| I-249 | + |
| I-250 | ++++ |
| I-257 | ++++ |

### Example 62b

For the Lamp1 lysosomal exocytosis assay, a human monocytic suspension cell line (THP-1) was used. Cells in culture should be used between a density of 800e5/mL and 1.2e6/mL on the day of the assay. For each 96-well assay plate to be used, pellet 11e6 THP-1 cells at 330×g for 5 min in a 50 mL conical tube, aspirate media and resuspend in 22 mLs of RPMI 1640/10% FBS media for a final cell concentration of 5e5/mL. Prior to addition of cells to the assay plate, perform a 7 pt dilution curve using a top dose of compound at 2 mM and use a 3-fold serial dilution, the 8^{th} point is DMSO only. Final top dose of compound in the assay was 20 μM. 2 μL of each serial dilution was transferred to a new 96-well v-bottom assay plate. 198 μL of cells (1e5 total cells/well) was prepared as above in each well of the assay plate and incubated at 37° C./5% CO₂/95% RH for 55 min. After the 55 min incubation, cells were pelleted for 5 min at 4 C at 400×g in a tabletop centrifuge. The media was separated from the cell pellets in the plate. The pellets were washed using 200 μL ice cold buffer (PBS Ca⁺⁺/Mg⁺⁺ containing 2% final BSA and 80 μM final Dynasore). Cells were pelleted again at 400×g-5 min in a 4° C. tabletop centrifuge. Buffer was separated from cells. Each well was suspended in 75 uL Lamp1-PE antibody buffer (PBS Ca⁺⁺/Mg⁺⁺ containing 2% final BSA with 80 uM final Dynasore and 1:800 final dilution of Lamp 1-PE labeled antibody). After a 45 min antibody incubation on ice, 25 μL of sytox green was added (I drop/mL buffer), and each well was mixed and incubated for an additional 15 min on ice. After the 15 min sytox green incubation, 100 μL of ice-cold buffer (PBS Ca⁺⁺/Mg⁺⁺ containing 2% final BSA and 80 uM final Dynasore) was added and cells were pelleted for 5 min at 400×g. The buffer was removed and each sample was resuspended in 100 uL of ice-cold buffer (PBS Ca⁺⁺/Mg⁺⁺ containing 2% final BSA and 80 μM final Dynasore). FACs gating strategy: FACS analyze for sytox green (A488) and Lamp1-PE (DsRed). First gate against FSC-A/SSC-A to find non-fragmented or ‘live’ THP-1 cells. Next gate against FITC-A/FSC-A to omit sytox green positive cells. Then gate for FSC-A/FSC-H to remove any doublet cells. The last gate was a histogram for PE. The statistics for ‘count’ and ‘geometric mean fluorescence intensity’ (gWFI) for PE were repoted on the last gate.

Table 4 shows the activity of selected compounds of this invention in exocytosis assays. Compounds having an activity designated as “++++” provided an exocytosis readout of ≤0.50 μM; compounds having an activity designated as “+++” provided an exocytosis readout of 0.51-1.00 μM; compounds having an activity designated as “++” provided an exocytosis readout of 1.01-5.00 μM; and compounds having an activity designated as “+” provided an exocytosis readout of ≥5.01 μM

TABLE 4

| Compound No. | Exocytosis (μM) |
|---|---|
| I-2 | + |
| I-5 | + |
| I-31 | + |
| I-35 | + |
| I-48 | + |
| I-57 | + |
| I-67 | + |
| I-68 | ++++ |
| I-73 | + |
| I-74 | + |
| I-79 | ++++ |
| I-80 | + |
| I-81 | ++++ |
| I-83 | + |
| I-86 | + |
| I-87 | +++ |
| I-91 | + |
| I-92 | + |
| I-94 | + |
| I-95 | ++++ |
| I-97 | +++ |
| I-98 | ++ |
| I-99 | ++++ |
| I-100 | + |
| I-101 | + |
| I-103 | + |
| I-104 | ++++ |
| I-105 | + |
| I-107 | + |
| I-108 | + |
| I-109 | + |
| I-116 | + |
| I-118 | ++ |
| I-119 | ++ |
| I-122 | + |
| I-124 | + |
| I-126 | + |
| I-127 | ++ |
| I-129 | +++ |
| I-130 | ++++ |
| I-131 | + |
| I-132 | + |
| I-133 | + |
| I-134 | ++++ |
| I-135 | ++++ |
| I-136 | + |
| I-137 | ++++ |
| I-138 | + |
| I-139 | ++++ |
| I-141 | ++ |
| I-142 | + |
| I-143 | + |
| I-145 | + |
| I-148 | + |
| I-149 | ++++ |
| I-152 | + |
| I-154 | + |
| I-155 | ++++ |
| I-157 | + |
| I-158 | +++ |
| I-159 | + |
| I-160 | ++++ |
| I-161 | ++ |
| I-162 | + |
| I-163 | + |
| I-164 | + |
| I-165 | + |
| I-166 | + |
| I-167 | + |
| I-168 | + |
| I-169 | + |
| I-170 | +++ |
| I-171 | +++ |
| I-172 | + |
| I-173 | ++ |
| I-174 | ++++ |
| I-175 | + |
| I-176 | ++ |
| I-177 | +++ |
| I-178 | +++ |
| I-179 | + |
| I-180 | ++++ |
| I-181 | ++++ |
| I-182 | + |
| I-183 | ++++ |
| I-184 | + |
| I-185 | + |
| I-186 | ++++ |
| I-187 | +++ |
| I-188 | + |
| I-189 | ++++ |
| I-190 | + |
| I-191 | + |
| I-192 | + |
| I-193 | + |
| I-194 | + |
| I-195 | + |
| I-196 | + |
| I-197 | + |
| I-198 | + |
| I-199 | + |
| I-200 | + |
| I-201 | + |
| I-202 | + |
| I-203 | + |
| I-204 | + |
| I-205 | + |
| I-206 | + |
| I-207 | + |
| I-208 | + |
| I-209 | + |
| I-210 | + |
| I-211 | + |
| I-212 | + |
| I-213 | + |
| I-214 | + |
| I-215 | + |
| I-216 | + |
| I-217 | + |
| I-218 | + |
| I-219 | + |
| I-220 | + |
| I-221 | + |
| I-222 | + |
| I-223 | +++ |
| I-224 | + |
| I-225 | + |
| I-226 | + |
| I-227 | + |
| I-228 | + |
| I-229 | + |
| I-230 | + |
| I-231 | + |
| I-232 | + |
| I-233 | + |
| I-234 | + |
| I-235 | + |
| I-236 | + |
| I-237 | + |
| I-238 | + |
| I-239 | + |
| I-240 | + |
| I-241 | + |
| I-242 | + |
| I-243 | + |
| I-244 | + |
| I-245 | + |
| I-246 | + |
| I-247 | + |
| I-248 | + |
| I-249 | + |
| I-250 | ++++ |
| I-251 | ++ |
| I-252 | + |
| I-253 | + |
| I-254 | + |
| I-255 | + |
| I-256 | + |

1. A compound of formula I: 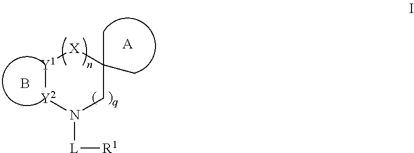
or a pharmaceutically acceptable salt thereof,
wherein
X is —NR⁵—, —C(R⁵)₂—, —C(O)—, or —O—;
each of Y¹ and Y² is independently selected from N and C;
L is an optionally substituted group selected from —C₀-C₆ alkylenyl-S(O)₂—, —S(O)₂—C₀-C₆ alkylenyl, —S(O)—C₀-C₆ alkylenyl, —C₀-C₆ alkylenyl-S(O)—, —C(O)—C₀-C₆ alkylenyl, —C(O)—O—C₀-C₆ alkylenyl, —C(O)—N(R⁸)—C₀-C₆ alkylenyl, —C₁-C₆ alkylenyl, and C₃-C₆ cycloalkylenyl;
A is C₃-C₁₂ cycloaliphatic or 3- to 12-membered heterocyclyl comprising 1 to 3 heteroatoms selected from N, O, and S, wherein A is substituted with (R²)ₘ;
B is a fused optionally substituted C₅-C₆ aryl or optionally substituted 5- to 6-membered heteroaryl comprising 1 to 3 heteroatoms selected from N, O, and S;
R¹ is C₅-C₁₂ aryl substituted with (R³)ₚ, 5- to 12-membered heteroaryl comprising 1 to 3 heteroatoms selected from N, O, and S substituted with (R³)ₚ, or 3- to 12-membered heterocyclyl comprising 1 to 3 heteroatoms selected from N, O, and S substituted with (R³)ₚ;
each R² is independently halo, oxo, —NR^{2a}R^{2b}, —C(O)O—R^{2a}, —O—C(O)Ra, —S(O)₂, —S(O)₂—R^{2a}, —C(O)—NR^{2a}R^{2b}, —N(R^{2a})—C(O)—R^{2b}, —C(O)—R^{2a}, —O—Ra, —O—C(O)—NR^{2a}R^{2b}, —NH—C(O)—NR^{2a}R^{2b}, —NH—C(O)—OR^{2a}, —NH—S(O)₂—R^{2a}, —C₁-C₆ alkylenyl-C(O)NR^{2a}R^{2b} or an optionally substituted group selected from C₁-C₆ aliphatic, C₅-C₁₂ aryl, and 3- to 12-membered heterocyclyl comprising 1 to 3 heteroatoms selected from N, O, and S;
each R^{2a} and each R^{2b} are independently selected from H and an optionally substituted group selected from C₁-C₆ aliphatic, C₃-C₁₂ cycloaliphatic, C₅-C₁₄ aryl, 5- to 12-membered heteroaryl comprising 1 to 4 heteroatoms selected from N, O, and S, and 3- to 12-membered heterocyclyl comprising 1 to 4 heteroatoms selected from N, O, and S;
each R³ is independently halo, —S(O)₂—NR^{3a}R^{3b}, —S(O)₂—R^{3b}, —S(NR^{3c})O)—NR^{3a}R^{3b}, —S(O)(NR^{3c})—R^{3b}, —S(O)—R^{3b}, —NR^{3a}S(O)₂—R^{3b}, —O—R^{3a}, —C(O)—Ra, —C(O)NH—R^{3a}, oxo, or an optionally substituted group selected from C₁-C₆ aliphatic, C₅-C₁₂ aryl, C₃-C₁₂ cycloaliphatic, 5- to 12-membered heteroaryl comprising 1 to 3 heteroatoms selected from N, O, and S, and 3- to 12-membered heterocyclyl comprising 1 to 3 heteroatoms selected from N, O, and S;
R^{3a} and R^{3b} are each independently selected from H and optionally substituted C₁-C₆ aliphatic, or R^{3a} and R^{3b} come together with the atoms to which they are attached to form optionally substituted C₃-C₁₂ cycloaliphatic or 3- to 12-membered heterocyclyl comprising 1 to 4 heteroatoms selected from N, O, and S;
each R^{3c} is independently selected from H, —OH, and optionally substituted C₁-C₆ aliphatic;
each R⁵ is independently selected from hydrogen, halo, —CN, and optionally substituted C₁-C₆ aliphatic;
R⁸ is selected from H and optionally substituted C₁-C₆ aliphatic;
n is 0 or 1;
m is 0 to 4;
p is 0 to 4; and
q is 1 or 2.

2. The compound of claim 1, wherein n is 0.

3. The compound of claim 1, wherein q is 1.

4. The compound of claim 1, wherein L is optionally substituted —S(O)₂—C₀-C₆ alkylenyl.

5-7. (canceled)

8. The compound of claim 1, wherein L is selected from —S(O)₂—, —S(O)₂—CH₂—, —S(O)₂—CH(CH₃)—, —CH(CH₃)—S(O)₂—, —CH₂—S(O)₂—,

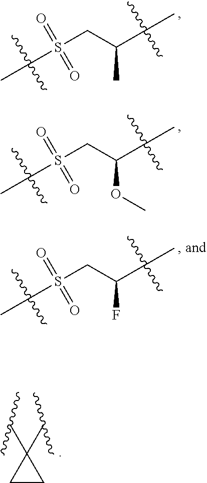

9. The compound of claim 1, wherein A is 3- to 12-membered heterocyclyl comprising 1 to 3 heteroatoms selected from N, O, and S.

10-11. (canceled)

12. The compound of claim 1, wherein A is C₃-C₁₂ cycloaliphatic.

13-16. (canceled)

17. The compound of claim 1, wherein B is a fused optionally substituted C₅-C₆ aryl.

18. The compound of claim 1, wherein B is a fused optionally substituted 5- to 12-membered heteroaryl comprising 1 to 3 heteroatoms selected from N, O, and S.

19-21. (canceled)

22. The compound of claim 1, wherein R¹ is phenyl substituted with (R³)ₚ.

23-28. (canceled)

29. The compound of claim 1, wherein m is 1 or 2.

30. The compound of claim 29, wherein each R² is halo, —C(O)O—R^{2a} or an optionally substituted group selected from C₁-C₆ aliphatic and C₅-C₁₂ aryl.

31-32. (canceled)

33. The compound of claim 1, wherein the compound is of formula II: 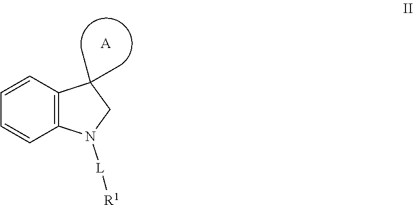
or a pharmaceutically acceptable salt thereof.

34. The compound of claim 1, wherein the compound is of formula IIa: 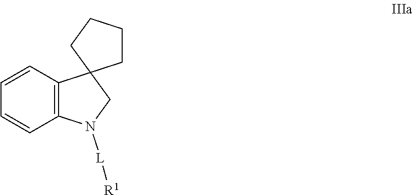
or a pharmaceutically acceptable salt thereof.

35. The compound of claim 1, wherein the compound is of formula IIb: 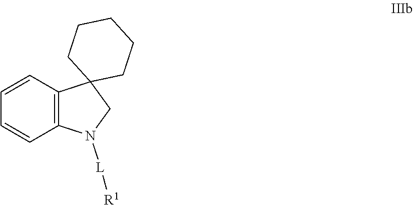
or a pharmaceutically acceptable salt thereof.

36-37. (canceled)

38. A compound selected from Table 1.

39. (canceled)

40. A pharmaceutical composition comprising a compound of claim 1, and a pharmaceutically acceptable carrier or excipient.

41. A method of modulating TRPML in a subject comprising administering to the subject a compound of claim 1, or a composition thereof.

42. A method of treating a disease, disorder, or condition in a subject comprising administering to the subject a compound of claim 1, or a composition thereof.

43. The method of claim 42, wherein the disease, disorder, or condition is associated with TPRML modulation.

44-54. (canceled)
